SEQUENCE LISTING (342 sequences)

SEQ ID NO: 1 - DNA, 366 bp, synthetic construct
  source 1..366: mol_type = other DNA
gaggtgcagctggtggagtctgggggaggcttggtccagcctggggggtccctgagactctcctgtgcagcctctggattcacctttagcagctatgccatgaactgggtccgccaggctccagggaaggggctggagtgggtctcaattattagtggtactattggtaagacatactacgcagactccgtgaagggccggttcaccatctctagagacaattccaagaacacgctctatctgcaaatgaacagcctgagaggcgaggacacggccgtttattactgtgcgaaagatacggaaagggatataactggatctccttttgactactggggccagggaaccctggtcaccgtctcctca

SEQ ID NO: 2 - protein, 122 aa, synthetic construct
EVQLVESGGGLVQPGGSLRLSCAASGFTFSSYAMNWVRQAPGKGLEWVSIISGTIGKTYYADSVKGRFTISRDNSKNTLYLQMNSLRGEDTAVYYCAKDTERDITGSPFDYWGQGTLVTVSS

SEQ ID NO: 3 - DNA, 24 bp, synthetic construct
  source 1..24: mol_type = other DNA
ggattcacctttagcagctatgcc

SEQ ID NO: 4 - protein, 8 aa, synthetic construct
GFTFSSYA

SEQ ID NO: 5 - DNA, 24 bp, synthetic construct
  source 1..24: mol_type = other DNA
attagtggtactattggtaagaca

SEQ ID NO: 6 - protein, 8 aa, synthetic construct
ISGTIGKT

SEQ ID NO: 7 - DNA, 45 bp, synthetic construct
  source 1..45: mol_type = other DNA
gcgaaagatacggaaagggatataactggatctccttttgactac

SEQ ID NO: 8 - protein, 15 aa, synthetic construct
AKDTERDITGSPFDY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 1347 bp, synthetic construct
  source 1..1347: mol_type = other DNA
gaggtgcagctggtggagtctgggggaggcttggtccagcctggggggtccctgagactctcctgtgcagcctctggattcacctttagcagctatgccatgaactgggtccgccaggctccagggaaggggctggagtgggtctcaattattagtggtactattggtaagacatactacgcagactccgtgaagggccggttcaccatctctagagacaattccaagaacacgctctatctgcaaatgaacagcctgagaggcgaggacacggccgtttattactgtgcgaaagatacggaaagggatataactggatctccttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 448 aa, synthetic construct
EVQLVESGGGLVQPGGSLRLSCAASGFTFSSYAMNWVRQAPGKGLEWVSIISGTIGKTYYADSVKGRFTISRDNSKNTLYLQMNSLRGEDTAVYYCAKDTERDITGSPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 366 bp, synthetic construct
  source 1..366: mol_type = other DNA
cagctgcagctgcaggagtcgggcccaggactggtgaggccttcggagaccctgtcactcacttgtagtgtctctggtgcctccatgaacactaataattactattggggctggatccgccagccccccgggaagggtctggagtggattgggagtggctattataatgggaacacctactataatccgtctctcaagagtcgagtcaccatatctgttgacacgtccaagaaccagttctccctgaagttgaattctgtgaccgccgcagatacggctgtgtattattgtgcgagacagaccccccctcgtataattggaagtccgaatatctggggccaagggacaatggtcaccgtctcttca

SEQ ID NO: 22 - protein, 122 aa, synthetic construct
QLQLQESGPGLVRPSETLSLTCSVSGASMNTNNYYWGWIRQPPGKGLEWIGSGYYNGNTYYNPSLKSRVTISVDTSKNQFSLKLNSVTAADTAVYYCARQTPPRIIGSPNIWGQGTMVTVSS

SEQ ID NO: 23 - DNA, 30 bp, synthetic construct
  source 1..30: mol_type = other DNA
ggtgcctccatgaacactaataattactat

SEQ ID NO: 24 - protein, 10 aa, synthetic construct
GASMNTNNYY

SEQ ID NO: 25 - DNA, 21 bp, synthetic construct
  source 1..21: mol_type = other DNA
ggctattataatgggaacacc

SEQ ID NO: 26 - protein, 7 aa, synthetic construct
GYYNGNT

SEQ ID NO: 27 - DNA, 42 bp, synthetic construct
  source 1..42: mol_type = other DNA
gcgagacagaccccccctcgtataattggaagtccgaatatc

SEQ ID NO: 28 - protein, 14 aa, synthetic construct
ARQTPPRIIGSPNI

SEQ ID NO: 29 - DNA, 1347 bp, synthetic construct
  source 1..1347: mol_type = other DNA
cagctgcagctgcaggagtcgggcccaggactggtgaggccttcggagaccctgtcactcacttgtagtgtctctggtgcctccatgaacactaataattactattggggctggatccgccagccccccgggaagggtctggagtggattgggagtggctattataatgggaacacctactataatccgtctctcaagagtcgagtcaccatatctgttgacacgtccaagaaccagttctccctgaagttgaattctgtgaccgccgcagatacggctgtgtattattgtgcgagacagaccccccctcgtataattggaagtccgaatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 30 - protein, 448 aa, synthetic construct
QLQLQESGPGLVRPSETLSLTCSVSGASMNTNNYYWGWIRQPPGKGLEWIGSGYYNGNTYYNPSLKSRVTISVDTSKNQFSLKLNSVTAADTAVYYCARQTPPRIIGSPNIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 372 bp, synthetic construct
  source 1..372: mol_type = other DNA
gaggtgcagctggtggagtctgggggaggcttggtacagccgggggggtccctgagactctcctgtgcagcctctggattcacctttggcagctatggcatggcgtgggtccgccaggctccagggaaggggctggagtgggtctcagctattagtggtagtggtgattatacaaactacgcagactccgtgaagcgccggttcaccatctccagagacaattccaagaacacgctttatctacaaatgagcagcctgagagccgaggacacggccgtatattactgtgcgaaagagaagactggaacgacgggctatttgtactccggtatggacgtctggggccaagggaccacggtcaccgtctcctca

SEQ ID NO: 32 - protein, 124 aa, synthetic construct
EVQLVESGGGLVQPGGSLRLSCAASGFTFGSYGMAWVRQAPGKGLEWVSAISGSGDYTNYADSVKRRFTISRDNSKNTLYLQMSSLRAEDTAVYYCAKEKTGTTGYLYSGMDVWGQGTTVTVSS

SEQ ID NO: 33 - DNA, 24 bp, synthetic construct
  source 1..24: mol_type = other DNA
ggattcacctttggcagctatggc

SEQ ID NO: 34 - protein, 8 aa, synthetic construct
GFTFGSYG

SEQ ID NO: 35 - DNA, 24 bp, synthetic construct
  source 1..24: mol_type = other DNA
attagtggtagtggtgattataca

SEQ ID NO: 36 - protein, 8 aa, synthetic construct
ISGSGDYT

SEQ ID NO: 37 - DNA, 51 bp, synthetic construct
  source 1..51: mol_type = other DNA
gcgaaagagaagactggaacgacgggctatttgtactccggtatggacgtc

SEQ ID NO: 38 - protein, 17 aa, synthetic construct
AKEKTGTTGYLYSGMDV

SEQ ID NO: 39 - DNA, 1353 bp, synthetic construct
  source 1..1353: mol_type = other DNA
gaggtgcagctggtggagtctgggggaggcttggtacagccgggggggtccctgagactctcctgtgcagcctctggattcacctttggcagctatggcatggcgtgggtccgccaggctccagggaaggggctggagtgggtctcagctattagtggtagtggtgattatacaaactacgcagactccgtgaagcgccggttcaccatctccagagacaattccaagaacacgctttatctacaaatgagcagcctgagagccgaggacacggccgtatattactgtgcgaaagagaagactggaacgacgggctatttgtactccggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 40 - protein, 450 aa, synthetic construct
EVQLVESGGGLVQPGGSLRLSCAASGFTFGSYGMAWVRQAPGKGLEWVSAISGSGDYTNYADSVKRRFTISRDNSKNTLYLQMSSLRAEDTAVYYCAKEKTGTTGYLYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 1347 bp, synthetic construct
  source 1..1347: mol_type = other DNA
cagctgcagctgcaggagtcgggcccaggactggtgaggccttcggagaccctgtcactcacttgtagtgtctctggtgcctccatgaacactaataattactattggggctggatccgccagccccccgggaagggtctggagtggattgggagtggctattataatgggaacacctactataatccgtctctcaagagtcgagtcaccatatctgttgacacgtccaagaaccagttctccctgaagttgaattctgtgaccgccgcagatacggctgtgtattattgtgcgagacagaccccccctcgtataattggaagtccgaatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 42 - protein, 448 aa, synthetic construct
QLQLQESGPGLVRPSETLSLTCSVSGASMNTNNYYWGWIRQPPGKGLEWIGSGYYNGNTYYNPSLKSRVTISVDTSKNQFSLKLNSVTAADTAVYYCARQTPPRIIGSPNIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - DNA, 1353 bp, synthetic construct
  source 1..1353: mol_type = other DNA
gaggtgcagctggtggagtctgggggaggcttggtacagccgggggggtccctgagactctcctgtgcagcctctggattcacctttggcagctatggcatggcgtgggtccgccaggctccagggaaggggctggagtgggtctcagctattagtggtagtggtgattatacaaactacgcagactccgtgaagcgccggttcaccatctccagagacaattccaagaacacgctttatctacaaatgagcagcctgagagccgaggacacggccgtatattactgtgcgaaagagaagactggaacgacgggctatttgtactccggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctggcggtggcggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 44 - protein, 450 aa, synthetic construct
EVQLVESGGGLVQPGGSLRLSCAASGFTFGSYGMAWVRQAPGKGLEWVSAISGSGDYTNYADSVKRRFTISRDNSKNTLYLQMSSLRAEDTAVYYCAKEKTGTTGYLYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 45 - DNA, 1347 bp, synthetic construct
  source 1..1347: mol_type = other DNA
gaggtgcagctggtggagtctgggggaggcttggtccagcctggggggtccctgagactctcctgtgcagcctctggattcacctttagcagctatgccatgaactgggtccgccaggctccagggaaggggctggagtgggtctcaattattagtggtactattggtaagacatactacgcagactccgtgaagggccggttcaccatctctagagacaattccaagaacacgctctatctgcaaatgaacagcctgagaggcgaggacacggccgtttattactgtgcgaaagatacggaaagggatataactggatctccttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 46 - protein, 448 aa, synthetic construct
EVQLVESGGGLVQPGGSLRLSCAASGFTFSSYAMNWVRQAPGKGLEWVSIISGTIGKTYYADSVKGRFTISRDNSKNTLYLQMNSLRGEDTAVYYCAKDTERDITGSPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - DNA, 1347 bp, synthetic construct; identical to SEQ ID NO: 29
  source 1..1347: mol_type = other DNA

SEQ ID NO: 48 - protein, 448 aa, synthetic construct; identical to SEQ ID NO: 30

SEQ ID NO: 49 - DNA, 1353 bp, synthetic construct
  source 1..1353: mol_type = other DNA
gaggtgcagctggtggagtctgggggaggcttggtacagccgggggggtccctgagactctcctgtgcagcctctggattcacctttggcagctatggcatggcgtgggtccgccaggctccagggaaggggctggagtgggtctcagctattagtggtagtggtgattatacaaactacgcagactccgtgaagcgccggttcaccatctccagagacaattccaagaacacgctttatctacaaatgagcagcctgagagccgaggacacggccgtatattactgtgcgaaagagaagactggaacgacgggctatttgtactccggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 50 - protein, 450 aa, synthetic construct
EVQLVESGGGLVQPGGSLRLSCAASGFTFGSYGMAWVRQAPGKGLEWVSAISGSGDYTNYADSVKRRFTISRDNSKNTLYLQMSSLRAEDTAVYYCAKEKTGTTGYLYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 51 - DNA, 1347 bp, synthetic construct; identical to SEQ ID NO: 17
  source 1..1347: mol_type = other DNA

SEQ ID NO: 52 - protein, 448 aa, synthetic construct; identical to SEQ ID NO: 18

SEQ ID NO: 53 - protein, 202 aa, synthetic construct
PEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLREQKLISEEDLGGEQKLISEEDLHHHHHH

SEQ ID NO: 54 - protein, 202 aa, synthetic construct
PEPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQREQKLISEEDLGGEQKLISEEDLHHHHHH

SEQ ID NO: 55 - protein, 202 aa, synthetic construct
LGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMREQKLISEEDLGGEQKLISEEDLHHHHHH

SEQ ID NO: 56 - protein, 407 aa, synthetic construct
PEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - protein, 461 aa, Homo sapiens
  REGION 1..28: note = MISC_FEATURE - Signal peptide
  REGION 29..46: note = MISC_FEATURE - Propeptide
  REGION 47..461: note = MISC_FEATURE - Mature FIX
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 58 - DNA, 2800 bp, Homo sapiens
  source 1..2800: mol_type = other DNA
actttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccgtagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcata

SEQ ID NO: 59 - DNA, 1386 bp, Homo sapiens
  source 1..1386: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 60 - DNA, 1296 bp, synthetic construct
  misc_feature 1..1296: note = Synthetic
  misc_feature 60: note = Cryptic Splice Donor Site Mutated
  misc_feature 201: note = Cryptic Splice Donor Site Mutated
  misc_feature 618: note = Cryptic Splice Donor Site Mutated
  source 1..1296: mol_type = other DNA
tttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaa

SEQ ID NO: 61 - DNA, 1296 bp, synthetic construct
  misc_feature 1..1296: note = Synthetic
  source 1..1296: mol_type = other DNA
ttcctggaccatgagaatgccaacaagatcctcaacagacccaagagatacaacagtggcaagctggaggagtttgtgcagggcaacctggagagggagtgcatggaagagaagtgcagctttgaggaagccagagaagtgtttgaaaacacagagagaaccactgagttctggaagcagtatgttgatggagaccagtgtgagagcaacccttgcctgaatggaggcagctgcaaagatgacatcaacagctatgagtgctggtgcccttttggctttgagggcaagaactgtgagctggatgtcacctgcaacatcaagaatggcagatgtgaacagttctgcaagaactcagcagacaacaaggtggtgtgcagctgcacagagggctacaggctggctgaaaaccagaagagctgtgaacctgctgtccctttcccctgtggcagagtgtctgtgtcccagacctccaagctgaccagggctgaaacagtgttccctgatgtggactatgtgaacagcactgaggctgagaccatcctggacaacatcacccagagcacccagagcttcaatgacttcaccagagtggtgggaggagaggatgccaagcctggccagttcccatggcaggtggtgctgaatggcaaggtggatgccttctgtgggggcagcattgtgaatgaaaaatggattgtgacagctgcccactgtgtggaaacaggtgtcaagatcacagtggtggctggtgaacacaacatagaagaaacagagcacacagagcagaagagaaatgtgatcaggatcatccctcaccacaactacaatgcagccatcaacaagtacaaccatgacattgccctgctggaacttgatgagcctctggtgctgaacagctatgttacccccatctgcattgcagacaaagagtacaccaacatcttcctgaagtttggctctggctatgtgtctggctggggcagagtgttccacaagggcaggtctgccctggtgctgcaatacctgagagtccccctggtggacagagccacctgcctgagaagcaccaagttcaccatctacaacaacatgttctgtgctggcttccatgagggaggcagagacagctgccaaggagactctggaggcccacatgtgacagaggtggagggcaccagcttcctgacaggcatcatcagctggggagaggaatgtgccatgaagggcaagtatggcatctacaccaaggtgtccagatatgtgaactggatcaaagaaaaaaccaaactgacctga

SEQ ID NO: 62 - DNA, 3570 bp, synthetic construct
  misc_feature 1..3570: note = Synthetic
  misc_feature 1..145: note = ITR
  misc_feature 154..253: note = Splice acceptor
  misc_feature 256..1551: note = F9 CDS #1 Native CpG Removed No Splice F9
  misc_feature 315: note = Cryptic splice donor site mutated
  misc_feature 456: note = Cryptic splice donor site mutated
  misc_feature 873: note = Cryptic splice donor site mutated
  misc_feature 1555..1556: note = CpG removed
  misc_feature 1558..1781: note = bGH pA with CpG sites removed
  misc_feature 1792..1793: note = CpG removed
  misc_feature 1889..2019: note = SV40 pA
  misc_feature 2020..3315: note = F9 CDS #2
  misc_feature 3318..3417: note = Splice acceptor
  misc_feature 3426..3570: note = ITR
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 63 - protein, 431 aa, synthetic construct
  REGION 1..431: note = Synthetic
FLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 64 - DNA, 3280 bp, synthetic construct
  misc_feature 1..3280: note = Synthetic
  misc_feature 9..108: note = Splice acceptor
  misc_feature 111..1406: note = F9 CDS #1 Native CpG Removed No Splice F9
  misc_feature 170: note = Cryptic splice donor site mutated
  misc_feature 311: note = Cryptic splice donor site mutated
  misc_feature 728: note = Cryptic splice donor site mutated
  misc_feature 1410..1411: note = CpG removed
  misc_feature 1413..1636: note = bGH pA with CpG sites removed
  misc_feature 1647..1648: note = CpG removed
  misc_feature 1744..1874: note = SV40 pA
  misc_feature 1875..3170: note = F9 CDS #2 (CpG0 F9)
  misc_feature 3173..3272: note = Splice acceptor
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 65 - RNA, 4423 nt, synthetic construct
  misc_feature 1..4423: note = Synthetic
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 66 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 67 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 68 - DNA, 709 bp, Homo sapiens
  source 1..709: mol_type = other DNA
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccag

SEQ ID NO: 69-71 - skipped (3 entries)

SEQ ID NO: 72 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 73 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 74 - DNA, 4218 bp, synthetic construct
  misc_feature 1..4218: note = Synthetic
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 75 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 76 - RNA, 4514 nt, synthetic construct
  misc_feature 1..4514: note = Synthetic
  source 1..4514: mol_type = other RNA
gggtcccgcagtcggcgtccagcggctctgcttgttcgtgtgtgtgtcgttgcaggccttattcggatccatggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatgagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 77 - protein, 7 aa, synthetic construct
  REGION 1..7: note = Synthetic
PKKKRKV

SEQ ID NO: 78 - protein, 7 aa, synthetic construct
  REGION 1..7: note = Synthetic
PKKKRRV

SEQ ID NO: 79 - protein, 16 aa, synthetic construct
  REGION 1..16: note = Synthetic
KRPAATKKAGQAKKKK

SEQ ID NO: 80 - RNA, 16 nt, synthetic construct
  misc_feature 1..16: note = Synthetic
  source 1..16: mol_type = other RNA
gttttagagctatgct

SEQ ID NO: 81 - RNA, 22 nt, synthetic construct
  misc_feature 1..22: note = Synthetic
  source 1..22: mol_type = other RNA
gttttagagctatgctgttttg

SEQ ID NO: 82 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 83 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 84 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 85 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 86 - RNA, 82 nt, synthetic construct; identical to SEQ ID NO: 84
  misc_feature 1..82: note = Synthetic
  source 1..82: mol_type = other RNA

SEQ ID NO: 87 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 88 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 89 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 90 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 91 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 92 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 93 - RNA, 100 nt, synthetic construct
  misc_feature 1..100: note = Synthetic
  source 1..100: mol_type = other RNA
  misc_difference 1..20: note = n is a, c, g, or u
nnnnnnnnnnnnnnnnnnnngttttagagctagaaatagcaagttaaaataaggctagtccgttatcaacttgaaaaagtggcaccgagtcggtgctttt

SEQ ID NO: 94 - RNA, 20 nt, synthetic construct
  misc_feature 1..20: note = Synthetic
  source 1..20: mol_type = other RNA
gagcaacctcactcttgtct

SEQ ID NO: 95 - RNA, 20 nt, synthetic construct
  misc_feature 1..20: note = Synthetic
  source 1..20: mol_type = other RNA
atttatgagatcaacagcac

SEQ ID NO: 96 - RNA, 20 nt, synthetic construct
  misc_feature 1..20: note = Synthetic
  source 1..20: mol_type = other RNA
taaagcatagtgcaatggat

SEQ ID NO: 97 - RNA, 20 nt, synthetic construct
  misc_feature 1..20: note = Synthetic
  source 1..20: mol_type = other RNA
taataaaattcaaacatcct

SEQ ID NO: 98 - RNA, 100 nt, synthetic construct
  misc_feature 1..100: note = Synthetic
  source 1..100: mol_type = other RNA
gagcaacctcactcttgtctgttttagagctagaaatagcaagttaaaataaggctagtccgttatcaacttgaaaaagtggcaccgagtcggtgctttt

SEQ ID NO: 99 - RNA, 100 nt, synthetic construct
  misc_feature 1..100: note = Synthetic
  source 1..100: mol_type = other RNA
atttatgagatcaacagcacgttttagagctagaaatagcaagttaaaataaggctagtccgttatcaacttgaaaaagtggcaccgagtcggtgctttt

SEQ ID NO: 100 - RNA, 100 nt, synthetic construct
  misc_feature 1..100: note = Synthetic
  source 1..100: mol_type = other RNA
taaagcatagtgcaatggatgttttagagctagaaatagcaagttaaaataaggctagtccgttatcaacttgaaaaagtggcaccgagtcggtgctttt

SEQ ID NO: 101 - RNA, 100 nt, synthetic construct
  misc_feature 1..100: note = Synthetic
  source 1..100: mol_type = other RNA
taataaaattcaaacatcctgttttagagctagaaatagcaagttaaaataaggctagtccgttatcaacttgaaaaagtggcaccgagtcggtgctttt

SEQ ID NO: 102 - RNA, 100 nt, synthetic construct; identical to SEQ ID NO: 98
  misc_feature 1..100: note = Synthetic
  source 1..100: mol_type = other RNA

SEQ ID NO: 103 - RNA, 100 nt, synthetic construct; identical to SEQ ID NO: 99
  misc_feature 1..100: note = Synthetic
  source 1..100: mol_type = other RNA

SEQ ID NO: 104 - RNA, 100 nt, synthetic construct; identical to SEQ ID NO: 100
  misc_feature 1..100: note = Synthetic
  source 1..100: mol_type = other RNA

SEQ ID NO: 105 - RNA, 100 nt, synthetic construct; identical to SEQ ID NO: 101
  misc_feature 1..100: note = Synthetic
  source 1..100: mol_type = other RNA

SEQ ID NO: 106 - DNA, 20 bp, synthetic construct
  misc_feature 1..20: note = Synthetic
  source 1..20: mol_type = other DNA
gagcaacctcactcttgtct

SEQ ID NO: 107 - DNA, 20 bp, synthetic construct
  misc_feature 1..20: note = Synthetic
  source 1..20: mol_type = other DNA
atttatgagatcaacagcac

SEQ ID NO: 108 - DNA, 20 bp, synthetic construct
  misc_feature 1..20: note = Synthetic
  source 1..20: mol_type = other DNA
taaagcatagtgcaatggat

SEQ ID NO: 109 - DNA, 20 bp, synthetic construct
  misc_feature 1..20: note = Synthetic
  source 1..20: mol_type = other DNA
taataaaattcaaacatcct

SEQ ID NO: 110 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 111 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 112 - DNA, 130 bp, synthetic construct
  misc_feature 1..130: note = Synthetic
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 113 - DNA, 145 bp, synthetic construct
  misc_feature 1..145: note = Synthetic
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 114 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 115 - DNA, 3751 bp, Homo sapiens
  source 1..3751: mol_type = other DNA
cgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcag

SEQ ID NO: 116 - DNA, 2859 bp, Homo sapiens
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 117 - protein, 883 aa, synthetic construct
  REGION 1..883: note = Synthetic
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 118 - DNA, 2649 bp, synthetic construct
  misc_feature 1..2649: note = Synthetic
  source 1..2649: mol_type = other DNA
gcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 119 - protein, 240 aa, synthetic construct
  REGION 1..240: note = Synthetic
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSSGGSGGGGSGGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 120 - protein, 1128 aa, synthetic construct
  REGION 1..1128: note = Synthetic
  REGION 1..240: note = MISC_FEATURE - 12450 anti-CD63 scFv
  REGION 241..245: note = MISC_FEATURE - G4S linker
  REGION 246..1128: note = MISC_FEATURE - GAA (70-952)
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSSGGSGGGGSGGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 121 - protein, 244 aa, synthetic construct
  REGION 1..244: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 122 - DNA, 732 bp, synthetic construct
  misc_feature 1..732: note = Synthetic
  source 1..732: mol_type = other DNA
gacatccagatgacccagagccccagctccctgtcagccagtgtgggtgacagagtgaccatcacctgcagagccagccagagcatcagcagctacctgaactggtaccagcagaaacctggcaaagcccccaagctgctgatttatgctgctagcagcctgcagagtggagtgcccagcagattctcaggcagtggctctggcacagacttcaccctgaccatctccagcctgcagccagaagactttgccacctactactgccagcagagctacagcacccctcctatcacctttggccagggcacaagactggagatcaagggaggaggaggctctggaggaggaggctctggagggggaggctctgaggtccaactggttgaatctggagggggcctggtgcaacctggcaggagcctgagactgagctgtgctgcctctggcttcacctttgatgactatgccatgaactgggtgagacaggcccctggcaagggcctggagtgggtgtctggcatcagctggagctctggcagcatggactatgcagattctgtgaagggcaggttcaccatcagcagagataatgccaagaacagcctgtacctgcagatgaacagcctcaggacagaggacacagccctgtactactgtgccaaggccagagaagtgggtgactactatggcatggatgtgtggggccagggcaccacagtgacagtgagctct

SEQ ID NO: 123 - DNA, 141 bp, synthetic construct
  misc_feature 1..141: note = Synthetic
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 124 - DNA, 130 bp, synthetic construct
  misc_feature 1..130: note = Synthetic
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 125 - DNA, 3411 bp, synthetic construct
  source 1..3411: mol_type = other DNA
gacatccagatgacccagagccccagctccctgtcagccagtgtgggtgacagagtgaccatcacctgcagagccagccagagcatcagcagctacctgaactggtaccagcagaaacctggcaaagcccccaagctgctgatttatgctgctagcagcctgcagagtggagtgcccagcagattctcaggcagtggctctggcacagacttcaccctgaccatctccagcctgcagccagaagactttgccacctactactgccagcagagctacagcacccctcctatcacctttggccagggcacaagactggagatcaagggaggaggaggctctggaggaggaggctctggagggggaggctctgaggtccaactggttgaatctggagggggcctggtgcaacctggcaggagcctgagactgagctgtgctgcctctggcttcacctttgatgactatgccatgaactgggtgagacaggcccctggcaagggcctggagtgggtgtctggcatcagctggagctctggcagcatggactatgcagattctgtgaagggcaggttcaccatcagcagagataatgccaagaacagcctgtacctgcagatgaacagcctcaggacagaggacacagccctgtactactgtgccaaggccagagaagtgggtgactactatggcatggatgtgtggggccagggcaccacagtgacagtgagctctgggggaggaggctcaggtgggggtggcagtgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgc

SEQ ID NO: 126 - protein, 1137 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 127 - DNA, 2649 bp, synthetic construct
  source 1..2649: mol_type = other DNA
gcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgc

SEQ ID NO: 128 - DNA, 3384 bp, synthetic construct
  source 1..3384: mol_type = other DNA
caagtccagctgcaagagtctggccccaaggtggtcaagccctctgagacactgagcctgacctgcacagtgtctggtggcagcatcagcagctactactggaactggatcagacagagccctggcaaaggactggaatggattggctacaccaagagaggctacacagactacaaccccagcctgagaagcagagtgaccatctctgaggacaccagcaagaaccagttcagcctgaggatcagctctgtgacagctgctgacacagcagtttactactgtgcccagatgggctggggcagccatgcctttgatatgtggggccagggcacaatggtggctgtgtccagtgggggctcaggaggaggtggctcaggaggtggaggttcaggaggggagattgtgctgacccagtctcctggcacactgtctctgagccctggggaaagagccacactgagctgtagagccagccagtctgtgtcctctagctacctggcctggtatcagcagaagcctggacaggctcccagactgctgatctatggggcctcttctagagccacaggcatccctgacagattcagtggcagtggctctggcacagacttcaccctgaccatcagcagactggaacctgaggactttgctgtgtactattgccagcagtatggcagcagcccctggacatttggacagggcaccaaggtggaaatcaaagggggtggtggctctgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgc

SEQ ID NO: 129 - DNA, 720 bp, synthetic construct
  source 1..720: mol_type = other DNA
caagtccagctgcaagagtctggccccaaggtggtcaagccctctgagacactgagcctgacctgcacagtgtctggtggcagcatcagcagctactactggaactggatcagacagagccctggcaaaggactggaatggattggctacaccaagagaggctacacagactacaaccccagcctgagaagcagagtgaccatctctgaggacaccagcaagaaccagttcagcctgaggatcagctctgtgacagctgctgacacagcagtttactactgtgcccagatgggctggggcagccatgcctttgatatgtggggccagggcacaatggtggctgtgtccagtgggggctcaggaggaggtggctcaggaggtggaggttcaggaggggagattgtgctgacccagtctcctggcacactgtctctgagccctggggaaagagccacactgagctgtagagccagccagtctgtgtcctctagctacctggcctggtatcagcagaagcctggacaggctcccagactgctgatctatggggcctcttctagagccacaggcatccctgacagattcagtggcagtggctctggcacagacttcaccctgaccatcagcagactggaacctgaggactttgctgtgtactattgccagcagtatggcagcagcccctggacatttggacagggcaccaaggtggaaatcaaa

SEQ ID NO: 130 - DNA, 4297 bp, synthetic construct
  source 1..4297: mol_type = other DNA
  misc_feature 1..130: note = 130_bp_AAV2_ITR_-_Left
  misc_feature 139..238: note = mAlb_SA
  misc_feature 241..564: note = H1H12847 VK
  misc_feature 565..609: note = 3xG4S
  misc_feature 610..972: note = H1H12847 VH
  misc_feature 973..1002: note = 2xG4S
  misc_feature 1003..3651: note = GAA (70-952)
  misc_feature 3658..3933: note = bGHpA
  misc_feature 3935..4161: note = SV40_late_polyA
  misc_feature 4168..4297: note = 130_bp_AAV2_ITR_-_R
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagttgacatccagatgacccagagccccagctccctgtcagccagtgtgggtgacagagtgaccatcacctgcagagccagccagagcatcagcagctacctgaactggtaccagcagaaacctggcaaagcccccaagctgctgatttatgctgctagcagcctgcagagtggagtgcccagcagattctcaggcagtggctctggcacagacttcaccctgaccatctccagcctgcagccagaagactttgccacctactactgccagcagagctacagcacccctcctatcacctttggccagggcacaagactggagatcaagggaggaggaggctctggaggaggaggctctggagggggaggctctgaggtccaactggttgaatctggagggggcctggtgcaacctggcaggagcctgagactgagctgtgctgcctctggcttcacctttgatgactatgccatgaactgggtgagacaggcccctggcaagggcctggagtgggtgtctggcatcagctggagctctggcagcatggactatgcagattctgtgaagggcaggttcaccatcagcagagataatgccaagaacagcctgtacctgcagatgaacagcctcaggacagaggacacagccctgtactactgtgccaaggccagagaagtgggtgactactatggcatggatgtgtggggccagggcaccacagtgacagtgagctctgggggaggaggctcaggtgggggtggcagtgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgctaataacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccagacatgataagatacattgatgagtttggacaaaccacaactagaatgcagtgaaaaaaatgcttgatttgtgaaatttgtgatgctattgcttgatttgtaaccattataagctgcaataaacaagttaacaacaacaattgcattcattttatgtttcaggttcagggggaggtgtgggaggttttttaaagcaagtaaaacctctacaaatgtggtatggctgaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 131 - DNA, 4122 bp, synthetic construct
  source 1..4122: mol_type = other DNA
  misc_feature 1..130: note = 130_bp_AAV2_ITR_-_Left
  misc_feature 139..238: note = mAlb SA
  misc_feature 241..3624: note = 12450 anti-CD63 scFv:GAA
  misc_feature 3631..3857: note = SV40 polyA
  misc_feature 3858..3888: note = MAZ
  misc_feature 3889..3919: note = MAZ
  misc_feature 3925..3955: note = MAZ
  misc_feature 3956..3986: note = MAZ
  misc_feature 3993..4122: note = 130_bp_AAV2_ITR_-_R
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagttcaggtccagctgcaagagtctggccccaaggtggtcaagccctctgagacactgagcctgacctgcacagtgtctggtggcagcatcagcagctactactggaactggatcagacagagccctggcaaaggcctggaatggattggctacaccaagagaggctacacagactacaaccccagcctgagaagcagagtgaccatctctgaggacaccagcaagaaccagttcagcctgaggatcagctctgtgacagctgctgacacagcagtgtactactgtgcccagatgggctggggcagccatgcctttgatatgtggggccagggcacaatggtggctgtgtccagtgggggctcaggaggaggtggctcaggaggtggaggttcaggaggggagattgtgctgacccagtctcctggcacactgtctctgagccctggggaaagagccacactgagctgtagagccagccagtctgtgtcctctagctacctggcctggtatcagcagaagcctggacaggctcccagactgctgatctatggggcctcttctagagccacaggcatccctgacagattcagtggcagtggctctggcacagacttcaccctgaccatcagcagactggaacctgaggactttgctgtgtactattgccagcagtatggcagcagcccctggacatttggacagggcaccaaggtggaaatcaaaggtggtggtggctctgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaaggtggtagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaaggtggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgctaataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcctggccttgggggagggggaggccagaatgactggccttgggggagggggaggccagaatgagttgactggccttgggggagggggaggccagaatgactggccttgggggagggggaggccagaatgaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 132 - DNA, 4270 bp, synthetic construct
  source 1..4270: mol_type = other DNA
  misc_feature 1..130: note = 130bp AAV2 ITR L
  misc_feature 139..238: note = mAlb SA
  misc_feature 241..3624: note = 12450 anti-CD63 scFv:GAA
  misc_feature 3631..3906: note = bGH pA
  misc_feature 3908..4134: note = SV40 late polyA
  misc_feature 4141..4270: note = 130bp AAV2 ITR R
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagttcaagtccagctgcaagagtctggccccaaggtggtcaagccctctgagacactgagcctgacctgcacagtgtctggtggcagcatcagcagctactactggaactggatcagacagagccctggcaaaggactggaatggattggctacaccaagagaggctacacagactacaaccccagcctgagaagcagagtgaccatctctgaggacaccagcaagaaccagttcagcctgaggatcagctctgtgacagctgctgacacagcagtttactactgtgcccagatgggctggggcagccatgcctttgatatgtggggccagggcacaatggtggctgtgtccagtgggggctcaggaggaggtggctcaggaggtggaggttcaggaggggagattgtgctgacccagtctcctggcacactgtctctgagccctggggaaagagccacactgagctgtagagccagccagtctgtgtcctctagctacctggcctggtatcagcagaagcctggacaggctcccagactgctgatctatggggcctcttctagagccacaggcatccctgacagattcagtggcagtggctctggcacagacttcaccctgaccatcagcagactggaacctgaggactttgctgtgtactattgccagcagtatggcagcagcccctggacatttggacagggcaccaaggtggaaatcaaagggggtggtggctctgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgctaataacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccagacatgataagatacattgatgagtttggacaaaccacaactagaatgcagtgaaaaaaatgcttgatttgtgaaatttgtgatgctattgcttgatttgtaaccattataagctgcaataaacaagttaacaacaacaattgcattcattttatgtttcaggttcagggggaggtgtgggaggttttttaaagcaagtaaaacctctacaaatgtggtatggctgaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 133 - DNA, 4023 bp, synthetic construct
  source 1..4023: mol_type = other DNA
  misc_feature 1..100: note = mAlb_SA
  misc_feature 103..426: note = H1H12847 VK
  misc_feature 427..471: note = 3xG4S
  misc_feature 472..834: note = H1H12847 VH
  misc_feature 835..864: note = 2xG4S
  misc_feature 865..3513: note = GAA (70-952)
  misc_feature 3520..3795: note = bGH pA
  misc_feature 3797..4023: note = SV40_late_polyA
taggtcagtgaagagaagaacaaaaagcagcatattacagttagttgtcttcatcaatctttaaatatgttgtgtggtttttctctccctgtttccacagttgacatccagatgacccagagccccagctccctgtcagccagtgtgggtgacagagtgaccatcacctgcagagccagccagagcatcagcagctacctgaactggtaccagcagaaacctggcaaagcccccaagctgctgatttatgctgctagcagcctgcagagtggagtgcccagcagattctcaggcagtggctctggcacagacttcaccctgaccatctccagcctgcagccagaagactttgccacctactactgccagcagagctacagcacccctcctatcacctttggccagggcacaagactggagatcaagggaggaggaggctctggaggaggaggctctggagggggaggctctgaggtccaactggttgaatctggagggggcctggtgcaacctggcaggagcctgagactgagctgtgctgcctctggcttcacctttgatgactatgccatgaactgggtgagacaggcccctggcaagggcctggagtgggtgtctggcatcagctggagctctggcagcatggactatgcagattctgtgaagggcaggttcaccatcagcagagataatgccaagaacagcctgtacctgcagatgaacagcctcaggacagaggacacagccctgtactactgtgccaaggccagagaagtgggtgactactatggcatggatgtgtggggccagggcaccacagtgacagtgagctctgggggaggaggctcaggtgggggtggcagtgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgctaataacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccagacatgataagatacattgatgagtttggacaaaccacaactagaatgcagtgaaaaaaatgcttgatttgtgaaatttgtgatgctattgcttgatttgtaaccattataagctgcaataaacaagttaacaacaacaattgcattcattttatgtttcaggttcagggggaggtgtgggaggttttttaaagcaagtaaaacctctacaaatgtggtatggctga

SEQ ID NO: 134 - DNA, 3996 bp, synthetic construct
  source 1..3996: mol_type = other DNA
  misc_feature 1..100: note = mAlb SA
  misc_feature 103..3486: note = 12450 anti-CD63 scFv:GAA
  misc_feature 3493..3768: note = bGH pA
  misc_feature 3770..3996: note = SV40 late polyA
taggtcagtgaagagaagaacaaaaagcagcatattacagttagttgtcttcatcaatctttaaatatgttgtgtggtttttctctccctgtttccacagttcaagtccagctgcaagagtctggccccaaggtggtcaagccctctgagacactgagcctgacctgcacagtgtctggtggcagcatcagcagctactactggaactggatcagacagagccctggcaaaggactggaatggattggctacaccaagagaggctacacagactacaaccccagcctgagaagcagagtgaccatctctgaggacaccagcaagaaccagttcagcctgaggatcagctctgtgacagctgctgacacagcagtttactactgtgcccagatgggctggggcagccatgcctttgatatgtggggccagggcacaatggtggctgtgtccagtgggggctcaggaggaggtggctcaggaggtggaggttcaggaggggagattgtgctgacccagtctcctggcacactgtctctgagccctggggaaagagccacactgagctgtagagccagccagtctgtgtcctctagctacctggcctggtatcagcagaagcctggacaggctcccagactgctgatctatggggcctcttctagagccacaggcatccctgacagattcagtggcagtggctctggcacagacttcaccctgaccatcagcagactggaacctgaggactttgctgtgtactattgccagcagtatggcagcagcccctggacatttggacagggcaccaaggtggaaatcaaagggggtggtggctctgcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaagggggcagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaagggggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgctaataacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccagacatgataagatacattgatgagtttggacaaaccacaactagaatgcagtgaaaaaaatgcttgatttgtgaaatttgtgatgctattgcttgatttgtaaccattataagctgcaataaacaagttaacaacaacaattgcattcattttatgtttcaggttcagggggaggtgtgggaggttttttaaagcaagtaaaacctctacaaatgtggtatggctga

SEQ ID NO: 135 - protein, 5 aa, synthetic construct
SYGMH

SEQ ID NO: 136 - protein, 17 aa, synthetic construct
VISYEESNRYHADSVKG

SEQ ID NO: 137 - protein, 11 aa, synthetic construct
DGGIAAPGPDY

SEQ ID NO: 138 - protein, 16 aa, synthetic construct
RSSQSLLYSNGYNYLD

SEQ ID NO: 139 - protein, 7 aa, synthetic construct
LGSNRAS

SEQ ID NO: 140 - protein, 9 aa, synthetic construct
MQARQTPFT

SEQ ID NO: 141 - protein, 120 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 142 - protein, 112 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 143 - protein, 450 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 219 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 10 aa, synthetic construct
GFTFSDYGMN

SEQ ID NO: 146 - protein, 17 aa, synthetic construct
YISSGRGNIYYADTVKG

SEQ ID NO: 147 - protein, 7 aa, synthetic construct
SWGYFDV

SEQ ID NO: 148 - protein, 17 aa, synthetic construct
KSSQSLLNRGNQKNYLT

SEQ ID NO: 149 - protein, 7 aa, synthetic construct
WASTRES

SEQ ID NO: 150 - protein, 9 aa, synthetic construct
QNDYTYPLT

SEQ ID NO: 151 - protein, 116 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSS

SEQ ID NO: 152 - protein, 446 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 153 - protein, 220 aa, synthetic construct
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 10 aa, synthetic construct
GGSISSPGYY

SEQ ID NO: 155 - protein, 7 aa, synthetic construct
IYKSGST

SEQ ID NO: 156 - protein, 12 aa, synthetic construct
RPVVRYFGWFDP

SEQ ID NO: 157 - protein, 6 aa, synthetic construct
QGISSA

SEQ ID NO: 158 - skipped

SEQ ID NO: 159 - protein, 8 aa, synthetic construct
QQFNSYPT

SEQ ID NO: 160 - protein, 121 aa, synthetic construct
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 161 - protein, 106 aa, synthetic construct
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIK

SEQ ID NO: 162 - protein, 448 aa, synthetic construct
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 213 aa, synthetic construct
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 7 aa, synthetic construct
GFSLSRY

SEQ ID NO: 165 - protein, 5 aa, synthetic construct
WGGGS

SEQ ID NO: 166 - protein, 5 aa, synthetic construct
TDGDY

SEQ ID NO: 167 - protein, 16 aa, synthetic construct
RSSQSLVNSNGNTYLH

SEQ ID NO: 168 - protein, 7 aa, synthetic construct
KVSNRFS

SEQ ID NO: 169 - protein, 9 aa, synthetic construct
SQSTHVPWT

SEQ ID NO: 170 - protein, 113 aa, synthetic construct
QVKLEESGPGLVAPSQSLSITCTVSGFSLSRYSVYWVRQPPGKGLEWLGMMWGGGSTDYNSALKSRLSISKDTSKSQVFLKMNSLRTDDTAMYYCVRTDGDYWGQGTSVTVSS

SEQ ID NO: 171 - protein, 113 aa, synthetic construct
ELQLTQSPLSLPVSLGDQASISCRSSQSLVNSNGNTYLHWYLQKPGQSPKLLIYKVSNRFSGVPDRFSGSGSGTDFTLKISRVEAEDLGVYFCSQSTHVPWTFGGGTKLEIKR

SEQ ID NO: 172 - protein, 450 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 125 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGTAAGRDYYYYYGMDVWGQGTTVTVSS

SEQ ID NO: 174 - protein, 112 aa, synthetic construct
AIVMTQPPLSSPVTLGQPASISCRSSQSLVHSDGNTYLNWLQQRPGQPPRLLIYKFFRRLSGVPDRFSGSGAGTDFTLKISRVEAEDVGVYYCMQVTQFPHTFGQGTRLEIK

SEQ ID NO: 175 - protein, 455 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGTAAGRDYYYYYGMDVWGQGTTVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 219 aa, synthetic construct
AIVMTQPPLSSPVTLGQPASISCRSSQSLVHSDGNTYLNWLQQRPGQPPRLLIYKFFRRLSGVPDRFSGSGAGTDFTLKISRVEAEDVGVYYCMQVTQFPHTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 9 aa, synthetic construct
YTFTNYWMH

SEQ ID NO: 178 - protein, 17 aa, synthetic construct
YINPSNDYTKYNQKFKD

SEQ ID NO: 179 - protein, 5 aa, synthetic construct
QGFPY

SEQ ID NO: 180 - protein, 10 aa, synthetic construct
SASSSVSYMH

SEQ ID NO: 181 - protein, 7 aa, synthetic construct
DTSKLAS

SEQ ID NO: 182 - protein, 9 aa, synthetic construct
HQLSSDPFT

SEQ ID NO: 183 - protein, 114 aa, synthetic construct
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSS

SEQ ID NO: 184 - protein, 106 aa, synthetic construct
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIK

SEQ ID NO: 185 - protein, 441 aa, synthetic construct
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 186 - protein, 213 aa, synthetic construct
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 16 aa, synthetic construct
RSSQSLANSNGNTYLH

SEQ ID NO: 188 - protein, 113 aa, synthetic construct
QVKLQESGPGLVKPSETLSITCTVSGFSLSRYSVYWIRQPPGKGPEWMGMMWGGGSTDYSTSLKSRLTISKDTSKSQVSLKMNSLRTDDTAMYYCVRTDGDYWGQGTTVTVSS

SEQ ID NO: 189 - protein, 113 aa, synthetic construct
ELQLTQSPLSLPVTLGQPASISCRSSQSLANSNGNTYLHWYLQRPGQSPRLLIYKVSNRFSGVPDRFSGSGSGTDFTLKISRVEAEDVGVYYCSQSTHVPWTFGGGTKLEIKR

SEQ ID NO: 190 - protein, 440 aa, synthetic construct
QVKLQESGPGLVKPSETLSITCTVSGFSLSRYSVYWIRQPPGKGPEWMGMMWGGGSTDYSTSLKSRLTISKDTSKSQVSLKMNSLRTDDTAMYYCVRTDGDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 191 - protein, 439 aa, synthetic construct
QVKLQESGPGLVKPSETLSITCTVSGFSLSRYSVYWIRQPPGKGPEWMGMMWGGGSTDYSTSLKSRLTISKDTSKSQVSLKMNSLRTDDTAMYYCVRTDGDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 192 - protein, 219 aa, synthetic construct
ELQLTQSPLSLPVTLGQPASISCRSSQSLANSNGNTYLHWYLQRPGQSPRLLIYKVSNRFSGVPDRFSGSGSGTDFTLKISRVEAEDVGVYYCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 5 aa, synthetic construct
TFPIE

SEQ ID NO: 194 - protein, 17 aa, synthetic construct
NFHPYNDDTKYNEKFKG

SEQ ID NO: 195 - protein, 8 aa, synthetic construct
RGKLPFDS

SEQ ID NO: 196 - protein, 11 aa, synthetic construct
RASQDISNYLN

SEQ ID NO: 197 - protein, 7 aa, synthetic construct
FTSRLRS

SEQ ID NO: 198 - protein, 9 aa, synthetic construct
QQDRKLPWT

SEQ ID NO: 199 - protein, 117 aa, synthetic construct
EVQLVQSGAEVKKPGASVKVSCKASGYTFTTFPIEWVRQAPGQGLEWMGNFHPYNDDTKYNEKFKGRVTLTADKSTSTAYMELSRLRSEDTAVYYCARRGKLPFDSWGQGTTVTVSS

SEQ ID NO: 200 - protein, 107 aa, synthetic construct
DIQMTQSPSSLSASVGDRVTISCRASQDISNYLNWYQQKPGKVPKLLIYFTSRLRSGVPSRFSGSGSGTDYTLTISSLQPEDVATYYCQQDRKLPWTFGQGTKLEIK

SEQ ID NO: 201 - protein, 443 aa, synthetic construct
EVQLVQSGAEVKKPGASVKVSCKASGYTFTTFPIEWVRQAPGQGLEWMGNFHPYNDDTKYNEKFKGRVTLTADKSTSTAYMELSRLRSEDTAVYYCARRGKLPFDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 202 - protein, 214 aa, synthetic construct
DIQMTQSPSSLSASVGDRVTISCRASQDISNYLNWYQQKPGKVPKLLIYFTSRLRSGVPSRFSGSGSGTDYTLTISSLQPEDVATYYCQQDRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 5 aa, synthetic construct
RSAMG

SEQ ID NO: 204 - protein, 17 aa, synthetic construct
AIGTRGGSTKYADSVKG

SEQ ID NO: 205 - protein, 17 aa, synthetic construct
RGPGYPSAAIFQDEYHY

SEQ ID NO: 206 - protein, 126 aa, synthetic construct
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSS

SEQ ID NO: 207 - protein, 5 aa, synthetic construct
SDTMG

SEQ ID NO: 208 - protein, 16 aa, synthetic construct
SISSRGVREYADSVKG

SEQ ID NO: 209 - protein, 13 aa, synthetic construct
GALGLPGYRPYNN

SEQ ID NO: 210 - protein, 121 aa, synthetic construct
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSS

SEQ ID NO: 211 - protein, 114 aa, synthetic construct
QVQLVQSGAEVKKPGASVKVSCKASGYTFTNYWMHWVRQAPGQRLEWMGYINPSNDYTKYNQKFKDRVTITRDTSASTAYMELSSLRSEDTAVYYCARQGFPYWGQGTLVTVSS

SEQ ID NO: 212 - protein, 106 aa, synthetic construct
EIVLTQSPATLSLSPGERATLSCSASSSVSYMHWYQQKPGQAPRLLIYDTSKLASGIPARFSGSGSGTDFTLTISSLEPEDFAVYYCHQLSSDPFTFGGGTKVEIK

SEQ ID NO: 213 - protein, 114 aa, synthetic construct
QVQLVQSGAEVKKPGASVKVSCKASGYTFTNYWMHWVRQAPGQRLEWMGYINPSNDYTKYNQKFKDRVTITADKSASTAYMELSSLRSEDTAVYYCARQGFPYWGQGTLVTVSS

SEQ ID NO: 214 - protein, 114 aa, synthetic construct
QVQLVQSGAEVKKPGASVKVSCKASGYTFTNYWMHWVRQAPGQRLEWIGYINPSNDYTKYNQKFKDRATLTADTSTNTAYMELSSLRSEDTAVYYCARQGFPYWGQGTLVTVSS

SEQ ID NO: 215 - protein, 106 aa, synthetic construct
DIQMTQSPSTLSASVGDRVTITCSASSSVSYMHWYQQKPGKAPKLLIYDTSKLASGVPARFSGSGSGTEFTLTISSLQPDDFATYYCHQLSSDPFTFGQGTKVEVK

SEQ ID NO: 216 - protein, 114 aa, synthetic construct
QVQLVQSGAEVKKPGSSVKVSCKASGYTFTNYWMHWVRQAPGQGLEWIGYINPSNDYTKYNQKFKDKATITADESTNTAYMELSSLRSEDTAVYYCARQGFPYWGQGTLVTVSS

SEQ ID NO: 217 - protein, 106 aa, synthetic construct
EIVLTQSPATLSLSPGERATLSCSASSSVSYMHWYQQKPGQAPRLLIYDTSKLASGIPARFSGSGSGTDFTLTISSLEPEDFAVYYCHQLSSDPFTFGQGTKLEIK

SEQ ID NO: 218 - protein, 446 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 5 aa, synthetic construct
DYGMH

SEQ ID NO: 220 - protein, 17 aa, synthetic construct
YISSGNRIIYYADTVKG

SEQ ID NO: 221 - protein, 10 aa, synthetic construct
QDGYRYAMDY

SEQ ID NO: 222 - protein, 17 aa, synthetic construct
KSSQSLLNSGNQKNYLT

SEQ ID NO: 223 - protein, 7 aa, synthetic construct
WTSTRES

SEQ ID NO: 224 - protein, 119 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSS

SEQ ID NO: 225 - protein, 113 aa, synthetic construct
DIVMTQSPDSLAVSLGERATMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 226 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 446 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 229 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 220 aa, synthetic construct
DIVMTQSPDSLAVSLGERATMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 119 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSS

SEQ ID NO: 232 - protein, 113 aa, synthetic construct
DIVMTQSPDSLAVSLGEKVTINCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 233 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 446 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 236 - protein, 449 aa, synthetic construct
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 220 aa, synthetic construct
DIVMTQSPDSLAVSLGEKVTINCKSSQSLLNSGNQKNYLTWHQQKPGQPPKLLIYWTSTRESGVPDRFSGSGSGTDFTLTISSLQAEDVAVYYCQNDYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 10 aa, synthetic construct
GFNIKDYYVH

SEQ ID NO: 239 - protein, 17 aa, synthetic construct
RIDPEDGDSKYAPKFQG

SEQ ID NO: 240 - protein, 9 aa, synthetic construct
SYYVGTYGY

SEQ ID NO: 241 - protein, 10 aa, synthetic construct
SASSSVSYML

SEQ ID NO: 242 - protein, 7 aa, synthetic construct
STSNLAS

SEQ ID NO: 243 - protein, 9 aa, synthetic construct
QQRTFYPYT

SEQ ID NO: 244 - protein, 118 aa, synthetic construct
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSS

SEQ ID NO: 245 - protein, 106 aa, synthetic construct
DIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIK

SEQ ID NO: 246 - protein, 448 aa, synthetic construct
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 448 aa, synthetic construct
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 445 aa, synthetic construct
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 249 - protein, 448 aa, synthetic construct
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 213 aa, synthetic construct
DIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 112 aa, synthetic construct
DIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTSVTVSS

SEQ ID NO: 252 - protein, 112 aa, synthetic construct
DAVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIK

SEQ ID NO: 253 - protein, 5 aa, synthetic construct
SYWMH

SEQ ID NO: 254 - protein, 17 aa, synthetic construct
QINPTTGRSQYNEKFKT

SEQ ID NO: 255 - protein, 8 aa, synthetic construct
WGLQPFAY

SEQ ID NO: 256 - protein, 11 aa, synthetic construct
KASQDVSTAVA

SEQ ID NO: 257 - protein, 7 aa, synthetic construct
SASYRYT

SEQ ID NO: 258 - protein, 9 aa, synthetic construct
QQHYSTPWT

SEQ ID NO: 259 - protein, 117 aa, synthetic construct
QVQLVQSGAEVKKPGSSVKVSCKASGYAFTSYWMHWVRQAPGQGLEWMGQINPTTGRSQYNEKFKTRVTITADKSTSTAYMELSSLRSEDTAVYYCARWGLQPFAYWGQGTLVTVSS

SEQ ID NO: 260 - protein, 107 aa, synthetic construct
DIQMTQSPSFLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIK

SEQ ID NO: 261 - protein, 446 aa, synthetic construct
QVQLVQSGAEVKKPGSSVKVSCKASGYAFTSYWMHWVRQAPGQGLEWMGQINPTTGRSQYNEKFKTRVTITADKSTSTAYMELSSLRSEDTAVYYCARWGLQPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 262 - protein, 214 aa, synthetic construct
DIQMTQSPSFLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 17 aa, synthetic construct
QINPSQGRSQYNEKFKT

SEQ ID NO: 264 - protein, 117 aa, synthetic construct
QVQLVQSGAEVKKPGSSVKVSCKASGYAFTSYWMHWVRQAPGQGLEWMGQINPSQGRSQYNEKFKTRVTITADKSTSTAYMELSSLRSEDTAVYYCARWGLQPFAYWGQGTLVTVSS

SEQ ID NO: 265 - protein, 107 aa, synthetic construct
EIVMTQSPATLSVSPGERATLSCKASQDVSTAVAWYQQKPGQAPRLLIYSASYRYTGIPARFSGSGSGTEFTLTISSLQSEDFAVYYCQQHYSTPWTFGGGTKVEIK

SEQ ID NO: 266 - protein, 446 aa, synthetic construct
QVQLVQSGAEVKKPGSSVKVSCKASGYAFTSYWMHWVRQAPGQGLEWMGQINPSQGRSQYNEKFKTRVTITADKSTSTAYMELSSLRSEDTAVYYCARWGLQPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 267 - protein, 214 aa, synthetic construct
EIVMTQSPATLSVSPGERATLSCKASQDVSTAVAWYQQKPGQAPRLLIYSASYRYTGIPARFSGSGSGTEFTLTISSLQSEDFAVYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 113 aa, synthetic construct
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIK

SEQ ID NO: 269 - protein, 10 aa, synthetic construct
GFTFSDYGMH

SEQ ID NO: 270 - protein, 5 aa, synthetic construct
SYYMY

SEQ ID NO: 271 - protein, 17 aa, synthetic construct
EINPSNGDTNFNEKFKS

SEQ ID NO: 272 - protein, 9 aa, synthetic construct
SDGRNDMDS

SEQ ID NO: 273 - protein, 18 aa, synthetic construct
ISCRASQRVSSSTYSYMH

SEQ ID NO: 274 - protein, 7 aa, synthetic construct
YASNLES

SEQ ID NO: 275 - protein, 9 aa, synthetic construct
QHSWEIPPT

SEQ ID NO: 276 - protein, 118 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSS

SEQ ID NO: 277 - protein, 111 aa, synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIK

SEQ ID NO: 278 - protein, 448 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 218 aa, synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 785 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 281 - protein, 7 aa, synthetic construct
WGGGSTD

SEQ ID NO: 282 - DNA, 1353 bp, synthetic construct
  source 1..1353: mol_type = other DNA
gaggtgcagctggtggagtctgggggaggcttggtacagccgggggggtccctgagactctcctgtgcagcctctggattcacctttggcagctatggcatggcgtgggtccgccaggctccagggaaggggctggagtgggtctcagctattagtggtagtggtgattatacaaactacgcagactccgtgaagcgccggttcaccatctccagagacaattccaagaacacgctttatctacaaatgagcagcctgagagccgaggacacggccgtatattactgtgcgaaagagaagactggaacgacgggctatttgtactccggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 283 - protein, 450 aa, synthetic construct
EVQLVESGGGLVQPGGSLRLSCAASGFTFGSYGMAWVRQAPGKGLEWVSAISGSGDYTNYADSVKRRFTISRDNSKNTLYLQMSSLRAEDTAVYYCAKEKTGTTGYLYSGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 284 - DNA, 1347 bp, synthetic construct
  source 1..1347: mol_type = other DNA
gaggtgcagctggtggagtctgggggaggcttggtccagcctggggggtccctgagactctcctgtgcagcctctggattcacctttagcagctatgccatgaactgggtccgccaggctccagggaaggggctggagtgggtctcaattattagtggtactattggtaagacatactacgcagactccgtgaagggccggttcaccatctctagagacaattccaagaacacgctctatctgcaaatgaacagcctgagaggcgaggacacggccgtttattactgtgcgaaagatacggaaagggatataactggatctccttttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctggcggtggcggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 285 - protein, 448 aa, synthetic construct
EVQLVESGGGLVQPGGSLRLSCAASGFTFSSYAMNWVRQAPGKGLEWVSIISGTIGKTYYADSVKGRFTISRDNSKNTLYLQMNSLRGEDTAVYYCAKDTERDITGSPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 286 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaacttttggatgacctgggtccgccaggctccagggaaggggctggagtgggtggccaacatgaaccaagatggaagtgagaaatactatgtggactctgtgaagggccgattcaccatctccagagacaacgccaagagctcactgtatctgcaaatgaacagcctgagagccgaggacacggctgtgtattactgtgcgagagatcgggaatattgtattagtaccagctgctatgatgactttgactactggggccagggaaccctggtcaccgtctcctca

SEQ ID NO: 287 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 288 - DNA, 24 bp, synthetic construct
  source 1..24: mol_type = other DNA
ggattcacctttagtaacttttgg

SEQ ID NO: 289 - protein, 8 aa, synthetic construct
GFTFSNFW

SEQ ID NO: 290 - DNA, 24 bp, synthetic construct
  source 1..24: mol_type = other DNA
atgaaccaagatggaagtgagaaa

SEQ ID NO: 291 - protein, 8 aa, synthetic construct
MNQDGSEK

SEQ ID NO: 292 - DNA, 54 bp, synthetic construct
  source 1..54: mol_type = other DNA
gcgagagatcgggaatattgtattagtaccagctgctatgatgactttgactac

SEQ ID NO: 293 - protein, 18 aa, synthetic construct
ARDREYCISTSCYDDFDY

SEQ ID NO: 294 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcatagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 295 - protein, 108 aa, synthetic construct
DIQMTQSPSSLSASVGDRVTITCRASQSISSYLNWYQQKPGKAPKLLIYAASSLHSGVPSRFSGSGSGTDFTLTISSLQPEDFATYYCQQSYSTPPITFGQGTRLEIK

SEQ ID NO: 296 - DNA, 18 bp, synthetic construct
  source 1..18: mol_type = other DNA
cagagcattagcagctat

SEQ ID NO: 297 - protein, 6 aa, synthetic construct
QSISSY

SEQ ID NO: 298-299 - skipped (2 entries)

SEQ ID NO: 300 - DNA, 30 bp, synthetic construct; identical to SEQ ID NO: 15
  source 1..30: mol_type = other DNA

SEQ ID NO: 301 - protein, 10 aa, synthetic construct
QQSYSTPPIT

SEQ ID NO: 302 - DNA, 324 bp, synthetic construct; identical to SEQ ID NO: 9
  source 1..324: mol_type = other DNA

SEQ ID NO: 303 - protein, 108 aa, synthetic construct; identical to SEQ ID NO: 10

SEQ ID NO: 304 - DNA, 18 bp, synthetic construct
  source 1..18: mol_type = other DNA
cagagcattagcagctat

SEQ ID NO: 305 - protein, 6 aa, synthetic construct
QSISSY

SEQ ID NO: 306-307 - skipped (2 entries)

SEQ ID NO: 308 - DNA, 30 bp, synthetic construct; identical to SEQ ID NO: 15
  source 1..30: mol_type = other DNA

SEQ ID NO: 309 - protein, 10 aa, synthetic construct
QQSYSTPPIT

SEQ ID NO: 310 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 311 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 312 - DNA, 24 bp, synthetic construct
  source 1..24: mol_type = other DNA
gggtttacattcgacgattacagc

SEQ ID NO: 313 - protein, 8 aa, synthetic construct
GFTFDDYS

SEQ ID NO: 314 - DNA, 24 bp, synthetic construct
  source 1..24: mol_type = other DNA
atatcatggaactcaggaagcaag

SEQ ID NO: 315 - protein, 8 aa, synthetic construct
ISWNSGSK

SEQ ID NO: 316 - DNA, 51 bp, synthetic construct
  source 1..51: mol_type = other DNA
gcaaaatacggcagtggttatggcaagttttatcattatggactggacgtg

SEQ ID NO: 317 - protein, 17 aa, synthetic construct
AKYGSGYGKFYHYGLDV

SEQ ID NO: 318 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggaatggacgtgtggggacaagggacaacagtgacagtgagtagc

SEQ ID NO: 319 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 320 - DNA, 24 bp, synthetic construct
  source 1..24: mol_type = other DNA
gggtttacattcgacgattacagc

SEQ ID NO: 321 - protein, 8 aa, synthetic construct
GFTFDDYS

SEQ ID NO: 322 - DNA, 24 bp, synthetic construct
  source 1..24: mol_type = other DNA
atatcatggaactcaggaagcatc

SEQ ID NO: 323 - protein, 8 aa, synthetic construct
ISWNSGSI

SEQ ID NO: 324 - DNA, 51 bp, synthetic construct
  source 1..51: mol_type = other DNA
gcaaaatacggcagtggttatggcaagttttattattatggaatggacgtg

SEQ ID NO: 325 - protein, 17 aa, synthetic construct
AKYGSGYGKFYYYGMDV

SEQ ID NO: 326 - protein, 451 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 327 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 328 - protein, 215 aa, synthetic construct; identical to SEQ ID NO: 20

SEQ ID NO: 329 - protein, 127 aa, synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 330 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 331 - protein, 123 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 332 - protein, 8 aa, synthetic construct
GFTFNDYA

SEQ ID NO: 333 - protein, 8 aa, synthetic construct
ISWNSDSI

SEQ ID NO: 334 - protein, 20 aa, synthetic construct
AKDNHYGSGSYYYYQYGMDV

SEQ ID NO: 335 - protein, 6 aa, synthetic construct
QSVSSN

SEQ ID NO: 336 - skipped

SEQ ID NO: 337 - protein, 9 aa, synthetic construct
QHYINWPLT

SEQ ID NO: 338 - protein, 8 aa, synthetic construct
GFTFDDYT

SEQ ID NO: 339 - protein, 8 aa, synthetic construct
ISWNSGSI

SEQ ID NO: 340 - protein, 16 aa, synthetic construct
AKDNSGYGHYYYGMDV

SEQ ID NO: 341 - protein, 9 aa, synthetic construct
CLPTRHMAC

SEQ ID NO: 342 - protein, 13 aa, synthetic construct
CPAPELLGGPSVF

(19) United States
(12) Patent Application Publication
(10) Pub. No.: US 2025/0276092 A1
(43) Pub. Date: Sep. 4, 2025

(54) METHODS AND COMPOSITIONS FOR RE-DOSING AAV USING ANTI-CD40 ANTAGONISTIC ANTIBODY TO SUPPRESS HOST ANTI-AAV ANTIBODY RESPONSE

(71) Applicant: Regeneron Pharmaceuticals, Inc., Tarrytown, NY (US)

(72) Inventors: Nicholas Giovannone, Palisades, NY (US); Andrew Baik, Scarsdale, NY (US); Rachel Sattler, New York, NY (US); Andre Limnander, New York, NY (US); Katherine Cygnar, New York, NY (US); Leah Sabin, Goldens Bridge, NY (US); Christos Kyratsous, Irvington, NY (US); Elena S. Garnova, Rye, NY (US); Vishal Kamat, Northbridge, MA (US); Jee Hae Kim, Ardsley, NY (US); Jamie M. Orengo, Cortlandt Manor, NY (US); Ashique Rafique, Yonkers, NY (US); Changhyun Seong, Mount Kisco, NY (US); Carley C. Tasker, New York, NY (US)

(21) Appl. No.: 19/067,313

(22) Filed: Feb. 28, 2025

Related U.S. Application Data
(60) Provisional application No. 63/733,542, filed on Dec. 13, 2024.
(60) Provisional application No. 63/660,014, filed on Jun. 14, 2024.
(60) Provisional application No. 63/639,227, filed on Apr. 26, 2024.
(60) Provisional application No. 63/560,295, filed on Mar. 1, 2024.

(51) Int. Cl. A61K 48/00; A61K 9/1272; A61K 9/51; A61K 38/37; A61K 38/47; A61K 38/48; A61P 7/04; C07K 16/28; C12N 9/22; C12N 15/11; C12N 15/86; C12N 15/88; C12N 15/90

(52) U.S. Cl. CPC A61K 48/005; A61K 9/1272; A61K 9/5123; A61K 38/37; A61K 38/47; A61K 38/4846; A61K 48/0083; A61P 7/04; C07K 16/2809; C07K 16/283; C07K 16/2875; C07K 16/2878; C07K 16/2881; C07K 16/2887; C07K 16/2896; C12N 9/22; C12N 15/111; C12N 15/86; C12N 15/88; C12N 15/907; C12Y 302/0102; C12Y 304/21022; C07K 2317/31; C07K 2317/52; C07K 2317/565; C07K 2317/71; C07K 2317/76; C07K 2317/92; C12N 2310/20; C12N 2750/14143; C12N 2830/50

(57) ABSTRACT

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency in a subject in need thereof, and methods of preventing or reducing the onset of a sign or symptom of an enzyme deficiency in a subject in need thereof. The methods use CD40 inhibitors (e.g., CD40 antigen-binding molecules) to mitigate immune response and facilitate redosing of nucleic acid constructs encoding a polypeptide of interest and nuclease agents targeting a target genomic locus to achieve, for example, a step-wise increase in expression of a polypeptide of interest in a subject following insertion of the nucleic acid construct without overshooting.

### CROSS-REFERENCE TO RELATED APPLICATIONS

This application claims the benefit of U.S. Application No. 63/560,295, filed Mar. 1, 2024, U.S. Application No. 63/639,227, filed Apr. 26, 2024, U.S. Application No. 63/660,014, filed Jun. 14, 2024, and U.S. Application No. 63/733,542, filed Dec. 13, 2024, each of which is herein incorporated by reference in its entirety for all purposes.

### REFERENCE TO A SEQUENCE LISTING SUBMITTED AS AN XML FILE

The Sequence Listing written in file 625781SEQLIST.xml is 477,278 bytes, was created on Feb. 28, 2025, and is hereby incorporated by reference in its entirety.

### BACKGROUND

CD40 is a cell surface receptor that is part of the tumor necrosis factor (TNF) receptor superfamily. CD40 is expressed on antigen-presenting cells such as B cells, macrophages, and dendritic cells, as well as some non-immune cells and tumors. The interaction of CD40 with its ligand CD40L provides a co-stimulatory signal that is essential to the survival of many cell types and is required for functions of immune response such as B cell activation (particularly in response to T-dependent antigens), germinal center formation and selection, development of long-lived plasma cells and memory B cells responses, IgG class switching, and “licensing” of dendritic cells to mature and become more potent inducers of T-cell immunity.

In AAV gene therapies, seronegative/naive patients are dosed with AAV and develop antibody responses to the AAV capsid antigen. This antibody response prevents future re-dosing of AAV because the antibodies are neutralizing, and the antibody response is sustained for 10+ years.

### SUMMARY

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency in a subject in need thereof, and methods of preventing or reducing the onset of a sign or symptom of an enzyme deficiency in a subject in need thereof. Also provided are compositions, combinations, or kits, e.g., for use in such methods.

In one aspect, provided are methods of inserting a nucleic acid encoding a polypeptide of interest into a target genomic locus in a cell or a population of cells in a subject. Such methods can comprise administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) a CD40 inhibitor (e.g., CD40 antigen-binding molecule), wherein the nuclease agent cleaves the nuclease target site, and the nucleic acid construct is inserted into the target genomic locus. In another aspect, provided are methods of expressing a polypeptide of interest from a target genomic locus in a cell or a population of cells in a subject. Such methods can comprise administering to the subject: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, (c) a CD40 inhibitor (e.g., CD40 antigen-binding molecule), wherein the nuclease agent cleaves the nuclease target site, the nucleic acid construct is inserted into the target genomic locus to create a modified target genomic locus, and the polypeptide of interest is expressed from the modified target genomic locus. In another aspect, provided are methods of treating an enzyme deficiency in a subject in need thereof. Such methods can comprise administering to the subject: (a) a nucleic acid construct comprising a coding sequence for a polypeptide of interest, wherein the polypeptide of interest comprises an enzyme to treat the enzyme deficiency; (b) a nuclease agent or one or more nucleic acids encoding the nuclease agent, wherein the nuclease agent targets a nuclease target site in a target genomic locus; and (c) a CD40 inhibitor (e.g., CD40 antigen-binding molecule), wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency. In another aspect, provided are methods of preventing or reducing the onset of a sign or symptom of an enzyme deficiency in a subject in need thereof. Such methods can comprise administering to the subject: (a) a nucleic acid construct comprising a coding sequence for a polypeptide of interest, wherein the enzyme deficiency is characterized by a loss-of-function of the polypeptide of interest; (b) a nuclease agent or one or more nucleic acids encoding the nuclease agent, wherein the nuclease agent targets a nuclease target site in a target genomic locus; and (c) a CD40 inhibitor (e.g., CD40 antigen-binding molecule), wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency. In some such methods, the subject has a disease of a bleeding disorder characterized by the enzyme deficiency, a disease of an inborn error of metabolism characterized by the enzyme deficiency, or a lysosomal storage disease characterized by the enzyme deficiency. In some such methods, wherein the disease is hemophilia B and the polypeptide of interest is a factor IX protein, the disease is hemophilia A and the polypeptide of interest is a factor VIII protein, or the disease is Pompe disease and the polypeptide of interest is a multidomain therapeutic protein comprising a delivery domain fused to a lysosomal alpha-glucosidase. In some such methods, the subject does not have preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent (e.g., at the time the CD40 inhibitor is administered).

In some such methods, the method further comprises a subsequent administration step. In some such methods, the further administration step comprises administering to the subject at one or more subsequent times: (a) the nucleic acid construct; (b) the nuclease agent or the one or more nucleic acids encoding the nuclease agent; and optionally (c) the CD40 inhibitor (e.g., CD40 antigen-binding molecule), until a desired level of expression and/or activity of the polypeptide of interest is achieved in the subject. In some such methods, the further administration step comprises administering to the subject at one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the CD40 inhibitor (e.g., CD40 antigen-binding molecule), until a desired level of expression and/or activity of the polypeptide of interest is achieved in the subject. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In some such methods, the further administration step comprises administering to the subject at one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor (e.g., CD40 antigen-binding molecule), until a desired level of expression and/or activity of the polypeptide of interest is achieved in the subject. In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some such methods, the further administration step comprises administering to the subject at one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor (e.g., CD40 antigen-binding molecule), until a desired level of expression and/or activity of the polypeptide of interest is achieved in the subject. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some such methods, the method comprises the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest is achieved in the subject. In some such methods, the dose of the nucleic acid construct for the subsequent administration step is higher than the dose of the nucleic acid construct in the first administration step and/or the dose of the nuclease agent or one or more nucleic acids encoding the nuclease agent for the subsequent step is higher than the dose of the nuclease agent or one or more nucleic acids encoding the nuclease agent in the first administration step. In some such methods, the dose of the nucleic acid construct for the subsequent administration step is higher than the dose of the nucleic acid construct in the first administration step. In some such methods, the dose of the nucleic acid construct for the subsequent administration step is lower than the dose of the nucleic acid construct in the first administration step and/or the dose of the nuclease agent or one or more nucleic acids encoding the nuclease agent for the subsequent step is lower than the dose of the nuclease agent or one or more nucleic acids encoding the nuclease agent in the first administration step. In some such methods, the dose of the nucleic acid construct for the subsequent administration step is lower than the dose of the nucleic acid construct in the first administration step. In some such methods, the dose of the nucleic acid construct for the subsequent administration step is the same as the dose of the nucleic acid construct in the first administration step and/or the dose of the nuclease agent or one or more nucleic acids encoding the nuclease agent for the subsequent step is the same as the dose of the nuclease agent or one or more nucleic acids encoding the nuclease agent in the first administration step. In some such methods, the dose of the nucleic acid construct for the subsequent administration step is the same as the dose of the nucleic acid construct in the first administration step. In some such methods, the polypeptide of interest is a factor IX protein, and the desired expression level of the factor IX protein in the subject is a serum level of at least about 3 μg/mL or about 3-5 μg/mL. In some such methods, the polypeptide of interest is a multidomain therapeutic protein comprising a delivery domain fused to a lysosomal alpha-glucosidase, and the desired expression level of the multidomain therapeutic protein in the subject is a serum level of at least about 2 μg/mL or at least about 5 μg/mL. In some such methods, the further administration step comprises administering to the subject at one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor (e.g., CD40 antigen-binding molecule), wherein the second nuclease agent cleaves the second nuclease target site, and the second nucleic acid construct is inserted into the second target genomic locus. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some such methods, the one or more subsequent administration steps is one subsequent administration step. In some such methods, the one or more subsequent administration steps is two subsequent administration steps or comprises at least two subsequent administration steps. In some such methods, the CD40 inhibitor (e.g., CD40 antigen-binding molecule) is administered in the one or more subsequent administration steps if there is no preexisting CD40 inhibitor (e.g., CD40 antigen-binding molecule) in the subject or if preexisting CD40 inhibitor (e.g., CD40 antigen-binding molecule) expression and/or activity levels are below a desired threshold level, In some such methods, the method comprises measuring CD40 inhibitor (e.g., CD40 antigen-binding molecule) expression and/or activity levels prior to the one or more subsequent administration steps.

In some such methods, the CD40 inhibitor is a CD40 antigen-binding molecule. In some such methods, the CD40 antigen-binding molecule is a monospecific CD40 antigen-binding molecule. In some such methods, the CD40 antigen-binding molecule is a bispecific antigen-binding molecule comprising: (a) a first antigen-binding domain (D1) that binds a first epitope of human CD40; and (b) a second antigen-binding domain (D2) that binds a second epitope of human CD40. In some such methods, the bispecific antigen-binding molecule: (i) binds human CD40 with a K_{D} of less than 25 nM as measured by surface plasmon resonance at 25° C.; (ii) binds human CD40 with a K_{D} of less than 70 nM as measured by surface plasmon resonance at 37° C.; (iii) binds human CD40 with a dissociative half-life (t½) of greater than 75 minutes as measured by surface plasmon resonance at 25° C.; (iv) binds a human CD40-expressing cell with an EC₅₀ value of about 10 nM or less; (v) inhibits binding of human CD40 dimer to CD40L; (vi) inhibits CD40 ligand (CD40L)-induced activation; and/or (vii) does not significantly agonize CD40 in the absence of CD40L. In some such methods, the bispecific antigen-binding molecule: (i) binds human CD40 with a K_{D} of less than 25 nM as measured by surface plasmon resonance at 25° C.; (ii) binds human CD40 with a K_{D} of less than 70 nM as measured by surface plasmon resonance at 37° C.; (iii) binds human CD40 with a dissociative half-life (t½) of greater than 75 minutes as measured by surface plasmon resonance at 25° C.; (iv) binds a human CD40-expressing cell with an EC₅₀ value of about 10 nM or less; (v) inhibits binding of human CD40 monomer to CD40L; (vi) inhibits CD40 ligand (CD40L)-induced activation; and/or (vii) does not significantly agonize CD40 in the absence of CD40L. In some such methods, the bispecific antigen-binding molecule inhibits CD40L-induced activation. In some such methods, the bispecific antigen-binding molecule inhibits CD40L-induced activation and does not significantly agonize CD40 in the absence of CD40L. In some such methods, the D1 domain and the D2 domain each comprise a heavy chain immunoglobulin variable region comprising a set of three heavy chain complementarity determining region sequences HCDR1, HCDR2, and HCDR3 independently selected from the group consisting of: (a) an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; (b) an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28; and (c) an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38. In some such methods, the D1 domain and the D2 domain each comprise a light chain immunoglobulin variable region comprising a set of three light chain complementarity determining region sequences LCDR1, LCDR2, and LCDR3, wherein the LCDR1 comprises the amino acid sequence of SEQ ID NO: 12, the LCDR2 comprises the amino acid sequence AAS (SEQ ID NO: 14), and the LCDR3 comprises the amino acid sequence of SEQ ID NO: 16.

In some such methods, the D1 domain comprises: (a) an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; or (b) an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 domain comprises an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 domain comprises a heavy chain variable region (HCVR) comprising the amino acid sequence of SEQ ID NO: 2. In some such methods, the D1 domain comprises an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 domain comprises an HCVR comprising the amino acid sequence of SEQ ID NO: 22.

In some such methods, the D1 domain comprises a light chain variable region (LCVR) comprising the amino acid sequence of SEQ ID NO: 10. In some such methods, the D2 domain comprises an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D2 domain comprises an HCVR comprising the amino acid sequence of SEQ ID NO: 32. In some such methods, the D2 domain comprises an LCVR comprising the amino acid sequence of SEQ ID NO: 10.

In some such methods, the bispecific antigen-binding molecule comprises: a D1 comprising an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 comprises an HCVR comprising the amino acid sequence of SEQ ID NO: 2 and an LCVR comprising the amino acid sequence of SEQ ID NO: 10, and the D2 comprises an HCVR comprising the amino acid sequence of SEQ ID NO: 32 and an LCVR comprising the amino acid sequence of SEQ ID NO: 10.

In some such methods, the bispecific antigen-binding molecule comprises: a D1 comprising an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 comprises an HCVR comprising the amino acid sequence of SEQ ID NO: 22 and an LCVR comprising the amino acid sequence of SEQ ID NO: 10, and the D2 comprises an HCVR comprising the amino acid sequence of SEQ ID NO: 32 and an LCVR comprising the amino acid sequence of SEQ ID NO: 10.

In some such methods, the D1 domain comprises an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D1 domain comprises an HCVR comprising the amino acid sequence of SEQ ID NO: 32. In some such methods, the D1 domain comprises an LCVR comprising the amino acid sequence of SEQ ID NO: 10.

In some such methods, the D2 domain comprises an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such methods, the D2 domain comprises an HCVR comprising the amino acid sequence of SEQ ID NO: 2. In some such methods, the D2 domain comprises an LCVR comprising the amino acid sequence of SEQ ID NO: 10.

In some such methods, the bispecific antigen-binding molecule comprises: a D1 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some such methods, the D1 comprises an HCVR comprising the amino acid sequence of SEQ ID NO: 32 and an LCVR comprising the amino acid sequence of SEQ ID NO: 10, and the D2 comprises an HCVR comprising the amino acid sequence of SEQ ID NO: 2 and an LCVR comprising the amino acid sequence of SEQ ID NO: 10.

In some such methods, the bispecific antigen-binding molecule is a bispecific antibody. In some such methods, the bispecific antibody comprises a human IgG heavy chain constant region. In some such methods, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such methods, the human IgG heavy chain constant region is isotype IgG4 or IgG1. In some such methods, the human IgG heavy chain constant region comprises one or more modifications in a hinge region. In some such methods, the human IgG heavy chain constant region comprises one or more modifications that reduce binding to an Fc receptor (e.g., one or more modifications in a hinge region and/or CH region).

In some such methods, the D1 comprises: a heavy chain comprising the amino acid sequence of SEQ ID NO: 42 and a light chain comprising the amino acid sequence of SEQ ID NO: 20; or a heavy chain comprising the amino acid sequence of SEQ ID NO: 46 and a light chain comprising the amino acid sequence of SEQ ID NO: 20; or a heavy chain comprising the amino acid sequence of SEQ ID NO: 48 and a light chain comprising the amino acid sequence of SEQ ID NO: 20; or a heavy chain comprising the amino acid sequence of SEQ ID NO: 52 and a light chain comprising the amino acid sequence of SEQ ID NO: 20. In some such methods, the D1 comprises: a heavy chain comprising the amino acid sequence of SEQ ID NO: 283 and a light chain comprising the amino acid sequence of SEQ ID NO: 20. In some such methods, the D2 comprises: a heavy chain comprising the amino acid sequence of SEQ ID NO: 44 and a light chain comprising the amino acid sequence of SEQ ID NO: 20; or a heavy chain comprising the amino acid sequence of SEQ ID NO: 50 and a light chain comprising the amino acid sequence of SEQ ID NO: 20. In some such methods, the D2 comprises: a heavy chain comprising the amino acid sequence of SEQ ID NO: 285 and a light chain comprising the amino acid sequence of SEQ ID NO: 20.

In some such methods, the bispecific antigen-binding molecule is a bispecific antibody comprising (i) a D1 comprising a heavy chain comprising the amino acid sequence of SEQ ID NO: 42 and a light chain comprising the amino acid sequence of SEQ ID NO: 20; and (ii) a D2 comprising a heavy chain comprising the amino acid sequence of SEQ ID NO: 44 and a light chain comprising the amino acid sequence of SEQ ID NO: 20. In some such methods, the bispecific antigen-binding molecule is a bispecific antibody comprising (i) a D1 comprising a heavy chain comprising the amino acid sequence of SEQ ID NO: 46 and a light chain comprising the amino acid sequence of SEQ ID NO: 20; and (ii) a D2 comprising a heavy chain comprising the amino acid sequence of SEQ ID NO: 44 and a light chain comprising the amino acid sequence of SEQ ID NO: 20. In some such methods, the bispecific antigen-binding molecule is a bispecific antibody comprising (i) a D1 comprising a heavy chain comprising the amino acid sequence of SEQ ID NO: 48 and a light chain comprising the amino acid sequence of SEQ ID NO: 20; and (ii) a D2 comprising a heavy chain comprising the amino acid sequence of SEQ ID NO: 50 and a light chain comprising the amino acid sequence of SEQ ID NO: 20. In some such methods, the bispecific antigen-binding molecule is a bispecific antibody comprising (i) a D1 comprising a heavy chain comprising the amino acid sequence of SEQ ID NO: 52 and a light chain comprising the amino acid sequence of SEQ ID NO: 20; and (ii) a D2 comprising a heavy chain comprising the amino acid sequence of SEQ ID NO: 50 and a light chain comprising the amino acid sequence of SEQ ID NO: 20.

In some such methods, the D1 comprises a heavy chain comprising the amino acid sequence of SEQ ID NO: 283 and a light chain comprising the amino acid sequence of SEQ ID NO: 20. In some such methods, the D2 comprises a heavy chain comprising the amino acid sequence of SEQ ID NO: 285 and a light chain comprising the amino acid sequence of SEQ ID NO: 20. In some such methods, the bispecific antigen-binding molecule is a bispecific antibody comprising (i) a D1 comprising a heavy chain comprising the amino acid sequence of SEQ ID NO: 283 and a light chain comprising the amino acid sequence of SEQ ID NO: 20; and (ii) a D2 comprising a heavy chain comprising the amino acid sequence of SEQ ID NO: 285 and a light chain comprising the amino acid sequence of SEQ ID NO: 20.

In some such methods, the bispecific antigen-binding molecule is a bispecific antibody having the amino acid sequences of REGN16334. In some such methods, the bispecific antigen-binding molecule is a bispecific antibody having the amino acid sequences of REGN16335. In some such methods, the bispecific antigen-binding molecule is a bispecific antibody having the amino acid sequences of REGN16431. In some such methods, the bispecific antigen-binding molecule is a bispecific antibody having the amino acid sequences of REGN16432. In some such methods, the bispecific antigen-binding molecule is a bispecific antibody having the amino acid sequences of REGN20484.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within an HCVR/LCVR amino acid sequence pair selected from the group consisting of SEQ ID NOS: 2/10, 22/10, 32/10, 183/184, 141/142, 151/268, 231/232, 160/161, 170/171, 173/174, 188/189, 199/200, 183/212, 183/215, 183/217, 211/184, 211/212, 211/215, 211/217, 213/184, 213/212, 213/215, 213/217, 214/184, 214/212, 214/215, 214/217, 216/184, 216/212, 216/215, 216/217, 224/225, 244/245, 251/252, 259/260, 264/265, and 259/265; (II) the HCVR and LCVR amino acid sequences contained within an HCVR/LCVR amino acid sequence pair selected from the group consisting of SEQ ID NOS: 2/10, 22/10, 32/10, 183/184, 141/142, 151/268, 231/232, 160/161, 170/171, 173/174, 188/189, 199/200, 183/212, 183/215, 183/217, 211/184, 211/212, 211/215, 211/217, 213/184, 213/212, 213/215, 213/217, 214/184, 214/212, 214/215, 214/217, 216/184, 216/212, 216/215, 216/217, 224/225, 244/245, 251/252, 259/260, 264/265, and 259/265; or (III) the heavy chain and light chain amino acid sequences contained within a heavy chain/light chain amino acid sequence pair selected from the group consisting of SEQ ID NOS: 18/20, 30/20, 40/20, 185/186, 143/144, 152/153, 236/237, 162/163, 172/144, 175/176, 190/192, 191/192, 201/202, 218/153, 229/230, 233/237, 234/237, 235/237, 246/250, 247/250, 248/250, 249/250, 261/262, 266/267, and 261/267.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within an HCVR/LCVR amino acid sequence pair selected from the group consisting of SEQ ID NOS: 2/10, 22/10, and 32/10; (II) (a) an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; or (b) an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; or (c) an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; (III) (a) an HCVR comprising or consisting of the amino acid sequence of SEQ ID NO: 2 and an LCVR comprising or consisting of the amino acid sequence of SEQ ID NO: 10; (b) an HCVR comprising or consisting of the amino acid sequence of SEQ ID NO: 22 and an LCVR comprising or consisting of the amino acid sequence of SEQ ID NO: 10; (c) an HCVR comprising or consisting of the amino acid sequence of SEQ ID NO: 32 and an LCVR comprising or consisting of the amino acid sequence of SEQ ID NO: 10; or (IV) (a) a heavy chain comprising or consisting of the amino acid sequence of SEQ ID NO: 18 and a light chain comprising or consisting of the amino acid sequence of SEQ ID NO: 20; (b) a heavy chain comprising or consisting of the amino acid sequence of SEQ ID NO: 30 and a light chain comprising or consisting of the amino acid sequence of SEQ ID NO: 20; or (c) a heavy chain comprising or consisting of the amino acid sequence of SEQ ID NO: 40 and a light chain comprising or consisting of the amino acid sequence of SEQ ID NO: 20.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 183/184; (II) an HCDR1 comprising the amino acid sequence of SEQ ID NO: 177, an HCDR2 comprising the amino acid sequence of SEQ ID NO: 178, an HCDR3 comprising the amino acid sequence of SEQ ID NO: 179, an LCDR1 comprising the amino acid sequence of SEQ ID NO: 180, an LCDR2 comprising the amino acid sequence of SEQ ID NO: 181, and an LCDR3 comprising the amino acid sequence of SEQ ID NO: 182; (III) an HCVR comprising or consisting of the amino acid sequence of SEQ ID NO: 183 and an LCVR comprising or consisting of the amino acid sequence of SEQ ID NO: 184; or (IV) a heavy chain comprising or consisting of the amino acid sequence of SEQ ID NO: 185 and a light chain comprising or consisting of the amino acid sequence of SEQ ID NO: 186.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 141/142; (II) an HCDR1 comprising the amino acid sequence of SEQ ID NO: 135, an HCDR2 comprising the amino acid sequence of SEQ ID NO: 136, an HCDR3 comprising the amino acid sequence of SEQ ID NO: 137, an LCDR1 comprising the amino acid sequence of SEQ ID NO: 138, an LCDR2 comprising the amino acid sequence of SEQ ID NO: 139, and an LCDR3 comprising the amino acid sequence of SEQ ID NO: 140; (III) an HCVR comprising or consisting of the amino acid sequence of SEQ ID NO: 141 and an LCVR comprising or consisting of the amino acid sequence of SEQ ID NO: 142; or (IV) a heavy chain comprising or consisting of the amino acid sequence of SEQ ID NO: 143 and a light chain comprising or consisting of the amino acid sequence of SEQ ID NO: 144.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 151/268; (II) an HCDR1 comprising the amino acid sequence of SEQ ID NO: 145, an HCDR2 comprising the amino acid sequence of SEQ ID NO: 146, an HCDR3 comprising the amino acid sequence of SEQ ID NO: 147, an LCDR1 comprising the amino acid sequence of SEQ ID NO: 148, an LCDR2 comprising the amino acid sequence of SEQ ID NO: 149, and an LCDR3 comprising the amino acid sequence of SEQ ID NO: 150; (III) an HCVR comprising or consisting of the amino acid sequence of SEQ ID NO: 151 and an LCVR comprising or consisting of the amino acid sequence of SEQ ID NO: 268; or (IV) a heavy chain comprising or consisting of the amino acid sequence of SEQ ID NO: 152 and a light chain comprising or consisting of the amino acid sequence of SEQ ID NO: 153.

In some such methods, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 231/232; (II) an HCDR1 comprising the amino acid sequence of SEQ ID NO: 269, an HCDR2 comprising the amino acid sequence of SEQ ID NO: 220, an HCDR3 comprising the amino acid sequence of SEQ ID NO: 221, an LCDR1 comprising the amino acid sequence of SEQ ID NO: 222, an LCDR2 comprising the amino acid sequence of SEQ ID NO: 223, and an LCDR3 comprising the amino acid sequence of SEQ ID NO: 150; (III) an HCVR comprising or consisting of the amino acid sequence of SEQ ID NO: 231 and an LCVR comprising or consisting of the amino acid sequence of SEQ ID NO: 232; or (IV) a heavy chain comprising or consisting of the amino acid sequence of SEQ ID NO: 236 and a light chain comprising or consisting of the amino acid sequence of SEQ ID NO: 237.

In some such methods, the CD40 inhibitor is a CD40L binding protein. In some such methods, (I) the CD40L binding protein comprises two Tn3 proteins fused to human serum albumin; or (II) the CD40L binding protein comprises the sequence set forth in SEQ ID NO: 280. In some such methods, the CD40L binding protein is an anti-CD40L antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 276/277; (II) an HCDR1 comprising the amino acid sequence of SEQ ID NO: 270, an HCDR2 comprising the amino acid sequence of SEQ ID NO: 271, an HCDR3 comprising the amino acid sequence of SEQ ID NO: 272, an LCDR1 comprising the amino acid sequence of SEQ ID NO: 273, an LCDR2 comprising the amino acid sequence of SEQ ID NO: 274, and an LCDR3 comprising the amino acid sequence of SEQ ID NO: 275; (III) an HCVR comprising or consisting of the amino acid sequence of SEQ ID NO: 276 and an LCVR comprising or consisting of the amino acid sequence of SEQ ID NO: 277; or (IV) a heavy chain comprising or consisting of the amino acid sequence of SEQ ID NO: 278 and a light chain comprising or consisting of the amino acid sequence of SEQ ID NO: 279.

In some such methods, the CD40 inhibitor (e.g., CD40 antigen-binding molecule) is administered simultaneously with the nucleic acid construct and/or the nuclease agent or the one or more nucleic acids encoding the nuclease agent. In some such methods, the CD40 inhibitor (e.g., CD40 antigen-binding molecule) is administered prior to the nucleic acid construct and/or the nuclease agent or the one or more nucleic acids encoding the nuclease agent. In some such methods, the CD40 inhibitor (e.g., CD40 antigen-binding molecule) is administered prior to and after the nucleic acid construct and/or the nuclease agent or the one or more nucleic acids encoding the nuclease agent. In some such methods, the CD40 inhibitor (e.g., CD40 antigen-binding molecule) is administered within about 6 months after the nucleic acid construct and/or the nuclease agent or the one or more nucleic acids encoding the nuclease agent. In some such methods, the CD40 inhibitor (e.g., CD40 antigen-binding molecule) is administered about 1 week prior to or within about 1 week prior to the nucleic acid construct and/or the nuclease agent or the one or more nucleic acids encoding the nuclease agent.

In some such methods, the CD40 inhibitor (e.g., CD40 antigen-binding molecule) is administered simultaneously with the nucleic acid construct. In some such methods, the CD40 inhibitor (e.g., CD40 antigen-binding molecule) is administered prior to the nucleic acid construct. In some such methods, the CD40 inhibitor (e.g., CD40 antigen-binding molecule) is administered prior to and after the nucleic acid construct. In some such methods, the CD40 inhibitor (e.g., CD40 antigen-binding molecule) is administered within about 6 months after the nucleic acid construct, optionally wherein the nucleic acid construct is in a viral vector, and the CD40 inhibitor (e.g., CD40 antigen-binding molecule) is administered if the viral vector is still present in the subject. In some such methods, the CD40 inhibitor (e.g., CD40 antigen-binding molecule) is administered about 1 week prior to or within about 1 week prior to the nucleic acid construct.

In some such methods, the nucleic acid construct is administered simultaneously with the nuclease agent or the one or more nucleic acids encoding the nuclease agent. In some such methods, the nucleic acid construct is administered prior to or after the nuclease agent or the one or more nucleic acids encoding the nuclease agent.

In some such methods, the nucleic acid construct is in the nucleic acid vector, optionally wherein the nucleic acid vector is a viral vector. In some such methods, the viral vector is administered at a dose of about 3E11 vg/kg to about 5E13 vg/kg. In some such methods, the nucleic acid vector is an adeno-associated viral (AAV) vector. In some such methods, the nucleic acid construct is flanked by inverted terminal repeats (ITRs) on each end, optionally wherein the ITR on at least one end comprises, consists essentially of, or consists of SEQ ID NO: 112, and optionally wherein the ITR on each end comprises, consists essentially of, or consists of SEQ ID NO: 112, or optionally wherein the ITR on at least one end comprises, consists essentially of, or consists of SEQ ID NO: 110, and optionally wherein the ITR on each end comprises, consists essentially of, or consists of SEQ ID NO: 110. In some such methods, the AAV vector is a single-stranded AAV (ssAAV) vector. In some such methods, the AAV vector is a recombinant AAV8 (rAAV8) vector.

In some such methods, the polypeptide of interest is a factor IX protein. In some such methods, the factor IX protein coding sequence encodes a factor IX protein comprising SEQ ID NO: 63. In some such methods, the factor IX protein coding sequence comprises or consists of SEQ ID NO: 61, or wherein the factor IX protein coding sequence comprises or consists of SEQ ID NO: 60. In some such methods, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such methods, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 60 and the second factor IX protein coding sequence comprises SEQ ID NO: 61; or (ii) the first factor IX protein coding sequence comprises SEQ ID NO: 61 and the second factor IX protein coding sequence comprises SEQ ID NO: 60, wherein the nucleic acid construct does not comprise a promoter that drives the expression of the factor IX protein, and wherein the nucleic acid construct does not comprise homology arms. In some such methods, the nucleic acid construct comprises SEQ ID NO: 64 or 62 or the reverse complement thereof.

In some such methods, the polypeptide of interest is a multidomain therapeutic protein comprising a delivery domain fused to a lysosomal alpha-glucosidase. In some such methods, the lysosomal alpha-glucosidase comprises or consists of the sequence set forth in SEQ ID NO: 117. In some such methods, the lysosomal alpha-glucosidase coding sequence comprises or consist of the sequence set forth in SEQ ID NO: 127.

In some such methods, the delivery domain is a CD63-binding delivery domain. In some such methods, the CD63-binding delivery domain comprises an anti-CD63 antigen-binding protein. In some such methods, the CD63-binding delivery domain is a single-chain variable fragment (scFv). In some such methods, the scFv comprises or consists of the sequence set forth in SEQ ID NO: 119. In some such methods, the scFv coding sequence comprises or consists of the sequence set forth in SEQ ID NO: 129. In some such methods, the multidomain therapeutic protein comprises or consists of the sequence set forth in SEQ ID NO: 120. In some such methods, the coding sequence for the multidomain therapeutic protein comprises or consists of the sequence set forth in SEQ ID NO: 128, optionally wherein the nucleic acid construct comprises the sequence set forth in SEQ ID NO: 134 or 132. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 128, optionally wherein the nucleic acid construct comprises the sequence set forth in SEQ ID NO: 134 or 132, wherein the polyadenylation signal comprises a BGH polyadenylation signal and a unidirectional SV40 late polyadenylation signal, wherein the nucleic acid construct does not comprise a promoter that drives the expression of the multidomain therapeutic protein, and wherein the nucleic acid construct does not comprise a homology arm.

In some such methods, the delivery domain is a TfR-binding delivery domain. In some such methods, the TfR-binding delivery domain comprises an anti-TfR antigen-binding protein. In some such methods, the TfR-binding delivery domain comprises a single-chain variable fragment (scFv). In some such methods, the scFv comprises or consists of the sequence set forth in SEQ ID NO: 121. In some such methods, the scFv coding sequence comprises or consists of the sequence set forth in SEQ ID NO: 122. In some such methods, the multidomain therapeutic protein comprises or consists of the sequence set forth in SEQ ID NO: 126. In some such methods, the coding sequence for the multidomain therapeutic protein comprises or consists of the sequence set forth in SEQ ID NO: 125, optionally wherein the nucleic acid construct comprises the sequence set forth in SEQ ID NO: 133 or 130. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 125, optionally wherein the nucleic acid construct comprises the sequence set forth in SEQ ID NO: 133 or 130, wherein the polyadenylation signal comprises a BGH polyadenylation signal and a unidirectional SV40 late polyadenylation signal, wherein the nucleic acid construct does not comprise a promoter that drives the expression of the multidomain therapeutic protein, and wherein the nucleic acid construct does not comprise a homology arm.

In some such methods, the polypeptide of interest is a factor VIII protein. In some such methods, the polypeptide of interest is an antigen-binding protein, optionally wherein the antigen-binding protein is an antibody.

In some such methods, the target genomic locus is an albumin gene, optionally wherein the albumin gene is a human albumin gene. In some such methods, the nuclease target site is in intron 1 of the albumin gene.

In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the DNA-targeting segment comprises or consists of SEQ ID NO: 96. In some such methods, the guide RNA comprises SEQ ID NO: 100 or 104. In some such methods, the method comprises administering the guide RNA in the form of RNA. In some such methods, the guide RNA comprises at least one modification. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the method comprises administering the guide RNA in the form of RNA, the guide RNA comprises SEQ ID NO: 104, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein, optionally wherein the Cas protein is derived from a Streptococcus pyogenes Cas9 protein. In some such methods, the Cas protein comprises the sequence set forth in SEQ ID NO: 75. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is fully substituted with N1-methyl-pseudouridine. In some such methods, the mRNA encoding the Cas protein comprises the sequence set forth in SEQ ID NO: 65 or 66. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 65 or 66, and the mRNA encoding the Cas protein is fully substituted with N1-methyl-pseudouridine, comprises a 5′ cap, and comprises a poly(A) tail. In some such methods, the method comprises administering the guide RNA in the form of RNA, and the guide RNA comprises SEQ ID NO: 100 or 104, and wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 65 or 66. In some such methods, the method comprises administering the guide RNA in the form of RNA, the guide RNA comprises SEQ ID NO: 104, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 65 or 66, and the mRNA encoding the Cas protein is fully substituted with N1-methyl-pseudouridine, comprises a 5′ cap, and comprises a poly(A) tail.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or the neutral lipid is distearoylphosphatidylcholine or 1,2-distearoyl-sn-glycero-3-phosphocholine (DSPC), and/or wherein the helper lipid is cholesterol, and/or wherein the stealth lipid is 1,2-dimyristoyl-rac-glycero-3-methoxypolyethylene glycol-2000. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol % Lipid A, about 9 mol % DSPC, about 38 mol % cholesterol, and about 3 mol % PEG2k-DMG.

In some such methods, the cell is a liver cell or a hepatocyte, or the population of cells is a population of liver cells or hepatocytes. In some such methods, the subject is a human subject. In some such methods, the subject is a neonatal subject. In some such methods, the method further comprises determining whether the subject has immunity against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent prior to the administering, optionally wherein the determining comprises determining the presence of neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent. In some such methods, the nucleic acid construct is in an adeno-associated viral (AAV) vector, and wherein the subject does not have preexisting AAV immunity (e.g., at the time the CD40 inhibitor is administered). In some such methods, the subject does not have preexisting AAV immunity (i.e., naive or seronegative). In some such methods, the subject has preexisting AAV immunity.

In some such methods, the method further comprises administering to the subject a plasma cell depleting agent at a time when the subject has preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent when the plasma cell depleting agent is administered, optionally wherein the subject has preexisting AAV immunity when the plasma cell depleting agent is administered. In some such methods, the plasma cell depleting agent is capable of depleting long-lived plasma cells (LLPC). In some such methods, the plasma cell depleting agent is a B cell maturation antigen (BCMA) targeting agent. In some such methods, the BCMA targeting agent is a chimeric antigen receptor against BCMA or an anti-BCMA antibody or a functional fragment thereof. In some such methods, the anti-BCMA antibody or functional fragment thereof is conjugated to a cytotoxic agent. In some such methods, the anti-BCMA antibody is a multispecific antibody or a functional fragment thereof.

In some such methods, the multispecific anti-BCMA antibody or functional fragment thereof targets BCMA and CD3. In some such methods, the multispecific anti-BCMA antibody or functional fragment thereof is anti-BCMA×CD3 bispecific antibody or functional fragment thereof. In some such methods, the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B. In some such methods, the anti-BCMA×CD3 bispecific antibody or functional fragment thereof comprises a first antigen-binding domain that specifically binds to BCMA comprising three heavy chain CDRs (HCDR1, HCDR2 and HCDR3) contained within a heavy chain variable region (HCVR) comprising the amino acid sequence of SEQ ID NO: 287, and three light chain CDRs (LCDR1, LCDR2 and LCDR3) contained within a light chain variable region (LCVR) comprising the amino acid sequence of SEQ ID NO: 303. In some such methods, the first antigen-binding domain that specifically binds to BCMA comprises a HCDR1 comprising the amino acid sequence of SEQ ID NO: 289, a HCDR2 comprising the amino acid sequence of SEQ ID NO: 291, a HCDR3 comprising the amino acid sequence of SEQ ID NO: 293, a LCDR1 comprising the amino acid sequence of SEQ ID NO: 305, a LCDR2 comprising the amino acid sequence of SEQ ID NO: 307, and a LCDR3 comprising the amino acid sequence of SEQ ID NO: 309. In some such methods, the anti-BCMA×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence selected from the group consisting of SEQ ID NOS: 311 and 319, and three light chain CDRs (LCDR1, LCDR2 and LCDR3) contained within a light chain variable region (LCVR) comprising the amino acid sequence of SEQ ID NO: 303. In some such methods, the second antigen-binding domain that specifically binds to CD3 comprises a HCDR1 comprising the amino acid sequence of SEQ ID NO: 313 or 321, a HCDR2 comprising the amino acid sequence of SEQ ID NO: 315 or 323, a HCDR3 comprising the amino acid sequence of SEQ ID NO: 317 or 325, a LCDR1 comprising the amino acid sequence of SEQ ID NO: 305, a LCDR2 comprising the amino acid sequence of SEQ ID NO: 307, and a LCDR3 comprising the amino acid sequence of SEQ ID NO: 309. In some such methods, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 289, 291, and 293, respectively, and LCDR1, LCDR2, and LCDR3 comprising the amino acid sequences of SEQ ID NOS: 305, 307, and 309, respectively; and (b) a second antigen-binding domain that comprises HCDR1, HCDR2, and HCDR3 comprising the amino acid sequences of SEQ ID NOS: 313, 315, and 317, respectively, and LCDR1, LCDR2, and LCDR3 comprising the amino acid sequences of SEQ ID NOS: 305, 307, and 319, respectively. In some such methods, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 289, 291, and 293, respectively, and LCDR1, LCDR2, and LCDR3 comprising the amino acid sequences of SEQ ID NOS: 305, 307, and 309, respectively; and (b) a second antigen-binding domain that comprises HCDR1, HCDR2, and HCDR3 comprising the amino acid sequences of SEQ ID NOS: 321, 323, and 325, respectively, and LCDR1, LCDR2, and LCDR3 comprising the amino acid sequences of SEQ ID NOS: 305, 307, and 319, respectively. In some such methods, the anti-BCMA×CD3 bispecific antibody or functional fragment thereof comprises a human IgG heavy chain constant region. In some such methods, the human IgG heavy chain constant region is isotype IgG4 or IgG1. In some such methods, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR).

In some such methods, the method further comprises administering to the subject an effective amount of a B cell depleting agent (i.e., in combination with the plasma cell depleting agent) and/or an immunoglobulin depleting agent (i.e., in combination with the plasma cell depleting agent) at a time when the subject has preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent when the B cell depleting agent and/or the immunoglobulin depleting agent is administered, optionally wherein the subject has preexisting AAV immunity when the B cell depleting agent and/or the immunoglobulin depleting agent is administered. In some such methods, the method further comprises administering to the subject an effective amount of a B cell depleting agent (i.e., in combination with the plasma cell depleting agent) and an immunoglobulin depleting agent (i.e., in combination with the plasma cell depleting agent) at a time when the subject has preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent when the B cell depleting agent and the immunoglobulin depleting agent are administered, optionally wherein the subject has preexisting AAV immunity when the B cell depleting agent and the immunoglobulin depleting agent are administered. In some such methods, the B cell depleting agent is administered before, at the same time as, or after the plasma cell depleting agent. In some such methods, the immunoglobulin depleting agent is administered after the plasma cell depleting agent. In some such methods, the B cell depleting agent is administered prior to and after the nucleic acid construct and/or the nuclease agent or the one or more nucleic acids encoding the nuclease agent. In some such methods, the immunoglobulin depleting agent is administered prior to and after the nucleic acid construct and/or the nuclease agent or the one or more nucleic acids encoding the nuclease agent. In some such methods, the B cell depleting agent is administered prior to and after the nucleic acid construct. In some such methods, the immunoglobulin depleting agent is administered prior to and after the nucleic acid construct. In some such methods, the immunoglobulin depleting agent is administered after an initial dose of the plasma cell depleting agent, or wherein the immunoglobulin depleting agent is administered after an initial dose of the plasma cell depleting agent and after an initial dose of the B cell depleting agent. In some such methods, the B cell depleting agent is capable of depleting B cells and plasma cells that express low levels of BCMA. In some such methods, the B cell depleting agent is an agent that binds to a B cell surface molecule. In some such methods, the B cell depleting agent is selected from an anti-CD19 antibody, an anti-CD20 antibody, an anti-CD19 antibody and an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such methods, the B cell depleting agent is an anti-CD20 antibody or a functional fragment thereof, wherein the anti-CD20 antibody is a multispecific antibody or a functional fragment thereof. In some such methods, the multispecific anti-CD20 antibody or functional fragment thereof targets CD20 and CD3. In some such methods, the multispecific anti-CD20 antibody or functional fragment thereof is anti-CD20×CD3 bispecific antibody or functional fragment thereof. In some such methods, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 329, and three light chain CDRs (LCDR1, LCDR2 and LCDR3) contained within a light chain variable region (LCVR) comprising the amino acid sequence of SEQ ID NO: 330. In some such methods, the first antigen-binding domain that specifically binds to CD20 comprises a HCDR1 comprising the amino acid sequence of SEQ ID NO: 332, a HCDR2 comprising the amino acid sequence of SEQ ID NO: 333, a HCDR3 comprising the amino acid sequence of SEQ ID NO: 334, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337. In some such methods, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 331, and three light chain CDRs (LCDR1, LCDR2 and LCDR3) contained within a light chain variable region (LCVR) comprising the amino acid sequence of SEQ ID NO: 330. In some such methods, the second antigen-binding domain that specifically binds to CD3 comprises a HCDR1 comprising the amino acid sequence of SEQ ID NO: 338, a HCDR2 comprising the amino acid sequence of SEQ ID NO: 339, a HCDR3 comprising the amino acid sequence of SEQ ID NO: 340, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337. In some such methods, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 332, 333, and 334, respectively, and LCDR1, LCDR2, and LCDR3 comprising the amino acid sequences of SEQ ID NOS: 335, 336, and 337, respectively; and (b) a second antigen-binding domain that comprises HCDR1, HCDR2, and HCDR3 comprising the amino acid sequences of SEQ ID NOS: 338, 339, and 340, respectively, and LCDR1, LCDR2, and LCDR3 comprising the amino acid sequences of SEQ ID NOS: 335, 336, and 337, respectively. In some such methods, the anti-CD20×CD3 bispecific antibody or functional fragment thereof comprises a human IgG heavy chain constant region. In some such methods, the human IgG heavy chain constant region is isotype IgG4 or IgG1. In some such methods, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such methods, the B cell depleting agent is an agent targeting a B cell survival factor. In some such methods, the B cell depleting agent is a BLyS/BAFF inhibitor, an APRIL inhibitor, a BLyS receptor 3/BAFF receptor inhibitor, or any combination thereof.

In some such methods, the immunoglobulin depleting agent is capable of accelerating IgG clearance. In some such methods, the immunoglobulin depleting agent is a neonatal Fc receptor (FcRn) blocker. In some such methods, the FcRn blocker is selected from Efgartigimod (ARGX-113), Rozanolixizumab (UCB7665), Batoclimab (RVT-1401), IMVT-1402, Nipocalimab (M281), Orilanolimab (SYNT001), and any combinations thereof.

In some such methods, the immunoglobulin depleting agent is an immunoglobulin degrading enzyme. In some such methods, the CD40 inhibitor is resistant to the immunoglobulin degrading enzyme. In some such methods, the immunoglobulin degrading enzyme is selected from Imlifidase/IdeS/Fabricator, IdeZ, IdeXork, IceMG, CYR-212, CYR-241, s-1117, HNSA-5487, and IdeE/KJ103. In some such methods, the immunoglobulin degrading enzyme is Imlifidase/IdeS/Fabricator. In some such methods, the CD40 inhibitor is administered before administration of the immunoglobulin depleting agent to the subject, and the immunoglobulin depleting agent is administered before the administration of the nucleic acid construct and/or the nuclease agent or the one or more nucleic acids encoding the nuclease agent to the subject. In some such methods, the subject does not have preexisting immunity against the immunoglobulin degrading enzyme. In some such methods, the subject has preexisting immunity against the immunoglobulin degrading enzyme.

In some such methods, the method further comprises plasmapheresis, therapeutic plasma exchange, or immunoadsorption.

In some such methods, the plasma cell depleting agent is administered simultaneously with the nucleic acid construct and/or the nuclease agent or the one or more nucleic acids encoding the nuclease agent. In some such methods, the plasma cell depleting agent is administered prior to the nucleic acid construct and/or the nuclease agent or the one or more nucleic acids encoding the nuclease agent. In some such methods, the plasma cell depleting agent is administered prior to and after the nucleic acid construct and/or the nuclease agent or the one or more nucleic acids encoding the nuclease agent. In some such methods, the plasma cell depleting agent is administered within about 6 months after the nucleic acid construct and/or the nuclease agent or the one or more nucleic acids encoding the nuclease agent. In some such methods, the plasma cell depleting agent is administered about 1 week prior to or within about 1 week prior to the nucleic acid construct and/or the nuclease agent or the one or more nucleic acids encoding the nuclease agent.

In some such methods, the plasma cell depleting agent is administered simultaneously with the nucleic acid construct. In some such methods, the plasma cell depleting agent is administered prior to the nucleic acid construct. In some such methods, the plasma cell depleting agent is administered prior to and after the nucleic acid construct. In some such methods, the plasma cell depleting agent is administered within about 6 months after the nucleic acid construct, optionally wherein the nucleic acid construct is in a viral vector, and the plasma cell depleting agent is administered if the viral vector is still present in the subject. In some such methods, the plasma cell depleting agent is administered about 1 week prior to or within about 1 week prior to the nucleic acid construct.

In some such methods, the nucleic acid construct is administered simultaneously with the nuclease agent or the one or more nucleic acids encoding the nuclease agent. In some such methods, the nucleic acid construct is administered prior to or after the nuclease agent or the one or more nucleic acids encoding the nuclease agent.

In some such methods, the subject has preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, wherein the plasma cell depleting agent and the CD40 inhibitor are both administered prior to the nucleic acid construct and the nuclease agent or the one or more nucleic acids encoding the nuclease agent, and the plasma cell depleting agent is administered prior to the CD40 inhibitor. In some such methods, the subject has preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, wherein the plasma cell depleting agent and the CD40 inhibitor are both administered prior to the nucleic acid construct and the nuclease agent or the one or more nucleic acids encoding the nuclease agent, and the plasma cell depleting agent and the CD40 inhibitor are administered simultaneously, optionally wherein the subject has ongoing B cell responses to the nucleic acid construct, the polypeptide of interest, the nuclease agent, the one or more nucleic acids encoding the nuclease agent, or the delivery vehicle when the plasma cell depleting agent and the CD40 inhibitor are administered. In some such methods, the subject does not have preexisting immunity against the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent before administration of the nucleic acid construct and the nuclease agent or the one or more nucleic acids encoding the nuclease agent, the CD40 inhibitor is administered prior to the nucleic acid construct and the nuclease agent or the one or more nucleic acids encoding the nuclease agent, the subject develops immunity against the polypeptide of interest, the nuclease agent, the one or more nucleic acids encoding the nuclease agent, or the delivery vehicle from the administration of the nucleic acid construct and the nuclease agent or the one or more nucleic acids encoding the nuclease agent, and the plasma cell depleting agent is administered after the administration of the nucleic acid construct and the nuclease agent or the one or more nucleic acids encoding the nuclease agent but prior to a second dose of the nucleic acid construct, a second dose of the nuclease agent or the one or more nucleic acids encoding the nuclease agent, a second nucleic acid construct, and/or a second nuclease agent or one or more nucleic acids encoding the second nuclease agent. Optionally, the plasma cell depleting agent is administered in combination with a B cell depleting agent and/or an immunoglobulin depleting agent (i.e., at a time when the subject has preexisting immunity against the nucleic acid construct, the polypeptide of interest, the nuclease agent, the one or more nucleic acids encoding the nuclease agent, or the delivery vehicle).

In some such methods, the nucleic acid construct is in an AAV vector, the subject has preexisting immunity against the AAV vector, and the plasma cell depleting agent and the CD40 inhibitor are both administered prior to the nucleic acid construct, wherein the plasma cell depleting agent is administered prior to the CD40 inhibitor. Optionally, the plasma cell depleting agent is administered in combination with a B cell depleting agent and/or an immunoglobulin depleting agent (i.e., when the subject has preexisting immunity against the AAV vector). In some such methods, the nucleic acid construct is in an AAV vector, the subject has preexisting immunity against the AAV vector, and the plasma cell depleting agent and the CD40 inhibitor are both administered prior to the nucleic acid construct, wherein the plasma cell depleting agent and the CD40 inhibitor are administered simultaneously, optionally wherein the subject has ongoing B cell responses to the AAV vector when the plasma cell depleting agent and the CD40 inhibitor are administered. Optionally, the plasma cell depleting agent is administered in combination with a B cell depleting agent and/or an immunoglobulin depleting agent (i.e., when the subject has preexisting immunity against the AAV vector). In some such methods, the nucleic acid construct is in an AAV vector, the subject does not have preexisting immunity against the AAV vector before administration of a first dose of the nucleic acid construct, the CD40 inhibitor is administered prior to the first dose of the nucleic acid construct, the subject develops immunity against the AAV vector from the first dose of the nucleic acid construct, and the plasma cell depleting agent is administered after the first dose of the nucleic acid construct but prior to a second dose of the nucleic acid construct. Optionally, the plasma cell depleting agent is administered in combination with a B cell depleting agent and/or an immunoglobulin depleting agent (i.e., when the subject has preexisting immunity against the AAV vector). In some such methods, the nucleic acid construct is in an AAV vector, the subject does not have preexisting immunity against the AAV vector before administration of the nucleic acid construct, the CD40 inhibitor is administered prior to the nucleic acid construct, the subject develops immunity against the AAV vector from administration of the nucleic acid construct, and the plasma cell depleting agent is administered after the nucleic acid construct but prior to administration of a second nucleic acid construct (e.g., in an AAV vector, e.g., an AAV vector of the same serotype as the first nucleic acid construct). Optionally, the plasma cell depleting agent is administered in combination with a B cell depleting agent and/or an immunoglobulin depleting agent (i.e., when the subject has preexisting immunity against the AAV vector).

In another aspect, provided are compositions or combinations comprising a CD40 inhibitor (e.g., CD40 antigen-binding molecule) in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

In some such compositions or combinations, the CD40 inhibitor is a CD40 antigen-binding molecule. In some such compositions or combinations, the CD40 antigen-binding molecule is a monospecific antigen-binding molecule. In some such compositions or combinations, the CD40 antigen-binding molecule is a bispecific antigen-binding molecule comprising: (a) a first antigen-binding domain (D1) that binds a first epitope of human CD40; and (b) a second antigen-binding domain (D2) that binds a second epitope of human CD40. In some such compositions or combinations, the bispecific antigen-binding molecule: (i) binds human CD40 with a K_{D} of less than 25 nM as measured by surface plasmon resonance at 25° C.; (ii) binds human CD40 with a K_{D} of less than 70 nM as measured by surface plasmon resonance at 37° C.; (iii) binds human CD40 with a dissociative half-life (t½) of greater than 75 minutes as measured by surface plasmon resonance at 25° C.; (iv) binds a human CD40-expressing cell with an EC₅₀ value of about 10 nM or less; (v) inhibits binding of human CD40 dimer to CD40L; (vi) inhibits CD40 ligand (CD40L)-induced activation; and/or (vii) does not significantly agonize CD40 in the absence of CD40L. In some such compositions or combinations, the bispecific antigen-binding molecule: (i) binds human CD40 with a K_{D} of less than 25 nM as measured by surface plasmon resonance at 25° C.; (ii) binds human CD40 with a K_{D} of less than 70 nM as measured by surface plasmon resonance at 37° C.; (iii) binds human CD40 with a dissociative half-life (t½) of greater than 75 minutes as measured by surface plasmon resonance at 25° C.; (iv) binds a human CD40-expressing cell with an EC₅₀ value of about 10 nM or less; (v) inhibits binding of human CD40 monomer to CD40L; (vi) inhibits CD40 ligand (CD40L)-induced activation; and/or (vii) does not significantly agonize CD40 in the absence of CD40L. In some such compositions or combinations, the bispecific antigen-binding molecule inhibits CD40L-induced activation. In some such compositions or combinations, the bispecific antigen-binding molecule inhibits CD40L-induced activation and does not significantly agonize CD40 in the absence of CD40L. In some such compositions or combinations, the D1 domain and the D2 domain each comprise a heavy chain immunoglobulin variable region comprising a set of three heavy chain complementarity determining region sequences HCDR1, HCDR2, and HCDR3 independently selected from the group consisting of: (a) an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; (b) an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28; and (c) an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38. In some such compositions or combinations, the D1 domain and the D2 domain each comprise a light chain immunoglobulin variable region comprising a set of three light chain complementarity determining region sequences LCDR1, LCDR2, and LCDR3, wherein the LCDR1 comprises the amino acid sequence of SEQ ID NO: 12, the LCDR2 comprises the amino acid sequence AAS (SEQ ID NO: 14), and the LCDR3 comprises the amino acid sequence of SEQ ID NO: 16.

In some such compositions or combinations, the D1 domain comprises: (a) an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; or (b) an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 domain comprises an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 domain comprises a heavy chain variable region (HCVR) comprising the amino acid sequence of SEQ ID NO: 2. In some such compositions or combinations, the D1 domain comprises an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 domain comprises an HCVR comprising the amino acid sequence of SEQ ID NO: 22.

In some such compositions or combinations, the D1 domain comprises a light chain variable region (LCVR) comprising the amino acid sequence of SEQ ID NO: 10. In some such compositions or combinations, the D2 domain comprises an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D2 domain comprises an HCVR comprising the amino acid sequence of SEQ ID NO: 32. In some such compositions or combinations, the D2 domain comprises an LCVR comprising the amino acid sequence of SEQ ID NO: 10.

In some such compositions or combinations, the bispecific antigen-binding molecule comprises: a D1 comprising an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 comprises an HCVR comprising the amino acid sequence of SEQ ID NO: 2 and an LCVR comprising the amino acid sequence of SEQ ID NO: 10, and the D2 comprises an HCVR comprising the amino acid sequence of SEQ ID NO: 32 and an LCVR comprising the amino acid sequence of SEQ ID NO: 10. In some such compositions or combinations, the bispecific antigen-binding molecule comprises: a D1 comprising an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 comprises an HCVR comprising the amino acid sequence of SEQ ID NO: 22 and an LCVR comprising the amino acid sequence of SEQ ID NO: 10, and the D2 comprises an HCVR comprising the amino acid sequence of SEQ ID NO: 32 and an LCVR comprising the amino acid sequence of SEQ ID NO: 10.

In some such compositions or combinations, the D1 domain comprises an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D1 domain comprises an HCVR comprising the amino acid sequence of SEQ ID NO: 32. In some such compositions or combinations, the D1 domain comprises an LCVR comprising the amino acid sequence of SEQ ID NO: 10.

In some such compositions or combinations, the D2 domain comprises an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some such compositions or combinations, the D2 domain comprises an HCVR comprising the amino acid sequence of SEQ ID NO: 2. In some such compositions or combinations, the D2 domain comprises an LCVR comprising the amino acid sequence of SEQ ID NO: 10.

In some such compositions or combinations, the bispecific antigen-binding molecule comprises: a D1 comprising an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; and a D2 comprising an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some such compositions or combinations, the D1 comprises an HCVR comprising the amino acid sequence of SEQ ID NO: 32 and an LCVR comprising the amino acid sequence of SEQ ID NO: 10, and the D2 comprises an HCVR comprising the amino acid sequence of SEQ ID NO: 2 and an LCVR comprising the amino acid sequence of SEQ ID NO: 10.

In some such compositions or combinations, the bispecific antigen-binding molecule is a bispecific antibody. In some such compositions or combinations, the bispecific antibody comprises a human IgG heavy chain constant region. In some such compositions or combinations, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such compositions or combinations, the human IgG heavy chain constant region is isotype IgG4 or IgG1. In some such compositions or combinations, the human IgG heavy chain constant region comprises one or more modifications in a hinge region. In some such compositions or combinations, the human IgG heavy chain constant region comprises one or more modifications that reduce binding to an Fc receptor (e.g., one or more modifications in a hinge region and/or CH region). In some such compositions or combinations, the D1 comprises: a heavy chain comprising the amino acid sequence of SEQ ID NO: 42 and a light chain comprising the amino acid sequence of SEQ ID NO: 20; or a heavy chain comprising the amino acid sequence of SEQ ID NO: 46 and a light chain comprising the amino acid sequence of SEQ ID NO: 20; or a heavy chain comprising the amino acid sequence of SEQ ID NO: 48 and a light chain comprising the amino acid sequence of SEQ ID NO: 20; or a heavy chain comprising the amino acid sequence of SEQ ID NO: 52 and a light chain comprising the amino acid sequence of SEQ ID NO: 20. In some such compositions or combinations, the D1 comprises: a heavy chain comprising the amino acid sequence of SEQ ID NO: 283 and a light chain comprising the amino acid sequence of SEQ ID NO: 20. In some such compositions or combinations, the D2 comprises: a heavy chain comprising the amino acid sequence of SEQ ID NO: 44 and a light chain comprising the amino acid sequence of SEQ ID NO: 20; or a heavy chain comprising the amino acid sequence of SEQ ID NO: 50 and a light chain comprising the amino acid sequence of SEQ ID NO: 20. In some such compositions or combinations, the D2 comprises: a heavy chain comprising the amino acid sequence of SEQ ID NO: 285 and a light chain comprising the amino acid sequence of SEQ ID NO: 20.

In some such compositions or combinations, the bispecific antigen-binding molecule is a bispecific antibody comprising (i) a D1 comprising a heavy chain comprising the amino acid sequence of SEQ ID NO: 42 and a light chain comprising the amino acid sequence of SEQ ID NO: 20; and (ii) a D2 comprising a heavy chain comprising the amino acid sequence of SEQ ID NO: 44 and a light chain comprising the amino acid sequence of SEQ ID NO: 20. In some such compositions or combinations, the bispecific antigen-binding molecule is a bispecific antibody comprising (i) a D1 comprising a heavy chain comprising the amino acid sequence of SEQ ID NO: 46 and a light chain comprising the amino acid sequence of SEQ ID NO: 20; and (ii) a D2 comprising a heavy chain comprising the amino acid sequence of SEQ ID NO: 44 and a light chain comprising the amino acid sequence of SEQ ID NO: 20. In some such compositions or combinations, the bispecific antigen-binding molecule is a bispecific antibody comprising (i) a D1 comprising a heavy chain comprising the amino acid sequence of SEQ ID NO: 48 and a light chain comprising the amino acid sequence of SEQ ID NO: 20; and (ii) a D2 comprising a heavy chain comprising the amino acid sequence of SEQ ID NO: 50 and a light chain comprising the amino acid sequence of SEQ ID NO: 20. In some such compositions or combinations, the bispecific antigen-binding molecule is a bispecific antibody comprising (i) a D1 comprising a heavy chain comprising the amino acid sequence of SEQ ID NO: 52 and a light chain comprising the amino acid sequence of SEQ ID NO: 20; and (ii) a D2 comprising a heavy chain comprising the amino acid sequence of SEQ ID NO: 50 and a light chain comprising the amino acid sequence of SEQ ID NO: 20.

In some such compositions or combinations, the D1 comprises a heavy chain comprising the amino acid sequence of SEQ ID NO: 283 and a light chain comprising the amino acid sequence of SEQ ID NO: 20. In some such compositions or combinations, the D2 comprises a heavy chain comprising the amino acid sequence of SEQ ID NO: 285 and a light chain comprising the amino acid sequence of SEQ ID NO: 20. In some such compositions or combinations, the bispecific antigen-binding molecule is a bispecific antibody comprising (i) a D1 comprising a heavy chain comprising the amino acid sequence of SEQ ID NO: 283 and a light chain comprising the amino acid sequence of SEQ ID NO: 20; and (ii) a D2 comprising a heavy chain comprising the amino acid sequence of SEQ ID NO: 285 and a light chain comprising the amino acid sequence of SEQ ID NO: 20.

In some such compositions or combinations, the bispecific antigen-binding molecule is a bispecific antibody having the amino acid sequences of REGN16334. In some such compositions or combinations, the bispecific antigen-binding molecule is a bispecific antibody having the amino acid sequences of REGN16335. In some such compositions or combinations, the bispecific antigen-binding molecule is a bispecific antibody having the amino acid sequences of REGN16431. In some such compositions or combinations, the bispecific antigen-binding molecule is a bispecific antibody having the amino acid sequences of REGN16432. In some such compositions or combinations, the bispecific antigen-binding molecule is a bispecific antibody having the amino acid sequences of REGN20484.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within an HCVR/LCVR amino acid sequence pair selected from the group consisting of SEQ ID NOS: 2/10, 22/10, 32/10, 183/184, 141/142, 151/268, 231/232, 160/161, 170/171, 173/174, 188/189, 199/200, 183/212, 183/215, 183/217, 211/184, 211/212, 211/215, 211/217, 213/184, 213/212, 213/215, 213/217, 214/184, 214/212, 214/215, 214/217, 216/184, 216/212, 216/215, 216/217, 224/225, 244/245, 251/252, 259/260, 264/265, and 259/265; (II) the HCVR and LCVR amino acid sequences contained within an HCVR/LCVR amino acid sequence pair selected from the group consisting of SEQ ID NOS: 2/10, 22/10, 32/10, 183/184, 141/142, 151/268, 231/232, 160/161, 170/171, 173/174, 188/189, 199/200, 183/212, 183/215, 183/217, 211/184, 211/212, 211/215, 211/217, 213/184, 213/212, 213/215, 213/217, 214/184, 214/212, 214/215, 214/217, 216/184, 216/212, 216/215, 216/217, 224/225, 244/245, 251/252, 259/260, 264/265, and 259/265; or (III) the heavy chain and light chain amino acid sequences contained within a heavy chain/light chain amino acid sequence pair selected from the group consisting of SEQ ID NOS: 18/20, 30/20, 40/20, 185/186, 143/144, 152/153, 236/237, 162/163, 172/144, 175/176, 190/192, 191/192, 201/202, 218/153, 229/230, 233/237, 234/237, 235/237, 246/250, 247/250, 248/250, 249/250, 261/262, 266/267, and 261/267.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within an HCVR/LCVR amino acid sequence pair selected from the group consisting of SEQ ID NOS: 2/10, 22/10, and 32/10; (II) (a) an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; or (b) an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; or (c) an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence of SEQ ID NO: 14, and an LCDR3 comprising the amino acid sequence of SEQ ID NO: 16; (III) (a) an HCVR comprising or consisting of the amino acid sequence of SEQ ID NO: 2 and an LCVR comprising or consisting of the amino acid sequence of SEQ ID NO: 10; (b) an HCVR comprising or consisting of the amino acid sequence of SEQ ID NO: 22 and an LCVR comprising or consisting of the amino acid sequence of SEQ ID NO: 10; (c) an HCVR comprising or consisting of the amino acid sequence of SEQ ID NO: 32 and an LCVR comprising or consisting of the amino acid sequence of SEQ ID NO: 10; or (IV) (a) a heavy chain comprising or consisting of the amino acid sequence of SEQ ID NO: 18 and a light chain comprising or consisting of the amino acid sequence of SEQ ID NO: 20; (b) a heavy chain comprising or consisting of the amino acid sequence of SEQ ID NO: 30 and a light chain comprising or consisting of the amino acid sequence of SEQ ID NO: 20; or (c) a heavy chain comprising or consisting of the amino acid sequence of SEQ ID NO: 40 and a light chain comprising or consisting of the amino acid sequence of SEQ ID NO: 20.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 183/184; (II) an HCDR1 comprising the amino acid sequence of SEQ ID NO: 177, an HCDR2 comprising the amino acid sequence of SEQ ID NO: 178, an HCDR3 comprising the amino acid sequence of SEQ ID NO: 179, an LCDR1 comprising the amino acid sequence of SEQ ID NO: 180, an LCDR2 comprising the amino acid sequence of SEQ ID NO: 181, and an LCDR3 comprising the amino acid sequence of SEQ ID NO: 182; (III) an HCVR comprising or consisting of the amino acid sequence of SEQ ID NO: 183 and an LCVR comprising or consisting of the amino acid sequence of SEQ ID NO: 184; or (IV) a heavy chain comprising or consisting of the amino acid sequence of SEQ ID NO: 185 and a light chain comprising or consisting of the amino acid sequence of SEQ ID NO: 186.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 141/142; (II) an HCDR1 comprising the amino acid sequence of SEQ ID NO: 135, an HCDR2 comprising the amino acid sequence of SEQ ID NO: 136, an HCDR3 comprising the amino acid sequence of SEQ ID NO: 137, an LCDR1 comprising the amino acid sequence of SEQ ID NO: 138, an LCDR2 comprising the amino acid sequence of SEQ ID NO: 139, and an LCDR3 comprising the amino acid sequence of SEQ ID NO: 140; (III) an HCVR comprising or consisting of the amino acid sequence of SEQ ID NO: 141 and an LCVR comprising or consisting of the amino acid sequence of SEQ ID NO: 142; or (IV) a heavy chain comprising or consisting of the amino acid sequence of SEQ ID NO: 143 and a light chain comprising or consisting of the amino acid sequence of SEQ ID NO: 144.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 151/268; (II) an HCDR1 comprising the amino acid sequence of SEQ ID NO: 145, an HCDR2 comprising the amino acid sequence of SEQ ID NO: 146, an HCDR3 comprising the amino acid sequence of SEQ ID NO: 147, an LCDR1 comprising the amino acid sequence of SEQ ID NO: 148, an LCDR2 comprising the amino acid sequence of SEQ ID NO: 149, and an LCDR3 comprising the amino acid sequence of SEQ ID NO: 150; (III) an HCVR comprising or consisting of the amino acid sequence of SEQ ID NO: 151 and an LCVR comprising or consisting of the amino acid sequence of SEQ ID NO: 268; or (IV) a heavy chain comprising or consisting of the amino acid sequence of SEQ ID NO: 152 and a light chain comprising or consisting of the amino acid sequence of SEQ ID NO: 153.

In some such compositions or combinations, the CD40 antigen binding molecule is an anti-CD40 antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 231/232; (II) an HCDR1 comprising the amino acid sequence of SEQ ID NO: 269, an HCDR2 comprising the amino acid sequence of SEQ ID NO: 220, an HCDR3 comprising the amino acid sequence of SEQ ID NO: 221, an LCDR1 comprising the amino acid sequence of SEQ ID NO: 222, an LCDR2 comprising the amino acid sequence of SEQ ID NO: 223, and an LCDR3 comprising the amino acid sequence of SEQ ID NO: 150; (III) an HCVR comprising or consisting of the amino acid sequence of SEQ ID NO: 231 and an LCVR comprising or consisting of the amino acid sequence of SEQ ID NO: 232; or (IV) a heavy chain comprising or consisting of the amino acid sequence of SEQ ID NO: 236 and a light chain comprising or consisting of the amino acid sequence of SEQ ID NO: 237.

In some such compositions or combinations, the CD40 inhibitor is a CD40L binding protein. In some such compositions or combinations, (I) the CD40L binding protein comprises two Tn3 proteins fused to human serum albumin; or (II) the CD40L binding protein comprises the sequence set forth in SEQ ID NO: 280. In some such compositions or combinations, the CD40L binding protein is an anti-CD40L antibody or antigen-binding fragment thereof comprising: (I) the HCDR1-HCDR2-HCDR3-LCDR1-LCDR2-LCDR3 amino acid sequences set contained within the HCVR/LCVR amino acid sequence pair set forth in SEQ ID NOS: 276/277; (II) an HCDR1 comprising the amino acid sequence of SEQ ID NO: 270, an HCDR2 comprising the amino acid sequence of SEQ ID NO: 271, an HCDR3 comprising the amino acid sequence of SEQ ID NO: 272, an LCDR1 comprising the amino acid sequence of SEQ ID NO: 273, an LCDR2 comprising the amino acid sequence of SEQ ID NO: 274, and an LCDR3 comprising the amino acid sequence of SEQ ID NO: 275; (III) an HCVR comprising or consisting of the amino acid sequence of SEQ ID NO: 276 and an LCVR comprising or consisting of the amino acid sequence of SEQ ID NO: 277; or (IV) a heavy chain comprising or consisting of the amino acid sequence of SEQ ID NO: 278 and a light chain comprising or consisting of the amino acid sequence of SEQ ID NO: 279.

In some such compositions or combinations, the nucleic acid construct is in the nucleic acid vector. In some such compositions or combinations, the nucleic acid vector is a viral vector. In some such compositions or combinations, the nucleic acid vector is an adeno-associated viral (AAV) vector. In some such compositions or combinations, the nucleic acid construct is flanked by inverted terminal repeats (ITRs) on each end, optionally wherein the ITR on at least one end comprises, consists essentially of, or consists of SEQ ID NO: 112, and optionally wherein the ITR on each end comprises, consists essentially of, or consists of SEQ ID NO: 112, or optionally wherein the ITR on at least one end comprises, consists essentially of, or consists of SEQ ID NO: 110, and optionally wherein the ITR on each end comprises, consists essentially of, or consists of SEQ ID NO: 110. In some such compositions or combinations, the AAV vector is a single-stranded AAV (ssAAV) vector. In some such compositions or combinations, the AAV vector is a recombinant AAV8 (rAAV8) vector.

In some such compositions or combinations, the polypeptide of interest is a factor IX protein. In some such compositions or combinations, the factor IX protein coding sequence encodes a factor IX protein comprising SEQ ID NO: 63. In some such compositions or combinations, the factor IX protein coding sequence comprises or consists of SEQ ID NO: 61, or wherein the factor IX protein coding sequence comprises or consists of SEQ ID NO: 60. In some such compositions or combinations, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such compositions or combinations, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 60 and the second factor IX protein coding sequence comprises SEQ ID NO: 61; or (ii) the first factor IX protein coding sequence comprises SEQ ID NO: 61 and the second factor IX protein coding sequence comprises SEQ ID NO: 60, wherein the nucleic acid construct does not comprise a promoter that drives the expression of the factor IX protein, and wherein the nucleic acid construct does not comprise homology arms. In some such compositions or combinations, the nucleic acid construct comprises SEQ ID NO: 64 or 62 or the reverse complement thereof.

In some such compositions or combinations, the polypeptide of interest is a multidomain therapeutic protein comprising a delivery domain fused to a lysosomal alpha-glucosidase. In some such compositions or combinations, the lysosomal alpha-glucosidase comprises or consists of the sequence set forth in SEQ ID NO: 117. In some such compositions or combinations, the lysosomal alpha-glucosidase coding sequence comprises or consist of the sequence set forth in SEQ ID NO: 127.

In some such compositions or combinations, the delivery domain is a CD63-binding delivery domain. In some such compositions or combinations, the CD63-binding delivery domain comprises an anti-CD63 antigen-binding protein. In some such compositions or combinations, the CD63-binding delivery domain is a single-chain variable fragment (scFv). In some such compositions or combinations, the scFv comprises or consists of the sequence set forth in SEQ ID NO: 119. In some such compositions or combinations, the scFv coding sequence comprises or consists of the sequence set forth in SEQ ID NO: 129. In some such compositions or combinations, the multidomain therapeutic protein comprises or consists of the sequence set forth in SEQ ID NO: 120. In some such compositions or combinations, the coding sequence for the multidomain therapeutic protein comprises or consists of the sequence set forth in SEQ ID NO: 128, optionally wherein the nucleic acid construct comprises the sequence set forth in SEQ ID NO: 134 or 132. In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 128, optionally wherein the nucleic acid construct comprises the sequence set forth in SEQ ID NO: 134 or 132, wherein the polyadenylation signal comprises a BGH polyadenylation signal and a unidirectional SV40 late polyadenylation signal, wherein the nucleic acid construct does not comprise a promoter that drives the expression of the multidomain therapeutic protein, and wherein the nucleic acid construct does not comprise a homology arm.

In some such compositions or combinations, the delivery domain is a TfR-binding delivery domain. In some such compositions or combinations, the TfR-binding delivery domain comprises an anti-TfR antigen-binding protein. In some such compositions or combinations, the TfR-binding delivery domain comprises a single-chain variable fragment (scFv). In some such compositions or combinations, the scFv comprises or consists of the sequence set forth in SEQ ID NO: 121. In some such compositions or combinations, the scFv coding sequence comprises or consists of the sequence set forth in SEQ ID NO: 122. In some such compositions or combinations, the multidomain therapeutic protein comprises or consists of the sequence set forth in SEQ ID NO: 126. In some such compositions or combinations, the coding sequence for the multidomain therapeutic protein comprises or consists of the sequence set forth in SEQ ID NO: 125, optionally wherein the nucleic acid construct comprises the sequence set forth in SEQ ID NO: 133 or 130. In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 125, optionally wherein the nucleic acid construct comprises the sequence set forth in SEQ ID NO: 133 or 130, wherein the polyadenylation signal comprises a BGH polyadenylation signal and a unidirectional SV40 late polyadenylation signal, wherein the nucleic acid construct does not comprise a promoter that drives the expression of the multidomain therapeutic protein, and wherein the nucleic acid construct does not comprise a homology arm.

In some such compositions or combinations, the polypeptide of interest is a factor VIII protein. In some such compositions or combinations, the polypeptide of interest is an antigen-binding protein, optionally wherein the antigen-binding protein is an antibody.

In some such compositions or combinations, the target genomic locus is an albumin gene, optionally wherein the albumin gene is a human albumin gene. In some such compositions or combinations, the nuclease target site is in intron 1 of the albumin gene.

In some such compositions or combinations, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions or combinations, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions or combinations, the DNA-targeting segment comprises or consists of SEQ ID NO: 96. In some such compositions or combinations, the guide RNA comprises SEQ ID NO: 100 or 104. In some such compositions or combinations, the composition or combination comprises the guide RNA in the form of RNA. In some such compositions or combinations, the guide RNA comprises at least one modification. In some such compositions or combinations, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions or combinations, the composition or combination comprises the guide RNA in the form of RNA, the guide RNA comprises SEQ ID NO: 104, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions or combinations, the Cas protein is a Cas9 protein, optionally wherein the Cas protein is derived from a Streptococcus pyogenes Cas9 protein. In some such compositions or combinations, the Cas protein comprises the sequence set forth in SEQ ID NO: 75. In some such compositions or combinations, the composition or combination comprises the nucleic acid encoding the Cas protein, wherein the nucleic acid comprises an mRNA encoding the Cas protein. In some such compositions or combinations, the mRNA encoding the Cas protein comprises at least one modification. In some such compositions or combinations, the mRNA encoding the Cas protein is fully substituted with N1-methyl-pseudouridine. In some such compositions or combinations, the mRNA encoding the Cas protein comprises the sequence set forth in SEQ ID NO: 65 or 66. In some such compositions or combinations, the composition or combination comprises the nucleic acid encoding the Cas protein, wherein the nucleic acid comprises an mRNA encoding the Cas protein, the mRNA encoding the Cas protein comprises the sequence set forth in SEQ ID NO: 65 or 66, and the mRNA encoding the Cas protein is fully substituted with N1-methyl-pseudouridine, comprises a 5′ cap, and comprises a poly(A) tail. In some such compositions or combinations, the composition or combination comprises the guide RNA in the form of RNA, and the guide RNA comprises SEQ ID NO: 100 or 104, and wherein the composition or combination comprises administering the nucleic acid encoding the Cas protein, wherein the nucleic acid comprises an mRNA encoding the Cas protein, and the mRNA encoding the Cas protein comprises the sequence set forth in SEQ ID NO: 65 or 66. In some such compositions or combinations, the composition or combination comprises the guide RNA in the form of RNA, the guide RNA comprises SEQ ID NO: 104, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition or combination comprises the nucleic acid encoding the Cas protein, wherein the nucleic acid comprises an mRNA encoding the Cas protein, the mRNA encoding the Cas protein comprises the sequence set forth in SEQ ID NO: 65 or 66, and the mRNA encoding the Cas protein is fully substituted with N1-methyl-pseudouridine, comprises a 5′ cap, and comprises a poly(A) tail.

In some such compositions or combinations, the Cas protein or the nucleic acid encoding the Cas protein and the guide RNA or the one or more DNAs encoding the guide RNA are associated with a lipid nanoparticle. In some such compositions or combinations, the lipid nanoparticle comprises a cationic lipid, a neutral lipid, a helper lipid, and a stealth lipid. In some such compositions or combinations, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or wherein the neutral lipid is distearoylphosphatidylcholine or 1,2-distearoyl-sn-glycero-3-phosphocholine (DSPC), and/or wherein the helper lipid is cholesterol, and/or wherein the stealth lipid is 1,2-dimyristoyl-rac-glycero-3-methoxypolyethylene glycol-2000. In some such compositions or combinations, the cationic lipid is Lipid A, the neutral lipid is DSPC, the helper lipid is cholesterol, and the stealth lipid is PEG2k-DMG. In some such compositions or combinations, the lipid nanoparticle comprises four lipids at the following molar ratios: about 50 mol % Lipid A, about 9 mol % DSPC, about 38 mol % cholesterol, and about 3 mol % PEG2k-DMG.

In some such compositions or combinations, the CD40 inhibitor is further in combination with a plasma cell depleting agent. In some such compositions or combinations, the plasma cell depleting agent is capable of depleting long-lived plasma cells (LLPC). In some such compositions or combinations, the plasma cell depleting agent is a B cell maturation antigen (BCMA) targeting agent. In some such compositions or combinations, the BCMA targeting agent is a chimeric antigen receptor against BCMA or an anti-BCMA antibody or a functional fragment thereof. In some such compositions or combinations, the anti-BCMA antibody or functional fragment thereof is conjugated to a cytotoxic agent. In some such compositions or combinations, the anti-BCMA antibody is a multispecific antibody or a functional fragment thereof.

In some such compositions or combinations, the multispecific anti-BCMA antibody or functional fragment thereof targets BCMA and CD3. In some such compositions or combinations, the multispecific anti-BCMA antibody or functional fragment thereof is anti-BCMA×CD3 bispecific antibody or functional fragment thereof. In some such compositions or combinations, the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B. In some such compositions or combinations, the anti-BCMA×CD3 bispecific antibody or functional fragment thereof comprises a first antigen-binding domain that specifically binds to BCMA comprising three heavy chain CDRs (HCDR1, HCDR2 and HCDR3) contained within a heavy chain variable region (HCVR) comprising the amino acid sequence of SEQ ID NO: 287, and three light chain CDRs (LCDR1, LCDR2 and LCDR3) contained within a light chain variable region (LCVR) comprising the amino acid sequence of SEQ ID NO: 303. In some such compositions or combinations, the first antigen-binding domain that specifically binds to BCMA comprises a HCDR1 comprising the amino acid sequence of SEQ ID NO: 289, a HCDR2 comprising the amino acid sequence of SEQ ID NO: 291, a HCDR3 comprising the amino acid sequence of SEQ ID NO: 293, a LCDR1 comprising the amino acid sequence of SEQ ID NO: 305, a LCDR2 comprising the amino acid sequence of SEQ ID NO: 307, and a LCDR3 comprising the amino acid sequence of SEQ ID NO: 309. In some such compositions or combinations, the anti-BCMA×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence selected from the group consisting of SEQ ID NOS: 311 and 319, and three light chain CDRs (LCDR1, LCDR2 and LCDR3) contained within a light chain variable region (LCVR) comprising the amino acid sequence of SEQ ID NO: 303. In some such compositions or combinations, the second antigen-binding domain that specifically binds to CD3 comprises a HCDR1 comprising the amino acid sequence of SEQ ID NO: 313 or 321, a HCDR2 comprising the amino acid sequence of SEQ ID NO: 315 or 323, a HCDR3 comprising the amino acid sequence of SEQ ID NO: 317 or 325, a LCDR1 comprising the amino acid sequence of SEQ ID NO: 305, a LCDR2 comprising the amino acid sequence of SEQ ID NO: 307, and a LCDR3 comprising the amino acid sequence of SEQ ID NO: 309. In some such compositions or combinations, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 289, 291, and 293, respectively, and LCDR1, LCDR2, and LCDR3 comprising the amino acid sequences of SEQ ID NOS: 305, 307, and 309, respectively; and (b) a second antigen-binding domain that comprises HCDR1, HCDR2, and HCDR3 comprising the amino acid sequences of SEQ ID NOS: 313, 315, and 317, respectively, and LCDR1, LCDR2, and LCDR3 comprising the amino acid sequences of SEQ ID NOS: 305, 307, and 309, respectively. In some such compositions or combinations, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 289, 291, and 293, respectively, and LCDR1, LCDR2, and LCDR3 comprising the amino acid sequences of SEQ ID NOS: 305, 307, and 309, respectively; and (b) a second antigen-binding domain that comprises HCDR1, HCDR2, and HCDR3 comprising the amino acid sequences of SEQ ID NOS: 321, 323, and 325, respectively, and LCDR1, LCDR2, and LCDR3 comprising the amino acid sequences of SEQ ID NOS: 305, 307, and 309, respectively. In some such compositions or combinations, the anti-BCMA×CD3 bispecific antibody or functional fragment thereof comprises a human IgG heavy chain constant region. In some such compositions or combinations, the human IgG heavy chain constant region is isotype IgG4 or IgG1. In some such compositions or combinations, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR).

In some such compositions or combinations, the plasma cell depleting agent is further in combination with an effective amount of a B cell depleting agent and/or an immunoglobulin depleting agent. In some such compositions or combinations, the plasma cell depleting agent is further in combination with an effective amount of a B cell depleting agent and an immunoglobulin depleting agent. In some such compositions or combinations, the B cell depleting agent is capable of depleting B cells and plasma cells that express low levels of BCMA. In some such compositions or combinations, the B cell depleting agent is an agent that binds to a B cell surface molecule. In some such compositions or combinations, the B cell depleting agent is selected from an anti-CD19 antibody, an anti-CD20 antibody, an anti-CD19 antibody and an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such compositions or combinations, the B cell depleting agent is an anti-CD20 antibody or a functional fragment thereof, wherein the anti-CD20 antibody is a multispecific antibody or a functional fragment thereof. In some such compositions or combinations, the multispecific anti-CD20 antibody or functional fragment thereof targets CD20 and CD3. In some such compositions or combinations, the multispecific anti-CD20 antibody or functional fragment thereof is anti-CD20×CD3 bispecific antibody or functional fragment thereof. In some such compositions or combinations, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 329, and three light chain CDRs (LCDR1, LCDR2 and LCDR3) contained within a light chain variable region (LCVR) comprising the amino acid sequence of SEQ ID NO: 330. In some such compositions or combinations, the first antigen-binding domain that specifically binds to CD20 comprises a HCDR1 comprising the amino acid sequence of SEQ ID NO: 332, a HCDR2 comprising the amino acid sequence of SEQ ID NO: 333, a HCDR3 comprising the amino acid sequence of SEQ ID NO: 334, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337. In some such compositions or combinations, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 331, and three light chain CDRs (LCDR1, LCDR2 and LCDR3) contained within a light chain variable region (LCVR) comprising the amino acid sequence of SEQ ID NO: 330. In some such compositions or combinations, the second antigen-binding domain that specifically binds to CD3 comprises a HCDR1 comprising the amino acid sequence of SEQ ID NO: 338, a HCDR2 comprising the amino acid sequence of SEQ ID NO: 339, a HCDR3 comprising the amino acid sequence of SEQ ID NO: 340, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337. In some such compositions or combinations, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 332, 333, and 334, respectively, and LCDR1, LCDR2, and LCDR3 comprising the amino acid sequences of SEQ ID NOS: 335, 336, and 337, respectively; and (b) a second antigen-binding domain that comprises HCDR1, HCDR2, and HCDR3 comprising the amino acid sequences of SEQ ID NOS: 338, 339, and 340, respectively, and LCDR1, LCDR2, and LCDR3 comprising the amino acid sequences of SEQ ID NOS: 335, 336, and 337, respectively. In some such compositions or combinations, the anti-CD20×CD3 bispecific antibody or functional fragment thereof comprises a human IgG heavy chain constant region. In some such compositions or combinations, the human IgG heavy chain constant region is isotype IgG4 or IgG1. In some such compositions or combinations, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such compositions or combinations, the B cell depleting agent is an agent targeting a B cell survival factor. In some such compositions or combinations, the B cell depleting agent is a BLyS/BAFF inhibitor, an APRIL inhibitor, a BLyS receptor 3/BAFF receptor inhibitor, or any combination thereof.

In some such compositions or combinations, the immunoglobulin depleting agent is capable of accelerating IgG clearance. In some such compositions or combinations, the immunoglobulin depleting agent is a neonatal Fc receptor (FcRn) blocker. In some such compositions or combinations, the FcRn blocker is selected from Efgartigimod (ARGX-113), Rozanolixizumab (UCB7665), Batoclimab (RVT-1401), IMVT-1402, Nipocalimab (M281), Orilanolimab (SYNT001), and any combinations thereof.

In some such compositions or combinations, the composition or combination is for use in a method of inserting a nucleic acid encoding a polypeptide of interest into a target genomic locus in a cell or a population of cells in a subject. In some such compositions or combinations, the composition or combination is for use in a method of expressing a polypeptide of interest from a target genomic locus in a cell or a population of cells in a subject. In some such compositions or combinations, the composition or combination is for use in a method of treating an enzyme deficiency in a subject in need thereof. In some such compositions or combinations, the composition or combination is for use in a method of preventing or reducing the onset of a sign or symptom of an enzyme deficiency in a subject in need thereof.

In another aspect, provided are kits comprising any of the above compositions or combinations. In another aspect, provided are any of the above CD40 inhibitors (e.g., CD40 antigen-binding molecules) for use in any of the above methods.

### BRIEF DESCRIPTION OF THE FIGURES

FIGS. 1A-1C show the effect of anti-CD40×CD40 bispecific antibodies on IL6 production in the presence of constant CD40L in human B cells from three different donors.

FIGS. 1D-1E show the effect of anti-CD40×CD40 bispecific antibodies on IL6 production in the presence of constant CD40L in human B cells from two different donors.

FIGS. 2A-2C show the effect of anti-CD40×CD40 bispecific antibodies on IL10 production in the presence of constant CD40L in human B cells from three different donors.

FIGS. 2D-2E show the effect of anti-CD40×CD40 bispecific antibodies on IL10 production in the presence of constant CD40L in human B cells from two different donors.

FIGS. 3A-3C show the effect of anti-CD40×CD40 bispecific antibodies on TNFα production in the presence of constant CD40L in human B cells from three different donors.

FIGS. 3D-3E show the effect of anti-CD40×CD40 bispecific antibodies on TNFα production in the presence of constant CD40L in human B cells from two different donors.

FIGS. 3F-3G show analysis of agonist activity of anti-CD40×CD40 bispecific antibodies as measured by IL6 production in human B cells from two different donors.

FIGS. 4A-4B show the effect of anti-CD40×CD40 bispecific antibodies on IL-12/IL-23p40 production in the presence of constant CD40L from human monocyte derived dendritic cells from two different donors.

FIGS. 5A-5B show analysis of agonist activity of anti-CD40×CD40 bispecific antibodies as measured by IL6 production in human B cells from two different donors.

FIGS. 6A-6B show analysis of agonist activity of anti-CD40×CD40 bispecific antibodies as measured by IL10 production in human B cells from two different donors.

FIG. 7 shows an experiment timeline using a NP-KLH immunization model as disclosed in Example 9.

FIG. 8A shows the effect of anti-CD40×CD40 bispecific antibodies on the frequency of NP positive germinal center B cells. * p<0.05.

FIG. 8B shows the effect of anti-CD40×CD40 bispecific antibodies on the frequency of NP IgG1 titers in mouse serum. * p<0.05; ** p<0.005.

FIG. 9A shows the effect of anti-CD40×CD40 bispecific antibodies on the frequency of NP positive germinal center B cells.

FIG. 9B shows the effect of anti-CD40×CD40 bispecific antibodies on the frequency of NP IgG1 titers in mouse serum.

FIG. 10 shows an experiment timeline using a mouse EAE model as disclosed in Example 10.

FIG. 11A shows mean EAE symptom scores for REGN16431- or REGN16432-treated mice.

FIG. 11B shows mean EAE symptom scores for REGN16334- or REGN16335-treated mice.

FIG. 11C shows percent of initial body weight in REGN16431- or REGN16432-treated mice.

FIG. 11D shows percent of initial body weight in REGN16334- or REGN16335-treated mice.

FIG. 12A shows mean EAE symptom scores for anti-CD40×CD40 bispecific antibody-treated mice.

FIG. 12B shows percent of initial body weight in anti-CD40×CD40 bispecific antibody-treated mice.

FIG. 13 shows an experiment timeline for the study described in Example 11.

FIG. 14 shows the effect of antibody-mediated CD40L blockade on the development of anti-AAV IgG titers following treatment with an AAV8 vector.

FIGS. 15A-15C show the effect of antibody-mediated CD40L blockade on transduction by a second AAV8 vector.

FIG. 16 shows an experiment timeline for the study described in Examples 12, 13, and 16.

FIGS. 17A-17B show the effect of antagonistic anti-CD40 antibodies on the development of anti-AAV IgG and IgM titers following treatment with an AAV8 vector.

FIGS. 18A-18C show the effect of antagonistic anti-CD40 antibodies on transduction by a second AAV8 vector.

FIG. 19 shows an experiment timeline for the study described in Examples 14 and 15.

FIGS. 20A-20D show the effect of antagonistic anti-CD40 antibodies on steady-state polyclonal and AAV-specific germinal center B cell responses.

FIGS. 21A-21E show the effect of antagonistic anti-CD40 antibodies on follicular T helper cell (TFH) responses and AAV-specific T cell responses.

FIG. 22 shows the effect of antagonistic anti-CD40 antibodies on the development of anti-transgene IgG following treatment with an AAV8 vector.

FIG. 23 shows a cryoEM reconstruction of CD40 in complex with the Fab arms 30027P2, 21519P2, and 21520P2.

FIG. 24 shows an experimental timeline for the study described in Examples 28, 29, 30, and 31.

FIG. 25 shows the effect of plasma cell depletion with anti-BCMA×CD3 bispecific antibody, FcRn blockade via efgartigimod alfa, B cell depletion with anti-CD19 and anti-CD20 antibodies (anti-CD19/CD20 antibodies), or combination thereof, on anti-AAV8 capsid IgG titers over time in mice previously treated with recombinant AAV8 vector.

FIG. 26 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second AAV8 vector in mice previously treated with recombinant AAV8 vector, as measured by Taqman quantitative real-time polymerase chain reaction (PCR) of green fluorescent protein (GFP) transgene DNA.

FIG. 27 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by Tagman quantitative real-time reverse-transcription PCR of GFP transgene RNA.

FIGS. 28A-28B show the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by GFP immunohistochemical (IHC) staining of formalin-fixed paraffin embedded liver sections. FIG. 28A shows GFP-positive area quantified using HALO software (Indica labs). FIG. 28B shows representative images.

FIGS. 29A-29J show flow cytometry analysis of B cell and plasma cell frequencies and counts in bone marrow and spleen following treatment with anti-BCMA×CD3 bispecific antibody, FcRn blockade, anti-CD19/CD20 antibodies, or combinations thereof. FIG. 29A shows bone marrow plasma cell frequencies, FIG. 29B shows spleen plasma cell frequencies, FIG. 29C shows spleen naive B cell frequencies, FIG. 29D shows spleen total memory B cell frequencies, FIG. 29E shows spleen AAV-specific memory B cell frequencies, FIG. 29F shows bone marrow plasma cell counts, FIG. 29G shows spleen plasma cell counts, FIG. 29H shows spleen naive B cell counts, FIG. 29I shows spleen total memory B cell counts, and FIG. 29J shows spleen AAV-specific memory B cell counts.

FIG. 30 shows the effect of efgartigimod on serum drug concentration of REGN5458 (BCMA×CD3).

FIG. 31 shows an experimental timeline for the study described in Example 32.

FIGS. 32A-32B show the effect of plasma cell depletion, B cell depletion, neonatal Fc receptor blockade, and combinations thereof, on naturally-occurring anti-AAV antibody titers in cynomolgus macaques. AAV8 neutralizing antibody (NAb) titer levels are presented for each treatment group over the duration of the study (FIG. 32A) and specifically at Study Day 29 (FIG. 32B).

FIG. 33 shows an experimental diagram for the non-human primate studies described in Examples 36 and 37.

FIGS. 34A-34F show the effect of prophylactic CD40 blockade on serum anti-AAV8 IgM, IgG, and neutralizing antibody titers in cynomolgus macaques.

FIGS. 35A-35C show the effect of prophylactic CD40 blockade on ability to systemically re-administer a second AAV8 vector in cynomolgus macaques.

FIGS. 36A-36C shows the effect of prophylactic CD40 blockade on T cell-induced liver injury and expression of an immunogenic GFP transgene in liver.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” or “wild-type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous APP sequence of an animal refers to a native APP sequence that naturally occurs at the APP locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline (tet)-regulated promoters (e.g., a tetracycline-responsive promoter, a tetracycline operator sequence (tetO), a tet-On promoter, or a tet-Off promoter), steroid-regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example, temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

The term “CD40,” as used herein, refers to cluster of differentiation 40 (CD40/TNFRSF5), a co-stimulatory cell surface receptor that is part of the tumor necrosis factor (TNF) receptor superfamily. In some embodiments, the CD40 is a human CD40. In some embodiments, the CD40 protein comprises the amino acid sequence of human CD40 set forth in UniProt Accession No. Q09LL4.

The term “antigen-binding molecule” includes antibodies and antigen-binding fragments of antibodies, including multispecific antibodies, e.g., bispecific antibodies.

The term “antibody,” as used herein, refers to an antigen-binding molecule or molecular complex comprising a set of complementarity determining regions (CDRs) that specifically bind to or interact with a particular antigen (e.g., CD40, BCMA, CD20, CD3). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, but are not limited to, the Kabat definition, the Chothia definition, the AbM definition (enhanced Chothia or Martin), the IMGT definition, and the Honneger definition (AHo). In general terms, the Kabat definition is based on sequence variability, the Chothia definition is based on the location of the structural loop regions, and the AbM definition is a compromise between the Kabat and Chothia approaches. See, e.g., Kabat et al., “Sequences of Proteins of Immunological Interest,” National Institutes of Health, Bethesda, Md. (1991); Chothia et al., J Mol Biol (1987), 4:901-17; Al-Lazikani et al., J. Mol. Biol. 273:927-948 (1997); and Martin et al., Proc. Natl. Acad. Sci. USA 86:9268-9272 (1989); see also Dondelinger et al., Front. Immunol. (2018), 9:2278, doi:10.3389/fimmu.2018.02278. Public databases are also available for identifying CDR sequences within an antibody.

The term “antibody,” as used herein, also includes antigen-binding fragments of full antibody molecules. The terms “antigen-binding portion” of an antibody, “antigen-binding fragment” of an antibody, “antigen-binding domain,” and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or heterodimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. In some embodiments, a multispecific antibody (e.g., bispecific antibody) has an arm that binds to a first epitope of an antigen and an arm that binds to a second epitope of the same antigen.

Any multispecific antibody format may be adapted for use in the context of an antibody or antigen-binding fragment of an antibody of the present disclosure using routine techniques available in the art. For example, the present disclosure includes bispecific antibodies wherein one arm of an immunoglobulin is specific for a first epitope of CD40, and the other arm of the immunoglobulin is specific for a second epitope of CD40. As another example, the present disclosure includes bispecific antibodies wherein one arm of an immunoglobulin is specific for a first epitope of BCMA or CD20, and the other arm of the immunoglobulin is specific for a second epitope of CD3. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency, and geometry. See, e.g., Kazane et al., J. Am. Chem. Soc. (Epub: Dec. 4, 2012).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant antibody,” as used herein, is intended to include all antibodies that are prepared, expressed, created, or isolated by recombinant means. The term includes, but is not limited to, antibodies expressed using a recombinant expression vector transfected into a host cell (e.g., Chinese hamster ovary (CHO) cell) or cellular expression system, antibodies isolated from a recombinant, combinatorial human antibody library, and antibodies isolated from a non-human animal (e.g., a mouse, such as a mouse that is transgenic for human immunoglobulin genes (see e.g., Taylor et al. (1992) Nucl. Acids Res. 20:6287-6295). In some embodiments, the recombinant antibody is a recombinant human antibody. In some embodiments, recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less, e.g., 10⁻⁷ M, 10⁻⁸ M, 10⁻⁹ M, 10⁻¹⁰ M, 10⁻¹¹ M, or 10⁻¹² M (a smaller K_{D} denotes a tighter binding). Methods for determining whether an antibody specifically binds to an antigen are known in the art and include, for example, equilibrium dialysis, surface plasmon resonance (e.g., BIACORE™), bio-layer interferometry assay (e.g., Octet® HTX biosensor), solution-affinity ELISA, and the like. In some embodiments, specific binding is measured in a surface plasmon resonance assay, e.g., at 25° C. or 37° C. An antibody or antigen-binding fragment that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example, using the BIACORE™ system (Cytiva, Marlborough, MA).

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or discontinuous (e.g., conformational). A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. An epitope typically includes at least 3, and more usually, e.g., at least 5 or at least 8-10 amino acids in a unique spatial conformation.

Methods for determining the epitope of an antigen-binding protein, e.g., an antibody or antigen-binding fragment, include alanine scanning mutational analysis, peptide blot analysis (Reineke, Methods Mol Biol 2004, 248:443-463), peptide cleavage analysis, crystallographic studies, and nuclear magnetic resonance (NMR) analysis. In addition, methods such as epitope exclusion, epitope extraction, and chemical modification of antigens can be employed (Tomer, Prot Sci 2000, 9:487-496). Another method that can be used to identify the amino acids within a polypeptide with which an antigen-binding protein (e.g., an antibody or antigen-binding fragment) interacts is hydrogen/deuterium exchange detected by mass spectrometry (HDX). See, e.g., Ehring, Analytical Biochemistry 1999, 267:252-259; Engen and Smith, Anal Chem 2001, 73:256A-265A.

The term “competes,” as used in reference to competing for binding, refers to an antigen-binding protein (e.g., antibody or antigen-binding fragment) that binds to an antigen and inhibits or blocks the binding of another antigen-binding protein (e.g., antibody or antigen-binding fragment) to the antigen. Unless otherwise stated, the term also includes competition between two antigen-binding proteins (e.g., antibodies) in both orientations, i.e., a first antigen that binds an antigen and blocks binding of the antigen by a second antibody and vice versa. Thus, in some embodiments, competition occurs in one such orientation. In some embodiments, the first antigen-binding protein (e.g., antibody) and second antigen-binding protein (e.g., antibody) may bind to the same epitope. Alternatively, the first and second antigen-binding proteins (e.g., antibodies) may bind to different epitopes, which may be overlapping or non-overlapping, wherein binding of one antigen-binding protein inhibits or blocks the binding of the second antigen-binding protein, e.g., via steric hindrance. Competition between antigen-binding proteins may be measured by methods known in the art, e.g., by a real-time, label-free bio-layer interferometry assay.

In the context of viruses in the present disclosure (e.g., AAV), the term “neutralizing antibody” or “nAb” refers to an antibody that binds to a virus and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 90%, e.g., at least 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and “substantially identical” mean that two peptide sequences, when optimally aligned, share at least about 90% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the disclosure to a database containing a large number of sequences from different organisms is the computer program BLAST, especially BLASTP or TBLASTN, using default parameters. See, e.g., Altschul et al., 1990, J. Mol. Biol. 215: 403-410 and 1997 Nucleic Acids Res. 25:3389-3402.

A “variant” of a polypeptide, such an immunoglobulin, VH, VL, heavy chain, light chain, or CDR comprising an amino acid sequence specifically set forth herein, refers to a polypeptide comprising an amino acid sequence that is at least about 70%-99.9% (e.g., at least 70, 75, 80, 85, 90, 91, 92, 93, 94, 95, 96, 97, 98, 99, 99.5, or 99.9%) identical to the reference polypeptide sequence (e.g., as set forth in the sequence listing below), when the comparison is performed by a BLAST algorithm wherein the parameters of the algorithm are selected to give the largest match between the respective sequences over the entire length of the respective reference sequences. In some embodiments, a variant of a polypeptide includes a polypeptide having the amino acid sequence of a reference polypeptide sequence (e.g., as set forth in the sequence listing below) but for one or more (e.g., 1 to 10, or less than 20, or less than 10) missense mutations (e.g., conservative substitutions), nonsense mutations, deletions, or insertions.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p<0.05.

In the event of a conflict between a sequence in the application and an indicated accession number or position in an accession number, the sequence in the application predominates.

### DETAILED DESCRIPTION

### I. Overview

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency (e.g., FIX deficiency or GAA deficiency) in a subject in need thereof, and methods of preventing or reducing the onset of a sign or symptom of an enzyme deficiency in a subject in need thereof.

The methods use CD40 inhibitors (e.g., CD40 antigen-binding molecules) (e.g., CD40×CD40 bispecific antigen-binding molecules disclosed herein) to mitigate immune response and facilitate redosing of nucleic acid constructs encoding a polypeptide of interest and nuclease agents targeting a target genomic locus (e.g., in a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV). The CD40 inhibitors (e.g., CD40 antigen-binding molecules) can be used for inhibiting CD40L-induced activation of CD40, e.g., in or on a cell that expresses CD40 (e.g., a B cell, dendritic cell, monocyte, platelet, or macrophage). The CD40 inhibitors (e.g., CD40 antigen-binding molecules) are able to suppress host B cell responses to new antigens. In AAV gene therapies, seronegative/naive patients are dosed with AAV and develop antibody responses to the AAV capsid antigen. This antibody response prevents future re-dosing of AAV because the antibodies are neutralizing, and the antibody response is sustained for 10+ years. When AAV is co-administered with CD40 inhibitors (e.g., CD40 antigen-binding molecules), the B cell response is suppressed and anti-AAV IgG responses are significantly suppressed. When CD40 inhibitor (e.g., CD40 antigen-binding molecule) is given during the period of AAV antigen exposure, the anti-AAV antibody response can be suppressed in animals. This allows re-dosing of any AAV gene therapy product. For example, for CRISPR-mediated gene insertion platforms consisting of an AAV and LNP, the AAV and/or LNP can be re-dosed multiple times when CD40 inhibitor (e.g., CD40 antigen-binding molecule) is co-administered. The CD40 inhibitor (e.g., CD40 antigen-binding molecule) prevents antibody formation against the AAV. The CD40 inhibitor (e.g., CD40 antigen-binding molecule) can also prevent antibody formation against certain LNP components (e.g., anti-PEG IgG), which can improve efficacy of LNP redosing.

Using CD40 inhibitors (e.g., CD40 antigen-binding molecules) to mitigate an anti-AAV antibody response can allow for repeated dosing of an identical gene insertion therapeutic cargo. This allows targeted cells in a subject to produce a polypeptide of interest in a step-wise, increasing fashion due to increased gene insertion in additional targeted cells following repeated dosing until a desired level of expression and/or activity of the polypeptide of interest is achieved in a subject without overshooting. This can be particularly advantageous in situations in which overshooting (i.e., achieving higher than desired levels of expression and/or activity of the polypeptide of interest) would result in undesired side effects (e.g., toxicity). Likewise, using CD40 inhibitors (e.g., CD40 antigen-binding molecules) to mitigate an anti-AAV antibody response can allow for gene insertion of an AAV template into two separate genomic locations from two discrete dosings (e.g., two discrete dosings of AAV and LNP). Similarly, using CD40 inhibitors (e.g., CD40 antigen-binding molecules) to mitigate an anti-AAV antibody response can allow for gene insertion of two different AAV templates (encoding different polypeptides of interest or the same polypeptide of interest) from two discrete dosings (e.g., two discrete dosings of AAV and LNP).

Other broad-spectrum immunosuppression methodologies have not been shown to be effective at enabling AAV vector re-administration at levels equivalent to naive individuals. Similarly, CD40L blockade has also been shown to enable only partial AAV re-transduction. The CD40 antigen-binding molecules disclosed herein, which show superior binding characteristics and performance with low agonism as compared to other CD40 antigen-binding molecules, can achieve levels of re-transduction similar to naive animals. In addition, single agent CD40 blockade is more targeted than other immunosuppression methods because it partially preserves de novo T-cell-independent antibody responses, which may provide patients full or partial protection from opportunistic infections in the absence of a de novo T-cell-dependent antibody response.

The methods using CD40 inhibitors can also use plasma cell depleting agents or combinations comprising plasma cell depleting agents when a subject has preexisting immunity against an immunogen to be administered to the subject (e.g., an immunogenic delivery vehicle such as, e.g., AAV). For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. Examples of such methods include the following: (1) a plasma cell depleting agent or a combination comprising a plasma cell depleting agent as disclosed herein [e.g., in combination with a B cell depleting agent (e.g., a CD20×CD3 antigen-binding molecule) and/or an immunoglobulin depleting agent and/or plasmapheresis, therapeutic plasma exchange, or immunoadsorption] is used to eliminate preexisting immunity to an immunogen (e.g., AAV), while a CD40 inhibitor is used to prevent any new antibody response to the immunogen on subsequent immunogen exposure; (2) a plasma cell depleting agent or a combination comprising a plasma cell depleting agent as disclosed herein is used to eliminate potential residual antibody responses generated following immunogen (e.g., AAV) exposure in the presence of CD40 blockade (e.g., if CD40 blockade is not completely effective); or (3) CD40 blockade is used concurrently with a plasma cell depleting agent or a combination comprising a plasma cell depleting agent as disclosed herein to block ongoing antibody responses to an immunogen (e.g., AAV) from recent exposure. The present disclosure provides, for example, a distinct B cell immunosuppression approach that enables AAV vector re-transduction in subjects with preexisting AAV immunity at levels equal to seronegative subjects, by depleting pre-existing nAbs (e.g., via combined plasma cell and immunoglobulin depletion). Long-lived plasma cells (LLPC) mediate constitutive antibody production to most antigens and are the likely reservoir of persistent anti-AAV antibody immunity. It was discovered that pre-existing AAV nAbs in subjects with preexisting AAV immunity could be directly eliminated in vivo by LLPC depletion with linvoseltamab, a fully-human T cell-bridging bispecific antibody targeting B cell maturation antigen and CD3 (anti-BCMA×CD3 bispecific antibody), either alone or in combination with B cell depletion (to eliminate non-LLPC sources of anti-AAV nAbs) and/or FcRn blockade (to accelerate serum IgG clearance). Such methods can use plasma cell depleting agents or combinations comprising plasma cell depleting agents to mitigate immune response in subjects with preexisting immunity and facilitate redosing of nucleic acid constructs encoding a polypeptide of interest and nuclease agents targeting a target genomic locus. Optionally, the plasma cell depleting agents (e.g., BCMA×CD3 antigen-binding molecules) are used in combination with other immunosuppression methodologies, such as immunoglobulin depleting agents (e.g., FcRn blockers or IgG degrading enzymes), B cell depleting agents, plasmapheresis, therapeutic plasma exchange, immunoadsorption, broad spectrum immunosuppression, or combinations thereof. In one example, plasma cell depleting agents (e.g., BCMA×CD3 bispecific antigen-binding molecules) are used in combination with immunoglobulin depleting agents (e.g., IgG half-life reducers, such as FcRn blockers). In another example, plasma cell depleting agents (e.g., BCMA×CD3 bispecific antigen-binding molecules) are used in combination with B cell depleting agents (e.g., CD20×CD3 antigen-binding molecules). In another example, BCMA×CD3 bispecific antigen-binding molecules are used in combination with immunoglobulin depleting agents (e.g., FcRn blockers) and B cell depleting agents (e.g., CD20×CD3 antigen-binding molecules). This allows re-dosing of any AAV gene therapy product in subjects with preexisting AAV immunity. For example, for CRISPR-mediated gene insertion platforms consisting of an AAV and LNP, the AAV and/or LNP can be re-dosed multiple times in subjects with preexisting AAV immunity when plasma cell depleting agents or combinations comprising plasma cell depleting agents are co-administered.

Using plasma cell depleting agents or combinations comprising plasma cell depleting agents to mitigate an anti-AAV antibody response in subjects with preexisting AAV immunity can allow for repeated dosing of an identical gene insertion therapeutic cargo. This allows targeted cells in a subject to produce a polypeptide of interest in a step-wise, increasing fashion due to increased gene insertion in additional targeted cells following repeated dosing until a desired level of expression and/or activity of the polypeptide of interest is achieved in a subject without overshooting. This can be particularly advantageous in situations in which overshooting (i.e., achieving higher than desired levels of expression and/or activity of the polypeptide of interest) would result in undesired side effects (e.g., toxicity). Likewise, using plasma cell depleting agents or combinations comprising plasma cell depleting agents to mitigate an anti-AAV antibody response in subjects with preexisting AAV immunity can allow for gene insertion of an AAV template into two separate genomic locations from two discrete dosings (e.g., two discrete dosings of AAV and LNP). Similarly, using plasma cell depleting agents or combinations comprising plasma cell depleting agents to mitigate an anti-AAV antibody response in subjects with preexisting AAV immunity can allow for gene insertion of two different AAV templates (encoding different polypeptides of interest or the same polypeptide of interest) from two discrete dosings (e.g., two discrete dosings of AAV and LNP).

Also provided are compositions, combinations, or kits comprising a CD40 inhibitor (e.g., CD40 antigen-binding molecule) in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus. As used herein, the term “in combination with” means that additional component(s) may be administered prior to, concurrent with, or after the administration of the CD40 inhibitor (e.g., CD40 antigen-binding molecule). The different components of the combination can be formulated into a single composition, e.g., for simultaneous delivery, or formulated separately into two or more compositions (e.g., a kit including each component, for example, wherein the further agent is in a separate formulation).

Also provided are compositions or combinations or kits comprising a plasma cell depleting agent or combination comprising a plasma cell depleting agent in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus. Such compositions, combinations, or kits comprise the plasma cell depleting agent in combination with a CD40 inhibitor. As used herein, the term “in combination with,” for example, a plasma cell depleting agent means that additional component(s) may be administered prior to, concurrent with, or after the administration of the plasma cell depleting agent. The different components of the combination can be formulated into a single composition, e.g., for simultaneous delivery, or formulated separately into two or more compositions (e.g., a kit including each component, for example, wherein the further agent is in a separate formulation).

### II. CD40 Inhibitors and CD40 Antigen-Binding Molecules

In one aspect, the present disclosure relates to CD40 inhibitors. CD40 inhibitors can block or suppress CD40 and/or CD40-mediated cellular signaling pathways and/or mechanisms, e.g., cellular signaling pathways and/or mechanisms associated with an immune response. CD40 inhibitors can include antigen-binding molecules such as monospecific, bispecific, and multispecific antibodies that bind to CD40 (e.g., antagonist antigen-binding molecules, including monospecific, bispecific, and multispecific antibodies, that bind to CD40) or other CD40-CD40L inhibitors such as CD40L antigen-binding molecules (e.g., antibodies), peptide inhibitors, or small molecule inhibitors. Examples of such inhibitors are disclosed elsewhere herein. In some embodiments, the antigen-binding molecule is a monospecific anti-CD40 antibody. In some embodiments, the antigen-binding molecule is a multispecific (e.g., bispecific) antibody. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. In some embodiments, the CD40 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second binding specificity. In some embodiments, the multispecific antibody contains an antigen-binding domain that is specific for CD40 and an antigen-binding domain that is specific for another antigen (i.e., not CD40). In some embodiments, the multispecific antibody contains an antigen-binding domain that is specific for a first epitope of CD40 and an antigen-binding domain that is specific for a second epitope of CD40.

A CD40 inhibitor can be used for inhibiting CD40L-induced activation of CD40, e.g., in or on a cell that expresses CD40 (e.g., a B cell, dendritic cell, monocyte, platelet, or macrophage). The CD40 inhibitors are able to suppress host B cell responses to new antigens. In AAV gene therapies, seronegative/naive subjects are dosed with AAV and develop antibody responses to the AAV capsid antigen. This antibody response prevents future re-dosing of AAV because the antibodies are neutralizing, and the antibody response is sustained for 10+ years. When AAV is co-administered with a CD40 inhibitor, the B cell response is suppressed and anti-AAV IgG responses are significantly suppressed. When a CD40 inhibitor is given during the period of AAV antigen exposure, the anti-AAV antibody response can be suppressed in animals. This allows re-dosing of any AAV gene therapy product.

In some embodiments, a CD40 inhibitor is administered to a subject not having a pre-existing immunity against an immunogen (e.g., an immunogenic delivery vehicle such as, e.g., AAV (e.g., AAV comprising a nucleic acid construct described herein)). In some embodiments, a CD40 inhibitor is administered to a subject not having a pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein. In some embodiments, a CD40 inhibitor is administered to a subject not having a pre-existing immunity against an AAV vector comprising a nucleic acid construct described herein.

In some embodiments, CD40 blockade using a CD40 inhibitor can prevent antibody and T cell responses to Cas proteins (e.g., Cas9 proteins) that might lead to transduced cell clearance and/or inflammation associated with a T cell response.

### A. Monospecific Anti-CD40 Antibodies

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within an HCVR/LCVR amino acid sequence pair selected from the group consisting of SEQ ID NOS: 2/10, 22/10, and 32/10. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment thereof comprises: (a) an HCDR1 comprising an amino acid sequence selected from the group consisting of SEQ ID NOS: 4, 24, and 34; (b) an HCDR2 comprising an amino acid sequence selected from the group consisting of SEQ ID NOS: 6, 26, and 36; (c) an HCDR3 comprising an amino acid sequence selected from the group consisting of SEQ ID NOS: 8, 28, and 38; (d) an LCDR1 comprising the amino acid sequence of SEQ ID NO: 12; (e) an LCDR2 comprising the amino acid sequence AAS (SEQ ID NO: 14); and (f) an LCDR3 comprising the amino acid sequence of SEQ ID NO: 16.

In some embodiments, the anti-CD40 antibody or antigen-binding fragment comprises: (a) an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; or (b) an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16; or (c) an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some embodiments, the anti-CD40 antibody comprises an HCDR1 comprising the amino acid sequence of SEQ ID NO: 4, an HCDR2 comprising the amino acid sequence of SEQ ID NO: 6, an HCDR3 comprising the amino acid sequence of SEQ ID NO: 8, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the anti-CD40 antibody comprises an HCDR1 consisting of the amino acid sequence of SEQ ID NO: 4, an HCDR2 consisting of the amino acid sequence of SEQ ID NO: 6, an HCDR3 consisting of the amino acid sequence of SEQ ID NO: 8, an LCDR1 consisting of the amino acid sequence of SEQ ID NO: 12, an LCDR2 consisting of the amino acid sequence AAS (SEQ ID NO: 14), and an LCDR3 consisting of the amino acid sequence of SEQ ID NO: 16.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 2; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the anti-CD40 antibody comprises an HCVR comprising or consisting of the amino acid sequence of SEQ ID NO: 2. In some embodiments, the anti-CD40 antibody comprises an LCVR comprising or consisting of the amino acid sequence of SEQ ID NO: 10.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 18; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the anti-CD40 antibody comprises a heavy chain comprising or consisting of the amino acid sequence of SEQ ID NO: 18. In some embodiments, the anti-CD40 antibody comprises a light chain comprising or consisting of the amino acid sequence of SEQ ID NO: 20.

In some embodiments, the anti-CD40 antibody comprises an HCDR1 comprising the amino acid sequence of SEQ ID NO: 24, an HCDR2 comprising the amino acid sequence of SEQ ID NO: 26, an HCDR3 comprising the amino acid sequence of SEQ ID NO: 28, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the anti-CD40 antibody comprises an HCDR1 consisting of the amino acid sequence of SEQ ID NO: 24, an HCDR2 consisting of the amino acid sequence of SEQ ID NO: 26, an HCDR3 consisting of the amino acid sequence of SEQ ID NO: 28, an LCDR1 consisting of the amino acid sequence of SEQ ID NO: 12, an LCDR2 consisting of the amino acid sequence AAS (SEQ ID NO: 14), and an LCDR3 consisting of the amino acid sequence of SEQ ID NO: 16.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 22; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the anti-CD40 antibody comprises an HCVR comprising or consisting of the amino acid sequence of SEQ ID NO: 22. In some embodiments, the anti-CD40 antibody comprises an LCVR comprising or consisting of the amino acid sequence of SEQ ID NO: 10.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 30; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the anti-CD40 antibody comprises a heavy chain comprising or consisting of the amino acid sequence of SEQ ID NO: 30. In some embodiments, the anti-CD40 antibody comprises a light chain comprising or consisting of the amino acid sequence of SEQ ID NO: 20.

In some embodiments, the anti-CD40 antibody comprises an HCDR1 comprising the amino acid sequence of SEQ ID NO: 34, an HCDR2 comprising the amino acid sequence of SEQ ID NO: 36, an HCDR3 comprising the amino acid sequence of SEQ ID NO: 38, an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the anti-CD40 antibody comprises an HCDR1 consisting of the amino acid sequence of SEQ ID NO: 34, an HCDR2 consisting of the amino acid sequence of SEQ ID NO: 36, an HCDR3 consisting of the amino acid sequence of SEQ ID NO: 38, an LCDR1 consisting of the amino acid sequence of SEQ ID NO: 12, an LCDR2 consisting of the amino acid sequence AAS (SEQ ID NO: 14), and an LCDR3 consisting of the amino acid sequence of SEQ ID NO: 16.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 32; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the anti-CD40 antibody comprises an HCVR comprising or consisting of the amino acid sequence of SEQ ID NO: 32. In some embodiments, the anti-CD40 antibody comprises an LCVR comprising or consisting of the amino acid sequence of SEQ ID NO: 10.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 40; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the anti-CD40 antibody comprises a heavy chain comprising or consisting of the amino acid sequence of SEQ ID NO: 40. In some embodiments, the anti-CD40 antibody comprises a light chain comprising or consisting of the amino acid sequence of SEQ ID NO: 20.

In some embodiments, the anti-CD40 antibody has at least 85% sequence identity, e.g., at least 90%, at least 91%, at least 92%, at least 93%, at least 94%, at least 95%, at least 96%, at least 97%, at least 98% or at least 99% sequence identity, to 30027P2. In some embodiments, the anti-CD40 antibody has the amino acid sequence of 30027P2.

In some embodiments, the anti-CD40 antibody has at least 85% sequence identity, e.g., at least 90%, at least 91%, at least 92%, at least 93%, at least 94%, at least 95%, at least 96%, at least 97%, at least 98% or at least 99% sequence identity, to 21519P2. In some embodiments, the anti-CD40 antibody has the amino acid sequence of 21519P2.

In some embodiments, the anti-CD40 antibody has at least 85% sequence identity, e.g., at least 90%, at least 91%, at least 92%, at least 93%, at least 94%, at least 95%, at least 96%, at least 97%, at least 98% or at least 99% sequence identity, to 21520P2. In some embodiments, the anti-CD40 antibody comprises or consists of the amino acid sequence of 21520P2.

Other CD40 antigen-binding molecules that can be used in the compositions, combinations, kits, or methods disclosed herein include other known anti-CD40 antibodies. Non-limiting examples of additional anti-CD40 antibodies include CD40 Monoclonal Antibody (1C10) (eBioscience™), CD40 Monoclonal Antibody (HM40-3) (eBioscience™), CD40 Monoclonal Antibody (5C3) (eBioscience™), CD40 Monoclonal Antibody (9G10) (Invitrogen), CD40 Monoclonal Antibody (3/23) (Invitrogen), CD40 Monoclonal Antibody (HB14) (Invitrogen), CD40 Polyclonal Antibody (Invitrogen), CD40 Monoclonal Antibody (OTI1F12), TrueMAB™ (OriGene), CD40 Recombinant Rabbit Monoclonal Antibody (5V8X7) (Invitrogen), CD40 Monoclonal Antibody (LOB7/6) (Invitrogen), CD40 Monoclonal Antibody (5C3) (Invitrogen), CD40 Monoclonal Antibody (HI40a) (Invitrogen), CD40 Monoclonal Antibody (IL-A156) (Invitrogen), CD40 Monoclonal Antibody (C7) (Invitrogen), CD40 Polyclonal Antibody (Invitrogen, e.g., Cat #PA5-111025, Cat #PA5-111024, Cat #PA5-109301, Cat #PA5-117850, Cat #PA5-27419, Cat #PA5-78980, Cat #PA1-31075), CD40 Monoclonal Antibody (3/23) (Invitrogen), CD40 Monoclonal Antibody (HB14) (Invitrogen), CD40 Recombinant Rabbit Monoclonal Antibody (Bethyl Laboratories), CD40 Monoclonal Antibody (OTI8B8), TrueMAB™ (OriGene), CD40 Monoclonal Antibody (OTI1F12), TrueMAB™ (OriGene), CD40 Monoclonal Antibody (OTI5C9), TrueMAB™ (OriGene), CD40 Monoclonal Antibody (UMAB255), UltraMAB™ (OriGene), CD40 Monoclonal Antibody (2A8G5) (Proteintech), CD40 Monoclonal Antibody (G28.5) (Proteintech), CD40 Monoclonal Antibody (1C10) (Proteintech), CD40 Monoclonal Antibody (G28.5) (Proteintech), CD40 Monoclonal Antibody (2A8G5) (Proteintech), CD40 Monoclonal Antibody (UMAB183), UltraMAB™ (OriGene), CD40 Monoclonal Antibody (UMAB183), UltraMAB™ (OriGene), CD40 Monoclonal Antibody (HB14) (AbboMax), CD40 Monoclonal Antibody (1G1) (Abnova), CD40 Polyclonal Antibody (AbboMax, Cat #500-3704), CD40 Monoclonal Antibody (FGK45) (Leinco Technologies), CD40 Monoclonal Antibody (3D9) (Abnova), and CD40 Monoclonal Antibody (2H8) (Abnova).

In some embodiments, the anti-CD40 antibody is an antagonistic anti-CD40 antibody or a functional fragment thereof. The antagonistic anti-CD40 antibody may comprise any antagonistic anti-CD40 antibody described above or known in the art. Non-limiting examples of additional antagonistic anti-CD40 antibodies include iscalimab (also known as CFZ533) disclosed in Kahaly et al. (2019) J. Endocr. Sco. 3:doi.org/10.1210/js.2019-0R19-6, Fisher et al. (2017) Arthritis Rheumatol. 69:1784, Farkash et al. (2019) Am. J. Transplant. 19:632, U.S. Pat. No. 8,828,396, International Patent Application Publication No. WO 2012/075111, and clinical trial NCT02291029 sponsored by Novartis Pharmaceuticals; ravagalimab (also known as ABBV-323 and Ab102) disclosed in International Patent Application Publication No. WO 2016/196314 and U.S. Patent Application Publication No. US 2022/0289858; BI-655064 disclosed in Visvannathan et al. (2016) Arthritis Rheumatol. 68:1588, U.S. Pat. No. 8,591,900, and clinical trial NCT03385564 sponsored by Boehringer Ingelheim; bleselumab (also known as ASKP1240 or 341G2) disclosed in Anil et al. (2018) Biopharm. Drug Dispos. 39:245-255, Harland et al. (2017) Am. J. Transplant. 17:159-171, U.S. Pat. Nos. 8,716,451 and 8,568,725, and clinical trials NCT01585233 and NCT02921789 sponsored by Astellas Pharma; ch5D12 disclosed in Kasran et al. (2005) Aliment. Pharmacol. Ther. 22:111-122 and U.S. Patent Application Publication No. US 2008/0085531; lucatumumab (also known as HCD122 or CHIR-12.12) disclosed in Bensinger et al. (2012) British J. Haematology 159:58-66, Byrd et al. (2012) Leuk. Lymphoma 53:10.3109/10428194.2012.681655, International Patent Application Publication No. WO 2005/044854, U.S. Patent Application Publication No. US 2007/0110754 and U.S. Pat. No. 8,828,396; CHIR-5.9 disclosed in International Patent Application Publication No. WO 2005/044854 and U.S. Pat. No. 8,637,032; abiprubart (KPL-404) disclosed in clinical trial NCT04497662 sponsored by Kiniksa Pharmaceuticals, Ltd. as well as in U.S. Patent Application Publication Nos. US 2023/0287132, US 2023/0203179, US 2023/0183367, and US 2023/0279135; BlIB063 disclosed in Musselli et al. (2017) 2017 ACR/ARHP Annual Meeting Abstract and International Patent Application Publication No. WO 2016028810; V19 and V15 disclosed in U.S. Patent Application Publication No. US 2022/0135694; h2C10 and variants thereof disclosed in U.S. Pat. No. 11,439,706; FFP104 (also known as PG102) disclosed in U.S. Pat. Nos. 8,669,352 and 11,396,552, International Patent Application Publication No. WO 2001/024823, U.S. Patent Application Publication No. US 2008/0085531, Bankert et al. (2015) J. Immunol. 194:4319-4327, and clinical trials NCT02193360 and NCT02465944 sponsored by Fast Forward Pharmaceuticals; Ab101 disclosed in U.S. Patent Application Publication No. US 2022/0289858; Antibody A, antibody B, Antibody C, disclosed in U.S. Pat. No. 11,242,394; G28.5 disclosed in International Patent Application Publication No. WO 2016028810; BMS3h-37, BMS3h-38, BMS3h-56, and BMS3h-198 disclosed in International Patent Application Publication No. WO2012145673A1, and Y12XX-hz28 (Vh-hzl4;Vk-hz2), Y12XX-hz40 (Vh-hzl2;Vk-hz3), and Y12XX-hz42 (Vh-hzl4;Vk-hz3) disclosed in International Patent Application Publication No. WO2020/106620A1, the contents of each of which are herein incorporated by reference in their entirety. An additional CD40 antibody useful in certain embodiments of the methods and compositions provided herein is teneliximab. Additional CD40 antagonist antibodies useful in certain embodiments of the methods and compositions provided herein are disclosed in, for example, International Patent Application Publication Nos. WO 02/11763, WO 02/28481, WO 03/045978, WO 03/029296, WO 03/028809, WO 2005/044854, WO 2006/073443, WO 2007/124299, WO 2011/123489, WO 2016/196,314, WO 2017/040566, WO 2017/060242, WO 2018/217976, WO2019/156565, WO 2020/144605, WO 2020/106620, WO 2020/006347, U.S. Patent Application Publication Nos. US 2020/0291123, US 2017/0158771, US 2008/0057070, and U.S. Pat. Nos. 5,874,082, 7,063,845, 9,125,893, 8,669,352, 9,598,494, 11,254,750, 11,780,927, 11,220,550, 11,202,827, 10,111,958, 11,242,397, 8,591,900, 9,475,879, 10,174,121, the contents of each of which are herein incorporated by reference in their entirety.

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 183/184. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 177, an HCDR2 comprising the amino acid sequence of SEQ ID NO: 178, an HCDR3 comprising the amino acid sequence of SEQ ID NO: 179, an LCDR1 comprising the amino acid sequence of SEQ ID NO: 180, an LCDR2 comprising the amino acid sequence of SEQ ID NO: 181, and an LCDR3 comprising the amino acid sequence of SEQ ID NO: 182.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 183; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 184. In some embodiments, the anti-CD40 antibody comprises an HCVR comprising or consisting of the amino acid sequence of SEQ ID NO: 183. In some embodiments, the anti-CD40 antibody comprises an LCVR comprising or consisting of the amino acid sequence of SEQ ID NO: 184.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 185; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 186. In some embodiments, the anti-CD40 antibody comprises a heavy chain comprising or consisting of the amino acid sequence of SEQ ID NO: 185. In some embodiments, the anti-CD40 antibody comprises a light chain comprising or consisting of the amino acid sequence of SEQ ID NO: 186. In some embodiments, the anti-CD40 antibody is abiprubart (also known as KPL-404).
Abiprubart (KPL-404) HCDR1=SEQ ID NO: 177
Abiprubart (KPL-404) HCDR2=SEQ ID NO: 178
Abiprubart (KPL-404) HCDR3=SEQ ID NO: 179
Abiprubart (KPL-404) LCDR1=SEQ ID NO: 180
Abiprubart (KPL-404) LCDR2=SEQ ID NO: 181
Abiprubart (KPL-404) LCDR3=SEQ ID NO: 182
Abiprubart (KPL-404) HCVR=SEQ ID NO: 183
Abiprubart (KPL-404) LCVR=SEQ ID NO: 184
Abiprubart (KPL-404) HC=SEQ ID NO: 185
Abiprubart (KPL-404) LC=SEQ ID NO: 186

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 141/142. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 135, an HCDR2 comprising the amino acid sequence of SEQ ID NO: 136, an HCDR3 comprising the amino acid sequence of SEQ ID NO: 137, an LCDR1 comprising the amino acid sequence of SEQ ID NO: 138, an LCDR2 comprising the amino acid sequence of SEQ ID NO: 139, and an LCDR3 comprising the amino acid sequence of SEQ ID NO: 140.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 141; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 142. In some embodiments, the anti-CD40 antibody comprises an HCVR comprising or consisting of the amino acid sequence of SEQ ID NO: 141. In some embodiments, the anti-CD40 antibody comprises an LCVR comprising or consisting of the amino acid sequence of SEQ ID NO: 142.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 143; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 144. In some embodiments, the anti-CD40 antibody comprises a heavy chain comprising or consisting of the amino acid sequence of SEQ ID NO: 143. In some embodiments, the anti-CD40 antibody comprises a light chain comprising or consisting of the amino acid sequence of SEQ ID NO: 144. In some embodiments, the anti-CD40 antibody is iscalimab.
Iscalimab (CFZ533) HCDR1=SEQ ID NO: 135
Iscalimab (CFZ533) HCDR2=SEQ ID NO: 136
Iscalimab (CFZ533) HCDR3=SEQ ID NO: 137
Iscalimab (CFZ533) LCDR1=SEQ ID NO: 138
Iscalimab (CFZ533) LCDR2=SEQ ID NO: 139
Iscalimab (CFZ533) LCDR3=SEQ ID NO: 140
Iscalimab (CFZ533) HCVR=SEQ ID NO: 141
Iscalimab (CFZ533) LCVR=SEQ ID NO: 142
Iscalimab (CFZ533) HC=SEQ ID NO: 143
Iscalimab (CFZ533) LC=SEQ ID NO: 144

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 151/268. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 145, an HCDR2 comprising the amino acid sequence of SEQ ID NO: 146, an HCDR3 comprising the amino acid sequence of SEQ ID NO: 147, an LCDR1 comprising the amino acid sequence of SEQ ID NO: 148, an LCDR2 comprising the amino acid sequence of SEQ ID NO: 149, and an LCDR3 comprising the amino acid sequence of SEQ ID NO: 150.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 151; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 268. In some embodiments, the anti-CD40 antibody comprises an HCVR comprising or consisting of the amino acid sequence of SEQ ID NO: 151. In some embodiments, the anti-CD40 antibody comprises an LCVR comprising or consisting of the amino acid sequence of SEQ ID NO: 268.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 152; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 153. In some embodiments, the anti-CD40 antibody comprises a heavy chain comprising or consisting of the amino acid sequence of SEQ ID NO: 152. In some embodiments, the anti-CD40 antibody comprises a light chain comprising or consisting of the amino acid sequence of SEQ ID NO: 153. In some embodiments, the anti-CD40 antibody is ravagalimab.
Ravagalimab (ABBV-323; Ab102) HCDR1=SEQ ID NO: 145
Ravagalimab (ABBV-323; Ab102) HCDR2=SEQ ID NO: 146
Ravagalimab (ABBV-323; Ab102) HCDR3=SEQ ID NO: 147
Ravagalimab (ABBV-323; Ab102) LCDR1=SEQ ID NO: 148
Ravagalimab (ABBV-323; Ab102) LCDR2=SEQ ID NO: 149
Ravagalimab (ABBV-323; Ab102) LCDR3=SEQ ID NO: 150
Ravagalimab (ABBV-323; Ab102) HCVR=SEQ ID NO: 151
Ravagalimab (ABBV-323; Ab102) LCVR=SEQ ID NO: 268
Ravagalimab (ABBV-323; Ab102) HC=SEQ ID NO: 152
Ravagalimab (ABBV-323; Ab102) LC=SEQ ID NO: 153

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 231/232. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 269, an HCDR2 comprising the amino acid sequence of SEQ ID NO: 220, an HCDR3 comprising the amino acid sequence of SEQ ID NO: 221, an LCDR1 comprising the amino acid sequence of SEQ ID NO: 222, an LCDR2 comprising the amino acid sequence of SEQ ID NO: 223, and an LCDR3 comprising the amino acid sequence of SEQ ID NO: 150.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 231; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 232. In some embodiments, the anti-CD40 antibody comprises an HCVR comprising or consisting of the amino acid sequence of SEQ ID NO: 231. In some embodiments, the anti-CD40 antibody comprises an LCVR comprising or consisting of the amino acid sequence of SEQ ID NO: 232.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 236; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 237. In some embodiments, the anti-CD40 antibody comprises a heavy chain comprising or consisting of the amino acid sequence of SEQ ID NO: 236. In some embodiments, the anti-CD40 antibody comprises a light chain comprising or consisting of the amino acid sequence of SEQ ID NO: 237. In some embodiments, the anti-CD40 antibody is BI-655064.
BI-655064 HCDR1=SEQ ID NO: 269
BI-655064 HCDR2=SEQ ID NO: 220
BI-655064 HCDR3=SEQ ID NO: 221
BI-655064 LCDR1=SEQ ID NO: 222
BI-655064 LCDR2=SEQ ID NO: 223
BI-655064 LCDR3=SEQ ID NO: 150
BI-655064 HCVR=SEQ ID NO: 231
BI-655064 LCVR=SEQ ID NO: 232
BI-655064 HC=SEQ ID NO: 236
BI-655064 LC=SEQ ID NO: 237

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 160/161. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 154, an HCDR2 comprising the amino acid sequence of SEQ ID NO: 155, an HCDR3 comprising the amino acid sequence of SEQ ID NO: 156, an LCDR1 comprising the amino acid sequence of SEQ ID NO: 157, an LCDR2 comprising the amino acid sequence of SEQ ID NO: 158, and an LCDR3 comprising the amino acid sequence of SEQ ID NO: 159.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 160; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 161. In some embodiments, the anti-CD40 antibody comprises an HCVR comprising or consisting of the amino acid sequence of SEQ ID NO: 160. In some embodiments, the anti-CD40 antibody comprises an LCVR comprising or consisting of the amino acid sequence of SEQ ID NO: 161.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 162; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 163. In some embodiments, the anti-CD40 antibody comprises a heavy chain comprising or consisting of the amino acid sequence of SEQ ID NO: 162. In some embodiments, the anti-CD40 antibody comprises a light chain comprising or consisting of the amino acid sequence of SEQ ID NO: 163. In some embodiments, the anti-CD40 antibody is bleselumab.
Bleselumab HCDR1=SEQ ID NO: 154
Bleselumab HCDR2=SEQ ID NO: 155
Bleselumab HCDR3=SEQ ID NO: 156
Bleselumab LCDR1=SEQ ID NO: 157
Bleselumab LCDR2=DAS (SEQ ID NO: 158)
Bleselumab LCDR3=SEQ ID NO: 159
Bleselumab HCVR=SEQ ID NO: 160
Bleselumab LCVR=SEQ ID NO: 161
Bleselumab HC=SEQ ID NO: 162
Bleselumab LC=SEQ ID NO: 163

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 170/171. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 164, an HCDR2 comprising the amino acid sequence of SEQ ID NO: 165, an HCDR3 comprising the amino acid sequence of SEQ ID NO: 166, an LCDR1 comprising the amino acid sequence of SEQ ID NO: 167, an LCDR2 comprising the amino acid sequence of SEQ ID NO: 168, and an LCDR3 comprising the amino acid sequence of SEQ ID NO: 169.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 170; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 171. In some embodiments, the anti-CD40 antibody comprises an HCVR comprising or consisting of the amino acid sequence of SEQ ID NO: 170. In some embodiments, the anti-CD40 antibody comprises an LCVR comprising or consisting of the amino acid sequence of SEQ ID NO: 171. In some embodiments, the anti-CD40 antibody is ch5D12.
ch5D12 HCDR1=SEQ ID NO: 164
ch5D12 HCDR2=SEQ ID NO: 165
ch5D12 HCDR3=SEQ ID NO: 166
ch5D12 LCDR1=SEQ ID NO: 167
ch5D12 LCDR2=SEQ ID NO: 168
ch5D12 LCDR3=SEQ ID NO: 169
ch5D12 HCVR=SEQ ID NO: 170
ch5D12 LCVR=SEQ ID NO: 171

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 141/142. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 135, an HCDR2 comprising the amino acid sequence of SEQ ID NO: 136, an HCDR3 comprising the amino acid sequence of SEQ ID NO: 137, an LCDR1 comprising the amino acid sequence of SEQ ID NO: 138, an LCDR2 comprising the amino acid sequence of SEQ ID NO: 139, and an LCDR3 comprising the amino acid sequence of SEQ ID NO: 140.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 141; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 142. In some embodiments, the anti-CD40 antibody comprises an HCVR comprising or consisting of the amino acid sequence of SEQ ID NO: 141. In some embodiments, the anti-CD40 antibody comprises an LCVR comprising or consisting of the amino acid sequence of SEQ ID NO: 142.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 172; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 144. In some embodiments, the anti-CD40 antibody comprises a heavy chain comprising or consisting of the amino acid sequence of SEQ ID NO: 172. In some embodiments, the anti-CD40 antibody comprises a light chain comprising or consisting of the amino acid sequence of SEQ ID NO: 144. In some embodiments, the anti-CD40 antibody is lucatumumab.
Lucatumumab (HCD122 or CHIR-12.12) HCDR1=SEQ ID NO: 135
Lucatumumab (HCD122 or CHIR-12.12) HCDR2=SEQ ID NO: 136
Lucatumumab (HCD122 or CHIR-12.12) HCDR3=SEQ ID NO: 137
Lucatumumab (HCD122 or CHIR-12.12) LCDR1=SEQ ID NO: 138
Lucatumumab (HCD122 or CHIR-12.12) LCDR2=SEQ ID NO: 139
Lucatumumab (HCD122 or CHIR-12.12) LCDR3=SEQ ID NO: 140
Lucatumumab (HCD122 or CHIR-12.12) HCVR=SEQ ID NO: 141
Lucatumumab (HCD122 or CHIR-12.12) LCVR=SEQ ID NO: 142
Lucatumumab (HCD122 or CHIR-12.12) HC=SEQ ID NO: 172
Lucatumumab (HCD122 or CHIR-12.12) LC=SEQ ID NO: 144

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 173/174. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 173; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 174. In some embodiments, the anti-CD40 antibody comprises an HCVR comprising or consisting of the amino acid sequence of SEQ ID NO: 173. In some embodiments, the anti-CD40 antibody comprises an LCVR comprising or consisting of the amino acid sequence of SEQ ID NO: 174.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 175; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 176. In some embodiments, the anti-CD40 antibody comprises a heavy chain comprising or consisting of the amino acid sequence of SEQ ID NO: 175. In some embodiments, the anti-CD40 antibody comprises a light chain comprising or consisting of the amino acid sequence of SEQ ID NO: 176. In some embodiments, the anti-CD40 antibody is CHIR-5.9.
CHIR-5.9 HCVR=SEQ ID NO: 173
CHIR-5.9 LCVR=SEQ ID NO: 174
CHIR-5.9 HC=SEQ ID NO: 175
CHIR-5.9 LC=SEQ ID NO: 176

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 188/189. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 164, an HCDR2 comprising the amino acid sequence of SEQ ID NO: 281, an HCDR3 comprising the amino acid sequence of SEQ ID NO: 166, an LCDR1 comprising the amino acid sequence of SEQ ID NO: 187, an LCDR2 comprising the amino acid sequence of SEQ ID NO: 168, and an LCDR3 comprising the amino acid sequence of SEQ ID NO: 169.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 188; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 189. In some embodiments, the anti-CD40 antibody comprises an HCVR comprising or consisting of the amino acid sequence of SEQ ID NO: 188. In some embodiments, the anti-CD40 antibody comprises an LCVR comprising or consisting of the amino acid sequence of SEQ ID NO: 189.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 190 or 191; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 192. In some embodiments, the anti-CD40 antibody comprises a heavy chain comprising or consisting of the amino acid sequence of SEQ ID NO: 190 or 191. In some embodiments, the anti-CD40 antibody comprises a light chain comprising or consisting of the amino acid sequence of SEQ ID NO: 192. In some embodiments, the anti-CD40 antibody is FFP104 (also known as PG102).
PG102//FFP104 HCDR1=SEQ ID NO: 164
PG102//FFP104 HCDR2=SEQ ID NO: 281
PG102//FFP104 HCDR3=SEQ ID NO: 166
PG102//FFP104 LCDR1=SEQ ID NO: 187
PG102//FFP104 LCDR2=SEQ ID NO: 168
PG102//FFP104 LCDR3=SEQ ID NO: 169
PG102//FFP104 HCVR=SEQ ID NO: 188
PG102//FFP104 LCVR=SEQ ID NO: 189
PG102//FFP104 HC=SEQ ID NO: 190 or 191
PG102//FFP104 LC=SEQ ID NO: 192

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 199/200. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 193, an HCDR2 comprising the amino acid sequence of SEQ ID NO: 194, an HCDR3 comprising the amino acid sequence of SEQ ID NO: 195, an LCDR1 comprising the amino acid sequence of SEQ ID NO: 196, an LCDR2 comprising the amino acid sequence of SEQ ID NO: 197, and an LCDR3 comprising the amino acid sequence of SEQ ID NO: 198.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 199; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 200. In some embodiments, the anti-CD40 antibody comprises an HCVR comprising or consisting of the amino acid sequence of SEQ ID NO: 199. In some embodiments, the anti-CD40 antibody comprises an LCVR comprising or consisting of the amino acid sequence of SEQ ID NO: 200.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 201; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 202. In some embodiments, the anti-CD40 antibody comprises a heavy chain comprising or consisting of the amino acid sequence of SEQ ID NO: 201. In some embodiments, the anti-CD40 antibody comprises a light chain comprising or consisting of the amino acid sequence of SEQ ID NO: 202. In some embodiments, the anti-CD40 antibody is BIIB063.
BIIB063 HCDR1=SEQ ID NO: 193
BIIB063 HCDR2=SEQ ID NO: 194
BIIB063 HCDR3=SEQ ID NO: 195
BIIB063 LCDR1=SEQ ID NO: 196
BIIB063 LCDR2=SEQ ID NO: 197
BIIB063 LCDR3=SEQ ID NO: 198
BIIB063 HCVR=SEQ ID NO: 199
BIIB063 LCVR=SEQ ID NO: 200
BIIB063 HC=SEQ ID NO: 201
BIIB063 LC=SEQ ID NO: 202

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3 amino acid sequences set contained within the HCVR amino acid sequence set forth in SEQ ID NO: 206. In some embodiments, the CDRs within the HCVR are identified according to the Kabat definition. In some embodiments, the CDRs within the HCVR are identified according to the Chothia definition. In some embodiments, the CDRs within the HCVR are identified according to the AbM definition. In some embodiments, the CDRs within the HCVR are identified according to the IMGT definition.

In some embodiments, the anti-CD40 antibody or antigen-binding fragment comprises an HCDR1 comprising the amino acid sequence of SEQ ID NO: 203, an HCDR2 comprising the amino acid sequence of SEQ ID NO: 204, and an HCDR3 comprising the amino acid sequence of SEQ ID NO: 205.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 206. In some embodiments, the anti-CD40 antibody comprises an HCVR comprising or consisting of the amino acid sequence of SEQ ID NO: 206. In some embodiments, the anti-CD40 antibody is V19.
V19 HCDR1=SEQ ID NO: 203
V19 HCDR2=SEQ ID NO: 204
V19 HCDR3=SEQ ID NO: 205
V19 HCVR=SEQ ID NO: 206

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3 amino acid sequences set contained within the HCVR amino acid sequence set forth in SEQ ID NO: 210. In some embodiments, the CDRs within the HCVR are identified according to the Kabat definition. In some embodiments, the CDRs within the HCVR are identified according to the Chothia definition. In some embodiments, the CDRs within the HCVR are identified according to the AbM definition. In some embodiments, the CDRs within the HCVR are identified according to the IMGT definition.

In some embodiments, the anti-CD40 antibody or antigen-binding fragment comprises an HCDR1 comprising the amino acid sequence of SEQ ID NO: 207, an HCDR2 comprising the amino acid sequence of SEQ ID NO: 208, and an HCDR3 comprising the amino acid sequence of SEQ ID NO: 209.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 210. In some embodiments, the anti-CD40 antibody comprises an HCVR comprising or consisting of the amino acid sequence of SEQ ID NO: 210. In some embodiments, the anti-CD40 antibody is V15.
V15 HCDR1=SEQ ID NO: 207
V15 HCDR2=SEQ ID NO: 208
V15 HCDR3=SEQ ID NO: 209
V15 HCVR=SEQ ID NO: 210

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 183/184, 183/212, 183/215, 183/217, 211/184, 211/212, 211/215, 211/217, 213/184, 213/212, 213/215, 213/217, 214/184, 214/212, 214/215, 214/217, 216/184, 216/212, 216/215, or 216/217. In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 183/184, 211/212, 213/212, 214/215, or 216/217. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 177, an HCDR2 comprising the amino acid sequence of SEQ ID NO: 178, an HCDR3 comprising the amino acid sequence of SEQ ID NO: 179, an LCDR1 comprising the amino acid sequence of SEQ ID NO: 180, an LCDR2 comprising the amino acid sequence of SEQ ID NO: 181, and an LCDR3 comprising the amino acid sequence of SEQ ID NO: 182.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 183, 211, 213, 214, or 216; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 184, 212, 215, or 217. In some embodiments, the anti-CD40 antibody comprises an HCVR comprising or consisting of the amino acid sequence of SEQ ID NO: 183, 211, 213, 214, or 216. In some embodiments, the anti-CD40 antibody comprises an LCVR comprising or consisting of the amino acid sequence of SEQ ID NO: 184, 212, 215, or 217. In some embodiments, the anti-CD40 antibody is h2C10 or a variant thereof.
2C10 HCDR1=SEQ ID NO: 177
2C10 HCDR2=SEQ ID NO: 178
2C10 HCDR3=SEQ ID NO: 179
2C10 LCDR1=SEQ ID NO: 180
2C10 LCDR2=SEQ ID NO: 181
2C10 LCDR3=SEQ ID NO: 182
2C10_h1 HCVR=SEQ ID NO: 211
2C10_h2 HCVR=SEQ ID NO: 213
2C10_h3 HCVR=SEQ ID NO: 183
2C10-11 LCVR=SEQ ID NO: 212
2C10-12 LCVR=SEQ ID NO: 184
2C10HP HCVR=SEQ ID NO: 183
2C10HB1 HCVR=SEQ ID NO: 214
2C10HB2 HCVR=SEQ ID NO: 216
2C10KP LCVR=SEQ ID NO: 184
2C10KB1 LCVR=SEQ ID NO: 215
2C10KB2 LCVR=SEQ ID NO: 217

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 151/268. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 145, an HCDR2 comprising the amino acid sequence of SEQ ID NO: 146, an HCDR3 comprising the amino acid sequence of SEQ ID NO: 147, an LCDR1 comprising the amino acid sequence of SEQ ID NO: 148, an LCDR2 comprising the amino acid sequence of SEQ ID NO: 149, and an LCDR3 comprising the amino acid sequence of SEQ ID NO: 150.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 151; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 268. In some embodiments, the anti-CD40 antibody comprises an HCVR comprising or consisting of the amino acid sequence of SEQ ID NO: 151. In some embodiments, the anti-CD40 antibody comprises an LCVR comprising or consisting of the amino acid sequence of SEQ ID NO: 268.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 218; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 153. In some embodiments, the anti-CD40 antibody comprises a heavy chain comprising or consisting of the amino acid sequence of SEQ ID NO: 218. In some embodiments, the anti-CD40 antibody comprises a light chain comprising or consisting of the amino acid sequence of SEQ ID NO: 153. In some embodiments, the anti-CD40 antibody is Ab101.
Ab101 HCDR1=SEQ ID NO: 145
Ab101 HCDR2=SEQ ID NO: 146
Ab101 HCDR3=SEQ ID NO: 147
Ab101 LCDR1=SEQ ID NO: 148
Ab101 LCDR2=SEQ ID NO: 149
Ab101 LCDR3=SEQ ID NO: 150
Ab101 HCVR=SEQ ID NO: 151
Ab101 LCVR=SEQ ID NO: 268
Ab101 HC=SEQ ID NO: 218
Ab101 LC=SEQ ID NO: 153

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 224/225. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 219, an HCDR2 comprising the amino acid sequence of SEQ ID NO: 220, an HCDR3 comprising the amino acid sequence of SEQ ID NO: 221, an LCDR1 comprising the amino acid sequence of SEQ ID NO: 222, an LCDR2 comprising the amino acid sequence of SEQ ID NO: 223, and an LCDR3 comprising the amino acid sequence of SEQ ID NO: 150.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 224; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 225. In some embodiments, the anti-CD40 antibody comprises an HCVR comprising or consisting of the amino acid sequence of SEQ ID NO: 224. In some embodiments, the anti-CD40 antibody comprises an LCVR comprising or consisting of the amino acid sequence of SEQ ID NO: 225.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 226, 227, 228, or 229; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 230. In some embodiments, the anti-CD40 antibody comprises a heavy chain comprising or consisting of the amino acid sequence of SEQ ID NO: 226, 227, 228, or 229. In some embodiments, the anti-CD40 antibody comprises a light chain comprising or consisting of the amino acid sequence of SEQ ID NO: 230. In some embodiments, the anti-CD40 antibody is Antibody A.
Antibody A HCDR1=SEQ ID NO: 219
Antibody A HCDR2=SEQ ID NO: 220
Antibody A HCDR3=SEQ ID NO: 221
Antibody A LCDR1=SEQ ID NO: 222
Antibody A LCDR2=SEQ ID NO: 223
Antibody A LCDR3=SEQ ID NO: 150
Antibody A HCVR=SEQ ID NO: 224
Antibody A LCVR=SEQ ID NO: 225
Antibody A HC=SEQ ID NO: 226 or 227 or 228 or 229
Antibody A LC=SEQ ID NO: 230

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 231/232. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 219, an HCDR2 comprising the amino acid sequence of SEQ ID NO: 220, an HCDR3 comprising the amino acid sequence of SEQ ID NO: 221, an LCDR1 comprising the amino acid sequence of SEQ ID NO: 222, an LCDR2 comprising the amino acid sequence of SEQ ID NO: 223, and an LCDR3 comprising the amino acid sequence of SEQ ID NO: 150.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 231; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 232. In some embodiments, the anti-CD40 antibody comprises an HCVR comprising or consisting of the amino acid sequence of SEQ ID NO: 231. In some embodiments, the anti-CD40 antibody comprises an LCVR comprising or consisting of the amino acid sequence of SEQ ID NO: 232.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 233, 234, 235, or 236; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 237. In some embodiments, the anti-CD40 antibody comprises a heavy chain comprising or consisting of the amino acid sequence of SEQ ID NO: 233, 234, 235, or 236. In some embodiments, the anti-CD40 antibody comprises a light chain comprising or consisting of the amino acid sequence of SEQ ID NO: 237. In some embodiments, the anti-CD40 antibody is Antibody B.
Antibody B HCDR1=SEQ ID NO: 219
Antibody B HCDR2=SEQ ID NO: 220
Antibody B HCDR3=SEQ ID NO: 221
Antibody B LCDR1=SEQ ID NO: 222
Antibody B LCDR2=SEQ ID NO: 223
Antibody B LCDR3=SEQ ID NO: 150
Antibody B HCVR=SEQ ID NO: 231
Antibody B LCVR=SEQ ID NO: 232
Antibody B HC=SEQ ID NO: 233 or 234 or 235 or 236
Antibody B LC=SEQ ID NO: 237

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 244/245. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 238, an HCDR2 comprising the amino acid sequence of SEQ ID NO: 239, an HCDR3 comprising the amino acid sequence of SEQ ID NO: 240, an LCDR1 comprising the amino acid sequence of SEQ ID NO: 241, an LCDR2 comprising the amino acid sequence of SEQ ID NO: 242, and an LCDR3 comprising the amino acid sequence of SEQ ID NO: 243.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 244; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 245. In some embodiments, the anti-CD40 antibody comprises an HCVR comprising or consisting of the amino acid sequence of SEQ ID NO: 244. In some embodiments, the anti-CD40 antibody comprises an LCVR comprising or consisting of the amino acid sequence of SEQ ID NO: 245.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 246, 247, 248, or 249; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 250. In some embodiments, the anti-CD40 antibody comprises a heavy chain comprising or consisting of the amino acid sequence of SEQ ID NO: 246, 247, 248, or 249. In some embodiments, the anti-CD40 antibody comprises a light chain comprising or consisting of the amino acid sequence of SEQ ID NO: 250. In some embodiments, the anti-CD40 antibody is Antibody C.
Antibody C HCDR1=SEQ ID NO: 238
Antibody C HCDR2=SEQ ID NO: 239
Antibody C HCDR3=SEQ ID NO: 240
Antibody C LCDR1=SEQ ID NO: 241
Antibody C LCDR2=SEQ ID NO: 242
Antibody C LCDR3=SEQ ID NO: 243
Antibody C HCVR=SEQ ID NO: 244
Antibody C LCVR=SEQ ID NO: 245
Antibody C HC=SEQ ID NO: 246 or 247 or 248 or 249
Antibody C LC=SEQ ID NO: 250

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 251/252. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 251; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 252. In some embodiments, the anti-CD40 antibody comprises an HCVR comprising or consisting of the amino acid sequence of SEQ ID NO: 251. In some embodiments, the anti-CD40 antibody comprises an LCVR comprising or consisting of the amino acid sequence of SEQ ID NO: 252. In some embodiments, the anti-CD40 antibody is G28.5.
G28.5 HCVR=SEQ ID NO: 251
G28.5 LCVR=SEQ ID NO: 252

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 259/260. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 253, an HCDR2 comprising the amino acid sequence of SEQ ID NO: 254, an HCDR3 comprising the amino acid sequence of SEQ ID NO: 255, an LCDR1 comprising the amino acid sequence of SEQ ID NO: 256, an LCDR2 comprising the amino acid sequence of SEQ ID NO: 257, and an LCDR3 comprising the amino acid sequence of SEQ ID NO: 258.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 259; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 260. In some embodiments, the anti-CD40 antibody comprises an HCVR comprising or consisting of the amino acid sequence of SEQ ID NO: 259. In some embodiments, the anti-CD40 antibody comprises an LCVR comprising or consisting of the amino acid sequence of SEQ ID NO: 260.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 261; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 262. In some embodiments, the anti-CD40 antibody comprises a heavy chain comprising or consisting of the amino acid sequence of SEQ ID NO: 261. In some embodiments, the anti-CD40 antibody comprises a light chain comprising or consisting of the amino acid sequence of SEQ ID NO: 262. In some embodiments, the anti-CD40 antibody is Y12XX-hz28.
Y12XX-hz28 (Vh-hzl4;Vk-hz2) HCDR1=SEQ ID NO: 253
Y12XX-hz28 (Vh-hzl4;Vk-hz2) HCDR2=SEQ ID NO: 254
Y12XX-hz28 (Vh-hzl4;Vk-hz2) HCDR3=SEQ ID NO: 255
Y12XX-hz28 (Vh-hzl4;Vk-hz2) LCDR1=SEQ ID NO: 256
Y12XX-hz28 (Vh-hzl4;Vk-hz2) LCDR2=SEQ ID NO: 257
Y12XX-hz28 (Vh-hzl4;Vk-hz2) LCDR3=SEQ ID NO: 258
Y12XX-hz28 (Vh-hzl4;Vk-hz2) HCVR=SEQ ID NO: 259
Y12XX-hz28 (Vh-hzl4;Vk-hz2) LCVR=SEQ ID NO: 260
Y12XX-hz28 (Vh-hzl4;Vk-hz2) HC=SEQ ID NO: 261
Y12XX-hz28 (Vh-hzl4;Vk-hz2) LC=SEQ ID NO: 262

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 264/265. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 253, an HCDR2 comprising the amino acid sequence of SEQ ID NO: 263, an HCDR3 comprising the amino acid sequence of SEQ ID NO: 255, an LCDR1 comprising the amino acid sequence of SEQ ID NO: 256, an LCDR2 comprising the amino acid sequence of SEQ ID NO: 257, and an LCDR3 comprising the amino acid sequence of SEQ ID NO: 258.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 264; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 265. In some embodiments, the anti-CD40 antibody comprises an HCVR comprising or consisting of the amino acid sequence of SEQ ID NO: 264. In some embodiments, the anti-CD40 antibody comprises an LCVR comprising or consisting of the amino acid sequence of SEQ ID NO: 265.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 266; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 267. In some embodiments, the anti-CD40 antibody comprises a heavy chain comprising or consisting of the amino acid sequence of SEQ ID NO: 266. In some embodiments, the anti-CD40 antibody comprises a light chain comprising or consisting of the amino acid sequence of SEQ ID NO: 267. In some embodiments, the anti-CD40 antibody is Y12XX-hz40.
Y12XX-hz40 (Vh-hzl2;Vk-hz3) HCDR1=SEQ ID NO: 253
Y12XX-hz40 (Vh-hzl2;Vk-hz3) HCDR2=SEQ ID NO: 263
Y12XX-hz40 (Vh-hzl2;Vk-hz3) HCDR3=SEQ ID NO: 255
Y12XX-hz40 (Vh-hzl2;Vk-hz3) LCDR1=SEQ ID NO: 256
Y12XX-hz40 (Vh-hzl2;Vk-hz3) LCDR2=SEQ ID NO: 257
Y12XX-hz40 (Vh-hzl2;Vk-hz3) LCDR3=SEQ ID NO: 258
Y12XX-hz40 (Vh-hzl2;Vk-hz3) HCVR=SEQ ID NO: 264
Y12XX-hz40 (Vh-hzl2;Vk-hz3) LCVR=SEQ ID NO: 265
Y12XX-hz40 (Vh-hzl2;Vk-hz3) HC=SEQ ID NO: 266
Y12XX-hz40 (Vh-hzl2;Vk-hz3) LC=SEQ ID NO: 267

In some embodiments, the present disclosure provides monospecific anti-CD40 antibodies or antigen-binding fragments thereof that specifically bind CD40 (e.g., human CD40). In some embodiments, the anti-CD40 antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 259/265. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40 antibody or antigen-binding fragment comprises an HCDR1 comprising the amino acid sequence of SEQ ID NO: 253, an HCDR2 comprising the amino acid sequence of SEQ ID NO: 254, an HCDR3 comprising the amino acid sequence of SEQ ID NO: 255, an LCDR1 comprising the amino acid sequence of SEQ ID NO: 256, an LCDR2 comprising the amino acid sequence of SEQ ID NO: 257, and an LCDR3 comprising the amino acid sequence of SEQ ID NO: 258.

In some embodiments, the anti-CD40 antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 259; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 265. In some embodiments, the anti-CD40 antibody comprises an HCVR comprising or consisting of the amino acid sequence of SEQ ID NO: 259. In some embodiments, the anti-CD40 antibody comprises an LCVR comprising or consisting of the amino acid sequence of SEQ ID NO: 265.

In some embodiments, the anti-CD40 antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 261; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 267. In some embodiments, the anti-CD40 antibody comprises a heavy chain comprising or consisting of the amino acid sequence of SEQ ID NO: 261. In some embodiments, the anti-CD40 antibody comprises a light chain comprising or consisting of the amino acid sequence of SEQ ID NO: 267. In some embodiments, the anti-CD40 antibody is Y12XX-hz42.
Y12XX-hz42 (Vh-hzl4;Vk-hz3) HCDR1=SEQ ID NO: 253
Y12XX-hz42 (Vh-hzl4;Vk-hz3) HCDR2=SEQ ID NO: 254
Y12XX-hz42 (Vh-hzl4;Vk-hz3) HCDR3=SEQ ID NO: 255
Y12XX-hz42 (Vh-hzl4;Vk-hz3) LCDR1=SEQ ID NO: 256
Y12XX-hz42 (Vh-hzl4;Vk-hz3) LCDR2=SEQ ID NO: 257
Y12XX-hz42 (Vh-hzl4;Vk-hz3) LCDR3=SEQ ID NO: 258
Y12XX-hz42 (Vh-hzl4;Vk-hz3) HCVR=SEQ ID NO: 259
Y12XX-hz42 (Vh-hzl4;Vk-hz3) LCVR=SEQ ID NO: 265
Y12XX-hz42 (Vh-hzl4;Vk-hz3) HC=SEQ ID NO: 261
Y12XX-hz42 (Vh-hzl4;Vk-hz3) LC=SEQ ID NO: 267

In some cases, the antibody comprises a human IgG heavy chain constant region. In some cases, the human IgG heavy chain constant region is isotype IgG1. In some cases, the human IgG heavy chain constant region is isotype IgG4.

### B. CD40×CD40 Bispecific Antigen-Binding Molecules

The present disclosure also provides multispecific antigen-binding molecules that specifically bind CD40. In some embodiments, the bispecific antigen-binding molecule comprises two CD40 binding aims comprising distinct HCVRs paired with a common LCVR. In some embodiments, the bispecific antigen-binding molecule comprises two CD40 binding arms comprising distinct HCVRs which are not paired with a common LCVR but instead are paired with distinct LCVRs which can comprise, e.g., without limitation any of the LCVRs, or combination thereof, disclosed herein. In some embodiments, the antigen-binding molecule is a bispecific antigen-binding molecule, e.g., bispecific antibody. Any of the anti-CD40 antibodies disclosed herein can be in bispecific configuration, including a second arm derived from any other anti-CD40 antibody disclosed herein. In some embodiments, the bispecific antigen-binding molecule comprises a first antigen-binding domain (D1) that binds a first epitope of CD40 (e.g., human CD40), and a second antigen-binding domain (D2) that binds a second epitope of CD40 (e.g., human CD40). In some embodiments, D1 and D2 do not compete with one another for binding to CD40 (e.g., human CD40). In some embodiments, D1 and D2 compete with one another for binding to CD40 (e.g., human CD40).

In some embodiments, the bispecific antigen-binding molecule comprises two different heavy chain immunoglobulin variable regions, wherein at least one heavy chain immunoglobulin variable region comprises an HCDR1-HCDR2-HCDR3 amino acid sequences set contained within an HCVR amino acid sequence selected from the group consisting of SEQ ID NOS: 2, 22, 32, 141, 151, 160, 170, 173, 183, 188, 199, 206, 210, 211, 213, 214, 216, 224, 231, 244, 251, 259, and 264. In some embodiments, the bispecific antigen-binding molecule comprises two different heavy chain immunoglobulin variable regions, wherein at least one heavy chain immunoglobulin variable region comprises an HCDR1-HCDR2-HCDR3 amino acid sequences set contained within an HCVR amino acid sequence selected from the group consisting of SEQ ID NOS: 2, 22, and 32. In some embodiments, each heavy chain immunoglobulin variable region comprises an HCDR1-HCDR2-HCDR3 amino acid sequences set contained within an HCVR amino acid sequence selected from the group consisting of SEQ ID NOS: 2, 22, 32, 141, 151, 160, 170, 173, 183, 188, 199, 206, 210, 211, 213, 214, 216, 224, 231, 244, 251, 259, and 264. In some embodiments, each heavy chain immunoglobulin variable region comprises an HCDR1-HCDR2-HCDR3 amino acid sequences set contained within an HCVR amino acid sequence selected from the group consisting of SEQ ID NOS: 2, 22, and 32. In some embodiments, the CDRs within the HCVR are identified according to the Kabat definition. In some embodiments, the CDRs are identified according to the Chothia definition. In some embodiments, the CDRs are identified according to the AbM definition. In some embodiments, the CDRs are identified according to the IMGT definition.

In some embodiments, at least one heavy chain immunoglobulin variable region comprises an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 2, 22, 32, 141, 151, 160, 170, 173, 183, 188, 199, 206, 210, 211, 213, 214, 216, 224, 231, 244, 251, 259, or 264. In some embodiments, at least one heavy chain immunoglobulin variable region comprises or consists of the amino acid sequence of SEQ ID NO: 2, 22, 32, 141, 151, 160, 170, 173, 183, 188, 199, 206, 210, 211, 213, 214, 216, 224, 231, 244, 251, 259, or 264.

In some embodiments, one or more of the heavy chain immunoglobulin variable regions comprises a set of HCDR sequences selected from the group consisting of: (a) an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; (b) an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28; and (c) an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38.

In some embodiments, the bispecific antigen-binding molecule comprises (i) an antigen-binding domain that comprises a heavy chain immunoglobulin variable region comprising an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; and (ii) an antigen-binding domain that comprises a heavy chain immunoglobulin variable region comprising an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28.

In some embodiments, the bispecific antigen-binding molecule comprises (i) an antigen-binding domain that comprises a heavy chain immunoglobulin variable region comprising an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; and (ii) an antigen-binding domain that comprises a heavy chain immunoglobulin variable region comprising an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38.

In some embodiments, the bispecific antigen-binding molecule comprises (i) an antigen-binding domain that comprises a heavy chain immunoglobulin variable region comprising an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28; and (ii) an antigen-binding domain that comprises a heavy chain immunoglobulin variable region comprising an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38.

In some embodiments, the bispecific antigen-binding molecule comprises a common light chain variable region. In some embodiments, the light chain variable region comprises an LCDR1-LCDR2-LCDR3 amino acid sequences set contained within the LCVR amino acid sequence of SEQ ID NO: 10. In some embodiments, the light chain variable region comprises an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some embodiments, the bispecific antigen-binding molecule comprises a D1 that binds a first epitope of human CD40, wherein the D1 domain comprises a heavy chain immunoglobulin chain comprising: (a) an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8; or (b) an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28.

In some embodiments, the D1 domain comprises an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8. In some embodiments, the D1 domain comprises an HCDR1 consisting of the amino acid sequence of SEQ ID NO: 4, an HCDR2 consisting of the amino acid sequence of SEQ ID NO: 6, and an HCDR3 consisting of the amino acid sequence of SEQ ID NO: 8. In some embodiments, the D1 domain comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 2. In some embodiments, the D1 domain comprises an HCVR comprising or consisting of the amino acid sequence of SEQ ID NO: 2

In some embodiments, the D1 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 46. In some embodiments, the D1 domain comprises a heavy chain comprising or consisting of the amino acid sequence of SEQ ID NO: 46.

In some embodiments, the D1 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 52. In some embodiments, the D1 domain comprises a heavy chain comprising or consisting of the amino acid sequence of SEQ ID NO: 52.

In some embodiments, the D1 domain further comprises a light chain immunoglobulin chain comprising an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some embodiments, the D1 domain comprises a LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the D1 domain comprises an LCVR comprising the amino acid sequence of SEQ ID NO: 10.

In some embodiments, the D1 domain comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the D1 domain comprises a light chain comprising the amino acid sequence of SEQ ID NO: 20.

In some embodiments, the D1 domain comprises an HCDR1 comprising the amino acid sequence of SEQ ID NO: 24, an HCDR2 comprising the amino acid sequence of SEQ ID NO: 26, and an HCDR3 comprising the amino acid sequence of SEQ ID NO: 28. In some embodiments, the D1 domain comprises an HCDR1 consisting of the amino acid sequence of SEQ ID NO: 24, an HCDR2 consisting of the amino acid sequence of SEQ ID NO: 26, and an HCDR3 consisting of the amino acid sequence of SEQ ID NO: 28. In some embodiments, the D1 domain comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 22. In some embodiments, the D1 domain comprises an HCVR comprising or consisting of the amino acid sequence of SEQ ID NO: 22.

In some embodiments, the D1 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 42. In some embodiments, the D1 domain comprises a heavy chain comprising or consisting of the amino acid sequence of SEQ ID NO: 42.

In some embodiments, the D1 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 48. In some embodiments, the D1 domain comprises a heavy chain comprising or consisting of the amino acid sequence of SEQ ID NO: 48.

In some embodiments, the D1 domain further comprises a light chain immunoglobulin chain comprising an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some embodiments, the D1 domain comprises a LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the D1 domain comprises an LCVR comprising the amino acid sequence of SEQ ID NO: 10.

In some embodiments, the D1 domain comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the D1 domain comprises a light chain comprising the amino acid sequence of SEQ ID NO: 20.

In some embodiments, the bispecific antigen-binding molecule comprises a D2 that binds a second epitope of human CD40, wherein the D2 domain comprises a heavy chain immunoglobulin chain comprising an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38. In some embodiments, the D2 domain comprises an HCDR1 consisting of the amino acid sequence of SEQ ID NO: 34, an HCDR2 consisting of the amino acid sequence of SEQ ID NO: 36, and an HCDR3 consisting of the amino acid sequence of SEQ ID NO: 38. In some embodiments, the D2 domain comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 32. In some embodiments, the D2 domain comprises an HCVR comprising or consisting of the amino acid sequence of SEQ ID NO: 32.

In some embodiments, the D2 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 44. In some embodiments, the D2 domain comprises a heavy chain comprising or consisting of the amino acid sequence of SEQ ID NO: 44.

In some embodiments, the D2 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 50. In some embodiments, the D2 domain comprises a heavy chain comprising or consisting of the amino acid sequence of SEQ ID NO: 50.

In some embodiments, the D2 domain further comprises a light chain immunoglobulin chain comprising an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16.

In some embodiments, the D2 domain comprises an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the D2 domain comprises an LCVR comprising the amino acid sequence of SEQ ID NO: 10.

In some embodiments, the D2 domain comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the D2 domain comprises a light chain comprising the amino acid sequence of SEQ ID NO: 20.

In some embodiments, the bispecific antigen-binding molecule comprises a D1 domain that binds a first epitope of human CD40 and a D2 domain that binds a second epitope of human CD40, wherein the D1 domain comprises a heavy chain immunoglobulin chain comprising an HCDR1 comprising the amino acid sequence of SEQ ID NO: 34, an HCDR2 comprising the amino acid sequence of SEQ ID NO: 36, and an HCDR3 comprising the amino acid sequence of SEQ ID NO: 38. In some embodiments, the D1 domain comprises an HCDR1 consisting of the amino acid sequence of SEQ ID NO: 34, an HCDR2 consisting of the amino acid sequence of SEQ ID NO: 36, and an HCDR3 consisting of the amino acid sequence of SEQ ID NO: 38. In some embodiments, the D1 domain comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 32. In some embodiments, the D1 domain comprises an HCVR comprising or consisting of the amino acid sequence of SEQ ID NO: 32.

In some embodiments, the D1 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 283. In some embodiments, the D1 domain comprises a heavy chain comprising or consisting of the amino acid sequence of SEQ ID NO: 283.

In some embodiments, the D2 domain comprises a heavy chain immunoglobulin chain comprising an HCDR1 comprising the amino acid sequence of SEQ ID NO: 4, an HCDR2 comprising the amino acid sequence of SEQ ID NO: 6, and an HCDR3 comprising the amino acid sequence of SEQ ID NO: 8. In some embodiments, the D2 domain comprises an HCDR1 consisting of the amino acid sequence of SEQ ID NO: 4, an HCDR2 consisting of the amino acid sequence of SEQ ID NO: 6, and an HCDR3 consisting of the amino acid sequence of SEQ ID NO: 8. In some embodiments, the D2 domain comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 2. In some embodiments, the D2 domain comprises an HCVR comprising or consisting of the amino acid sequence of SEQ ID NO: 2.

In some embodiments, the D2 domain comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 285. In some embodiments, the D2 domain comprises a heavy chain comprising or consisting of the amino acid sequence of SEQ ID NO: 285.

In some embodiments, the D1 and/or D2 domain further comprises a light chain immunoglobulin chain comprising an LCDR1 comprising the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the D1 and/or D2 domain comprises a LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the D1 and/or D2 domain comprises an LCVR comprising the amino acid sequence of SEQ ID NO: 10. In some embodiments, the D1 and/or D2 domain comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the D1 and/or D2 domain comprises a light chain comprising the amino acid sequence of SEQ ID NO: 20.

In some embodiments, the bispecific antigen-binding molecule comprises: a D1 domain that binds a first epitope of human CD40, wherein the D1 domain comprises an immunoglobulin chain comprising an HCDR1 comprising or consisting of the amino acid sequence of SEQ ID NO: 4, an HCDR2 comprising or consisting of the amino acid sequence of SEQ ID NO: 6, an HCDR3 comprising or consisting of the amino acid sequence of SEQ ID NO: 8, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16; and a D2 domain that binds a second epitope of human CD40, wherein the D2 domain comprises an immunoglobulin chain comprising an HCDR1 comprising or consisting of the amino acid sequence of SEQ ID NO: 34, an HCDR2 comprising or consisting of the amino acid sequence of SEQ ID NO: 36, an HCDR3 comprising or consisting of the amino acid sequence of SEQ ID NO: 38, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16.

In some embodiments, the bispecific antigen-binding molecule comprises: a D1 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 2, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10; and a D2 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 32, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10.

In some embodiments, the bispecific antigen-binding molecule comprises: a D1 domain that comprises an HCVR comprising or consisting of the amino acid sequence of SEQ ID NO: 2 and an LCVR comprising or consisting of the amino acid sequence of SEQ ID NO: 10; and a D2 domain that comprises an HCVR comprising or consisting of the amino acid sequence of SEQ ID NO: 32 and an LCVR comprising or consisting of the amino acid sequence of SEQ ID NO: 10.

In some embodiments, the bispecific antigen-binding molecule comprises: a D1 domain that comprises a heavy chain comprising or consisting of the amino acid sequence of SEQ ID NO: 46 and a light chain comprising or consisting of the amino acid sequence of SEQ ID NO: 20; and a D2 domain that comprises a heavy chain comprising or consisting of the amino acid sequence of SEQ ID NO: 44 and a light chain comprising or consisting of the amino acid sequence of SEQ ID NO: 20.

In some embodiments, the bispecific antigen-binding molecule comprises: a D1 domain that comprises a heavy chain comprising or consisting of the amino acid sequence of SEQ ID NO: 52 and a light chain comprising or consisting of the amino acid sequence of SEQ ID NO: 20; and a D2 domain that comprises a heavy chain comprising or consisting of the amino acid sequence of SEQ ID NO: 50 and a light chain comprising or consisting of the amino acid sequence of SEQ ID NO: 20.

In some embodiments, the bispecific antigen-binding molecule comprises: a D1 domain that binds a first epitope of human CD40, wherein the D1 domain comprises an HCDR1 comprising the amino acid sequence of SEQ ID NO: 24, an HCDR2 comprising or consisting of the amino acid sequence of SEQ ID NO: 26, an HCDR3 comprising or consisting of the amino acid sequence of SEQ ID NO: 28, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16; and a D2 domain that binds a second epitope of human CD40, wherein the D2 domain comprises an HCDR1 comprising or consisting of the amino acid sequence of SEQ ID NO: 34, an HCDR2 comprising or consisting of the amino acid sequence of SEQ ID NO: 36, an HCDR3 comprising or consisting of the amino acid sequence of SEQ ID NO: 38, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16.

In some embodiments, the bispecific antigen-binding molecule comprises: a D1 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 22, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10; and a D2 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 32, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10.

In some embodiments, the bispecific antigen-binding molecule comprises: a D1 domain that comprises an HCVR comprising or consisting of the amino acid sequence of SEQ ID NO: 22 and an LCVR comprising or consisting of the amino acid sequence of SEQ ID NO: 10; and a D2 domain that comprises an HCVR comprising or consisting of the amino acid sequence of SEQ ID NO: 32 and an LCVR comprising or consisting of the amino acid sequence of SEQ ID NO: 10.

In some embodiments, the bispecific antigen-binding molecule comprises: a D1 domain that comprises a heavy chain comprising or consisting of the amino acid sequence of SEQ ID NO: 42 and a light chain comprising or consisting of the amino acid sequence of SEQ ID NO: 20; and a D2 domain that comprises a heavy chain comprising or consisting of the amino acid sequence of SEQ ID NO: 44 and a light chain comprising or consisting of the amino acid sequence of SEQ ID NO: 20.

In some embodiments, the bispecific antigen-binding molecule comprises: a D1 domain that comprises a heavy chain comprising or consisting of the amino acid sequence of SEQ ID NO: 48 and a light chain comprising or consisting of the amino acid sequence of SEQ ID NO: 20; and a D2 domain that comprises a heavy chain comprising or consisting of the amino acid sequence of SEQ ID NO: 50 and a light chain comprising or consisting of the amino acid sequence of SEQ ID NO: 20.

In some embodiments, the bispecific antigen-binding molecule comprises: a D1 domain that binds a first epitope of human CD40, wherein the D1 domain comprises an immunoglobulin chain comprising an HCDR1 comprising or consisting of the amino acid sequence of SEQ ID NO: 34, an HCDR2 comprising or consisting of the amino acid sequence of SEQ ID NO: 36, an HCDR3 comprising or consisting of the amino acid sequence of SEQ ID NO: 38, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16; and a D2 domain that binds a second epitope of human CD40, wherein the D2 domain comprises an immunoglobulin chain comprising an HCDR1 comprising or consisting of the amino acid sequence of SEQ ID NO: 4, an HCDR2 comprising or consisting of the amino acid sequence of SEQ ID NO: 6, an HCDR3 comprising or consisting of the amino acid sequence of SEQ ID NO: 8, an LCDR1 comprising or consisting of the amino acid sequence of SEQ ID NO: 12, an LCDR2 comprising or consisting of the amino acid sequence AAS (SEQ ID NO: 14), and an LCDR3 comprising or consisting of the amino acid sequence of SEQ ID NO: 16.

In some embodiments, the bispecific antigen-binding molecule comprises: a D1 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 32, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10; and a D2 domain that comprises an HCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 2, and an LCVR comprising an amino acid sequence that has at least 85% (e.g., at least 90%, at least 91%, at least 92%, at least 93%, at least 94%, at least 95%, at least 96%, at least 97%, at least 98%, or at least 99%) sequence identity to SEQ ID NO: 10.

In some embodiments, the bispecific antigen-binding molecule comprises: a D1 domain that comprises an HCVR comprising or consisting of the amino acid sequence of SEQ ID NO: 32 and an LCVR comprising or consisting of the amino acid sequence of SEQ ID NO: 10; and a D2 domain that comprises an HCVR comprising or consisting of the amino acid sequence of SEQ ID NO: 2 and an LCVR comprising or consisting of the amino acid sequence of SEQ ID NO: 10.

In some embodiments, the bispecific antigen-binding molecule comprises: a D1 domain that comprises a heavy chain comprising or consisting of the amino acid sequence of SEQ ID NO: 283 and a light chain comprising or consisting of the amino acid sequence of SEQ ID NO: 20; and a D2 domain that comprises a heavy chain comprising or consisting of the amino acid sequence of SEQ ID NO: 285 and a light chain comprising or consisting of the amino acid sequence of SEQ ID NO: 20.

The bispecific antigen-binding molecules disclosed herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4.

The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

In some embodiments, a bispecific antigen-binding molecules of the present disclosure comprises two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In some embodiments, the multimerizing domain is an Fc fragment or an amino acid sequence of from 1 to about 200 amino acids in length, containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

In some embodiments, the bispecific antigen-binding molecule is an anti-CD40×CD40 bispecific antibody that has at least 85% sequence identity, e.g., at least 90%, at least 91%, at least 92%, at least 93%, at least 94%, at least 95%, at least 96%, at least 97%, at least 98% or at least 99% sequence identity, to REGN16334. In some embodiments, the bispecific antigen-binding molecule is an anti-CD40×CD40 bispecific antibody having the amino acid sequences of REGN16334.

In some embodiments, the bispecific antigen-binding molecule is an anti-CD40×CD40 bispecific antibody that has at least 85% sequence identity, e.g., at least 90%, at least 91%, at least 92%, at least 93%, at least 94%, at least 95%, at least 96%, at least 97%, at least 98% or at least 99% sequence identity, to REGN16335. In some embodiments, the bispecific antigen-binding molecule is a bispecific antibody having the amino acid sequences of REGN16335.

In some embodiments, the bispecific antigen-binding molecule is an anti-CD40×CD40 bispecific antibody that has at least 85% sequence identity, e.g., at least 90%, at least 91%, at least 92%, at least 93%, at least 94%, at least 95%, at least 96%, at least 97%, at least 98% or at least 99% sequence identity, to REGN16431. In some embodiments, the bispecific antigen-binding molecule is an anti-CD40×CD40 bispecific antibody having the amino acid sequences of REGN16431.

In some embodiments, the bispecific antigen-binding molecule is an anti-CD40×CD40 bispecific antibody that has at least 85% sequence identity, e.g., at least 90%, at least 91%, at least 92%, at least 93%, at least 94%, at least 95%, at least 96%, at least 97%, at least 98% or at least 99% sequence identity, to REGN16432. In some embodiments, the bispecific antigen-binding molecule is an anti-CD40×CD40 bispecific antibody having the amino acid sequences of REGN16432.

In some embodiments, the bispecific antigen-binding molecule is a CD40×CD40 bispecific antibody that has at least 85% sequence identity, e.g., at least 90%, at least 91%, at least 92%, at least 93%, at least 94%, at least 95%, at least 96%, at least 97%, at least 98% or at least 99% sequence identity, to REGN20484. In some embodiments, the bispecific antigen-binding molecule is a CD40×CD40 bispecific antibody having the amino acid sequences of REGN20484.

### C. Polynucleotides, Vectors, and Host Cells

In another aspect, the present disclosure provides nucleic acid molecules comprising one or more polynucleotide sequences encoding the antigen-binding molecules disclosed herein, as well as vectors (e.g., expression vectors) encoding such polynucleotide sequences and host cells into which such vectors have been introduced.

Polynucleotides, as disclosed herein, may encode all or a portion of an antigen-binding molecule, antibody, or antigen-binding fragment as disclosed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR, respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as disclosed above or herein.

In some embodiments, the nucleic acid molecule comprises one or more polynucleotide sequences encoding an antigen-binding molecule disclosed in any of Tables 9-12. In some embodiments, the nucleic acid molecule comprises one or more polynucleotide sequences set forth in Table 46.

In some embodiments, the nucleic acid molecule comprises a polynucleotide sequence that encodes a CD40 HCVR comprising the HCDR1, HCDR2, and HCDR3 of SEQ ID NOS: 4, 6, and 8, respectively, of SEQ ID NOS: 24, 26, and 28, respectively, or of SEQ ID NOS: 34, 36, and 38, respectively. In some embodiments, the nucleic acid molecule comprises a polynucleotide sequence that encodes a CD40 HCVR comprising or consisting of the sequence of SEQ ID NO: 2, SEQ ID NO: 22, or SEQ ID NO: 32.

In some embodiments, the nucleic acid molecule comprises a polynucleotide sequence that encodes an LCVR comprising an LCDR1 comprising or consisting of the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the nucleic acid molecule comprises a polynucleotide sequence that encodes an LCVR comprising or consisting of the sequence of SEQ ID NO: 10.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding molecule that binds a first epitope of CD40, and a second nucleic acid molecule comprising a polynucleotide sequence encoding an HCVR and/or LCVR of a second antigen-binding molecule that binds a second epitope of CD40. In some embodiments, a composition comprises a first nucleic acid molecule comprising a polynucleotide sequence encoding an HCVR of a first antigen-binding molecule that binds a first epitope of CD40, a second nucleic acid molecule comprising a polynucleotide sequence encoding an LCVR of a first antigen-binding molecule that binds a first epitope of CD40, a third nucleic acid molecule comprising a polynucleotide sequence encoding an HCVR of a second antigen-binding molecule that binds a second epitope of CD40, and a fourth nucleic acid molecule comprising a polynucleotide sequence encoding an LCVR of a second antigen-binding molecule that binds a second epitope of CD40. In some embodiments, the HCVR sequences of the first and second antigen-binding molecules are selected from: a CD40 HCVR comprising the HCDR1, HCDR2, and HCDR3 of SEQ ID NOS: 4, 6, and 8, respectively, a CD40 HCVR comprising the HCDR1, HCDR2, and HCDR3 of SEQ ID NOS: 24, 26, and 28, respectively, and a CD40 HCVR comprising the HCDR1, HCDR2, and HCDR3 of SEQ ID NOS: 34, 36, and 38, respectively. In some embodiments, the HCVR sequences of the first and second antigen-binding molecules are selected from SEQ ID NO: 2, SEQ ID NO: 22, and SEQ ID NO: 32. In some embodiments, the LCVR sequences of first and second antigen-binding molecules each comprise an LCDR1 comprising or consisting of the amino acid sequence of SEQ ID NO: 12, an LCDR2 comprising the amino acid sequence AAS (SEQ ID NO: 14), and an LCDR3 comprising the amino acid sequence of SEQ ID NO: 16. In some embodiments, the LCVR sequences of first and second antigen-binding molecules each comprise the sequence of SEQ ID NO: 10.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

### D. Characterization of CD40×CD40 Bispecific Antigen-Binding Molecules

The present disclosure includes antibodies and antigen-binding fragments thereof that bind to human CD40 with high affinity, e.g., bispecific antigen-binding molecules that bind to two different epitopes of CD40. In some embodiments, the antibodies and antigen binding fragments thereof (e.g., bispecific antigen-binding molecules) bind to CD40 and inhibit CD40L-induced activation but do not have agonist activity and/or cytotoxic effector functions.

In some embodiments, the present disclosure includes bispecific antigen-binding molecules (e.g., bispecific antibodies as disclosed herein, such as a bispecific antibody having the amino acid sequences of REGN16334, REGN16335, REGN16431, or REGN16432) that bind human CD40 (e.g., at 25° C. or 37° C.) with a K_{D} of less than about 75 nM as measured by surface plasmon resonance, e.g., using an assay format as described in Example 2 herein. In certain embodiments, the antigen-binding molecules of the present disclosure bind human CD40 with a K_{D} of less than about 75 nM, less than about 70 nM, less than about 60 nM, less than about 50 nM, less than about 40 nM, less than about 30 nM, less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured by surface plasmon resonance, e.g., using an assay format as defined in Example 2 herein, or a substantially similar assay.

In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) having an improved half-life as compared to monospecific antibodies (e.g., parental CD40 antibodies). In some embodiments, the present disclosure includes bispecific antigen-binding molecules (e.g., bispecific antibodies as disclosed herein) that bind human CD40 with a dissociative half-life (t½) of greater than about 70 minutes as measured by surface plasmon resonance at 25° C., e.g., using an assay format as defined in Example 2 herein, or a substantially similar assay. In certain embodiments, the antibodies or antigen-binding fragments of the present disclosure bind human CD40 with a t, of greater than about 75 minutes, greater than about 80 minutes, greater than about 85 minutes, or greater than about 90 minutes, as measured by surface plasmon resonance at 25° C., e.g., using an assay format as defined in Example 2 herein, or a substantially similar assay.

In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) that inhibit binding of human CD40 (e.g., a monomeric form of hCD40) to human CD40L. In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) that inhibit binding of human CD40 (e.g., a dimeric form of hCD40) to human CD40L. In some embodiments, inhibition of CD40 binding to CD40L is measured using an ELISA-based blocking assay as described in Example 4 herein. In some embodiments, a bispecific antigen-binding molecule inhibits binding of human CD40 (e.g., hCD40 monomer) to human CD40L by at least 50%, at least 60%, at least 70%, at least 75%, at least 80%, at least 90%, or more, e.g., using an assay format as defined in Example 4 herein, or a substantially similar assay. In some embodiments, a bispecific antigen-binding molecule inhibits binding of human CD40 (e.g., hCD40 dimer) to human CD40L by at least 50%, at least 60%, at least 70%, at least 75%, at least 80%, at least 90%, or more, e.g., using an assay format as defined in Example 4 herein, or a substantially similar assay.

In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) that specifically interact (e.g., bind with) cells that express CD40. The extent to which an antigen-binding molecule binds cells that express CD40 can be assessed by flow cytometry, as illustrated in Example 5 below. For example, in some embodiments, the present disclosure provides anti-CD40×CD40 bispecific antibodies that specifically bind cells that express CD40 on the cell surface (e.g., primary human B cells, or a human B cell line such as Ramos 2G6.4C10). In some embodiments, the disclosure provides anti-CD40×CD40 bispecific antibodies that bind CD40-expressing cells or cell lines with an EC₅₀ value of about 10 nM or less, e.g., from about 0.5 nM to about 10 nM, e.g., an EC₅₀ value of about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM, as determined by flow cytometry as set forth in Example 5 or a substantially similar assay.

In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) that inhibit CD40L-induced activation. In some embodiments, CD40L-induced activation is measured using a reporter assay, such as a luciferase-based reporter assay that quantitatively assesses receptor activation in a CD40-expressing cell by measuring downstream gene expression. In some embodiments, the reporter assay is an assay described in Example 6 herein. In some embodiments, CD40L-induced activation is measured by a cytokine secretion assay (e.g., secretion of IL-6, IL-10, IL-23, or TNFα) in the presence of CD40L. In some embodiments, the cytokine secretion assay is performed in primary cells that express CD40 (e.g., human B cells). In some embodiments, the cytokine secretion assay is performed in a stable cell line, e.g., a cell line that expresses CD40 and a reporter gene such as luciferase. In some embodiments, the cytokine secretion assay is an assay described in Example 7 herein. In some embodiments, the anti-CD40×CD40 bispecific antibody inhibits CD40L-induced activation (e.g., reporter gene expression or cytokine secretion) by at least 50%, at least 55%, at least 60%, at least 65%, at least 70%, at least 75%, at least 80%, at least 85%, at least 90% or more, relative to a control or a reference value.

In some embodiments, the present disclosure includes bispecific antigen-binding molecules (e.g., bispecific antibodies as disclosed herein, such as a bispecific antibody having the amino acid sequences of REGN16334, REGN16335, REGN16431, REGN16432, or REGN20484) that do not significantly agonize CD40 in the absence of CD40L. As used herein, “do[es] not significantly agonize CD40 in the absence of CD40L” means that in the presence of the bispecific antigen-binding molecule and absence of CD40L, the level of activation of CD40 is less than 15%, e.g., less than 13%, less than 10%, less than 8%, or less than 6%, e.g., as measured by downstream gene expression or cytokine secretion. In some embodiments, agonism of CD40 is measured by a reporter assay, such as a luciferase-based reporter assay that quantitatively assesses receptor activation in a CD40-expressing cell by measuring downstream gene expression. In some embodiments, agonism of CD40 is measured by a cytokine secretion assay (e.g., secretion of IL-6, IL-10, IL-23, or TNFα) in the absence of CD40L. In some embodiments, the assay is an assay described in Example 6 or Example 8 herein.

### E. Other CD40 Inhibitors

Other CD40 inhibitors can be used in place of the CD40 antigen-binding molecules disclosed herein in any of the compositions, combinations, kits, or methods disclosed here. A CD40 inhibitor suitable for use in accordance with any of the methods described herein can comprise, without limitation, any of various CD40 antigen-binding molecules described herein, for example, anti-CD40 antibodies, or functional fragments thereof, including monospecific anti-CD40 antibodies, bispecific anti-CD40 antibodies, or multispecific anti-CD40 antibodies, or functional fragments thereof. In some embodiments, a CD40 inhibitor can comprise an CD40 antigen-binding molecule as disclosed herein. However, other CD40 inhibitors can also be used.

In some embodiments, a CD40 inhibitor may comprise daclizumab.

In some embodiments, a CD40 inhibitor may comprise, e.g., another CD40-CD40L inhibitor (e.g., an anti-CD40 antibody such as iscalimab [CFZ-533], belselumab [ASKP1240 or 341G2], BI 655064, ch5D12, abiprubart [KPL-404], lucatumumab [HCD122 or CHIR12.12], ravagalimab [ABBV-323], CHIR-5.9, 201A3, BIIB063, Ab101, Ab102, PG102, Antibody A, Antibody B, Antibody C, G28.5, h2C10, BMS3h-37, BMS3h-38, BMS3h-56, BMS3h-198, Y12XX-hz28 [Vh-hzl4;Vk-hz2], Y12XX-hz40 [Vh-hzl2;Vk-hz3], Y12XX-hz42 [Vh-hzl4;Vk-hz3], V19, 5C8, 6H4, FFP104, or teneliximab; or an anti-CD40L antibody or antigen-binding protein such as dapirolizumab pegol, dazodalibep [VIB4920], frexalimab [INX-021], letolizumab [BMS-986004], MR-1, ruplizumab [BG9588], tegoprubart [AT-1501], toralizumab [IDEC-131], or APB-A1).

In some embodiments, the present disclosure provides monospecific anti-CD40L antibodies or antigen-binding fragments thereof that specifically bind CD40L (e.g., human CD40L). In some embodiments, the anti-CD40L antibody or antigen-binding fragment thereof comprises the HCDR1-HCDR2-HCDR3-LCDR1-LCDR2-LCDR3 amino acid sequences set contained within the HCVR/LCVR amino acid sequence pair set forth in SEQ ID NOS: 276/277. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In some embodiments, the anti-CD40L antibody or antigen-binding fragment comprises an HCDR1 comprising the amino acid sequence of SEQ ID NO: 270, an HCDR2 comprising the amino acid sequence of SEQ ID NO: 271, an HCDR3 comprising the amino acid sequence of SEQ ID NO: 272, an LCDR1 comprising the amino acid sequence of SEQ ID NO: 273, an LCDR2 comprising the amino acid sequence of SEQ ID NO: 274, and an LCDR3 comprising the amino acid sequence of SEQ ID NO: 275.

In some embodiments, the anti-CD40L antibody comprises: an HCVR having at least 85%, at least 90%, at least 91%, at least 92%, at least 93%, at least 94%, at least 95%, at least 96%, at least 97%, at least 98%, or at least 99% sequence identity to SEQ ID NO: 276; and/or an LCVR having at least 85%, at least 90%, at least 91%, at least 92%, at least 93%, at least 94%, at least 95%, at least 96%, at least 97%, at least 98%, or at least 99% sequence identity to SEQ ID NO: 277. In some embodiments, the anti-CD40L antibody comprises an HCVR comprising or consisting of the amino acid sequence of SEQ ID NO: 276. In some embodiments, the anti-CD40L antibody comprises an LCVR comprising or consisting of the amino acid sequence of SEQ ID NO: 277.

In some embodiments, the anti-CD40L antibody comprises: a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 278; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 279. In some embodiments, the anti-CD40L antibody comprises a heavy chain comprising or consisting of the amino acid sequence of SEQ ID NO: 278. In some embodiments, the anti-CD40L antibody comprises a light chain comprising or consisting of the amino acid sequence of SEQ ID NO: 279. In some embodiments, the anti-CD40L antibody is tegoprubart.

The present disclosure also provides multispecific antigen-binding molecules that specifically bind CD40L. In some embodiments, the antigen-binding molecule is a bispecific antigen-binding molecule, e.g., bispecific antibody. Any of the anti-CD40L antibodies disclosed herein can be in bispecific configuration, including a second arm derived from any other anti-CD40L antibody disclosed herein. In some embodiments, the bispecific antigen-binding molecule comprises a first antigen-binding domain (D1) that binds a first epitope of CD40L (e.g., human CD40L), and a second antigen-binding domain (D2) that binds a second epitope of CD40L (e.g., human CD40L). In some embodiments, D1 and D2 do not compete with one another for binding to CD40L (e.g., human CD40L). In some embodiments, D1 and D2 compete with one another for binding to CD40L (e.g., human CD40L).

In some embodiments, the bispecific antigen-binding molecule comprises two different heavy chain immunoglobulin variable regions, wherein at least one heavy chain immunoglobulin variable region comprises an HCDR1-HCDR2-HCDR3 amino acid sequences set contained within an HCVR amino acid sequence set forth in SEQ ID NO: 276. In some embodiments, the CDRs within the HCVR are identified according to the Kabat definition. In some embodiments, the CDRs are identified according to the Chothia definition. In some embodiments, the CDRs are identified according to the AbM definition. In some embodiments, the CDRs are identified according to the IMGT definition.

In some embodiments, at least one heavy chain immunoglobulin variable region comprises an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 276. In some embodiments, at least one heavy chain immunoglobulin variable region comprises or consists of the amino acid sequence of SEQ ID NO: 276.

In some embodiments, the CD40L inhibitor may comprise a CD40L-binding protein comprising two Tn3 proteins fused to human serum albumin. In some embodiments, the CD40L binding protein comprises an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO: 280. In some embodiments, the CD40L binding protein comprises or consists of the amino acid sequence set forth in SEQ ID NO: 280. In some embodiments, the CD40L binding protein is dazodalibep.

In some embodiments, the CD40 inhibitor may comprise a peptide such as but not limited to KGYY₁₅ (see, e.g., Vaitaitis et al., A CD40-targeted peptide controls and reverses type 1 diabetes in NOD mice Diabetologia. 2014 November; 57(11):2366-73, herein incorporated by reference in its entirety for all purposes), KGYY₆ (Vaitaitis et al., A CD40 targeting peptide prevents severe symptoms in Experimental Autoimmune Encephalomyelitis J Neuroimmunol. 2019 Jul. 15; 332: 8-15, herein incorporated by reference in its entirety for all purposes) and CD40-CD40L blocking cyclic heptapetide (CLPTRHMAC (SEQ ID NO: 341)). In some embodiments, the CD40 inhibitor may comprise an oligonucleotide such as but not limited to NJA-730.

In some embodiments, the CD40 inhibitor may comprise a small molecule inhibitor (e.g., DRI-C21045, BI08898). In some embodiments, a small-molecule CD40 inhibitor may comprise any small-molecule as described, for example, in J. Chen, et al. Small-Molecule Inhibitors of the CD40-CD40L Costimulatory Protein-Protein Interaction J. Med. Chem., 60 (21) (2017), pp. 8906-8922; L. F. Silvian, et al. Small molecule inhibition of the TNF family cytokine CD40 ligand through a subunit fracture mechanism ACS Chem. Biol., 6 (6) (2011), pp. 636-647; G. M. Vaitaitis, et al.; E. Margolles-Clark, et al. Suramin inhibits the CD40-CD154 costimulatory interaction: a possible mechanism for immunosuppressive effects Biochem. Pharmacol., 77 (7) (2009), pp. 1236-1245; each of which is herein incorporated by reference in its entirety for all purposes.

The CD40 inhibitor can be used for inhibiting CD40L-induced activation of CD40, e.g., in or on a cell that expresses CD40 (e.g., a B cell, dendritic cell, monocyte, platelet, or macrophage). The CD40 inhibitor are able to suppress host B cell responses to new antigens. In AAV gene therapies, seronegative/naive subjects are dosed with AAV and develop antibody responses to the AAV capsid antigen. This antibody response prevents future re-dosing of AAV because the antibodies are neutralizing, and the antibody response is sustained for 10+ years. When AAV is co-administered with a CD40 inhibitor, the B cell response is suppressed and anti-AAV IgG and/or IgM responses are significantly suppressed. When a CD40 inhibitor is given during the period of AAV antigen exposure, the anti-AAV antibody response can be suppressed in animals. This allows re-dosing of any AAV gene therapy product.

In some embodiments, the CD40 inhibitor can prevent antibody formation against an immunogenic delivery vehicle described herein. As a non-limiting example, the CD40 inhibitor can prevent antibody formation against an AAV or portion thereof. The CD40 inhibitor can also prevent antibody formation against certain LNP components (e.g., anti-PEG IgG), which can improve efficacy of LNP redosing. The CD40 inhibitor can also prevent antibody formation against, e.g., a transgene product described herein.

In some embodiments, the CD40 inhibitor is an anti-CD40 antibody or a functional fragment thereof. In some embodiments, the anti-CD40 antibody is an anti-CD40×CD40 bispecific antibody or a functional fragment thereof, and both antigen-binding domains bind to CD40.

### F. Pharmaceutical Compositions

In another aspect, the present disclosure provides pharmaceutical compositions comprising the CD40 inhibitors (e.g., CD40 antigen-binding molecules, e.g., CD40×CD40 bispecific antigen-binding molecule (e.g., bispecific antibodies)) disclosed herein. For example, the present disclosure provides pharmaceutical compositions comprising the CD40 inhibitors (e.g., CD40 antigen-binding molecules, e.g., CD40×CD40 bispecific antigen-binding molecule (e.g., bispecific antibodies)) and/or immunogens (e.g., nucleic acid constructs, nuclease agents or nucleic acids encoding nuclease agents, or immunogenic delivery vehicles comprising such components) disclosed herein, optionally comprising a pharmaceutically acceptable carrier and/or excipient. Other pharmaceutical compositions comprise (i) a plasma cell depleting agent, (ii) a B cell depleting agent and/or an immunoglobulin depleting agent, and (iii) a pharmaceutically acceptable carrier and/or excipient. Other pharmaceutical compositions comprise (i) an immunogen (e.g., nucleic acid constructs, nuclease agents or nucleic acids encoding nuclease agents, or immunogenic delivery vehicles comprising such components), (ii) a plasma cell depleting agent, (iii) optionally, a B cell depleting agent and/or an immunoglobulin depleting agent, and (iv) a pharmaceutically acceptable carrier and/or excipient. The pharmaceutical compositions are formulated with one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

In some embodiments, the pharmaceutical composition comprises an injectable preparation, such as a dosage form for intravenous, subcutaneous, intracutaneous, and intramuscular injections, drip infusions, etc. These injectable preparations may be prepared by known methods. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antigen-binding molecule administered to a patient according to the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions as disclosed herein may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition, e.g., encapsulation in liposomes, microparticles, microcapsules, recombinant cells capable of expressing, e.g., recombinant viruses comprising any components of the compositions disclosed herein, and a soluble carrier system that takes advantage of receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, an antigen-binding molecule or a pharmaceutical composition comprising an antigen-binding molecule as disclosed herein is contained within a container. Thus, in another aspect, containers comprising an antigen-binding molecule and/or pharmaceutical composition as disclosed herein are provided. For example, in some embodiments, an antibody and/or pharmaceutical composition is contained within a container selected from the group consisting of a glass vial, a syringe, a pen delivery device, and an autoinjector.

In some embodiments, an antigen-binding molecule or pharmaceutical composition of the present disclosure is delivered, e.g., subcutaneously or intravenously, such as with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen delivery devices having applications, e.g., in subcutaneous delivery of a pharmaceutical composition of the present disclosure include, but are not limited to the SOLOSTAR™ pen (sanofi-aventis), the FLEXPEN™ (Novo Nordisk), the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antigen-binding molecule contained in the dosage form is about 5 to about 1000 mg, e.g., from about 5 to about 500 mg, from about 5 to about 100 mg, or from about 10 to about 250 mg.

In yet another aspect, the present disclosure includes compositions and therapeutic formulations comprising any of the exemplary antibodies and bispecific antigen-binding molecules described herein in combination with one or more additional therapeutic agents, and methods of treatment comprising administering such combinations to subjects in need thereof. In some embodiments, the additional therapeutic agent(s) is an immunomodulatory agent or anti-inflammatory agent. In some embodiments, the additional therapeutic agent(s) is immunosuppressive therapy. In some embodiments, the additional therapeutic agent(s) is a surgical procedure.

Exemplary additional therapeutic agents that may be combined with or administered in combination with a CD40 inhibitor (e.g., CD40 antigen-binding molecule) of the present disclosure include, e.g., another CD40-CD40L inhibitor (e.g., an anti-CD40 antibody such as iscalimab [CFZ-533], belselumab [ASKP1240 or 341G2], BI 655064, ch5D12, abiprubart [KPL-404], lucatumumab [HCD122 or CHIR12.12], ravagalimab [ABBV-323], CHIR-5.9, 201A3, BIIB063, Ab101, Ab102, PG102, Antibody A, Antibody B, Antibody C, G28.5, h2C10, BMS3h-37, BMS3h-38, BMS3h-56, BMS3h-198, Y12XX-hz28 [Vh-hzl4;Vk-hz2], Y12XX-hz40 [Vh-hzl2;Vk-hz3], Y12XX-hz42 [Vh-hzl4;Vk-hz3], V19, 5C8, 6H4, FFP104, or teneliximab; or an anti-CD40L antibody or antigen-binding protein such as dapirolizumab pegol, dazodalibep [VIB4920], frexalimab [INX-021], letolizumab [BMS-986004], MR-1, ruplizumab [BG9588], tegoprubart [AT-1501], toralizumab [IDEC-131], or APB-A1; or a CD40 inhibitor peptide [such as but not limited to KGYY₁₅, KGYY₆, or CD40-CD40L blocking cyclic heptapetide (CLPTRHMAC (SEQ ID NO: 341))]; or a CD40 inhibitor oligonucleotide [e.g., NJA-730]; or a small molecule inhibitor [e.g., DRI-C21045, BI08898 or any small-molecule as described, for example, in J. Chen, et al. Small-Molecule Inhibitors of the CD40-CD40L Costimulatory Protein-Protein Interaction J. Med. Chem., 60 (21) (2017), pp. 8906-8922; L. F. Silvian, et al. Small molecule inhibition of the TNF family cytokine CD40 ligand through a subunit fracture mechanism ACS Chem. Biol., 6 (6) (2011), pp. 636-647; G. M. Vaitaitis, et al.; E. Margolles-Clark, et al. Suramin inhibits the CD40-CD154 costimulatory interaction: a possible mechanism for immunosuppressive effects Biochem. Pharmacol., 77 (7) (2009), pp. 1236-1245; each of which is herein incorporated by reference in its entirety for all purposes]).

The additional therapeutically active component(s) may be administered just prior to, concurrent with, or shortly after the administration of a CD40 inhibitor (e.g., CD40 antigen-binding molecule) of the present disclosure. For the purposes of the present disclosure, such administration regimens are considered the administration of a CD40 inhibitor (e.g., CD40 antigen-binding molecule) “in combination with” an additional therapeutically active component.

The present disclosure includes pharmaceutical compositions in which a CD40 inhibitor (e.g., CD40 antigen-binding molecule) is co-formulated with one or more of the additional therapeutically active component(s) as described elsewhere herein.

### III. Plasma Cell Depleting Agents

In some embodiments, the compositions disclosed herein comprise or the methods disclosed herein include administering a therapeutically effective amount of a plasma cell depleting agent to a subject in need thereof with preexisting immunity against an immunogen (i.e., an immunogen to be administered to the subject, e.g., an immunogenic delivery vehicle such as, e.g., AAV). For example, the subject can have preexisting immunity to a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. As used herein, a “plasma cell depleting agent” refers to any molecule capable of specifically binding to a surface antigen on plasma cells and killing or depleting the plasma cells.

The plasma cell depleting agents can be administered to a subject in need thereof with preexisting immunity against an immunogen (i.e., an immunogen to be administered to the subject, e.g., an immunogenic delivery vehicle such as, e.g., AAV) either alone, or in combination with, a B cell depleting agent and/or an immunoglobulin depleting agent. For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. In various aspects, a plasma cell depleting agent may be combined or administered in combination with a B cell depleting agent, an immunoglobulin depleting agent, plasmapheresis, therapeutic plasma exchange, immunoadsorption, and/or an immunogen (e.g., nucleic acid construct, nuclease agent or CRISPR/Cas system, e.g., in an immunogenic delivery vehicle such as, e.g., AAV) disclosed herein to a subject with preexisting immunity against the immunogen (i.e., an immunogen to be administered to the subject, e.g., an immunogenic delivery vehicle such as, e.g., AAV). Suitable combinations comprising a plasma cell depleting agent are described in more detail elsewhere herein. In some embodiments, the plasma cell depleting agent of the present disclosure is capable of depleting plasma cells including, without limitation, long-lived plasma cells (LLPCs). In some embodiments, a plasma cell depleting agent is administered to a subject having a pre-existing immunity against an immunogen (e.g., an immunogenic delivery vehicle such as, e.g., AAV (e.g., AAV comprising a nucleic acid construct described herein)). In some embodiments, a plasma cell depleting agent is administered to a subject having a pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein. In some embodiments, a plasma cell depleting agent is administered to a subject having a pre-existing immunity against an AAV vector comprising a nucleic acid construct described herein.

As used herein, the term “immunogen” refers to any molecule that is capable of eliciting an immune response. This definition applies to the use of term “immunogen” anywhere throughout the present specification (e.g., in the context of CD40 inhibitors, plasma cell depleting agents, B cell depleting agents, or any other agents disclosed herein). Non-limiting examples of immunogens include immunogenic delivery vehicles such as viral vectors also termed herein “viral particles” (e.g., viral vectors derived from adeno-associated viruses (AAV), adenoviruses, retroviruses [e.g., lentiviruses], or oncolytic viruses [e.g., an adenovirus, a rhabdovirus, a herpes virus, a measles virus, a coxsackievirus, a poliovirus, a reovirus, a poxvirus, a parvovirus, Maraba virus, or Newcastle disease virus]) or portions thereof (e.g., capsid proteins), virus-like particles (VLPs), non-viral vectors (e.g., bacteriophages [such as lambda (X) bacteriophage, EMBL bacteriophage; bacterial vectors such as pBs, phagescript, PsiX174, pBluescript SK, pBs KS, pNH8a, pNH16a, pNH18a, pNH46a; pTrc99A, pKK223-3, pKK233-3, pDR540, and pRIT5]; eukaryotic vectors [such as pWLneo, pSV2cat, pOG44, PXR1, pSG, pSVK3, pBPV, pMSG and pSVL]; transposons [such as Sleeping Beauty transposon and PiggyBac transposon]; bacterial vectors, fungal vectors, and protozoal vectors), liposomes, lipid nanoparticles (LNPs), non-lipid nanoparticles, mammalian cells (e.g., allogeneic cells), and other carriers. Non-limiting examples of immunogens also include polypeptide molecules (e.g., proteins [e.g., therapeutic proteins or antibodies or fragments thereof], peptides), polynucleotide molecules (e.g., mRNAs, interfering nucleic acid molecules [RNAi, siRNA, shRNA], miRNAs, antisense oligonucleotides, ribozymes, aptamers, mixmers, or multimers), antigen-binding molecules fused to a payload, as well as naturally occurring or modified bacteria, fungi, protozoa, parasites, helminths, ectoparasites, or other microorganisms (including bacteria, fungi and other microorganisms found in microbiota). In some embodiments, the immunogen is an immunogenic delivery vehicle, a polypeptide, or a polynucleotide. In some embodiments, the immunogen is an immunogenic delivery vehicle (e.g., AAV) or a polypeptide or polynucleotide encoded by a nucleic acid construct or transgene within the immunogenic delivery vehicle. In some embodiments, the immunogen is an immunogenic delivery vehicle. In some embodiments, the immunogenic delivery vehicle is a viral vector. In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell. In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector. Glycans and lipids are further encompassed by the term immunogen as used herein. In some embodiments, an immunogen can be a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein.

In some embodiments, the plasma cell depleting agent can be an antibody, a small molecule compound, a nucleic acid, a polypeptide, or a functional fragment or variant thereof. Non-limiting examples of suitable plasma cell depleting agents include B cell maturation antigen (BCMA) targeting agents (described elsewhere herein), proteasome inhibitors [e.g., bortezomib (Velcade), carfilzomib (Kyprolis), ixazomib (Niniaro)], histone deacetylase inhibitors [e.g., panobinostat (Farydak)], B-cell activating factor (BAFF; also referred to as BLyS, TALL-1, or CD257) inhibitors (e.g., anti-BAFF antibodies such as belimumab, tabalumab, AMG570; or anti-BAFF receptor antibodies such as ianalumab), proliferation-inducing ligand (APRIL; also referred to as TNFSF13 or CD256) inhibitors (e.g., anti-APRIL antibodies such as BION-1301 or VIS624), G protein-coupled receptor, class C, group 5, member D (GPRC5D) inhibitors (e.g., anti-GPRC5D antibodies, anti-GPRC5D×CD3 bispecific antibodies such as talquetamab), Fc receptor homolog 5 (FcRH5; also referred to as FcRL5, IRTA2, or CD307) inhibitors (e.g., anti-FcRH5 antibodies, anti-FcRH5×CD3 bispecific antibodies such as Cevostamab), and cluster of differentiation 38 (CD38; also referred to as CADPR1 or ADPRC1) inhibitors (e.g., anti-CD38 antibodies).

In some embodiments, the plasma cell depleting agents used in the compositions and methods disclosed herein are BCMA targeting agents. As used herein, the term “BCMA targeting agent” refers to any molecule capable of binding specifically to BCMA that is expressed on the surface of a cell, e.g., a cell in a subject, thus targeting the cell for destruction. BCMA is expressed exclusively in B-cell lineage cells, particularly in the interfollicular region of the germinal center as well as on plasmablasts and differentiated plasma cells. BCMA is selectively induced during plasma cell differentiation and is required for optimal survival of long-lived plasma cells (LLPCs) in the bone marrow. Thus, a BCMA targeting agent binds to BCMA expressed on a plasma cell surface and mediates killing or depletion of cells that express BCMA (plasma cell depletion). In some embodiments, a BCMA targeting agent comprises a binding moiety that binds to plasma cell-surface-expressed BCMA (an antigen-binding moiety or antigen-binding fragment thereof) and a moiety that facilitates killing of said plasma cell. In some embodiments, the plasma cell-surface-expressed BCMA-binding moiety is an antibody or antigen-binding fragment thereof that binds specifically to BCMA. Such a BCMA-binding moiety can be linked (e.g., covalently bound) to a moiety that facilitates killing or destruction of the targeted plasma cell. The moiety that facilitates targeted killing of the bound plasma cell may be a molecule that directly kills the targeted cell (e.g., a cytotoxic agent) or may be a protein or fragment thereof that mediates killing of the targeted cell, e.g., by an immune cell, e.g., a T-cell. In the context of the present disclosure, the term “BCMA targeting agent” includes, but is not limited to, anti-BCMA antibodies that are conjugated to a therapeutic agent such as a cytotoxic drug (“BCMA ADC” or “anti-BCMA ADC,” e.g., Belantamab Mafodotin/GSK2857916, MED12228, HDP-101), chimeric antigenic receptors (CARs) that bind specifically to BCMA, (“BCMA CAR” or “anti-BCMA CAR”) and anti-BCMA×CD3 bispecific antibodies (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B).

In some embodiments, the BCMA targeting agent used in the context of the disclosed methods is an antibody-drug conjugate (ADC) comprising an anti-BCMA antibody and a cytotoxic drug. In some embodiments, the anti-BCMA antibody or antigen-binding fragment thereof and the cytotoxic agent are covalently attached via a linker. In general terms, the ADCs comprise: A-[L-P]_{y}, in which A is an antigen-binding molecule, e.g., an anti-BCMA antibody, or a fragment thereof, L is a linker, P is the payload or therapeutic moiety (e.g., cytotoxic agent), and y is an integer from 1 to 30. Examples of suitable cytotoxic agents and chemotherapeutic agents for forming ADCs are known in the art. Non-limiting examples of suitable cytotoxic agents that can be conjugated to anti-BCMA antibodies for use in the disclosed methods are auristatin such as monomethyl auristatin E (MMAE) or monomethyl auristatin F (MMAF), a tubulysin such as TUB-OH or TUB-OMOM, a tomaymycin derivative, a dolastatin derivative, or a maytansinoid such as DM1 or DM4. In some exemplary embodiments, an anti-BCMA ADC used in the present methods comprises the HCVR, LCVR, and/or CDR amino acid sequences of any of the anti-BCMA antigen-binding molecules disclosed herein.

Other anti-BCMA ADCs that can be used in the context of the methods of the present disclosure include, e.g., the ADCs referred to and known in the art as Belantamab Mafodotin (GSK2857916), AMG224, HDP-101, MED12228, and TBL-CLN1, or any of the anti-BCMA ADCs set forth, e.g., in International Patent Publications WO2011/108008, WO2014/089335, WO2017/093942, WO2017/143069, or WO2019/025983. The portions of the publications cited herein that identify anti-BCMA ADCs are hereby incorporated by reference.

In some embodiments, the BCMA targeting agent used in the context of the disclosed methods is a chimeric antigen receptor (CAR) that binds specifically to BCMA (“BCMA CAR”). Generally, a “chimeric antigen receptor” (CAR) exhibits a specific anti-target cellular immune activity and comprises a binding domain against a component present on the target cell, for example an antibody-based specificity for a desired antigen (e.g., BCMA on plasma cell), and a T cell receptor-activating intracellular domain. CARs typically comprise an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity. In certain embodiments, the BCMA CAR or antigen-binding fragment thereof comprises a HCVR, LCVR, and/or CDRs comprising the amino acid sequences of any of the antibodies set forth in US Patent Publication No. US 2020/0023010, which is hereby incorporated by reference in its entirety. In some exemplary embodiments, an anti-BCMA CAR used in the present methods comprises the HCVR, LCVR, and/or CDR amino acid sequences of any of the anti-BCMA antigen-binding molecules disclosed herein.

Other anti-BCMA CARs that can be used in the context of the methods of the present disclosure include, e.g., the CARs referred to and known in the art as bb2121, LCAR-B38M, and 4C8A, or any of the anti-BCMA CARs set forth, e.g., in WO 2015/052538, WO 2015/052536, WO 2016/094304, WO 2016/166630, WO 2016/151315, WO 2016/130598, WO 2017/183418, WO 2017/173256, WO 2017211900, WO 2017/130223, WO 2018/229492, WO 2018/085690, WO 2018/151836, WO 2018/028647, WO 2019/006072. The portions of the publications cited herein that identify anti-BCMA CARs are hereby incorporated by reference.

In some exemplary embodiments, the BCMA targeting agent used in the disclosed methods is a multispecific (e.g., bispecific) antibody, or a functional fragment thereof, that specifically binds B cell maturation antigen (BCMA) and CD3 (e.g., an anti-BCMA×CD3 bispecific antibody). The anti-BCMA×CD3 multispecific (e.g., bispecific) antibodies are useful for specific targeting and T-cell-mediated killing of cells that express BCMA. The terms “antibody,” “antigen-binding fragment,” “human antibody,” “recombinant antibody,” and other related terminology are defined above. In the context of anti-BCMA×CD3 antibodies and antigen-binding fragments thereof, the present disclosure includes the use of bispecific antibodies wherein one arm of an immunoglobulin is specific for BCMA or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. See, e.g., Kazane et al., J. Am. Chem. Soc., 2013, 135(1):340-46.

An anti-BCMA×CD3 bispecific antibody, or functional fragment thereof, may comprise any of various anti-BCMA×CD3 bispecific antibodies, or functional fragments thereof, disclosed herein, or any other such anti-BCMA×CD3 bispecific antibodies, or functional fragments thereof, known to persons of ordinary skill in the art (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B). In a specific embodiment, the anti-BCMA×CD3 bispecific antibody is REGN5458. In another specific embodiment, the anti-BCMA×CD3 bispecific antibody is REGN5459.

### A. BCMA×CD3 Antigen-Binding Molecules

In some embodiments, the present disclosure provides antigen-binding molecules including multispecific (e.g., bispecific) antibodies that specifically bind B cell maturation antigen (BCMA) and CD3 (e.g., an anti-BCMA×CD3 bispecific antibody). In some embodiments, the antigen-binding molecule is a multispecific (e.g., bispecific) antibody. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. In some embodiments, the multispecific antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second binding specificity. In some embodiments, the multispecific antibody contains an antigen-binding domain that is specific for BCMA and an antigen-binding domain that is specific for CD3.

The term “CD3,” as used herein, refers to an antigen which is expressed on T cells as part of the multimolecular T cell receptor (TCR) and which consists of a homodimer or heterodimer formed from the dimeric association of two of four receptor chains: CD3-epsilon, CD3-delta, CD3-zeta, and CD3-gamma (e.g., gamma/epsilon, delta/epsilon, and zeta/zeta). CD3 is required for T cell activation.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). Antibodies against CD3 have been shown to cluster CD3 on T cells, thereby causing T cell activation in a manner similar to the engagement of the TCR by peptide-loaded major histocompatibility complex (MHC) molecules. Thus, bispecific antigen-binding molecules that are capable of binding both CD3 and another antigen (e.g., CD20 or BCMA) would be useful in settings in which specific targeting and T cell-mediated killing of cells that express the non-CD3 antigen (e.g., CD20 or BCMA) is desired.

The antibodies and antigen-binding fragments of the present invention may bind soluble CD3 and/or cell surface-expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

As used herein, the expression “anti-CD3 antibody” includes both monovalent antibodies with a single specificity, as well as bispecific antibodies comprising one arm that binds CD3 and another arm that binds a different antigen, wherein the anti-CD3 arm comprises any of the HCVR/LCVR or CDR sequences, or functional fragments thereof, as set forth in Table 1 or Table 2 herein. Examples of anti-CD3 bispecific antibodies are described elsewhere herein. Exemplary anti-CD3 antibodies are also described in PCT International Application No. PCT/US2013/060511, which is herein incorporated by reference in its entirety.

The present disclosure includes bispecific antibodies and functional fragments thereof that bind human CD3 with high affinity. The present disclosure also includes bispecific antibodies and functional fragments thereof that bind human CD3 with medium or low affinity, depending on the therapeutic context and particular targeting properties that are desired. For example, in the context of a bispecific antigen-binding molecule, wherein one arm binds CD3 and a second arm binds another antigen (e.g., CD20 or BCMA), it may be desirable for the second arm to bind the non-CD3 (e.g., CD20 or BCMA) antigen with high affinity while the anti-CD3 arm binds CD3 with only moderate or low affinity. In this manner, preferential targeting of the antigen-binding molecule to cells expressing the non-CD3 (e.g., CD20 or BCMA) antigen may be achieved while avoiding general/untargeted CD3 binding and the consequent adverse side effects associated therewith.

In certain embodiments, the anti-CD3 antibodies induce T cell proliferation with an EC₅₀ value of less than about 0.33 pM, as measured by an in vitro T cell proliferation assay (e.g., assessing the proliferation of Jurkat cells or PBMCs in the presence of anti-CD3 antibodies). In certain embodiments, the anti-CD3 antibodies induce T cell proliferation (e.g., Jurkat cell proliferation and/or PBMC proliferation) with an EC₅₀ value of less than about 0.32 pM, less than about 0.31 pM, less than about 0.30 pM, less than about 0.28 pM, less than about 0.26 pM, less than about 0.24 pM, less than about 0.22 pM, or less than about 0.20 pM, as measured by an in vitro T cell proliferation assay.

In some embodiments, the anti-BCMA×CD3 bispecific antigen-binding molecule comprises a first antigen-binding domain (D1) that binds an epitope of BCMA (e.g., human BCMA), and a second antigen-binding domain (D2) that binds an epitope of CD3 (e.g., human CD3).

In some exemplary embodiments, the anti-BCMA×CD3 bispecific antibody, or antigen-binding fragment thereof, comprises a heavy chain variable region (HCVR), light chain variable region (LCVR), and/or complementarity determining regions (CDRs) comprising the amino acid sequences of any of the anti-BCMA×CD3 antibodies set forth in U.S. Pat. No. 11,384,153 and US 2020/0345843, which are hereby incorporated by reference in their entireties.

In some exemplary embodiments, an anti-BCMA×CD3 bispecific antibody or antigen-binding fragment thereof that can be used in the context of the present disclosure comprising a HCVR, a LCVR, and/or CDRs comprising the amino acid sequences of REGN5458 or REGN5459 as set forth in Table 1 below.

TABLE 1
Amino Acid Sequences (SEQ ID NOS) of Exemplary Anti-BCMAxCD3
Bispecific Antibodies.

| | Anti-BCMA | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN5458 | 287 | 289 | 291 | 293 | 311 | 313 | 315 | 317 | 303 | 305 | 307 | 309 |
| REGN5459 | 287 | 289 | 291 | 293 | 319 | 321 | 323 | 325 | 303 | 305 | 307 | 309 |

SEQ ID NO: 298-Anti-BCMA LCDR2 DNA Sequence
GCTGCATCC
SEQ ID NO: 299-Anti-BCMA LCDR2 Protein Sequence
AAS
SEQ ID NO: 306-Common LCDR2 DNA Sequence
GCTGCATCC
SEQ ID NO: 307-Common LCDR2 Protein sequence
AAS

In some embodiments, the anti-BCMA×CD3 bispecific antibody or antigen-binding fragment thereof that can be used in the present disclosure comprises: (a) a first antigen binding domain that binds specifically to BCMA; and (b) a second antigen-binding domain that binds specifically to CD3. In one embodiment, the anti-BCMA antigen-binding domain comprises the heavy chain complementarity determining regions (HCDRs) of a heavy chain variable region (HCVR) comprising the amino acid sequence of SEQ ID NO: 287 and the light chain complementarity determining regions (LCDRs) of a light chain variable region (LCVR) comprising the amino acid sequence of SEQ ID NO: 303. In one embodiment, the first antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 289; the HCDR2 comprises the amino acid sequence of SEQ ID NO: 291; the HCDR3 comprises the amino acid sequence of SEQ ID NO: 293; the LCDR1 comprises the amino acid sequence of SEQ ID NO: 305; the LCDR2 comprises the amino acid sequence of SEQ ID NO: 307; and the LCDR3 comprises the amino acid sequence of SEQ ID NO: 309. In one embodiment, the second antigen-binding domain comprises the heavy chain complementarity determining regions (HCDRs) of a heavy chain variable region (HCVR) comprising the amino acid sequence of SEQ ID NO: 311 or 319 and the light chain complementarity determining regions (LCDRs) of a light chain variable region (LCVR) comprising the amino acid sequence of SEQ ID NO: 303. In one embodiment, the second antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 313 or 321; the HCDR2 comprises the amino acid sequence of SEQ ID NO: 315 or 323; the HCDR3 comprises the amino acid sequence of SEQ ID NO: 317 or 325; the LCDR1 comprises the amino acid sequence of SEQ ID NO: 305; the LCDR2 comprises the amino acid sequence of SEQ ID NO: 307; and the LCDR3 comprises the amino acid sequence of SEQ ID NO: 309.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOS: 289, 291, and 293, and LCDR1, LCDR2, and LCDR3 domains, respectively, comprising the amino acid sequences of SEQ ID NOS: 305, 307, and 309; and (b) a second antigen binding domain that comprises HCDR1, HCDR2, and HCDR3 domains, respectively, comprising the amino acid sequences of SEQ ID NOS: 313, 315, and 317, and LCDR1, LCDR2, and LCDR3 domains, respectively, comprising the amino acid sequences of SEQ ID NOS: 305, 307, and 309. In one embodiment, the anti-BCMA×CD3 bispecific antibody or antigen-binding fragment thereof comprises: (a) a first antigen-binding domain that comprises a HCVR comprising the amino acid sequence of SEQ ID NO: 287 and a LCVR comprising the amino acid sequence of SEQ ID NO: 303; and (b) a second antigen-binding domain that comprises a HCVR comprising the amino acid sequence of SEQ ID NO: 311 and a LCVR comprising the amino acid sequence of SEQ ID NO: 303.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOS: 289, 291, and 293, and LCDR1, LCDR2, and LCDR3 domains, respectively, comprising the amino acid sequences of SEQ ID NOS: 305, 307, and 309; and (b) a second antigen binding domain that comprises HCDR1, HCDR2, and HCDR3 domains, respectively, comprising the amino acid sequences of SEQ ID NOS: 321, 323, and 325, and LCDR1, LCDR2, and LCDR3 domains, respectively, comprising the amino acid sequences of SEQ ID NOS: 305, 307, and 309. In one embodiment, the anti-BCMA/anti-CD3 bispecific antibody or antigen-binding fragment thereof comprises: (a) a first antigen-binding domain that comprises a HCVR comprising the amino acid sequence of SEQ ID NO: 287 and a LCVR comprising the amino acid sequence of SEQ ID NO: 303; and (b) a second antigen-binding domain that comprises a HCVR comprising the amino acid sequence of SEQ ID NO: 319 and a LCVR comprising the amino acid sequence of SEQ ID NO: 303.

Exemplary anti-BCMA×CD3 bispecific antibodies include the fully human bispecific antibodies known as REGN5458 and REGN5459. See, e.g., WO 2020/018820, US 2020/0024356, US 2022/0306758, and U.S. Pat. No. 11,384,153, each of which is herein incorporated by reference. According to certain exemplary embodiments, the methods of the present disclosure comprise the use of REGN5458 or REGN5459, or a bioequivalent thereof. As used herein, the term “bioequivalent” with respect to anti-BCMA×CD3 antibodies refers to antibodies or BCMA×CD3 binding proteins or fragments thereof that are pharmaceutical equivalents or pharmaceutical alternatives having a rate and/or extent of absorption that does not show a significant difference with that of a reference antibody (e.g., REGN5458 or REGN5459) when administered at the same molar dose under similar experimental conditions, either single dose or multiple dose; the term “bioequivalent” also includes antigen-binding proteins that bind to BCMA/CD3 and do not have clinically meaningful differences with the reference antibody (e.g., REGN5458 or REGN5459) with respect to safety, purity, and/or potency.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 287 and a LCVR having at least 90%, 91%, 92%, 93%, 94%, 95%, 96%, 97%, 98%, or 99% sequence identity to the amino acid sequence of SEQ ID NO: 303; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 311 and a LCVR having at least 90%, 91%, 92%, 93%, 94%, 95%, 96%, 97%, 98%, or 99% sequence identity to the amino acid sequence of SEQ ID NO: 303. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOS: 289, 291, and 293, respectively, and a HCVR having at least 90%, 91%, 92%, 93%, 94%, 95%, 96%, 97%, 98%, or 99% sequence identity to the amino acid sequence of SEQ ID NO: 287, and comprises three LCDRs (LCDR1, LCDR2 and LCDR3) comprising the amino acid sequences of SEQ ID NOS: 305, 307, and 309, respectively, and a LCVR having at least 90%, 91%, 92%, 93%, 94%, 95%, 96%, 97%, 98%, or 99% sequence identity to the amino acid sequence of SEQ ID NO: 303; and (b) a second antigen-binding domain that comprises three HCDRs (HCDR1, HCDR2 and HCDR3) comprising the amino acid sequences of SEQ ID NOS: 313, 315, and 317, respectively, and a HCVR having at least 90%, 91%, 92%, 93%, 94%, 95%, 96%, 97%, 98%, or 99% sequence identity to the amino acid sequence of SEQ ID NO: 311, and comprises three LCDRs (LCDR1, LCDR2 and LCDR3) comprising the amino acid sequences of SEQ ID NOS: 305, 307, and 309, respectively, and a LCVR having at least 90%, 91%, 92%, 93%, 94%, 95%, 96%, 97%, 98%, or 99% sequence identity to the amino acid sequence of SEQ ID NO: 303.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 287 and a LCVR having at least 90%, 91%, 92%, 93%, 94%, 95%, 96%, 97%, 98%, or 99% sequence identity to the amino acid sequence of SEQ ID NO: 303; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 319 and a LCVR having at least 90%, 91%, 92%, 93%, 94%, 95%, 96%, 97%, 98%, or 99% sequence identity to the amino acid sequence of SEQ ID NO: 303. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOS: 289, 291, and 293, respectively, and a HCVR having at least 90%, 91%, 92%, 93%, 94%, 95%, 96%, 97%, 98%, or 99% sequence identity to the amino acid sequence of SEQ ID NO: 287, and comprises three LCDRs (LCDR1, LCDR2 and LCDR3) comprising the amino acid sequences of SEQ ID NOS: 305, 307, and 309, respectively, and a LCVR having at least 90%, 91%, 92%, 93%, 94%, 95%, 96%, 97%, 98%, or 99% sequence identity to the amino acid sequence of SEQ ID NO: 303; and (b) a second antigen-binding domain that comprises three HCDRs (HCDR1, HCDR2 and HCDR3) comprising the amino acid sequences of SEQ ID NOS: 321, 323, and 325, respectively, and a HCVR having at least 90%, 91%, 92%, 93%, 94%, 95%, 96%, 97%, 98%, or 99% sequence identity to the amino acid sequence of SEQ ID NO: 319, and comprises three LCDRs (LCDR1, LCDR2 and LCDR3) comprising the amino acid sequences of SEQ ID NOS: 305, 307, and 309, respectively, and a LCVR having at least 90%, 91%, 92%, 93%, 94%, 95%, 96%, 97%, 98%, or 99% sequence identity to the amino acid sequence of SEQ ID NO: 303.

The present disclosure also includes variants of the anti-BCMA×CD3 antibodies described herein comprising any of the HCVR, LCVR, and/or CDR amino acid sequences disclosed herein with one or more conservative amino acid substitutions. For example, the present disclosure includes use of anti-BCMA×CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, the disclosure includes use of an anti-BCMA×CD3 antibody having HCVR, LCVR, and/or CDR amino acid sequences with 1, 2, 3, or 4 conservative amino acid substitutions relative to any of the HCVR, LCVR, and/or CDR amino acid sequences disclosed herein.

Other anti-BCMA×CD3 antibodies that can be used in the methods of the present disclosure include, e.g., the antibodies referred to and known in the art as pacanalotamab (AMG420), teclistamab (JNJ-64007957), AMG701, alnuctamab (CC-93269), EM801, EM901, elranatamab (PF-06863135), TNB383B (ABBV-383), and TNB384B, or any of the anti-BCMA×CD3 antibodies set forth, e.g., in WO 2013/072415, WO 2014/140248, WO 2014/122144, WO 2016/166629, WO 2016/079177, WO 2016/020332, WO 2017/031104, WO 2017/223111, WO 2017/134134, WO 2018/083204, or WO 2018/201051. The portions of the publications cited herein that identify anti-BCMA×CD3 antibodies are hereby incorporated by reference.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

The bispecific antigen-binding molecules disclosed herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In some embodiments, the antibody comprises a first heavy chain containing the HCVR of the first antigen-binding domain and a second heavy chain containing the HCVR of the second antigen-binding domain, wherein the first heavy chain comprises residues 1-450 of the amino acid sequence of SEQ ID NO: 326 and the second heavy chain comprises residues 1-449 of the amino acid sequence of SEQ ID NO: 327.

In some embodiments, the antibody comprises a common light chain containing the LCVR of the first and second antigen-binding domains, wherein the common light chain comprises the amino acid sequence of SEQ ID NO: 328.

In some embodiments, the anti-BCMA×CD3 bispecific antibody comprises a first heavy chain comprising the amino acid sequence of SEQ ID NO: 326, a second heavy chain comprising the amino acid sequence of SEQ ID NO: 327, and a common light chain comprising the amino acid sequence of SEQ ID NO: 328. In some cases, the mature form of the antibody may not include the C-terminal lysine residues of SEQ ID NOS: 326 and 327. Thus, in some cases the anti-BCMA binding arm comprises a heavy chain comprising residues 1-450 of SEQ ID NO: 326, and the anti-CD3 binding arm comprises a heavy chain comprising residues 1-449 of SEQ ID NO: 327.

The first antigen-binding domain and the second antigen-binding domain may be directly or indirectly connected to one another to form a bispecific antigen-binding molecule. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

In some embodiments, a bispecific antigen-binding molecule of the present disclosure comprises two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In some embodiments, the multimerizing domain is an Fc fragment or an amino acid sequence of from 1 to about 200 amino acids in length, containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

### B. Polynucleotides, Vectors, and Host Cells

In another aspect, the present disclosure provides nucleic acid molecules comprising one or more polynucleotide sequences encoding the antigen-binding molecules disclosed herein, as well as vectors (e.g., expression vectors) encoding such polynucleotide sequences and host cells into which such vectors have been introduced.

Polynucleotides, as disclosed herein, may encode all or a portion of an antigen-binding molecule, antibody, or antigen-binding fragment as disclosed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR, respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as disclosed above or herein.

In some embodiments, the nucleic acid molecule comprises one or more polynucleotide sequences encoding an antigen-binding molecule disclosed in Table 1.

In some embodiments, the nucleic acid molecule comprises a polynucleotide sequence that encodes an anti-BCMA HCVR comprising the HCDR1, HCDR2, and HCDR3 of SEQ ID NOS: 289, 291, and 293, respectively. In some embodiments, the nucleic acid molecule comprises a polynucleotide sequence that encodes an anti-BCMA HCVR comprising or consisting of the sequence of SEQ ID NO: 287. In some embodiments, the nucleic acid molecule comprises a polynucleotide sequence of SEQ ID NO: 286, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 286.

In some embodiments, the nucleic acid molecule comprises a polynucleotide sequence that encodes an anti-CD3 HCVR comprising the HCDR1, HCDR2, and HCDR3 of SEQ ID NOS: 313, 315, and 317, respectively; or of SEQ ID NOS: 321, 323, and 325, respectively. In some embodiments, the nucleic acid molecule comprises a polynucleotide sequence that encodes an anti-CD3 HCVR comprising or consisting of the sequence of SEQ ID NO: 311 or 319. In some embodiments, the nucleic acid molecule comprises a polynucleotide sequence of SEQ ID NO: 310 or 318, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 310 or 318.

In some embodiments, the nucleic acid molecule comprises a polynucleotide sequence that encodes an LCVR comprising an LCDR1 comprising or consisting of the amino acid sequence of SEQ ID NO: 305, an LCDR2 comprising the amino acid sequence AAS (SEQ ID NO: 307), and an LCDR3 comprising the amino acid sequence of SEQ ID NO: 309. In some embodiments, the nucleic acid molecule comprises a polynucleotide sequence that encodes an LCVR comprising or consisting of the sequence of SEQ ID NO: 303. In some embodiments, the nucleic acid molecule comprises the polynucleotide sequence of SEQ ID NO: 302, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 302.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds BCMA, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds BCMA, a second nucleic acid molecule comprising a polynucleotide sequence encoding an LCVR of a first antigen-binding domain that binds BCMA, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-BCMA HCVR comprises the HCDR1, HCDR2, and HCDR3 of SEQ ID NOS: 289, 291, and 293, respectively. In some embodiments, an anti-BCMA LCVR comprises LCDR1, LCDR2, and LCDR3 of SEQ ID NOS: 305, 307, and 309, respectively. In some embodiments, an anti-CD3 HCVR comprises the HCDR1, HCDR2, and HCDR3 of SEQ ID NOS: 313, 315, and 317, respectively; or the HCDR1, HCDR2, and HCDR3 of SEQ ID NOS: 321, 323, and 325, respectively. In some embodiments, an anti-CD3 LCVR comprises the LCDR1, LCDR2, and LCDR3 of SEQ ID NOS: 305, 307, and 309, respectively.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 287, a nucleotide sequence encoding the HCVR sequence of the anti-CD3 antigen-binding domain comprising SEQ ID NO: 311, and a nucleotide sequence encoding the LCVR sequence comprising SEQ ID NO: 303.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 287, a nucleotide sequence encoding the HCVR sequence of the anti-CD3 antigen-binding domain comprising SEQ ID NO: 319, and a nucleotide sequence encoding the LCVR sequence comprising SEQ ID NO: 303.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

### C. Characterization of BCMA×CD3 Bispecific Antigen-Binding Molecules

The present disclosure includes bispecific antigen-binding molecules (e.g., bispecific antibodies) and functional fragments thereof that bind to BCMA and CD3 (e.g., human BCMA and CD3) with high affinity.

In some embodiments, the present disclosure includes bispecific antigen-binding molecules (e.g., bispecific antibodies as disclosed herein) that bind BCMA and CD3 (e.g., at 25° C. or 37° C.) with a K_{D} of less than about 75 nM, e.g., as measured by surface plasmon resonance or a substantially similar assay. In certain embodiments, the antigen-binding molecules of the present disclosure bind human BCMA and CD3 with a K_{D} of less than about 75 nM, less than about 70 nM, less than about 60 nM, less than about 50 nM, less than about 40 nM, less than about 30 nM, less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured by surface plasmon resonance or a substantially similar assay.

In some embodiments, the present disclosure includes bispecific antigen-binding molecules (e.g., bispecific antibodies as disclosed herein) that specifically interact (e.g., bind with) cells that express BCMA and/or CD3. The extent to which an antigen-binding molecule binds cells that express BCMA and/or CD3 can be assessed by flow cytometry. For example, in some embodiments, the present disclosure provides anti-BCMA×CD3 bispecific antibodies that specifically bind cells that express BCMA and/or CD3 on the cell surface (e.g., human plasma cells and/or T cells). In some embodiments, the disclosure provides anti-BCMA×CD3 bispecific antibodies that bind BCMA and/or CD3-expressing cells or cell lines with an EC₅₀ value of about 10 nM or less, e.g., from about 0.5 nM to about 10 nM, e.g., an EC₅₀ value of about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM, e.g., as determined by flow cytometry or a substantially similar assay.

### IV. B Cell Depleting Agents

In some embodiments, the methods disclosed herein include administering a therapeutically effective amount of a B cell depleting agent to a subject with preexisting immunity against an immunogen (i.e., an immunogen to be administered to the subject, e.g., an immunogenic delivery vehicle such as, e.g., AAV) in need thereof (i.e., in combination with a plasma cell depleting agent). For example, the subject can have preexisting immunity to a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. As used herein, a “B cell depleting agent” refers to any molecule capable of specifically binding to a surface antigen on B cells and killing or depleting said B cell. Thus, in general, a B cell depleting agent can be any agent that binds to a B cell surface molecule. In some embodiments, the B cell depleting agent is capable of depleting B cells and plasma cells that express low levels of BCMA.

In various aspects, the present disclosure provides B cell depleting agents, which are administered to a subject in need thereof with preexisting immunity against an immunogen (i.e., an immunogen to be administered to the subject, e.g., an immunogenic delivery vehicle such as, e.g., AAV) in combination with a plasma cell depleting agent (e.g., an anti-BCMA×CD3 bispecific antibody, or a functional fragment thereof) and optionally in combination with an immunoglobulin depleting agent (e.g., an FcRn blocker such as, e.g., efgartigimod) and/or an immunogen. For example, the immunogen can be a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein such as, e.g., AAV (e.g., AAV comprising a nucleic acid construct described herein). In some embodiments, plasmapheresis, therapeutic plasma exchange, and/or immunoadsorption may be further combined with the administering of the B cell depleting agent and the plasma cell depleting agent.

In various aspects, the present disclosure provides B cell depleting agents combined with, or administered in combination with, plasma cell depleting agents (e.g., an anti-BCMA×CD3 bispecific antibody, or a functional fragment thereof) described herein to subjects with preexisting immunity against an immunogen (i.e., an immunogen to be administered to the subject, e.g., an immunogenic delivery vehicle such as, e.g., AAV). For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. In some embodiments, the B cell depleting agent may be administered to subjects with preexisting immunity against an immunogen (i.e., an immunogen to be administered to the subject, e.g., an immunogenic delivery vehicle such as, e.g., AAV) not only in combination with a plasma cell depleting agent but also in combination with an immunoglobulin depleting agent, plasmapheresis, therapeutic plasma exchange, or immunoadsorption, and/or an immunogen (e.g., nucleic acid construct, nuclease agent or CRISPR/Cas system, e.g., in an immunogenic delivery vehicle). Suitable combinations comprising a plasma cell depleting agent are described in more detail elsewhere herein.

In some embodiments, the B cell depleting agent is an agent that directly targets a B cell, e.g., an agent that binds to a B cell surface molecule. In some embodiments, the B cell depleting agent causes a reduction in the number of B cells in a subject (e.g., in a blood sample taken from the subject). In some embodiments, a B cell depleting agent may be useful for, e.g., eliminating non-plasma cell (e.g., non-long-lived plasma cell [LLPC] sources of immunogen (e.g., anti-AAV) nAbs. In some embodiments, the B cell depleting agent may capture a wider range of AAV-specific B cells and plasma cells that may not express high levels of BCMA (e.g., committed memory B cells and early plasmablasts).

In some embodiments, the B cell depleting agent comprises an anti-CD19 antibody (e.g., MEDI-551, tefasitamab, Inebilizumab, loncastuximab), an anti-CD20 antibody (e.g., rituximab, ocrelizumab, obinutuzumab, ublituximab, or ofatumumab), an anti-CD22 antibody (e.g., epratuzumab), an anti-CD79 antibody (e.g., polatuzumab), a bispecific anti-CD20×CD3 B cell depleting antibody (e.g. odronextamab, glofitamab, mosunetuzumab, epcoritamab), a bispecific anti-CD19×CD3 antibody (e.g., blinatumomab), a bispecific anti-CD22×CD3 antibody (e.g., inotuzumab), or functional fragments thereof, or any combination thereof.

In some embodiments, the B cell depleting agent is an agent that indirectly targets a B cell, e.g., by targeting a B cell survival factor. In some embodiments, the B cell depleting agent is a BLyS/BAFF inhibitor (e.g., belimumab, lanalumab, BR3-Fc, AMG-570, or AMG-623), an APRIL inhibitor (e.g., telitacicept, atacicept), or a BLyS receptor 3/BAFF receptor inhibitor (e.g., anti-BR3), or any combination thereof.

In some embodiments, the B cell depleting agent is selected from anti-CD19 antibodies, anti-CD20 antibodies, anti-CD22 antibodies, anti-CD79 antibodies, multispecific antibodies combining two or more of any of said antibody specificities, multispecific antibodies combining any of said antibody specificities with anti-CD3 antibodies, functional fragments of any of said antibodies, and any combinations thereof. In certain embodiments, the B cell depleting agent is an anti-CD20 antibody or a functional fragment thereof. In some embodiments, a multispecific anti-CD20 antibody or functional fragment thereof of the present disclosure targets CD20 and CD19. In some embodiments, the multispecific anti-CD20 antibody or functional fragment thereof is anti-CD19×CD20 bispecific antibody, or functional fragment thereof. In some embodiments, the B cell depleting agent comprises an anti-CD19 antibody and an anti-CD20 antibody.

In some embodiments, the B cell depleting agent comprises anti-CD19 and anti-CD20 antibodies (also referred to as “anti-CD19/CD20 antibodies” herein), or functional fragments thereof, disclosed herein.

In a specific embodiment, the B cell depleting agent comprises a bispecific antibody that specifically binds CD3 and CD19. Such antibodies may be referred to herein as, e.g., “anti-CD19/anti-CD3,” or “anti-CD19×CD3” or “CD19×CD3” bispecific antibodies, or other similar terminology.

In a specific embodiment, the B cell depleting agent comprises a bispecific antibody that specifically binds CD3 and CD20. Such antibodies may be referred to herein as, e.g., “anti-CD20/anti-CD3,” or “anti-CD20×CD3” or “CD20×CD3” bispecific antibodies, or other similar terminology.

As used herein, the expression “bispecific antibody” refers to an immunoglobulin protein comprising at least a first antigen-binding domain and a second antigen-binding domain. In some embodiments, the first antigen-binding domain specifically binds a first antigen (e.g., CD20), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B.” Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain can each be connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present disclosure, the multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present disclosure typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antibodies of the present disclosure, Fc domains may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US 2015/0266966, incorporated herein in its entirety.

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2][IgG1 C_{H}3]. These and other examples of chimeric Fc domains that can be included in any of the antigen-binding molecules of the present disclosure are described in US Patent Publication No. 2014/0243504, which is herein incorporated in its entirety. Chimeric Fc domains having these general structural arrangements, and variants thereof, can have altered Fc receptor binding, which in turn affects Fc effector function.

### A. CD20×CD3 Antigen-Binding Molecules

The term “CD20,” as used herein, refers to an antigen which is expressed on B cells and which consists of a non-glycosylated phosphoprotein expressed on the cell membranes of mature B cells. The human CD20 protein can have the amino acid sequence as in NCBI Reference Sequence NP_690605.1. As used herein, the expression “anti-CD20 antibody” includes monovalent antibodies with a single specificity, such as RITUXAN® (rituximab), as described in U.S. Pat. No. 7,879,984. Exemplary anti-CD20 antibodies are also described in U.S. Pat. No. 7,879,984 and PCT International Application No. PCT/US2013/060511, filed on Sep. 19, 2013, each incorporated by reference herein.

In some exemplary embodiments, the CD20 targeting agent used in the disclosed methods is a multispecific (e.g., bispecific) antibody, or a functional fragment thereof, that specifically binds CD20 and CD3 (e.g., an anti-CD20×CD3 bispecific antibody). The anti-CD20×CD3 multispecific (e.g., bispecific) antibodies are useful for specific targeting and T-cell-mediated killing of cells that express CD20. The terms “antibody,” “antigen-binding fragment,” “human antibody,” “recombinant antibody,” and other related terminology are defined above. In the context of anti-CD20×CD3 antibodies and antigen-binding fragments thereof, the present disclosure includes the use of bispecific antibodies wherein one arm of an immunoglobulin is specific for CD20 or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

The anti-CD20×CD3 bispecific antibodies are capable of simultaneously binding to human CD3 and human CD20. According to certain embodiments, the anti-CD20×CD3 bispecific antibodies specifically interact with cells that express CD3 and/or CD20. The extent to which the anti-CD20×CD3 bispecific antibodies binds cells that express CD3 and/or CD20 can be assessed by fluorescence activated cell sorting (FACS). In certain embodiments, the anti-CD20×CD3 bispecific antibodies specifically bind human T-cell lines which express CD3 (e.g., Jurkat), human B-cell lines which express CD20 (e.g., Raji), and primate T-cells (e.g., cynomolgus peripheral blood mononuclear cells [PBMCs]).

In some embodiments, the anti-CD20×CD3 bispecific antigen-binding molecule comprises a first antigen-binding domain (D1) that binds an epitope of CD20 (e.g., human CD20), and a second antigen-binding domain (D2) that binds an epitope of CD3 (e.g., human CD3).

According to certain exemplary embodiments of the present disclosure, the bispecific anti-CD20×CD3 antibody, or antigen-binding fragment thereof comprises heavy chain variable regions (A-HCVR and B-HCVR), light chain variable region (LCVR), and/or complementarity determining regions (CDRs) comprising any of the amino acid sequences of the bispecific anti-CD20×CD3 antibodies as set forth in US Patent Publication No. 20150266966, incorporated herein by reference in its entirety for all purposes. In certain exemplary embodiments, the bispecific anti-CD20×CD3 antibody or antigen-binding fragment thereof that can be used in the context of the methods of the present disclosure comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 329 and the light chain complementarity determining regions (LCDRs) of a light chain variable region (LCVR) comprising the amino acid sequence of SEQ ID NO: 330; and (b) a second antigen-binding arm comprising the heavy chain CDRs (B-HCDR1, B-HCDR2 and B-HCDR3) of a HCVR (B-HCVR) comprising the amino acid sequence of SEQ ID NO: 331 and the light chain CDRs of a LCVR comprising the amino acid sequence of SEQ ID NO: 330. According to certain embodiments, the A-HCDR1 comprises the amino acid sequence of SEQ ID NO: 332; the A-HCDR2 comprises the amino acid sequence of SEQ ID NO: 333; the A-HCDR3 comprises the amino acid sequence of SEQ ID NO: 334; the LCDR1 comprises the amino acid sequence of SEQ ID NO: 335; the LCDR2 comprises the amino acid sequence of SEQ ID NO: 336; the LCDR3 comprises the amino acid sequence of SEQ ID NO: 337; the B-HCDR1 comprises the amino acid sequence of SEQ ID NO: 338; the B-HCDR2 comprises the amino acid sequence of SEQ ID NO: 339; and the B-HCDR3 comprises the amino acid sequence of SEQ ID NO: 340. In yet other embodiments, the bispecific anti-CD20×CD3 antibody or antigen-binding fragment thereof comprises: (a) a first antigen-binding arm comprising a HCVR (A-HCVR) comprising SEQ ID NO: 329 and a LCVR comprising SEQ ID NO: 330; and (b) a second antigen-binding arm comprising a HCVR (B-HCVR) comprising SEQ ID NO: 331 and a LCVR comprising SEQ ID NO: 330.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 329, and three light chain CDRs (LCDR1, LCDR2 and LCDR3) contained within a light chain variable region (LCVR) comprising the amino acid sequence of SEQ ID NO: 330.

In some embodiments, the first antigen-binding domain that specifically binds to CD20 comprises a HCDR1 comprising the amino acid sequence of SEQ ID NO: 332, a HCDR2 comprising the amino acid sequence of SEQ ID NO: 333, a HCDR3 comprising the amino acid sequence of SEQ ID NO: 334, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 331, and three light chain CDRs (LCDR1, LCDR2 and LCDR3) contained within a light chain variable region (LCVR) comprising the amino acid sequence of SEQ ID NO: 330.

In some embodiments, the second antigen-binding domain that specifically binds to CD3 comprises a HCDR1 comprising the amino acid sequence of SEQ ID NO: 338, a HCDR2 comprising the amino acid sequence of SEQ ID NO: 339, a HCDR3 comprising the amino acid sequence of SEQ ID NO: 340, a LCDR1 comprising the amino acid sequence of SEQ ID NO: 335, a LCDR2 comprising the amino acid sequence of SEQ ID NO: 336, and a LCDR3 comprising the amino acid sequence of SEQ ID NO: 337.

In some embodiments, the anti-CD20×CD3 bispecific antibody or functional fragment thereof comprises: a first antigen-binding domain that comprises HCDR1, HCDR2, and HCDR3 comprising the amino acid sequences of SEQ ID NOS: 332, 333, and 334, respectively, and LCDR1, LCDR2, and LCDR3 comprising the amino acid sequences of SEQ ID NOS: 335, 336, and 337, respectively; and a second antigen-binding domain that comprises HCDR1, HCDR2, and HCDR3 comprising the amino acid sequences of SEQ ID NOS: 338, 339, and 340, respectively, and LCDR1, LCDR2, and LCDR3 comprising the amino acid sequences of SEQ ID NOS: 335, 336, and 337, respectively.

Other bispecific anti-CD20×CD3 antibodies that can be used in the context of the methods of the present disclosure include, e.g., any of the antibodies as set forth in US 2014/0088295, US 2015/0166661, and US 2017/0174781, each of which is herein incorporated by reference in its entirety for all purposes. An exemplary bispecific anti-CD20×CD3 antibody that can be used in the context of the methods of the present disclosure is the bispecific anti-CD20×CD3 antibody known as REGN1979 or bsAB1.

In some exemplary embodiments, an anti-CD20×CD3 bispecific antibody or antigen-binding fragment thereof that can be used in the context of the present disclosure comprising a HCVR, a LCVR, and/or CDRs comprising the amino acid sequences of REGN1979 as set forth in Table 2 below.

TABLE 2
Amino Acid Sequences (SEQ ID NOS) of Exemplary Anti-CD20xCD3
Bispecific Antibodies.

| | Anti-CD20 | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN1979 | 329 | 332 | 333 | 334 | 331 | 338 | 339 | 340 | 330 | 335 | 336 | 337 |

SEQ ID NO: 336-Common LCDR2 Protein Sequence
GAS

In some embodiments, the anti-CD20×CD3 bispecific antibody or antigen-binding fragment thereof that can be used in the present disclosure comprises: (a) a first antigen binding domain that binds specifically to CD20; and (b) a second antigen-binding domain that binds specifically to CD3. In one embodiment, the anti-CD20 antigen-binding domain comprises the heavy chain complementarity determining regions (A-HCDRs) of a heavy chain variable region (A-HCVR) comprising the amino acid sequence of SEQ ID NO: 329 and the light chain complementarity determining regions (LCDRs) of a light chain variable region (LCVR) comprising the amino acid sequence of SEQ ID NO: 330. In one embodiment, the first antigen-binding domain comprises three HCDRs (A-HCDR1, A-HCDR2 and A-HCDR3) and three LCDRs (LCDR1, LCDR2 and LCDR3), wherein the A-HCDR1 comprises the amino acid sequence of SEQ ID NO: 332; the A-HCDR2 comprises the amino acid sequence of SEQ ID NO: 333; the A-HCDR3 comprises the amino acid sequence of SEQ ID NO: 334; the LCDR1 comprises the amino acid sequence of SEQ ID NO: 335; the LCDR2 comprises the amino acid sequence of SEQ ID NO: 336; and the LCDR3 comprises the amino acid sequence of SEQ ID NO: 337.

In one embodiment, the second antigen-binding domain comprises the heavy chain complementarity determining regions (B-HCDRs) of a heavy chain variable region (B-HCVR) comprising the amino acid sequence of SEQ ID NO: 331 and the light chain complementarity determining regions (LCDRs) of a light chain variable region (LCVR) comprising the amino acid sequence of SEQ ID NO: 330. In one embodiment, the second antigen-binding domain comprises three HCDRs (B-HCDR1, B-HCDR2 and B-HCDR3) and three LCDRs (LCDR1, LCDR2 and LCDR3), wherein the B-HCDR1 comprises the amino acid sequence of SEQ ID NO: 338; the B-HCDR2 comprises the amino acid sequence of SEQ ID NO: 339; the B-HCDR3 comprises the amino acid sequence of SEQ ID NO: 340; the LCDR1 comprises the amino acid sequence of SEQ ID NO: 335; the LCDR2 comprises the amino acid sequence of SEQ ID NO: 336; and the LCDR3 comprises the amino acid sequence of SEQ ID NO: 337.

In one embodiment, the anti-CD20×CD3 bispecific antibody or antigen-binding fragment thereof comprises: (a) a first antigen-binding domain that comprises A-HCDR1, A-CDR2, and A-HCDR3 domains, respectively, comprising the amino acid sequences of SEQ ID NOs: 332, 333, and 334, and LCDR1, LCDR2, and LCDR3 domains, respectively, comprising the amino acid sequences of SEQ ID NOs: 335, 336, and 337; and (b) a second antigen binding domain that comprises B-HCDR1, B-HCDR2, and B-HCDR3 domains, respectively, comprising the amino acid sequences of SEQ ID NOs: 338, 339, and 340, and LCDR1, LCDR2, and LCDR3 domains, respectively, comprising the amino acid sequences of SEQ ID NOs: 335, 336, and 337. In one embodiment, the anti-CD20×CD3 bispecific antibody or antigen-binding fragment thereof comprises: (a) a first antigen-binding domain that comprises a A-HCVR comprising the amino acid sequence of SEQ ID NO: 329 and a LCVR comprising the amino acid sequence of SEQ ID NO: 330; and (b) a second antigen-binding domain that comprises a B-HCVR comprising the amino acid sequence of SEQ ID NO: 331 and a LCVR comprising the amino acid sequence of SEQ ID NO: 330.

Exemplary anti-CD20×CD3 bispecific antibodies include the fully human bispecific antibody known as REGN1979. See, e.g., US 2014/0088295, US 2015/0166661, and US 2017/0174781, each of which is herein incorporated by reference. According to certain exemplary embodiments, the methods of the present disclosure comprise the use of REGN1979, or a bioequivalent thereof. As used herein, the term “bioequivalent” with respect to anti-CD20×CD3 antibodies refers to antibodies or CD20×CD3 binding proteins or fragments thereof that are pharmaceutical equivalents or pharmaceutical alternatives having a rate and/or extent of absorption that does not show a significant difference with that of a reference antibody (e.g., REGN1979) when administered at the same molar dose under similar experimental conditions, either single dose or multiple dose; the term “bioequivalent” also includes antigen-binding proteins that bind to CD20/CD3 and do not have clinically meaningful differences with the reference antibody (e.g., REGN1979) with respect to safety, purity, and/or potency.

In some embodiments, the anti-CD20×CD3 bispecific antibody or antigen-binding fragment thereof comprises: (a) a first antigen-binding domain that comprises a A-HCVR having at least 90%, 91%, 92%, 93%, 94%, 95%, 96%, 97%, 98%, or 99% sequence identity to the amino acid sequence of SEQ ID NO: 329 and a LCVR having at least 90%, 91%, 92%, 93%, 94%, 95%, 96%, 97%, 98%, or 99% sequence identity to the amino acid sequence of SEQ ID NO: 330; and (b) a second antigen-binding domain that comprises a B-HCVR having at least 90%, 91%, 92%, 93%, 94%, 95%, 96%, 97%, 98%, or 99% sequence identity to the amino acid sequence of SEQ ID NO: 331 and a LCVR having at least 90%, 91%, 92%, 93%, 94%, 95%, 96%, 97%, 98%, or 99% sequence identity to the amino acid sequence of SEQ ID NO: 330. In some embodiments, the anti-CD20×CD3 bispecific antibody or antigen-binding fragment thereof comprises: (a) a first antigen-binding domain that comprises three HCDRs (A-HCDR1, A-HCDR2 and A-HCDR3) comprising the amino acid sequences of SEQ ID NOs: 332, 333, and 334, respectively, and an A-HCVR having at least 90%, 91%, 92%, 93%, 94%, 95%, 96%, 97%, 98%, or 99% sequence identity to the amino acid sequence of SEQ ID NO: 329, and comprises three LCDRs (LCDR1, LCDR2 and LCDR3) comprising the amino acid sequences of SEQ ID NOs: 335, 336, and 337, respectively, and a LCVR having at least 90%, 91%, 92%, 93%, 94%, 95%, 96%, 97%, 98%, or 99% sequence identity to the amino acid sequence of SEQ ID NO: 330; and (b) a second antigen-binding domain that comprises three HCDRs (B-HCDR1, B-HCDR2 and B-HCDR3) comprising the amino acid sequences of SEQ ID NOs: 338, 339, and 340, respectively, and a B-HCVR having at least 90%, 91%, 92%, 93%, 94%, 95%, 96%, 97%, 98%, or 99% sequence identity to the amino acid sequence of SEQ ID NO: 331, and comprises three LCDRs (LCDR1, LCDR2 and LCDR3) comprising the amino acid sequences of SEQ ID NOs: 335, 336, and 337, respectively, and a LCVR having at least 90%, 91%, 92%, 93%, 94%, 95%, 96%, 97%, 98%, or 99% sequence identity to the amino acid sequence of SEQ ID NO: 330.

The present disclosure also includes variants of the anti-CD20×CD3 antibodies described herein comprising any of the HCVR, LCVR, and/or CDR amino acid sequences disclosed herein with one or more conservative amino acid substitutions. For example, the present disclosure includes use of anti-CD20×CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, the disclosure includes use of an anti-CD20×CD3 antibody having HCVR, LCVR, and/or CDR amino acid sequences with 1, 2, 3, or 4 conservative amino acid substitutions relative to any of the HCVR, LCVR, and/or CDR amino acid sequences disclosed herein.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

In some embodiments, the anti-CD20×CD3 bispecific antibody or functional fragment thereof comprises a human IgG heavy chain constant region. In some embodiments, the human IgG heavy chain constant region is isotype IgG4 or IgG1. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

### B. Polynucleotides, Vectors, and Host Cells

In another aspect, the present disclosure provides nucleic acid molecules comprising one or more polynucleotide sequences encoding the antigen-binding molecules disclosed herein, as well as vectors (e.g., expression vectors) encoding such polynucleotide sequences and host cells into which such vectors have been introduced.

Polynucleotides, as disclosed herein, may encode all or a portion of an antigen-binding molecule, antibody, or antigen-binding fragment as disclosed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR, respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as disclosed above or herein.

In some embodiments, the nucleic acid molecule comprises one or more polynucleotide sequences encoding an antigen-binding molecule disclosed in Table 2.

In some embodiments, the nucleic acid molecule comprises a polynucleotide sequence that encodes an anti-CD20 HCVR comprising the HCDR1, HCDR2, and HCDR3 of SEQ ID NOs: 332, 333, and 334, respectively. In some embodiments, the nucleic acid molecule comprises a polynucleotide sequence that encodes an anti-CD20 HCVR comprising or consisting of the sequence of SEQ ID NO: 329.

In some embodiments, the nucleic acid molecule comprises a polynucleotide sequence that encodes an anti-CD3 HCVR comprising the HCDR1, HCDR2, and HCDR3 of SEQ ID NOs: 338, 339, and 340, respectively. In some embodiments, the nucleic acid molecule comprises a polynucleotide sequence that encodes an anti-CD3 HCVR comprising or consisting of the sequence of SEQ ID NO: 331.

In some embodiments, the nucleic acid molecule comprises a polynucleotide sequence that encodes an LCVR comprising the LCDR1, LCDR2, and LCDR3 of SEQ ID NOs: 335, 336, and 337, respectively. In some embodiments, the nucleic acid molecule comprises a polynucleotide sequence that encodes an LCVR comprising or consisting of the sequence of SEQ ID NO: 330.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds CD20, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds CD20, a second nucleic acid molecule comprising a polynucleotide sequence encoding an LCVR of a first antigen-binding domain that binds CD20, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-CD20 HCVR comprises the HCDR1, HCDR2, and HCDR3 of SEQ ID NOs: 332, 333, and 334, respectively. In some embodiments, an anti-CD20 LCVR comprises LCDR1, LCDR2, and LCDR3 of SEQ ID NOs: 335, 336, and 337, respectively. In some embodiments, an anti-CD3 HCVR comprises the HCDR1, HCDR2, and HCDR3 of SEQ ID NOs: 338, 339, and 340, respectively. In some embodiments, an anti-CD3 LCVR comprises the LCDR1, LCDR2, and LCDR3 of SEQ ID NOs: 335, 336, and 337, respectively.

In one embodiment, the present disclosure provides a nucleic acid molecule or nucleic acid molecules that comprise a nucleotide sequence encoding the HCVR sequence of the anti-CD20 antigen-binding domain comprising SEQ ID NO: 329, a nucleotide sequence encoding the HCVR sequence of the anti-CD3 antigen-binding domain comprising SEQ ID NO: 331, and a nucleotide sequence encoding the LCVR sequence comprising SEQ ID NO: 330.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

### C. Characterization of CD20×CD3 Bispecific Antigen-Binding Molecules

The present disclosure includes bispecific antigen-binding molecules (e.g., bispecific antibodies) and functional fragments thereof that bind to CD20 and CD3 (e.g., human CD20 and CD3) with high affinity.

In some embodiments, the present disclosure includes bispecific antigen-binding molecules (e.g., bispecific antibodies as disclosed herein) that specifically interact (e.g., bind with) cells that express CD20 and/or CD3. The extent to which an antigen-binding molecule binds cells that express CD20 and/or CD3 can be assessed by an in vitro binding assay. For example, in some embodiments, the present disclosure provides anti-CD20×CD3 bispecific antibodies that specifically bind cells that express CD20 and/or CD3 on the cell surface (e.g., human B cells and/or T cells). In certain embodiments, the anti-CD20×CD3 bispecific antibodies bind Jurkat cells and Raji cells with an EC₅₀ value of less than about 60 nM, as measured by an in vitro binding assay. In certain embodiments, the anti-CD20×CD3 bispecific antibodies bind CD3 or CD20 on the surface of a Jurkat or Raji cell, respectively, with an EC₅₀ value of less than about 1000 mM, less than about 500 nM, less than about 200 nM, less than about 100 nM, less than about 75 nM, less than about 70 nM, less than about 65 nM, less than about 60 nM, less than about 50 nM, less than about 40 nM, less than about 30 nM, less than about 25 nM, less than about 10 nM, less than about 5 nM, less than about 2 nM, less than about 1 nM, less than about 500 pM, less than about 100 pM, less than about 10 pM, or less than about 1 pM as measured by an in vitro binding assay.

### V. Sequence Variants

The antigen-binding molecules of the present disclosure may comprise one or more amino acid substitutions, insertions, and/or deletions in the framework and/or CDR regions of the heavy and/or light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired property such as, improved binding specificity, increased binding affinity, improved, or enhanced antagonistic or agonistic biological properties, reduced immunogenicity, etc. Bispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. In some embodiments, the disclosure includes use of an antibody having HCVR, LCVR, and/or CDR amino acid sequences with 1, 2, 3, or 4 conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate; and (7) sulfur-containing side chains are cysteine and methionine. Preferred conservative amino acids substitution groups are valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antigen-binding molecules comprising an antigen binding domain with a HCVR, LCVR, and/or CDR amino acid sequence that is substantially identical to any of the HCVR, LCVR, and/or CDR amino acid sequences disclosed herein. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 46. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 46, wherein the differences in the amino acid residue(s) relative to the sequence disclosed in Table 46 are conservative substitutions or moderately conservative substitutions. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1, wherein the differences in the amino acid residue(s) relative to the sequence disclosed in Table 1 are conservative substitutions or moderately conservative substitutions.

### VI. Antigen-Binding Proteins Comprising Fc Modifications

In some embodiments, an antigen-binding molecule as disclosed herein (e.g., a CD40 antigen-binding molecule such as a CD40×CD40 bispecific antigen-binding molecule, e.g., as disclosed in any one of Tables 9-12, a BCMA×CD3 bispecific antigen-binding molecule such as an anti-BCMA×CD3 bispecific antibody, or a CD20×CD3 bispecific antigen-binding molecule such as an anti-CD20×CD3 bispecific antibody) comprises an Fc domain comprising one or more modifications or mutations that enhance or diminish antibody binding to the FcRn receptor. For example, the present disclosure includes antigen-binding molecules comprising one or more mutations in the CH2 and/or CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). See, e.g., Ko et al., BioDrugs 2021, 35:147-157.

In certain embodiments, a CD40×CD40 bispecific antigen-binding molecule, a BCMA×CD3 bispecific antigen-binding molecule, or a CD20×CD3 bispecific antigen-binding molecule comprises an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). In some embodiments, the human IgG heavy chain constant region comprises one or more modifications that increase binding to a neonatal Fc receptor (FcRn). For example, in some embodiments the human IgG heavy chain constant region comprises M252Y, S254T, and T256E mutations.

In some embodiments, the CD40×CD40 bispecific antigen-binding molecule, the BCMA×CD3 bispecific antigen-binding molecules, or the CD20×CD3 bispecific antigen-binding molecules of the present disclosure comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, a modified Fc domain having reduced binding to an Fc receptor, such as an Fc-gamma receptor (e.g., Fcγ receptor, e.g., FcγRI, FcγRIIA, FcγRIIB, or FcγRIIIA), is a variant IgG1 Fc or a variant IgG4 Fc comprising one or more substitutions or modifications in the hinge region and/or a CH region (e.g., CH2). For example, a modified Fc domain may comprise a variant IgG1 Fc wherein at least one amino acid of an IgG1 Fc hinge region and/or CH region is replaced with the corresponding amino acid from an IgG2 Fc hinge region and/or CH region. In certain embodiments, the modified Fc domain is a variant IgG1 Fc or a variant IgG4 Fc comprising one or more substitutions or modifications in the hinge region. For example, a modified Fc domain may comprise a variant IgG1 Fc wherein at least one amino acid of the IgG1 Fc hinge region is replaced with the corresponding amino acid from the IgG2 Fc hinge region. In one example, the variant IgG1 Fc can comprise a human IgG2 lower hinge amino acid sequence or can comprise both a human IgG2 lower hinge amino acid sequence and a human IgG4 CH2 amino acid sequence. For example, in some embodiments, the heavy chain constant region can comprise a variant IgG1 Fc in which positions 233-236 by EU numbering are occupied by PVA. See, e.g., U.S. Pat. No. 10,988,537, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by PVA. Alternatively, a modified Fc domain may comprise a variant IgG4 Fc wherein at least one amino acid of an IgG4 Fc hinge region and/or CH region is replaced with the corresponding amino acid from an IgG2 Fc hinge region and/or CH region. Alternatively, a modified Fc domain may comprise a variant IgG4 Fc wherein at least one amino acid of the IgG4 Fc hinge region is replaced with the corresponding amino acid from the IgG2 Fc hinge region. In one example, the variant IgG4 Fc can comprise a human IgG2 lower hinge amino acid sequence. For example, in some embodiments, the heavy chain constant region can comprise a variant IgG4 Fc in which positions 233-236 by EU numbering are occupied by PVA. See, e.g., U.S. Pat. No. 10,988,537, the disclosure of which is hereby incorporated by reference in its entirety. In some embodiments, a modified Fc domain comprises a modified hinge region in which each of positions 233-236 by EU numbering is occupied by G or is unoccupied. In some embodiments, a modified Fc domain comprises modifications in which each of positions 233-236 by EU numbering is occupied by G or is unoccupied. For example, in some embodiments, a modified Fc domain can comprise a modified hinge region in which positions 233-236 by EU numbering are occupied by GGG. See, e.g., U.S. Pat. No. 11,518,807, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by GGG. Non-limiting, exemplary modified Fc regions that can be used in the context of the present disclosure are set forth in U.S. Pat. No. 11,518,807, the disclosure of which is hereby incorporated by reference in its entirety, as well as any functionally equivalent variants of the modified Fc regions set forth therein. Other modified Fe domains and Fc modifications that can be used in the context of the present disclosure include any of the modifications as set forth in U.S. Pat. Nos. 8,697,396, 10,988,537, US 2014/0171623, US 2014/0134162, US 2014/0243504, and WO 2014/043361, the disclosures of each of which are incorporated by reference herein.

In certain embodiments, a bispecific antigen-binding molecule as disclosed herein comprises immunoglobulin heavy chains that are heterodimeric (i.e., differing by at least one amino acid) and have differential affinity toward an affinity reagent, such as Protein A. In some embodiments, one of the heavy chains comprises one or more modifications in the Fc domain that reduces or eliminates binding of the Fc domain to Protein A. In some embodiments, one of the heavy chains comprises H435R/Y436F (by EU numbering system) substitutions in the CH3 region. Non-limiting, exemplary modified Fc regions that can be used in the context of the present disclosure are set forth in U.S. Pat. No. 8,586,713, the disclosure of which is hereby incorporated by reference in its entirety.

All possible combinations of the foregoing Fc domain mutations, and other mutations within the antibody variable domains disclosed herein, are contemplated within the scope of the present disclosure.

### VII. Epitope Mapping and Related Technologies

In some embodiments, the epitope on CD40 to which the antigen-binding molecules of the present disclosure bind (e.g., a first epitope of human CD40 to which a first antigen-binding domain (D1) binds, or a second epitope of human CD40 to which a second antigen-binding domain (D2) binds) may consist of a single contiguous sequence of 3 or more (e.g., 3, 4, 5, 6, 7, 8, 9, 10, 11, 12, 13, 14, 15, 16, 17, 18, 19, 20 or more) amino acids of a CD40 protein. Alternatively, the epitope may consist of a plurality of non-contiguous amino acids (or amino acid sequences) of a CD40 protein. In some embodiments, the epitope on BCMA and/or CD20 and/or CD3 to which the antigen-binding molecules of the present disclosure bind (e.g., an epitope of BCMA or CD20 to which a first antigen-binding domain (D1) binds, or an epitope of CD3 to which a second antigen-binding domain (D2) binds) may consist of a single contiguous sequence of 3 or more (e.g., 3, 4, 5, 6, 7, 8, 9, 10, 11, 12, 13, 14, 15, 16, 17, 18, 19, 20 or more) amino acids of a BCMA or CD20 or CD3 protein. Alternatively, the epitope may consist of a plurality of non-contiguous amino acids (or amino acid sequences) of a BCMA or CD20 or CD3 protein. The antibodies of the invention may interact with amino acids contained within a single CD3 chain (e.g., CD3-epsilon, CD3-delta or CD3-gamma), or may interact with amino acids on two or more different CD3 chains. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antibody interacts.

In some embodiments, the present disclosure includes anti-CD40 antibodies and anti-CD40×CD40 bispecific antibodies that bind to the same epitope or epitopes as any of the specific exemplary antibodies described herein (e.g., antibodies comprising any of the amino acid sequences set forth in Table 46 below). In some embodiments, the present disclosure includes anti-CD40 antibodies and anti-CD40×CD40 bispecific antibodies that compete for binding with the exemplary antibodies described herein (e.g., antibodies comprising any of the amino acid sequences set forth in Table 46 below).

In some embodiments, the present disclosure provides anti-CD40×CD40 bispecific antibodies comprising a first antigen-binding domain (D1) that binds a first epitope of human CD40 and a second antigen-binding domain (D1) that binds a second epitope of human CD40, wherein the first epitope and the second epitope are different epitopes of CD40. In some embodiments, the first epitope and the second epitope are non-overlapping epitopes.

In some embodiments, the present disclosure provides anti-CD40×CD40 bispecific antibodies comprising a first antigen-binding domain (D1) that binds a first epitope of human CD40 and a second antigen-binding domain (D1) that binds a second epitope of human CD40, wherein D1 and D2 do not compete with one another for binding to human CD40.

The present disclosure also includes antigen-binding molecules (e.g., antibodies or antigen-binding domains thereof) that bind to the same epitope as, or competes for binding with, a bispecific BCMA×CD3 antigen-binding molecule or a bispecific CD20×CD3 antigen-binding molecule described herein.

One skilled in the art can determine whether or not a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on CD40 or the same epitope on BCMA and/or CD20 and/or CD3 as a reference bispecific antigen-binding molecule of the present disclosure, the reference bispecific molecule is first allowed to bind to a CD40 protein or bind to a BCMA and/or CD20 and/or CD3 protein. Next, the ability of a test antibody to bind to the CD40 molecule or bind to the BCMA and/or CD20 and/or CD3 molecule is assessed. If the test antibody is able to bind to CD40 or bind to BCMA and/or CD20 and/or CD3 following saturation binding with the reference bispecific antigen-binding molecule, it can be concluded that the test antibody binds to a different epitope of CD40 or a different epitope of BCMA and/or CD20 and/or CD3 than the reference bispecific antigen-binding molecule. On the other hand, if the test antibody is not able to bind to the CD40 molecule or not able to bind to the BCMA and/or CD20 and/or CD3 molecule following saturation binding with the reference bispecific antigen-binding molecule, then the test antibody may bind to the same epitope of CD40 or the same epitope of BCMA and/or CD20 and/or CD3 as the epitope bound by the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, radioimmunoassay (RIA), Biacore, flow cytometry or any other quantitative or qualitative antibody-binding assay available in the art. In accordance with certain embodiments of the present disclosure, two antigen-binding proteins bind to the same (or overlapping) epitope if, e.g., a 1-, 2-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CD40 protein or bind to a BCMA and/or CD20 and/or CD3 protein under saturating conditions followed by assessment of binding of the test antibody to the CD40 molecule or binding of the test antibody to the BCMA and/or CD20 and/or CD3 molecule. In a second orientation, the test antibody is allowed to bind to a CD40 molecule or bind to a BCMA and/or CD20 and/or CD3 molecule under saturating conditions followed by assessment of binding of the reference antigen-binding molecule to the CD40 molecule or to the BCMA and/or CD20 and/or CD3 molecule. If, in both orientations, only the first (saturating) antigen-binding molecule is capable of binding to the CD40 molecule or binding to the BCMA and/or CD20 and/or CD3 molecule, then it is concluded that the test antibody and the reference antigen-binding molecule compete for binding to CD40 or binding to BCMA and/or CD20 and/or CD3. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### VIII. Preparation of Antigen-Binding Domains and Construction of Multispecific Antigen-Binding Molecules

Antigen-binding domains specific for particular antigens can be prepared by any antibody generating technology known in the art. Once obtained, two different antigen-binding domains can be appropriately arranged relative to one another to produce a bispecific antigen-binding molecule of the present disclosure using routine methods. A discussion of exemplary bispecific antibody formats that can be used to construct the bispecific antigen-binding molecules of the present disclosure is provided elsewhere herein. In certain embodiments, one or more of the individual components (e.g., heavy, and light chains) of the multispecific antigen-binding molecules are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., CD40 or BCMA or CD20 or CD3) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules.

In some embodiments, genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. See, e.g., US 2011/0195454, the entire contents of which are incorporated herein by reference, for a detailed discussion of such engineered mice and the use thereof to produce bispecific antigen-binding molecules. As used herein, “fully human” refers to an antigen-binding molecule, e.g., an antibody, or antigen-binding fragment or immunoglobulin domain thereof, comprising an amino acid sequence encoded by a DNA derived from a human sequence over the entire length of each polypeptide of the antigen-binding molecule, antibody, antigen-binding fragment, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### IX. Bioequivalents

The present disclosure encompasses antigen-binding molecules having amino acid sequences that vary from those of the described antibodies but that retain the ability to bind CD40 or retain the ability to bind BCMA and/or CD20 and/or CD3. Such variant molecules comprise one or more additions, deletions, or substitutions of amino acids when compared to the parent sequence, but exhibit biological activity that is essentially equivalent to that of the described antigen-binding molecules. Likewise, the nucleic acid sequences encoding the antigen-binding molecules of the present disclosure encompass sequences that comprise one or more additions, deletions, or substitutions of nucleotides when compared to the disclosed sequence, but that encode an antigen-binding molecule that is essentially bioequivalent to the antigen-binding molecules disclosed herein.

The present disclosure includes antigen-binding molecules that are bioequivalent to any of the exemplary antigen-binding molecules set forth herein. Two antigen-binding proteins, e.g., bispecific antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the first antigen-binding protein (e.g., reference product) and the second antigen-binding protein (e.g., biological product) without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other embodiments, bioequivalent antibodies may include the exemplary bispecific antigen-binding molecules set forth herein comprising amino acid changes which modify the glycosylation characteristics of the antibodies, e.g., mutations which eliminate or remove glycosylation.

### X. Immunoglobulin Depleting Agents

In various aspects, the present disclosure provides immunoglobulin depleting agents combined with or administered to subjects with preexisting immunity against an immunogen (i.e., an immunogen to be administered to the subject, e.g., an immunogenic delivery vehicle such as, e.g., AAV) in combination with plasma cell depleting agents (e.g., an anti-BCMA×CD3 bispecific antibody, or a functional fragment thereof) or B cell depleting agents (e.g., an anti-CD20×CD3 bispecific antibody, or a functional fragment thereof) described herein. For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. In some embodiments, the immunoglobulin depleting agent is administered to subjects with preexisting immunity against an immunogen (i.e., an immunogen to be administered to the subject, e.g., an immunogenic delivery vehicle such as, e.g., AAV) not only in combination with a plasma cell depleting agent but also in combination with a B cell depleting agent, plasmapheresis, therapeutic plasma exchange, immunoadsorption, and/or an immunogen (e.g., nucleic acid construct, nuclease agent or CRISPR/Cas system, e.g., in an immunogenic delivery vehicle) (e.g., an immunogenic delivery vehicle such as, e.g., AAV) disclosed herein. Suitable combinations comprising a plasma cell depleting agent are described in more detail elsewhere herein. In some embodiments, an immunoglobulin depleting agent may be useful for, e.g., accelerating IgG clearance.

In some embodiments, an immunoglobulin depleting agent is capable of accelerating IgG serum clearance.

In some embodiments, an immunoglobulin depleting agent may comprise a neonatal Fc receptor (FcRn) blocker such as, but not limited to, efgartigimod alfa. The mechanistic concept of FcRn-targeting therapeutics is to accelerate IgG catabolism by blocking the FcRn-mediated intracellular IgG recycling pathway, thereby reducing overall plasma IgG levels. FcRn can participate in the maintenance of IgG levels by salvaging IgG from lysosomal degradation, thereby prolonging the half-life of IgG. In some embodiments, FcRn blockers can compete with IgG for binding to FcRn. Due to their higher affinity for FcRn, FcRn blockers can prevent IgG from binding to FcRn and, instead, IgG is transported to the lysosome and degraded, thereby leading to decreased circulating levels of IgG.

In some embodiments, an FcRn blocker can include Efgartigimod (ARGX-113), Rozanolixizumab (UCB7665), Batoclimab (RVT-1401), IMVT-1402, Nipocalimab (M281), Orilanolimab (SYNT001), or any combination thereof. See, e.g., Zuercher et al. (2019) Autoimmun. Rev. 18(10):102366.

In some embodiments, the immunoglobulin depleting agent is an immunoglobulin degrading enzyme (e.g., IgG degrading enzyme). In some embodiments, an immunoglobulin depleting agent may comprise an IgG degrading enzyme such as IdeS (imlifidase), IdeZ, or IdeXork. IdeS (imlifidase) is an endopeptidase derived from Streptococcus pyogenes which has specificity for human IgG, and when infused intravenously results in rapid cleavage of IgG. IdeZ (immunoglobulin-degrading enzyme from Streptococcus equi subspecies zooepidemicus) is an engineered recombinant protease overexpressed in Escherichia coli. IdeZ specifically cleaves IgG molecules below the hinge region to yield F(ab′)2 and Fc fragments. IdeXork (Xork) is yet another example of an IgG protease. Additional non-limiting examples of IgG degrading enzymes include Imlifidase/IdeS/Fabricator, IdeZ, IceMG (IgM and IgG degrading enzyme, whereas IceM is an IgM degrading enzyme), CYR-212, CYR-241, 5-1117, HNSA-5487, Xork, and IdeE/KJ103. In some embodiments, an immunoglobulin depleting agent may facilitate IgG degradation via lysosomal destruction. A non-limiting example of an immunoglobulin depleting agent which may facilitate IgG degradation via lysosomal destruction is BHV-1300.

### XI. Plasmapheresis, Therapeutic Plasma Exchange, and Immunoadsorption

In various aspects, the methods disclosed herein can include plasmapheresis, therapeutic plasma exchange, or immunoadsorption in subjects with preexisting immunity against an immunogen (i.e., an immunogen to be administered to the subject, e.g., an immunogenic delivery vehicle such as, e.g., AAV). For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. These can be combined, for example, with treatment with plasma cell depleting agents (e.g., an anti-BCMA×CD3 bispecific antibody, or a functional fragment thereof) or with not only treatment with plasma cell depleting agents but also treatment with B cell depleting agents (e.g., an anti-CD20×CD3 bispecific antibody, or a functional fragment thereof), and/or immunoglobulin depleting agents described herein in subjects with preexisting immunity against an immunogen (i.e., an immunogen to be administered to the subject, e.g., an immunogenic delivery vehicle such as, e.g., AAV). In some embodiments, the plasmapheresis, therapeutic plasma exchange, or immunoadsorption may be performed in combination with treatment with a plasma cell depleting agent or in combination with treatment with a plasma cell depleting agent and a B cell depleting agent, and/or an immunogen (e.g., nucleic acid construct, nuclease agent or CRISPR/Cas system, e.g., in an immunogenic delivery vehicle) disclosed herein in subjects with preexisting immunity against an immunogen (i.e., an immunogen to be administered to the subject, e.g., an immunogenic delivery vehicle such as, e.g., AAV). Suitable combinations comprising a plasma cell depleting agent are described in more detail elsewhere herein. Plasmapheresis, therapeutic plasma exchange, and immunoadsorption may be useful strategies for removal of AAV antibodies from patients' blood plasma.

Plasmapheresis is a process used to selectively remove blood components used to treat a variety of conditions including those caused by the acute overproduction of antibodies (e.g., autoimmunity, transplant rejection), in which removal of pathogenic immunoglobulins results in clinical benefit. Immunoadsorption is a selective therapeutic apheresis technique by which immunoglobulins are selectively removed from patients' plasma. The immunoadsorption can be, for example, total immunoglobulin immunoadsorption. See, e.g., Boedecker-Lips et al. (2023) J. Clin. Apher. 38(5):590-601. Alternatively, the immunoadsorption can be AAV capsid specific immunoadsorption. See, e.g., Bertin et al. (2020) Sci. Rep. 10:864.

### XII. Combinations Comprising a Plasma Cell Depleting Agent

A plasma cell depleting agent (e.g., a BCMA×CD3 antigen-binding molecule) can be administered either alone, or in combination with, a B cell depleting agent (e.g., a CD20×CD3 antigen-binding molecule), an immunoglobulin depleting agent (e.g., an FcRn blocker, such as Efgartigimod), and/or an immunogen. For example, the immunogen can be a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. In some embodiments, the administration of the plasma cell depleting (and optionally the B cell depleting agent, the immunoglobulin depleting agent, and/or the immunogen) can be further combined with plasmapheresis, therapeutic plasma exchange, and/or immunoadsorption to a subject in need thereof with preexisting immunity against an immunogen (i.e., an immunogen to be administered to the subject, e.g., an immunogenic delivery vehicle such as, e.g., AAV). Any such plasma cell depleting agents or combinations can also be administered in combination with a CD40 inhibitor in scenarios such as those described in detail elsewhere herein. Such scenarios include, for example: (1) a plasma cell depleting agent or a combination comprising a plasma cell depleting agent as disclosed herein [e.g., in combination with a B cell depleting agent (e.g., a CD20×CD3 antigen-binding molecule) and/or an immunoglobulin depleting agent and/or plasmapheresis, therapeutic plasma exchange, or immunoadsorption] is used to eliminate preexisting immunity to an immunogen (e.g., AAV), while a CD40 inhibitor is used to prevent any new antibody response to the immunogen on subsequent immunogen exposure; (2) a plasma cell depleting agent or a combination comprising a plasma cell depleting agent as disclosed herein is used to eliminate potential residual antibody responses generated following immunogen (e.g., AAV) exposure in the presence of CD40 blockade (e.g., if CD40 blockade is not completely effective); or (3) CD40 blockade is used concurrently with a plasma cell depleting agent or a combination comprising a plasma cell depleting agent as disclosed herein to block ongoing antibody responses to an immunogen (e.g., AAV) from recent exposure. As used herein, the term “in combination with,” e.g., a plasma cell depleting agent (or any other compound) means that additional component(s) may be administered prior to, concurrent with, or after the administration of the plasma cell depleting agent (or any other compound, such as other immunomodulator or immunogen). The different components of the combination can be formulated into a single composition, e.g., for simultaneous delivery, or formulated separately into two or more compositions (e.g., a kit including each component, for example, wherein the further agent is in a separate formulation).

For example, a plasma cell depleting agent (e.g., a BCMA×CD3 antigen-binding molecule) can be administered either alone, or in combination with, a B cell depleting agent (e.g., a CD20×CD3 antigen-binding molecule) and/or an immunoglobulin depleting agent (e.g., an FcRn blocker, such as Efgartigimod) to a subject in need thereof with preexisting immunity against an immunogen (i.e., an immunogen to be administered to the subject, e.g., an immunogenic delivery vehicle such as, e.g., AAV). In some embodiments, the B cell depleting agent is administered before, at the same time as, or after the plasma cell depleting agent. In some embodiments, the immunoglobulin depleting agent is administered after the plasma cell depleting agent. In some embodiments, the B cell depleting agent is administered prior to and after the nucleic acid construct. In some embodiments, the immunoglobulin depleting agent is administered prior to and after the nucleic acid construct. In some embodiments, the immunoglobulin depleting agent is administered after an initial dose of the plasma cell depleting agent, or wherein the immunoglobulin depleting agent is administered after an initial dose of the plasma cell depleting agent and after an initial dose of the B cell depleting agent.

In one example, a plasma cell depleting agent (e.g., a BCMA×CD3 antigen-binding molecule) is administered in combination with a B cell depleting agent (e.g., a CD20×CD3 antigen-binding molecule) to a subject in need thereof with preexisting immunity against an immunogen (i.e., an immunogen to be administered to the subject, e.g., an immunogenic delivery vehicle such as, e.g., AAV).

In another example, a plasma cell depleting agent (e.g., a BCMA×CD3 antigen-binding molecule) is administered in combination with an immunoglobulin depleting agent (e.g., an FcRn blocker, such as Efgartigimod) to a subject in need thereof with preexisting immunity against an immunogen (i.e., an immunogen to be administered to the subject, e.g., an immunogenic delivery vehicle such as, e.g., AAV). In some embodiments, the immunoglobulin depleting agent comprises an FcRn blocker. In some embodiments, the immunoglobulin depleting agent comprises an IgG degrading enzyme.

In some embodiments, the combination of the plasma cell depleting agent and the immunoglobulin depleting agent, when administered in further combination with an immunogen (e.g., an immunogenic delivery vehicle such as, e.g., AAV) to a subject in need thereof, decreases a level of an anti-immunogen antibody titer (e.g., an anti-AAV antibody titer) in the subject (e.g., such as can be measured in a serum sample isolated from the subject). In some embodiments, the level of the anti-immunogen antibody titer is decreased by about 1-fold to about 20-fold, about 2-fold to about 15-fold, about 4-fold to about 10-fold, about 3-fold to about 18-fold, about 5-fold to about 12-fold, or about 6-fold to about 8-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold, or more. In some embodiments, the anti-immunogen antibody titer is decreased by about 20-fold.

In another example, a plasma cell depleting agent (e.g., a BCMA×CD3 antigen-binding molecule) is administered in combination with a B cell depleting agent (e.g., a CD20×CD3 antigen-binding molecule) and an immunoglobulin depleting agent (e.g., an FcRn blocker, such as Efgartigimod) to a subject in need thereof with preexisting immunity against an immunogen (i.e., an immunogen to be administered to the subject, e.g., an immunogenic delivery vehicle such as, e.g., AAV). In some embodiments, the immunoglobulin depleting agent comprises an FcRn blocker. In some embodiments, the immunoglobulin depleting agent comprises an IgG degrading enzyme.

In some embodiments, the combination of the plasma cell-depleting agent, the B cell depleting agent, and the immunoglobulin-depleting agent, when administered in further combination with an immunogen (e.g., an immunogenic delivery vehicle such as, e.g., AAV) to a subject in need thereof, decreases the level of an anti-immunogen antibody titer (e.g., an anti-AAV antibody titer) in the subject (e.g., such as can be measured in a serum sample isolated from the subject). In some embodiments, the level of the anti-immunogen antibody titer may be decreased by about 1-fold to about 20-fold, about 2-fold to about 15-fold, about 4-fold to about 10-fold, about 3-fold to about 18-fold, about 5-fold to about 12-fold, about 6-fold to about 8-fold, about 10-fold to about 30-fold, about 20-fold to about 50-fold, about 30-fold to about 70-fold, about 40-fold to about 90-fold, or about 50-fold to about 100-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold, or more. In some embodiments, the anti-immunogen antibody titer is decreased by about 100-fold.

In another example, a plasma cell depleting agent (e.g., a BCMA×CD3 antigen-binding molecule) is administered in combination with plasmapheresis, therapeutic plasma exchange, or immunoadsorption to a subject in need thereof with preexisting immunity against an immunogen (i.e., an immunogen to be administered to the subject, e.g., an immunogenic delivery vehicle such as, e.g., AAV).

In another example, a plasma cell depleting agent (e.g., a BCMA×CD3 antigen-binding molecule) is administered in combination with plasmapheresis, therapeutic plasma exchange, or immunoadsorption and a B cell depleting agent (e.g., a CD20×CD3 antigen-binding molecule) to a subject in need thereof with preexisting immunity against an immunogen (i.e., an immunogen to be administered to the subject, e.g., an immunogenic delivery vehicle such as, e.g., AAV).

In another example, a plasma cell depleting agent (e.g., a BCMA×CD3 antigen-binding molecule) is administered in combination with plasmapheresis, therapeutic plasma exchange, or immunoadsorption and an immunoglobulin depleting agent (e.g., an FcRn blocker, such as Efgartigimod) to a subject in need thereof with preexisting immunity against an immunogen (i.e., an immunogen to be administered to the subject, e.g., an immunogenic delivery vehicle such as, e.g., AAV). In some embodiments, the immunoglobulin depleting agent comprises an FcRn blocker. In some embodiments, the immunoglobulin depleting agent comprises an IgG degrading enzyme.

In another example, a plasma cell depleting agent (e.g., a BCMA×CD3 antigen-binding molecule) is administered in combination with plasmapheresis, therapeutic plasma exchange, or immunoadsorption, a B cell depleting agent (e.g., a CD20×CD3 antigen-binding molecule), and an immunoglobulin depleting agent (e.g., an FcRn blocker, such as Efgartigimod) to a subject in need thereof with preexisting immunity against an immunogen (i.e., an immunogen to be administered to the subject, e.g., an immunogenic delivery vehicle such as, e.g., AAV). In some embodiments, the immunoglobulin depleting agent comprises an FcRn blocker. In some embodiments, the immunoglobulin depleting agent comprises an IgG degrading enzyme.

In some embodiments, the B cell depleting agent to be used in combination with the plasma cell depleting agent in subjects with preexisting immunity against an immunogen (i.e., an immunogen to be administered to the subject, e.g., an immunogenic delivery vehicle such as, e.g., AAV) comprises two or more B cell depleting agents (e.g., an anti-CD19 antigen-binding molecule and an anti-CD20 antigen-binding molecule). In some embodiments, the immunoglobulin depleting agent to be used in combination with the plasma cell depleting agent in subjects with preexisting immunity against an immunogen (i.e., an immunogen to be administered to the subject, e.g., an immunogenic delivery vehicle such as, e.g., AAV) comprises two or more immunoglobulin depleting agents (e.g., an FcRn blocker and an IgG degrading enzyme).

In embodiments in which a plasma cell depleting agent (e.g., a BCMA×CD3 antigen-binding molecule) is administered in combination with a B cell depleting agent (e.g., a CD20×CD3 antigen-binding molecule) and/or an immunoglobulin depleting agent (e.g., an FcRn blocker, such as Efgartigimod) and/or plasmapheresis, therapeutic plasma exchange, or immunoadsorption to a subject in need thereof with preexisting immunity against an immunogen (i.e., an immunogen to be administered to the subject, e.g., an immunogenic delivery vehicle such as, e.g., AAV), one or more or all treatments can occur together or one or more or all treatments can occur sequentially. For example, in some embodiments in which the plasma cell depleting agent (e.g., a BCMA×CD3 antigen-binding molecule) is administered in combination with an IgG degrading enzyme to a subject in need thereof with preexisting immunity against an immunogen (i.e., an immunogen to be administered to the subject, e.g., an immunogenic delivery vehicle such as, e.g., AAV), the plasma cell depleting agent can be administered to the subject first, followed by the IgG degrading enzyme. In another example, in embodiments where an immunoglobulin depleting agent (e.g., FcRn blocker) is administered together with plasmapheresis, therapeutic plasma exchange, or immunoadsorption, the plasmapheresis, therapeutic plasma exchange, or immunoadsorption can be first followed by administration of the immunoglobulin depleting agent (e.g., FcRn blocker). Likewise, in embodiments in which a plasma cell depleting agent is administered in combination with a CD40 inhibitor, the treatments can occur together or one or more or all treatments can occur sequentially.

### XIII. Nucleic Acid Constructs Encoding a Polypeptide of Interest

The compositions and methods described herein include the use of a nucleic acid construct that comprises a coding sequence for a polypeptide of interest (e.g., an exogenous polypeptide coding sequence). The compositions and methods described herein can also include the use of a nucleic acid construct that comprises a polypeptide of interest coding sequence or a reverse complement of the polypeptide of interest coding sequence (e.g., an exogenous polypeptide coding sequence or a reverse complement of the exogenous polypeptide coding sequence). Such nucleic acid constructs can be for insertion into a target genomic locus or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a Spy Cas9 protein complexed with a Spy Cas9 guide RNA. In some cases, the polypeptide of interest is an exogenous polypeptide as defined herein.

In a specific example, the compositions and methods described herein include the use of a nucleic acid construct encoding a Factor IX protein. Such nucleic acid constructs can be for insertion into a target genomic locus following cleavage at a cleavage site by a nuclease agent or CRISPR/Cas system as disclosed elsewhere herein or can be for expression of the Factor IX protein without insertion into a target genomic locus or a cleavage site (e.g., in an episome). The term cleavage site includes a DNA sequence at which a nick or double-strand break is created by a nuclease agent (e.g., a Cas9 protein complexed with a guide RNA). In certain embodiments, the cleavage site includes a DNA sequence at which a double-strand break is created by a Cas9 protein complexed with a guide RNA, e.g., a Spy Cas9 protein complexed with a Spy Cas9 guide RNA.

In another specific example, the compositions and methods described herein include the use of a nucleic acid construct that comprises a multidomain therapeutic protein (e.g., GAA fusion protein) coding sequence (a multidomain therapeutic protein nucleic acid). See, e.g., PCT/US2023/061858 and U.S. Ser. No. 18/163,698, each of which is herein incorporated by reference in its entirety for all purposes. Such nucleic acid constructs can be for insertion into a target genomic locus following cleavage at a cleavage site by a nuclease agent or CRISPR/Cas system as disclosed elsewhere herein or can be for expression of the multidomain therapeutic protein without insertion into a target genomic locus or a cleavage site (e.g., in an episome).

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell's ALB locus). Such constructs may not comprise a promoter that drives the expression of the polypeptide of interest. For example, the expression of the polypeptide of interest can be driven by a promoter of the host cell (e.g., the endogenous ALB promoter when the transgene is integrated into a host cell's ALB locus). In such cases, the construct may lack control elements (e.g., promoter and/or enhancer) that drive its expression (e.g., a promoterless construct). Nonetheless, in other cases the construct may comprise a promoter and/or enhancer, for example, a constitutive promoter or an inducible or tissue-specific (e.g., liver- or platelet-specific) promoter that drives expression of the polypeptide of interest in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a polypeptide of interest downstream of and operably linked to a signal sequence encoding a signal peptide. In some examples, the nucleic acid construct works in homology-independent insertion of a nucleic acid that encodes a polypeptide of interest. Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans.

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a safe harbor gene, such as an ALB locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the polypeptide of interest coding sequence in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the polypeptide of interest coding sequence. In a specific example, the construct is delivered AAV-mediated delivery, and cleavage of the target sites flanking the polypeptide of interest coding sequence can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the polypeptide of interest coding sequence is inserted into the cut site or target DNA sequence in the correct orientation but it is reformed if the polypeptide of interest coding sequence is inserted into the cut site or target DNA sequence in the opposite orientation. This can help ensure that the polypeptide of interest coding sequence is inserted in the correct orientation for expression.

The constructs disclosed herein can comprise a polyadenylation sequence or polyadenylation tail sequence (e.g., downstream or 3′ of a polypeptide of interest coding sequence). Methods of designing a suitable polyadenylation tail sequence are well-known. The polyadenylation tail sequence can be encoded, for example, as a “poly-A” stretch downstream of the polypeptide of interest coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. In another example, the polyadenylation signal is a bovine growth hormone (BGH) polyadenylation signal or a CpG depleted BGH polyadenylation signal.

In one example, the polyadenylation signal can comprise a BGH polyadenylation signal. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. In a specific example, the BGH polyadenylation signal can be upstream (5′) of the SV40 polyadenylation signal (e.g., unidirectional SV40 late polyadenylation signal). In another example, the polyadenylation signal can comprise a combination of a BGH polyadenylation signal and a synthetic polyadenylation signal. In some embodiments, the nucleic acid construct is a unidirectional construct.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal.

The constructs disclosed herein may also comprise splice acceptor sites (e.g., operably linked to the polypeptide of interest coding sequence, such as upstream or 5′ of the polypeptide of interest coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest (e.g., a GAA splice acceptor). For example, such a splice acceptor can be derived from the human GAA gene. Alternatively, such a splice acceptor can be derived from the mouse GAA gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

### A. Polypeptides of Interest

Any polypeptide of interest may be encoded by the nucleic acid constructs disclosed herein. In one example, the polypeptide of interest is a therapeutic polypeptide (e.g., a polypeptide that is lacking or deficient in a subject). In one example, the polypeptide of interest is an enzyme.

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

The polypeptide of interest can be an exogenous polypeptide. An “exogenous” polypeptide coding sequence can refer to a coding sequence that has been introduced from an exogenous source to a site within a host cell genome (e.g., at a genomic locus such as a safe harbor locus, including ALB intron 1). That is, the exogenous polypeptide coding sequence is exogenous with respect to its insertion site, and the polypeptide of interest expressed from such an exogenous coding sequence is referred to as an exogenous polypeptide. The exogenous coding sequence can be naturally-occurring or engineered, and can be wild type or a variant. The exogenous coding sequence may include nucleotide sequences other than the sequence that encodes the exogenous polypeptide (e.g., an internal ribosomal entry site). The exogenous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The exogenous coding sequence can also be a coding sequence that is not naturally occurring in the host genome, or that expresses an exogenous polypeptide that does not naturally occur in the host genome. An exogenous coding sequence can include an exogenous nucleic acid sequence (e.g., a nucleic acid sequence is not endogenous to the recipient cell), or may be exogenous with respect to its insertion site and/or with respect to its recipient cell.

In one example, the polypeptide of interest is a factor IX protein. See, e.g., WO 2023/077012 and US 2023-0149563, each of which is herein incorporated by reference in its entirety for all purposes.

In one example, the polypeptide of interest is a multidomain therapeutic protein comprising a CD63-binding delivery domain linked to or fused to a lysosomal alpha-glucosidase (GAA). See, e.g., US 2023-0338477, WO 2023/150623, US 2025-0041455, and WO 2025/029657, each of which is herein incorporated by reference in its entirety for all purposes. In one example, the polypeptide of interest is a multidomain therapeutic protein comprising a CD63-binding delivery domain fused to a lysosomal alpha-glucosidase (GAA). In one example, the polypeptide of interest is a multidomain therapeutic protein comprising a TfR-binding delivery domain linked to or fused to a GAA. See, e.g., US 2023-0338477, WO 2023/150623, US 2025-0041455, and WO 2025/029657, each of which is herein incorporated by reference in its entirety for all purposes. In one example, the polypeptide of interest is a multidomain therapeutic protein comprising a TfR-binding delivery domain fused to a GAA.

In one example, the polypeptide of interest is a polypeptide associated with a genetic enzyme deficiency. In certain embodiments, the genetic enzyme deficiency results in infantile onset of disease. In certain embodiments, the genetic enzyme deficiency can be, or routinely is, diagnosed with newborn screening. In certain embodiments, the enzyme deficiency may manifest in various severity of disease such that the age of onset may include an infantile onset form of the disease and a later onset form of the disease (e.g., childhood, adolescent, or adult form of onset).

In one example, the polypeptide of interest is a polypeptide associated with a bleeding disorder, e.g., hemophilia, e.g., hemophilia A or hemophilia B. In certain embodiments, the polypeptide of interest is Factor VIII or Factor IX. In one example, the polypeptide of interest is an enzyme related to inborn errors of metabolism. In one example, the polypeptide of interest is an enzyme related to a lysosomal storage disease.

In another example, the polypeptide of interest is a multidomain therapeutic protein. A multidomain therapeutic protein as described herein includes a lysosomal alpha-glucosidase (GAA; e.g., to provide GAA enzyme replacement activity) linked to or fused to a delivery domain that provides binding to an internalization effector (a protein that is capable of being internalized into a cell or that otherwise participates in or contributes to retrograde membrane trafficking). Examples of multidomain therapeutic proteins can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes. For example, the multidomain therapeutic proteins described herein can comprise a CD63-binding delivery domain linked to or fused to a lysosomal alpha-glucosidase (GAA). CD63-binding domains and GAA are described in more detail below. The CD63-binding domain provides binding to the internalization factor CD63. The multidomain therapeutic protein is targeted to the muscle by targeting CD63, which is a rapidly internalizing protein highly expressed in the muscle. In some multidomain therapeutic proteins, the CD63-binding delivery domain is covalently linked to the GAA. The covalent linkage may be any type of covalent bond (i.e., any bond that involved sharing of electrons). In some cases, the covalent bond is a peptide bond between two amino acids, such that the GAA and the CD63-binding delivery domain in whole or in part form a continuous polypeptide chain, as in a fusion protein. In some cases, the GAA portion and the CD63-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the CD63-binding delivery domain and the GAA to facilitate removal of the CD63-binding delivery domain in the lysosome.

In a particular multidomain therapeutic protein, the GAA is covalently linked to the C-terminus of the heavy chain of an anti-CD63 antibody or to the C-terminus of the light chain. In another particular multidomain therapeutic protein, the GAA is covalently linked to the N-terminus of the heavy chain of an anti-CD63 antibody or to the N-terminus of the light chain. In another particular embodiment, the GAA is linked to the C-terminus of an anti-CD63 scFv domain.

As another example, the multidomain therapeutic proteins described herein can comprise a TfR-binding delivery domain linked to or fused to a lysosomal alpha-glucosidase (GAA). TfR-binding domains and GAA are described in more detail below. The TfR-binding domain provides binding to the internalization factor TfR. The multidomain therapeutic protein produced by the liver is targeted the muscle and CNS by targeting TfR, which is expressed in muscle and on brain endothelial cells. Transcytosis of TfR in these cells enables blood-brain-barrier crossing. In some multidomain therapeutic proteins, the TfR-binding delivery domain is covalently linked to the GAA. The covalent linkage may be any type of covalent bond (i.e., any bond that involved sharing of electrons). In some cases, the covalent bond is a peptide bond between two amino acids, such that the GAA and the TfR-binding delivery domain in whole or in part form a continuous polypeptide chain, as in a fusion protein. In some cases, the GAA portion and the TfR-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the TfR-binding delivery domain and the GAA to facilitate removal of the TfR-binding delivery domain in the lysosome. See, e.g., US 2023-0338477, WO 2023/150623, US 2025-0041455, and WO 2025/029657, each of which is herein incorporated by reference in its entirety for all purposes.

In a particular multidomain therapeutic protein, the GAA is covalently linked to the C-terminus of the heavy chain of an anti-TfR antibody or to the C-terminus of the light chain. In another particular multidomain therapeutic protein, the GAA is covalently linked to the N-terminus of the heavy chain of an anti-TfR antibody or to the N-terminus of the light chain. In another particular embodiment, the GAA is linked to the C-terminus of an anti-TfR scFv domain.

In another example, the polypeptide of interest is an antigen-binding protein. See, e.g., WO 2020/206162 and US 2020-0318136, each of which is herein incorporated by reference in its entirety for all purposes. An “antigen-binding protein” as disclosed herein includes any protein that binds to an antigen. Examples of antigen-binding proteins include an antibody, an antigen-binding fragment of an antibody, a multi-specific antibody (e.g., a bi-specific antibody), an scFv, a bis-scFv, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus. For example, broadly neutralizing antibodies can focus on conserved functional targets, attacking a vulnerable site on conserved bacterial or viral proteins (e.g., a vulnerable site on the influenza viral protein hemagglutinin). Antibodies developed by the immune system upon infection or vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. Broadly neutralizing antibodies—termed “broadly” because they attack many strains of the bacteria or virus, and “neutralizing” because they attack key functional sites in the bacteria or virus and block infection—can overcome these problems. Unfortunately, however, these antibodies usually come too late and do not provide effective protection from the disease.

The antigen-binding proteins disclosed herein can target any antigen. The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

As one example, the targeted antigen can be a disease-associated antigen. The term “disease-associated antigen” refers to an antigen whose presence is correlated with the occurrence or progression of a particular disease. For example, the antigen can be in a disease-associated protein (i.e., a protein whose expression is correlated with the occurrence or progression of the disease). Optionally, a disease-associated protein can be a protein that is expressed in a particular type of disease but is not normally expressed in healthy adult tissue (i.e., a protein with disease-specific expression or disease-restricted expression). However, a disease-associated protein does not have to have disease-specific or disease-restricted expression.

As one example, a disease-associated antigen can be a cancer-associated antigen. The term “cancer-associated antigen” refers to an antigen whose presence is correlated with the occurrence or progression of one or more types of cancer. For example, the antigen can be in a cancer-associated protein (i.e., a protein whose expression is correlated with the occurrence or progression of one or more types of cancer). For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Optionally, a cancer-associated protein can be a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer.

As another example, a disease-associated antigen can be an infectious-disease-associated antigen. The term “infectious-disease-associated antigen” refers to an antigen whose presence is correlated with the occurrence or progression of a particular infectious disease. For example, the antigen can be in an infectious-disease-associated protein (i.e., a protein whose expression is correlated with the occurrence or progression of the infectious disease). Optionally, an infectious-disease-associated protein can be a protein that is expressed in a particular type of infectious disease but is not normally expressed in healthy adult tissue (i.e., a protein with infectious-disease-specific expression or infectious-disease-restricted expression). However, an infectious-disease-associated protein does not have to have infectious-disease-specific or infectious-disease-restricted expression. For example, the antigen can be a viral antigen or a bacterial antigen. Such antigens include, for example, molecular structures on the surface of viruses or bacteria (e.g., viral proteins or bacterial proteins) that are recognized by the immune system and are capable of triggering an immune response.

Examples of viral antigens include antigens within proteins expressed by the Zika virus or influenza (flu) viruses. Zika is a virus spread to people primarily through the bite of an infected Aedes species mosquito (Ae. aegypti and Ae. Albopictus). Zika virus infection during pregnancy can cause microcephaly and other severe brain defects. For example, a Zika antigen can be, but is not limited to, an antigen within a Zika virus envelope (Env) protein. Influenza virus is a virus that causes an infectious disease called influenza (commonly known as “the flu”). Three types of influenza viruses affect people, called Type A, Type B, and Type C. An influenza antigen can be, but is not limited to, an antigen within the hemagglutinin protein. Viral antigens and bacterial antigens also include antigens on other viruses and other bacteria. Examples of antibodies targeting influenza hemagglutinin are provided, e.g., in WO 2016/100807, herein incorporated by reference in its entirety for all purposes.

Examples of bacterial antigens include antigens within proteins expressed by Pseudomonas aeruginosa (e.g., an antigen within PcrV, which is a type III virulence system translocating protein). Pseudomonas aeruginosa is an opportunistic bacterial pathogen that causes fatal acute lung infections in critically ill individuals. Its pathogenesis is associated with bacterial virulence conferred by the type III secretion system (TTSS), through which P. aeruginosa causes necrosis of the lung epithelium and disseminates into the circulation, resulting in bacteremia, sepsis, and mortality. TTSS allows P. aeruginosa to directly translocate cytotoxins into eukaryotic cells, inducing cell death. The P. aeruginosa V-antigen PcrV, a homolog of the Yersinia V-antigen LcrV, is an indispensable contributor to TTS toxin translocation.

The antigen-binding protein can be a single-chain antigen-binding protein such as an scFv. Alternatively, the antigen-binding protein is not a single-chain antigen-binding protein. For example, the antigen-binding protein can include separate light and heavy chains. The heavy chain coding sequence can be upstream of the light chain coding sequence, or the light chain coding sequence can be upstream of the heavy chain coding sequence. In one specific example, the heavy chain coding sequence is upstream of the light chain coding sequence. For example, the heavy chain coding sequence can comprise V_{H}, D_{H}, and J_{H} segments, and the light chain coding sequence can comprise light chain V_{L} and light chain J_{L} gene segments. The antigen-binding protein coding sequence can be operably linked to an exogenous promoter in the nucleic acid construct, or the nucleic acid construct can be designed such that the antigen-binding protein coding sequence will be operably linked to an endogenous promoter at the genomic locus or safe harbor locus once it is genomically integrated. In one specific example, the nucleic acid construct can be designed such that the antigen-binding protein coding sequence will be operably linked to an endogenous promoter at the genomic locus or safe harbor locus once it is genomically integrated. Likewise, the antigen-binding protein coding sequence in the nucleic acid construct can include an exogenous signal sequence for secretion and/or the nucleic acid construct can be designed so that the antigen-binding protein coding sequence will be operably linked to an endogenous signal sequence at the genomic locus or safe harbor locus once it is genomically integrated. In one example, the nucleic acid construct can be designed so that the antigen-binding protein coding sequence will be operably linked to an endogenous signal sequence at the genomic locus or safe harbor locus once it is genomically integrated. In a specific example, the antigen-binding protein comprises separate light and heavy chains, and the nucleic acid construct is designed such that the coding sequence for one chain will be operably linked to an endogenous signal sequence at the genomic locus or safe harbor locus once it is genomically integrated and the coding sequence for the other chain is operably linked to a separate exogenous signal sequence. In a specific example, the antigen-binding protein comprises separate light and heavy chains, and the nucleic acid construct is designed such that the whichever chain coding sequence is upstream in the nucleic acid construct will be operably linked to an endogenous signal sequence at the genomic locus or safe harbor locus once it is genomically integrated, and an exogenous signal sequence is operably linked to the whichever chain coding sequence is downstream in the exogenous donor nucleic acid. Alternatively, the nucleic acid construct can be designed such that the coding sequences for both chains will be operably linked to an endogenous signal sequence at the genomic locus or safe harbor locus once it is genomically integrated, or the coding sequence for both chains can be operably linked to the same exogenous signal sequence or the coding sequence for each chain can be operably linked to separate exogenous signal sequences.

Signal sequences (i.e., N-terminal signal sequences) mediate targeting of nascent secretory and membrane proteins to the endoplasmic reticulum (ER) in a signal recognition particle (SRP)-dependent manner. Usually, signal sequences are cleaved off co-translationally so that signal peptides and mature proteins are generated. Examples of exogenous signal sequences or signal peptides that can be used include, for example, the signal sequence/peptide from mouse albumin, human albumin, mouse ROR1, human ROR1, human azurocidin, Cricetulus griseus Ig kappa chain V III region MOPC 63 like, and human Ig kappa chain V III region VG. Any other known signal sequence/peptide can also be used.

One or more of the nucleic acids in the antigen-binding-protein coding sequence (e.g., a heavy chain coding sequence and a light chain coding sequence) can be together in a multicistronic expression construct. For example, a nucleic acid encoding a heavy chain and a light chain can be together in a bicistronic expression construct. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin Biol Ther 5:627-638, herein incorporated by reference in its entirety for all purposes. Examples of 2A peptides that can be used include Thosea asigna virus 2A (T2A); porcine teschovirus-1 2A (P2A); equine rhinitis A virus (ERAV) 2A (E2A); and FMDV 2A (F2A). GSG residues can be added to the 5′ end of any of these peptides to improve cleavage efficiency.

In some nucleic acid constructs, a nucleic acid encoding a furin cleavage site is included between the light chain coding sequence and the heavy chain coding sequence. In some nucleic acid construct, a nucleic acid encoding a linker (e.g., GSG) is included between the light chain coding sequence and the heavy chain coding sequence (e.g., directly upstream of the 2A peptide coding sequence). For example, a furin cleavage site can be included upstream of a 2A peptide, with both the furin cleavage site and the 2A peptide being located between the light chain and the heavy chain (i.e., upstream chain-furin cleavage site-2A peptide-downstream chain). During translation, a first cleavage event will occur at the 2A peptide sequence. However, most of the 2A peptide will remain attached as a remnant to the C-terminus of the upstream chain (e.g., light chain if the light chain is upstream of the heavy chain, or heavy chain if the heavy chain is upstream of the light chain), with one amino acid added to the N-terminus of the downstream chain (or the N-terminus of a signal sequence, if a signal sequence is included upstream of the downstream chain). A second cleavage event, initiated at the furin cleavage site, yields the upstream chain without the 2A remnants in order to obtain a more native heavy chain or light chain by post-translational processing.

### (1) Factor IX

Coagulation factor IX (FIX; also known as Christmas factor or plasma thromboplastin component or PTC) is encoded by factor 9 (F9) and is a 415-amino acid serine protease synthesized in the liver. It is a vitamin K-dependent plasma protein that participates in the intrinsic pathway of blood coagulation by converting factor X to its active form in the presence of Ca²⁺ ions, phospholipids, and factor VIIIa. The plasma concentration of FIX is about 50 times that of factor VIII, and FIX has a half-life of about 24 hours.

The FIX expressed from the compositions and methods disclosed herein can be any wild type or variant FIX. In one example, the FIX is a human FIX protein. Human FIX is assigned UniProt reference number P00740. An exemplary amino acid sequence for human Factor IX is assigned NCBI Accession No. NP_000124.1 and is set forth in SEQ ID NO: 57. An exemplary human F9 mRNA (cDNA) sequence is assigned NCBI Accession No. NM_000133.4 and is set forth in SEQ ID NO: 58. An exemplary human F9 coding sequence is assigned CCDS ID CCDS14666.1 and is set forth in SEQ ID NO: 59.

In some examples, the FIX (e.g., human FIX) is a wild type FIX (e.g., wild type human FIX) sequence or a fragment thereof. For example, the FIX can be a fragment comprising the mature FIX amino acid sequence (i.e., the FIX sequence after removal of the signal peptide and propeptide), or a fragment comprising the mature FIX amino acid sequence and a portion of the propeptide. In a specific example, the FIX can comprise SEQ ID NO: 63 or can be at least 90%, at least 91%, at least 92%, at least 93%, at least 94%, at least 95%, at least 96%, at least 97%, at least 98%, at least 99%, or at least 99.5% identical to SEQ ID NO: 63.

In some examples, the FIX (e.g., human FIX) is not a hyperactive or hyperfunctional variant of FIX (i.e., the FIX does not have one or more mutations that increase the activity of the variant FIX relative to wild type). In other examples, the FIX (e.g., human FIX) is not a FVIII-independent variant of FIX (i.e., the FIX does not have one or more mutations that allow the variant FIX to activate coagulation in the absence of its cofactor, factor VIII). In other examples, the FIX (e.g., human FIX) is not a hyperactive or hyperfunctional variant of FIX and is not a FVIII-independent variant of FIX.

In other examples, the FIX (e.g., human FIX) is a variant FIX (e.g., a variant human FIX) or a fragment thereof. For example, the variant FIX or fragment thereof can comprise one or more mutations. In one example, the variant FIX or fragment thereof can have one or more mutations that increase the activity of the variant FIX (hyperactive or hyperfunctional) relative to wild type, such as an amino acid substitution in position R338 (e.g., R338A or R338L) and/or an amino acid substitution at position S377 (e.g., S377W). See, e.g., US 2019/0017039 and US 2020/0172892, each of which is herein incorporated by reference in its entirety for all purposes. The numbering referred to herein is the standard FIX numbering, with position 1 being the tyrosine at amino acid 47 in SEQ ID NO: 57 (i.e., the first amino acid of the mature FIX protein following the signal peptide and propeptide in SEQ ID NO: 57). Further examples of variant FIX comprise an amino acid at residue 338 chosen from alanine, leucine, valine, isoleucine, phenylalanine, tryptophan, methionine, serine, and threonine. Further FIX variants comprise an amino acid at residue 338 chosen from leucine, cysteine, aspartic acid, glutamic acid, histidine, lysine, asparagine, glutamine, or tyrosine. In another example, the variant FIX or fragment thereof can have one or more mutations that allow the variant FIX to activate coagulation in the absence of its cofactor, factor VIII, such as an amino acid substitution at position L6, V181, E185, Y259, A261, K265, Y345, I383, E388, or a combination thereof (e.g., L6F, V181I, E185D, E185S, Y259F, A261K, K265A, K265T, Y345F, I383V, E188G, or a combination thereof). See, e.g., U.S. Pat. Nos. 10,125,357, 10,000,748, 10,604,749, US 2008/0214462, U.S. Pat. Nos. 8,022,187, and 8,513,386, each of which is herein incorporated by reference in its entirety for all purposes. In another example, the variant FIX or fragment thereof can have one or more mutations that allow the variant FIX to activate coagulation in the absence of its cofactor, factor VIII, such as an amino acid substitution at position V181, K265, I383, or a combination thereof or at position L6, V181, K265, I383, E185, or a combination thereof (e.g., an L6F mutation, a V181I mutation, a K265A or K265T mutation, an I383V mutation, an E185D mutation, or a combination thereof such as L6F/V181I/K265A/I383V, L6F/V181I/K265T/I383V, V181I/K265A/1383V/E185D, V181I/K265T/1383V/E185D, V181I/K265A/1383V/E185S, or V181I/K265T/I383V/E185S, or a V181I mutation, a K265A or K265T mutation, an I383V mutation, or a combination thereof such as V181I/K265A/I383V or V181I/K265T/I383V). In another example, the variant FIX or fragment thereof can have one or more mutations that increase the activity of the variant FIX relative to wild type and one or more mutations that allow the variant FIX to activate coagulation in the absence of its cofactor, factor VIII.

The FIX coding sequences in the constructs disclosed herein may include wild type FIX coding sequences without any modifications. The FIX coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a construct disclosed herein has one or more cryptic splice sites mutated or removed. In another example, a FIX coding sequence in a construct disclosed herein has all identified cryptic splice sites mutated or removed. In another example, a FIX coding sequence in a construct disclosed herein has one or more CpG dinucleotides removed (i.e., is CpG depleted). In another example, a FIX coding sequence in a construct disclosed herein has all but one CpG dinucleotides removed. In another example, a FIX coding sequence in a construct disclosed herein has all CpG dinucleotides removed (i.e., is fully CpG depleted). In another example, a FIX coding sequence in a construct disclosed herein is codon optimized (e.g., codon optimized for expression in a human or mammal). In a specific example, a FIX coding sequence in a construct disclosed herein has one or more CpG dinucleotides removed (i.e., is CpG depleted) and has one or more cryptic splice sites mutated or removed. In another specific example, a FIX coding sequence in a construct disclosed herein has all but one CpG dinucleotides removed (e.g., introducing one CpG to mutate a cryptic splice site) and has one or more or all identified cryptic splice sites mutated or removed. In another specific example, a FIX coding sequence in a construct disclosed herein has one or more CpG dinucleotides removed (i.e., is CpG depleted) and is codon optimized (e.g., codon optimized for expression in a human or mammal). In another specific example, a FIX coding sequence in a construct disclosed herein has all CpG dinucleotides removed (i.e., is fully CpG depleted) and is codon optimized (e.g., codon optimized for expression in a human or mammal).

Various codon optimized FIX coding sequences are provided. See, e.g., WO 2023/077012 and US 2023-0149563, each of which is herein incorporated by reference in its entirety for all purposes.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 63. In another example, the FIX coding sequence is (or comprises a sequence) at least 95%, at least 96%, at least 97%, at least 98%, at least 99%, at least 99.5%, or 100% identical to SEQ ID NO: 61. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 63. In another example, the FIX coding sequence is (or comprises a sequence) at least 99%, at least 99.5%, or 100% identical to SEQ ID NO: 61. In another example, the FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the FIX coding sequence is (or comprises a sequence) at least 99%, at least 99.5%, or 100% identical to SEQ ID NO: 61 and encodes a FIX protein comprising the sequence set forth in SEQ ID NO: 63. In another example, the FIX coding sequence comprises the sequence set forth in SEQ ID NO: 61. In another example, the FIX coding sequence consists essentially of the sequence set forth in SEQ ID NO: 61. In another example, the FIX coding sequence consists of the sequence set forth in SEQ ID NO: 61. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 63 (and, e.g., retaining the activity of native FIX). Optionally, the FIX coding sequence in the above examples encodes a FIX protein comprising the sequence set forth in SEQ ID NO: 63. Optionally, the FIX coding sequence in the above examples encodes a FIX protein consisting essentially of the sequence set forth in SEQ ID NO: 63. Optionally, the FIX coding sequence in the above examples encodes a FIX protein consisting of the sequence set forth in SEQ ID NO: 63.

Various native and optimized native FIX coding sequences are also provided.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the FIX coding sequence is (or comprises a sequence) at least 95%, at least 96%, at least 97%, at least 98%, at least 99%, at least 99.5%, or 100% identical to SEQ ID NO: 60. In another example, the FIX coding sequence is (or comprises a sequence) at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the FIX coding sequence is (or comprises a sequence) at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the FIX coding sequence is (or comprises a sequence) at least 99%, at least 99.5%, or 100% identical to SEQ ID NO: 60. In another example, the FIX coding sequence is (or comprises a sequence) at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the FIX coding sequence is (or comprises a sequence) at least 99%, at least 99.5%, or 100% identical to SEQ ID NO: 60 and encodes a FIX protein comprising the sequence set forth in SEQ ID NO: 63. In another example, the FIX coding sequence comprises the sequence set forth in SEQ ID NO: 60. In another example, the FIX coding sequence consists essentially of the sequence set forth in SEQ ID NO: 60. In another example, the FIX coding sequence consists of the sequence set forth in SEQ ID NO: 60. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 63 (and, e.g., retaining the activity of native FIX). Optionally, the FIX coding sequence in the above examples encodes a FIX protein comprising the sequence set forth in SEQ ID NO: 63. Optionally, the FIX coding sequence in the above examples encodes a FIX protein consisting essentially of the sequence set forth in SEQ ID NO: 63. Optionally, the FIX coding sequence in the above examples encodes a FIX protein consisting of the sequence set forth in SEQ ID NO: 63.

When specific F9 nucleic acid constructs sequences are disclosed herein, they are meant to encompass the sequence disclosed or the reverse complement of the sequence. For example, if a F9 nucleic acid construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. Likewise, when unidirectional construct elements are disclosed herein in a specific 5′ to 3′ order, they are also meant to encompass the reverse complement of the order of those elements. One reason for this is that, in many embodiments disclosed herein, the F9 nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (2) Lysosomal Alpha-Glucosidase (GAA)

Lysosomal alpha-glucosidase (GAA; also known as acid alpha-glucosidase, acid alpha-glucosidase preproprotein, acid maltase, aglucosidase alfa, alpha-1,4-glucosidase, amyloglucosidase, glucoamylase, LYAG) is encoded by GAA. This enzyme is active in lysosomes, where it breaks down glycogen into glucose.

The human GAA gene (NCBI GeneID 2548) encodes a 952 amino acid protein. In the lysosome, human GAA is sequentially processed by proteases to polypeptides of 76-, 19.4-, and 3.9-kDa that remain associated. Further cleavage between R(200) and A(204) inefficiently converts the 76-kDa polypeptide to the mature 70-kDa form with an additional 10.4-kDa polypeptide. GAA maturation increases its affinity for glycogen by 7-10 fold. A signal peptide is encoded by amino acids 1-27, a propeptide encoded by amino acids 28-69, lysosomal alpha-glucosidase after removal of the signal peptide and propeptide is encoded by amino acids 70-952, the 76 kDa lysosomal alpha-glucosidase is encoded by amino acids 123-952, and the 70 kDa lysosomal alpha-glucosidase is encoded by amino acids 204-952.

The GAA expressed from the compositions and methods disclosed herein can be any wild type or variant GAA. In one example, the GAA is a human GAA protein. Human GAA is assigned UniProt reference number P10253. An exemplary amino acid sequence for human GAA is assigned NCBI Accession No. NP_000143.2 and is set forth in SEQ ID NO: 114. An exemplary human GAA mRNA (cDNA) sequence is assigned NCBI Accession No. NM_000152.5 and is set forth in SEQ ID NO: 115. An exemplary human GAA coding sequence is assigned CCDS ID CCDS32760.1 and is set forth in SEQ ID NO: 116. An exemplary mature human GAA amino acid sequence (i.e., the human GAA sequence after removal of the signal peptide and propeptide) starting at amino acid 70 (i.e., GAA 70-952) is set forth in SEQ ID NO: 117. An exemplary coding sequence for GAA 70-952 is set forth in SEQ ID NO: 118.

In some examples, the GAA (e.g., human GAA) is a wild type GAA (e.g., wild type human GAA) sequence or a fragment thereof. For example, the GAA can be a fragment comprising the mature GAA amino acid sequence (i.e., the GAA sequence after removal of the signal peptide and propeptide), a fragment comprising the 77 kDa form of GAA, or a fragment comprising the 70 kDa form of GAA. In a specific example, the GAA can comprise SEQ ID NO: 117 or can be at least 90%, at least 91%, at least 92%, at least 93%, at least 94%, at least 95%, at least 96%, at least 97%, at least 98%, at least 99%, or at least 99.5% identical to SEQ ID NO: 117. In another specific example, the GAA can consist essentially of SEQ ID NO: 117. In another specific example, the GAA can consist of SEQ ID NO: 117.

The GAA coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a GAA coding sequence in a construct disclosed herein has one or more cryptic splice sites mutated or removed. In some embodiments, the nucleotide at position 1095 (or the corresponding position when the GAA coding sequence is aligned with SEQ ID NO: 127) is a “G.” In some embodiments, the nucleotide at position 1098 (or the corresponding position when the GAA coding sequence is aligned with SEQ ID NO: 127) is a “C.” In some embodiments, the nucleotide at position 2343 (or the corresponding position when the GAA coding sequence is aligned with SEQ ID NO: 127) is a “G.” In some embodiments, the nucleotide at position 1095 (or the corresponding position when the GAA coding sequence is aligned with SEQ ID NO: 127) is a “G,” the nucleotide at position 1098 (or the corresponding position when the GAA coding sequence is aligned with SEQ ID NO: 127) is a “C,” and the nucleotide at position 2343 (or the corresponding position when the GAA coding sequence is aligned with SEQ ID NO: 127) is a “G.” In another example, a GAA coding sequence in a construct disclosed herein has all identified cryptic splice sites mutated or removed. In another example, a GAA coding sequence in a construct disclosed herein has one or more CpG dinucleotides removed (i.e., is CpG depleted). In another example, a GAA coding sequence in a construct disclosed herein has all CpG dinucleotides removed (i.e., is fully CpG depleted). In another example, a GAA coding sequence in a construct disclosed herein is codon optimized (e.g., codon optimized for expression in a human or mammal). In a specific example, a GAA coding sequence in a construct disclosed herein has one or more CpG dinucleotides removed (i.e., is CpG depleted) and has one or more cryptic splice sites mutated or removed. In another specific example, a GAA coding sequence in a construct disclosed herein has all CpG dinucleotides removed and has one or more or all identified cryptic splice sites mutated or removed. In another specific example, a GAA coding sequence in a construct disclosed herein has one or more CpG dinucleotides removed (i.e., is CpG depleted) and is codon optimized (e.g., codon optimized for expression in a human or mammal). In another specific example, a GAA coding sequence in a construct disclosed herein has all CpG dinucleotides removed (i.e., is fully CpG depleted) and is codon optimized (e.g., codon optimized for expression in a human or mammal).

Various codon optimized GAA coding sequences are provided. See, e.g., US 2025-0041455, WO 2025/029657, US 2023-0338477, and WO 2023/150623, each of which is herein incorporated by reference in its entirety for all purposes.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 127. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 127 and encodes a GAA protein (or a GAA protein comprising a sequence) at least 99%, at least 99.5%, or 100% identical to SEQ ID NO: 117. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 127 and encodes a GAA protein comprising the sequence set forth in SEQ ID NO: 117. In another example, the GAA coding sequence is (or comprises a sequence) at least 95%, at least 96%, at least 97%, at least 98%, at least 99%, at least 99.5%, or 100% identical to SEQ ID NO: 127. In another example, the GAA coding sequence is (or comprises a sequence) at least 95%, at least 96%, at least 97%, at least 98%, at least 99%, at least 99.5%, or 100% identical to SEQ ID NO: 127 and encodes a GAA protein (or a GAA protein comprising a sequence) at least 99%, at least 99.5%, or 100% identical to SEQ ID NO: 117. In another example, the GAA coding sequence is (or comprises a sequence) at least 95%, at least 96%, at least 97%, at least 98%, at least 99%, at least 99.5%, or 100% identical to SEQ ID NO: 127 and encodes a GAA protein comprising the sequence set forth in SEQ ID NO: 117. In another example, the GAA coding sequence is (or comprises a sequence) at least 99%, at least 99.5%, or 100% identical to SEQ ID NO: 127. In another example, the GAA coding sequence is (or comprises a sequence) at least 99%, at least 99.5%, or 100% identical to SEQ ID NO: 127 and encodes a GAA protein (or a GAA protein comprising a sequence) at least 99%, at least 99.5%, or 100% identical to SEQ ID NO: 117. In another example, the GAA coding sequence is (or comprises a sequence) at least 99%, at least 99.5%, or 100% identical to SEQ ID NO: 127 and encodes a GAA protein comprising the sequence set forth in SEQ ID NO: 117. In another example, the GAA coding sequence comprises the sequence set forth in SEQ ID NO: 127. In another example, the GAA coding sequence consists essentially of the sequence set forth in SEQ ID NO: 127. In another example, the GAA coding sequence consists of the sequence set forth in SEQ ID NO: 127. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 117 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 117 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 117 (and, e.g., retaining the activity of native GAA). Optionally, the GAA coding sequence in the above examples encodes a GAA protein comprising the sequence set forth in SEQ ID NO: 117. Optionally, the GAA coding sequence in the above examples encodes a GAA protein consisting essentially of the sequence set forth in SEQ ID NO: 117. Optionally, the GAA coding sequence in the above examples encodes a GAA protein consisting of the sequence set forth in SEQ ID NO: 117. In some embodiments, the nucleotide at position 1095 (or the corresponding position when the GAA coding sequence is aligned with SEQ ID NO: 127) is a “G.” In some embodiments, the nucleotide at position 1098 (or the corresponding position when the GAA coding sequence is aligned with SEQ ID NO: 127) is a “C.” In some embodiments, the nucleotide at position 2343 (or the corresponding position when the GAA coding sequence is aligned with SEQ ID NO: 127) is a “G.” In some embodiments, the nucleotide at position 1095 (or the corresponding position when the GAA coding sequence is aligned with SEQ ID NO: 127) is a “G,” the nucleotide at position 1098 (or the corresponding position when the GAA coding sequence is aligned with SEQ ID NO: 127) is a “C,” and the nucleotide at position 2343 (or the corresponding position when the GAA coding sequence is aligned with SEQ ID NO: 127) is a “G.”

When specific GAA or multidomain therapeutic protein nucleic acid constructs sequences are disclosed herein, they are meant to encompass the sequence disclosed or the reverse complement of the sequence. For example, if a GAA or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the GAA or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (3) CD63-Binding Delivery Domain

The multidomain therapeutic proteins disclosed herein can comprise a CD63-binding delivery domain fused to a GAA. See, e.g., US 2023-0338477, WO 2023/150623, US 2025-0041455, and WO 2025/029657, each of which is herein incorporated by reference in its entirety for all purposes. The CD63-binding domain provides binding to the internalization factor CD63 (UniProt Ref. P08962-1). CD63 (also known as CD63 antigen, granulophysin, lysosomal-associated membrane protein 3, LAMP-3, lysosome integral membrane protein 1, Limp1, melanoma-associated antigen ME491, OMA81H, ocular melanoma-associated antigen, tetraspanin-30, or Tspan-30) is a member of the tetraspanin superfamily of cell surface proteins that span the cell membrane four times. It is encoded by the CD63 gene (also known as MLA1 or TSPAN30). CD63 is expressed in virtually all tissues and is thought to be involved in forming and stabilizing signaling complexes. CD63 localizes to the cell membrane, lysosomal membrane, and late endosomal membrane. CD63 is known to associate with integrins and may be involved in epithelial-mesenchymal transitioning.

In some multidomain therapeutic proteins, the CD63-binding delivery domain is an antibody, an antibody fragment or other antigen-binding protein. In some multidomain therapeutic proteins, the CD63-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain. Examples of CD63-binding delivery domains can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes.

In a particular multidomain therapeutic protein, the CD63-binding delivery domain is an anti-CD63 scFv. In a specific example, the anti-CD63 scFv can comprise SEQ ID NO: 119 or can be at least 90%, at least 91%, at least 92%, at least 93%, at least 94%, at least 95%, at least 96%, at least 97%, at least 98%, at least 99%, or at least 99.5% identical to SEQ ID NO: 119. In another specific example, the anti-CD63 scFv can consist essentially of SEQ ID NO: 119. In another specific example, the anti-CD63 scFv can consist of SEQ ID NO: 119.

The CD63-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a CD63-binding delivery domain coding sequence in a construct disclosed herein has one or more cryptic splice sites mutated or removed. In another example, a CD63-binding delivery domain coding sequence in a construct disclosed herein has all identified cryptic splice sites mutated or removed. In another example, a CD63-binding delivery domain coding sequence in a construct disclosed herein has one or more CpG dinucleotides removed (i.e., is CpG depleted). In another example, a CD63-binding delivery domain coding sequence in a construct disclosed herein has all CpG dinucleotides removed (i.e., is fully CpG depleted). In another example, a CD63-binding delivery domain coding sequence in a construct disclosed herein is codon optimized (e.g., codon optimized for expression in a human or mammal). In a specific example, a CD63-binding delivery domain coding sequence in a construct disclosed herein has one or more CpG dinucleotides removed (i.e., is CpG depleted) and has one or more cryptic splice sites mutated or removed. In another specific example, a CD63-binding delivery domain coding sequence in a construct disclosed herein has all CpG dinucleotides removed and has one or more or all identified cryptic splice sites mutated or removed. In another specific example, a CD63-binding delivery domain coding sequence in a construct disclosed herein has one or more CpG dinucleotides removed (i.e., is CpG depleted) and is codon optimized (e.g., codon optimized for expression in a human or mammal). In another specific example, a CD63-binding delivery domain coding sequence in a construct disclosed herein has all CpG dinucleotides removed (i.e., is fully CpG depleted) and is codon optimized (e.g., codon optimized for expression in a human or mammal).

Various anti-CD63 scFv coding sequences are provided. See, e.g., US 2025-0041455, WO 2025/029657, US 2023-0338477, and WO 2023/150623, each of which is herein incorporated by reference in its entirety for all purposes.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 129. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 129 and encodes an anti-CD63 scFv protein (or an anti-CD63 scFv protein comprising a sequence) at least 99%, at least 99.5%, or 100% identical to SEQ ID NO: 119. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 129 and encodes an anti-CD63 scFv protein comprising the sequence set forth in SEQ ID NO: 119. In another example, the anti-CD63 scFv coding sequence is (or comprises a sequence) at least 95%, at least 96%, at least 97%, at least 98%, at least 99%, at least 99.5%, or 100% identical to SEQ ID NO: 129. In another example, the anti-CD63 scFv coding sequence is (or comprises a sequence) at least 95%, at least 96%, at least 97%, at least 98%, at least 99%, at least 99.5%, or 100% identical to SEQ ID NO: 129 and encodes an anti-CD63 scFv protein (or an anti-CD63 scFv protein comprising a sequence) at least 99%, at least 99.5%, or 100% identical to SEQ ID NO: 119. In another example, the anti-CD63 scFv coding sequence is (or comprises a sequence) at least 95%, at least 96%, at least 97%, at least 98%, at least 99%, at least 99.5%, or 100% identical to SEQ ID NO: 129 and encodes an anti-CD63 scFv protein comprising the sequence set forth in SEQ ID NO: 119. In another example, the anti-CD63 scFv coding sequence is (or comprises a sequence) at least 99%, at least 99.5%, or 100% identical to SEQ ID NO: 129. In another example, the anti-CD63 scFv coding sequence is (or comprises a sequence) at least 99%, at least 99.5%, or 100% identical to SEQ ID NO: 129 and encodes an anti-CD63 scFv protein (or an anti-CD63 scFv protein comprising a sequence) at least 99%, at least 99.5%, or 100% identical to SEQ ID NO: 119. In another example, the anti-CD63 scFv coding sequence is (or comprises a sequence) at least 99%, at least 99.5%, or 100% identical to SEQ ID NO: 129 and encodes an anti-CD63 scFv protein comprising the sequence set forth in SEQ ID NO: 119. In another example, the anti-CD63 scFv coding sequence comprises the sequence set forth in SEQ ID NO: 129. In another example, the anti-CD63 scFv coding sequence consists essentially of the sequence set forth in SEQ ID NO: 129. In another example, the anti-CD63 scFv coding sequence consists of the sequence set forth in SEQ ID NO: 129. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 119 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 119 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 119 (and, e.g., retaining CD63-binding activity). Optionally, the anti-CD63 scFv coding sequence in the above examples encodes an anti-CD63 scFv protein comprising the sequence set forth in SEQ ID NO: 119. Optionally, the anti-CD63 scFv coding sequence in the above examples encodes an anti-CD63 scFv protein consisting essentially of the sequence set forth in SEQ ID NO: 119. Optionally, the anti-CD63 scFv coding sequence in the above examples encodes an anti-CD63 scFv protein consisting of the sequence set forth in SEQ ID NO: 119. In some embodiments, the nucleotide at position 3 (or the corresponding position when the anti-CD63 scFv coding sequence is aligned with SEQ ID NO: 129) is an “A.” In some embodiments, the nucleotide at position 132 (or the corresponding position when the anti-CD63 scFv coding sequence is aligned with SEQ ID NO: 129) is an “A.” In some embodiments, the nucleotide at position 273 (or the corresponding position when the anti-CD63 scFv coding sequence is aligned with SEQ ID NO: 129) is a “T.” In some embodiments, the nucleotide at position 3 (or the corresponding position when the anti-CD63 scFv coding sequence is aligned with SEQ ID NO: 129) is an “A,” the nucleotide at position 132 (or the corresponding position when the anti-CD63 scFv coding sequence is aligned with SEQ ID NO: 129) is an “A,” and the nucleotide at position 273 (or the corresponding position when the anti-CD63 scFv coding sequence is aligned with SEQ ID NO: 129) is a “T.”

When specific anti-CD63 scFv or multidomain therapeutic protein nucleic acid constructs sequences are disclosed herein, they are meant to encompass the sequence disclosed or the reverse complement of the sequence. For example, if an anti-CD63 scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-CD63 scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (4) TfR-Binding Delivery Domain

The multidomain therapeutic proteins disclosed herein can comprise a TfR-binding delivery domain fused to a GAA. See, e.g., US 2023-0338477, WO 2023/150623, US 2025-0041455, and WO 2025/029657, each of which is herein incorporated by reference in its entirety for all purposes. The TfR-binding domain provides binding to the internalization factor transferrin receptor protein 1 (TfR; UniProt Ref. P02786). TfR (also known as TR, TfR1, and Trfr) is encoded by the TFRC gene. TfR is expressed in muscle and on brain endothelial cells. Transcytosis of TfR in these cells enables blood-brain-barrier crossing. In some embodiments, the multidomain therapeutic proteins comprising a TfR-binding delivery domain (e.g., scFv) fused to a GAA do not alter transferrin uptake. In some embodiments, the multidomain therapeutic proteins comprising a TfR-binding delivery domain (e.g., scFv) fused to a GAA do not alter iron homeostasis. In some embodiments, the multidomain therapeutic proteins comprising a TfR-binding delivery domain (e.g., scFv) fused to a GAA do not alter transferrin uptake or iron homeostasis.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, at TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g., Accession numbers NP_001121620.1; BAD92491.1; and NP_001300894.1.; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 March; 35(3):292-6. Unless otherwise stated, transferrin receptor as used herein generally refers to transferrin receptor 1 (e.g., human transferrin receptor 1).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

Provided herein are antigen-binding proteins, such as antibodies, antigen-binding fragments thereof, such as Fabs and scFvs, that bind specifically to the transferrin receptor, preferably the human transferrin receptor 1 (anti-hTfR). For example, in an embodiment, the anti-hTfR is in the form of a fusion protein. The fusion protein includes the anti-hTfR antigen-binding protein fused to GAA. The anti-hTfRs efficiently cross the blood-brain barrier (BBB) and can, thereby, deliver the fused GAA to the brain.

In an embodiment, an anti-hTfR scFv:GAA fusion protein includes an scFv comprising the arrangement of variable regions as follows LCVR-HCVR or HCVR-LCVR, wherein the HCVR and LCVR are optionally connected by a linker and the scFv is connected, optionally by a linker, to GAA.

An anti-hTfR:GAA optionally comprises a signal peptide, connected to the antigen-binding protein that binds specifically to transferrin receptor (TfR), preferably, human transferrin receptor (hTfR) which is fused (optionally by a linker) to GAA. In an embodiment, the signal peptide is the mROR signal sequence. The term “fused” or “tethered” with regard to fused polypeptides refers to polypeptides joined directly or indirectly (e.g., via a linker or other polypeptide).

Various anti-hTfR antibody sequences are provided, e.g., in US 2025-0041455, WO 2025/029657, US 2023-0338477, and WO 2023/150623, each of which is herein incorporated by reference in its entirety for all purposes.

In some multidomain therapeutic proteins, the TfR-binding delivery domain is an antibody, an antibody fragment or other antigen-binding protein. In some multidomain therapeutic proteins, the TfR-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain.

The TfR-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a TfR-binding delivery domain coding sequence in a construct disclosed herein has one or more cryptic splice sites mutated or removed. In another example, a TfR-binding delivery domain coding sequence in a construct disclosed herein has all identified cryptic splice sites mutated or removed. In another example, a TfR-binding delivery domain coding sequence in a construct disclosed herein has one or more CpG dinucleotides removed (i.e., is CpG depleted). In another example, a TfR-binding delivery domain coding sequence in a construct disclosed herein has all CpG dinucleotides removed (i.e., is fully CpG depleted). In another example, a TfR-binding delivery domain coding sequence in a construct disclosed herein is codon optimized (e.g., codon optimized for expression in a human or mammal). In a specific example, a CDTfR63-binding delivery domain coding sequence in a construct disclosed herein has one or more CpG dinucleotides removed (i.e., is CpG depleted) and has one or more cryptic splice sites mutated or removed. In another specific example, a TfR-binding delivery domain coding sequence in a construct disclosed herein has all CpG dinucleotides removed and has one or more or all identified cryptic splice sites mutated or removed. In another specific example, a TfR-binding delivery domain coding sequence in a construct disclosed herein has one or more CpG dinucleotides removed (i.e., is CpG depleted) and is codon optimized (e.g., codon optimized for expression in a human or mammal). In another specific example, a TfR-binding delivery domain coding sequence in a construct disclosed herein has all CpG dinucleotides removed (i.e., is fully CpG depleted) and is codon optimized (e.g., codon optimized for expression in a human or mammal).

Various anti-TfR scFv coding sequences are provided. See, e.g., US 2025-0041455, WO 2025/029657, US 2023-0338477, and WO 2023/150623, each of which is herein incorporated by reference in its entirety for all purposes.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 122. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 122 and encodes an anti-TfR scFv protein (or an anti-TfR scFv protein comprising a sequence) at least 99%, at least 99.5%, or 100% identical to SEQ ID NO: 121. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 122 and encodes an anti-TfR scFv protein comprising the sequence set forth in SEQ ID NO: 121. In another example, the anti-TfR scFv coding sequence is (or comprises a sequence) at least 95%, at least 96%, at least 97%, at least 98%, at least 99%, at least 99.5%, or 100% identical to SEQ ID NO: 122. In another example, the anti-TfR scFv coding sequence is (or comprises a sequence) at least 95%, at least 96%, at least 97%, at least 98%, at least 99%, at least 99.5%, or 100% identical to SEQ ID NO: 122 and encodes an anti-TfR scFv protein (or an anti-TfR scFv protein comprising a sequence) at least 99%, at least 99.5%, or 100% identical to SEQ ID NO: 121. In another example, the anti-TfR scFv coding sequence is (or comprises a sequence) at least 95%, at least 96%, at least 97%, at least 98%, at least 99%, at least 99.5%, or 100% identical to SEQ ID NO: 122 and encodes an anti-TfR scFv protein comprising the sequence set forth in SEQ ID NO: 121. In another example, the anti-TfR scFv coding sequence is (or comprises a sequence) at least 99%, at least 99.5%, or 100% identical to SEQ ID NO: 122. In another example, the anti-TfR scFv coding sequence is (or comprises a sequence) at least 99%, at least 99.5%, or 100% identical to SEQ ID NO: 122 and encodes an anti-TfR scFv protein (or an anti-TfR scFv protein comprising a sequence) at least 99%, at least 99.5%, or 100% identical to SEQ ID NO: 121. In another example, the anti-TfR scFv coding sequence is (or comprises a sequence) at least 99%, at least 99.5%, or 100% identical to SEQ ID NO: 122 and encodes an anti-TfR scFv protein comprising the sequence set forth in SEQ ID NO: 121. In another example, the anti-TfR scFv coding sequence comprises the sequence set forth in SEQ ID NO: 122. In another example, the anti-TfR scFv coding sequence consists essentially of the sequence set forth in SEQ ID NO: 122. In another example, the anti-TfR scFv coding sequence consists of the sequence set forth in SEQ ID NO: 122. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 121 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 121 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 121 (and, e.g., retaining TfR-binding activity). Optionally, the anti-TfR scFv coding sequence in the above examples encodes an anti-TfR scFv protein comprising the sequence set forth in SEQ ID NO: 121. Optionally, the anti-TfR scFv coding sequence in the above examples encodes an anti-TfR scFv protein consisting essentially of the sequence set forth in SEQ ID NO: 121. Optionally, the anti-TfR scFv coding sequence in the above examples encodes an anti-TfR scFv protein consisting of the sequence set forth in SEQ ID NO: 121.

When specific anti-TfR scFv or multidomain therapeutic protein nucleic acid constructs sequences are disclosed herein, they are meant to encompass the sequence disclosed or the reverse complement of the sequence. For example, if an anti-TfR scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-TfR scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (5) Bidirectional Constructs

The nucleic acid constructs disclosed herein can be bidirectional constructs. Such bidirectional constructs can allow for enhanced insertion and expression of encoded polypeptide of interest. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus or a cleavage site or target insertion site, allowing the expression of the polypeptide of interest when inserted in either orientation, thereby enhancing expression efficiency.

A bidirectional construct as disclosed herein can comprise at least two nucleic acid segments, wherein a first segment comprises a first coding sequence for the polypeptide of interest, and a second segment comprises the reverse complement of a second coding sequence for the polypeptide of interest, or vice versa. However, other bidirectional constructs disclosed herein can comprise at least two nucleic acid segments, wherein the first segment comprises a coding sequence for a polypeptide of interest, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the polypeptide of interest or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

Examples of bidirectional constructs and elements and features of unidirectional constructs are provided, e.g., in US 2023-0149563, WO 2023/077012, US 2025-0041455, and WO 2025/029657, each of which are herein incorporated by reference in its entirety for all purposes.

In an exemplary bidirectional construct, the second segment is located 3′ of the first segment, the first polypeptide of interest coding sequence and the second polypeptide of interest coding sequence both encode the same human polypeptide of interest, the second polypeptide of interest coding sequence adopts a different codon usage from the codon usage of the first polypeptide of interest coding sequence, the first segment comprises a first polyadenylation signal sequence located 3′ of the first polypeptide of interest coding sequence, the second segment comprises a reverse complement of a second polyadenylation signal sequence located 5′ of the reverse complement of the second polypeptide of interest coding sequence, the first segment comprises a first splice acceptor site located 5′ of the first polypeptide of interest coding sequence, the second segment comprises a reverse complement of a second splice acceptor site located 3′ of the reverse complement of the second polypeptide of interest coding sequence, the nucleic acid construct does not comprise a promoter that drives expression of the first polypeptide of interest or the second polypeptide of interest, and optionally the nucleic acid construct does not comprise a homology arm.

### (6) Unidirectional Constructs

The nucleic acid constructs disclosed herein can be unidirectional constructs. When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

Examples of unidirectional constructs and elements and features of unidirectional constructs are provided, e.g., in US 2025-0041455 and WO 2025/029657, each of which are herein incorporated by reference in its entirety for all purposes.

In an exemplary unidirectional construct, the construct comprises a polyadenylation signal sequence located 3′ of the coding sequence for the polypeptide of interest, the construct comprises a splice acceptor site located 5′ of the coding sequence for the polypeptide of interest, and the nucleic acid construct does not comprise a promoter that drives expression of the polypeptide of interest, and optionally the nucleic acid construct does not comprise a homology arm.

### (7) F9 Nucleic Acid Constructs

The F9 nucleic acid constructs disclosed herein can be bidirectional constructs. Such bidirectional constructs can allow for enhanced insertion and expression of encoded FIX. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus, allowing the expression of FIX when inserted in either orientation, thereby enhancing expression efficiency, as exemplified herein. For example, when used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a cleavage site or target insertion site, allowing the expression of FIX when inserted in either orientation, thereby enhancing insertion and expression efficiency, as exemplified herein.

A bidirectional construct as disclosed herein can comprise at least two nucleic acid segments, wherein a first segment comprises a first FIX coding sequence, and a second segment comprises the reverse complement of a second FIX coding sequence, or vice versa. However, other bidirectional constructs disclosed herein can comprise at least two nucleic acid segments, wherein the first segment comprises a FIX coding sequence, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the FIX or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode FIX, the at least two segments can encode the same FIX protein or different FIX proteins. The different FIX proteins can be at least about 90%, at least about 95%, at least about 96%, at least about 97%, at least about 98%, at least about 99%, or at least about 99.5% identical. For example, the first segment can encode a wild type FIX protein or fragment thereof, and the second segment can encode a variant FIX protein or fragment thereof, or vice versa. Alternatively, the first segment can encode a first variant FIX protein, and the second segment can encode a second variant FIX protein that is different from the first variant FIX protein. Preferably, the two segments encode the same FIX protein (i.e., 100% identical).

Even when the two segments encode the same FIX protein, the FIX coding sequence in the first segment can differ from the FIX coding sequence in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second FIX coding sequence can be codon optimized or may use one or more alternative codons for one or more amino acids of the same FIX (i.e., same amino acid sequence) encoded by the FIX coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a FIX coding sequence that adopts different codon usage from that of the FIX coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of FIX.

In some cases, the bidirectional nucleic acid construct does not comprise a promoter that drives the expression of FIX. For example, the expression of FIX can be driven by a promoter of the host cell (e.g., the endogenous ALB promoter when the transgene is integrated into a host cell's ALB locus). In other cases, the bidirectional nucleic acid construct can comprise one or more promoters operably linked to the FIX coding sequences. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first FIX coding sequence and/or the reverse complement of a promoter that drives expression of the reverse complement of the second FIX coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a heterologous FIX.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a CpG-depleted BGH polyadenylation signal. Exemplary polyadenylation signal sequences are provided in US 2023-0149563 and WO 2023/077012, each of which is herein incorporated by reference in its entirety for all purposes.

In some bidirectional constructs, both the first segment and the second segment comprise a polyadenylation tail sequence.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. Exemplary splice acceptor sequences are provided in US 2023-0149563 and WO 2023/077012, each of which is herein incorporated by reference in its entirety for all purposes.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The FIX coding sequences in the bidirectional constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a bidirectional construct disclosed herein has one or more cryptic splice sites mutated or removed. In another example, a FIX coding sequence in a bidirectional construct disclosed herein has all identified cryptic splice sites mutated or removed. In another example, a FIX coding sequence in a bidirectional construct disclosed herein has one or more CpG dinucleotides removed (i.e., is CpG depleted). In another example, a FIX coding sequence in a bidirectional construct disclosed herein has all but one CpG dinucleotides removed. In another example, a FIX coding sequence in a bidirectional construct disclosed herein has all CpG dinucleotides removed (i.e., is fully CpG depleted). In another example, a FIX coding sequence in a bidirectional construct disclosed herein is codon optimized (e.g., codon optimized for expression in a human or mammal). In a specific example, a FIX coding sequence in a bidirectional construct disclosed herein has one or more CpG dinucleotides removed (i.e., is CpG depleted) and has one or more cryptic splice sites mutated or removed. In another specific example, a FIX coding sequence in a bidirectional construct disclosed herein has all but one CpG dinucleotides removed and has one or more or all identified cryptic splice sites mutated or removed. In another specific example, a FIX coding sequence in a bidirectional construct disclosed herein has one or more CpG dinucleotides removed (i.e., is CpG depleted) and is codon optimized (e.g., codon optimized for expression in a human or mammal). In another specific example, a FIX coding sequence in a bidirectional construct disclosed herein has all CpG dinucleotides removed (i.e., is fully CpG depleted) and is codon optimized (e.g., codon optimized for expression in a human or mammal).

In one specific example, one FIX coding sequence in a bidirectional construct disclosed herein has one or more CpG dinucleotides removed (i.e., is CpG depleted) and has one or more cryptic splice sites mutated or removed, and the other FIX coding sequence in the bidirectional construct disclosed herein has one or more CpG dinucleotides removed (i.e., is CpG depleted) and is codon optimized (e.g., codon optimized for expression in a human or mammal). In another specific example, one FIX coding sequence in a bidirectional construct disclosed herein has all but one CpG dinucleotides removed and has one or more or all identified cryptic splice sites mutated or removed, and the other FIX coding sequence in the bidirectional construct disclosed herein has all CpG dinucleotides removed (i.e., is fully CpG depleted) and is codon optimized (e.g., codon optimized for expression in a human or mammal).

In an exemplary bidirectional construct, the second segment is located 3′ of the first segment, the first FIX coding sequence and the second FIX coding sequence both encode the same human FIX protein, the second FIX coding sequence adopts a different codon usage from the codon usage of the first FIX coding sequence, the first segment comprises a first polyadenylation signal sequence located 3′ of the first FIX coding sequence, the second segment comprises a reverse complement of a second polyadenylation signal sequence located 5′ of the reverse complement of the second FIX coding sequence, the first segment comprises a first splice acceptor site located 5′ of the first FIX coding sequence, the second segment comprises a reverse complement of a second splice acceptor site located 3′ of the reverse complement of the second FIX coding sequence, the nucleic acid construct does not comprise a promoter that drives expression of the first FIX protein or the second FIX protein, and optionally the nucleic acid construct does not comprise a homology arm.

In one example of a bidirectional construct, the first FIX protein coding sequence and the second FIX protein coding sequence are different but encode the same FIX protein sequence, and one of the FIX coding sequences is CpG-depleted (e.g., fully CpG-depleted) and/or codon optimized (e.g., CpG-depleted and codon optimized or fully CpG-depleted and codon optimized). Examples of FIX coding sequences and F9 nucleic acid constructs are provided, e.g., in US 2023-0149563 and WO 2023/077012, each of which is herein incorporated by reference in its entirety for all purposes.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 63. In another example, the one of the FIX coding sequences is (or comprises a sequence) at least 95%, at least 96%, at least 97%, at least 98%, at least 99%, at least 99.5%, or 100% identical to SEQ ID NO: 61. In another example, the one of the FIX coding sequences is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 63. In another example, the one of the FIX coding sequences is (or comprises a sequence) at least 99%, at least 99.5%, or 100% identical to SEQ ID NO: 61. In another example, the one of the FIX coding sequences is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 99%, at least 99.5%, or 100% identical to SEQ ID NO: 61 and encodes a FIX protein comprising the sequence set forth in SEQ ID NO: 63. In another example, the one of the FIX coding sequences comprises the sequence set forth in SEQ ID NO: 61. In another example, the one of the FIX coding sequences consists essentially of the sequence set forth in SEQ ID NO: 61. In another example, the one of the FIX coding sequences consists of the sequence set forth in SEQ ID NO: 61. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 63 (and, e.g., retaining the activity of native FIX). Optionally, the one of the FIX coding sequences in the above examples encodes a FIX protein comprising the sequence set forth in SEQ ID NO: 63. Optionally, the one of the FIX coding sequences in the above examples encodes a FIX protein consisting essentially of the sequence set forth in SEQ ID NO: 63. Optionally, the one of the FIX coding sequences in the above examples encodes a FIX protein consisting of the sequence set forth in SEQ ID NO: 63.

Various optimized native FIX coding sequences are also provided.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the one of the FIX coding sequences is (or comprises a sequence) at least 95%, at least 96%, at least 97%, at least 98%, at least 99%, at least 99.5%, or 100% identical to SEQ ID NO: 60. In another example, the one of the FIX coding sequences is (or comprises a sequence) at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the one of the FIX coding sequences is (or comprises a sequence) at least 99%, at least 99.5%, or 100% identical to SEQ ID NO: 60. In another example, the one of the FIX coding sequences is (or comprises a sequence) at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 99%, at least 99.5%, or 100% identical to SEQ ID NO: 60 and encodes a FIX protein comprising the sequence set forth in SEQ ID NO: 63. In another example, the one of the FIX coding sequences comprises the sequence set forth in SEQ ID NO: 60. In another example, the one of the FIX coding sequences consists essentially of the sequence set forth in SEQ ID NO: 60. In another example, the one of the FIX coding sequences consists of the sequence set forth in SEQ ID NO: 60. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 63 (and, e.g., retaining the activity of native FIX). Optionally, the one of the FIX coding sequences in the above examples encodes a FIX protein comprising the sequence set forth in SEQ ID NO: 63. Optionally, the FIX coding sequence in the above examples encodes a FIX protein consisting essentially of the sequence set forth in SEQ ID NO: 63. Optionally, the one of the FIX coding sequences in the above examples encodes a FIX protein consisting of the sequence set forth in SEQ ID NO: 63.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 63. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 63. In another example, the one of the FIX coding sequences comprises the sequence set forth in SEQ ID NO: 61. In another example, the one of the FIX coding sequences consists essentially of the sequence set forth in SEQ ID NO: 61. In another example, the one of the FIX coding sequences consists of the sequence set forth in SEQ ID NO: 61. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the other FIX coding sequence is (or comprises a sequence) at least 95%, at least 96%, at least 97%, at least 98%, at least 99%, at least 99.5%, or 100% identical to SEQ ID NO: 60. In another example, the other FIX coding sequence is (or comprises a sequence) at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the other FIX coding sequence is (or comprises a sequence) at least 99%, at least 99.5%, or 100% identical to SEQ ID NO: 60. In another example, the other FIX coding sequence is (or comprises a sequence) at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 99%, at least 99.5%, or 100% identical to SEQ ID NO: 60 and encodes a FIX protein comprising the sequence set forth in SEQ ID NO: 63. In another example, the other FIX coding sequence comprises the sequence set forth in SEQ ID NO: 60. In another example, the other FIX coding sequence consists essentially of the sequence set forth in SEQ ID NO: 60. In another example, the other FIX coding sequence consists of the sequence set forth in SEQ ID NO: 60. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 63 (and, e.g., retaining the activity of native FIX). Optionally, one or both of the FIX coding sequence in the above examples encodes a FIX protein comprising the sequence set forth in SEQ ID NO: 63. Optionally, one or both of the FIX coding sequence in the above examples encodes a FIX protein consisting essentially of the sequence set forth in SEQ ID NO: 63. Optionally, one or both of the FIX coding sequence in the above examples encodes a FIX protein consisting of the sequence set forth in SEQ ID NO: 63.

In another example, the one of the FIX coding sequences comprises the sequence set forth in SEQ ID NO: 61. In another example, the one of the FIX coding sequences consists essentially of the sequence set forth in SEQ ID NO: 61. In another example, the one of the FIX coding sequences consists of the sequence set forth in SEQ ID NO: 61. In another example, the other FIX coding sequence comprises the sequence set forth in SEQ ID NO: 60. In another example, the other FIX coding sequence consists essentially of the sequence set forth in SEQ ID NO: 60. In another example, the other FIX coding sequence consists of the sequence set forth in SEQ ID NO: 60. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence in the above examples encodes a FIX protein comprising the sequence set forth in SEQ ID NO: 63. Optionally, one or both of the FIX coding sequence in the above examples encodes a FIX protein consisting essentially of the sequence set forth in SEQ ID NO: 63. Optionally, one or both of the FIX coding sequence in the above examples encodes a FIX protein consisting of the sequence set forth in SEQ ID NO: 63.

In another example, the one of the FIX coding sequences consists of the sequence set forth in SEQ ID NO: 61. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the other FIX coding sequence is (or comprises a sequence) at least 95%, at least 96%, at least 97%, at least 98%, at least 99%, at least 99.5%, or 100% identical to SEQ ID NO: 60. In another example, the other FIX coding sequence is (or comprises a sequence) at least 95%, at least 96%, at least 97%, at least 98%, at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 95%, at least 96%, at least 97%, at least 98%, at least 99%, at least 99.5%, or 100% identical to SEQ ID NO: 60 and encodes a FIX protein comprising the sequence set forth in SEQ ID NO: 63. In another example, the other FIX coding sequence is (or comprises a sequence) at least 99%, at least 99.5%, or 100% identical to SEQ ID NO: 60. In another example, the other FIX coding sequence is (or comprises a sequence) at least 99%, at least 99.5%, or 100% identical to SEQ ID NO: 60 and encodes a FIX protein (or a FIX protein comprising a sequence) at least 99%, at least 99.5%, or 100% identical to SEQ ID NO: 63. In another example, the other FIX coding sequence is (or comprises a sequence) at least 99%, at least 99.5%, or 100% identical to SEQ ID NO: 60 and encodes a FIX protein comprising the sequence set forth in SEQ ID NO: 63. In another example, the other FIX coding sequence comprises the sequence set forth in SEQ ID NO: 60. In another example, the other FIX coding sequence consists essentially of the sequence set forth in SEQ ID NO: 60. In another example, the other FIX coding sequence consists of the sequence set forth in SEQ ID NO: 60. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 63 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 63 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 63 (and, e.g., retaining the activity of native FIX). Optionally, one or both of the FIX coding sequence in the above examples encodes a FIX protein comprising the sequence set forth in SEQ ID NO: 63. Optionally, one or both of the FIX coding sequence in the above examples encodes a FIX protein consisting essentially of the sequence set forth in SEQ ID NO: 63. Optionally, one or both of the FIX coding sequence in the above examples encodes a FIX protein consisting of the sequence set forth in SEQ ID NO: 63.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 64 or 62. In another particular example, an exemplary bidirectional construct comprises a sequence at least 95%, at least 96%, at least 97%, at least 98%, at least 99%, at least 99.5%, or 100% identical to SEQ ID NO: 64 or 62. In another particular example, an exemplary bidirectional construct comprises a sequence at least 99%, at least 99.5%, or 100% identical to SEQ ID NO: 64 or 62. In another particular example, an exemplary bidirectional construct comprises SEQ ID NO: 64 or 62. In another particular example, an exemplary bidirectional construct consists essentially of SEQ ID NO: 64 or 62. In another particular example, an exemplary bidirectional construct consists of SEQ ID NO: 64 or 62.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 64. In another particular example, an exemplary bidirectional construct comprises a sequence at least 95%, at least 96%, at least 97%, at least 98%, at least 99%, at least 99.5%, or 100% identical to SEQ ID NO: 64. In another particular example, an exemplary bidirectional construct comprises a sequence at least 99%, at least 99.5%, or 100% identical to SEQ ID NO: 64. In another particular example, an exemplary bidirectional construct comprises SEQ ID NO: 64. In another particular example, an exemplary bidirectional construct consists essentially of SEQ ID NO: 64. In another particular example, an exemplary bidirectional construct consists of SEQ ID NO: 64.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 62. In another particular example, an exemplary bidirectional construct comprises a sequence at least 95%, at least 96%, at least 97%, at least 98%, at least 99%, at least 99.5%, or 100% identical to SEQ ID NO: 62. In another particular example, an exemplary bidirectional construct comprises a sequence at least 99%, at least 99.5%, or 100% identical to SEQ ID NO: 62. In another particular example, an exemplary bidirectional construct comprises SEQ ID NO: 62. In another particular example, an exemplary bidirectional construct consists essentially of SEQ ID NO: 62. In another particular example, an exemplary bidirectional construct consists of SEQ ID NO: 62.

The F9 nucleic acid constructs disclosed herein can be unidirectional constructs. Exemplary unidirectional F9 constructs are described, e.g., in US 2023-0149563 and WO 2023/077012, each of which is herein incorporated by reference in its entirety for all purposes.

### (8) Multidomain Therapeutic Protein Nucleic Acid Constructs

The multidomain therapeutic protein nucleic acid constructs disclosed herein can be unidirectional constructs or bidirectional constructs. Examples of such constructs are provided in PCT/US2023/061858 and U.S. Ser. No. 18/163,698, each of which is herein incorporated by reference in its entirety for all purposes. When specific construct sequences are disclosed herein, they are meant to encompass the sequence disclosed or the reverse complement of the sequence. For example, if a construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the nucleic acid constructs, the multidomain therapeutic protein coding sequence, the CD63-binding delivery domain coding sequence, and/or the GAA coding sequence can be codon-optimized for expression in a host cell. For example, the multidomain therapeutic protein coding sequence, the CD63-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

In the nucleic acid constructs, the multidomain therapeutic protein coding sequence, the TfR-binding delivery domain coding sequence, and/or the GAA coding sequence can be codon-optimized for expression in a host cell. For example, the multidomain therapeutic protein coding sequence, the TfR-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The nucleic acid constructs disclosed herein can be modified to include any suitable structural feature as needed for any particular use and/or that confers one or more desired functions. For example, the nucleic acid constructs disclosed herein need not comprise a homology arm and/or can be, for example, homology-independent donor constructs.

In some cases, the nucleic acid construct does not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell's ALB locus). In other cases, the nucleic acid construct can comprise one or more promoters operably linked to the multidomain therapeutic protein coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some nucleic acid constructs can comprise a promoter that drives expression of the multidomain therapeutic protein. For example, the promoter may be a liver-specific promoter. Examples of liver-specific promoters include TTR promoters, such as human or mouse TTR promoters. In one example, the construct may comprise a TTR promoter, such as a mouse TTR promoter or a human TTR promoter (e.g., the coding sequence for the multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the multidomain therapeutic protein is operably linked to the SERPINA1 enhancer and the TTR promoter).

The nucleic acid constructs can, in some cases, comprise one or more polyadenylation tail sequences or polyadenylation signal sequences. Some nucleic acid constructs can comprise a polyadenylation signal sequence located 3′ of the multidomain therapeutic protein coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal.

In one example, the polyadenylation signal can comprise a BGH polyadenylation signal. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. In a specific example, the BGH polyadenylation signal can be upstream (5′) of the SV40 polyadenylation signal (e.g., unidirectional SV40 late polyadenylation signal). In another example, the polyadenylation signal can comprise a combination of a BGH polyadenylation signal and a synthetic polyadenylation signal. In some embodiments, the nucleic acid construct is a unidirectional construct. Exemplary polyadenylation signal sequences are provided in US 2025-0041455 and WO 2025/029657, each of which is herein incorporated by reference in its entirety for all purposes.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. Examples of stuffer sequences and MAZ elements are provided in US 2025-0041455 and WO 2025/029657, each of which is herein incorporated by reference in its entirety for all purposes.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal.

The nucleic acid constructs can, in some cases, comprise one or more splice acceptor sites. Some nucleic acid constructs comprise a splice acceptor site located 5′ of the multidomain therapeutic protein coding sequence. In a specific example, the splice acceptor is a mouse Alb exon 2 splice acceptor. Examples of splice acceptor sequences are provided in US 2025-0041455 and WO 2025/029657, each of which is herein incorporated by reference in its entirety for all purposes.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a GAA splice acceptor. For example, such a splice acceptor can be derived from the human GAA gene. Alternatively, such a splice acceptor can be derived from the mouse GAA gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The nucleic acid constructs can be circular or linear. For example, a nucleic acid construct can be linear. The nucleic acid constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the nucleic acid construct is single-stranded (e.g., single-stranded DNA).

The nucleic acid constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the nucleic acid constructs disclosed herein can comprise one, two, or three ITRs or can comprise no more than two ITRs. Various methods of structural modifications are known.

Similarly, one or both ends of the nucleic acid construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the nucleic acid constructs disclosed herein can be introduced into a cell as part of a vector having additional sequences such as, for example, replication origins, promoters, and genes encoding antibiotic resistance. The nucleic acid constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The multidomain therapeutic protein coding sequence, the CD63-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a CD63-binding delivery domain coding sequence, and/or a GAA coding sequence in a nucleic acid construct disclosed herein has one or more cryptic splice sites mutated or removed. In another example, a multidomain therapeutic protein coding sequence, a CD63-binding delivery domain coding sequence, and/or a GAA coding sequence in a nucleic acid construct disclosed herein has all identified cryptic splice sites mutated or removed. In another example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a CD63-binding delivery domain coding sequence, and/or a GAA coding sequence in a nucleic acid construct disclosed herein has all CpG dinucleotides removed. In another example, a multidomain therapeutic protein coding sequence, a CD63-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a CD63-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a CD63-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

The multidomain therapeutic protein coding sequence, the TfR-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a TfR-binding delivery domain coding sequence, and/or a GAA coding sequence in a nucleic acid construct disclosed herein has one or more cryptic splice sites mutated or removed. In another example, a multidomain therapeutic protein coding sequence, a TfR-binding delivery domain coding sequence, and/or a GAA coding sequence in a nucleic acid construct disclosed herein has all identified cryptic splice sites mutated or removed. In another example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a TfR-binding delivery domain coding sequence, and/or a GAA coding sequence in a nucleic acid construct disclosed herein has all CpG dinucleotides removed. In another example, a multidomain therapeutic protein coding sequence, a TfR-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a TfR-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a TfR-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

In an exemplary nucleic acid construct, the construct comprises a polyadenylation signal sequence located 3′ of the multidomain therapeutic protein coding sequence, the construct comprises a splice acceptor site located 5′ of the multidomain therapeutic protein coding sequence, and the nucleic acid construct does not comprise a promoter that drives expression of the multidomain therapeutic protein, and optionally the nucleic acid construct does not comprise a homology arm.

In a specific example of a multidomain therapeutic protein nucleic acid construct, the encoded multidomain therapeutic protein can comprise SEQ ID NO: 126 or can be at least 90%, at least 91%, at least 92%, at least 93%, at least 94%, at least 95%, at least 96%, at least 97%, at least 98%, at least 99%, or at least 99.5% identical to SEQ ID NO: 126. In another specific example, the multidomain therapeutic protein can consist essentially of SEQ ID NO: 126. In another specific example, the multidomain therapeutic protein can consist of SEQ ID NO: 126.

Various multidomain therapeutic protein coding sequences are provided. See, e.g., US 2025-0041455, WO 2025/029657, US 2023-0338477, and WO 2023/150623, each of which is herein incorporated by reference in its entirety for all purposes. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 125. In another example, the multidomain therapeutic protein coding sequence is (or comprises a sequence) at least 95%, at least 96%, at least 97%, at least 98%, at least 99%, at least 99.5%, or 100% identical to SEQ ID NO: 125. In another example, the multidomain therapeutic protein coding sequence is (or comprises a sequence) at least 99%, at least 99.5%, or 100% identical to SEQ ID NO: 125. In another example, the multidomain therapeutic protein coding sequence comprises the sequence set forth in SEQ ID NO: 125. In another example, the multidomain therapeutic protein coding sequence consists essentially of the sequence set forth in SEQ ID NO: 125. In another example, the multidomain therapeutic protein coding sequence consists of the sequence set forth in SEQ ID NO: 125. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 126 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 126 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 126 (and, e.g., retaining the activity of native GAA). Optionally, the multidomain therapeutic protein coding sequence in the above examples encodes a multidomain therapeutic protein comprising the sequence set forth in SEQ ID NO: 126. Optionally, the multidomain therapeutic protein coding sequence in the above examples encodes a multidomain therapeutic protein consisting essentially of the sequence set forth in SEQ ID NO: 126. Optionally, the multidomain therapeutic protein coding sequence in the above examples encodes a multidomain therapeutic protein consisting of the sequence set forth in SEQ ID NO: 126. In some embodiments, the nucleotide at position 1857 is a “G.” In some embodiments, the nucleotide at position 1860 is a “C.” In some embodiments, the nucleotide at position 3105 is a “G.” In some embodiments, the nucleotide at position 1857 is a “G,” the nucleotide at position 1860 is a “C,” and the nucleotide at position 3105 is a “G.”

The nucleic acid construct can comprise, for example, (1) a 5′ ITR, (2) a splice acceptor site (e.g., a mouse Alb exon 2 splice acceptor), (3) the multidomain therapeutic protein coding sequence, (4) a polyadenylation signal (e.g., a BGH polyadenylation signal, or a combination of a BGH polyadenylation signal and a unidirectional SV40 late polyadenylation signal), and (5) a 3′ ITR. In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 130 or 133. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 130 or 133 and encodes a multidomain therapeutic protein (or a multidomain therapeutic protein comprising a sequence) at least 99%, at least 99.5%, or 100% identical to SEQ ID NO: 126. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 130 or 133 and encodes a multidomain therapeutic protein comprising the sequence set forth in SEQ ID NO: 126. In another example, the nucleic acid construct comprises a sequence at least 95%, at least 96%, at least 97%, at least 98%, at least 99%, at least 99.5%, or 100% identical to SEQ ID NO: 130 or 133. In another example, the nucleic acid construct comprises a sequence at least 95%, at least 96%, at least 97%, at least 98%, at least 99%, at least 99.5%, or 100% identical to SEQ ID NO: 130 or 133 and encodes a multidomain therapeutic protein (or a multidomain therapeutic protein comprising a sequence) at least 99%, at least 99.5%, or 100% identical to SEQ ID NO: 126. In another example, the nucleic acid construct comprises a sequence at least 95%, at least 96%, at least 97%, at least 98%, at least 99%, at least 99.5%, or 100% identical to SEQ ID NO: 130 or 133 and encodes a multidomain therapeutic protein comprising the sequence set forth in SEQ ID NO: 126. In another example, the nucleic acid construct comprises a sequence at least 99%, at least 99.5%, or 100% identical to SEQ ID NO: 130 or 133. In another example, the nucleic acid construct comprises a sequence at least 99%, at least 99.5%, or 100% identical to SEQ ID NO: 130 or 133 and encodes a multidomain therapeutic protein (or a multidomain therapeutic protein comprising a sequence) at least 99%, at least 99.5%, or 100% identical to SEQ ID NO: 126. In another example, the nucleic acid construct comprises a sequence at least 99%, at least 99.5%, or 100% identical to SEQ ID NO: 130 or 133 and encodes a multidomain therapeutic protein comprising the sequence set forth in SEQ ID NO: 126. In another example, the nucleic acid construct comprises the sequence set forth in SEQ ID NO: 130 or 133. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 126 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 126 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 126 (and, e.g., retaining the activity of native GAA). Optionally, the nucleic acid construct in the above examples encodes a multidomain therapeutic protein comprising the sequence set forth in SEQ ID NO: 126. Optionally, the nucleic acid construct in the above examples encodes a multidomain therapeutic protein consisting essentially of the sequence set forth in SEQ ID NO: 126. Optionally, the nucleic acid construct in the above examples encodes a multidomain therapeutic protein consisting of the sequence set forth in SEQ ID NO: 126.

In a specific example of a multidomain therapeutic protein nucleic acid construct, the encoded multidomain therapeutic protein can comprise SEQ ID NO: 120 or can be at least 90%, at least 91%, at least 92%, at least 93%, at least 94%, at least 95%, at least 96%, at least 97%, at least 98%, at least 99%, or at least 99.5% identical to SEQ ID NO: 120. In another specific example, the multidomain therapeutic protein can consist essentially of SEQ ID NO: 120. In another specific example, the multidomain therapeutic protein can consist of SEQ ID NO: 120.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 128. In another example, the multidomain therapeutic protein coding sequence is (or comprises a sequence) at least 95%, at least 96%, at least 97%, at least 98%, at least 99%, at least 99.5%, or 100% identical to SEQ ID NO: 128. In another example, the multidomain therapeutic protein coding sequence is (or comprises a sequence) at least 99%, at least 99.5%, or 100% identical to SEQ ID NO: 128. In another example, the multidomain therapeutic protein coding sequence comprises the sequence set forth in SEQ ID NO: 128. In another example, the multidomain therapeutic protein coding sequence consists essentially of the sequence set forth in SEQ ID NO: 128. In another example, the multidomain therapeutic protein coding sequence consists of the sequence set forth in SEQ ID NO: 128. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 120 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 120 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 120 (and, e.g., retaining the activity of native GAA). Optionally, the multidomain therapeutic protein coding sequence in the above examples encodes a multidomain therapeutic protein comprising the sequence set forth in SEQ ID NO: 120. Optionally, the multidomain therapeutic protein coding sequence in the above examples encodes a multidomain therapeutic protein consisting essentially of the sequence set forth in SEQ ID NO: 120. Optionally, the multidomain therapeutic protein coding sequence in the above examples encodes a multidomain therapeutic protein consisting of the sequence set forth in SEQ ID NO: 120. In some embodiments, the nucleotide at position 3 is an “A.” In some embodiments, the nucleotide at position 132 is an “A.” In some embodiments, the nucleotide at position 273 is a “T.” In some embodiments, the nucleotide at position 723 is a “G.” In some embodiments, the nucleotide at position 1830 is a “G.” In some embodiments, the nucleotide at position 1833 is a “C.” In some embodiments, the nucleotide at position 3078 is a “G.” In some embodiments, the nucleotide at position 3 is an “A,” the nucleotide at position 132 is an “A,” the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 1830 is a “G,” the nucleotide at position 1833 is a “C,” and the nucleotide at position 3078 is a “G.”

The nucleic acid construct can comprise, for example, (1) a 5′ ITR, (2) a splice acceptor site (e.g., a mouse Alb exon 2 splice acceptor), (3) the multidomain therapeutic protein coding sequence, (4) a polyadenylation signal (e.g., a BGH polyadenylation signal, or a combination of a BGH polyadenylation signal and a unidirectional SV40 late polyadenylation signal), and (5) a 3′ ITR. In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 132 or 134. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 132 or 134 and encodes a multidomain therapeutic protein (or a multidomain therapeutic protein comprising a sequence) at least 99%, at least 99.5%, or 100% identical to SEQ ID NO: 120. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 132 or 134 and encodes a multidomain therapeutic protein comprising the sequence set forth in SEQ ID NO: 120. In another example, the nucleic acid construct comprises a sequence at least 95%, at least 96%, at least 97%, at least 98%, at least 99%, at least 99.5%, or 100% identical to SEQ ID NO: 132 or 134. In another example, the nucleic acid construct comprises a sequence at least 95%, at least 96%, at least 97%, at least 98%, at least 99%, at least 99.5%, or 100% identical to SEQ ID NO: 132 or 134 and encodes a multidomain therapeutic protein (or a multidomain therapeutic protein comprising a sequence) at least 99%, at least 99.5%, or 100% identical to SEQ ID NO: 120. In another example, the nucleic acid construct comprises a sequence at least 95%, at least 96%, at least 97%, at least 98%, at least 99%, at least 99.5%, or 100% identical to SEQ ID NO: 132 or 134 and encodes a multidomain therapeutic protein comprising the sequence set forth in SEQ ID NO: 120. In another example, the nucleic acid construct comprises a sequence at least 99%, at least 99.5%, or 100% identical to SEQ ID NO: 132 or 134. In another example, the nucleic acid construct comprises a sequence at least 99%, at least 99.5%, or 100% identical to SEQ ID NO: 132 or 134 and encodes a multidomain therapeutic protein (or a multidomain therapeutic protein comprising a sequence) at least 99%, at least 99.5%, or 100% identical to SEQ ID NO: 120. In another example, the nucleic acid construct comprises a sequence at least 99%, at least 99.5%, or 100% identical to SEQ ID NO: 132 or 134 and encodes a multidomain therapeutic protein comprising the sequence set forth in SEQ ID NO: 120. In another example, the nucleic acid construct comprises the sequence set forth in SEQ ID NO: 132 or 134. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 120 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 120 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 120 (and, e.g., retaining the activity of native GAA). Optionally, the nucleic acid construct in the above examples encodes a multidomain therapeutic protein comprising the sequence set forth in SEQ ID NO: 120. Optionally, the nucleic acid construct in the above examples encodes a multidomain therapeutic protein consisting essentially of the sequence set forth in SEQ ID NO: 120. Optionally, the nucleic acid construct in the above examples encodes a multidomain therapeutic protein consisting of the sequence set forth in SEQ ID NO: 120.

When specific multidomain therapeutic protein nucleic acid constructs sequences are disclosed herein, they are meant to encompass the sequence disclosed or the reverse complement of the sequence. For example, if a multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (9) Vectors

The nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a polypeptide of interest, a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a polypeptide of interest does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include 10¹², 10¹³, 10¹⁴, 10¹⁵, and 10¹⁶ vector genomes/mL. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg. In another example, the viral titer is about 1E12 to about 2E14 vg/kg (e.g., without CD40 blockade and redosing). In another example, the viral titer is about 3E11 vg/kg to about 5E13 vg/kg (e.g., 2-3× lower with CD40 blockade, due to 2-3 separate administrations with redosing). In another example, the viral titer is about 1E13 vg/kg. In another example, the viral titer is about 3.33E11 to about 5E13 vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette can be derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 110, SEQ ID NO: 111, or SEQ ID NO: 112. Other examples of ITRs comprise one or more mutations compared to SEQ ID NO: 110, SEQ ID NO: 111, or SEQ ID NO: 112 and can be at least 90%, at least 91%, at least 92%, at least 93%, at least 94%, at least 95%, at least 96%, at least 97%, at least 98%, or at least 99% identical to SEQ ID NO: 110, SEQ ID NO: 111, or SEQ ID NO: 112. In some rAAV genomes disclosed herein, the nucleic acid construct is flanked on both sides by the same ITR (i.e., the ITR on the 5′ end, and the reverse complement of the ITR on the 3′ end, such as SEQ ID NO: 110 on the 5′ end and SEQ ID NO: 113 on the 3′ end, or SEQ ID NO: 111 on the 5′ end and SEQ ID NO: 123 on the 3′ end, or SEQ ID NO: 112 on the 5′ end and SEQ ID NO: 124 on the 3′ end). In one example, the ITR on each end can comprise, consist essentially of, or consist of SEQ ID NO: 110 (i.e., SEQ ID NO: 110 on the 5′ end, and the reverse complement on the 3′ end). In another example, the ITR on each end can comprise, consist essentially of, or consist of SEQ ID NO: 111 (i.e., SEQ ID NO: 111 on the 5′ end, and the reverse complement on the 3′ end). In one example, the ITR on at least one end comprises, consists essentially of, or consists of SEQ ID NO: 112. In one example, the ITR on the 5′ end comprises, consists essentially of, or consists of SEQ ID NO: 112. In one example, the ITR on the 3′ end comprises, consists essentially of, or consists of SEQ ID NO: 112. In one example, the ITR on each end can comprise, consist essentially of, or consist of SEQ ID NO: 112 (i.e., SEQ ID NO: 112 on the 5′ end, and the reverse complement on the 3′ end). In other rAAV genomes disclosed herein, the nucleic acid construct is flanked by different ITRs on each end. In one example, the ITR on one end comprises, consists essentially of, or consists of SEQ ID NO: 110, and the ITR on the other end comprises, consists essentially of, or consists of SEQ ID NO: 111. In another example, the ITR on one end comprises, consists essentially of, or consists of SEQ ID NO: 110, and the ITR on the other end comprises, consists essentially of, or consists of SEQ ID NO: 112. In one example, the ITR on one end comprises, consists essentially of, or consists of SEQ ID NO: 111, and the ITR on the other end comprises, consists essentially of, or consists of SEQ ID NO: 112.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8) can be formulated, for example, in 10 mM sodium phosphate, 180 mM sodium chloride, and 0.005% poloxamer 188, at pH 7.3.

### XIV. Nuclease Agents and CRISPR/Cas Systems

The methods and compositions and combinations disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. See, e.g., WO 2023/077012 and US 2023-0149563, each of which is herein incorporated by reference in its entirety for all purposes. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the polypeptide of interest. The polypeptide of interest may be exogenous with respect to its insertion site or locus (target gene), such as a safe harbor locus from which polypeptide of interest is not normally expressed. Alternatively, the polypeptide of interest may be non-exogenous with respect to its insertion site, such as insertion into an endogenous locus encoding the polypeptide of interest to correct a defective gene encoding the polypeptide of interest.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### A. Target Genomic Loci and Albumin (ALB)

Any target genomic locus capable of expressing a gene can be used, such as a safe harbor locus (safe harbor gene, such as ALB) or an endogenous locus that would normally encode the polypeptide interest (e.g., a F9 locus for Factor IX, or a GAA locus for lysosomal alpha-glucosidase). The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67,328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 68).

In another specific example, the target genomic locus is a TTR locus, such as intron 1 of a TTR locus. In a more specific example, the target genomic locus is a human TTR locus, such as intron 1 of a human TTR locus.

### B. Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of different Cas proteins and Cas9 proteins, different modifications to Cas proteins, different forms of providing Cas proteins are provided, e.g., in US 2023-0149563, WO 2023/077012, US 2025-0041455, WO 2025/029657, US 2023-0338477, and WO 2023/150623, each of which is herein incorporated by reference in its entirety for all purposes.

An exemplary Cas protein is a Cas9 protein or a protein derived from a Cas9 protein. Cas9 from S. pyogenes (SpCas9) (e.g., assigned UniProt accession number Q99ZW2) is an exemplary Cas9 protein. An exemplary SpCas9 protein sequence is set forth in SEQ ID NO: 72 (encoded by the DNA sequence set forth in SEQ ID NO: 73). An exemplary SpCas9 mRNA (cDNA) sequence is set forth in SEQ ID NO: 74. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 75. An exemplary SpCas9 mRNA sequence encoding that SpCas9 protein sequence comprises, consists essentially of, or consists of SEQ ID NO: 76. Another exemplary SpCas9 mRNA sequence encoding that SpCas9 protein sequence comprises, consists essentially of, or consists of SEQ ID NO: 65. Another exemplary SpCas9 mRNA sequence encoding that SpCas9 protein sequence comprises SEQ ID NO: 66. An exemplary SpCas9 coding sequence comprises, consists essentially of, or consists of SEQ ID NO: 67.

Other examples of Cas proteins include Cpf1, CasX (Cas12e), and CasΦ (CasPhi or Cas12j).

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 77) or PKKKRRV (SEQ ID NO: 78). The NLS may be a bipartite sequence, such as the NLS of nucleoplasmin, KRPAATKKAGQAKKKK (SEQ ID NO: 79). In a specific example, a single PKKKRKV (SEQ ID NO: 77) NLS may be linked at the C-terminus of the Cas protein. One or more linkers are optionally included at the fusion site.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Cas proteins provided as mRNAs can be modified for improved stability and/or immunogenicity properties. Examples of such modifications are provided, e.g., in US 2023-0149563, WO 2023/077012, US 2025-0041455, WO 2025/029657, US 2023-0338477, and WO 2023/150623, each of which is herein incorporated by reference in its entirety for all purposes. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

### C. Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 80) or GUUUUAGAGCUAUGCUGUUUUG (SEQ ID NO: 81). Any of the DNA-targeting segments disclosed herein can be joined to the 5′ end of SEQ ID NO: 80 or 81 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 82), AAACAGCAUAGCAAGUUAAAAUAAGGCUAGUCCGUUAUCAACUUGAAAAAGUGG CACCGAGUCGGUGCUUUU (SEQ ID NO: 83), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 84).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 96, 94, 95, and 97. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 96, 94, 95, and 97. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 96, 94, 95, and 97. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 96, 94, 95, and 97. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 96, 94, 95, and 97. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 96, 94, 95, and 97. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 96, 94, 95, and 97. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 96, 94, 95, and 97. Other exemplary guide RNAs targeting intron 1 of a human ALB gene are described in US 2023-0149563, WO 2023/077012, US 2025-0041455, WO 2025/029657, US 2023-0338477, and WO 2023/150623, each of which is herein incorporated by reference in its entirety for all purposes.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 96. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 96. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 96. Alternatively, a guide RNA targeting intron 1 of a human ALB gene can comprise a DNA-targeting segment that is at least 90% or at least 95% identical to the sequence (DNA-targeting segment) set forth in SEQ ID NO: 96. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 96. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 96. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 96. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 96.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 94. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 94. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 94. Alternatively, a guide RNA targeting intron 1 of a human ALB gene can comprise a DNA-targeting segment that is at least 90% or at least 95% identical to the sequence (DNA-targeting segment) set forth in SEQ ID NO: 94. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 94. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 94. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 94. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 94.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 95. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 95. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 95. Alternatively, a guide RNA targeting intron 1 of a human ALB gene can comprise a DNA-targeting segment that is at least 90% or at least 95% identical to the sequence (DNA-targeting segment) set forth in SEQ ID NO: 95. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 95. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 95. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 95. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 95.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 97. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 97. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 97. Alternatively, a guide RNA targeting intron 1 of a human ALB gene can comprise a DNA-targeting segment that is at least 90% or at least 95% identical to the sequence (DNA-targeting segment) set forth in SEQ ID NO: 97. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 97. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 97. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 97. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 97.

TABLE 3
Human ALB Intron 1 Guide RNAs.

| Guide | SEQ ID NO (DNA- | SEQ ID NO | SEQ ID NO | SEQ ID NO (Guide RNA |
|---|---|---|---|---|
| RNA | Targeting Segment) | (Unmodified sgRNA) | (Modified sgRNA) | Target Sequence) |
| G009844 | 94 | 98 | 102 | 106 |
| G009857 | 95 | 99 | 103 | 107 |
| G009860 | 96 | 100 | 104 | 108 |
| G009874 | 97 | 101 | 105 | 109 |

TABLE 4
Human ALB Intron 1 Guide Sequences.

| Guide Sequence | SEQ ID NO: |
|---|---|
| GAGCAACCUCACUCUUGUCU | 94 |
| AUUUAUGAGAUCAACAGCAC | 95 |
| UAAAGCAUAGUGCAAUGGAU | 96 |
| UAAUAAAAUUCAAACAUCCU | 97 |

TABLE 5
Human ALB Intron 1 sgRNA Sequences.

| Full Sequence | Full Sequence Modified |
|---|---|
| GAGCAACCUCACUCUUGUCUGUUUUAG | mG*mA*mG*CAACCUCACUCUUGUCUGUUUUAGAmGmCmUmA |
| AGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU (SEQ ID | GmAmGmUmCmGmGmUmGmCmU*mU*mU*mU (SEQ ID NO: 102) |
| NO: 98) | |
| AUUUAUGAGAUCAACAGCACGUUUUAG | mA*mU*mU*UAUGAGAUCAACAGCACGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU (SEQ ID NO: | GmAmGmUmCmGmGmUmGmCmU*mU*mU*mU (SEQ ID NO: 103) |
| 99) | |
| UAAAGCAUAGUGCAAUGGAUGUUUUAG | mU*mA*mA*AGCAUAGUGCAAUGGAUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU (SEQ ID NO: | GmAmGmUmCmGmGmUmGmCmU*mU*mU*mU (SEQ ID NO: 104) |
| 100) | |
| UAAUAAAAUUCAAACAUCCUGUUUUAG | mU*mA*mA*UAAAAUUCAAACAUCCUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU (SEQ ID NO: | GmAmGmUmCmGmGmUmGmCmU*mU*mU*mU (SEQ ID NO: 105) |
| 101) | |

Other exemplary guide RNAs targeting intron 1 of a human ALB gene are described in US 2023-0149563, WO 2023/077012, US 2025-0041455, WO 2025/029657, US 2023-0338477, and WO 2023/150623, each of which is herein incorporated by reference in its entirety for all purposes.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 85); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 86); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 87); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 88); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 89); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 90); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 91); or GUUUUAGAGCUAGAAAUAGCAAGUUAAAAUAAGGCUAGUCCGUUAUCAACUUGG CACCGAGUCGGUGC (version 8; SEQ ID NO: 92). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such guide RNA modifications are provided, e.g., in US 2023-0149563, WO 2023/077012, US 2025-0041455, WO 2025/029657, US 2023-0338477, and WO 2023/150623, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 93), where “N” may be any natural or non-natural nucleotide. For example, the totality of N residues comprise a human ALB intron 1 DNA-targeting segment as described herein (e.g., the sequence set forth in SEQ ID NO: 93, wherein the N residues are replaced with the DNA-targeting segment of any one of SEQ ID NOS: 96, 94, 95, and 97, or the DNA-targeting segment of SEQ ID NO: 96. For example, a modified gRNA can comprise the sequence set forth in any one of SEQ ID NOS: 104, 102, 103, and 105, or the sequence set forth in SEQ ID NO: 104 in Table 5. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example, in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As another example, a guide RNA targeting intron 1 of a human ALB gene can comprise, consist essentially of, or consist of the sequence set forth in any one of SEQ ID NOS: 100, 104, 98, 102, 99, 103, 101, and 105. Alternatively, a guide RNA targeting intron 1 of a human ALB gene can comprise, consist essentially of, or consist of a sequence that is at least 75%, at least 80%, at least 85%, at least 90%, or at least 95% identical to the sequence set forth in any one of SEQ ID NOS: 100, 104, 98, 102, 99, 103, 101, and 105. Alternatively, a guide RNA targeting intron 1 of a human ALB gene can comprise, consist essentially of, or consist of a sequence that is at least 90% or at least 95% identical to the sequence set forth in any one of SEQ ID NOS: 100, 104, 98, 102, 99, 103, 101, and 105. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 100, 104, 98, 102, 99, 103, 101, and 105.

As another example, a guide RNA targeting intron 1 of a human ALB gene can comprise, consist essentially of, or consist of the sequence set forth in SEQ ID NO: 100 or 104. Alternatively, a guide RNA targeting intron 1 of a human ALB gene can comprise, consist essentially of, or consist of a sequence that is at least 75%, at least 80%, at least 85%, at least 90%, or at least 95% identical to the sequence set forth in SEQ ID NO: 100 or 104. Alternatively, a guide RNA targeting intron 1 of a human ALB gene can comprise, consist essentially of, or consist of a sequence that is at least 90% or at least 95% identical to the sequence set forth in SEQ ID NO: 100 or 104. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 100 or 104.

As another example, a guide RNA targeting intron 1 of a human ALB gene can comprise, consist essentially of, or consist of the sequence set forth in SEQ ID NO: 98 or 102. Alternatively, a guide RNA targeting intron 1 of a human ALB gene can comprise, consist essentially of, or consist of a sequence that is at least 75%, at least 80%, at least 85%, at least 90%, or at least 95% identical to the sequence set forth in SEQ ID NO: 98 or 102. Alternatively, a guide RNA targeting intron 1 of a human ALB gene can comprise, consist essentially of, or consist of a sequence that is at least 90% or at least 95% identical to the sequence set forth in SEQ ID NO: 98 or 102. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 98 or 102.

As another example, a guide RNA targeting intron 1 of a human ALB gene can comprise, consist essentially of, or consist of the sequence set forth in SEQ ID NO: 99 or 103. Alternatively, a guide RNA targeting intron 1 of a human ALB gene can comprise, consist essentially of, or consist of a sequence that is at least 75%, at least 80%, at least 85%, at least 90%, or at least 95% identical to the sequence set forth in SEQ ID NO: 99 or 103. Alternatively, a guide RNA targeting intron 1 of a human ALB gene can comprise, consist essentially of, or consist of a sequence that is at least 90% or at least 95% identical to the sequence set forth in SEQ ID NO: 99 or 103. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 99 or 103.

As another example, a guide RNA targeting intron 1 of a human ALB gene can comprise, consist essentially of, or consist of the sequence set forth in SEQ ID NO: 101 or 105. Alternatively, a guide RNA targeting intron 1 of a human ALB gene can comprise, consist essentially of, or consist of a sequence that is at least 75%, at least 80%, at least 85%, at least 90%, or at least 95% identical to the sequence set forth in SEQ ID NO: 101 or 105. Alternatively, a guide RNA targeting intron 1 of a human ALB gene can comprise, consist essentially of, or consist of a sequence that is at least 90% or at least 95% identical to the sequence set forth in SEQ ID NO: 101 or 105. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 101 or 105.

### D. Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 69) or N₂₀NGG (SEQ ID NO: 70). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 71) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 69-71, including the 5′ G or GG and the 3′ GG or NGG. Yet other guide RNA target sequences plus PAMs can have between 14 and 20 nucleotides in length of SEQ ID NOS: 69-71.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As another example, a guide RNA targeting intron 1 of a human ALB gene can target the guide RNA target sequence set forth in any one of SEQ ID NOS: 108, 106, 107, and 109. As another example, a guide RNA targeting intron 1 of a human ALB gene can target at least 17, at least 18, at least 19, or at least 20 contiguous nucleotides of the guide RNA target sequence set forth in any one of SEQ ID NOS: 108, 106, 107, and 109. Other exemplary guide RNAs targeting intron 1 of a human ALB gene are described in US 2023-0149563, WO 2023/077012, US 2025-0041455, WO 2025/029657, US 2023-0338477, and WO 2023/150623, each of which is herein incorporated by reference in its entirety for all purposes.

As another example, a guide RNA targeting intron 1 of a human ALB gene can target the guide RNA target sequence set forth in SEQ ID NO: 108. As another example, a guide RNA targeting intron 1 of a human ALB gene can target at least 17, at least 18, at least 19, or at least 20 contiguous nucleotides of the guide RNA target sequence set forth in SEQ ID NO: 108.

As another example, a guide RNA targeting intron 1 of a human ALB gene can target the guide RNA target sequence set forth in SEQ ID NO: 106. As another example, a guide RNA targeting intron 1 of a human ALB gene can target at least 17, at least 18, at least 19, or at least 20 contiguous nucleotides of the guide RNA target sequence set forth in SEQ ID NO: 106.

As another example, a guide RNA targeting intron 1 of a human ALB gene can target the guide RNA target sequence set forth in SEQ ID NO: 107. As another example, a guide RNA targeting intron 1 of a human ALB gene can target at least 17, at least 18, at least 19, or at least 20 contiguous nucleotides of the guide RNA target sequence set forth in SEQ ID NO: 107.

As another example, a guide RNA targeting intron 1 of a human ALB gene can target the guide RNA target sequence set forth in SEQ ID NO: 109. As another example, a guide RNA targeting intron 1 of a human ALB gene can target at least 17, at least 18, at least 19, or at least 20 contiguous nucleotides of the guide RNA target sequence set forth in SEQ ID NO: 109.

TABLE 6
Human ALB Intron 1 Guide RNA Target Sequences.

| Guide RNA Target Sequence | SEQ ID NO: |
|---|---|
| GAGCAACCTCACTCTTGTCT | 106 |
| ATTTATGAGATCAACAGCAC | 107 |
| TAAAGCATAGTGCAATGGAT | 108 |
| TAATAAAATTCAAACATCCT | 109 |

### E. Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a polypeptide of interest as disclosed herein. For example, the lipid nanoparticles can comprise a nuclease agent (e.g., CRISPR/Cas system), can comprise a nucleic acid construct encoding a polypeptide of interest, or can comprise both a nuclease agent (e.g., a CRISPR/Cas system) and a nucleic acid construct encoding a polypeptide of interest. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct encoding a polypeptide of interest as described elsewhere herein. In certain LNPs, the cargo can include an mRNA encoding a Cas nuclease, such as Cas9, a guide RNA or a nucleic acid encoding a guide RNA, and a nucleic acid construct encoding a polypeptide of interest. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG. Examples of different lipid nanoparticle components and formulations are provided, e.g., in US 2023-0149563, WO 2023/077012, US 2025-0041455, WO 2025/029657, US 2023-0338477, and WO 2023/150623, each of which is herein incorporated by reference in its entirety for all purposes.

In some examples, the LNP formulation that is used is designed to be minimally immunogenic. This can be, for example, to prevent an adjuvant effect on the immune response to AAV. For example, the LNP formulation may include an ionizable lipid that does not elicit a strong innate immune response. Examples of such ionizable lipids include, for example, MC3 or LP01.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes.

Some such lipids suitable for use in the LNPs described herein are biodegradable in vivo.

Neutral lipids function to stabilize and improve processing of the LNPs. Helper lipids include lipids that enhance transfection. The Stealth lipids include lipids that alter the length of time the nanoparticles can exist in vivo. Examples of different cationic lipids, neutral lipids, helper lipids, and steal lipids are provided, e.g., in US 2023-0149563, WO 2023/077012, US 2025-0041455, WO 2025/029657, US 2023-0338477, and WO 2023/150623, each of which is herein incorporated by reference in its entirety for all purposes

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used. In another example, an LNP dose of about 0.3 to about 3 mg/kg can be used.

A specific example of a suitable LNP is one in which the cationic lipid is Lipid A, the neutral lipid is DSPC, the helper lipid is cholesterol, and the stealth lipid is PEG2k-DMG, optionally wherein the lipid nanoparticle comprises four lipids at the following molar ratios: about 50 mol % Lipid A, about 9 mol % DSPC, about 38 mol % cholesterol, and about 3 mol % PEG2k-DMG. Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### F. Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance. Exemplary vectors are described in more detail above in the context of nucleic acid constructs, and such vectors can also be used in the context of providing nuclease agents.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a polypeptide of interest. In certain AAVs, the cargo can include a nucleic acid (e.g., DNA) encoding a Cas nuclease, such as Cas9, a DNA encoding a guide RNA (or multiple guide RNAs), and a nucleic acid construct encoding a polypeptide of interest.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### Xv. Methods of Introducing, Integrating, or Expressing a Nucleic Acid Encoding a Polypeptide of Interest in Cells or Subjects and Therapeutic Methods

The CD40 inhibitors (e.g., CD40 antigen-binding molecules), nucleic acid constructs, nuclease agents and CRISPR/Cas systems, and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of treating an enzyme deficiency in a subject, and methods or preventing or reducing the onset of a sign or symptom an enzyme deficiency in a subject. In some embodiments, the subject does not have preexisting immunity against the nucleic acid construct, the polypeptide of interest encoded by the nucleic acid construct, a nuclease agent or one or more nucleic acids encoding the nuclease agent, or a delivery vehicle for the nucleic acid construct, the nuclease agent, or the one or more nucleic acids encoding the nuclease agent. For example, the delivery vehicle can be a recombinant AAV (e.g., AAV comprising a nucleic acid construct described herein). In one example, the enzyme deficiency is FIX deficiency or the disease is hemophilia B. In another example, the enzyme deficiency is GAA deficiency or the disease is Pompe disease. In another example, the enzyme deficiency is FVIII deficiency or the disease is hemophilia A. In other methods (e.g., where a nucleic acid construct encodes a neutralizing antigen-binding protein as disclosed herein), the methods can be for treating an infectious disease (e.g., bacterial or viral) or treating a cancer.

Likewise, the plasma cell depleting agents or combinations comprising plasma cell depleting agents, nucleic acid constructs, nuclease agents and CRISPR/Cas systems, and compositions disclosed herein can be used in methods of introducing a nucleic acid construct encoding a polypeptide of interest in a cell or population of cells in a subject with preexisting immunity against an immunogen (i.e., an immunogen to be administered to the subject, e.g., an immunogenic delivery vehicle such as, e.g., AAV), methods of inserting or integrating a nucleic acid construct encoding a polypeptide of interest into a target genomic locus in a cell or population of cells in a subject with preexisting immunity against an immunogen (i.e., an immunogen to be administered to the subject, e.g., an immunogenic delivery vehicle such as, e.g., AAV), methods of expressing a polypeptide of interest (e.g., from a target genomic locus) in a cell or population of cells in a subject, methods of treating an enzyme deficiency in a subject with preexisting immunity against an immunogen (i.e., an immunogen to be administered to the subject, e.g., an immunogenic delivery vehicle such as, e.g., AAV), and methods or preventing or reducing the onset of a sign or symptom an enzyme deficiency in a subject with preexisting immunity against an immunogen (i.e., an immunogen to be administered to the subject, e.g., an immunogenic delivery vehicle such as, e.g., AAV). For example, the immunogen can be the nucleic acid construct, the polypeptide of interest, a nuclease agent, one or more nucleic acids encoding the nuclease agent, or a delivery vehicle for the nucleic acid construct, the nuclease agent, or the one or more nucleic acids encoding the nuclease agent. Suitable combinations comprising a plasma cell depleting agent are described in more detail elsewhere herein. Such combinations can be further in combination with a CD40 inhibitor such as the specific scenarios described in more detail below. The plasma cell depleting agents or combinations comprising plasma cell depleting agents can inhibit or prevent an immune response to an immunogen (e.g., nucleic acid construct, nuclease agent or CRISPR/Cas system, e.g., in an immunogenic delivery vehicle) (e.g., an immunogenic delivery vehicle) in a subject with preexisting immunity against the immunogen (i.e., an immunogen to be administered to the subject, e.g., an immunogenic delivery vehicle such as, e.g., AAV) in need thereof or can inhibit or prevent generation of antibodies (e.g., neutralizing antibodies) to an immunogen (e.g., nucleic acid construct, nuclease agent or CRISPR/Cas system, e.g., in an immunogenic delivery vehicle) (e.g., an immunogenic delivery vehicle) in a subject with preexisting immunity against the immunogen (i.e., an immunogen to be administered to the subject, e.g., an immunogenic delivery vehicle such as, e.g., AAV) in need thereof. The term “immune response” refers to a response of a cell of the immune system (e.g., a B-cell, T-cell, macrophage or polymorphonucleocyte) to a stimulus such as an immunogen, e.g., an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). An active immune response can be contrasted with passive immunity, which can be acquired through the transfer of substances such as, e.g., an antibody, a transfer factor, a thymic graft, and/or a cytokine from an actively immunized host to a non-immune host. In some embodiments, the immune response is a humoral (antibody producing) immune response and/or a cell-mediated immune response in a subject (e.g., a human).

Whenever administration of a plasma cell depleting agent, a B cell depleting agent, or an immunoglobulin depleting agent is discussed in this disclosure, it is in the context of being administered to a subject with preexisting immunity against an immunogen (i.e., an immunogen to be administered to the subject, e.g., an immunogenic delivery vehicle such as, e.g., AAV). For example, the subject can have preexisting immunity to a nucleic acid construct, a polypeptide of interest, a nuclease agent, one or more nucleic acids encoding the nuclease agent, or a delivery vehicle for the nucleic acid construct, the nuclease agent, or the one or more nucleic acids encoding the nuclease agent. Likewise, whenever administration of a B cell depleting agent or an immunoglobulin depleting agent is discussed in this disclosure, it is in the context of being administered in combination with a plasma cell depleting agent other than the following scenario regarding immunoglobulin depleting agents in which the immunoglobulin depleting agents may or may not be administered in combination with a plasma cell depleting agent.

In some embodiments, a CD40 inhibitor disclosed herein can be used to prevent an antibody response to an immunoglobulin degrading enzyme that is administered to a subject with preexisting immunity against an immunogen (i.e., an immunogen to be administered to the subject, e.g., an immunogenic delivery vehicle such as, e.g., AAV). Examples of such immunoglobulin degrading enzymes are provided elsewhere herein and include, for example, Imlifidase/IdeS/Fabricator, IdeZ, IdeXork, IceMG, CYR-212, CYR-241, s-1117, HNSA-5487, and IdeE/KJ103. In one example, the immunoglobulin degrading enzyme is Imlifidase/IdeS/Fabricator. In some embodiments, the CD40 inhibitor can be administered before administration of the immunoglobulin degrading enzyme to the subject. In some embodiments, the CD40 inhibitor can be administered after administration of the immunoglobulin degrading enzyme to the subject. In some embodiments, the CD40 inhibitor can be administered before administration of the immunoglobulin degrading enzyme to the subject, and the immunoglobulin degrading enzyme can be administered before the administration of the nucleic acid construct and/or the nuclease agent or the one or more nucleic acids encoding the nuclease agent to the subject. In some embodiments, the immunoglobulin depleting agent can be administered before the CD40 inhibitor, and the CD40 inhibitor can be administered before the administration of the nucleic acid construct and/or the nuclease agent or the one or more nucleic acids encoding the nuclease agent to the subject. In a specific example, the subject has preexisting immunity against AAV (e.g., AAV8). CD40 inhibitor (e.g., anti-CD40 antagonist antibodies) is administered to the subject prior to administration of immunoglobulin degrading enzyme (e.g., IdeS) to prevent an antibody response to the immunoglobulin degrading enzyme. The immunoglobulin degrading enzyme can then cleave circulating IgGs, including anti-AAV IgGs. Additional AAVs can then be administered to the subject.

In some methods, the subject has preexisting immunity against the immunoglobulin degrading enzyme (e.g., the immunoglobulin degrading enzyme may be able to cleave low levels of antibodies against itself). In other methods, the subject does not have preexisting immunity against the immunoglobulin degrading enzyme. In some methods, the CD40 inhibitor can be resistant to cleavage by the immunoglobulin degrading enzyme so that it maintains efficacy in the presence of the immunoglobulin degrading enzyme. For example, an anti-CD40 antibody can be engineered to have one or more mutations within the known cleavage site of the immunoglobulin degrading enzyme (e.g., for IdeS, within the CH2 hinge sequence (CPAPELLGGPSVF (SEQ ID NO: 342)). Alternatively, an anti-CD40 antibody can be engineered to swap the sequence of the known cleavage site of the immunoglobulin degrading enzyme with the homologous sequence from another species that is resistant to the immunoglobulin degrading enzyme (e.g., mouse).

An antibody may be capable of both binding and neutralizing a viral particle or a portion thereof (e.g., neutralizing antibody (nAb)). In some embodiments, the antibody may affect pharmacokinetic properties or alter uptake of AAV into different cell types. In some embodiments, neutralizing (or neutralize or neutralization and the like) in the context of the present disclosure may comprise an effect of immunoglobulins, such as antibodies generated in a host immune response, in reducing the efficacy and/or delivery of a viral particle. As an example, neutralization by an at least one nAb described herein may be realized such that the nAb is directed to the viral particle surface (e.g., capsid protein) which may result in aggregation of viral particles and/or may be realized by inhibition of the fusion of viral and a cellular membrane(s) after attachment of the viral particle to a target cell, by inhibition of endocytosis, and/or by inhibition of production of viral progeny. In various embodiments, an antibody generated in a subject's host immune response can play a neutralizing role thereby causing the delivery effectiveness of the viral particle to be reduced or eliminated. In some embodiments, the induced and/or preexisting host immunity may comprise B and/or T cell immune responses described herein. The blockade and/or suppression of induced and/or preexisting host immunity against viral particles or portions thereof, can improve viral transduction and allow for effective re-administration (i.e., re-dosing) of the viral particles during gene therapy.

In any of the methods, the subject can be from any suitable species, such as eukaryotic or mammalian subjects (e.g., non-human mammalian subject or human subject). A mammal can be, for example, a non-human mammal, a human, a rodent, a rat, a mouse, or a hamster. Other non-human mammals include, for example, non-human primates, e.g., monkeys and apes, e.g., cynomolgus macaques. The term “non-human” excludes humans. Specific examples include, but are not limited to, humans, rodents, mice, rats, and non-human primates. In a specific example, the subject is a human. Likewise, cells can be any suitable type of cell. In a specific example, the cell or cells are a liver cell or liver cells such as a hepatocyte or hepatocytes (e.g., human liver cell(s) or human hepatocyte(s)).

The subjects can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the subjects are not neonatal subjects.

In one example, provided herein are methods of introducing a nucleic acid encoding a polypeptide of interest into a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a CD40 inhibitor (e.g., CD40 antigen-binding molecule). The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. The nucleic acid construct can be administered together with a nuclease agent described herein, or can be administered alone. For example, the nucleic acid construct can be one that expresses the polypeptide of interest without being integrated into target genomic locus (e.g., an episomal vector or an expression vector in which the coding sequence for the polypeptide of interest is operably linked to a promoter). In some methods, the nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agents or combination comprising a plasma cell depleting agent (i.e., when the subject has preexisting immunity against an immunogen (i.e., an immunogen to be administered to the subject, e.g., an immunogenic delivery vehicle such as, e.g., AAV)). The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. The nucleic acid construct can be administered together with a nuclease agent described herein, or can be administered alone. For example, the nucleic acid construct can be one that expresses the polypeptide of interest without being integrated into target genomic locus (e.g., an episomal vector or an expression vector in which the coding sequence for the polypeptide of interest is operably linked to a promoter). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of expressing a polypeptide of interest (e.g., from a target genomic locus) in a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a CD40 inhibitor (e.g., CD40 antigen-binding molecule). The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising a plasma cell depleting agent (i.e., when the subject has preexisting immunity against an immunogen (i.e., an immunogen to be administered to the subject, e.g., an immunogenic delivery vehicle such as, e.g., AAV)). The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of inserting or integrating a nucleic acid construct into a target genomic locus in a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a CD40 inhibitor (e.g., CD40 antigen-binding molecule). The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the nucleic acid construct or composition comprising the nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

Also provided are methods of treating an enzyme deficiency in a subject in need thereof. Such methods can comprise administering any of the nucleic acid constructs described herein (or any of the compositions comprising a nucleic acid construct described herein, including, for example, vectors or lipid nanoparticles) to the subject in combination with a CD40 inhibitor (e.g., CD40 antigen-binding molecule) such that a therapeutically effective level of polypeptide of interest expression or a therapeutically effective level of circulating polypeptide of interest is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of polypeptide of interest expression or a therapeutically effective level of circulating polypeptide of interest is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease.

Also provided are methods of preventing or reducing the onset of a sign or symptom of an enzyme deficiency in a subject (e.g., as compared to an untreated, control subject). By preventing is meant the sign or symptom of the enzyme deficiency never becomes present. Such methods can comprise administering any of the nucleic acid constructs described herein (or any of the compositions comprising a nucleic acid construct described herein, including, for example, vectors or lipid nanoparticles) to the subject in combination with a CD40 inhibitor (e.g., CD40 antigen-binding molecule) such that a therapeutically effective level of polypeptide of interest expression or a therapeutically effective level of circulating polypeptide of interest is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) s administered prior to the nucleic acid construct. In another example, the CD40 inhibitor (e.g., CD40 antigen-binding molecule) is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of polypeptide of interest expression or a therapeutically effective level of circulating polypeptide of interest is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Any of the above methods can further comprise one or more subsequent administration steps. The subsequent administration step can comprise, for example, administering the nucleic acid construct to the subject one or more subsequent times until a desired level of expression and/or activity of the polypeptide of interest is achieved in the subject. In one example, the subsequent administration step can comprise administering to the subject one or more subsequent times: (a) the nucleic acid construct; (b) the nuclease agent or the one or more nucleic acids encoding the nuclease agent; and optionally (c) the CD40 inhibitor (e.g., CD40 antigen-binding molecule). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the CD40 inhibitor (e.g., CD40 antigen-binding molecule). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., 7TR)); and optionally (c) the CD40 inhibitor (e.g., CD40 antigen-binding molecule). In one example, the subsequent administration step can comprise administering to the subject one or more subsequent times: (a) the nucleic acid construct; (b) the nuclease agent or the one or more nucleic acids encoding the nuclease agent; and optionally (c) the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., 7TR)); and optionally (c) the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In one example, the subsequent administration step can comprise administering to the subject one or more subsequent times: (a) the nucleic acid construct; (b) the nuclease agent or the one or more nucleic acids encoding the nuclease agent; and optionally (c) the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). When multiple components are administered, they can be administered simultaneously, or one or more components can be administered sequentially in any order. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some embodiments, the dose of the nucleic acid construct (e.g., an AAV vector comprising the nucleic acid construct) in the subsequent administration is higher than the dose of the nucleic acid construct (e.g., an AAV vector comprising the nucleic acid construct) in the initial administration. This can help, for example, in achieving desired expression levels of the encoded polypeptide of interest. In other embodiments, the dose of the nucleic acid construct in the subsequent administration is the same as the dose of the nucleic acid construct in the initial administration. In other embodiments, the dose of the nucleic acid construct in the subsequent administration is lower than the dose of the nucleic acid construct in the initial administration. In some embodiments, the dose of the nuclease agent or the one or more nucleic acids encoding the nuclease agent (e.g., an LNP comprising the nuclease agent or the one or more nucleic acids encoding the nuclease agent) in the subsequent administration is higher than the dose of the nuclease agent or the one or more nucleic acids encoding the nuclease agent (e.g., an LNP comprising the nuclease agent or the one or more nucleic acids encoding the nuclease agent) in the initial administration. In other embodiments, the dose of the nuclease agent or the one or more nucleic acids encoding the nuclease agent in the subsequent administration is the same as the dose of the nuclease agent or the one or more nucleic acids encoding the nuclease agent in the initial administration. In other embodiments, the dose of the nuclease agent or the one or more nucleic acids encoding the nuclease agent in the subsequent administration is lower than the dose of the nucleic acid construct in the initial administration. The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 pg/mL, at least about 1 μg/mL, at least about 1.5 pg/mL, at least about 2 μg/mL, at least about 2.5 pg/mL, at least about 3 μg/mL, at least about 3.5 pg/mL, at least about 4 μg/mL, at least about 4.5 pg/mL, or at least about 5 μg/mL or about 1 to about 5, about 2 to about 5, or about 3 to about 5 μg/mL (e.g., a serum level of at least about 3 μg/mL or about 3 to about 5 μg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 pg/mL, at least about 1 μg/mL, at least about 1.5 pg/mL, at least about 2 μg/mL, at least about 2.5 pg/mL, at least about 3 μg/mL, at least about 3.5 pg/mL, at least about 4 μg/mL, at least about 4.5 pg/mL, or at least about 5 μg/mL (e.g., a serum level of at least about 2 μg/mL or at least about 5 μg/mL or about 2 to about 50 μg/mL).

Alternatively, the subsequent administration step can comprise, for example, administering a second nucleic acid construct comprising a second coding sequence for the polypeptide of interest (e.g., wherein the second coding sequence is different from the first coding sequence) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor (e.g., CD40 antigen-binding molecule). In one example, the subsequent administration step can comprise administering to the subject one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In one example, the subsequent administration step can comprise administering to the subject one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). When multiple components are administered, they can be administered simultaneously, or one or more components can be administered sequentially in any order. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some embodiments, the dose of the second nucleic acid construct (e.g., an AAV vector comprising the nucleic acid construct) in the subsequent administration is higher than the dose of the nucleic acid construct (e.g., an AAV vector comprising the nucleic acid construct) in the initial administration. This can help, for example, in achieving desired expression levels of the polypeptide of interest encoded by the second nucleic acid construct. In other embodiments, the dose of the second nucleic acid construct in the subsequent administration is the same as the dose of the nucleic acid construct in the initial administration. In other embodiments, the dose of the second nucleic acid construct in the subsequent administration is lower than the dose of the nucleic acid construct in the initial administration. In some embodiments, the dose of the nuclease agent or the one or more nucleic acids encoding the nuclease agent (e.g., an LNP comprising the nuclease agent or the one or more nucleic acids encoding the nuclease agent) in the subsequent administration is higher than the dose of the nuclease agent or the one or more nucleic acids encoding the nuclease agent (e.g., an LNP comprising the nuclease agent or the one or more nucleic acids encoding the nuclease agent) in the initial administration. In other embodiments, the dose of the nuclease agent or the one or more nucleic acids encoding the nuclease agent in the subsequent administration is the same as the dose of the nuclease agent or the one or more nucleic acids encoding the nuclease agent in the initial administration. In other embodiments, the dose of the nuclease agent or the one or more nucleic acids encoding the nuclease agent in the subsequent administration is lower than the dose of the nuclease agent or the one or more nucleic acids encoding the nuclease agent in the initial administration. The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 pg/mL, at least about 1 μg/mL, at least about 1.5 pg/mL, at least about 2 μg/mL, at least about 2.5 pg/mL, at least about 3 μg/mL, at least about 3.5 pg/mL, at least about 4 μg/mL, at least about 4.5 pg/mL, or at least about 5 μg/mL or about 1 to about 5, about 2 to about 5, or about 3 to about 5 μg/mL (e.g., a serum level of at least about 3 μg/mL or at least about 5 μg/mL or about 3-5 pg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 pg/mL, at least about 1 μg/mL, at least about 1.5 pg/mL, at least about 2 μg/mL, at least about 2.5 pg/mL, at least about 3 μg/mL, at least about 3.5 pg/mL, at least about 4 μg/mL, at least about 4.5 pg/mL, or at least about 5 μg/mL (e.g., a serum level of at least about 2 μg/mL or at least about 5 μg/mL).

Alternatively, the subsequent administration step can comprise, for example, administering a second nucleic acid construct comprising a coding sequence for a second polypeptide of interest (e.g., that is different from the first polypeptide of interest) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., 7TR)); and optionally (c) the CD40 inhibitor (e.g., CD40 antigen-binding molecule). In one example, the subsequent administration step can comprise administering to the subject one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). In one example, the subsequent administration step can comprise administering to the subject one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)). When multiple components are administered, they can be administered simultaneously, or one or more components can be administered sequentially in any order. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 108 (e.g., G009860). For example, the first nuclease agent can target SEQ ID NO: 108 (e.g., G009860), and the second nuclease agent can target SEQ ID NO: 106 (e.g., G009844) (or vice versa). For example, the first nuclease agent can target SEQ ID NO: 108 (e.g., G009860), and the second nuclease agent can target SEQ ID NO: 107 (e.g., G009857) (or vice versa). For example, the first nuclease agent can target SEQ ID NO: 108 (e.g., G009860), and the second nuclease agent can target SEQ ID NO: 109 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). In some embodiments, the dose of the second nucleic acid construct (e.g., an AAV vector comprising the nucleic acid construct) in the subsequent administration is higher than the dose of the nucleic acid construct (e.g., an AAV vector comprising the nucleic acid construct) in the initial administration. This can help, for example, in achieving desired expression levels of the polypeptide of interest encoded by the second nucleic acid construct. In other embodiments, the dose of the second nucleic acid construct in the subsequent administration is the same as the dose of the nucleic acid construct in the initial administration. In other embodiments, the dose of the second nucleic acid construct in the subsequent administration is lower than the dose of the nucleic acid construct in the initial administration. In some embodiments, the dose of the nuclease agent or the one or more nucleic acids encoding the nuclease agent (e.g., an LNP comprising the nuclease agent or the one or more nucleic acids encoding the nuclease agent) in the subsequent administration is higher than the dose of the nuclease agent or the one or more nucleic acids encoding the nuclease agent (e.g., an LNP comprising the nuclease agent or the one or more nucleic acids encoding the nuclease agent) in the initial administration. In other embodiments, the dose of the nuclease agent or the one or more nucleic acids encoding the nuclease agent in the subsequent administration is the same as the dose of the nuclease agent or the one or more nucleic acids encoding the nuclease agent in the initial administration. In other embodiments, the dose of the nuclease agent or the one or more nucleic acids encoding the nuclease agent in the subsequent administration is lower than the dose of the nuclease agent or the one or more nucleic acids encoding the nuclease agent in the initial administration. The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some methods, the one or more subsequent administration steps is one subsequent administration step. In some methods, the one or more subsequent administration steps is two subsequent administration steps or comprises at least two subsequent administration steps. In some methods, the one or more subsequent administration steps is three subsequent administration steps or comprises at least three subsequent administration steps. In some methods, the one or more subsequent administration steps is four subsequent administration steps or comprises at least four subsequent administration steps.

In some methods, the CD40 inhibitor (e.g., CD40 antigen-binding molecule) is administered in the one or more subsequent administration steps if there is no preexisting CD40 inhibitor (e.g., CD40 antigen-binding molecule) in the subject. In some methods, the CD40 inhibitor (e.g., CD40 antigen-binding molecule) is administered in the one or more subsequent administration steps if preexisting CD40 inhibitor (e.g., CD40 antigen-binding molecule) expression and/or activity levels are below a desired threshold level (i.e., the level necessary to achieve the desired effect). In some methods, the method comprises measuring CD40 inhibitor (e.g., CD40 antigen-binding molecule) expression and/or activity levels prior to the one or more subsequent administration steps. In some methods, the plasma cell depleting agent or combination comprising the plasma cell depleting agent is administered in the one or more subsequent administration steps if there is no plasma cell depleting agent or combination comprising the plasma cell depleting agent in a subject with preexisting immunity against an immunogen (i.e., an immunogen to be administered to the subject, e.g., an immunogenic delivery vehicle such as, e.g., AAV). In some methods, the plasma cell depleting agent or combination comprising the plasma cell depleting agent is administered in the one or more subsequent administration steps if preexisting expression and/or activity levels of plasma cell depleting agent or combination comprising the plasma cell depleting agent are below a desired threshold level (i.e., the level necessary to achieve the desired effect) in a subject with preexisting immunity against an immunogen (i.e., an immunogen to be administered to the subject, e.g., an immunogenic delivery vehicle such as, e.g., AAV). In some methods, the method comprises measuring expression and/or activity levels of the plasma cell depleting agent or combination comprising the plasma cell depleting agent prior to the one or more subsequent administration steps in a subject with preexisting immunity against an immunogen (i.e., an immunogen to be administered to the subject, e.g., an immunogenic delivery vehicle such as, e.g., AAV).

In some methods, a therapeutically effective amount of the nucleic acid construct or the composition comprising the nucleic acid construct or the combination of the nucleic acid construct and the CD40 inhibitor (e.g., CD40 antigen-binding molecule) and the nuclease agent (e.g., CRISPR/Cas system) is administered to the subject. In some methods, a therapeutically effective amount of the nucleic acid construct or the composition comprising the nucleic acid construct or the combination of the nucleic acid construct and the plasma cell depleting agent or combination comprising the plasma cell depleting agent and the nuclease agent (e.g., CRISPR/Cas system) is administered to the subject (i.e., when the subject has preexisting immunity against an immunogen (i.e., an immunogen to be administered to the subject, e.g., an immunogenic delivery vehicle such as, e.g., AAV)). In some methods, a therapeutically effective amount of the nucleic acid construct or the composition comprising the nucleic acid construct or the combination of the nucleic acid construct and the plasma cell depleting agent and the CD40 inhibitor or combination comprising the plasma cell depleting agent and the CD40 inhibitor and the nuclease agent (e.g., CRISPR/Cas system) is administered to the subject (i.e., when the subject has preexisting immunity against an immunogen (i.e., an immunogen to be administered to the subject, e.g., an immunogenic delivery vehicle such as, e.g., AAV)). A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. Use multiple administration steps can, in some methods, enable use of lower doses of nucleic acid construct and/or nuclease agent for administration to the subject as compared to methods in which only a single administration step is used. For example, if 2-3 administration steps are used, the dose of nucleic acid construct and/or nuclease agent can, in some methods, be 2-3× lower than the dose used in methods in which only a single administration step is used.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

In any of the methods disclosed herein, the nuclease agent can be administered in any form (e.g., one or more nucleic acids encoding the nuclease agent). In any of the methods disclosed herein, plasma cell depleting agents (e.g., anti-BCMA×CD3) or combinations comprising plasma cell depleting agents (e.g., combinations also comprising B cell depleting agents and/or immunoglobulin depleting agents as disclosed elsewhere herein) can be used in combination with CD40 inhibitors in different manners.

In a first example, a plasma cell depleting agent or combination comprising the plasma cell depleting agent can be used to eliminate preexisting immunity against an immunogen (e.g., AAV, such as an AAV nucleic acid construct), while a CD40 inhibitor can be used to prevent any new antibody response to the immunogen on subsequent immunogen exposure. The immunogen can be, for example, the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent. For example, in a subject with preexisting antibody responses to an immunogen, the plasma cell depleting agent or combination comprising the plasma cell depleting agent can be administered to a subject reduce an anti-immunogen antibody response. Once anti-immunogen antibodies reach sub-neutralizing levels, a CD40 inhibitor can be administered to the subject, and the immunogen (e.g., therapeutic AAV, such as an AAV nucleic acid construct) can be dosed thereafter, and a new antibody response against the immunogen is thereby prevented. The plasma cell depleting agent or combination comprising the plasma cell depleting agent returns the subject with preexisting immunity to an effectively immunologically naive status, while the CD40 inhibitor preserves naive status upon immunogen exposure, therefore allowing for immunogen (e.g., AAV) re-dosing in the future. In some embodiments, the plasma cell depleting agent or combination comprising the plasma cell depleting agent are both administered prior to the nucleic acid construct and/or nuclease agent. In some embodiments, the plasma cell depleting agent or combination comprising the plasma cell depleting agent are both administered prior to the nucleic acid construct. In some embodiments, the plasma cell depleting agent or combination comprising the plasma cell depleting agent are both administered prior to the nuclease agent. In some embodiments, the plasma cell depleting agent or combination comprising the plasma cell depleting agent are both administered prior to the nucleic acid construct and nuclease agent. In some embodiments, the plasma cell depleting agent or combination comprising the plasma cell depleting agent is administered prior to the CD40 inhibitor. In some embodiments, the plasma cell depleting agent or combination comprising the plasma cell depleting agent is administered simultaneously with the CD40 inhibitor.

In a second example, a CD40 inhibitor can be administered together with a plasma cell depleting agent or combination comprising the plasma cell depleting agent to block ongoing antibody responses to an immunogen (e.g., AAV) from recent exposure. The immunogen can be, for example, the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent. For example, in a subject with preexisting antibody responses to an immunogen (e.g., AAV), the plasma cell depleting agent or combination comprising the plasma cell depleting agent can be administered with the CD40 inhibitor in a single or repeated dose regimen. Whereas the plasma cell depleting agent or combination comprising the plasma cell depleting agent depletes antibody-secreting cell populations, the CD40 inhibitor prevents new B cell activation and terminates ongoing B cell responses that would not otherwise be impacted by the plasma cell depleting agent treatment alone. In some embodiments, the plasma cell depleting agent or combination comprising the plasma cell depleting agent are both administered prior to the nucleic acid construct and/or nuclease agent. In some embodiments, the plasma cell depleting agent or combination comprising the plasma cell depleting agent are both administered prior to the nucleic acid construct. In some embodiments, the plasma cell depleting agent or combination comprising the plasma cell depleting agent are both administered prior to the nuclease agent. In some embodiments, the plasma cell depleting agent or combination comprising the plasma cell depleting agent are both administered prior to the nucleic acid construct and nuclease agent. In some embodiments, the plasma cell depleting agent or combination comprising the plasma cell depleting agent is administered prior to the CD40 inhibitor. In some embodiments, the plasma cell depleting agent or combination comprising the plasma cell depleting agent is administered simultaneously with the CD40 inhibitor.

In a third example, a plasma cell depleting agent or combination comprising the plasma cell depleting agent can be used to eliminate potential residual antibody responses generated following immunogen (e.g., AAV, such as an AAV nucleic acid construct) exposure in the presence of CD40 blockade, thereby rescuing the ability to re-dose the immunogen. The immunogen can be, for example, the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent. For example, a CD40 inhibitor can be administered to a subject (e.g., naive subject without preexisting immunity to an immunogen) prior to treatment with an immunogen (e.g., AAV, such as an AAV nucleic acid construct). In some embodiments, after immunogen exposure, it is possible that reduced but measurable antibody titers are generated to the immunogen (e.g., to the AAV capsid). Treatment with the plasma depleting agent or combination comprising the plasma cell depleting agent can then be initiated to bring residual antibody titers to sub-neutralizing levels. A second immunogen (either the same or different, such as a second AAV therapeutic that is either the same or different (e.g., a second rAAV8 nucleic acid construct that is different from a first rAAV8 nucleic acid construct)) can then be administered. In such scenarios where CD40 blockade attenuates but does not fully eliminate antibody titers to the immunogen, the plasma cell depleting agent or combination comprising the plasma cell depleting agent can be used to reduce titers to levels that enable immunogen re-dosing. In some embodiments, the CD40 inhibitor is administered prior to the nucleic acid construct (e.g., a first dose of the nucleic acid construct). In some embodiments, the plasma cell depleting agent or combination comprising the plasma cell depleting agent is administered after the first dose of the nucleic acid construct and prior to a second dose of the nucleic acid construct. In some embodiments, the plasma cell depleting agent or combination comprising the plasma cell depleting agent is administered after the first dose of the nucleic acid construct (e.g., a first rAAV8 nucleic acid construct) and prior to administration of a second nucleic acid construct (e.g., a second rAAV8 nucleic acid construct that is different from the first). In some embodiments, the CD40 inhibitor is administered prior to the nuclease agent (e.g., a first dose of the nuclease agent). In some embodiments, the plasma cell depleting agent or combination comprising the plasma cell depleting agent is administered after the first dose of the nuclease agent and prior to a second dose of the nuclease agent. In some embodiments, the plasma cell depleting agent or combination comprising the plasma cell depleting agent is administered after the first dose of the nuclease agent and prior to administration of a second nuclease agent. In some embodiments, the CD40 inhibitor is administered prior to the nucleic acid construct and the nuclease agent (e.g., a first dose of the nucleic acid construct and the nuclease agent). In some embodiments, the plasma cell depleting agent or combination comprising the plasma cell depleting agent is administered after the first dose of the nucleic acid construct and the nuclease agent and prior to a second dose of the nucleic acid construct and the nuclease agent. In some embodiments, the plasma cell depleting agent or combination comprising the plasma cell depleting agent is administered after the first dose of the nucleic acid construct and the nuclease agent and prior to administration of a second dose of the nucleic acid construct and a second nuclease agent. In some embodiments, the plasma cell depleting agent or combination comprising the plasma cell depleting agent is administered after the first dose of the nucleic acid construct and the nuclease agent and prior to administration of a second dose of the nuclease agent and a second nucleic acid construct.

In methods in which a nucleic acid construct is genomically integrated, any target genomic locus capable of expressing a gene can be used, such as a safe harbor locus (safe harbor gene) or an endogenous locus that would normally encode the polypeptide of interest (e.g., a F9 locus for Factor IX). Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the multidomain therapeutic protein in the nucleic acid construct.

Targeted insertion of the nucleic acid construct comprising the coding sequence for the polypeptide of interest into a target genomic locus, and particularly an endogenous ALB locus, offers multiple advantages. Such methods result in stable modification to allow for stable, long-term expression of the coding sequence for the polypeptide of interest. With respect to the ALB locus, such methods are able to utilize the endogenous ALB promoter and regulatory regions to achieve therapeutically effective levels of expression. For example, the coding sequence for the polypeptide of interest in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the coding sequence for the polypeptide of interest in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the multidomain therapeutic protein coding sequence from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the polypeptide of interest encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the coding sequence for the polypeptide of interest will support expression and secretion of the polypeptide of interest. Splicing between endogenous ALB exon 1 and the integrated coding sequence for the polypeptide of interest creates a chimeric mRNA and protein including the endogenous ALB sequence encoded by exon 1 operably linked to the coding sequence for the polypeptide of interest encoded by the integrated nucleic acid construct.

The nucleic acid construct can be inserted into the target genomic locus by any means, including homologous recombination (HR) and non-homologous end joining (NHEJ) as described elsewhere herein. In a specific example, the nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the coding sequence for the polypeptide of interest in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the coding sequence for the polypeptide of interest. In a specific example, the nucleic acid construct is delivered AAV-mediated delivery, and cleavage of the target sites flanking the coding sequence for the polypeptide of interest can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the coding sequence for the polypeptide of interest is inserted into the target genomic locus in the correct orientation but it is reformed if the coding sequence for the polypeptide of interest is inserted into the target genomic locus in the opposite orientation. This can help ensure that the coding sequence for the is inserted in the correct orientation for expression.

In any of the above methods, the CD40 inhibitor (e.g., CD40 antigen-binding molecule) can be administered simultaneously with the nucleic acid construct and/or nuclease agent (e.g., CRISPR/Cas system) or not simultaneously (e.g., sequentially in any combination). For example, in a method comprising administering a composition or combination comprising a CD40 inhibitor (e.g., CD40 antigen-binding molecule), a nucleic acid construct, and a nuclease agent, they can be administered separately (e.g., the CD40 inhibitor (e.g., CD40 antigen-binding molecule) separately from the nucleic acid construct and/or nuclease agent). For example, the CD40 inhibitor (e.g., CD40 antigen-binding molecule) can be administered prior to the nucleic acid construct and/or nuclease agent, subsequent to the nucleic acid construct and/or nuclease agent, prior to and subsequent to the nucleic acid construct and/or nuclease agent, or at the same time as the nucleic acid construct and/or nuclease agent. Any suitable methods of administering CD40 inhibitors (e.g., CD40 antigen-binding molecules), nucleic acid constructs, and nuclease agents to cells can be used (particularly methods of administering to the liver for the nucleic acid constructs and nuclease agents), and examples of such methods are described in more detail elsewhere herein.

In methods in which a composition or combination comprising a CD40 inhibitor (e.g., CD40 antigen-binding molecule), a nucleic acid construct (or vector or LNP), and a nuclease agent is administered (i.e., in methods in which a CD40 inhibitor (e.g., CD40 antigen-binding molecule), a nucleic acid construct (or vector or LNP), and a nuclease agent are both administered), the CD40 inhibitor (e.g., CD40 antigen-binding molecule), the nucleic acid construct, and the nuclease agent can be administered simultaneously. Alternatively, the CD40 inhibitor (e.g., CD40 antigen-binding molecule) and the nucleic acid construct and the nuclease agent can be administered sequentially in any order. For example, the CD40 inhibitor (e.g., CD40 antigen-binding molecule) can be administered prior to and/or after the nucleic acid construct and/or nuclease agent. In one example, the CD40 inhibitor (e.g., CD40 antigen-binding molecule) can be administered prior to and after the nucleic acid construct and/or nuclease agent. In another example, the CD40 inhibitor (e.g., CD40 antigen-binding molecule) can be administered simultaneously with and after the nucleic acid construct and/or nuclease agent.

In one example, the CD40 inhibitor (e.g., CD40 antigen-binding molecule) can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the nucleic acid construct and/or nuclease agent.

In one example, the CD40 inhibitor (e.g., CD40 antigen-binding molecule) is administered about 4 hours, about 8 hours, about 12 hours, about 18 hours, about 1 day, about 2 days, about 3 days, about 4 days, about 5 days, about 6 days, or about 1 week prior to administering the nucleic acid construct and/or nuclease agent. In another example, the CD40 inhibitor (e.g., CD40 antigen-binding molecule) is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nucleic acid construct and/or nuclease agent. In another example, the CD40 inhibitor (e.g., CD40 antigen-binding molecule) is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nucleic acid construct and/or nuclease agent.

In one example, the CD40 inhibitor (e.g., CD40 antigen-binding molecule) is administered about 1 week prior to administering the nucleic acid construct and/or nuclease agent. In another example, the CD40 inhibitor (e.g., CD40 antigen-binding molecule) is administered within about 1 week prior to administering the nucleic acid construct and/or nuclease agent. In another example, the CD40 inhibitor (e.g., CD40 antigen-binding molecule) is administered at least about 1 day, at least about 2 days, at least about 3 days, at least about 4 days, at least about 5 days, at least about 6 days, or at least about 1 week prior to administering the nucleic acid construct and/or nuclease agent. In another example, the CD40 inhibitor (e.g., CD40 antigen-binding molecule) is administered about 1 day, about 2 days, about 3 days, about 4 days, about 5 days, about 6 days, or about 1 week prior to administering the nucleic acid construct and/or nuclease agent. In another example, the CD40 inhibitor (e.g., CD40 antigen-binding molecule) is administered about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, or about 6 days to about 7 days prior to administering the nucleic acid construct and/or nuclease agent.

In one example, the CD40 inhibitor (e.g., CD40 antigen-binding molecule) is administered about 4 hours, about 8 hours, about 12 hours, about 18 hours, about 1 day, about 2 days, about 3 days, about 4 days, about 5 days, about 6 days, or about 1 week prior to and subsequent to administering the nucleic acid construct and/or nuclease agent. In another example, the CD40 inhibitor (e.g., CD40 antigen-binding molecule) is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering the nucleic acid construct and/or nuclease agent. In another example, the CD40 inhibitor (e.g., CD40 antigen-binding molecule) is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the nucleic acid construct and/or nuclease agent.

In one example, the CD40 inhibitor (e.g., CD40 antigen-binding molecule) is administered about 4 hours, about 8 hours, about 12 hours, about 18 hours, about 1 day, about 2 days, about 3 days, about 4 days, about 5 days, about 6 days, or about 1 week after administering the nucleic acid construct and/or nuclease agent. In another example, the CD40 inhibitor (e.g., CD40 antigen-binding molecule) is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nucleic acid construct and/or nuclease agent. In another example, the CD40 inhibitor (e.g., CD40 antigen-binding molecule) is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nucleic acid construct and/or nuclease agent.

In one example, the CD40 inhibitor (e.g., CD40 antigen-binding molecule) is administered within about 1 month, within about 2 months, within about 3 months, within about 4 months, within about 5 months, within about 6 months, or within about 12 months after administering the nucleic acid construct and/or nuclease agent. In another example, the CD40 inhibitor (e.g., CD40 antigen-binding molecule) is administered within about 6 months after administering the nucleic acid construct and/or nuclease agent. In another example, the CD40 inhibitor (e.g., CD40 antigen-binding molecule) is administered about 1 month, about 2 months, about 3 months, about 4 months, about 5 months, or about 6 months after administering the nucleic acid construct and/or nuclease agent. In another example, the CD40 inhibitor (e.g., CD40 antigen-binding molecule) is administered about 1 to about 6 months, about 2 to about 6 months, about 3 to about 6 months, about 4 to about 6 months, about 5 to about 6 months, about 1 to about 5 months, about 1 to about 4 months, about 1 to about 3 months, or about 1 to about 2 months after administering the nucleic acid construct and/or nuclease agent. In another example, the CD40 inhibitor (e.g., CD40 antigen-binding molecule) is administered more than 6 months after administering the nucleic acid construct and/or nuclease agent. In another example, the CD40 inhibitor (e.g., CD40 antigen-binding molecule) is administered about 1 to about 12 months or about 6 months to about 12 months after administering the nucleic acid construct and/or nuclease agent. In some methods, the method can comprise determining whether the nucleic acid construct and/or the nuclease agent are present in the subject (e.g., from a previous administration). The method can then comprise administering the CD40 inhibitor (e.g., CD40 antigen-binding molecule) if the nucleic acid construct and/or the nuclease agent are still present in the subject. For example, if the nucleic acid construct is delivered in a viral vector (e.g., a recombinant AAV vector), the method can comprise determining whether the viral vector is present in the subject. The method can then comprise administering the CD40 inhibitor (e.g., CD40 antigen-binding molecule) if the viral vector is still present (i.e., detectable) in the subject. For example, if the nucleic acid construct is delivered in a viral vector (e.g., a recombinant AAV vector), the method can comprise determining whether viral capsid protein (e.g., AAV capsid protein) is present in the subject. The method can then comprise administering the CD40 inhibitor (e.g., CD40 antigen-binding molecule) if the capsid protein is still present (i.e., detectable) in the subject. For example, if the nuclease agent is delivered in a lipid nanoparticle, the method can comprise determining whether the lipid nanoparticle components (e.g., PEG) are present in the subject. The method can then comprise administering the CD40 inhibitor (e.g., CD40 antigen-binding molecule) if the lipid nanoparticle components are still present (i.e., detectable) in the subject. For example, if the nuclease agent is delivered in a lipid nanoparticle, the method can comprise determining whether certain lipid nanoparticle components are present in the subject. The method can then comprise administering the CD40 inhibitor (e.g., CD40 antigen-binding molecule) if the components still present (i.e., detectable) in the subject.

In any of the above methods, a plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) simultaneously with the nucleic acid construct and/or nuclease agent (e.g., CRISPR/Cas system) or not simultaneously (e.g., sequentially in any combination). For example, in a method comprising administering a composition or combination comprising a plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) and further comprising administering a nucleic acid construct and a nuclease agent, they can be administered separately (e.g., the plasma cell depleting agent or combination comprising the plasma cell depleting agent separately from the nucleic acid construct and/or nuclease agent). For example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) prior to the nucleic acid construct and/or nuclease agent, subsequent to the nucleic acid construct and/or nuclease agent, prior to and subsequent to the nucleic acid construct and/or nuclease agent, or at the same time as the nucleic acid construct and/or nuclease agent. Any suitable methods of administering plasma cell depleting agents or combinations comprising a plasma cell depleting agent, nucleic acid constructs, and nuclease agents to cells can be used (particularly methods of administering to the liver for the nucleic acid constructs and nuclease agents), and examples of such methods are described in more detail elsewhere herein.

In methods in which a composition or combination comprising a plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) and a nucleic acid construct (or vector or LNP) and a nuclease agent are administered (i.e., in methods in which a plasma cell depleting agent or combination comprising the plasma cell depleting agent, a nucleic acid construct (or vector or LNP), and a nuclease agent are administered), the plasma cell depleting agent or combination comprising the plasma cell depleting agent, the nucleic acid construct, and the nuclease agent can be administered simultaneously. Alternatively, the plasma cell depleting agent or combination comprising the plasma cell depleting agent and the nucleic acid construct and the nuclease agent can be administered sequentially in any order. For example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) prior to and/or after the nucleic acid construct and/or nuclease agent. In one example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) prior to and after the nucleic acid construct and/or nuclease agent. In another example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) simultaneously with and after the nucleic acid construct and/or nuclease agent.

In one example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the nucleic acid construct and/or nuclease agent.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nucleic acid construct and/or nuclease agent.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) within about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 1 day, at least about 2 days, at least about 3 days, at least about 4 days, at least about 5 days, at least about 6 days, or at least about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 day, about 2 days, about 3 days, about 4 days, about 5 days, about 6 days, or about 1 week prior to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, or about 6 days to about 7 days prior to administering the nucleic acid construct and/or nuclease agent.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to and subsequent to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the nucleic acid construct and/or nuclease agent.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nucleic acid construct and/or nuclease agent.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) within about 1 month, within about 2 months, within about 3 months, within about 4 months, within about 5 months, within about 6 months, or within about 12 months after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) within about 6 months after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 month, about 2 months, about 3 months, about 4 months, about 5 months, or about 6 months after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 to about 6 months, about 2 to about 6 months, about 3 to about 6 months, about 4 to about 6 months, about 5 to about 6 months, about 1 to about 5 months, about 1 to about 4 months, about 1 to about 3 months, or about 1 to about 2 months after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) more than 6 months after administering the nucleic acid construct and/or nuclease agent. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 to about 12 months or about 6 months to about 12 months after administering the nucleic acid construct and/or nuclease agent. In some methods, the method can comprise determining whether the nucleic acid construct and/or the nuclease agent are present in the subject (e.g., from a previous administration). The method can then comprise administering the plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) if the nucleic acid construct and/or the nuclease agent are still present in the subject. For example, if the nucleic acid construct is delivered in a viral vector (e.g., a recombinant AAV vector), the method can comprise determining whether the viral vector is present in the subject. The method can then comprise administering the plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) if the viral vector is still present (i.e., detectable) in the subject. For example, if the nucleic acid construct is delivered in a viral vector (e.g., a recombinant AAV vector), the method can comprise determining whether viral capsid protein (e.g., AAV capsid protein) is present in the subject. The method can then comprise administering the plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) if the capsid protein is still present (i.e., detectable) in the subject. For example, if the nuclease agent is delivered in a lipid nanoparticle, the method can comprise determining whether the lipid nanoparticle components (e.g., PEG) are present in the subject. The method can then comprise administering the plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) if the lipid nanoparticle components are still present (i.e., detectable) in the subject. For example, if the nuclease agent is delivered in a lipid nanoparticle, the method can comprise determining whether certain lipid nanoparticle components are present in the subject. The method can then comprise administering the plasma cell depleting agent or combination comprising the plasma cell depleting agent (to a subject with preexisting immunity against an immunogen (i.e., an immunogen to be administered to the subject, e.g., an immunogenic delivery vehicle such as, e.g., AAV)) if the components still present (i.e., detectable) in the subject.

In methods in which a composition or combination comprising a plasma cell depleting agent or combination comprising the plasma cell depleting agent in combination with a CD40 inhibitor is administered, the plasma cell depleting agent or combination comprising the plasma cell depleting agent and the CD40 inhibitor can be administered simultaneously (to a subject with preexisting immunity against an immunogen (i.e., an immunogen to be administered to the subject, e.g., an immunogenic delivery vehicle such as, e.g., AAV)). Alternatively, the plasma cell depleting agent or combination comprising the plasma cell depleting agent and the CD40 inhibitor can be administered sequentially in any order. For example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) prior to and/or after the CD40 inhibitor. In one example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) prior to and after the CD40 inhibitor. In another example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) simultaneously with and after the CD40 inhibitor. In another example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) simultaneously with and before the CD40 inhibitor.

In one example, the plasma cell depleting agent or combination comprising the plasma cell depleting agent can be administered (to a subject with preexisting immunity against an immunogen (i.e., an immunogen to be administered to the subject, e.g., an immunogenic delivery vehicle such as, e.g., AAV)) about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the CD40 inhibitor.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the CD40 inhibitor.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 week prior to administering the CD40 inhibitor. In another example, the plasma cell depleting agent or combination comprising the plasma cell depleting agent is administered within about 1 week prior to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 1 day, at least about 2 days, at least about 3 days, at least about 4 days, at least about 5 days, at least about 6 days, or at least about 1 week prior to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 day, about 2 days, about 3 days, about 4 days, about 5 days, about 6 days, or about 1 week prior to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, or about 6 days to about 7 days prior to administering the CD40 inhibitor.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to and subsequent to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the CD40 inhibitor.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the CD40 inhibitor.

In one example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) within about 1 month, within about 2 months, within about 3 months, within about 4 months, within about 5 months, within about 6 months, or within about 12 months after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) within about 6 months after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 month, about 2 months, about 3 months, about 4 months, about 5 months, or about 6 months after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 to about 6 months, about 2 to about 6 months, about 3 to about 6 months, about 4 to about 6 months, about 5 to about 6 months, about 1 to about 5 months, about 1 to about 4 months, about 1 to about 3 months, or about 1 to about 2 months after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) more than 6 months after administering the CD40 inhibitor. In another example, the plasma cell depleting agent or combination comprising the plasma cell depleting agent is administered (to a subject with preexisting immunity against an immunogen (i.e., an immunogen to be administered to the subject, e.g., an immunogenic delivery vehicle such as, e.g., AAV)) about 1 to about 12 months or about 6 months to about 12 months after administering the CD40 inhibitor.

In any of the above methods, the nucleic acid construct can be administered simultaneously with the nuclease agent (e.g., CRISPR/Cas system) or not simultaneously (e.g., sequentially in any combination). For example, in a method comprising administering a composition comprising the nucleic acid construct and a nuclease agent, they can be administered separately. For example, the nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid construct can be inserted in particular types of cells in the subject. The method and vehicle for introducing nucleic acid construct and/or the nuclease agent into the subject can affect which types of cells in the subject are targeted. In some methods, for example, the nucleic acid construct is inserted into a target genomic locus (e.g., an endogenous ALB locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In methods in which a composition comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the nucleic acid construct and the nuclease agent (e.g., CRISPR/Cas system) can be administered using any suitable delivery system and known method. The nuclease agent components and nucleic acid construct (e.g., the guide RNA, Cas protein, and nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the nucleic acid construct via non-homologous end joining (NHEJ)-mediated insertion or homology-directed repair. Optionally, repair with the nucleic acid construct removes or disrupts the guide RNA target sequence(s) so that alleles that have been targeted cannot be re-targeted by the CRISPR/Cas reagents.

As explained in more detail elsewhere herein, the nucleic acid constructs can comprise deoxyribonucleic acid (DNA) or ribonucleic acid (RNA), they can be single-stranded or double-stranded, and they can be in linear or circular form. The nucleic acid constructs can be naked nucleic acids or can be delivered by viruses, such as AAV. In a specific example, the nucleic acid construct can be delivered via AAV and can be capable of insertion into the target genomic locus (e.g., a safe harbor gene, an ALB gene, or intron 1 of an ALB gene) by non-homologous end joining (e.g., the nucleic acid construct can be one that does not comprise a homology arm).

Some nucleic acid constructs are capable of insertion by non-homologous end joining. In some cases, such nucleic acid constructs do not comprise a homology arm. For example, such nucleic acid constructs can be inserted into a blunt end double-strand break following cleavage with a Cas protein. In a specific example, the nucleic acid construct can be delivered via AAV and can be capable of insertion by non-homologous end joining (e.g., the nucleic acid construct can be one that does not comprise a homology arm).

In another example, the nucleic acid construct can be inserted via homology-independent targeted integration. For example, the nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into a subject (e.g., an animal or mammal, such as a human) or cell a nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a multidomain therapeutic protein nucleic acid construct can be introduced prior to the introduction of a Cas protein and a guide RNA, or it can be introduced following introduction of the Cas protein and the guide RNA (e.g., the multidomain therapeutic protein nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of a molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection can include the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million cells as compared with 7 million cells by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids or proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors that can be useful in accomplishing virus-mediated delivery include retroviruses, adenoviruses, vaccinia viruses, poxviruses, and herpes simplex viruses. The viruses can infect dividing cells, non-dividing cells, or both dividing and non-dividing cells. The viruses can integrate into the host genome, or alternatively, do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. AAVs for use in the methods are discussed in more detail elsewhere herein. In another example, the viral titer is about 1E12 to about 2E14 vg/kg (e.g., without CD40 blockade and redosing). In another example, the viral titer is about 3E11 vg/kg to about 5E13 vg/kg (e.g., 2-3× lower with CD40 blockade, due to 2-3 separate administrations with redosing). In another example, the viral titer is about 1E13 vg/kg. Use of the CD40 inhibitors (e.g., CD40 antigen-binding molecules) allows redosing, permitting step-wise dosing with lower doses (e.g., step-wise dosing of 2-3 doses with each dose being 2-3× lower than a dose would be in a one-time administration (e.g., without CD40 blockade). In another example, the viral titer is about 1E12 to about 2E14 vg/kg (e.g., without plasma cell depletion and redosing). In another example, the viral titer is about 3E11 vg/kg to about 5E13 vg/kg (e.g., 2-3× lower with plasma cell depletion, due to 2-3 separate administrations with redosing). Use of the plasma cell depleting agents or combinations comprising plasma cell depleting agents in subjects with preexisting immunity against an immunogen (i.e., an immunogen to be administered to the subject, e.g., an immunogenic delivery vehicle such as, e.g., AAV) allows redosing, permitting step-wise dosing with lower doses (e.g., step-wise dosing of 2-3 doses with each dose being 2-3× lower than a dose would be in a one-time administration (e.g., without plasma cell depletion). In another example, the viral titer is about 1E12 to about 2E14 vg/kg (e.g., without CD40 inhibition and plasma cell depletion and redosing). In another example, the viral titer is about 3E11 vg/kg to about 5E13 vg/kg (e.g., 2-3× lower with CD40 inhibition and plasma cell depletion, due to 2-3 separate administrations with redosing). In another example, the viral titer is about 3.33E11 to about 5E13 vg/kg. Use of CD40 inhibition and the plasma cell depleting agents or combinations comprising plasma cell depleting agents (i.e., in a subject with preexisting immunity against an immunogen (i.e., an immunogen to be administered to the subject, e.g., an immunogenic delivery vehicle such as, e.g., AAV)) allows redosing, permitting step-wise dosing with lower doses (e.g., step-wise dosing of 2-3 doses with each dose being 2-3× lower than a dose would be in a one-time administration (e.g., without CD40 inhibition and plasma cell depletion).

In some embodiments, a subsequent dose of an immunogenic delivery vehicle such as a vector (e.g., a viral vector such as an AAV vector) described herein may be higher than a first dose of an immunogenic delivery vehicle such as a vector (e.g., a viral vector such as an AAV vector) described herein. In some embodiments, relatively low levels of transduction may be observed for an immunogenic delivery vehicle such as a vector (e.g., a viral vector such as an AAV vector) described herein at a lower dose relative to a higher dose. In some embodiments, a higher dose relative to a lower dose of an immunogenic delivery vehicle such as a vector (e.g., a viral vector such as an AAV vector) described herein may be used to achieve higher levels of transduction.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a multidomain therapeutic protein nucleic acid construct. In certain LNPs, the cargo can include an mRNA encoding a Cas nuclease, such as Cas9, a guide RNA or a nucleic acid encoding a guide RNA, and a multidomain therapeutic protein nucleic acid construct. LNPs for use in the methods are described in more detail elsewhere herein.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used. In another example, an LNP dose of about 0.3 to about 3 mg/kg can be used.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or multidomain therapeutic protein nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example, as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The frequency of administration and the number of dosages can depend on a number of factors. The introduction of nucleic acids or proteins into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the nucleic acid construct (or a single administration of the nucleic acid construct and nuclease agent (e.g., Cas protein and guide RNA)) is sufficient to increase expression of polypeptide of interest to a desirable level. In other methods, more than one administration may be beneficial to maximize therapeutic effect.

The methods disclosed herein can increase polypeptide of interest protein levels and/or polypeptide of interest activity levels in a cell or subject (e.g., circulating, serum, or plasma levels in a subject) and can comprise measuring polypeptide of interest protein levels and/or polypeptide of interest activity levels in a cell or subject (e.g., circulating, serum, or plasma levels in a subject). In one example, the methods result in increased expression of the polypeptide of interest in the subject compared to a method comprising administering an episomal expression vector encoding the polypeptide of interest. For example, the methods can result in increased serum levels of the polypeptide of interest in the subject compared to a method comprising administering an episomal expression vector encoding the polypeptide of interest. The methods can also result in increased polypeptide of interest activity in the subject compared to a method comprising administering an episomal expression vector encoding the polypeptide of interest.

In some methods, polypeptide of interest activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, polypeptide of interest activity and/or expression levels in a subject are increased to about or at least about 40%, about or at least about 50%, about or at least about 75%, or at least about 100%, or more, of normal level.

In some methods, circulating polypeptide of interest levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, polypeptide of interest levels are at least about 1 μg/mL or about 1 μg/mL. In some methods, polypeptide of interest levels are at least about 2 μg/mL or about 2 μg/mL. In some methods, polypeptide of interest levels are at least about 5 μg/mL or about 5 μg/mL. In some methods, polypeptide of interest levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in polypeptide of interest levels of about 2 μg/mL to about 30 μg/mL or 2 μg/mL to about 20 μg/mL. For example, the method can result in polypeptide of interest levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of polypeptide of interest over the subject's baseline expression and/or activity (i.e., expression and/or activity prior to administration). In some methods, the method increases expression and/or activity of polypeptide of interest over the subject's baseline expression and/or activity (i.e., expression and/or activity prior to administration. In some methods, polypeptide of interest activity and/or polypeptide of interest expression or serum levels in a subject are increased by about or at least about 10%, about or at least about 25%, about or at least about 50%, about or at least about 75%, or about or at least about 100%, or more, as compared to the subject's polypeptide of interest expression or serum levels and/or activity before administration (i.e., the subject's baseline levels). In certain embodiments, the loss of function is nearly complete such that a relative activity cannot be determined. In certain embodiments, the level of expression is sufficient to treat at least one sign or symptom resulting from the loss of function of the polypeptide of interest.

In some methods, the method increases expression and/or activity of the polypeptide of interest over the cell's baseline expression and/or activity (i.e., expression and/or activity prior to administration). In some methods, the method increases expression and/or activity of polypeptide of interest over the cell's baseline expression and/or activity (i.e., expression and/or activity prior to administration. In some methods, polypeptide of interest activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the polypeptide of interest activity and/or expression levels before administration (i.e., the subject's baseline levels). In certain embodiments, the polypeptide of interest loss of function is nearly complete such that a relative activity cannot be determined. In certain embodiments, the level of expression is sufficient to treat at least one sign or symptom resulting from the loss of function of the polypeptide of interest.

In a specific example, the polypeptide of interest activity levels in a subject are increased to no more than about 300%, no more than about 250%, no more than about 200%, or no more than about 150% of normal polypeptide of interest activity levels.

In a specific example, the polypeptide of interest activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels. In a specific example, the polypeptide of interest activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels.

In some methods, the method results in increased expression of the polypeptide of interest in the subject (e.g., neonatal subject) compared to a method comprising administering an episomal expression vector encoding the polypeptide of interest in a control subject. In some methods, the method results in increased serum levels of the polypeptide of interest in the subject (e.g., neonatal subject) compared to a method comprising administering an episomal expression vector encoding the polypeptide of interest to a control subject.

In some methods, the method increases expression or activity of the polypeptide of interest over the subject's (e.g., neonatal subject's) baseline expression or activity of the polypeptide of interest (i.e., any percent change in expression that is larger than typical error bars). In some methods, the method results in expression of the polypeptide of interest at a detectable level above zero, e.g., at a statistically significant level, a clinically relevant level.

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased polypeptide of interest activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of polypeptide of interest in a human is achieved by at least 7 days, at least 14 days, or at least 28 days, optionally at least 56 days, at least 80 days, or at least 96 days. In additional methods, the method comprises maintaining polypeptide of interest activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the polypeptide of interest can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the polypeptide of interest can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the polypeptide of interest is maintained at a level higher than the expression or activity of the polypeptide of interest prior to treatment (i.e., the subject's baseline). In some methods, expression or activity of the polypeptide of interest is considered sustained if it is maintained at a therapeutically effective level of expression or activity. Relative durations, in other organisms, are understood based, e.g., on life span and developmental stages, are covered within the disclosure above. In some methods, expression or activity of the polypeptide of interest is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the polypeptide of interest is sustained when the neonatal subject becomes an adult. In some methods, the expression of the polypeptide of interest is sustained for the lifetime of the subject or neonatal subject.

In some methods, the expression or activity of the polypeptide of interest is at least 50% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at one year after the administering. In some methods, the expression or activity of the polypeptide of interest is at least 60% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at two years after the administering. In some methods, the expression or activity of the polypeptide of interest is at least 60% of the expression or activity of the polypeptide of interest at a peak level of expression measured for the subject at 2 years after the administering. In some methods, the expression or activity of the polypeptide of interest is at least 60% of the expression or activity of the polypeptide of interest at a peak level of expression measured for the subject at 24 weeks after the administering.

In some methods involving insertion into an ALB locus, the subject's circulating albumin levels or cell's albumin levels are normal. Such methods may comprise maintaining the subject's circulating albumin levels or the cell's albumin levels within ±5%, ±10%, ±15%, ±20%, or ±50% of normal circulating albumin levels or normal albumin levels. In some methods, the subject's or cell's albumin levels are unchanged as compared to the albumin levels of untreated individuals by at least week 4, at least week 8, at least week 12, or at least week 20. In some methods, the subject's or cell's albumin levels transiently drop and then return to normal levels. In particular, the methods may comprise detecting no significant alterations in levels of plasma albumin.

In some methods, the method further comprises determining whether the subject has immunity against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent prior to the administering of any of the above. In one example, the determining can comprise determining the presence of total binding antibodies (e.g., all IgGs) against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent (e.g., determining the presence of total binding antibodies against an AAV comprising a nucleic acid construct). In another example, the determining can comprise determining the presence of neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent (e.g., determining the presence of neutralizing antibodies against an AAV comprising a nucleic acid construct). In another example, the determining can comprise determining the presence of non-neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent (e.g., determining the presence of non-neutralizing antibodies against an AAV comprising a nucleic acid construct). In some embodiments, non-neutralizing antibodies may impact the transduction pattern of AAVs and/or the biodistribution/uptake of proteins or transgenes. For example, the determining the presence of neutralizing antibodies can comprise determining whether there is an effective level of neutralizing antibody to prevent the intended outcome of insertion of a nucleic acid construct into a genomic locus or expression of the polypeptide of interest encoded by the nucleic acid construct. In some methods, the method further comprises assessing preexisting anti-polypeptide of interest immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant polypeptide of interest protein. In some methods, the subject has previously been administered recombinant polypeptide of interest protein.

In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase). In some embodiments, the subject does not have preexisting anti-AAV immunity (i.e., is naive or seronegative). In some embodiments, the subject does have preexisting AAV immunity.

In some embodiments, the level of AAV transduction in the target cell and/or tissue is increased or maintained by inhibiting or preventing an immune response to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting or preventing an immune response to the AAV in the subject.

In some embodiments, the level of AAV transduction in the target cell and/or tissue is increased or maintained by inhibiting antibody responses to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting antibody responses to the AAV in the subject.

In some embodiments, the level of expression of the polypeptide of interest is increased or maintained by inhibiting or preventing an immune response to the immunogenic delivery vehicle and/or by inhibiting or preventing an immune response to one or more other delivered components (e.g., nucleic acid construct, nuclease agent or CRISPR/Cas system). As a non-limiting example, the level of expression of the polypeptide of interest may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of expression of the polypeptide of interest may be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of expression of the polypeptide of interest may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the immune response to the immunogenic delivery vehicle and/or the immune response to one or more other delivered components (e.g., nucleic acid construct, nuclease agent or CRISPR/Cas system) may be inhibited by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The immune response to the immunogenic delivery vehicle and/or the immune response to one or more other delivered components may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The immune response to the immunogenic delivery vehicle and/or the immune response to one or more other delivered components may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments of the methods for inhibiting or preventing an immune response to an immunogen described herein, the inhibiting of the immune response can comprise suppression of numbers and/or frequencies of plasma cells and/or B cells when the subject has preexisting immunity against an immunogen (i.e., an immunogen to be administered to the subject, e.g., an immunogenic delivery vehicle such as, e.g., AAV).

In some embodiments, the number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the total number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The total number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The total number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, inhibiting the immune response comprises suppression of immunogen-specific IgG and/or IgM responses.

In some embodiments, the responses of IgG and/or IgM may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The responses of IgG may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The responses of IgG may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the CD40 inhibitor described herein can block or suppress CD40 and/or CD40-mediated cellular signaling pathways and/or mechanisms, e.g., cellular signaling pathways and/or mechanisms associated with an immune response. When the antigen-binding molecule blocks CD40 and/or CD40-mediated signaling and/or mechanisms, such blockade can inhibit an immune response (e.g., a humoral and/or cell-mediated immune response) in a subject. The inhibition of an immune response may comprise, for example, disruption of B cell germinal centers and/or prevention of generation of immunogen-specific germinal center B cells. In such instances, without limitation, the number and/or frequency of immunogen-specific germinal center B cells and/or the total number and/or frequency of germinal center B cells may be reduced as compared to when the antigen-binding molecule is absent. In some embodiments, the CD40 inhibitor described herein can reduce alanine aminotransferase activity (ALT) in serum. In some embodiments, the CD40 inhibitor described herein can reduce liver injury associated with anti-transgene T cell responses.

In some embodiments of methods for inhibiting an immune response to an immunogen described herein, the inhibiting of the immune response can comprise suppression of numbers and/or frequencies of an immunogen-specific B cell, e.g., an immunogen-specific germinal center B cell. In some embodiments of methods for inhibiting an immune response to an immunogen described herein, the inhibiting of the immune response can comprise suppression of the magnitude and duration of an anti-AAV8 antibody response.

In some embodiments, the number and/or frequency of immunogen-specific germinal center B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The number and/or frequency of immunogen-specific germinal center B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The number and/or frequency of immunogen-specific germinal center B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the total number and/or frequency of germinal center B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The total number and/or frequency of germinal center B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The total number and/or frequency of germinal center B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, immunogen-specific germinal center B cells and/or germinal center B cells are eliminated. In some embodiments, the number and/or frequency of memory B cells, naive B cells and/or plasma cells are not impacted by the CD40 inhibitor, e.g., the number and/or frequency of memory B cells, naive B cells and/or plasma cells is the same in the presence of the CD40 inhibitor as compared to when the CD40 inhibitor is absent.

In some embodiments, inhibition of an immune response may comprise suppression of a T cell response to an immunogen. In such instances, without limitation, the number and/or frequency of immunogen-specific T cells or the total number and/or frequency of T cells may be reduced in the presence of a CD40 inhibitor as compared to when the CD40 inhibitor is absent. Without wishing to be bound by theory, the mechanism of action of inhibition of an immune response comprising suppression of a T cell response to an immunogen may comprise, e.g., inhibition of antigen presenting cell (APC) licensing, including licensing of dendritic cells (DCs), macrophages, and B cells. CD40 can be expressed on antigen presentation cells (APCs), and CD40L-CD40 interactions between CD4+ T cells and APCs can lead to so-called “licensing” of APCs. APC licensing, especially for dendritic cells (DCs), can enhance their antigen presentation functions and further promote T cell responses, including cytotoxic CD8+ T cell responses. Licensing can also render macrophages more effective killers of pathogens. If DCs are not as mature due to CD40 signaling blockade of licensing, they can default to a more tolerogenic priming state, e.g., they may tolerize T cells instead of activating them. The general mechanism of action can, in some ways, parallel other methods of co-stimulation blockade (e.g., CTLA4-Ig/belatacept/abatacept), which also attempt to block co-stimulation signals to promote tolerogenic priming. If an initial response to a transgene or recombinant protein is blocked, then the gradual exposure to the transgene protein (or protein therapeutic) afterwards may be less immunogenic than if the immune system were exposed all at once. The consequences could be that CD40 blockade may be used more transiently in one or a few doses (rather than repeated administrations or continuous exposure) to prevent long-term immunogenicity to transgene or to immunogenic protein therapeutics. Put another way, the distinction may be preventing responses versus programming the immune system to accept a foreign protein as self.

In some embodiments, the number and/or frequency of immunogen-specific T cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The number and/or frequency of immunogen-specific T cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The number and/or frequency of immunogen-specific T cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the total number and/or frequency of T cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The total number and/or frequency of T cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The total number and/or frequency of T cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, T cell proliferation and/or activation is reduced in the presence of a CD40 inhibitor described herein as compared to when the CD40 inhibitor is absent. In some embodiments, T cell proliferation and/or activation may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. T cell proliferation and/or activation may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. T cell proliferation and/or activation may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the T cell can be a CD4+ T cell, for example, a CD4+Ki-67+ T cell. In some embodiments, the T cell can be a T Follicular Helper cell (TFH cell). In some embodiments, the T cell can be a regulatory T cell (Treg).

In some embodiments, inhibition of an immune response may comprise suppression of an immunogen-specific IFNγ response. In some embodiments, an immunogen-specific IFNγ response is reduced in the presence of a CD40 inhibitor as compared to when the CD40 inhibitor is absent. In some embodiments, an immunogen-specific IFNγ response may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. An immunogen-specific IFNγ response may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. An immunogen-specific IFNγ response may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, inhibition of an immune response may comprise suppression of an immunogen-specific IgG and/or IgM response. In some embodiments, an immunogen-specific IgG and/or IgM response is reduced in the presence of a CD40 inhibitor as compared to when the CD40 inhibitor is absent. In some embodiments, an immunogen-specific IgG and/or IgM response may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. An immunogen-specific IgG and/or IgM response may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. An immunogen-specific IgG and/or IgM response may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In one aspect, the methods inhibit generation of neutralizing antibodies (nAbs) to an immunogen in a subject in need thereof by administering to the subject an effective amount of a CD40 inhibitor. As a non-limiting example, the generation of neutralizing antibodies to an immunogen may be inhibited by about 1%, about 2%, about 3%, about 4%, about 5%, about 6%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The generation of neutralizing antibodies to an immunogen be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The generation of neutralizing antibodies to an immunogen may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%, or more.

In another aspect, the methods prevent and/or suppress an immunogen-specific T cell response in a subject in need thereof by administering to the subject an effective amount of a CD40 inhibitor. In another aspect, the methods prevent and/or suppress an immunogen-specific T cell response in a subject in need thereof by administering to the subject an effective amount of a CD40L inhibitor.

As a non-limiting example, the prevention and/or suppression of an immunogen-specific T cell response may be inhibited by about 1%, about 2%, about 3%, about 4%, about 5%, about 6%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The prevention and/or suppression of an immunogen-specific T cell response may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The prevention and/or suppression of an immunogen-specific T cell response may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%, or more.

In some embodiments, the prevention and/or suppression of the immunogen-specific T cell response comprises suppression of numbers and/or frequencies of the immunogen-specific T cells.

In some embodiments, the numbers and/or frequencies of the immunogen-specific T cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The numbers and/or frequencies of the immunogen-specific T cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The numbers and/or frequencies of the immunogen-specific T cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the immunogen-specific T cells comprise follicular T helper cells (TFH) and/or CD4+ T cells.

In some embodiments, the prevention and/or suppression of the immunogen-specific T cell response comprises prevention and/or suppression of injury and/or inflammation of an organ and/or a tissue in the subject in response to the immunogen.

In some embodiments, the immunogen is an immunogenic delivery vehicle and/or a polypeptide or polynucleotide encoded by a transgene contained within the immunogenic delivery vehicle, and the prevention and/or suppression of the immunogen-specific T cell response comprises increasing or maintaining the level of transgene expression in the subject.

In some embodiments, the prevention and/or suppression of the immunogen-specific T cell response comprises prevention and/or suppression of injury and/or inflammation of an organ and/or tissue in the subject in response to transgene expression in the organ and/or a tissue.

In some embodiments, the tissue is muscle. Non-limiting tissues include adipose tissue, blood/bone marrow, bone/cartilage/joint, brain/spinal cord/cns/bbb, breast, colon, esophagus, eye, heart, kidney, liver, lung/bronchus, lymph node, ovary, pancreas, pbmc, peripheral nervous system, placenta, prostate, rectum, skeletal muscle, skin, small intestine, spleen, stomach, testis, and uterus.

In some embodiments, the organ is liver. Non-limiting organs include the central nervous system (CNS), the peripheral nervous system (PNS), lungs, liver, bone, skeletal and cardiac muscle, and the reticuloendothelial system.

In some embodiments, the effectiveness of re-administration of an immunogen may be increased by about 1%, about 2%, about 3%, about 4%, about 5%, about 6%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The effectiveness of re-administration of an immunogen be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The effectiveness of re-administration of an immunogen may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

### A. Hemophilia B

In some methods disclosed herein, the polypeptide of interest is a Factor IX (FIX) protein disclosed herein, and the enzyme deficiency is FIX deficiency or hemophilia B. See, e.g., WO 2023/077012 and US 2023-0149563, each of which is herein incorporated by reference in its entirety for all purposes. In such methods, the nucleic acid constructs and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of reducing glycogen accumulation in a cell or a population of cells or a tissue in a subject, methods of treating hemophilia B disease or FIX deficiency in a subject, and methods or preventing or reducing the onset of a sign or symptom of hemophilia B or FIX deficiency in a subject.

The compositions disclosed herein (e.g., F9 nucleic acid constructs, or F9 nucleic acid constructs in combination with CD40 inhibitors (e.g., CD40 antigen-binding molecules) and the nuclease agents (e.g., CRISPR/Cas systems)) are useful for the treatment of FIX deficiency or hemophilia B and/or ameliorating at least one symptom associated with FIX deficiency or hemophilia B. Likewise, the F9 nucleic acid constructs and the nuclease agents (e.g., CRISPR/Cas systems) disclosed herein in combination with plasma cell depleting agents or combinations comprising plasma cell depleting agents are useful for the treatment of FIX deficiency or hemophilia B and/or ameliorating at least one symptom associated with FIX deficiency or hemophilia B (i.e., in subjects with preexisting immunity against an immunogen (i.e., an immunogen to be administered to the subject, e.g., an immunogenic delivery vehicle such as, e.g., AAV)). Likewise, the F9 nucleic acid constructs and the nuclease agents (e.g., CRISPR/Cas systems) disclosed herein in combination with CD40 inhibitors and plasma cell depleting agents or combinations comprising plasma cell depleting agents are useful for the treatment of FIX deficiency or hemophilia B and/or ameliorating at least one symptom associated with FIX deficiency or hemophilia B (i.e., in subjects with preexisting immunity against an immunogen (i.e., an immunogen to be administered to the subject, e.g., an immunogenic delivery vehicle such as, e.g., AAV)). Likewise, the compositions disclosed herein can be used for the preparation of a pharmaceutical composition or medicament for treating a subject having FIX deficiency or hemophilia B.

Symptoms and causes, diagnosis, and treatments for hemophilia B are described in WO 2023/077012 and US 2023-0149563, each of which is herein incorporated by reference in its entirety for all purposes. The severity of hemophilia and bleeding manifestations correlates with the degree of clotting factor's activity levels (Table 7). See, e.g., Srivastava et al. (2020) Haemophilia 26.6:1-158, herein incorporated by reference in its entirety for all purposes. In high-income markets, it is estimated that 34% of hemophilia B patients have mild hemophilia, around 31% have a moderate hemophilia, and 33% have a severe hemophilia. See, e.g., World Federation of Hemophilia, “Report on the Annual Global Survey 2019” World Federation of Hemophilia (2020), herein incorporated by reference in its entirety for all purposes.

TABLE 7
Severity Classification of Hemophilia B Based on Clotting Factor Activity Level.

| Severity | FIX Activity Levels | Clinical Symptoms |
|---|---|---|
| Mild | 6-49% of normal, | Typically experience bleeding only after serious injury, trauma, or surgery |
| | 0.06-0.40 IU/mL | May not be diagnosed until well into adulthood. |
| | | Spontaneous bleeding rare, but may occur in patients with less than |
| | | ~15-30% of normal FIX activity levels. |
| Moderate | 1-5% of normal, | Bleed infrequently, and experience prolonged bleeding following minor |
| | 0.01-0.05 IU/mL | surgery or injury. |
| | | Spontaneous bleeds may occur, generally <1 times per month. |
| Severe | <1% of normal, | Experience bleeding after injury and may have frequent spontaneous |
| | <0.01 IU/mL | bleeds several times per month, including in their joints and muscles. |

Provided are methods of treating a FIX deficiency in a subject and methods of treating hemophilia B in a subject and methods of preventing or inhibiting spontaneous bleeding in a subject having hemophilia B. The hemophilia B can be any type of hemophilia B (e.g., mild hemophilia B, moderate hemophilia B, or severe hemophilia B). Hemophilia B is described in more detail elsewhere herein. Such methods can comprise administering any of the F9 nucleic acid constructs described herein (or any of the compositions comprising a F9 nucleic acid construct described herein, including, for example, vectors or lipid nanoparticles) to the subject in combination with a CD40 inhibitor (e.g., CD40 antigen-binding molecule) such that a therapeutically effective level of FIX expression is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of FIX expression is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target gene to create a cleavage site, the F9 nucleic acid construct can be inserted into the cleavage site to create a modified target gene, and FIX protein can be expressed from the modified target gene such that a therapeutically effective level of FIX expression is achieved in the subject. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene such that a therapeutically effective level of FIX expression is achieved in the subject.

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of hemophilia B may comprise alleviating symptoms of hemophilia B. In one specific example, a method of preventing or inhibiting spontaneous bleeding in a subject having hemophilia B is provided. Hemophilia B is described in detail above and refers to a disorder caused by a missing or defective F9 gene or FIX polypeptide. The disorder includes conditions that are inherited and/or acquired (e.g., caused by a spontaneous mutation in the gene). The defective F9 gene or FIX polypeptide can result in reduced FIX level in the plasma and/or a reduced coagulation activity of FIX. Hemophilia B includes mild, moderate, and severe hemophilia B. For example, individuals with less than about 1% active factor are classified as having severe hemophilia, those with about 1-5% active factor have moderate hemophilia, and those with mild hemophilia have between about 5-40% of normal levels of active clotting factor. As used herein, “normal” or “healthy” individuals include those having between 50 and 160% of normal pooled plasma level of FIX activity and antigen levels. In one example, normal plasma FIX levels are about 3-5 pg/mL. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. In some embodiments, the level of FIX (e.g., circulating FIX) can be measured by a coagulation and/or an immunologic assay. FIX procoagulant activity can be determined by the ability of the patient's plasma to correct the clotting time of FIX-deficient plasma.

In some methods, a therapeutically effective amount of the F9 nucleic acid construct or the composition comprising the F9 nucleic acid construct or the combination of the F9 nucleic acid construct and the CD40 inhibitor (e.g., CD40 antigen-binding molecule) and the nuclease agent (e.g., CRISPR/Cas system) is administered to the subject. In some methods, a therapeutically effective amount of the F9 nucleic acid construct or the composition comprising the F9 nucleic acid construct or the combination of the F9 nucleic acid construct and the plasma cell depleting agent or combination comprising the plasma cell depleting agent and the nuclease agent (e.g., CRISPR/Cas system) is administered to the subject (i.e., when the subject has preexisting immunity against an immunogen (i.e., an immunogen to be administered to the subject, e.g., an immunogenic delivery vehicle such as, e.g., AAV)). In some methods, a therapeutically effective amount of the F9 nucleic acid construct or the composition comprising the F9 nucleic acid construct or the combination of the F9 nucleic acid construct and the CD40 inhibitor in combination with the plasma cell depleting agent or combination comprising the plasma cell depleting agent and the nuclease agent (e.g., CRISPR/Cas system) is administered to the subject (i.e., when the subject has preexisting immunity against an immunogen (i.e., an immunogen to be administered to the subject, e.g., an immunogenic delivery vehicle such as, e.g., AAV)). A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311.

The compositions disclosed herein may be administered to relieve or prevent or decrease the severity of one or more of the symptoms of FIX deficiency or hemophilia B. Such symptoms are described in more detail elsewhere herein.

The methods disclosed herein can increase FIX protein levels and/or FIX activity levels in a cell or subject (e.g., circulating, serum, or plasma levels in a subject) and can comprise measuring FIX protein levels and/or activity levels in a cell or subject (e.g., circulating, serum, or plasma levels in a subject). In one example, the effectiveness of the treatment in a subject can be assessed by measuring serum or plasma FIX activity, wherein an increase in the subject's plasma level and/or activity of FIX indicates effectiveness of the treatment. In another example, effectiveness of the treatment can be determined by assessing clotting function in an aPTT assay and/or thrombin generation in an TGA-EA assay. In another example, effectiveness of the treatment can be determined by assessing the level or activity of Factor IX (e.g., circulating FIX) through a coagulation and/or an immunologic assay (e.g., a sandwich immunoassay, ELISA, or MSD).

In normal or healthy individuals, FIX activity and antigen levels vary between about 50% and 160% of normal pooled plasma, which is about 3-5 μg/mL, based on its purification from adult human plasma. See, e.g., Amiral et al. (1984) Clin. Chem. 30(9):1512-1516, herein incorporated by reference in its entirety for all purposes. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. Individuals having less than 50% of normal plasma level of FIX activity and/or antigen levels are classified as having hemophilia. In particular, individuals with less than about 1% active FIX are classified as having severe hemophilia, while those with about 1-5% active FIX have moderate hemophilia. Individuals with mild hemophilia have between about 6-49% of normal levels of active clotting factor. In some embodiments, the level of circulating FIX can be measured by a coagulation and/or an immunologic assay using well known methods.

In some methods, plasma levels of FIX or FIX activity levels in a subject having hemophilia are increased to about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, or more, of normal level.

In some methods, circulating FIX protein levels are increased to about or at least about 0.05, about or at least about 0.1, about or at least about 0.2, about or at least about 0.5, about or at least about 1, about or at least about 2, about or at least about 3, or about or at least about 4 μg/mL. FIX protein levels may reach about 150 μg/mL, or more. In some methods, FIX protein levels are increased to at least about 4 μg/mL or about 4 μg/mL. In some methods, FIX protein levels are increased to about 4 μg/mL to about 5 μg/mL, about 4 μg/mL to 6 μg/mL, about 4 μg/mL to 8 μg/mL, about 4 μg/mL to about 10 μg/mL, or more. In some methods, FIX protein levels are increased to about 0.1 pg/mL to about 10 μg/mL, about 1 μg/mL to about 10 μg/mL, about 0.1 pg/mL to about 6 μg/mL, about 1 μg/mL to about 6 μg/mL, about 2 μg/mL to about 5 μg/mL, or about 3 μg/mL to about 5 μg/mL. For example, the compositions and methods disclosed herein are useful for increasing plasma levels of Factor IX in a subject having hemophilia to about 6, about 7, about 8, about 9, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 75, about 80, about 85, about 90, about 95, about 100, about 105, about 110, about 115, about 120, about 125, about 130, about 135, about 140, about 145, about 150 μg/mL, or more.

In some methods, plasma FIX activity and/or levels in a subject (e.g., having hemophilia) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the subject's plasma level and/or activity of FIX before administration.

In some methods, FIX activity and/or protein levels in a cell or population of cells (e.g., liver cells, or hepatocytes) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the FIX activity and/or protein levels before administration (e.g., a normal level).

Some methods comprise expressing a therapeutically effective amount of FIX protein (e.g., achieving a therapeutically effective level of circulating FIX coagulation activity in an individual). Some methods comprise achieving FIX activity or expression levels of at least about 5% to about 50% of normal or at least about 50% to about 150% of normal. Some methods comprise achieving an increase in FIX activity or expression over the patient's baseline FIX activity or expression of at least about 1% to about 50% of normal FIX activity, or at least about 5% to about 50% of normal FIX activity, or at least about 50% to about 150% of normal FIX activity. Some methods comprise achieving FIX activity or expression levels of between about 40% and about 150% of normal (i.e., between 40% and 150% of normal). Some methods comprise achieving FIX activity or expression levels of between about 40% and about 100% of normal (e.g., between 40% and 100% of normal). Some methods comprise achieving FIX activity levels of between about 40% and about 150% of normal (i.e., between 40% and 150% of normal). Some methods comprise achieving FIX activity levels of between about 40% and about 100% of normal (e.g., between 40% and 100% of normal). Some methods comprise achieving FIX expression levels of between about 40% and about 150% of normal (i.e., between 40% and 150% of normal). Some methods comprise achieving FIX expression levels of between about 40% and about 100% of normal (e.g., between 40% and 100% of normal).

In a specific example, the FIX activity levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal FIX activity levels (e.g., between about 50% to about 150% of normal FIX activity levels).

In a specific example, the FIX activity levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal FIX activity levels (e.g., to or about 100% of normal FIX activity levels).

In a specific example, the plasma FIX levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal plasma FIX levels (e.g., between about 50% to about 150% of normal plasma FIX levels).

In a specific example, the plasma FIX levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal plasma FIX levels (e.g., to or about 100% of normal plasma FIX levels).

In a specific example, the plasma FIX levels in a subject are increased to between about 0.25 μg/mL to about 15 μg/mL, about 0.25 μg/mL to about 14 μg/mL, about 0.25 μg/mL to about 13 μg/mL, about 0.25 μg/mL to about 12 μg/mL, about 0.25 μg/mL to about 11 μg/mL, about 0.25 μg/mL to about 10 μg/mL, about 0.25 μg/mL to about 9 μg/mL, about 0.25 μg/mL to about 8 μg/mL, about 0.25 μg/mL to about 7 μg/mL, about 0.25 μg/mL to about 6 μg/mL, about 0.25 μg/mL to about 5 μg/mL, about 0.25 μg/mL to about 4 μg/mL, about 0.25 μg/mL to about 3 μg/mL, about 0.25 μg/mL to about 2 μg/mL, about 0.25 μg/mL to about 1 μg/mL, about 0.75 μg/mL to about 15 μg/mL, about 0.75 μg/mL to about 14 μg/mL, about 0.75 μg/mL to about 13 μg/mL, about 0.75 μg/mL to about 12 μg/mL, about 0.75 μg/mL to about 11 μg/mL, about 0.75 μg/mL to about 10 μg/mL, about 0.75 μg/mL to about 9 μg/mL, about 0.75 μg/mL to about 8 μg/mL, about 0.75 μg/mL to about 7 μg/mL, about 0.75 μg/mL to about 6 μg/mL, about 0.75 μg/mL to about 5 μg/mL, about 0.75 μg/mL to about 4 μg/mL, about 0.75 μg/mL to about 3 μg/mL, about 0.75 μg/mL to about 2 μg/mL, about 0.75 μg/mL to about 1 μg/mL, about 0.5 μg/mL to about 15 μg/mL, about 0.75 μg/mL to about 15 μg/mL, about 1 μg/mL to about 15 μg/mL, about 2 μg/mL to about 15 μg/mL, about 3 μg/mL to about 15 μg/mL, about 4 μg/mL to about 15 μg/mL, about 5 μg/mL to about 15 μg/mL, about 0.5 μg/mL to about 10 μg/mL, about 0.75 μg/mL to about 10 μg/mL, about 1 μg/mL to about 10 μg/mL, about 2 μg/mL to about 10 μg/mL, about 3 μg/mL to about 10 μg/mL, about 4 μg/mL to about 10 μg/mL, about 5 μg/mL to about 10 μg/mL, about 0.5 μg/mL to about 7.5 μg/mL, about 0.75 μg/mL to about 7.5 μg/mL, about 1 μg/mL to about 7.5 μg/mL, about 2 μg/mL to about 7.5 μg/mL, about 2.5 μg/mL to about 7.5 μg/mL, about 3 μg/mL to about 7.5 μg/mL, about 4 μg/mL to about 7.5 μg/mL, about 5 μg/mL to about 7.5 μg/mL, about 2.5 μg/mL to about 15 μg/mL, about 2.5 μg/mL to about 14 μg/mL, about 2.5 μg/mL to about 13 μg/mL, about 2.5 μg/mL to about 12 μg/mL, about 2.5 μg/mL to about 11 μg/mL, about 2.5 μg/mL to about 10 μg/mL, about 2.5 μg/mL to about 9 μg/mL, about 2.5 μg/mL to about 8 μg/mL, about 2.5 μg/mL to about 7 μg/mL, about 2.5 μg/mL to about 6 μg/mL, about 2.5 μg/mL to about 5 μg/mL, about 3 μg/mL to about 15 μg/mL, about 3 μg/mL to about 14 μg/mL, about 3 μg/mL to about 13 μg/mL, about 3 μg/mL to about 12 μg/mL, about 3 μg/mL to about 11 μg/mL, about 3 μg/mL to about 10 μg/mL, about 3 μg/mL to about 9 μg/mL, about 3 μg/mL to about 8 μg/mL, about 3 μg/mL to about 7 μg/mL, about 3 μg/mL to about 6 μg/mL, about 3 μg/mL to about 5 μg/mL (e.g., between about 2.5 μg/mL to about 7.5 μg/mL or between about 3 μg/mL to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the plasma FIX levels in a subject are increased to between about 0.5 μg/mL to about 10 μg/mL, about 1 μg/mL to about 9 μg/mL, about 2 μg/mL to about 8 μg/mL, about 3 μg/mL to about 7 μg/mL, or about 4 μg/mL to about 6 μg/mL (e.g., to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the FIX activity levels in a subject are increased to at least about 15% of normal FIX activity levels. In a specific example, the plasma FIX levels in a subject are increased to at least about 15% of normal plasma FIX levels. In a specific example, the plasma FIX levels in a subject are increased to at least about 0.75 μg/mL. For example, the method can be a method of preventing or inhibiting spontaneous bleeding in a subject having hemophilia B, and the FIX activity levels in a subject are increased to at least about 15% of normal FIX activity levels or the plasma FIX levels in a subject are increased to at least about 15% of normal plasma FIX levels or the plasma FIX levels in a subject are increased to at least about 0.75 μg/mL.

In a specific example, the FIX activity levels in a subject are increased to no more than about 300%, no more than about 250%, no more than about 200%, or no more than about 150% of normal FIX activity levels. In a specific example, the plasma FIX levels in a subject are increased to no more than about 300%, no more than about 250%, no more than about 200%, or no more than about 150% of normal plasma FIX levels.

In a specific example, a subject has severe hemophilia, and the FIX activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has severe hemophilia, and the plasma FIX levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has severe hemophilia, and the FIX activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has severe hemophilia, and the plasma FIX levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has moderate hemophilia, and the FIX activity levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has moderate hemophilia, and the plasma FIX levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has moderate hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has moderate hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has mild hemophilia, and the FIX activity levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has mild hemophilia, and the plasma FIX levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has mild hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has mild hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

Some methods comprise achieving a durable effect, such as an at least 1 month, at least 2 months, at least 6 months, at least 1 year, or at least 2 year effect. Some methods comprise achieving the therapeutic effect in a durable and sustained manner, such as an at least 1 month, at least 2 months, at least 6 months, at least 1 year, or at least 2 year effect. In some methods, the increased circulating FIX activity and/or expression level is stable for at least 1 month, at least 2 months, at least 6 months, at least 1 year, or more. In some methods, a steady-state activity and/or level of FIX protein is achieved by at least 7 days, at least 14 days, or at least 28 days. In additional methods, the method comprises maintaining FIX activity and/or levels after a single dose for at least 1, at least 2, at least 4, or at least 6 months, or at least 1, at least 2, at least 3, at least 4, or at least 5 years. Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased FIX activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of FIX in a human is achieved by at least 7 days, at least 14 days, or at least 28 days, optionally at least 56 days, at least 80 days, or at least 96 days. In additional methods, the method comprises maintaining FIX activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the FIX can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the FIX can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the FIX is maintained at a level higher than the expression or activity of the FIX prior to treatment (i.e., the subject's baseline). In some methods, expression or activity of the FIX is considered sustained if it is maintained at a therapeutically effective level of expression or activity. Relative durations, in other organisms, are understood based, e.g., on life span and developmental stages, are covered within the disclosure above. In some methods, expression or activity of the FIX is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the FIX, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the FIX is sustained when the neonatal subject becomes an adult. In some methods, the expression of the FIX is sustained for the lifetime of the subject or neonatal subject.

In some methods, the expression or activity of the FIX is at least 50% of the expression or activity of the FIX at a peak level of expression measured for the human subject at 24 weeks after the administering. In some methods, the expression or activity of the FIX is at least 50% of the expression or activity of the FIX at a peak level of expression measured for the human subject at one year after the administering. In some methods, the expression or activity of the FIX is at least 60% of the expression or activity of the FIX at a peak level of expression measured for the human subject at 24 weeks after the administering. In some methods, expression or activity of the FIX is at least 50% of the expression or activity of the FIX at a peak level of expression measured for the human subject at two years after the administering. In some methods, the expression or activity of the FIX is at least 60% of the expression or activity of the FIX at a peak level of expression measured for the human subject at 2 years after the administering. In some methods, the expression or activity of the FIX is at least 60% of the expression or activity of the FIX at a peak level of expression measured for the human subject at 24 weeks after the administering.

In some methods, combination therapies are used comprising the any of the compositions for expressing FIX disclosed herein together with an additional therapy suitable for treating hemophilia B or a FIX deficiency. As one example, the methods of described herein can be combined with the use of other hemostatic agents, blood factors, and medications. For example, the subject may be administered a therapeutically effective amount of one or more factors selected from the group consisting of factor XI, factor XII, prekallikrein, high molecular weight kininogen (HMWK), factor V, factor VII, factor VIII, factor X, factor XIII, factor II, factor VIIa, and von Willebrands factor. Additionally, or alternatively, treatment may further comprise administering a procoagulant, such as an activator of the intrinsic coagulation pathway, including factor Xa, factor IXa, factor XIa, factor XIIa, and VIIIa, prekallekrein, and high-molecular weight kininogen; or an activator of the extrinsic coagulation pathway, including tissue factor, factor VIIa, factor Va, and factor Xa.

### B. Pompe Disease

In some methods disclosed herein, the polypeptide of interest is a multidomain therapeutic protein disclosed herein (e.g., a lysosomal alpha-glucosidase linked to a CD63-binding delivery domain or TfR-binding delivery domain), and the enzyme deficiency is GAA deficiency or Pompe disease. See, e.g., US 2025-0041455, WO 2025/029657, US 2023-0338477, and WO 2023/150623, each of which is herein incorporated by reference in its entirety for all purposes. In such methods, the nucleic acid constructs and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of reducing glycogen accumulation in a cell or a population of cells or a tissue in a subject, methods of treating Pompe disease or GAA deficiency in a subject, and methods or preventing or reducing the onset of a sign or symptom of Pompe disease or GAA deficiency in a subject.

The multidomain therapeutic protein compositions disclosed herein (e.g., multidomain therapeutic protein nucleic acid constructs, or multidomain therapeutic protein nucleic acid constructs in combination with CD40 inhibitors (e.g., CD40 antigen-binding molecules) and nuclease agents (e.g., CRISPR/Cas systems)) are useful for the treatment of GAA deficiency or Pompe disease and/or ameliorating at least one symptom associated with GAA deficiency or Pompe disease (e.g., as compared to a control, untreated subject). Likewise, the multidomain therapeutic protein nucleic acid constructs and nuclease agents (e.g., CRISPR/Cas systems)) disclosed herein in combination with plasma cell depleting agents or combinations comprising plasma cell depleting agents are useful for the treatment of GAA deficiency or Pompe disease and/or ameliorating at least one symptom associated with GAA deficiency or Pompe disease (e.g., as compared to a control, untreated subject) (i.e., in subjects with preexisting immunity against an immunogen (i.e., an immunogen to be administered to the subject, e.g., an immunogenic delivery vehicle such as, e.g., AAV)). Likewise, the multidomain therapeutic protein nucleic acid constructs and nuclease agents (e.g., CRISPR/Cas systems)) disclosed herein in combination with CD40 inhibitors and plasma cell depleting agents or combinations comprising plasma cell depleting agents are useful for the treatment of GAA deficiency or Pompe disease and/or ameliorating at least one symptom associated with GAA deficiency or Pompe disease (e.g., as compared to a control, untreated subject) (i.e., in subjects with preexisting immunity against an immunogen (i.e., an immunogen to be administered to the subject, e.g., an immunogenic delivery vehicle such as, e.g., AAV)). The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are also useful for preventing or reducing the onset of a sign or symptom of GAA deficiency or Pompe disease (e.g., as compared to a control, untreated subject). Likewise, the compositions disclosed herein can be used for the preparation of a pharmaceutical composition or medicament for treating a subject having GAA deficiency or Pompe disease.

With respect to GAA deficiency or Pompe disease, the terms “treat,” “treated,” “treating,” and “treatment,” include the administration of the multidomain therapeutic domain nucleic acid constructs disclosed herein (e.g., together with a CD40 inhibitor (e.g., CD40 antigen-binding molecule) and a nuclease agent disclosed herein) to subjects to prevent or delay the onset of the symptoms, complications, or biochemical indicia of GAA deficiency or Pompe disease, alleviating the symptoms or arresting or inhibiting further development of GAA deficiency or Pompe disease. Treatment may be prophylactic (to prevent or delay the onset of GAA deficiency or Pompe disease, or to prevent the manifestation of clinical or subclinical symptoms thereof) or therapeutic suppression or alleviation of symptoms after the manifestation of GAA deficiency or Pompe disease.

GAA deficiency refers expression and/or activity levels of GAA being lower in the subject (e.g., neonatal subject) than normal GAA expression and/or activity levels, such that the normal functions of GAA are not fully carried out in the subject (e.g., resulting in Pompe disease). Pompe disease is also known as acid maltase deficiency, acid maltase deficiency disease, alpha-1,4-glucosidase deficiency, AMD, deficiency of alpha-glucosidase, GAA deficiency, glycogen storage disease type II, glycogenosis type II, GSD II, GSD2, and Pompe's disease.

Symptoms and causes, diagnosis, and treatments for Pompe disease are described in US 2025-0041455, WO 2025/029657, US 2023-0338477, and WO 2023/150623, each of which is herein incorporated by reference in its entirety for all purposes.

The methods described herein can be used to treat a lysosomal alpha-glucosidase (GAA) deficiency in a subject in need thereof (e.g., a subject with Pompe disease). The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the expressed multidomain therapeutic protein is delivered to and internalized by skeletal muscle, liver, or heart tissue in the subject. In some methods, the expressed multidomain therapeutic protein is delivered to and internalized by skeletal muscle or heart tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by skeletal muscle, liver, and heart tissue in the subject. In some methods, the expressed multidomain therapeutic protein is delivered to and internalized by skeletal muscle and heart tissue in the subject. In some methods, the expressed multidomain therapeutic protein is delivered to and internalized by skeletal muscle, liver, heart, or central nervous system tissue in the subject. In some methods, the expressed multidomain therapeutic protein is delivered to and internalized by skeletal muscle, heart, or central nervous system tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by central nervous system tissue in the subject. In some methods, the expressed multidomain therapeutic protein is delivered to and internalized by skeletal muscle, liver, heart, and central nervous system tissue in the subject. In some methods, the expressed multidomain therapeutic protein is delivered to and internalized by skeletal muscle, heart, and central nervous system tissue in the subject. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a CD40 inhibitor (e.g., CD40 antigen-binding molecule) such that a therapeutically effective level of multidomain therapeutic protein or GAA expression or a therapeutically effective level of circulating multidomain therapeutic protein or GAA is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) s administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of reducing glycogen accumulation in a cell or a population of cells or a tissue in a subject in need thereof (e.g., a subject with Pompe disease). Similarly, provided are methods of reducing glycogen accumulation in a cell or a population of cells. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a CD40 inhibitor (e.g., CD40 antigen-binding molecule) such that a therapeutically effective level of multidomain therapeutic protein or GAA expression or a therapeutically effective level of circulating multidomain therapeutic protein or GAA is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of treating Pompe disease in a subject. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a CD40 inhibitor (e.g., CD40 antigen-binding molecule) such that a therapeutically effective level of multidomain therapeutic protein or GAA expression or a therapeutically effective level of circulating multidomain therapeutic protein or GAA is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of Pompe disease may comprise alleviating symptoms of Pompe disease. Pompe disease is described in detail above and refers to a disorder caused by a missing or defective GAA gene or GAA polypeptide. The defective GAA gene or GAA polypeptide can result in reduced GAA expression and/or an activity of GAA.

Also provided are methods of preventing or reducing the onset of a sign or symptom of Pompe disease in a subject (e.g., as compared to an untreated, control subject). By preventing is meant the sign or symptom of the Pompe disease never becomes present. Such signs and symptoms are well-known and are described in more detail elsewhere herein. The Pompe disease can be any type of Pompe disease (e.g., infantile-onset Pompe disease (classic infantile-onset or non-classic infantile-onset) or late-onset Pompe disease). For example, Pompe disease can be infantile-onset Pompe disease (e.g., classical infantile-onset Pompe disease). Pompe disease is described in more detail elsewhere herein. In some methods, the method prevents or reduces glycogen accumulation in skeletal muscle, liver, or heart tissue in the subject. In some methods, the method prevents or reduces glycogen accumulation in skeletal muscle or heart tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. For example, glycogen accumulation can be prevented or reduced in skeletal muscle, liver, and heart tissue in the subject. For example, glycogen accumulation can be prevented or reduced in skeletal muscle and heart tissue in the subject. In some methods, the method prevents or reduces glycogen accumulation in skeletal muscle, liver, heart, or central nervous system tissue in the subject. In some methods, the method prevents or reduces glycogen accumulation in skeletal muscle, heart, or central nervous system tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. In some methods, the method prevents or reduces glycogen accumulation in central nervous system tissue in the subject. For example, glycogen accumulation can be prevented or reduced in skeletal muscle, liver, heart, and central nervous system tissue in the subject. For example, the onset of glycogen accumulation can be prevented or reduced in skeletal muscle, heart, and central nervous system tissue in the subject. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a CD40 inhibitor (e.g., CD40 antigen-binding molecule) such that a therapeutically effective level of multidomain therapeutic protein or GAA expression or a therapeutically effective level of circulating multidomain therapeutic protein or GAA is achieved in the subject. The CD40 inhibitor (e.g., CD40 antigen-binding molecule) can be administered prior to, simultaneously with, or after the nucleic acid construct. In one example, the CD40 inhibitor (e.g., CD40 antigen-binding molecule) is administered prior to the nucleic acid construct. In another example, the CD40 inhibitor (e.g., CD40 antigen-binding molecule) is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The CD40 inhibitor (e.g., CD40 antigen-binding molecule) can be administered prior to, simultaneously with, or after the nuclease agent. In one example, the CD40 inhibitor (e.g., CD40 antigen-binding molecule) is administered prior to the nuclease agent. In another example, the CD40 inhibitor (e.g., CD40 antigen-binding molecule) is administered prior to and after the nuclease agent. Optionally, such methods can further comprise administering a plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. Likewise, the plasma cell depleting agent or combination comprising the plasma cell depleting agent can be administered prior to, simultaneously with, or after the CD40 inhibitor. In some methods, the plasma cell depleting agent or combination comprising the plasma cell depleting agent is administered prior to the CD40 inhibitor. In some methods, the plasma cell depleting agent or combination comprising the plasma cell depleting agent is administered simultaneously with the CD40 inhibitor. In some methods, the plasma cell depleting agent or combination comprising the plasma cell depleting agent is administered after the CD40 inhibitor. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the CD40 inhibitor (e.g., CD40 antigen-binding molecule) and the nuclease agent (e.g., CRISPR/Cas system) is administered to the subject. In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) and the nuclease agent (e.g., CRISPR/Cas system) is administered to the subject. In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the CD40 inhibitor in combination with the plasma cell depleting agent or combination comprising the plasma cell depleting agent (i.e., when the subject has preexisting immunity against an immunogen (i.e., an immunogen to be administered to the subject, e.g., an immunogenic delivery vehicle such as, e.g., AAV)) and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. In a specific example, serum levels of at least about 2 μg/mL or at least about 5 μg/mL of the multidomain therapeutic protein are considered therapeutically effective and correspond to complete correction of glycogen storage in muscles.

The methods disclosed herein can increase multidomain therapeutic protein or GAA protein levels and/or multidomain therapeutic protein or GAA activity levels in a cell or subject (e.g., circulating, serum, or plasma levels in a subject) and can comprise measuring multidomain therapeutic protein or GAA protein levels and/or multidomain therapeutic protein or GAA activity levels in a cell or subject (e.g., circulating, serum, or plasma levels in a subject). In one example, the methods result in increased expression of the multidomain therapeutic protein in the subject compared to a method comprising administering an episomal expression vector encoding the multidomain therapeutic protein. For example, the methods can result in increased serum levels of the multidomain therapeutic protein in the subject compared to a method comprising administering an episomal expression vector encoding the multidomain therapeutic protein. The methods can also result in increased multidomain therapeutic protein activity or GAA activity in the subject compared to a method comprising administering an episomal expression vector encoding the multidomain therapeutic protein. Levels of circulating multidomain therapeutic protein or GAA activity can be measured by using well-known methods.

In some methods, GAA activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, GAA activity and/or expression levels in a subject are increased to about or at least about 40%, about or at least about 50%, about or at least about 75%, or at least about 100%, or more, of normal level. In certain embodiments, the level of expression or activity is measured in a cell or tissue in which a sign or symptom of the GAA loss of function is present. For example, when the loss of function results in muscle dysfunction, the level or activity of the multidomain therapeutic protein or GAA is measured in a muscle cell. It is understood that depending on the exogenous protein, the level of activity of the multidomain therapeutic protein may not compare 1:1 with a native GAA protein based on weight. In such embodiment, the relative activity of the multidomain therapeutic protein and the native GAA can be compared. In certain embodiments, the loss of function is nearly complete such that a relative activity cannot be determined. In certain embodiments, the comparison is made to an appropriate control subject. Selection of an appropriate control subject is within the ability of those of skill in the art. In certain embodiments, the level of expression is sufficient to treat at least one sign or symptom resulting from the loss of function of the GAA. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry.

In some methods, circulating multidomain therapeutic protein levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, multidomain therapeutic protein levels are at least about 1 μg/mL or about 1 μg/mL. In some methods, multidomain therapeutic protein levels are at least about 2 μg/mL or about 2 μg/mL. In some methods, multidomain therapeutic protein levels are at least about 5 μg/mL or about 5 μg/mL. In some methods, multidomain therapeutic protein levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in multidomain therapeutic protein levels of about 2 μg/mL to about 30 μg/mL or 2 μg/mL to about 20 μg/mL. For example, the method can result in multidomain therapeutic protein levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of GAA or the multidomain therapeutic protein over the subject's baseline expression and/or activity (i.e., expression and/or activity prior to administration). In some methods, the method increases expression and/or activity of GAA over the subject's baseline expression and/or activity (i.e., expression and/or activity prior to administration. In some methods, GAA activity and/or GAA expression or serum levels in a subject are increased by about or at least about 10%, about or at least about 25%, about or at least about 50%, about or at least about 75%, or about or at least about 100%, or more, as compared to the subject's GAA expression or serum levels and/or activity (e.g., GAA activity) before administration (i.e., the subject's baseline levels). It is understood that depending on the multidomain therapeutic protein, the level of activity of the multidomain therapeutic protein may not compare 1:1 with a native protein based on weight. In such embodiment, the relative activity of the multidomain therapeutic protein and the native GAA can be compared. In certain embodiments, the loss of function is nearly complete such that a relative activity cannot be determined. In certain embodiments, the level of expression is sufficient to treat at least one sign or symptom resulting from the loss of function of the GAA.

In some methods, the method increases expression and/or activity of the multidomain therapeutic protein over the cell's baseline expression and/or activity (i.e., expression and/or activity prior to administration). In some methods, the method increases expression and/or activity of GAA over the cell's baseline expression and/or activity (i.e., expression and/or activity prior to administration. In some methods, GAA activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the GAA activity and/or expression levels before administration (i.e., the subject's baseline levels). It is understood that depending on the multidomain therapeutic protein, the level of activity of the multidomain therapeutic protein may not compare 1:1 with a native GAA protein based on weight. In such embodiment, the relative activity of the multidomain therapeutic protein and the native GAA protein can be compared. In certain embodiments, the GAA loss of function is nearly complete such that a relative activity cannot be determined. In certain embodiments, the level of expression is sufficient to treat at least one sign or symptom resulting from the loss of function of the GAA.

In a specific example, the GAA activity levels in a subject are increased to no more than about 300%, no more than about 250%, no more than about 200%, or no more than about 150% of normal GAA activity levels.

In a specific example, the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic infantile-onset Pompe disease), and the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, a subject has infantile-onset Pompe disease (e.g., classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic or non-classic infantile-onset Pompe disease), and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels). In a specific example, a subject has infantile-onset Pompe disease (e.g., classic or non-classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has late-onset Pompe disease, and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels).

In a specific example, a subject has infantile-onset Pompe disease (e.g., classic infantile-onset Pompe disease), and the GAA activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels. In a specific example, a subject has infantile-onset Pompe disease (e.g., classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels

In a specific example, a subject has infantile-onset Pompe disease (e.g., classic or non-classic infantile-onset Pompe disease), and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels). In a specific example, a subject has infantile-onset Pompe disease (e.g., classic or non-classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels.

In a specific example, a subject has late-onset Pompe disease, and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels).

In some methods, the method results in increased expression of the multidomain therapeutic protein in the subject (e.g., neonatal subject) compared to a method comprising administering an episomal expression vector encoding the polypeptide of interest in a control subject. In some methods, the method results in increased serum levels of the multidomain therapeutic protein in the subject (e.g., neonatal subject) compared to a method comprising administering an episomal expression vector encoding the polypeptide of interest to a control subject.

In some methods, the method increases expression or activity of the multidomain therapeutic protein or GAA over the subject's (e.g., neonatal subject's) baseline expression or activity of the multidomain therapeutic protein or GAA (i.e., any percent change in expression that is larger than typical error bars). In some methods, the method results in expression of the multidomain therapeutic protein or GAA at a detectable level above zero, e.g., at a statistically significant level, a clinically relevant level.

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased multidomain therapeutic protein or GAA activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of multidomain therapeutic protein or GAA in a human is achieved by at least 7 days, at least 14 days, or at least 28 days, optionally at least 56 days, at least 80 days, or at least 96 days. In additional methods, the method comprises maintaining multidomain therapeutic protein or GAA activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the multidomain therapeutic protein or GAA can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the multidomain therapeutic protein or GAA can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the multidomain therapeutic protein or GAA is maintained at a level higher than the expression or activity of the multidomain therapeutic protein or GAA prior to treatment (i.e., the subject's baseline). In some methods, expression or activity of the multidomain therapeutic protein or GAA is considered sustained if it is maintained at a therapeutically effective level of expression or activity. Relative durations, in other organisms, are understood based, e.g., on life span and developmental stages, are covered within the disclosure above. In some methods, expression or activity of the multidomain therapeutic protein or GAA is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the multidomain therapeutic protein or GAA is sustained when the neonatal subject becomes an adult. In some methods, the expression of the multidomain therapeutic protein or GAA is sustained for the lifetime of the subject or neonatal subject.

In some methods, the expression or activity of the multidomain therapeutic protein is at least 50% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at one year after the administering. In some methods, the expression or activity of the multidomain therapeutic protein is at least 60% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at two years after the administering. In some methods, the expression or activity of the multidomain therapeutic protein is at least 60% of the expression or activity of the multidomain therapeutic protein at a peak level of expression measured for the subject at 2 years after the administering. In some methods, the expression or activity of the multidomain therapeutic protein is at least 60% of the expression or activity of the multidomain therapeutic protein at a peak level of expression measured for the subject at 24 weeks after the administering.

### C. Dosage and Administration Regimens

In some embodiments, an amount of CD40 inhibitor (e.g., CD40 antigen-binding molecule, e.g., a CD40×CD40 bispecific antigen-binding molecule (e.g., bispecific antibody)) that is administered to a subject according to the methods disclosed herein is a therapeutically effective amount. As used herein, the phrase “therapeutically effective amount” means an amount that produces the desired effect for which it is administered.

In some embodiments, the CD40 inhibitor (e.g., CD40 antigen-binding molecule, e.g., anti-CD40×CD40 bispecific antibody) is administered to a subject as a weight-based dose. A “weight-based dose” (e.g., a dose in mg/kg) is a dose of the CD40 inhibitor (e.g., CD40 antigen-binding molecule) that will change depending on the subject's weight.

In other embodiments, the CD40 inhibitor (e.g., CD40 antigen-binding molecule, e.g., anti-CD40×CD40 bispecific antibody) is administered as a fixed dose. A “fixed dose” (e.g., a dose in mg) means that one dose of the CD40 inhibitor (e.g., CD40 antigen-binding molecule) is used for all subjects regardless of any specific subject-related factors, such as weight. In one particular embodiment, a fixed dose of an CD40 inhibitor (e.g., CD40 antigen-binding molecule) is based on a predetermined weight or age.

Typically, a suitable dose of the CD40 inhibitor (e.g., CD40 antigen-binding molecule) can be in the range of about 0.001 to about 200.0 milligram per kilogram body weight of the recipient, generally in the range of about 1 to 50 mg per kilogram body weight. For example, the CD40 inhibitor (e.g., CD40 antigen-binding molecule) can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure. In some embodiments, the CD40 inhibitor (e.g., CD40 antigen-binding molecule) can be administered at a dose of about 10 mg/kg to about 50 mg/kg. In some embodiments, the CD40 inhibitor (e.g., CD40 antigen-binding molecule) can be administered at a dose of about 20 mg/kg to about 50 mg/kg. In some embodiments, the CD40 inhibitor (e.g., CD40 antigen-binding molecule) can be administered at a dose of about 20 mg/kg. In some embodiments, the CD40 antigen-binding molecule and/or the CD40 inhibitor and/or the immunogen is administered at about 20 mg/kg per single dose. In some embodiments, the CD40 antigen-binding molecule and/or the CD40 inhibitor and/or the immunogen is administered at about 50 mg/kg per single dose.

In some embodiments, the CD40 inhibitor (e.g., CD40 antigen-binding molecule) is administered as a fixed dose of between about 5 mg to about 2500 mg. In some embodiments, the CD40 inhibitor (e.g., CD40 antigen-binding molecule) is administered as a fixed dose of about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

In some embodiments, the CD40 inhibitor (e.g., CD40 antigen-binding molecule, e.g., anti-CD40×CD40 bispecific antibody) is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the CD40 antigen-binding molecule and/or the CD40 inhibitor and/or the immunogen is administered to a subject about once every two weeks. In some embodiments, the CD40 antigen-binding molecule and/or the CD40 inhibitor and/or the immunogen is administered to a subject about once a week. In some embodiments, the CD40 antigen-binding molecule and/or the CD40 inhibitor and/or the immunogen is administered at about 20 mg/kg per single dose to a subject about once every two weeks.

In some embodiments, multiple doses of a CD40 inhibitor (e.g., CD40 antigen-binding molecule, e.g., anti-CD40×CD40 bispecific antibody) are administered to a subject over a defined time course. In some embodiments, the methods of the present disclosure comprise sequentially administering multiple doses of the CD40 inhibitor (e.g., CD40 antigen-binding molecule) to a subject. As used herein, “sequentially administering” means that each dose of the CD40 inhibitor (e.g., CD40 antigen-binding molecule) is administered to the subject at a different point in time, e.g., on different days separated by a predetermined interval (e.g., hours, days, weeks, or months). In some embodiments, the methods of the disclosure comprise sequentially administering to the patient a single initial dose of the CD40 inhibitor (e.g., CD40 antigen-binding molecule), followed by one or more secondary doses of the CD40 inhibitor (e.g., CD40 antigen-binding molecule), and optionally followed by one or more tertiary doses of the CD40 inhibitor (e.g., CD40 antigen-binding molecule). In some embodiments, the methods of the present disclosure comprise sequentially administering multiple doses of the CD40 inhibitor (e.g., CD40 antigen-binding molecule) to a subject prior to administering the nucleic acid construct or the nuclease agent or one or more nucleic acids encoding the nuclease agent. In some embodiments, the methods of the present disclosure comprise sequentially administering multiple doses of the CD40 inhibitor (e.g., CD40 antigen-binding molecule) to a subject prior to administering the nucleic acid construct (e.g., prior to AAV exposure). In some embodiments, the methods of the present disclosure comprise sequentially administering multiple doses of the CD40 inhibitor (e.g., CD40 antigen-binding molecule) to a subject prior to administering the nucleic acid construct and the nuclease agent or one or more nucleic acids encoding the nuclease agent.

The terms “initial dose,” “secondary dose(s),” and “tertiary dose(s)” refer to the temporal sequence of administration of the CD40 inhibitor (e.g., CD40 antigen-binding molecule). Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. In some embodiments, the initial, secondary, and tertiary doses may all contain the same amount of the CD40 inhibitor (e.g., CD40 antigen-binding molecule), but may differ from one another in terms of frequency of administration. In some embodiments, the amount of the CD40 inhibitor (e.g., CD40 antigen-binding molecule) contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the CD40 inhibitor (e.g., CD40 antigen-binding molecule). In other embodiments, the initial dose comprises a first amount of the CD40 inhibitor (e.g., CD40 antigen-binding molecule), and the one or more secondary doses each comprise a second amount of the CD40 inhibitor (e.g., CD40 antigen-binding molecule). For example, the first amount of the CD40 inhibitor (e.g., CD40 antigen-binding molecule) can be 1.5×, 2×, 2.5×, 3×, 3.5×, 4×, 5× or more than the second amount of the CD40 inhibitor (e.g., CD40 antigen-binding molecule).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the CD40 inhibitor (e.g., CD40 antigen-binding molecule) molecule that is administered to a patient prior to the administration of the very next dose in the sequence with no intervening doses.

The methods of the disclosure may comprise administering to a patient any number of secondary and/or tertiary doses of a CD40 inhibitor (e.g., CD40 antigen-binding molecule). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1, 2, 3, or 4 weeks after the immediately preceding dose. Similarly, in some embodiments involving multiple tertiary doses, each tertiary dose is administered at the same frequency as the other tertiary doses. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, an amount of a plasma cell depleting agent, e.g., an antigen-binding molecule that binds to B cell maturation antigen (BCMA) and CD3 (e.g., an anti-BCMA×CD3 bispecific antibody), a B cell depleting agent (e.g., anti-CD19 and anti-CD20 antibodies, or a CD20×CD3 antigen-binding molecule (e.g., REGN1979)) (i.e., in combination with a plasma cell depleting agent), an immunoglobulin depleting agent such as a neonatal Fc receptor (FcRn) blocker (e.g., efgartigimod alfa) (i.e., in combination with a plasma cell depleting agent), or an immunogen (e.g., nucleic acid construct, nuclease agent or CRISPR/Cas system, e.g., in an immunogenic delivery vehicle) (e.g., an immunogenic delivery vehicle), or pharmaceutical composition thereof, which is administered to a subject (a subject with preexisting immunity against an immunogen (i.e., an immunogen to be administered to the subject, e.g., an immunogenic delivery vehicle such as, e.g., AAV)) according to the methods disclosed herein is a therapeutically effective amount. In some embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)) (i.e., in combination with a plasma cell depleting agent), the immunoglobulin depleting agent (e.g., efgartigimod alfa) (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle), or pharmaceutical composition thereof, is administered to a subject (a subject with preexisting immunity against an immunogen (i.e., an immunogen to be administered to the subject, e.g., an immunogenic delivery vehicle such as, e.g., AAV)) as a weight-based dose. A “weight-based dose” (e.g., a dose in mg/kg) is a dose of the plasma cell depleting agent, the B cell depleting agent (i.e., in combination with a plasma cell depleting agent), the immunoglobulin depleting agent (i.e., in combination with a plasma cell depleting agent), or the immunogenic delivery vehicle, that will change depending on the subject's weight.

Whenever administration of a plasma cell depleting agent, a B cell depleting agent, or an immunoglobulin depleting agent is discussed in this disclosure, it is in the context of being administered to a subject with preexisting immunity against an immunogen (i.e., an immunogen to be administered to the subject, e.g., an immunogenic delivery vehicle such as, e.g., AAV). Likewise, whenever administration of a B cell depleting agent or an immunoglobulin depleting agent is discussed in this disclosure, it is in the context of being administered in combination with a plasma cell depleting agent.

In other embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)) (i.e., in combination with a plasma cell depleting agent), the immunoglobulin depleting agent (e.g., efgartigimod alfa) (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle), is administered (to a subject with preexisting immunity against an immunogen (i.e., an immunogen to be administered to the subject, e.g., an immunogenic delivery vehicle such as, e.g., AAV)) as a fixed dose. A “fixed dose” (e.g., a dose in mg) means that one dose of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle), is used for all subjects regardless of any specific subject-related factors, such as weight. In one particular embodiment, a fixed dose of a plasma cell depleting agent, a B cell depleting agent (i.e., in combination with a plasma cell depleting agent), an immunoglobulin depleting agent (i.e., in combination with a plasma cell depleting agent), or an immunogen (e.g., nucleic acid construct, nuclease agent or CRISPR/Cas system, e.g., in an immunogenic delivery vehicle), is based on a predetermined weight or age.

Typically, a suitable dose of the plasma cell depleting agent, a B cell depleting agent (i.e., in combination with a plasma cell depleting agent), an immunoglobulin depleting agent (i.e., in combination with a plasma cell depleting agent), and/or the immunogen (e.g., nucleic acid construct, nuclease agent or CRISPR/Cas system, e.g., in an immunogenic delivery vehicle), can be in the range of about 0.001 to about 200.0 milligram per kilogram body weight of the recipient, generally in the range of about 1 to 50 mg per kilogram body weight. For example, the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) dose can be about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure.

In some embodiments, the plasma cell depleting agent (e.g., an anti-BCMA×CD3 bispecific antibody) is administered (to a subject with preexisting immunity against an immunogen (i.e., an immunogen to be administered to the subject, e.g., an immunogenic delivery vehicle such as, e.g., AAV)) at a dose of about 25, about 20 to about 30, about 15 to about 35, about 10 to about 40, about 10 to about 25, about 15 to about 25, about 20 to about 25, about 25 to about 30, about 25 to about 35, or about 25 to about 40 mg/kg. In some embodiments, the plasma cell depleting agent (e.g., an anti-BCMA×CD3 bispecific antibody) is administered at a dose of about 20 to about 30 mg/kg. In some embodiments, the plasma cell depleting agent (e.g., an anti-BCMA×CD3 bispecific antibody) is administered at a dose of about 25 mg/kg.

In some embodiments, the immunoglobulin depleting agent (e.g., FcRn blocker, such as efgartigimod alfa) is administered (to a subject with preexisting immunity against an immunogen (i.e., an immunogen to be administered to the subject, e.g., an immunogenic delivery vehicle such as, e.g., AAV)) at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg (i.e., in combination with a plasma cell depleting agent). In some embodiments, the immunoglobulin depleting agent (e.g., FcRn blocker, such as efgartigimod alfa) is administered at a dose of about 10 to about 20 mg/kg (i.e., in combination with a plasma cell depleting agent). In some embodiments, the immunoglobulin depleting agent (e.g., FcRn blocker, such as efgartigimod alfa) is administered at a dose of about 25 to about 24 mg/kg (i.e., in combination with a plasma cell depleting agent). In some embodiments, the immunoglobulin depleting agent (e.g., FcRn blocker, such as efgartigimod alfa) is administered at a dose of about 20 mg/kg (i.e., in combination with a plasma cell depleting agent). In some embodiments, the immunoglobulin depleting agent (e.g., FcRn blocker, such as efgartigimod alfa) is administered at a dose of about 10 mg/kg (i.e., in combination with a plasma cell depleting agent).

In some embodiments, the immunoglobulin depleting agent (e.g., FcRn blocker, such as efgartigimod alfa) is administered (to a subject with preexisting immunity against an immunogen (i.e., an immunogen to be administered to the subject, e.g., an immunogenic delivery vehicle such as, e.g., AAV)) (i.e., in combination with a plasma cell depleting agent) at a dose of about 10 mg/kg weekly for about 1 week, about 2 weeks, about 3 weeks, about 4 weeks, about 5 weeks, about 6 weeks, about 7 weeks, about 8 weeks, about 9 weeks, or about 10 weeks, or more. In some embodiments, the immunoglobulin depleting agent (e.g., FcRn blocker, such as efgartigimod alfa) is administered (i.e., in combination with a plasma cell depleting agent) at a dose of about 10 mg/kg weekly for about 4 weeks. In various embodiments, such as when a dose of an immunoglobulin depleting agent (e.g., FcRn blocker, such as efgartigimod alfa) is administered (i.e., in combination with a plasma cell depleting agent, and optionally in combination with a B cell depleting agent or an immunogen (e.g., an immunogenic delivery vehicle such as, e.g., AAV), described herein), the first dose of the immunoglobulin depleting agent may be delayed as compared to the first dose of the plasma cell depleting agent, the first dose of the B cell depleting agent, and/or the first dose of the immunogen. In some embodiments, the first dose of the immunoglobulin depleting agent is delayed by about 5 to about 20 days as compared to first dose of the plasma cell depleting agent, the first dose of the B cell depleting agent, and/or the first dose of the immunogen. In some embodiments, the first dose of the immunoglobulin depleting agent is delayed by about 7 to about 15 days as compared to first dose of the plasma cell depleting agent, the first dose of the B cell depleting agent, and/or the first dose of the immunogen. In some embodiments, the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen. In some embodiments, the first dose of the immunoglobulin depleting agent is delayed by about 12 days as compared to first dose of the plasma cell depleting agent, the first dose of the B cell depleting agent, and/or the first dose of the immunogen. In some embodiments, the first dose of the immunoglobulin depleting agent is delayed by about 13 days as compared to first dose of the plasma cell depleting agent, the first dose of the B cell depleting agent, and/or the first dose of the immunogen. In some embodiments, the first dose of the immunoglobulin depleting agent is delayed by about 14 days as compared to first dose of the plasma cell depleting agent, the first dose of the B cell depleting agent, and/or the first dose of the immunogen.

In one specific embodiment, the immunoglobulin depleting agent (e.g., FcRn blocker, such as efgartigimod alfa) is administered (to a subject with preexisting immunity against an immunogen (i.e., an immunogen to be administered to the subject, e.g., an immunogenic delivery vehicle such as, e.g., AAV)) (i.e., in combination with a plasma cell depleting agent) at a dose of about 10 mg/kg weekly for about 4 weeks and the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 9 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen.

In various embodiments, such as when a dose of an immunoglobulin depleting agent (e.g., FcRn blocker, such as efgartigimod alfa) is administered, e.g., in combination with a plasma cell depleting agent (e.g., an anti-BCMA×CD3 bispecific antibody), a B cell depleting agent (e.g., an anti-CD20×CD3 bispecific antibody), and/or an immunogen (i.e., an immunogen to be administered to the subject, e.g., an immunogenic delivery vehicle such as, e.g., AAV)), described herein, the first dose of the immunoglobulin depleting agent may be delayed as compared to the first dose of the plasma cell depleting agent, the first dose of the B cell depleting agent, and/or the first dose of the immunogen. The first dose of the immunoglobulin depleting agent being delayed as compared to the first dose of the plasma cell depleting agent, the first dose of the B cell depleting agent, and/or the first dose of the immunogen may minimize the impact of the immunoglobulin depleting agent (e.g., FcRn blocker, such as efgartigimod alfa) on the antibody drug half-life due to the e.g., FcRn blockade and/or the development of cross-reactive anti-drug antibodies.

In some embodiments, the first dose of the immunoglobulin depleting agent is delayed by about 5 to about 20 days as compared to first dose of the plasma cell depleting agent, the first dose of the B cell depleting agent, and/or the first dose of the immunogen. In some embodiments, the first dose of the immunoglobulin depleting agent is delayed by about 7 to about 15 days as compared to first dose of the plasma cell depleting agent, the first dose of the B cell depleting agent, and/or the first dose of the immunogen. In some embodiments, the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen. In some embodiments, the first dose of the immunoglobulin depleting agent is delayed by about 12 days as compared to first dose of the plasma cell depleting agent, the first dose of the B cell depleting agent, and/or the first dose of the immunogen. In some embodiments, the first dose of the immunoglobulin depleting agent is delayed by about 13 days as compared to first dose of the plasma cell depleting agent, the first dose of the B cell depleting agent, and/or the first dose of the immunogen. In some embodiments, the first dose of the immunoglobulin depleting agent is delayed by about 14 days as compared to first dose of the plasma cell depleting agent, the first dose of the B cell depleting agent, and/or the first dose of the immunogen.

In one specific embodiment, the immunoglobulin depleting agent (e.g., FcRn blocker, such as efgartigimod alfa) is administered at a dose of about 10 mg/kg weekly for about 4 weeks and the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 9 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen.

In various embodiments, such as when a dose of an immunoglobulin depleting agent (e.g., FcRn blocker, such as efgartigimod alfa) is administered, e.g., in combination with an anti-CD40 antibody, described herein, the first dose of the immunoglobulin depleting agent may be delayed as compared to the first dose of the anti-CD40 antibody. The first dose of the immunoglobulin depleting agent being delayed as compared to the first dose of the anti-CD40 antibody may minimize the impact of the immunoglobulin depleting agent (e.g., FcRn blocker, such as efgartigimod alfa) on the antibody drug half-life due to the e.g., FcRn blockade and/or the development of cross-reactive anti-drug antibodies.

In some embodiments, the first dose of the immunoglobulin depleting agent is delayed by about 5 to about 20 days as compared to first dose of the anti-CD40 antibody. In some embodiments, the first dose of the immunoglobulin depleting agent is delayed by about 7 to about 15 days as compared to first dose of the anti-CD40 antibody. In some embodiments, the first dose of the immunoglobulin depleting agent is delayed by about 9 to about 11 days as compared to first dose of the anti-CD40 antibody. In some embodiments, the first dose of the immunoglobulin depleting agent is delayed by about 10 days as compared to first dose of the anti-CD40 antibody. In some embodiments, the first dose of the immunoglobulin depleting agent is delayed by about 11 days as compared to first dose of the anti-CD40 antibody. In some embodiments, the first dose of the immunoglobulin depleting agent is delayed by about 12 days as compared to first dose of the anti-CD40 antibody. In some embodiments, the first dose of the immunoglobulin depleting agent is delayed by about 13 days as compared to first dose of the anti-CD40 antibody. In some embodiments, the first dose of the immunoglobulin depleting agent is delayed by about 14 days as compared to first dose of the anti-CD40 antibody.

In one specific embodiment, the immunoglobulin depleting agent (e.g., FcRn blocker, such as efgartigimod alfa) is administered at a dose of about 10 mg/kg weekly for about 4 weeks and the first dose of the immunoglobulin depleting agent is delayed by about 9 to about 11 days as compared to first dose of the anti-CD40 antibody. In some embodiments, the first dose of the immunoglobulin depleting agent is delayed by about 9 days as compared to first dose of the anti-CD40 antibody. In some embodiments, the first dose of the immunoglobulin depleting agent is delayed by about 10 days as compared to first dose of the anti-CD40 antibody. In some embodiments, the first dose of the immunoglobulin depleting agent is delayed by about 11 days as compared to first dose of the anti-CD40 antibody.

In some embodiments, the B cell depleting agent (e.g., anti-CD19 antibody or anti-CD20 antibody) is administered (to a subject with preexisting immunity against an immunogen (i.e., an immunogen to be administered to the subject, e.g., an immunogenic delivery vehicle such as, e.g., AAV)) at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg (i.e., in combination with a plasma cell depleting agent). In some embodiments, the B cell depleting agent (e.g., anti-CD20×CD3 bispecific antibody or anti-CD19 antibody or anti-CD20 antibody) is administered at a dose of about 10 to about 20 mg/kg (i.e., in combination with a plasma cell depleting agent). In some embodiments, the B cell depleting agent (e.g., anti-CD20×CD3 bispecific antibody or anti-CD19 antibody or anti-CD20 antibody) is administered at a dose of about 25 to about 24 mg/kg (i.e., in combination with a plasma cell depleting agent). In some embodiments, the B cell depleting agent (e.g., anti-CD20×CD3 bispecific antibody or anti-CD19 antibody or anti-CD20 antibody) is administered at a dose of about 20 mg/kg (i.e., in combination with a plasma cell depleting agent).

In some embodiments, the B cell depleting agent (e.g., anti-CD20×CD3 bispecific antibody) is administered (to a subject with preexisting immunity against an immunogen (i.e., an immunogen to be administered to the subject, e.g., an immunogenic delivery vehicle such as, e.g., AAV)) at a dose of about 0.4 to about 0.6, 0.3 to about 0.7, 0.2, to about 0.8, 0.1 to about 0.9, 0.1 to about 0.5, 0.2 to about 0.5, 0.3 to about 0.5, 0.4 to about 0.5, 0.5 to about 0.6, 0.5 to about 0.7, 0.5 to about 0.8, or 0.5 to about 0.9 mg/kg (i.e., in combination with a plasma cell depleting agent). In some embodiments, the B cell depleting agent (e.g., anti-CD19 antibody or anti-CD20 antibody, or anti-CD20×CD3 bispecific antibody) is administered at a dose of about 0.4 to about 0.6 mg/kg (i.e., in combination with a plasma cell depleting agent). In some embodiments, the B cell depleting agent (e.g., anti-CD20×CD3 bispecific antibody) is administered at a dose of about 0.3 to about 0.7 mg/kg (i.e., in combination with a plasma cell depleting agent). In some embodiments, the B cell depleting agent (e.g., anti-CD20×CD3 bispecific antibody) is administered at a dose of about 0.5 mg/kg (i.e., in combination with a plasma cell depleting agent). In some embodiments, the B cell depleting agent (e.g., anti-CD20×CD3 bispecific antibody) is administered at a dose of about 0.1 mg/kg (i.e., in combination with a plasma cell depleting agent). In some embodiments, the B cell depleting agent (e.g., anti-CD20×CD3 bispecific antibody) is administered at a dose of about 1 mg/kg (i.e., in combination with a plasma cell depleting agent).

In some embodiments, the plasma cell depleting agent, the B cell depleting agent (i.e., in combination with a plasma cell depleting agent), the immunoglobulin depleting agent (i.e., in combination with a plasma cell depleting agent), or optionally the immunogen (e.g., nucleic acid construct, nuclease agent or CRISPR/Cas system, e.g., in an immunogenic delivery vehicle) is administered (to a subject with preexisting immunity against an immunogen (i.e., an immunogen to be administered to the subject, e.g., an immunogenic delivery vehicle such as, e.g., AAV)) as a fixed dose of between about 5 mg to about 2500 mg. In some embodiments, the plasma cell depleting agent, the B cell depleting agent (i.e., in combination with a plasma cell depleting agent), or the immunoglobulin depleting agent (i.e., in combination with a plasma cell depleting agent) is administered as a fixed dose of about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

In one embodiment, for a plasma cell depleting agent (e.g., an anti-BCMA/anti-CD3 bispecific antibody), a therapeutically effective amount can be from about 0.05 mg to about 500 mg, from about 1 mg to about 500 mg, from about 10 mg to about 450 mg, from about 50 mg to about 400 mg, from about 75 mg to about 350 mg, or from about 100 mg to about 300 mg of the antibody. For example, in various embodiments, the amount of the plasma cell depleting agent is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, of the plasma cell depleting agent.

In some embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)) (i.e., in combination with a plasma cell depleting agent), the immunoglobulin depleting agent (e.g., efgartigimod alfa) (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle) is administered to a subject (to a subject with preexisting immunity against an immunogen (i.e., an immunogen to be administered to the subject, e.g., an immunogenic delivery vehicle such as, e.g., AAV)) at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogenic delivery vehicle can be administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogen (e.g., nucleic acid construct, nuclease agent or CRISPR/Cas system, e.g., in an immunogenic delivery vehicle) (e.g., an immunogenic delivery vehicle) can be administered at a dosing frequency of about four times a year, twice a year, once a week, once every two years, once every three years, once every four years, once every five years, once every six years, once every eight years, once every twelve years, or less frequently so long as a therapeutic response is achieved.

Dose ranges and frequency of administration of an immunogen (e.g., nucleic acid construct, nuclease agent or CRISPR/Cas system, e.g., in an immunogenic delivery vehicle), e.g., an immunogenic delivery vehicle such as a vector (e.g., a viral vector such as an AAV vector) described herein can vary depending on the nature of, and/or the medical condition, as well as parameters of a specific subject and the route of administration used. As a non-limiting example, vector compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the vector compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

In some embodiments, multiple doses of a plasma cell depleting agent (e.g., an anti-BCMA×CD3 bispecific antibody), a B cell depleting agent (e.g., anti-CD19/CD20 antibodies, or a CD20×CD3 antigen-binding molecule (e.g., REGN1979)) (i.e., in combination with a plasma cell depleting agent), an immunoglobulin depleting agent (e.g., efgartigimod alfa) (i.e., in combination with a plasma cell depleting agent), or an immunogen (e.g., nucleic acid construct, nuclease agent or CRISPR/Cas system, e.g., in an immunogenic delivery vehicle) (e.g., an immunogenic delivery vehicle) are administered to a subject (to a subject with preexisting immunity against an immunogen (i.e., an immunogen to be administered to the subject, e.g., an immunogenic delivery vehicle such as, e.g., AAV)) over a defined time course. In some embodiments, the methods of the present disclosure comprise sequentially administering to a subject (to a subject with preexisting immunity against an immunogen (i.e., an immunogen to be administered to the subject, e.g., an immunogenic delivery vehicle such as, e.g., AAV)) multiple doses of the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)) (i.e., in combination with a plasma cell depleting agent), the immunoglobulin depleting agent (e.g., efgartigimod alfa) (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle).

In some embodiments, the immunogen (e.g., nucleic acid construct, nuclease agent or CRISPR/Cas system, e.g., in an immunogenic delivery vehicle) (e.g., an immunogenic delivery vehicle such as a vector, e.g., an AAV vector) may be administered in accordance with a repeat dosing regimen wherein the immunogen (e.g., nucleic acid construct, nuclease agent or CRISPR/Cas system, e.g., in an immunogenic delivery vehicle) (e.g., an immunogenic delivery vehicle) may be administered a first time (e.g., in an initial dose) and then re-administered any number of subsequent times thereafter at any amount over the time course of treatment of a subject. For example, the immunogen (e.g., nucleic acid construct, nuclease agent or CRISPR/Cas system, e.g., in an immunogenic delivery vehicle) may be re-administered once, twice, three times, four times, five times, six times, seven times, eight times, nine times, ten times, or more, over the time course of the treatment of a subject which can occur over any number of days, weeks, or years. In some embodiments, when the immunogen (e.g., nucleic acid construct, nuclease agent or CRISPR/Cas system, e.g., in an immunogenic delivery vehicle) (e.g., an immunogenic delivery vehicle) comprises a vector, e.g., a viral vector such as an AAV vector, the vector which is administered first in a repeat dosing regimen may comprise the same vector which is re-administered second in the repeat dosing regimen, or any number of subsequent times thereafter. In some embodiments, when the immunogen (e.g., nucleic acid construct, nuclease agent or CRISPR/Cas system, e.g., in an immunogenic delivery vehicle) (e.g., an immunogenic delivery vehicle) comprises a vector, e.g., a viral vector such as an AAV vector, the vector which is administered first in a repeat dosing regimen may comprise a different vector than is re-administered second in the repeat dosing regimen, or any number of subsequent times thereafter.

In some embodiments, immunogen (e.g., nucleic acid construct, nuclease agent or CRISPR/Cas system, e.g., in an immunogenic delivery vehicle) (e.g., an immunogenic delivery vehicle), e.g., a viral vector such as an AAV vector, may be administered in accordance with a stepwise dosing regimen. Stepwise dosing of an immunogen (e.g., nucleic acid construct, nuclease agent or CRISPR/Cas system, e.g., in an immunogenic delivery vehicle) can refer to breaking up (i.e., dividing) dosing of the same immunogen (e.g., nucleic acid construct, nuclease agent or CRISPR/Cas system, e.g., in an immunogenic delivery vehicle) over multiple administrations. In some embodiments, the dosing of the same immunogen (e.g., nucleic acid construct, nuclease agent or CRISPR/Cas system, e.g., in an immunogenic delivery vehicle) is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of an immunogen (e.g., nucleic acid construct, nuclease agent or CRISPR/Cas system, e.g., in an immunogenic delivery vehicle), e.g., an immunogenic delivery vehicle, e.g., a viral vector such as an AAV vector, the stepwise dosing regimen may result in a gradual increase in therapeutic transgene levels with each administration of the immunogen (e.g., nucleic acid construct, nuclease agent or CRISPR/Cas system, e.g., in an immunogenic delivery vehicle). Without wishing to be bound by theory, a stepwise dosing regimen used in the administration of an immunogen (e.g., nucleic acid construct, nuclease agent or CRISPR/Cas system, e.g., in an immunogenic delivery vehicle) comprising an immunogenic delivery vehicle, e.g., a viral vector such as an AAV vector, can result in enhanced control over transgene expression in a cell and/or subject, since for some transgenes too much expression can result in its own pathology.

In some embodiments, an immunogen (e.g., nucleic acid construct, nuclease agent or CRISPR/Cas system, e.g., in an immunogenic delivery vehicle) (e.g., an immunogenic delivery vehicle) is administered to a subject in combination with a plasma cell depleting agent (optionally in combination with a B cell depleting agent and/or an immunoglobulin depleting agent) (i.e., to subjects with preexisting immunity against an immunogen (i.e., an immunogen to be administered to the subject, e.g., an immunogenic delivery vehicle such as, e.g., AAV)). The plasma cell depleting agent (optionally in combination with the B cell depleting agent and/or the immunoglobulin depleting agent) can be administered prior to, simultaneously with, or after the immunogen (e.g., nucleic acid construct, nuclease agent or CRISPR/Cas system, e.g., in an immunogenic delivery vehicle). In one example, the plasma cell depleting agent (optionally in combination with the B cell depleting agent and/or the immunoglobulin depleting agent) is administered prior to the immunogen (e.g., nucleic acid construct, nuclease agent or CRISPR/Cas system, e.g., in an immunogenic delivery vehicle). In another example, the plasma cell depleting agent (optionally in combination with the B cell depleting agent and/or the immunoglobulin depleting agent) is administered prior to and after the immunogen (e.g., nucleic acid construct, nuclease agent or CRISPR/Cas system, e.g., in an immunogenic delivery vehicle). In another example, the plasma cell depleting agent (optionally in combination with the B cell depleting agent and/or the immunoglobulin depleting agent) is administered simultaneously with the immunogen (e.g., nucleic acid construct, nuclease agent or CRISPR/Cas system, e.g., in an immunogenic delivery vehicle). In some embodiments, the plasma cell depleting agent is administered before the administration of the immunogen. In some embodiments, such as when the immunogen is administered two or more times, the plasma cell depleting agent is administered before and/or between each of the administrations of the immunogen. In some embodiments, the plasma cell depleting agent is administered again within a short period of the first administration. In some embodiments, the plasma cell depleting agent is continuously administered throughout the pre-dose and re-dose periods (e.g., to clear plasma cells and keep plasma cell levels low).

In some embodiments, when the plasma cell depleting agent (optionally in combination with the B cell depleting agent and/or the immunoglobulin depleting agent) is administered prior to, simultaneously with, and/or after the immunogen (e.g., nucleic acid construct, nuclease agent or CRISPR/Cas system, e.g., in an immunogenic delivery vehicle) (e.g., an immunogenic delivery vehicle), the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) once, twice, three times, four times, five times, six times, seven times, eight times, nine times, ten times, eleven times, twelve times, thirteen times, fourteen times, fifteen times, sixteen times, seventeen times, eighteen times, nineteen times, or twenty times or more, prior to, simultaneously with, and/or after the administration of the immunogen (e.g., nucleic acid construct, nuclease agent or CRISPR/Cas system, e.g., in an immunogenic delivery vehicle) (e.g., an immunogenic delivery vehicle). In some embodiments, the CD40 antigen-binding molecule and/or the CD40 inhibitor is administered seven days prior to the administration of the immunogen (e.g., nucleic acid construct, nuclease agent or CRISPR/Cas system, e.g., in an immunogenic delivery vehicle) (e.g., an immunogenic delivery vehicle). In some embodiments, the CD40 antigen-binding molecule and/or the CD40 inhibitor is administered three days prior to the administration of the immunogen (e.g., nucleic acid construct, nuclease agent or CRISPR/Cas system, e.g., in an immunogenic delivery vehicle) (e.g., an immunogenic delivery vehicle). In some embodiments, the CD40 antigen-binding molecule and/or the CD40 inhibitor is administered the same day as the administration of the immunogen (e.g., nucleic acid construct, nuclease agent or CRISPR/Cas system, e.g., in an immunogenic delivery vehicle) (e.g., an immunogenic delivery vehicle). In some embodiments, the CD40 antigen-binding molecule and/or the CD40 inhibitor is administered one day after the administration of the immunogen (e.g., nucleic acid construct, nuclease agent or CRISPR/Cas system, e.g., in an immunogenic delivery vehicle) (e.g., an immunogenic delivery vehicle). In some embodiments, when an immunogen (e.g., nucleic acid construct, nuclease agent or CRISPR/Cas system, e.g., in an immunogenic delivery vehicle) is administered in accordance with a repeat dosing regimen, a plasma cell depleting agent (optionally in combination with a B cell depleting agent and/or an immunoglobulin depleting agent) may be administered any number of times prior to, simultaneously with, and/or after a first and/or second administration of the immunogen (e.g., nucleic acid construct, nuclease agent or CRISPR/Cas system, e.g., in an immunogenic delivery vehicle), and/or any number of subsequent administrations of the immunogen thereafter. Without wishing to be bound by theory, when a plasma cell depleting agent (optionally in combination with a B cell depleting agent and/or an immunoglobulin depleting agent) is administered to inhibit an immune response to an immunogen (e.g., nucleic acid construct, nuclease agent or CRISPR/Cas system, e.g., in an immunogenic delivery vehicle) in a subject in need thereof (to a subject with preexisting immunity against an immunogen (i.e., an immunogen to be administered to the subject, e.g., an immunogenic delivery vehicle such as, e.g., AAV)), e.g., an anti-drug antibody response to an immunogenic protein, the plasma cell depleting agent (optionally in combination with the B cell depleting agent and/or the immunoglobulin depleting agent) may be co-administered (e.g., administered prior to, simultaneously with, and/or after the immunogen) to prevent the response of the immune system of the subject on each dose of the immunogen (e.g., nucleic acid construct, nuclease agent or CRISPR/Cas system, e.g., in an immunogenic delivery vehicle). As an example, an immunogen comprising a bacterial IgG cleaving enzyme IdeS/imlifidase may be administered to a subject for overcoming AAV pre-existing immunity; however, IdeS itself is immunogenic and can only be administered once. Co-administration of a plasma cell depleting agent (optionally in combination with a B cell depleting agent and/or an immunoglobulin depleting agent) described herein with IdeS/imlifidase can prevent the de novo response to IdeS protein.

According to certain embodiments of the present disclosure, a plasma cell depleting agent (optionally in combination with a B cell depleting agent and/or an immunoglobulin depleting agent) may be administered to a subject (to a subject with preexisting immunity against an immunogen (i.e., an immunogen to be administered to the subject, e.g., an immunogenic delivery vehicle such as, e.g., AAV)) separately from an immunogen (e.g., nucleic acid construct, nuclease agent or CRISPR/Cas system, e.g., in an immunogenic delivery vehicle) (e.g., an immunogenic delivery vehicle) described herein.

In some embodiments, when a plasma cell depleting agent (optionally in combination with a B cell depleting agent and/or an immunoglobulin depleting agent) and an immunogen (e.g., nucleic acid construct, nuclease agent or CRISPR/Cas system, e.g., in an immunogenic delivery vehicle) (e.g., an immunogenic delivery vehicle) are administered separately, the plasma cell depleting agent (optionally in combination with the B cell depleting agent and/or the immunoglobulin depleting agent) may be administered (to a subject with preexisting immunity against an immunogen (i.e., an immunogen to be administered to the subject, e.g., an immunogenic delivery vehicle such as, e.g., AAV)) simultaneously with the administration of the immunogen (e.g., nucleic acid construct, nuclease agent or CRISPR/Cas system, e.g., in an immunogenic delivery vehicle). In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) one or more times during the administration of the immunogen (e.g., nucleic acid construct, nuclease agent or CRISPR/Cas system, e.g., in an immunogenic delivery vehicle). In some embodiments, the immunogen (e.g., nucleic acid construct, nuclease agent or CRISPR/Cas system, e.g., in an immunogenic delivery vehicle) is administered one or more times during the administration of the plasma cell depleting agent (optionally in combination with the B cell depleting agent and/or the immunoglobulin depleting agent) (the plasma cell depleting agent is administered to a subject with preexisting immunity against an immunogen (i.e., an immunogen to be administered to the subject, e.g., an immunogenic delivery vehicle such as, e.g., AAV)). In some embodiments, the plasma cell depleting agent is administered (to a subject with preexisting immunity against an immunogen (i.e., an immunogen to be administered to the subject, e.g., an immunogenic delivery vehicle such as, e.g., AAV)) before the administration of the immunogen. In some embodiments, such as when the immunogen is administered two or more times, the plasma cell depleting agent is administered to a subject with preexisting immunity against an immunogen (i.e., an immunogen to be administered to the subject, e.g., an immunogenic delivery vehicle such as, e.g., AAV) before and/or between each of the administrations of the immunogen (e.g., an immunogenic delivery vehicle such as, e.g., AAV). In some embodiments, the plasma cell depleting agent is administered (to a subject with preexisting immunity against an immunogen (i.e., an immunogen to be administered to the subject, e.g., an immunogenic delivery vehicle such as, e.g., AAV)) again within a short period of the first administration. In some embodiments, the plasma cell depleting agent is continuously administered (to a subject with preexisting immunity against an immunogen (i.e., an immunogen to be administered to the subject, e.g., an immunogenic delivery vehicle such as, e.g., AAV)) throughout the pre-dose and re-dose periods (e.g., to clear plasma cells and keep plasma cell levels low).

In some embodiments, a B cell depleting agent is administered (to a subject with preexisting immunity against an immunogen (i.e., an immunogen to be administered to the subject, e.g., an immunogenic delivery vehicle such as, e.g., AAV)) prior to a plasma cell depleting agent. In some embodiments, a B cell depleting agent is administered (to a subject with preexisting immunity against an immunogen (i.e., an immunogen to be administered to the subject, e.g., an immunogenic delivery vehicle such as, e.g., AAV)) simultaneously with a plasma cell depleting agent. In some embodiments, a B cell depleting agent is administered (to a subject with preexisting immunity against an immunogen (i.e., an immunogen to be administered to the subject, e.g., an immunogenic delivery vehicle such as, e.g., AAV)) subsequent to a plasma cell depleting agent. In some embodiments, a B cell depleting agent is administered (to a subject with preexisting immunity against an immunogen (i.e., an immunogen to be administered to the subject, e.g., an immunogenic delivery vehicle such as, e.g., AAV)) prior to and subsequent to a plasma cell depleting agent. In some embodiments, a B cell depleting agent is administered (to a subject with preexisting immunity against an immunogen (i.e., an immunogen to be administered to the subject, e.g., an immunogenic delivery vehicle such as, e.g., AAV)) prior to and simultaneously with a plasma cell depleting agent. In some embodiments, a B cell depleting agent is administered (to a subject with preexisting immunity against an immunogen (i.e., an immunogen to be administered to the subject, e.g., an immunogenic delivery vehicle such as, e.g., AAV)) simultaneously with and subsequent to a plasma cell depleting agent. In theory, B cell depletion in a subject with preexisting immunity against an immunogen (i.e., an immunogen to be administered to the subject, e.g., an immunogenic delivery vehicle such as, e.g., AAV) could be conducted before or after plasma cell depletion with the same effect, provided that B cells remain depleted up until the time of dosing with the immunogen.

In some embodiments, an immunoglobulin depleting agent is administered (to a subject with preexisting immunity against an immunogen (i.e., an immunogen to be administered to the subject, e.g., an immunogenic delivery vehicle such as, e.g., AAV)) subsequent to a plasma cell depleting agent. In some embodiments, an immunoglobulin depleting agent is administered (to a subject with preexisting immunity against an immunogen (i.e., an immunogen to be administered to the subject, e.g., an immunogenic delivery vehicle such as, e.g., AAV)) subsequent to a B cell depleting agent. In some embodiments, an immunoglobulin depleting agent is administered (to a subject with preexisting immunity against an immunogen (i.e., an immunogen to be administered to the subject, e.g., an immunogenic delivery vehicle such as, e.g., AAV)) subsequent to a plasma cell depleting agent and a B cell depleting agent. For example, if the plasma cell depleting agent is an anti-BCMA×CD3 bispecific antibody, administering the immunoglobulin depleting agent after the plasma cell depleting agent (to a subject with preexisting immunity against an immunogen (i.e., an immunogen to be administered to the subject, e.g., an immunogenic delivery vehicle such as, e.g., AAV)) will prevent more rapid clearance of the plasma cell depleting agent. For example, if the B cell depleting agent is an anti-CD20×CD3 bispecific antibody, administering the immunoglobulin depleting agent after the B cell depleting agent (to a subject with preexisting immunity against an immunogen (i.e., an immunogen to be administered to the subject, e.g., an immunogenic delivery vehicle such as, e.g., AAV)) will prevent more rapid clearance of the B cell depleting agent. The timing can be affected by what immunoglobulin depleting agent is used. For example, different treatment regimens would be expected for FcRn blockers vs. IgG degrading enzymes (e.g., IdeS). IdeS is an enzyme and therefore acts much more rapidly than FcRn blockade, clearing IgGs within hours to days. For FcRn blockade, several weeks of treatment may be required to fully clear anti-AAV IgGs from circulation.

In some embodiments, a plasma cell depleting agent (optionally in combination with a B cell depleting agent and/or an immunoglobulin depleting agent) and an immunogen (e.g., an immunogenic delivery vehicle) may be administered (i.e., the plasma cell depleting agent is administered to a subject with preexisting immunity against an immunogen (i.e., an immunogen to be administered to the subject, e.g., an immunogenic delivery vehicle such as, e.g., AAV)) separately over a defined time course. In certain embodiments, multiple doses of a plasma cell depleting agent (optionally in combination with a B cell depleting agent and/or an immunoglobulin depleting agent) and/or an immunogen (e.g., nucleic acid construct, nuclease agent or CRISPR/Cas system, e.g., in an immunogenic delivery vehicle) (e.g., an immunogenic delivery vehicle) described herein may be administered to a subject (i.e., the plasma cell depleting agent is administered a subject with preexisting immunity against an immunogen (i.e., an immunogen to be administered to the subject, e.g., an immunogenic delivery vehicle such as, e.g., AAV)) over a defined time course. The methods according to such aspects of the disclosure may comprise sequentially administering to a subject multiple doses of a plasma cell depleting agent (optionally in combination with a B cell depleting agent and/or an immunoglobulin depleting agent) (to a subject with preexisting immunity against an immunogen (i.e., an immunogen to be administered to the subject, e.g., an immunogenic delivery vehicle such as, e.g., AAV)) and/or immunogen (e.g., nucleic acid construct, nuclease agent or CRISPR/Cas system, e.g., in an immunogenic delivery vehicle) of the disclosure. In some embodiments, when the plasma cell depleting agent (optionally in combination with the B cell depleting agent and/or the immunoglobulin depleting agent) and immunogen (e.g., nucleic acid construct, nuclease agent or CRISPR/Cas system, e.g., in an immunogenic delivery vehicle) are administered sequentially, the plasma cell depleting agent (optionally in combination with the B cell depleting agent and/or the immunoglobulin depleting agent) can be administered (to a subject with preexisting immunity against an immunogen (i.e., an immunogen to be administered to the subject, e.g., an immunogenic delivery vehicle such as, e.g., AAV)) before and/or in between each of the administrations of the immunogen(s) (e.g., nucleic acid construct, nuclease agent or CRISPR/Cas system, e.g., in an immunogenic delivery vehicle). As used herein, “sequentially administering” means that each dose of the plasma cell depleting agent (optionally in combination with the B cell depleting agent and/or the immunoglobulin depleting agent), and/or the immunogenic delivery vehicle, is administered to the subject at a different point in time, e.g., on different days separated by a predetermined interval (e.g., hours, days, weeks, months, or years). In some embodiments, the methods of the disclosure comprise sequentially administering to the subject (to a subject with preexisting immunity against an immunogen (i.e., an immunogen to be administered to the subject, e.g., an immunogenic delivery vehicle such as, e.g., AAV)) a single initial dose of the plasma cell depleting agent (optionally in combination with the B cell depleting agent and/or the immunoglobulin depleting agent) and/or the immunogen (e.g., nucleic acid construct, nuclease agent or CRISPR/Cas system, e.g., in an immunogenic delivery vehicle), followed by one or more secondary doses of the plasma cell depleting agent (optionally in combination with the B cell depleting agent and/or the immunoglobulin depleting agent) (to a subject with preexisting immunity against an immunogen (i.e., an immunogen to be administered to the subject, e.g., an immunogenic delivery vehicle such as, e.g., AAV)) and/or the immunogen (e.g., nucleic acid construct, nuclease agent or CRISPR/Cas system, e.g., in an immunogenic delivery vehicle) (e.g., an immunogenic delivery vehicle), and optionally followed by one or more tertiary doses of the plasma cell depleting agent (optionally in combination with the B cell depleting agent and/or the immunoglobulin depleting agent) (to a subject with preexisting immunity against an immunogen (i.e., an immunogen to be administered to the subject, e.g., an immunogenic delivery vehicle such as, e.g., AAV)) and/or the immunogen (e.g., nucleic acid construct, nuclease agent or CRISPR/Cas system, e.g., in an immunogenic delivery vehicle).

The terms “initial dose,” “secondary dose(s),” and “tertiary dose(s)” refer to the temporal sequence of administration of the plasma cell depleting agent (optionally in combination with the B cell depleting agent and/or the immunoglobulin depleting agent) (to a subject with preexisting immunity against an immunogen (i.e., an immunogen to be administered to the subject, e.g., an immunogenic delivery vehicle such as, e.g., AAV)) and/or the immunogen (e.g., nucleic acid construct, nuclease agent or CRISPR/Cas system, e.g., in an immunogenic delivery vehicle) (e.g., an immunogenic delivery vehicle). Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. In some embodiments, the initial, secondary, and tertiary doses may all contain the same amount of the plasma cell depleting agent, the B cell depleting agent (i.e., in combination with the plasma cell depleting agent), the immunoglobulin depleting agent (i.e., in combination with the plasma cell depleting agent), and/or the immunogen (e.g., nucleic acid construct, nuclease agent or CRISPR/Cas system, e.g., in an immunogenic delivery vehicle) but may differ from one another in terms of frequency of administration. In some embodiments, the amount of the plasma cell depleting agent, B cell depleting agent (i.e., in combination with a plasma cell depleting agent), immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle). In other embodiments, the initial dose comprises a first amount of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle) and the one or more secondary doses each comprise a second amount of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle). For example, the first amount of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle) can be 1.5×, 2×, 2.5×, 3×, 3.5×, 4×, 5× or more than the second amount of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 1312, 14, 1412, or more) weeks after the immediately preceding dose. The phrase “the immediately preceding dose,” as used herein, means, in a sequence of multiple administrations, the dose of the plasma cell depleting agent, the B cell depleting agent (i.e., in combination with a plasma cell depleting agent), the immunoglobulin depleting agent (i.e., in combination with a plasma cell depleting agent), or the immunogen (e.g., nucleic acid construct, nuclease agent or CRISPR/Cas system, e.g., in an immunogenic delivery vehicle) (e.g., an immunogenic delivery vehicle) that is administered to a subject (to a subject with preexisting immunity against an immunogen (i.e., an immunogen to be administered to the subject, e.g., an immunogenic delivery vehicle such as, e.g., AAV)) prior to the administration of the very next dose in the sequence with no intervening doses.

The methods of the disclosure may comprise administering to a subject (to a subject with preexisting immunity against an immunogen (i.e., an immunogen to be administered to the subject, e.g., an immunogenic delivery vehicle such as, e.g., AAV)) any number of secondary and/or tertiary doses of a plasma cell depleting agent, a B cell depleting agent (i.e., in combination with a plasma cell depleting agent), an immunoglobulin depleting agent (i.e., in combination with a plasma cell depleting agent), or an immunogen (e.g., nucleic acid construct, nuclease agent or CRISPR/Cas system, e.g., in an immunogenic delivery vehicle) (e.g., an immunogenic delivery vehicle). For example, in certain embodiments, only a single secondary dose is administered to the subject. In other embodiments, two or more (e.g., 2, 3, 4, 5, 6, 7, 8, or more) secondary doses are administered to the subject. Likewise, in certain embodiments, only a single tertiary dose is administered to the subject. In other embodiments, two or more (e.g., 2, 3, 4, 5, 6, 7, 8, or more) tertiary doses are administered to the subject.

In some embodiments involving multiple secondary doses, each secondary dose is administered at the same frequency as the other secondary doses. For example, each secondary dose may be administered to the subject 1, 2, 3, or 4 weeks after the immediately preceding dose. Similarly, in some embodiments involving multiple tertiary doses, each tertiary dose is administered at the same frequency as the other tertiary doses. Alternatively, the frequency at which the secondary and/or tertiary doses are administered to a subject can vary over the course of the treatment regimen. The frequency of administration may also be adjusted during the course of treatment by a physician depending on the needs of the individual subject following clinical examination.

In some embodiments, the secondary and/or tertiary doses of an immunogen (e.g., nucleic acid construct, nuclease agent or CRISPR/Cas system, e.g., in an immunogenic delivery vehicle) (e.g., an immunogenic delivery vehicle) comprising a viral particle or vector (e.g., a viral vector such as an AAV vector) administered to the subject is of the same or similar viral origin as the initial dose. In some embodiments, the secondary and/or tertiary doses of an immunogen (e.g., nucleic acid construct, nuclease agent or CRISPR/Cas system, e.g., in an immunogenic delivery vehicle) (e.g., an immunogenic delivery vehicle) comprising a viral particle or vector administered to the subject is of a different viral origin then the initial dose.

In some embodiments, the subsequently administered viral vector is administered via the same administration route as the originally administered viral vector. In some embodiments, the subsequently administered viral vector is administered via a different administration route from the originally administered viral vector.

In some embodiments, when a plasma cell depleting agent (optionally in combination with the B cell depleting agent and/or the immunoglobulin depleting agent) and/or an immunogen (e.g., nucleic acid construct, nuclease agent or CRISPR/Cas system, e.g., in an immunogenic delivery vehicle) (e.g., an immunogenic delivery vehicle) herein are sequentially administered, the immunogen (e.g., nucleic acid construct, nuclease agent or CRISPR/Cas system, e.g., in an immunogenic delivery vehicle) (e.g., an immunogenic delivery vehicle) may be administered as a first component of the dosing regimen and the plasma cell depleting agent (optionally in combination with the B cell depleting agent and/or the immunoglobulin depleting agent) may be administered (to a subject with preexisting immunity against an immunogen (i.e., an immunogen to be administered to the subject, e.g., an immunogenic delivery vehicle such as, e.g., AAV)) as a second component of the dosing regimen (i.e., the immunogen (e.g., nucleic acid construct, nuclease agent or CRISPR/Cas system, e.g., in an immunogenic delivery vehicle) may be administered before the plasma cell depleting agent (optionally in combination with the B cell depleting agent and/or the immunoglobulin depleting agent)). In some embodiments, the immunogen (e.g., nucleic acid construct, nuclease agent or CRISPR/Cas system, e.g., in an immunogenic delivery vehicle) may be administered as a second component of the dosing regimen and the plasma cell depleting agent (optionally in combination with the B cell depleting agent and/or the immunoglobulin depleting agent) may be administered (to a subject with preexisting immunity against an immunogen (i.e., an immunogen to be administered to the subject, e.g., an immunogenic delivery vehicle such as, e.g., AAV)) as a first component of a dosing regimen (i.e., the immunogen (e.g., nucleic acid construct, nuclease agent or CRISPR/Cas system, e.g., in an immunogenic delivery vehicle) may be administered after the plasma cell depleting agent (optionally in combination with the B cell depleting agent and/or the immunoglobulin depleting agent)). In some embodiments, an immunogen (e.g., nucleic acid construct, nuclease agent or CRISPR/Cas system, e.g., in an immunogenic delivery vehicle) and a plasma cell depleting agent (optionally in combination with the B cell depleting agent and/or the immunoglobulin depleting agent) may be sequentially administered, in either of the above-described orders, with variable time intervals between administration. For example, the time interval between administration of the immunogen (e.g., nucleic acid construct, nuclease agent or CRISPR/Cas system, e.g., in an immunogenic delivery vehicle) (e.g., an immunogenic delivery vehicle) and the plasma cell depleting agent (optionally in combination with the B cell depleting agent and/or the immunoglobulin depleting agent) may be at least about 30 seconds, at least about 35 seconds, at least about 40 seconds, at least about 45 seconds, at least about 50 seconds, at least about 55 seconds, at least about 1 minute, at least about 2 minutes, at least about 5 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 10 to 12 hours, at least about 12 to 14 hours, at least about 14 to 16 hours, at least about 16 to 18 hours, at least about 18 to 20 hours, at least about 20 to 22 hours, at least about 22 to 24 hours, at least about 1 day, at least about 2 days, at least about 3 days, at least about 4 days, at least about 5 days, at least about 6 days, at least about 7 days, at least about 8 days, at least about 9 days, at least about 10 days, at least about 10 to 12 days, at least about 12 to 14 days, at least about 14 to 16 days, at least about 16 to 18 days, at least about 18 to 20 days, at least about 20 to 22 days, at least about 22 to 24 days, at least about 24 to 26 days, at least about 28 days, at least about 29 days, at least about 30 days, at least about 31 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, or more.

Any of the above methods can further comprise any of various subsequent administration steps described herein. The subsequent administration step can comprise, for example, administering the plasma cell depleting agent (optionally in combination with the B cell depleting agent and/or the immunoglobulin depleting agent) (to a subject with preexisting immunity against an immunogen (i.e., an immunogen to be administered to the subject, e.g., an immunogenic delivery vehicle such as, e.g., AAV)) and an immunogen (e.g., nucleic acid construct, nuclease agent or CRISPR/Cas system, e.g., in an immunogenic delivery vehicle) (e.g., an immunogenic delivery vehicle) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can comprise, for example, administering a second immunogen (e.g., nucleic acid construct, nuclease agent or CRISPR/Cas system, e.g., in an immunogenic delivery vehicle) (e.g., an immunogenic delivery vehicle), e.g. an immune delivery vehicle comprising, e.g., a vector comprising a coding sequence for a second polypeptide of interest (e.g., that is different from a first polypeptide of interest encoded by a first vector administered in an initial administration step) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some embodiments, the present disclosure provides a method for increasing effectiveness of a subsequently administered viral vector following an originally administered viral vector in a subject in need thereof, comprising administering to the subject an effective amount of a plasma cell depleting agent (optionally in combination with a B cell depleting agent and/or an immunoglobulin depleting agent) (to a subject with preexisting immunity against an immunogen (i.e., an immunogen to be administered to the subject, e.g., an immunogenic delivery vehicle such as, e.g., AAV)), and the subsequently administered viral vector is of the same or similar viral origin as the originally administered viral vector. In some embodiments, provided herein is a method for increasing effectiveness of a subsequently administered viral vector following an originally administered viral vector in a subject in need thereof, the method comprising administering to the subject an effective amount of a CD40 inhibitor, wherein the subsequently administered viral vector is of the same or similar viral origin as the originally administered viral vector. In some embodiments, the subject does not have a pre-existing immunity against the viral vectors. In some embodiments, the subject does not have a pre-existing immunity against the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent. In some embodiments, the present disclosure contemplates a method for increasing effectiveness of a subsequently administered viral vector following an originally administered viral vector in a subject in need thereof with preexisting immunity, comprising administering to the subject an effective amount of a plasma cell depleting agent, optionally in combination with a B cell depleting agent and/or an immunoglobulin depleting agent, and the subsequently administered viral vector is of the same or similar viral origin as the originally administered viral vector.

In some embodiments, the subsequently administered viral vector is administered via the same administration route as the originally administered viral vector.

In some embodiments, the subsequently administered viral vector is administered via a different administration route from the originally administered viral vector.

In some embodiments, the CD40 inhibitor is administered before the administration of the viral vector(s). In some embodiments, the CD40 inhibitor is administered before the originally administered viral vector to the subject. In some embodiments, the CD40 inhibitor is administered simultaneously with the administration of the viral vector(s). In some embodiments, the CD40 inhibitor is administered simultaneously with the administration of the originally administered viral vector and/or subsequently administered viral vector to the subject. In some embodiments, the CD40 inhibitor is administered after the administration of the viral vector(s). In some embodiments, the CD40 inhibitor is administered after the administration of the originally administered viral vector but before administering the subsequently administered viral vector to the subject. In some embodiments, the CD40 inhibitor is administered after the administration of the subsequently administered viral vector to the subject. In some embodiments, the viral vectors are administered two or more times and the CD40 inhibitor is administered before and/or between each of the administrations of the viral vectors.

In some embodiments, the plasma cell depleting agent (optionally in combination with the B cell depleting agent and/or the immunoglobulin depleting agent) is administered before the administration of the subsequently administered viral vector(s) (to a subject with preexisting immunity against an immunogen (i.e., an immunogen to be administered to the subject, e.g., an immunogenic delivery vehicle such as, e.g., AAV)).

In some embodiments, the plasma cell depleting agent (optionally in combination with the B cell depleting agent and/or the immunoglobulin depleting agent) is administered simultaneously with the administration of the subsequently administered viral vector(s) (to a subject with preexisting immunity against an immunogen (i.e., an immunogen to be administered to the subject, e.g., an immunogenic delivery vehicle such as, e.g., AAV)).

In some embodiments, the subsequently administered viral vectors are administered two or more times and the plasma cell depleting agent (optionally in combination with the B cell depleting agent and/or the immunoglobulin depleting agent) is administered before and/or between each of the administrations of the subsequently administered viral vectors (to a subject with preexisting immunity against an immunogen (i.e., an immunogen to be administered to the subject, e.g., an immunogenic delivery vehicle such as, e.g., AAV)).

In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) before the administration of the viral vector(s).

In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) simultaneously with the administration of the viral vector(s).

In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) after the administration of the viral vector(s).

In some embodiments, the viral vectors are administered two or more times and the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) before and/or between each of the administrations of the viral vectors.

In some embodiments, the immunogen re-administration occurs via the same administration route as its prior administration. In some embodiments, the immunogen re-administration occurs via a different administration route than its prior administration. In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) before the administration of the immunogen. In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) simultaneously with the administration of the immunogen. In some embodiments, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) after the administration of the immunogen. In some embodiments, the immunogen is administered two or more times and the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) before and/or between each of the administrations of the immunogen. In some embodiments, the plasma cell depleting agent is administered before the administration of the immunogen. In some embodiments, such as when the immunogen is administered two or more times, the plasma cell depleting agent is administered before and/or between each of the administrations of the immunogen. In some embodiments, the plasma cell depleting agent is administered again within a short period of the first administration. In some embodiments, the plasma cell depleting agent is continuously administered throughout the pre-dose and re-dose periods (e.g., to clear plasma cells and keep plasma cell levels low).

In any of the above methods, the plasma cell depleting agent (optionally in combination with the B cell depleting agent and/or the immunoglobulin depleting agent) can be administered (to a subject with preexisting immunity against an immunogen (i.e., an immunogen to be administered to the subject, e.g., an immunogenic delivery vehicle such as, e.g., AAV)) simultaneously with the immunogen (e.g., nucleic acid construct, nuclease agent or CRISPR/Cas system, e.g., in an immunogenic delivery vehicle) (e.g., an immunogenic delivery vehicle) or not simultaneously (e.g., sequentially in any combination). For example, in a method comprising administering a composition comprising a plasma cell depleting agent (optionally in combination with the B cell depleting agent and/or the immunoglobulin depleting agent) and an immunogen (e.g., nucleic acid construct, nuclease agent or CRISPR/Cas system, e.g., in an immunogenic delivery vehicle), they can be administered separately (e.g., the plasma cell depleting agent (optionally in combination with the B cell depleting agent and/or the immunoglobulin depleting agent) separately from the immunogen (e.g., nucleic acid construct, nuclease agent or CRISPR/Cas system, e.g., in an immunogenic delivery vehicle)). For example, the plasma cell depleting agent (optionally in combination with the B cell depleting agent and/or the immunoglobulin depleting agent) can be administered (to a subject with preexisting immunity against an immunogen (i.e., an immunogen to be administered to the subject, e.g., an immunogenic delivery vehicle such as, e.g., AAV)) prior to the immunogen (e.g., nucleic acid construct, nuclease agent or CRISPR/Cas system, e.g., in an immunogenic delivery vehicle), subsequent to the immunogen (e.g., nucleic acid construct, nuclease agent or CRISPR/Cas system, e.g., in an immunogenic delivery vehicle), prior to and subsequent to the immunogen (e.g., nucleic acid construct, nuclease agent or CRISPR/Cas system, e.g., in an immunogenic delivery vehicle), or at the same time as immunogen (e.g., nucleic acid construct, nuclease agent or CRISPR/Cas system, e.g., in an immunogenic delivery vehicle). In some embodiments, the plasma cell depleting agent is administered (to a subject with preexisting immunity against an immunogen (i.e., an immunogen to be administered to the subject, e.g., an immunogenic delivery vehicle such as, e.g., AAV)) before the administration of the immunogen. In some embodiments, the plasma cell depleting agent is administered again (to a subject with preexisting immunity against an immunogen (i.e., an immunogen to be administered to the subject, e.g., an immunogenic delivery vehicle such as, e.g., AAV)) within a short period of the first administration. In some embodiments, the plasma cell depleting agent is continuously administered (to a subject with preexisting immunity against an immunogen (i.e., an immunogen to be administered to the subject, e.g., an immunogenic delivery vehicle such as, e.g., AAV)) throughout the pre-dose and re-dose periods (e.g., to clear plasma cells and keep plasma cell levels low).

In some embodiments, the plasma cell depleting agent (optionally in combination with the B cell depleting agent and/or the immunoglobulin depleting agent) can be administered (to a subject with preexisting immunity against an immunogen (i.e., an immunogen to be administered to the subject, e.g., an immunogenic delivery vehicle such as, e.g., AAV)) about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the immunogen (e.g., nucleic acid construct, nuclease agent or CRISPR/Cas system, e.g., in an immunogenic delivery vehicle).

In one example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and/or subsequent to administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week prior to and subsequent to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours, about 8 hours, about 12 hours, about 18 hours, about 1 day, about 2 days, about 3 days, about 4 days, about 5 days, about 6 days, or about 1 week after administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent (optionally in combination with the B cell depleting agent and/or the immunoglobulin depleting agent) is administered (to a subject with preexisting immunity against an immunogen (i.e., an immunogen to be administered to the subject, e.g., an immunogenic delivery vehicle such as, e.g., AAV)) within about 6 months after the nucleic acid construct, optionally wherein the nucleic acid construct is in a viral vector, and the plasma cell depleting agent is administered if the viral vector is still present in the subject. In one example, the immunogen (e.g., nucleic acid construct or AAV) is administered within about 3 months, within about 2 months, within about 7 weeks, within about 6 weeks, within about 5 weeks, within about 4 weeks, within about 3 weeks, or within about 2 weeks after an initial dose of the plasma cell depleting agent (optionally in combination with the B cell depleting agent and/or the immunoglobulin depleting agent). In one example, the immunogen (e.g., nucleic acid construct or AAV) is administered at least about 2 weeks, at least about 3 weeks, at least about 4 weeks, at least about 5 weeks, at least about 6 weeks, at least about 7 weeks, at least about 2 months, or at least about 3 months after an initial dose of the plasma cell depleting agent (optionally in combination with the B cell depleting agent and/or the immunoglobulin depleting agent). In one example, the immunogen (e.g., nucleic acid construct or AAV) is administered within about 2 weeks to about 3 months, within about 2 weeks to about 2 months, within about 2 weeks to about 7 weeks, within about 2 weeks to about 6 weeks, within about 2 weeks to about 5 weeks, within about 2 weeks to about 4 weeks, within about 2 weeks to about 3 weeks, within about 2 months to about 3 months, within about 7 weeks to about 3 months, within about 6 weeks to about 3 months, within about 5 weeks to about 3 months, within about 4 weeks to about 3 months, within about 3 weeks to about 3 months, or within about 2 weeks to about 3 months after an initial dose of the plasma cell depleting agent (optionally in combination with the B cell depleting agent and/or the immunoglobulin depleting agent). In one example, the immunogen (e.g., nucleic acid construct or AAV) is administered within about 2 weeks to about 7 weeks, within about 3 weeks to about 6 weeks, within about 4 weeks to about 5 weeks, within about 3 weeks to about 7 weeks, within about 4 weeks to about 7 weeks, within about 5 weeks to about 7 weeks, within about 2 weeks to about 6 weeks, within about 2 weeks to about 5 weeks, within about 2 weeks to about 4 weeks, or within about 4 weeks to about 6 weeks after an initial dose of the plasma cell depleting agent (optionally in combination with the B cell depleting agent and/or the immunoglobulin depleting agent). The timing can depend, for example, on initial starting titer, and which IgG clearing agent is used. In some embodiments, for FcRn blockers, for low titers, the timing may be about 2 days to about 1 week. In some embodiments, for FcRn blockers, for high titers, it the timing may be about 4 to about 7 weeks. In some embodiments, for IgG degrading enzymes, for IdeS, the timing may be about 1 week to about 4 weeks for plasma cell depleting agent, then about 2 days to about 1 week for IdeS.

In some embodiments, e.g., for methods and compositions of the present disclosure involving administration of a plasma cell depleting agent which is a bispecific BCMA×CD3 antibody (e.g., REGN5458) to a subject with preexisting immunity against an immunogen (i.e., an immunogen to be administered to the subject, e.g., an immunogenic delivery vehicle such as, e.g., AAV), the dose of the bispecific BCMA×CD3 antibody (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, the bispecific BCMA×CD3 antibody (e.g., REGN5458) can be administered to the subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the bispecific BCMA×CD3 antibody (e.g., REGN5458) (or pharmaceutical composition thereof) dose is about 20 mg/kg.

In some embodiments, e.g., for methods and compositions of the present disclosure involving administration of a B cell depleting agent which is a bispecific CD20×CD3 antibody (e.g., REGN1979) to a subject with preexisting immunity against an immunogen (i.e., an immunogen to be administered to the subject, e.g., an immunogenic delivery vehicle such as, e.g., AAV) (i.e., in combination with a plasma cell depleting agent), the dose of the bispecific CD20×CD3 antibody (or pharmaceutical compositions thereof) is from about 0.05 mg/kg to about 3 mg/kg, such as from about 0.05 mg/kg to about 0.1 mg/kg, about 0.1 mg/kg to about 0.5 mg/kg, about 0.5 mg/kg to about 1 mg/kg, about 1 mg/kg to about 1.5 mg/kg, about 1.5 mg/kg to about 2 mg/kg, about 2 mg/kg to about 2.5 mg/kg, or about 2.5 mg/kg to about 3 mg/kg. In one specific embodiment, the bispecific CD20×CD3 antibody (e.g., REGN1979) is administered to the subject (i.e., in combination with a plasma cell depleting agent) at a dose of about 0.1 mg/kg. In another specific embodiment, the bispecific CD20×CD3 antibody (e.g., REGN1979) is administered to the subject (i.e., in combination with a plasma cell depleting agent) at a dose of about 1 mg/kg.

In some embodiments, e.g., for methods and compositions of the present disclosure involving administration of an immunoglobulin depleting agent which is efgartigimod to a subject with preexisting immunity against an immunogen (i.e., an immunogen to be administered to the subject, e.g., an immunogenic delivery vehicle such as, e.g., AAV) (i.e., in combination with a plasma cell depleting agent), the dose of efgartigimod (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, efgartigimod can be administered to the subject (i.e., in combination with a plasma cell depleting agent) at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the efgartigimod (or pharmaceutical composition thereof) dose is about 20 mg/kg.

In some embodiments, e.g., for methods and compositions of the present disclosure involving administration of an immunogen which is an AAV to a subject, the dose of the AAV (or pharmaceutical compositions thereof) administered to a subject is between about 1×10⁵ plaque forming units (pfu) to about 1×10¹⁵ pfu. In some cases, the AAV can be administered to the subject at a dose from about 1×10⁸ pfu to about 1×10¹⁵ pfu, or from about 1×10¹⁰ pfu to about 1×10¹⁵ pfu, or from about 1×10⁸ pfu to about 1×10¹² pfu.

In some embodiments, the dose of the AAV (or pharmaceutical compositions thereof) administered to the subject is between about 1×10⁵ vg to about 1×10¹⁶ vg. In certain embodiments, the dose of the AAV administered to the subject is between about 1×10⁶ vg to about 1×10⁹ vg, about 1×10⁷ vg to about 1×10¹⁰ vg, about 1×10⁸ vg to about 1×10¹¹ vg, about 1×10⁹ vg to about 1×10¹² vg, about 1×10¹⁰ vg to about 1×10¹³ vg, about 1×10¹¹ vg to about 1×10¹⁴ vg, about 1×10¹² vg to about 1×10¹⁵ vg, about 1×10¹³ vg to about 1×10¹⁶ vg, or about 1×10¹⁴ vg to about 1×10¹⁶ vg. In certain embodiments, the dose of the AAV administered to the subject is between about 1×10¹⁰ vg to about 1×10¹⁶ vg. In certain embodiments, the dose of the AAV administered to the subject is at least about 1×10⁶ vg, at least about 1×10⁷ vg, at least about 1×10⁸ vg, at least about 1×10⁹ vg, at least about 1×10¹⁰ vg, at least about 1×10¹¹ vg, at least about 1×10¹² vg, at least about 1×10¹² vg, at least about 1×10¹³ vg, at least about 1×10¹⁴ vg, or at least about 1×10¹⁵ vg. In certain embodiments, the vg is total vector genome per subject.

In some embodiments, the dose of the AAV (or pharmaceutical compositions thereof) administered to the subject is about 1×10¹², 1×10¹³, 1×10¹⁴, 1×10¹⁵, and 1×10¹⁶ vector genomes (vg)/mL. Further examples of doses of AAV include about 1×10¹², about 1×10¹³, about 1×10¹⁴, about 1×10¹⁵, and about 1×10¹⁶ vector genomes (vg)/mL, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/mL.

Other examples of doses of AAV (or pharmaceutical compositions thereof) include about 1×10¹², about 1×10¹³, about 1×10¹⁴, about 1×10¹⁵, and about 1×10¹⁶ vector genomes (vg)/kg of body weight, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/kg of body weight. In another example, the viral titer is about 3.33E11 to about 5E13 vg/kg.

In one example, the AAV dose (or pharmaceutical compositions thereof) is between about 1×10¹³ to about 1×10¹⁴ vg/mL or vg/kg. In another example, the AAV dose is between about 1×10¹² to about 1×10¹³ vg/mL or vg/kg (e.g., between about 1×10¹² to about 1×10¹³ vg/kg). In another example, the AAV dose is between about 1×10¹² to about 1×10¹⁴ vg/mL or vg/kg (e.g., between about 1×10¹² to about 1×10¹⁴ vg/kg).

In one specific embodiment, the AAV dose (or pharmaceutical composition thereof) is about 3×10¹¹ vg/kg. In one specific embodiment, the AAV dose (or pharmaceutical composition thereof) is about 6×10¹¹ vg/kg. In another specific embodiment, the AAV dose (or pharmaceutical composition thereof) is about 9×10¹¹ vg/kg. In another specific embodiment, the AAV dose (or pharmaceutical composition thereof) is about 3×10¹² vg/kg. In one specific embodiment, the AAV dose (or pharmaceutical composition thereof) is about 1×10¹³ vg/kg. In another specific embodiment, the AAV dose (or pharmaceutical composition thereof) is about 6×10¹³ vg/kg.

In some embodiments, the dose of the nucleic acid construct (e.g., AAV vector comprising the nucleic acid construct) and the nuclease agent or one or more nucleic acids encoding the nuclease agent (e.g., LNP comprising the nuclease agent or one or more nucleic acids encoding the nuclease agent) achieves therapeutic levels of gene insertion and/or expression, but is low enough that not all target cells (e.g., liver cells or hepatocytes) or target locus (e.g., albumin, or albumin intron 1) sites are modified (e.g. AAV dose between 3.33e11 to 5e13 vg/kg, and LNP dose between 0.3 to 3 mg/kg).

In some embodiments, the dose of the nucleic acid construct (e.g., AAV vector comprising the nucleic acid construct)

### XVI. Kits

The present disclosure further comprises a kit which may comprise any of various compositions or combinations of the present disclosure, including the CD40 inhibitors (e.g., CD40 antigen-binding molecules), nucleic acid constructs, nuclease agents or CRISPR/Cas systems, or pharmaceutical compositions thereof, of the disclosure. Such kits may further comprise plasma cell depleting agents (optionally in combination with B cell depleting agents and/or immunoglobulin depleting agents) or pharmaceutical compositions thereof, of the disclosure.

One exemplary embodiment of the present disclosure comprises a kit comprising (i) a CD40 inhibitor, and (ii) optionally, instructions for use. Another exemplary embodiment of the present disclosure comprises a kit comprising (i) an immunogen (e.g., a nucleic acid construct in an immunogenic delivery vehicle, such as AAV), (ii) a CD40 inhibitor, and (iii) optionally, instructions for use. Optionally, such kits can further comprise a plasma cell depleting agent. Optionally, such kits can further comprise a plasma cell depleting agent and a B cell depleting agent and/or an immunoglobulin depleting agent. For example, the immunogen can be a nucleic acid construct, a polypeptide of interest encoded by the nucleic acid construct, a nuclease agent or one or more nucleic acids encoding the nuclease agent, or a delivery vehicle for the nucleic acid construct, the nuclease agent, or the one or more nucleic acids encoding the nuclease agent. For example, the delivery vehicle can be a recombinant AAV (e.g., AAV comprising a nucleic acid construct described herein).

In one aspect, the present disclosure may include a kit comprising, for example: (a) a container that contains a pharmaceutical composition disclosed herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, the kit may further comprise, for example, without limitation, one or more of (i) a buffer, (ii) a diluent, (iii) a filter, (iv) a needle, and/or (v) a syringe. As a non-limiting example, the container may be a bottle, a vial, a syringe, or test tube. In some embodiments, the container may be a multi-use container. In some the pharmaceutical composition may be lyophilized.

Kits of the present disclosure may comprise a lyophilized formulation of the present disclosure in a suitable container and instructions for its reconstitution and/or use. Suitable containers include, for example, bottles, vials (e.g., dual chamber vials), syringes (such as dual chamber syringes) and test tubes. The container may be formed from a variety of materials such as glass or plastic. The kit and/or container may contain instructions on or associated with the container that indicate directions for reconstitution of the lyophilized formulation and/or use of the kit. For example, the label may indicate that the lyophilized formulation is to be reconstituted to an appropriate peptide concentration. The label may indicate that the formulation is useful or intended for any route of administration disclosed herein, e.g., parenteral administration routes disclosed herein.

The container holding the formulation may be a multi-use vial, which may allow for repeat administrations (e.g., from 2-6 administrations) of the reconstituted formulation. The kit may further comprise a second container comprising a suitable diluent (e.g., sodium bicarbonate solution).

Upon mixing of the diluent and the lyophilized formulation, the final peptide concentration in the reconstituted formulation is reached. The kit may further include other materials desirable from a commercial and/or user standpoint, including, for example, without limitation, other buffers, diluents, filters, needles, syringes, and/or package inserts which may comprise, e.g., instructions for use.

Kits of the present disclosure may have a single container that contains the formulation of the pharmaceutical compositions according to the present disclosure with or without other components (e.g., other compounds or pharmaceutical compositions of these other compounds) or may have a distinct container for each component.

In some embodiments, kits of the disclosure may include a formulation of the disclosure packaged for use in combination with the coadministration of a second compound (such as adjuvants (e.g., GM-CSF, a chemotherapeutic agent, a natural product, a hormone or antagonist, an anti-angiogenesis agent or inhibitor, an apoptosis-inducing agent, or a chelator) or a pharmaceutical composition thereof. The components of the kit may be pre-complexed or each component may be in a separate distinct container prior to administration to a patient. The components of the kit may be provided in one or more liquid solutions. A liquid solution described herein may be an aqueous solution, for example, a sterile aqueous solution. The components of the kit may also be provided as solids, which may be converted into liquids such as by addition of suitable solvents, which may be provided in another distinct container.

The container of a therapeutic kit may be a vial, test tube, flask, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiment, a kit may contain an apparatus (e.g., one or more needles, syringes, eye droppers, pipettes, etc.), which may allow for administration of the agents of the disclosure that are components of the present kit.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 8
Description of Sequences.

| SEQ ID NO | Type | Description |
|---|---|---|
| 1 | DNA | 30027P2 HCVR nucleic acid sequence |
| 2 | Protein | 30027P2 HCVR amino acid sequence |
| 3 | DNA | 30027P2 HCDR1 nucleic acid sequence |
| 4 | Protein | 30027P2 HCDR1 amino acid sequence |
| 5 | DNA | 30027P2 HCDR2 nucleic acid sequence |
| 6 | Protein | 30027P2 HCDR2 amino acid sequence |
| 7 | DNA | 30027P2 HCDR3 nucleic acid sequence |
| 8 | Protein | 30027P2 HCDR3 amino acid sequence |
| 9 | DNA | Common LCVR nucleic acid sequence |
| 10 | Protein | Common LCVR amino acid sequence |
| 11 | DNA | Common LCDR1 nucleic acid sequence |
| 12 | Protein | Common LCDR1 amino acid sequence |
| 13 | DNA | Common LCDR2 nucleic acid sequence |
| 14 | Protein | Common LCDR2 amino acid sequence |
| 15 | DNA | Common LCDR3 nucleic acid sequence |
| 16 | Protein | Common LCDR3 amino acid sequence |
| 17 | DNA | 30027P2 heavy chain nucleic acid sequence |
| 18 | Protein | 30027P2 heavy chain amino acid sequence |
| 19 | DNA | Common light chain nucleic acid sequence |
| 20 | Protein | Common light chain amino acid sequence |
| 21 | DNA | 21519P2 HCVR nucleic acid sequence |
| 22 | Protein | 21519P2 HCVR amino acid sequence |
| 23 | DNA | 21519P2 HCDR1 nucleic acid sequence |
| 24 | Protein | 21519P2 HCDR1 amino acid sequence |
| 25 | DNA | 21519P2 HCDR2 nucleic acid sequence |
| 26 | Protein | 21519P2 HCDR2 amino acid sequence |
| 27 | DNA | 21519P2 HCDR3 nucleic acid sequence |
| 28 | Protein | 21519P2 HCDR3 amino acid sequence |
| 29 | DNA | 21519P2 heavy chain nucleic acid sequence |
| 30 | Protein | 21519P2 heavy chain amino acid sequence |
| 31 | DNA | 21520P2 HCVR nucleic acid sequence |
| 32 | Protein | 21520P2 HCVR amino acid sequence |
| 33 | DNA | 21520P2 HCDR1 nucleic acid sequence |
| 34 | Protein | 21520P2 HCDR1 amino acid sequence |
| 35 | DNA | 21520P2 HCDR2 nucleic acid sequence |
| 36 | Protein | 21520P2 HCDR2 amino acid sequence |
| 37 | DNA | 21520P2 HCDR3 nucleic acid sequence |
| 38 | Protein | 21520P2 HCDR3 amino acid sequence |
| 39 | DNA | 21520P2 heavy chain nucleic acid sequence |
| 40 | Protein | 21520P2 heavy chain amino acid sequence |
| 41 | DNA | REGN16334 D1 heavy chain nucleic acid sequence |
| 42 | Protein | REGN16334 D1 heavy chain amino acid sequence |
| 43 | DNA | REGN16334 and REGN16335 D2 heavy chain nucleic acid sequence |
| 44 | Protein | REGN16334 and REGN16335 D2 heavy chain amino acid sequence |
| 45 | DNA | REGN16335 D1 heavy chain nucleic acid sequence |
| 46 | Protein | REGN16335 D1 heavy chain amino acid sequence |
| 47 | DNA | REGN16431 D1 heavy chain nucleic acid sequence |
| 48 | Protein | REGN16431 D1 heavy chain amino acid sequence |
| 49 | DNA | REGN16431 and REGN16432 D2 heavy chain nucleic acid sequence |
| 50 | Protein | REGN16431 and REGN16432 D2 heavy chain amino acid sequence |
| 51 | DNA | REGN16432 D1 heavy chain nucleic acid sequence |
| 52 | Protein | REGN16432 D1 heavy chain amino acid sequence |
| 53 | Protein | REGN3094 (human CD40 extracellular domain with C-terminal MMH tag; |
| | | “hCD40-MMH”) |
| 54 | Protein | REGN3097 (monkey CD40 extracellular domain with C-terminal MMH tag; |
| | | “mfCD40-MMH”) |
| 55 | Protein | REGN3098 (mouse CD40 extracellular domain with C-terminal MMH tag; |
| | | “mCD40-MMH”) |
| 56 | Protein | REGN3095 (hCD40-mFc) |
| 57 | Protein | Human Factor IX Protein NCBI Accession No. NP_000124.1 |
| 58 | DNA | Human F9 mRNA (cDNA) NCBI Accession No. NM_000133.4 |
| 59 | DNA | Human F9 CDS CCDS ID CCDS14666.1 |
| 60 | DNA | Native CpG removed no splice F9 Insert |
| 61 | DNA | CpG0 F9 Insert |
| 62 | DNA | Insert 30 |
| 63 | Protein | FIX Encoded by F9 Inserts |
| 64 | DNA | Insert 30 no ITRs |
| 65 | RNA | Cas9 mRNA |
| 66 | RNA | Cas9 mRNA CDS |
| 67 | DNA | Cas9 CDS |
| 68 | DNA | Human ALB Intron 1 |
| 69 | DNA | Guide RNA Target Sequence Plus PAM v1 |
| 70 | DNA | Guide RNA Target Sequence Plus PAM v2 |
| 71 | DNA | Guide RNA Target Sequence Plus PAM v3 |
| 72 | Protein | SpCas9 Protein V1 |
| 73 | DNA | SpCas9 DNA V1 |
| 74 | DNA | SpCas9 mRNA (cDNA) |
| 75 | Protein | SpCas9 Protein V2 |
| 76 | RNA | SpCas9 mRNA V2 |
| 77 | Protein | SV40 NLS v1 |
| 78 | Protein | SV40 NLS v2 |
| 79 | Protein | Nucleoplasmin NLS |
| 80 | RNA | crRNA Tail v1 |
| 81 | RNA | crRNA Tail v2 |
| 82 | RNA | TracrRNA v1 |
| 83 | RNA | TracrRNA v2 |
| 84 | RNA | TracrRNA v3 |
| 85 | RNA | gRNA Scaffold v1 |
| 86 | RNA | gRNA Scaffold v2 |
| 87 | RNA | gRNA Scaffold v3 |
| 88 | RNA | gRNA Scaffold v4 |
| 89 | RNA | gRNA Scaffold v5 |
| 90 | RNA | gRNA Scaffold v6 |
| 91 | RNA | gRNA Scaffold v7 |
| 92 | RNA | gRNA Scaffold v8 |
| 93 | RNA | Modified gRNA Scaffold |
| 94-97 | RNA | Human ALB Intron 1 Guide Sequences |
| 98-105 | RNA | Human ALB Intron 1 sgRNA Sequences |
| 106-109 | DNA | Human ALB Intron 1 Guide RNA Target Sequences |
| 110 | DNA | ITR 145 |
| 111 | DNA | ITR 141 |
| 112 | DNA | ITR 130 |
| 113 | DNA | ITR 145 Reverse Complement |
| 114 | Protein | Human GAA Protein (NP_000143.2) |
| 115 | DNA | Human GAA cDNA/mRNA (NM_000152.5) |
| 116 | DNA | Human GAA CDS (CCDS32760.1) |
| 117 | Protein | Human GAA (70-952) Protein |
| 118 | DNA | Human GAA (70-952) CDS |
| 119 | Protein | 12450 anti-CD63 scFv |
| 120 | Protein | 12450 anti-CD63 scFv:GAA (70-952) Fusion Protein |
| 121 | Protein | Anti-hTfR scFvs |
| 122 | DNA | 12847 GS 0v2 anti-TfR scFv |
| 123 | DNA | ITR 141 Reverse Complement |
| 124 | DNA | ITR 130 Reverse Complement |
| 125 | DNA | 2 × Fix 12847 anti-TfR:GAA Coding Sequence |
| 126 | Protein | 12847 anti-TfR:GAA Fusion Protein |
| 127 | DNA | GAA (70-952) Coding Sequence [1830_GGTGGT_>_GGGGGC] |
| | | [3078_GT_>_GG] |
| 128 | DNA | 6 × Fix anti-CD63:GAA Coding Sequence |
| 129 | DNA | 6 × Fix anti-CD63 scFv Coding Sequence |
| 130 | DNA | VVT1125/VVT1261 - pAAV-12847GAA(GS0v2)-2Xfix-bGH.sv40LuniPA |
| 131 | DNA | VVT1127 - pAAV-CD63GAA-SV40E-MAZ |
| 132 | DNA | VVT1128/VVT1263 - pAAV-CD63GAA-6Xfix-bGH.sv40LuniPA |
| 133 | DNA | VVT1125VVT/1261 - pAAV-12847GAA(GS0v2)-2Xfix-bGH.sv40LuniPA - no |
| | | ITRs |
| 134 | DNA | VVT1128/VVT1263 - pAAV-CD63GAA-6Xfix-bGH.sv40LuniPA - no ITRs |
| 135-281 | Protein | CD40 and CD40L Binding Protein Sequences |
| 282 | DNA | REGN20484 D1 Heavy Chain Nucleic Acid Sequence |
| 283 | Protein | REGN20484 D1 Heavy Chain Amino Acid Sequence |
| 284 | DNA | REGN20484 D2 Heavy Chain Nucleic Acid Sequence |
| 285 | Protein | REGN20484 D2 Heavy Chain Amino Acid Sequence |
| 286 | DNA | Anti-BCMA HCVR DNA Sequence |
| 287 | Protein | Anti-BCMA HCVR Protein Sequence |
| 288 | DNA | Anti-BCMA HCDR1 DNA Sequence |
| 289 | Protein | Anti-BCMA HCDR1 Protein Sequence |
| 290 | DNA | Anti-BCMA HCDR2 DNA Sequence |
| 291 | Protein | Anti-BCMA HCDR2 Protein Sequence |
| 292 | DNA | Anti-BCMA HCDR3 DNA Sequence |
| 293 | Protein | Anti-BCMA HCDR3 Protein Sequence |
| 294 | DNA | Anti-BCMA LCVR DNA Sequence |
| 295 | Protein | Anti-BCMA LCVR Protein Sequence |
| 296 | DNA | Anti-BCMA LCDR1 DNA Sequence |
| 297 | Protein | Anti-BCMA LCDR1 Protein Sequence |
| 298 | DNA | Anti-BCMA LCDR2 DNA Sequence |
| 299 | Protein | Anti-BCMA LCDR2 Protein Sequence |
| 300 | DNA | Anti-BCMA LCDR3 DNA Sequence |
| 301 | Protein | Anti-BCMA LCDR3 Protein Sequence |
| 302 | DNA | Common LCVR DNA Sequence |
| 303 | Protein | Common LCVR Protein Sequence |
| 304 | DNA | Common LCDR1 DNA Sequence |
| 305 | Protein | Common LCDR1 Protein Sequence |
| 306 | DNA | Common LCDR2 DNA Sequence |
| 307 | Protein | Common LCDR2 Protein Sequence |
| 308 | DNA | Common LCDR3 DNA Sequence |
| 309 | Protein | Common LCDR3 Protein Sequence |
| 310 | DNA | Anti-CD3 HCVR DNA Sequence - REGN5458 |
| 311 | Protein | Anti-CD3 HCVR Protein Sequence - REGN5458 |
| 312 | DNA | Anti-CD3 HCDR1 DNA Sequence - REGN5458 |
| 313 | Protein | Anti-CD3 HCDR1 Protein Sequence - REGN5458 |
| 314 | DNA | Anti-CD3 HCDR2 DNA Sequence - REGN5458 |
| 315 | Protein | Anti-CD3 HCDR2 Protein Sequence - REGN5458 |
| 316 | DNA | Anti-CD3 HCDR3 DNA Sequence - REGN5458 |
| 317 | Protein | Anti-CD3 HCDR3 Protein Sequence - REGN5458 |
| 318 | DNA | Anti-CD3 HCVR DNA Sequence - REGN5459 |
| 319 | Protein | Anti-CD3 HCVR Protein Sequence - REGN5459 |
| 320 | DNA | Anti-CD3 HCDR1 DNA Sequence - REGN5459 |
| 321 | Protein | Anti-CD3 HCDR1 Protein Sequence - REGN5459 |
| 322 | DNA | Anti-CD3 HCDR2 DNA Sequence - REGN5459 |
| 323 | Protein | Anti-CD3 HCDR2 Protein Sequence - REGN5459 |
| 324 | DNA | Anti-CD3 HCDR3 DNA Sequence - REGN5459 |
| 325 | Protein | Anti-CD3 HCDR3 Protein Sequence - REGN5459 |
| 326 | Protein | Anti-BCMA Heavy Chain Protein Sequence (IgG4 Heavy Chain Constant Region) |
| 327 | Protein | Anti-CD3 Heavy Chain Protein Sequence (IgG4 Heavy Chain Constant Region with |
| | | H435R/Y436F) |
| 328 | Protein | Common Anti-BCMA and Anti-CD3 Light Chain Protein Sequence (Kappa Light |
| | | Chain Constant Region) |
| 329 | Protein | Anti-CD20 HCVR Protein Sequence |
| 330 | Protein | Common LCVR Protein Sequence |
| 331 | Protein | Anti-CD3 HCVR Protein Sequence |
| 332 | Protein | Anti-CD20 HCDR1 Protein Sequence |
| 333 | Protein | Anti-CD20 HCDR2 Protein Sequence |
| 334 | Protein | Anti-CD20 HCDR3 Protein Sequence |
| 335 | Protein | Common LCDR1 Protein Sequence |
| 336 | Protein | Common LCDR2 Protein Sequence |
| 337 | Protein | Common LCDR3 Protein Sequence |
| 338 | Protein | Anti-CD3 HCDR1 Protein Sequence |
| 339 | Protein | Anti-CD3 HCDR2 Protein Sequence |
| 340 | Protein | Anti-CD3 HCDR3 Protein Sequence |
| 341 | Protein | CD40-CD40L blocking cyclic heptapetide |
| 342 | Protein | CH2 Hinge Sequence |

### EXAMPLES

### Example 1. Construction of Anti-CD40×CD40 Bispecific Antibodies

### Generation of Parental Anti-CD40 Antibodies

Antibodies against CD40 were obtained by immunizing a VELOCIMMUNE® mouse (i.e., an engineered mouse comprising DNA encoding human Immunoglobulin heavy and kappa chain variable regions) with a human CD40 antigen (human CD40 extracellular domain with C-terminal MMH tag; SEQ ID NO: 53).

Following immunization, antibodies were isolated directly from antigen-positive mouse B cells, e.g., as described in U.S. Pat. No. 7,582,298, incorporated by reference herein. Using this method, fully human anti-CD40 antibodies (i.e., antibodies possessing human variable domains and human constant domains) were obtained. Antibodies generated using this method were characterized and selected for desirable characteristics, including affinity, selectivity, etc.

Anti-CD40 antibodies generated using this method include antibodies designated 30027P2, 21519P2, and 21520P2. Certain biological properties of the exemplary CD40 antibodies generated in accordance with the methods of this Example are described in detail in the Examples set forth below.

TABLE 9
Amino Acid Sequence Identifiers for Parental Anti-CD40 Monoclonal Antibodies.

| Antibody | SEQ ID NOS: | | | | | | | | | |
|---|---|---|---|---|---|---|---|---|---|---|
| Designation | HCVR | HCDR1 | HCDR2 | HCDR3 | HC | LCVR | LCDR1 | LCDR2 | LCDR3 | LC |
| 30027P2 | 2 | 4 | 6 | 8 | 18 | 10 | 12 | 14 | 16 | 20 |
| 21519P2 | 22 | 24 | 26 | 28 | 30 | 10 | 12 | 14 | 16 | 20 |
| 21520P2 | 32 | 34 | 36 | 38 | 40 | 10 | 12 | 14 | 16 | 20 |

TABLE 10
Nucleic Acid Sequence Identifiers for Parental Anti-CD40 Monoclonal Antibodies.

| Antibody | | SEQ ID NOS: | | | | | | | | |
|---|---|---|---|---|---|---|---|---|---|---|
| Designation | HCVR | HCDR1 | HCDR2 | HCDR3 | HC | LCVR | LCDR1 | LCDR2 | LCDR3 | LC |
| 30027P2 | 1 | 3 | 5 | 7 | 17 | 9 | 11 | 13 | 15 | 19 |
| 21519P2 | 21 | 23 | 25 | 27 | 29 | 9 | 11 | 13 | 15 | 19 |
| 21520P2 | 31 | 33 | 35 | 37 | 39 | 9 | 11 | 13 | 15 | 19 |

### Generation of Anti-CD40×CD40 Bispecific Antibodies

Bispecific antibodies comprising a first CD40 binding arm (“first antigen-binding domain”) and a second CD40 binding arm (“second antigen-binding domain”) were constructed using standard methodologies, wherein the two CD40 binding arms comprise distinct HCVRs paired with a common light chain. Two different heavy chain constant regions, e.g., as described in U.S. Pat. No. 11,518,807, were used for each CD40 binding arm. Exemplary anti-CD40×CD40 bispecific antibodies were generated in accordance with the present Example and comprise the sequences shown below in Tables 11-12.

As shown in Table 11, for REGN16334 and REGN16431, the first CD40-binding arm (“D1”) comprises the HCVR sequences of parental antibody 21519P2 and the second CD40-binding arm (“D2”) comprises the HCVR sequences of parental antibody 21520P2; REGN16334 and REGN16431 have different constant region modifications for reducing Fc receptor binding and effector function.

For REGN16335 and REGN16432, the first CD40-binding arm (“D1”) comprises the HCVR sequences of parental antibody 30027P2 and the second CD40-binding arm (“D2”) comprises the HCVR sequences of parental antibody 21520P2; REGN16335 and REGN16432 have different constant region modifications for reducing Fc receptor binding and effector function.

For REGN20484, the first CD40-binding arm (“D1”) comprises the HCVR sequences of parental antibody 21520P2 and the second CD40-binding arm (“D2”) comprises the HCVR sequences of parental antibody 30027P2.

REGN16334, REGN16335, REGN16431, REGN16432, and REGN20484 all comprise a common light chain sequence.

TABLE 11
Amino Acid Sequence Identifiers for Anti-CD40 × CD40 Bispecific Antibodies.

| | Anti-CD40 | | | | | Anti-CD40 | | | | | | | | | |
|---|---|---|---|---|---|---|---|---|---|---|---|---|---|---|---|
| | First Antigen-Binding | | | | | Second Antigen-Binding | | | | | | | | | |
| Bispecific | Domain (D1) | | | | | Domain (D2) | | | | | Common Light Chain | | | | |
| Antibody | D1- | D1- | D1- | D1- | D1 | D2- | D2- | D2- | D2- | D2 | Variable Region | | | | |
| Identifier | HCVR | HCDR1 | HCDR2 | HCDR3 | HC | HCVR | HCDR1 | HCDR2 | HCDR3 | HC | LCVR | LCDR1 | LCDR2 | LCDR3 | LC |
| REGN16334 | 22 | 24 | 26 | 28 | 42 | 32 | 34 | 36 | 38 | 44 | 10 | 12 | 14 | 16 | 20 |
| REGN16335 | 2 | 4 | 6 | 8 | 46 | 32 | 34 | 36 | 38 | 44 | 10 | 12 | 14 | 16 | 20 |
| REGN16431 | 22 | 24 | 26 | 28 | 48 | 32 | 34 | 36 | 38 | 50 | 10 | 12 | 14 | 16 | 20 |
| REGN16432 | 2 | 4 | 6 | 8 | 52 | 32 | 34 | 36 | 38 | 50 | 10 | 12 | 14 | 16 | 20 |
| REGN20484 | 32 | 34 | 36 | 38 | 283 | 2 | 4 | 6 | 8 | 285 | 10 | 12 | 14 | 16 | 20 |

TABLE 12
Nucleic Acid Sequence Identifiers for Anti-CD40 × CD40 Bispecific Antibodies.

| | Anti-CD40 | | | | | Anti-CD40 | | | | | | | | | |
|---|---|---|---|---|---|---|---|---|---|---|---|---|---|---|---|
| | First Antigen-Binding | | | | | Second Antigen-Binding | | | | | | | | | |
| Bispecific | Domain (D1) | | | | | Domain (D2) | | | | | Common Light Chain | | | | |
| Antibody | D1- | D1- | D1- | D1- | D1 | D2- | D2- | D2- | D2- | D2 | Variable Region | | | | |
| Identifier | HCVR | HCDR1 | HCDR2 | HCDR3 | HC | HCVR | HCDR1 | HCDR2 | HCDR3 | HC | LCVR | LCDR1 | LCDR2 | LCDR3 | LC |
| REGN16334 | 21 | 23 | 25 | 27 | 41 | 31 | 33 | 35 | 37 | 43 | 9 | 11 | 13 | 15 | 19 |
| REGN16335 | 1 | 3 | 5 | 7 | 45 | 31 | 33 | 35 | 37 | 43 | 9 | 11 | 13 | 15 | 19 |
| REGN16431 | 21 | 23 | 25 | 27 | 47 | 31 | 33 | 35 | 37 | 49 | 9 | 11 | 13 | 15 | 19 |
| REGN16432 | 1 | 3 | 5 | 7 | 51 | 31 | 33 | 35 | 37 | 49 | 9 | 11 | 13 | 15 | 19 |
| REGN20484 | 31 | 33 | 35 | 37 | 282 | 1 | 3 | 5 | 7 | 284 | 9 | 11 | 13 | 15 | 19 |

### Example 2. Biacore Binding Kinetics of CD40 Bivalent Parental and Anti-CD40×CD40 Bispecific Antibodies

The equilibrium dissociation constants (K_{D}) for anti-CD40 bivalent and bispecific monoclonal antibodies (mAbs) were determined using a real-time surface plasmon resonance (SPR)-based Biacore 4000 biosensor. All binding studies were performed in 10 mM HEPES, 150 mM NaCl, 3 mM EDTA, and 0.05% v/v surfactant Tween-20, pH 7.4 (HBS-ET) running buffer at 25° C. and 37° C. The Biacore CM5 sensor surface was first derivatized by amine coupling with a monoclonal mouse anti-human Fc antibody (REGN2567) to capture anti-CD40 bivalent parental and anti-CD40×CD40 bispecific antibodies. Different concentrations of CD40 reagents, human CD40 extracellular domain expressed with a C-terminal myc-myc-hexahistidine tag (“hCD40-MMH”; REGN3094; SEQ ID NO: 53), monkey CD40 extracellular domain expressed with a C-terminal myc-myc-hexahistidine tag (“mfCD40-MMH”; REGN3097; SEQ ID NO: 54), and mouse CD40 extracellular domain expressed with a C-terminal myc-myc-hexahistidine tag (“mCD40-MMH”; REGN3098; SEQ ID NO: 55), at concentrations ranging from 3.7 nM to 100 nM or 3.3 nM to 90 nM in a series of 3-fold dilutions prepared in HBS-ET running buffer were injected at a flow rate of 30 L/min for 4 minutes or 50 L/min for 5 minutes. The dissociation of different CD40 reagents bound to anti-CD40 bivalent parental and anti-CD40×CD40 bispecific antibodies was monitored for 10 minutes in HBS-ET running buffer. At the end of each cycle, the anti-CD40 bivalent parental and anti-CD40×CD40 bispecific antibodies capture surface was regenerated using a 12 sec injection of 20 mM H₃PO₄.

The association rate (ka) and dissociation rate (kd) were determined by fitting the real-time binding sensorgrams to a 1:1 binding model with mass transport limitation using Scrubber 2.0c curve-fitting software. Binding dissociation equilibrium constant (K_{D}) and dissociative half-life (t½) were calculated from the kinetic rates as: K_{D} (M)=k_{d}/kₐ, and t_{1/2} (min)=[ln(2)/(60*k_{d})].

Binding kinetics parameters for different CD40 reagents to anti-CD40 bivalent parental and anti-CD40×CD40 bispecific antibodies at 25° C. and 37° C. are shown in Tables 13-18.

As shown in Table 13, at 25° C., anti-CD40 bivalent parental and anti-CD40×CD40 bispecific antibodies bound to hCD40-MMH with K_{D} values ranging from 101 pM to 25.1 nM. At 25° C., anti-CD40 bivalent parental antibodies bound to mfCD40-MMH with K_{D} values ranging from 901 pM to 205 nM (Table 14). Anti-CD40 bivalent parental antibodies did not bind to mCD40-MMH at 25° C. (Table 15).

As shown in Table 16, at 37° C., anti-CD40 bivalent parental and anti-CD40×CD40 bispecific antibodies bound to hCD40-MMH with KD values ranging from 101 pM to 69.9 nM. At 37° C., anti-CD40 bivalent parental antibodies bound to mfCD40-MMH with KD value 3.16 nM (Table 17). Anti-CD40 bivalent parental antibodies did not bind to mCD40-MMH at 37° C. (Table 18).

TABLE 13
Kinetic binding parameters for the interaction of hCD40-MMH with anti-CD40
bivalent parental and anti-CD40 × CD40 bispecific antibodies at 25° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 428 ± 0.7 | 99.0 | 3.85E+05 | 3.39E−03 | 8.82E−09 | 3.4 |
| (parental) | | | | | | |
| 21520P2 | 416 ± 1 | 76.6 | 8.85E+05 | 2.23E−02 | 2.51E−08 | 0.5 |
| (parental) | | | | | | |
| 30027P2 | 233.0 ± 0.5 | 62.0 | 5.46E+05 | 9.98E−04 | 1.83E−09 | 11.6 |
| (parental) | | | | | | |
| REGN16431 | 348.6 ± 23.1 | 61.2 | 1.47E+06 | 1.49E−04 | 1.01E−10 | 77.5 |
| (bispecific) | | | | | | |
| REGN16432 | 372.4 ± 19.1 | 56.6 | 7.69E+05 | 1.25E−04 | 1.63E−10 | 92.3 |
| (bispecific) | | | | | | |

TABLE 14
Kinetic binding parameters for the interaction of mfCD40-MMH with anti-CD40
bivalent parental and anti-CD40 × CD40 bispecific antibodies at 25° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 428 ± 0.4 | 10.3 | 8.86E+05 | 1.81E−01 | 2.05E−07 | 0.1 |
| (parental) | | | | | | |
| 21520P2 | 417 ± 0.4 | 10.1 | 3.00E+06 | 2.03E−01 | 6.77E−08 | 0.1 |
| (parental) | | | | | | |
| 30027P2 | 232.8 ± 0.5 | 63.1 | 1.14E+06 | 1.02E−03 | 9.01E−10 | 11.3 |
| (parental) | | | | | | |
| REGN16431 | NT | NT | NT | NT | NT | NT |
| (bispecific) | | | | | | |
| REGN16432 | NT | NT | NT | NT | NT | NT |
| (bispecific) | | | | | | |

NT = not tested

TABLE 15
Kinetic binding parameters for the interaction of mCD40-MMH with anti-CD40
bivalent parental and anti-CD40 × CD40 bispecific antibodies at 25° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 426 ± 0 | 0.4 | NB | NB | NB | NB |
| (parental) | | | | | | |
| 21520P2 | 417 ± 0.6 | −0.5 | NB | NB | NB | NB |
| (parental) | | | | | | |
| 30027P2 | 231.5 ± 0.4 | 0.2 | NB | NB | NB | NB |
| (parental) | | | | | | |
| REGN16431 | NT | NT | NT | NT | NT | NT |
| (bispecific) | | | | | | |
| REGN16432 | NT | NT | NT | NT | NT | NT |
| (bispecific) | | | | | | |

NB = no binding was observed under the experimental conditions;
NT = not tested

TABLE 16
Kinetic binding parameters for the interaction of hCD40-MMH with anti-CD40
bivalent parental and anti-CD40 × CD40 bispecific antibodies at 37° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 584 ± 1.7 | 104.6 | 6.35E+05 | 1.23E−02 | 1.94E−08 | 0.9 |
| (parental) | | | | | | |
| 21520P2 | 553 ± 1.1 | 67.2 | 1.15E+06 | 8.03E−02 | 6.99E−08 | 0.1 |
| (parental) | | | | | | |
| 30027P2 | 265.8 ± 0.6 | 66.5 | 6.20E+05 | 3.72E−03 | 6.00E−09 | 3.1 |
| (parental) | | | | | | |
| REGN16431 | 348.6 ± 23.1 | 61.2 | 1.47E+06 | 1.49E−04 | 1.01E−10 | 77.5 |
| (bispecific) | | | | | | |
| REGN16432 | 372.4 ± 19.1 | 56.6 | 7.69E+05 | 1.25E−04 | 1.63E−10 | 92.3 |
| (bispecific) | | | | | | |

TABLE 17
Kinetic binding parameters for the interaction of mfCD40-MMH with anti-CD40
bivalent parental and anti-CD40 × CD40 bispecific antibodies at 37° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 583 ± 0.7 | 8.4 | IC | IC | IC | IC |
| (parental) | | | | | | |
| 21520P2 | 551 ± 0.5 | 7.7 | IC | IC | IC | IC |
| (parental) | | | | | | |
| 30027P2 | 263.3 ± 1.1 | 68.5 | 1.05E+06 | 3.33E−03 | 3.16E−09 | 3.5 |
| (parental) | | | | | | |
| REGN16431 | NT | NT | NT | NT | NT | NT |
| (bispecific) | | | | | | |
| REGN16432 | NT | NT | NT | NT | NT | NT |
| (bispecific) | | | | | | |

IC = inconclusive;
NT = not tested

TABLE 18
Kinetic binding parameters for the interaction of mCD40-MMH with anti-CD40
bivalent parental and anti-CD40 × CD40 bispecific antibodies at 37° C.

| | mAb Capture | 100 nM Ag | kₐ | k_{d} | K_{D} | t½ |
|---|---|---|---|---|---|---|
| Antibody | Level (RU) | Bound (RU) | (1/Ms) | (1/s) | (M) | (min) |
| 21519P2 | 580 ± 0.1 | 1.6 | NB | NB | NB | NB |
| (parental) | | | | | | |
| 21520P2 | 548 ± 0.4 | 0.2 | NB | NB | NB | NB |
| (parental) | | | | | | |
| 30027P2 | 260.4 ± 1.1 | 0.2 | NB | NB | NB | NB |
| (parental) | | | | | | |
| REGN16431 | NT | NT | NT | NT | NT | NT |
| (bispecific) | | | | | | |
| REGN16432 | NT | NT | NT | NT | NT | NT |
| (bispecific) | | | | | | |

NB = no binding was observed under the experimental conditions;
NT = not tested

### Example 3. Cross-Competition Between Different Anti-CD40 Monoclonal Antibodies

Binding competition between different anti-CD40 monoclonal antibodies (mAbs) was determined using a real time, label-free bio-layer interferometry (BLI) assay on the Octet HTX biosensor platform (Pall ForteBio Corp.). In addition to parental antibodies 21519P2, 21520P2, and 30027P2, a comparator anti-CD40 antibody (REGN11209) was also tested; this comparator has the heavy chain and light chain sequences of iscalimab (see, U.S. Pat. No. 8,828,396). The entire experiment was performed at 25° C. in 10 mM HEPES buffer containing 150 mM NaCl, 3 mM EDTA, 1 mg/mL BSA, 0.02% NaN₃, and 0.05% v/v Surfactant Tween-20 at pH 7.4 (HBS-EP) with the plate shaking at a speed of 1000 rpm.

To assess the ability of one antibody to compete with another antibody for binding to CD40, around 0.47 nm-0.54 nm of recombinant human CD40 extracellular domain expressed with a C-terminal myc-myc-hexahistidine (hCD40-MMH; SEQ ID NO: 53) was first captured onto anti-Penta-His antibody coated Octet biosensor tips (Fortebio Inc, #18-5122) by submerging the biosensor tips in wells containing 10 μg/mL solution of the hCD40-MMH for 90 seconds. The antigen captured biosensor tips were then saturated with a first anti-CD40 monoclonal antibody (subsequently referred to as “mAb-1”) by dipping into wells containing 50 μg/mL solution of mAb-1 for 4 minutes. The biosensor tips were then subsequently dipped into wells containing 50 μg/mL solution of a second anti-CD40 monoclonal antibody (subsequently referred to as “mAb-2”) for 3 minutes. The biosensor tips were washed in HBS-EBT buffer in between every step of the experiment. The real-time binding response was monitored and the binding response at the end of every step was recorded. The response of mAb-2 binding to hCD40-MMH pre-complexed with mAb-1 was compared to the binding response hCD40-MMH alone (sample of isotype control), and if pre-bound mAb-1 reduced binding of mAb-2 by more than 50%, mAb-1 was considered to be a competitor to mAb-2.

Competitors of each antibody tested are summarized in Table 19 below. Parental antibodies 21519P2 and 21520P2 were found to compete with each other, but not with 30027P2, for binding to hCD40-MMH.

TABLE 19
Cross-competition between different anti-CD40
monoclonal antibodies for binding to hCD40-MMH.

| Prebound mAb-1 | mAb-2 blocked by mAb-1 (>50%) |
|---|---|
| 21519P2 | 21519P2 |
| | 21520P2 |
| 21520P2 | 21520P2 |
| | 21519P2 |
| 30027P2 | 30027P2 |
| REGN11209 | REGN11209 |
| | 21519P2 |
| | 21520P2 |

### Example 4. ELISA Assay to Assess Blocking Activity of Anti-CD40×CD40 Bispecific Antibodies

An ELISA-based blocking assay was developed to determine the ability of C40×CD40 bispecific antibodies to block the binding of the hCD40 monomer to plate-coated hCD40L. The recombinant human CD40-mmH protein (hCD40-MMH; REGN3094; SEQ ID NO:53) used in the experiments comprises a portion of the human CD40 extracellular domain (amino acids P20-R193) fused to 2×Myc peptide and 6×histadine at the C-terminus of human CD40, and the human CD40L (accession #NM_000074.2) with amino acids E108-L261 of the extracellular domain with 9× His-2×(SGGG)-IGER at the N-terminus (9His-hCD40L) was commercially obtained from Biolegend.

In the blocking assay, 9His-hCD40L was passively absorbed at a concentration of 5 μg/mL in PBS on a 96-well microtiter plate overnight at 4° C. Nonspecific binding sites were subsequently blocked using a 0.5% (w/v) solution of BSA in PBS. In a separate 96-well microtiter plate, a fixed amount of 40 nM hCD40-mmH was pre-mixed with one of the following antibodies, at concentrations ranging from 977 pM to 1 μM in PBS+0.5% BSA: (1) anti-CD40×CD40 bispecific antibodies (REGN16431, REGN16432, REGN16634, and REGN16335); (2) parental anti-CD40 mAbs (bivalent antibodies comprising 2 fragment antigen-binding [Fab] arms identical to one of anti-CD40 Fab of anti-CD40×CD40 antibodies); (3) anti-CD40×Irrelevant antibodies (bivalent antibodies that incorporate one Fab arm identical to one of anti-CD40 Fab of anti-CD40×CD40 antibodies and another Fab arm specific to an irrelevant antigen); and (4) human IgG4 with Fc mutation isotype control antibodies (REGN7540 and REGN4513). The fixed concentration of hCD40-mmH was selected to be near the concentration (EC₅₀ value) that generated 50% of the maximal binding to the plate-adhered 9His-hCD40L. After one-hour incubation, the antibody-antigen complexes were transferred to the microtiter plate coated with 9His-hCD40L. After one hour incubation at room temperature, the plates were washed, and plate-bound hCD40-mmH protein was detected with horseradish peroxidase (HRP) conjugated goat anti-c-Myc antibody. The plates were then developed using TMB substrate solution (BD Biosciences) according to the manufacturer's recommended procedure and the absorbance at 450 nm (OD₄₅₀) was measured on a SpectraMax i3× plate reader.

Binding data were analyzed using a sigmoidal (four-parameter logistic) dose-response model with GraphPad Prism™ software. The IC₅₀ value, defined as the concentration of antibody required to block 50% of 40 nM hCD40-mmH binding to plate-coated 9His-hCD40L, was determined to indicate blocking potency. The percent blocking of tested antibodies at the highest tested concentration (1 pM) was calculated based on the formula shown below:

$$\% Blocking = 100 - ( \frac{[ Experimental {Signal}_{( 300 nM Ab )} - Background {Signal}_{( buffer )} ]}{[ Maximum {Signal}_{( lnM hCD 40 - mFc alone )} - Background {Signal}_{( buffer )} ]} ) \times 100$$

where the maximum signal was the interpolated binding signal for 40 nM hCD40-mmH from the hCD40-mmH concentration-response curve. Antibodies that blocked binding of hCD40 greater than 50% were classified as blockers. Antibodies that blocked binding equal or less than 50% were classified as non-blockers. IC₅₀ values for nonblockers were not determined

### Results

The ability of anti-CD40×CD40 bispecific antibodies to block human CD40 monomer binding to plate-coated human CD40L was assessed using a sandwich ELISA-based blocking assay. The results are shown in Table 20. As shown in Table 20, each of the CD40×CD40 bispecific antibodies REGN16431, REGN16432, REGN16334, and REGN16335 displayed concentration-dependent blocking of hCD40 binding to hCD40L with 93% to 98% blocking at the highest antibody concentration tested (1 pM). The IC₅₀ values for these bispecific antibodies are similar around 32 nM. The four parental anti-CD40 antibodies (H4sH21519P2, H4sH21520P2, REGN17288, REGN17544) displayed maximum blocking ranging from 91% to 94% and IC₅₀ values from 52 nM to 71 nM. Four anti-CD40×Irrelevant antibodies (REGN17551, REGN17552, REGN17548, RENG17549) also inhibited hCD40 binding to hCD40L with blocking ranging from 83% to 87%. Two parental anti-CD40 antibodies (H4sH30027P2 and REGN17289) and two anti-CD40×Irrelevant antibodies (REGN17553 and REGN17550) showed minimum blocking activity around 10% and were classified as non-blockers. In this experiment the two human IgG4 with Fc mutation isotype control antibodies (REGN7540, REGN4513) showed no blocking activity, as expected.

TABLE 20
Summary of anti-CD40 antibodies blocking human monomer dimer binding to human CD40L.

| | | mAb blocking 40nM hCD40-mmH | |
|---|---|---|---|
| | | binding to 9His-hCD40L | |
| Antibody ID | Antibody format | IC₅₀ (M) | % Blocking at 1 M mAb |
| REGN16431 | CD40 × CD40 | 3.3E−08 | 93 |
| REGN16432 | CD40 × CD40 | 3.2E−08 | 95 |
| REGN16334 | CD40 × CD40 | 3.2E−08 | 98 |
| REGN16335 | CD40 × CD40 | 3.2E−08 | 96 |
| H4sH21519P2 | Parental CD40 | 6.1E−08 | 94 |
| H4sH21520P2 | Parental CD40 | 6.2E−08 | 94 |
| H4sH30027P2 | Parental CD40 | NBL | 10 |
| REGN17288 | Parental CD40 | 5.2E−08 | 94 |
| REGN17544 | Parental CD40 | 7.1E−08 | 91 |
| REGN17289 | Parental CD40 | NBL | 10 |
| REGN17551 | CD40 × Irrelevant | ND | 87 |
| REGN17552 | CD40 × Irrelevant | ND | 84 |
| REGN17553 | CD40 × Irrelevant | NBL | 10 |
| REGN17548 | CD40 × Irrelevant | ND | 86 |
| REGN17549 | CD40 × Irrelevant | ND | 83 |
| REGN17550 | CD40 × Irrelevant | NBL | 10 |
| REGN7540 | Human IgG4 with Fc mutation isotype control 1 | NBL | −6 |
| REGN4513 | Human IgG4 with Fc mutation isotype control 2 | NBL | 0 |

NBL: Non-blocking (% blocking is less than or equal to 50%).
ND: Not determined (no sigmoidal curve fit observed to calculate IC50 values).

### Example 5. Anti-CD40×CD40 Bispecific Antibody Binding to Cell-Surface CD40 as Measured by Flow Cytometry

Flow cytometry was used to assess the ability of anti-CD40×CD40 bispecific antibodies to bind human CD40 (hCD40) or Macaca fascicularis CD40 (mfCD40) expressing cells. Human embryonic kidney 293 (HEK293) cells stably expressing luciferase reporter gene under the control of the nuclear factor kappa-light-chain-enhancer of activated B cells (NF-kB) and enhanced green fluorescent protein (HEK293/D9) were engineered to express hCD40 (accession #P25942-1) or mfCD40 (accession #XP_005569274.1) by transfecting the cells with neomycin resistant pRG984 plasmid encoding full-length hCD40 (amino acids M1-Q277, HEK293/D9/hCD40), or neomycin resistant pRG984 plasmid encoding full-length mfCD40 (amino acids M1-Q282, HEK293/D9/mfCD40). Ramos 2G6.4C10 cells were utilized to evaluate the binding of anti-CD40 antibodies to hCD40 endogenously expressed on the cell surface. CD40 negative HEK293/D9 cells that showed no detectable expression of CD40 by flow cytometry with a commercial anti-CD40 antibody were included as a background binding control.

Experiments were carried out according to the following procedure: HEK293/D9/hCD40, HEK293/D9/mfCD40 or HEK293/D9 cells were rinsed once in 1×PBS buffer without Ca²⁺/Mg²⁺ and incubated for 10 minutes at 37° C. with Enzyme Free Cell Dissociation Solution to detach cells from flask. The dissociated cells or Ramos 2G6.4G10 suspension cells were washed with 1×PBS and counted with Cellometer™ Auto T4 cell counter (Nexcelom Bioscience, Lawrence, MA). Cells were then resuspended to 1×10⁷ per mL in 1×PBS and separately stained with 2.5 pM of CellTrace™ reagents (Invitrogen, Carlsbad, CA) for 20 minutes at room temperature (RT) to generate a unique fluorescence signature for each cell line (CellTrace™ CFSE for Ramos 2G6.4C10 cells, CellTrace™ Violet for HEK293/D9/mfCD40 cells, CellTrace™ Yellow for HEK293/D9 cells, and HEK293/D9/hCD40 cells were unstained). CellTrace™ labeling reaction was stopped by adding FBS to a final concentration of 25% in 1×PBS followed by a 5-minute incubation at room temperature to quench unbound dye in solution. Cells were washed with 1×PBS and equal numbers of each of the four cell lines, stained and unstained, were mixed in a ratio of 1:1:1:1 for multiplexing. Approximately 2×10⁵ cells per well were seeded onto 96-well Corning plates and stained with LIVE/DEAD™ Fixable Near-IR (Thermo Fisher Scientific, Waltham, MA) in 1×PBS for 20 minutes at 4° C. following a manufacturer's recommended procedure to discriminate live and dead cells. Cells were washed with 2% FBS (w/v) in 1×PBS (flow cytometry staining buffer) by centrifugation with benchtop Centrifuge 5810R (Eppendorf®, Hamburg, Germany). Cells were incubated for 30 minutes at 4° C. with serial dilutions of following antibodies ranging from 1.7 pM to 100 nM in flow cytometry staining buffer: (1) anti-CD40×CD4O bispecific antibodies, or (2) parental anti-CD40 antibodies (bivalent antibodies comprising 2 fragment antigen-binding [Fab] arms identical to one of the two anti-CD40 Fab of bispecific anti-CD40×CD4O antibodies), or (3) anti-CD40×Irrelevant antibodies (bivalent antibodies that incorporate one Fab arm identical to one of anti-CD40 Fab of anti-CD40×CD4O antibodies and another Fab arm specific to an irrelevant antigen, i.e. birch pollen main allergen Bet v1), or (4) human IgG4 isotype control antibodies with Fc mutation. After washing, cell bound antibodies were detected with 2.5 g/ml Allophycocyanin (APC)-conjugated goat anti-human IgG antibody specific for the Fcγ fragment (Jackson Immunoresearch, West Grove, PA) for 30 minutes at 4° C. Cells were washed and subsequently fixed with 50% solution of Cytofix™ Fixation Buffer (BD, Franklin Lakes, NJ) in flow cytometry staining buffer for 20 minutes at room temperature. Cells were washed and resuspended in flow cytometry staining buffer and stored at 4° C. for downstream flow cytometry analysis.

Acquisition of fluorescence signals were recorded on the ZE5 Cell Analyzer (Bio-Rad, Hercules, CA) according to the manufacturer's recommended procedure and flow cytometry data analysis was performed using the open-source R packages flowCore, flowStats, flowDensity, ggCyto, and flowAI. Multiplexed samples were deconvoluted and individual cell populations were identified based on their unique CellTrace™ fluorescence signature. APC Median Fluorescence intensity (MFI) was recorded to indicate the binding intensity of each antibody over a range of concentrations. Antibodies with MFI greater than 1500 at the highest tested concentration (100 nM) were classified as specific binders. In addition, direct binding signals (APC MFI) were analyzed as a function of the antibody concentration and data were fitted with a sigmoidal (four-parameter logistic) dose-response model using GraphPad Prism™ software. The EC₅₀ value, defined as the concentration of antibody that yields 50% of the maximal binding, was determined and used as an indicator of antibody binding potency. EC₅₀ values were reported only for specific binders.

### Results

The ability of anti-CD40×CD40 bispecific antibodies to bind specifically cells expressing human or monkey CD40 was assessed by flow cytometry. The experimental results are summarized in Table 21 below.

The four anti-CD40×CD40 bispecific antibodies (REGN16431, REGN16432, REGN16334, and REGN16335) displayed concentration-dependent specific binding to hCD40 expressed on HEK293/D9/hCD40 or Ramos 2G6.4C10 cells with MFI values ranging from 28,932 to 30,315 at the highest concentration tested (100 nM) and EC₅₀ values of 1.5 nM to 2.0 nM on HEK293/D9/hCD40 cells, or with MFI values from 3,748 to 3,852 and EC50 values of 1.0 nM to 2.0 nM on Ramos 2G6.4C10 cells. The four bispecific antibodies similarly displayed specific binding to mfCD40 expressed on HEK293/D9/mfCD40 cells with MFI values ranging from 41,714 to 48,653 and EC₅₀ values of 2.1 nM to 9.1 nM.

Six parental anti-CD40 antibodies (H4sH21519P2, H4sH21520P2, H4sH30027P2, REGN17288, REGN17544, and REGN17289) displayed specific binding to HEK293/D9/hCD40 or Ramos 2G6.4C10 cells with MFI values ranging from 20,213 to 20,911 and EC₅₀ values of 0.72 nM to 1.3 nM on HEK293/D9/hCD40 cells, or with MFI values ranging from 2,408 to 2,664 and EC₅₀ values of 0.45 nM to 1.5 nM on Ramos 2G6.4C10 cells. All parental anti-CD40 antibodies showed specific binding to HEK293/D9/mfCD40 cells with MFI values ranging from 11,539 to 33,930 and EC₅₀ values of 1.0 nM to 10 nM.

Six anti-CD40×Irrelevant antibodies (REGN17551, REGN17552, REGN17553, REGN17548, REGN17549, and REGN17550) displayed specific binding to HEK293/D9/hCD40 or Ramos 2G6.4C10 cells with MFI values ranging from 21,394 to 24,591 and EC₅₀ values of 1.1 nM to 8.2 nM on HEK293/D9/hCD40 cells, or with MFI values ranging from 1,833 to 3,140 on Ramos 2G6.4C10 cells. Four anti-CD40×Irrelevant antibodies (REGN17552, REGN17553, REGN17549, and REGN17550) displayed specific binding to HEK293/D9/mfCD40 cells with MFI values ranging from 4,719 to 39,448, but two anti-CD40×Irrelevant antibodies (REGN17551 and REGN17548) did not specifically bind to mfCD40 on HEK293/D9/mfCD40 cells.

All tested anti-CD40 antibodies did not bind to the negative control HEK293/D9 cells: binding signals were below 950 MFI. As expected, no detectable cell-surface binding was observed for two hIgG4 with Fc mutation isotype control antibodies (REGN7540 and REGN4513) up to the highest antibody concentration tested (100 nM).

TABLE 21
Summary of anti-CD40 antibody binding to human or monkey CD40 expressed on the cell surface.

| | | | | Ramos 2G6.4C10 | | | | HEK293/D9 | |
|---|---|---|---|---|---|---|---|---|---|
| | | HEK293/D9/hCD40 | | (hCD40 positive) | | HEK293/D9/mfCD40 | | (CD40 negative) | |
| | | EC₅₀ | MFI at | EC₅₀ | MFI at | EC₅₀ | MFI at | EC₅₀ | MFI at |
| Antibody ID | Antibody Format | (M) | 100 nM | (M) | 100 nM | (M) | 100 nM | (M) | 100 nM |
| REGN16431 | CD40 × CD40 | 1.9E−09 | 29464 | 1.9E−09 | 3851 | 8.4E−09 | 43145 | ND | 929 |
| REGN16432 | CD40 × CD40 | 1.5E−09 | 30315 | 1.1E−09 | 3852 | 2.1E−09 | 48653 | ND | 949 |
| REGN16334 | CD40 × CD40 | 2.0E−09 | 29042 | 2.0E−09 | 3759 | 9.1E−09 | 41714 | ND | 940 |
| REGN16335 | CD40 × CD40 | 1.6E−09 | 28932 | 1.0E−09 | 3748 | 2.4E−09 | 47289 | ND | 883 |
| H4sH21519P2 | Parental CD40 | 1.3E−09 | 20213 | 1.5E−09 | 2408 | 1.0E−08 | 11539 | ND | 434 |
| H4sH21520P2 | Parental CD40 | 8.2E−10 | 20681 | 8.0E−10 | 2446 | 3.7E−09 | 24090 | ND | 558 |
| H4sH30027P2 | Parental CD40 | 7.2E−10 | 20911 | 4.5E−10 | 2623 | 1.0E−09 | 33930 | ND | 650 |
| REGN17288 | Parental CD40 | 1.2E−09 | 20334 | 1.3E−09 | 2422 | 9.2E−09 | 11781 | ND | 488 |
| REGN17544 | Parental CD40 | 9.8E−10 | 20511 | 8.7E−10 | 2445 | 4.3E−09 | 23174 | ND | 599 |
| REGN17289 | Parental CD40 | 7.6E−10 | 20472 | 4.7E−10 | 2664 | 1.1E−09 | 32927 | ND | 618 |
| REGN17551 | CD40 × Irrelevant | 7.5E−09 | 23314 | INC | 2725 | ND | 1364 | ND | 328 |
| REGN17552 | CD40 × Irrelevant | 7.0E−09 | 21394 | INC | 1833 | INC | 4719 | ND | 433 |
| REGN17553 | CD40 × Irrelevant | 1.1E−09 | 24591 | 9.5E−10 | 3140 | 1.4E−9 | 39448 | ND | 822 |
| REGN17548 | CD40 × Irrelevant | 8.2E−09 | 23974 | INC | 2732 | ND | 1126 | ND | 311 |
| REGN17549 | CD40 × Irrelevant | 7.5E−09 | 22619 | INC | 2004 | INC | 5633 | ND | 471 |
| REGN17550 | CD40 × Irrelevant | 1.2E−09 | 24376 | 8.7E−10 | 3026 | 1.6E−09 | 39131 | ND | 776 |
| REGN7540 | Human IgG4 with Fc mutation | ND | 218 | ND | 74 | ND | 352 | ND | 181 |
| | Isotype control 1 | | | | | | | | |
| REGN4513 | Human IgG4 with Fc mutation | ND | 24 | ND | 42 | ND | 22 | ND | 21 |
| | Isotype control 2 | | | | | | | | |

MFI, median fluorescence intensity; INC, inconclusive; ND, not determined. INC is applied if concentration-dependent binding was detected, but no top plateau was reached in the concentraton range tested, and an EC₅₀ therefore could not be calculated. If MFI values were less than or equal to 1500 and specifidc binding was not detected, EC₅₀ was not calculated and ND was applied.

### Example 6. Bioassay to Assess Regulation of CD40 Signaling by Anti-CD40×CD40 Bispecific Antibodies

CD40 is a member of the tumor necrosis factor receptor superfamily (TNFRSF) that activates the immune system in response to the binding of its ligand, CD40L. To evaluate regulation of CD40 signaling, a bioassay was developed to quantitatively assess receptor activation by measuring gene expression downstream of the nuclear translocation of NFκB (nuclear factor KB). The luciferase-based reporter assay was engineered in three different human cell lines (Ramos.2G6.4C10, Raji, and THP-1) that endogenously express CD40. Cells were transduced with NFκB-luciferase reporter lentivirus (QIAGEN CLS-013L-8) and stable reporter cell lines were selected and maintained in media containing 1 μg/ml of puromycin.

For the bioassay, cells were seeded at 20,000 cells/well into 96-well plates in assay media (RPMI-1640 with 10% FBS, pen/strep/glut). Antibodies were then serially diluted in assay media at 1:3 to final concentrations ranging from 100 nM to 1.7 pM (with an additional condition without test molecule) and added to the cells along with or without a constant concentration of human CD40L (500 pM, 900 pM, or 10 nM for Ramos, Raji, or THP1 reporter cells, respectively). To obtain a range of activation, hCD40L was serially diluted 1:3 to final concentrations ranging from 100 nM to 1.7 pM (with an additional condition without ligand) and added to cells. After 5 hours of incubation at 37° C./5% CO₂, luciferase activity was detected on an Envision multilabel plate reader (PerkinElmer) after the addition of ONE-Glo™ (Promega) reagent. All conditions were tested in duplicate.

The EC₅₀ or IC₅₀ values were determined with GraphPad Prism™ software using nonlinear regression (4-parameter logistics). The percentage of inhibition was calculated based on the relative luminescence unit (RLU) values using the equation:

$$\% Activation = 100 \% \times \frac{{RLU}_{Antibody Maximum} - {RLU}_{Background}}{{RLU}_{Ligand Maximum} - {RLU}_{Background}}$$

$$\% Inhibition = 100 \% \times \frac{{RLU}_{Ligand Constant} - {RLU}_{Antibody Minimum}}{{RLU}_{Ligand Constamt} - {RLU}_{Background}}$$

“RLU_{Antibody Maximum}” and “RLU_{Antibody minimum}” are the maximum and minimum luminescence value achieved with antibody. “RLU_{Ligand Maximum}” and “RLU_{Ligand constant}” are the values achieved by maximum and constant concentration of CD40L. “RLU_{Background}” is the value without any CD40L. For antibodies that showed potentiation in the presence of CD40L, “RLU_{Antibody Maximum}” was used to calculate % inhibition leading to negative inhibition values.

### Results

As shown in Table 22, anti-CD40×CD40 bispecific antibodies REGN16431, REGN16432, REGN16334 and REGN16335 showed minimal activation ranging from 4 to 6% without CD40L and inhibition ranging from 90 to 94% with IC₅₀s of 38.6-85.4 pM with 500 pM CD40L in Ramos.2G6.4C10/NFκB-luc cells. Anti-CD40 bivalent antibodies and anti-CD40×Irrel. (Irrelevant, Non-CD40 target) antibodies showed activation ranging from 4 to 49% without CD40L and inhibition ranging from 28 to 95% with 500 pM CD40L. Four antibodies showed potentiation of signaling, inhibition ranging from −110 to −142% in the presence of CD40L in Ramos bioassay.

As shown in Table 23, anti-CD40×CD40 bispecific antibodies REGN16431, REGN16432, REGN16334 and REGN16335 showed minimal activation ranging from 5 to 13% without CD40L and inhibition ranging from 68 to 113% with IC₅₀s of 49.4-107 pM with 900 pM CD40L in Raji/NFκB-luc cells. Anti-CD40 bivalent antibodies and anti-CD40×Irrel. antibodies showed activation ranging from 7 to 25% without CD40L and inhibition ranging from 33 to 92% with 900 pM CD40L in Raji bioassay.

As shown in Table 24, anti-CD40×CD40 bispecific antibodies REGN16431, REGN16432, REGN16334 and REGN16335 showed minimal activation ranging from 1 to 6% without CD40L and inhibition ranging from 97 to 98% with IC₅₀s of 148-553 pM with 10 nM CD40L in THP-1/NFκB-luc cells. Anti-CD40 bivalent antibodies and anti-CD40×Irrel. antibodies showed activation ranging from 0 to 5% without CD40L and inhibition ranging from 54 to 101% with 10 nM CD40L. Four antibodies showed potentiation of signaling, inhibition ranging from −45 to −92%, in the presence of CD40L in THP-1 bioassay.

Control mAb1, Control mAb2 and Control mAb3, irrelevant human IgG antibodies, showed little to no activation (0-13%) without CD40L and inhibition (3-23%) with CD40L in all cells. CD40L showed activation of signaling with EC₅₀s of 436 pM, 691 pM and 1.47 nM in Ramos.2G6.4C10/NFκB-luc, Raji//NFκB-luc, and THP-1/NFκB-luc cells, respectively.

TABLE 22
Anti-CD40 × CD40 bispecific antibody regulation of CD40 signaling in
the presence or absence of human CD40L using Ramos.2G6.4C10/NFκB-luc cells.

| | | | | Regulation with 500 pM | |
|---|---|---|---|---|---|
| | | Regulation with No Ligand | | hCD40L | |
| | | | Max % | IC50/EC50 | Max % |
| Antibody | Specificity | EC50 [M] | activation | [M] | inhibition |
| REGN16431 | CD40 × CD40 | No Activation | 6 | 8.54E−11 | 90 |
| | (21519 × 21520) | | | | |
| REGN16432 | CD40 × CD40 | No Activation | 4 | 4.92E−11 | 94 |
| | (30027 × 21520) | | | | |
| REGN16334 | CD40 × CD40 | No Activation | 6 | 6.93E−11 | 91 |
| | (21519 × 21520) | | | | |
| REGN16335 | CD40 × CD40 | No Activation | 5 | 3.86E−11 | 93 |
| | (30027 × 21520) | | | | |
| H4sH21519P2 | CD40 (21519) | No Activation | 13 | 3.09E−11 | 88 |
| H4sH21520P2 | CD40 (21520) | >1.00E−08 | 49 | 8.06E−11# | 63 |
| H4sH30027P2 | CD40 (30027) | No Activation | 8 | 8.14E−12* | −130 |
| REGN17288 | CD40 (21519) | No Activation | 6 | 4.07E−11 | 88 |
| REGN17544 | CD40 (21520) | No Activation | 10 | 1.89E−10 | 77 |
| REGN17289 | CD40 (30027) | No Activation | 4 | 7.51E−12* | −133 |
| REGN17548 | CD40 × Irrel. | No Activation | 4 | 1.05E−08 | 95 |
| | (21519 × Irrel.) | | | | |
| REGN17549 | CD40 × Irrel. | >1.00E−08 | 25 | >1.00E−08 | 28 |
| | (21520 × Irrel.) | | | | |
| REGN17550 | CD40 × Irrel. | No Activation | 5 | 2.45E−11* | −142 |
| | (30027 × Irrel.) | | | | |
| REGN17551 | CD40 × Irrel. | No Activation | 5 | 7.29E−09 | 95 |
| | (21519 × Irrel.) | | | | |
| REGN17552 | CD40 × Irrel. | >1.00E−08 | 20 | >1.00E−08 | 51 |
| | (21520 × Irrel.) | | | | |
| REGN17553 | CD40 × Irrel. | No Activation | 4 | 2.63E−11* | −110 |
| | (30027 × Irrel.) | | | | |
| Control Mab1 | Irrel. | No Activation | 4 | No Inhibition | 5 |
| Control mAb2 | Irrel. | No Activation | 4 | No Inhibition | 3 |
| Control mAb3 | Irrel. | No Activation | 2 | No Inhibition | 12 |

*Values represent EC₅₀ calculations in the presence of CD40L. All other values are IC₅₀s.
#IC₅₀ values was obtained by excluding RLU values from conditions containing three highest concentrations of antibody due to hook effect.

TABLE 23
Anti-CD40 × CD40 bispecific antibody regulation of CD40 signaling in
the presence or absence of human CD40L using Raji/NFκB-luc cells.

| | | | | Regulation with 900pM | |
|---|---|---|---|---|---|
| | | Regulation with No Ligand | | hCD40L | |
| | | | Max % | | Max % |
| Antibody | Specificity | EC50 [M] | activation | IC50 [M] | inhibition |
| REGN16431 | CD40 × CD40 | No Activation | 13 | 1.07E−10 | 82 |
| | (21519 × 21520) | | | | |
| REGN16432 | CD40 × CD40 | No Activation | 5 | 1.02E−10 | 68 |
| | (30027 × 21520) | | | | |
| REGN16334 | CD40 × CD40 | No Activation | 13 | 5.76E−11 | 113 |
| | (21519 × 21520) | | | | |
| REGN16335 | CD40 × CD40 | No Activation | 9 | 4.94E−11 | 102 |
| | (30027 × 21520) | | | | |
| H4sH21519P2 | CD40 (21519) | No Activation | 15 | 6.05E−11 | 92 |
| H4sH21520P2 | CD40 (21520) | >1.00E−07 | 25 | 7.70E−11 | 81 |
| H4sH30027P2 | CD40 (30027) | No Activation | 9 | 8.95E−12 | 67 |
| REGN17288 | CD40 (21519) | No Activation | 14 | 4.28E−11 | 90 |
| REGN17544 | CD40 (21520) | No Activation | 11 | 1.35E−10 | 81 |
| REGN17289 | CD40 (30027) | No Activation | 10 | 2.72E−11 | 55 |
| REGN17548 | CD40 × Irrel. | No Activation | 9 | >1.00E−08 | 74 |
| | (21519 × Irrel.) | | | | |
| REGN17549 | CD40 × Irrel. | No Activation | 11 | >1.00E−08 | 52 |
| | (21520 × Irrel.) | | | | |
| REGN17550 | CD40 × Irrel. | No Activation | 14 | 2.99E−10 | 33 |
| | (30027 × Irrel.) | | | | |
| REGN17551 | CD40 × Irrel. | No Activation | 14 | >1.00E−08 | 58 |
| | (21519 × Irrel.) | | | | |
| REGN17552 | CD40 × Irrel. | No Activation | 14 | >1.00E−08 | 56 |
| | (21520 × Irrel.) | | | | |
| REGN17553 | CD40 × Irrel. | No Activation | 7 | 1.39E−10 | 37 |
| | (30027 × Irrel.) | | | | |
| Control Mab1 | Irrel. | No Activation | 6 | No Inhibition | 23 |
| Control mAb2 | Irrel. | No Activation | 9 | No Inhibition | 19 |
| Control mAb3 | Irrel. | No Activation | 13 | No Inhibition | 16 |

TABLE 24
Anti-CD40 × CD40 bispecific antibody regulation of CD40 signaling in
the presence or absence of human CD40L using THP-1/NFκB-luc cells.

| | | Regulation with No Ligand | | Regulation with 10nM hCD40L | |
|---|---|---|---|---|---|
| | | | Max % | | Max % |
| Antibody | Specificity | EC50 [M] | activation | IC50/EC50 [M] | inhibition |
| REGN16431 | CD40 × CD40 | No Activation | 2 | 2.87E−10 | 98 |
| | (21519 × 21520) | | | | |
| REGN16432 | CD40 × CD40 | No Activation | 6 | 1.48E−10 | 97 |
| | (30027 × 21520) | | | | |
| REGN16334 | CD40 × CD40 | No Activation | 1 | 5.53E−10 | 98 |
| | (21519 × 21520) | | | | |
| REGN16335 | CD40 × CD40 | No Activation | 3 | 2.21E−10 | 98 |
| | (30027 × 21520) | | | | |
| H4sH21519P2 | CD40 (21519) | No Activation | 5 | 2.76E−10 | 99 |
| H4sH21520P2 | CD40 (21520) | No Activation | 1 | 4.37E−10 | 97 |
| H4sH30027P2 | CD40 (30027) | No Activation | 2 | 2.51E−12* | −61 |
| REGN17288 | CD40 (21519) | No Activation | 0 | 3.34E−10 | 98 |
| REGN17544 | CD40 (21520) | No Activation | 4 | 1.13E−09 | 96 |
| REGN17289 | CD40 (30027) | No Activation | 1 | 1.60E−11* | −92 |
| REGN17548 | CD40 × Irrel. | No Activation | 2 | 1.51E−08 | 97 |
| | (21519 × Irrel.) | | | | |
| REGN17549 | CD40 × Irrel. | No Activation | 1 | >1.00E−08 | 54 |
| | (21520 × Irrel.) | | | | |
| REGN17550 | CD40 × Irrel. | No Activation | 1 | 4.64E−11* | −74 |
| | (30027 × Irrel.) | | | | |
| REGN17551 | CD40 × Irrel. | No Activation | 1 | 4.55E−09 | 101 |
| | (21519 × Irrel.) | | | | |
| REGN17552 | CD40 × Irrel. | No Activation | 3 | >1.00E−08 | 83 |
| | (21520 × Irrel.) | | | | |
| REGN17553 | CD40 × Irrel. | No Activation | 1 | Not | −45 |
| | (30027 × Irrel.) | | | Determined* | |
| Control Mab1 | Irrel. | No Activation | 0 | No Inhibition | 13 |
| Control mAb2 | Irrel. | No Activation | 1 | No Inhibition | 4 |
| Control mAb3 | Irrel. | No Activation | 2 | No Inhibition | 5 |

*Values represent EC₅₀ calculations in the presence of CD40L. EC₅₀ value was not determined where best-fit value was not found. All other values are IC₅₀s.

### Example 7. In Vitro CD40L Blocking Activity by Anti-CD40×CD40 Bispecific Antibodies

### B Cell Assay #1

To determine the efficacy of anti-CD40×CD40 bispecific antibodies to block stimulation by CD40L, IL-6, IL-10, and TNFα cytokine production was quantified in primary human B cell cultures treated with antibodies in the presence of soluble CD40L. B cells were plated at 2×10⁵ cells per well in a 96 U-bottom plate in RPMI 1640 media with 15% FBS and 1× penicillin-streptomycin. Anti-CD40×CD40 bispecific antibodies were added to cells simultaneously in the presence of a constant dose of IL-4 (10 pM) and CD40L (500 nM). A dose response of CD40L with a constant dose of IL-4 (10 pM) was included as a control. Cells were cultured for 3 days at 37° C., and cell culture supernatant was collected for analysis.

Cytokine levels in supernatant were analyzed using the MSD V-PLEX proinflammatory panel 1. MSD V-PELX assay was performed according to the manufacturer's instruction using cell culture supernatant diluted 1:2. MSD plates were read using an MESO QuickPlex Sq 120MM instrument and MSD Discovery Workbench software. Data analysis was performed using Graphpad Prism software. The data points obtained were transformed using an X=Log(X) equation, and the transformed data were subjected to a linear regression analysis and fitted into a sigmoidal dose response curve. IC₅₀s were derived from this analysis.

### Results

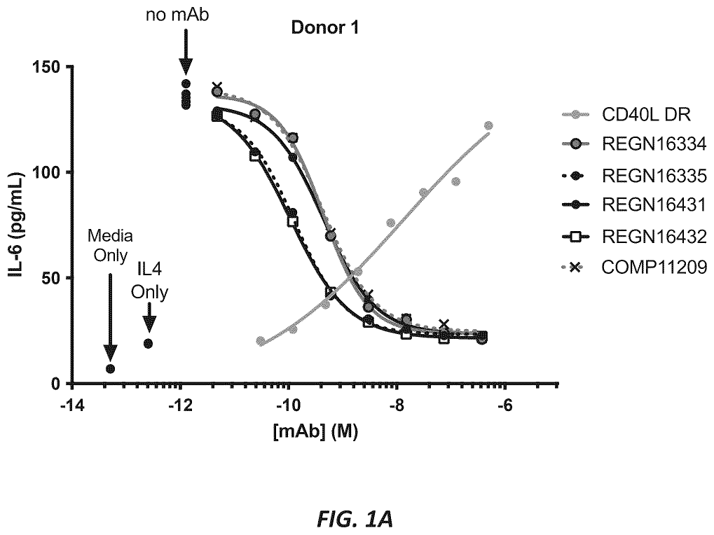

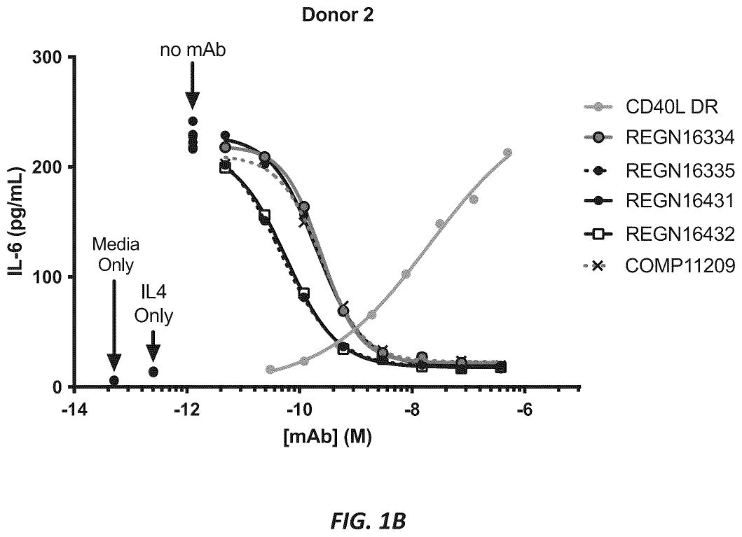

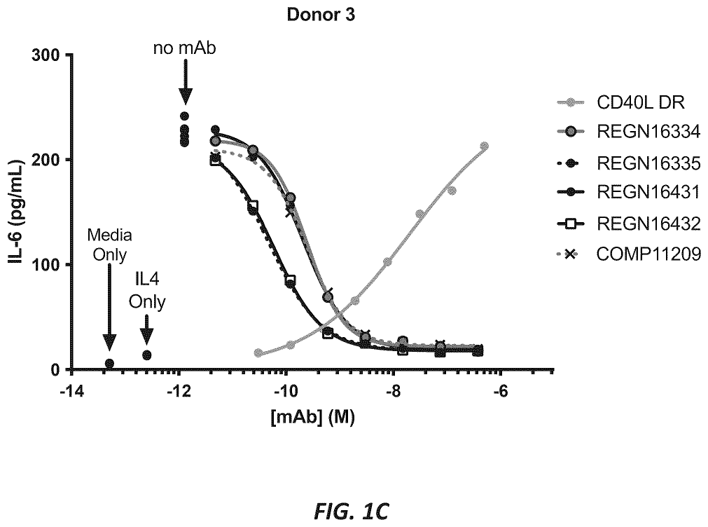

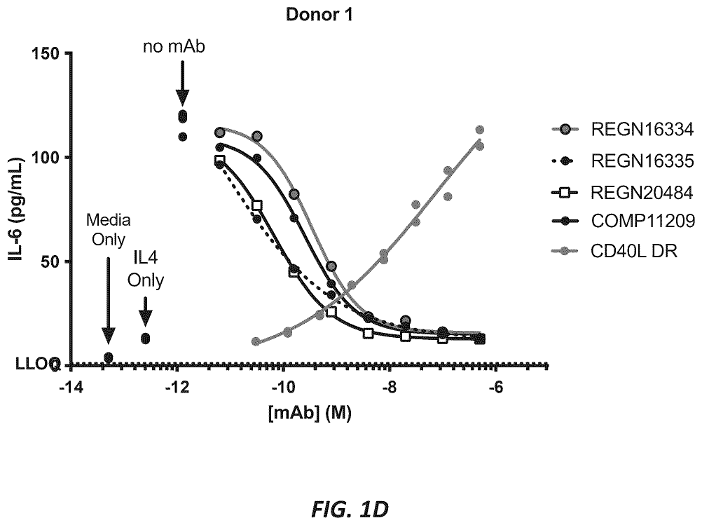

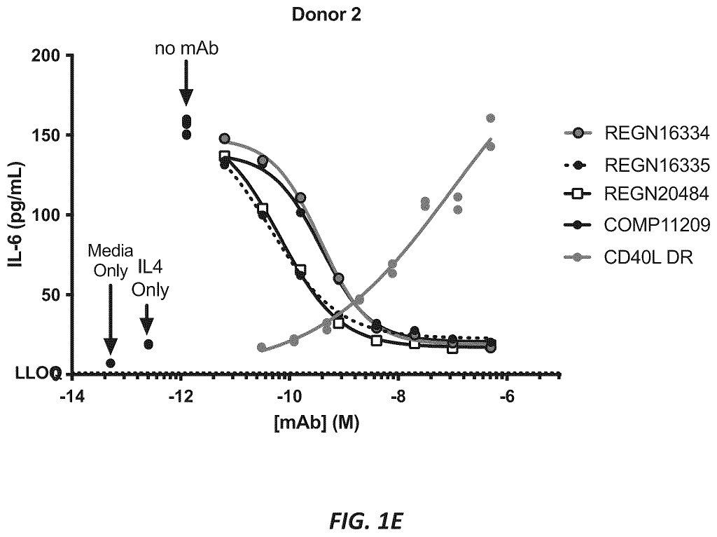

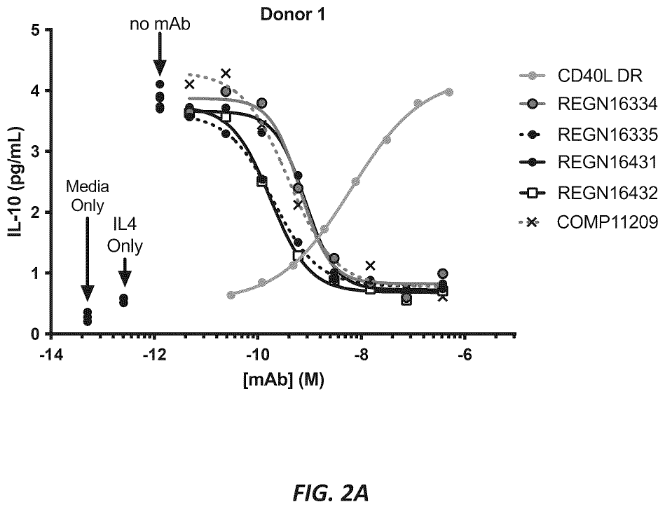

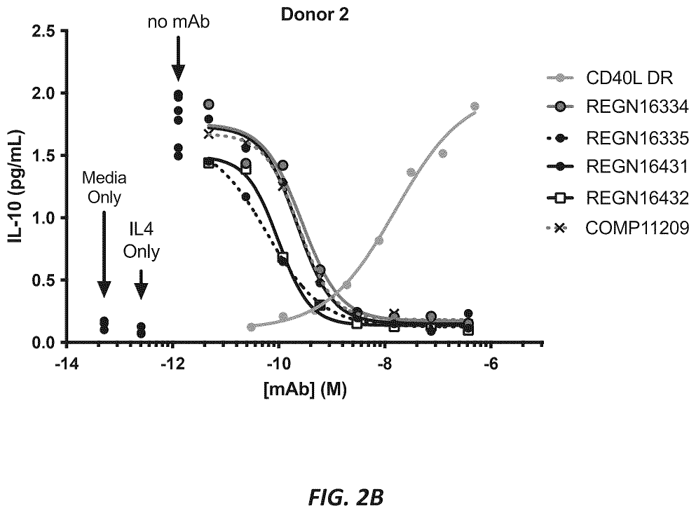

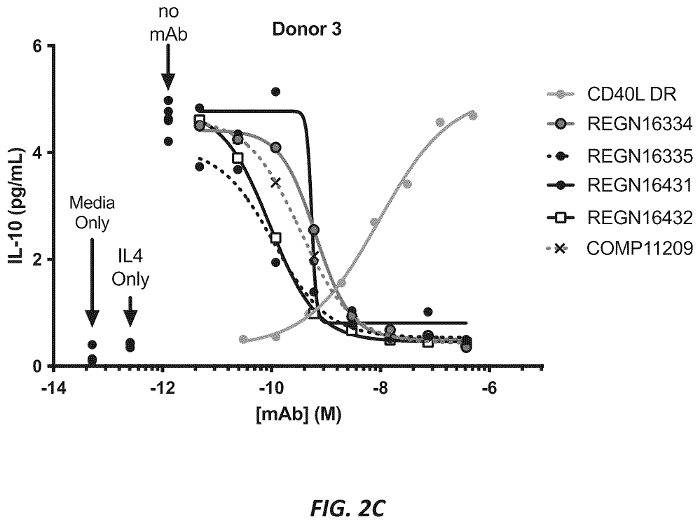

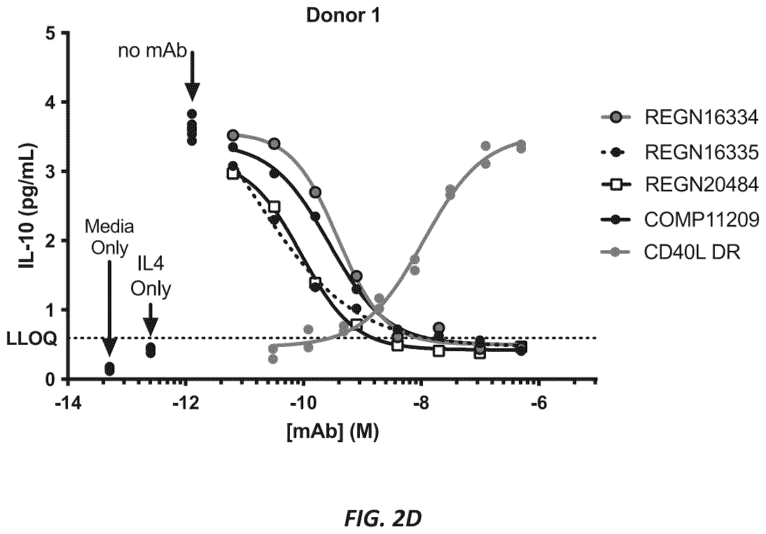

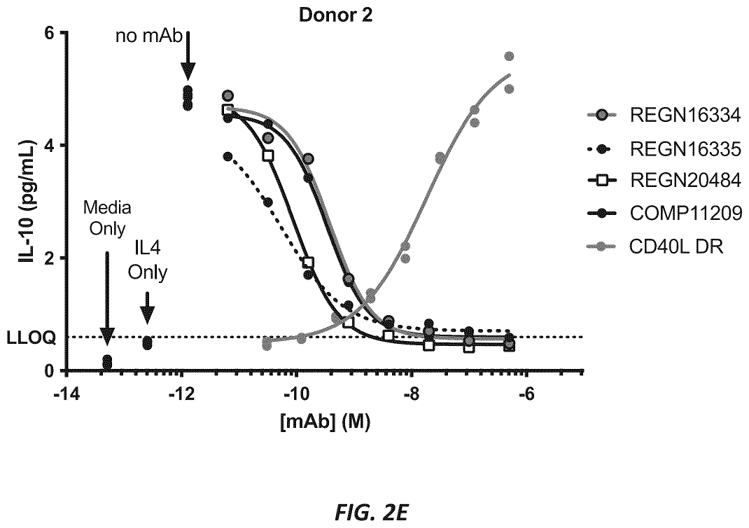

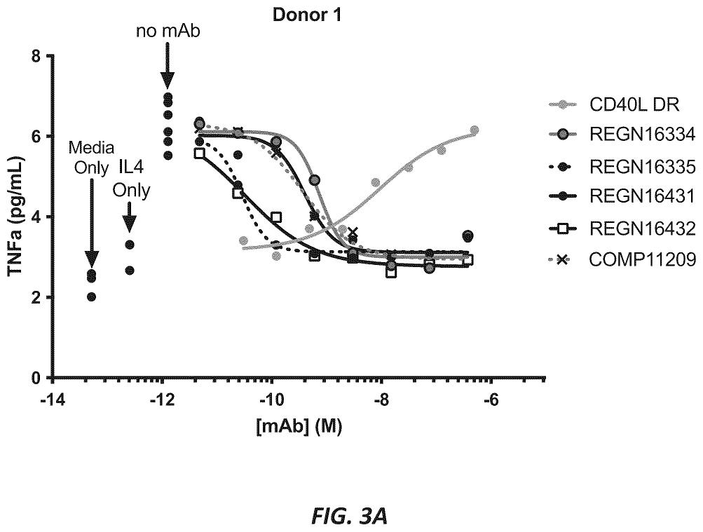

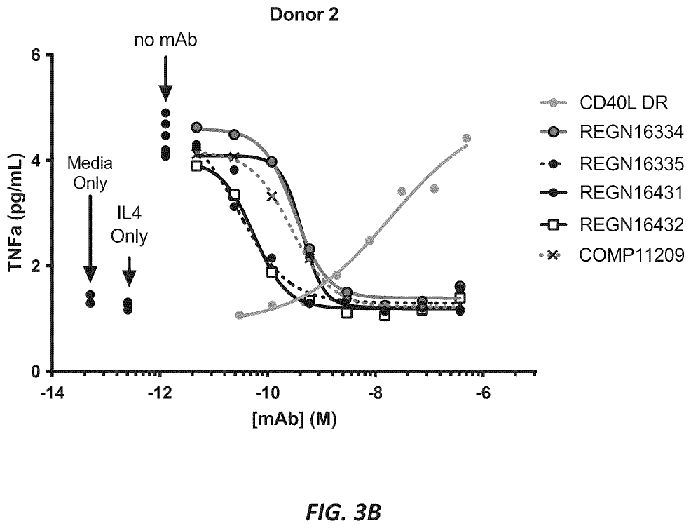

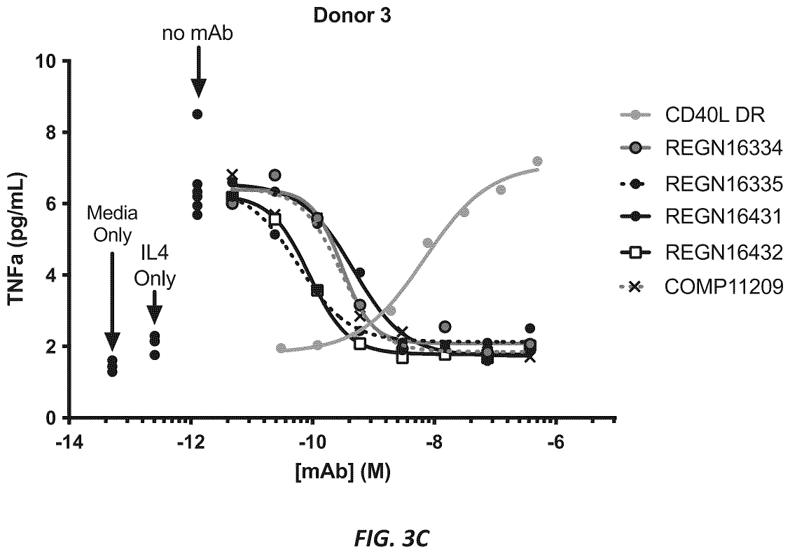

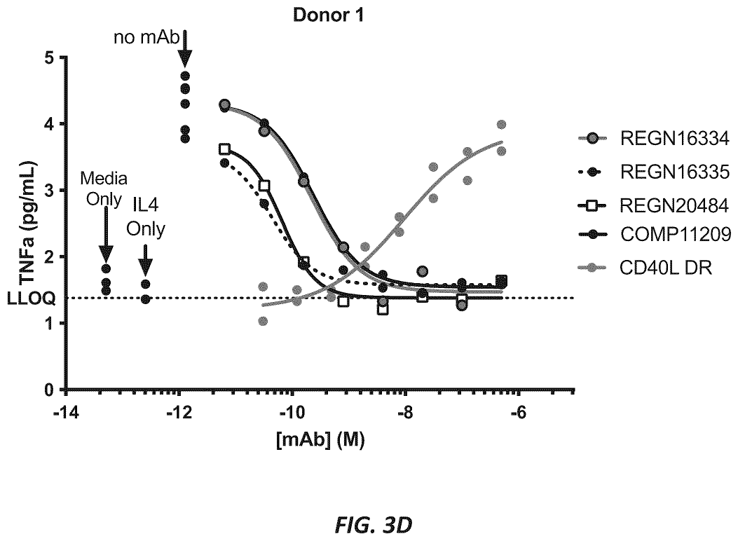

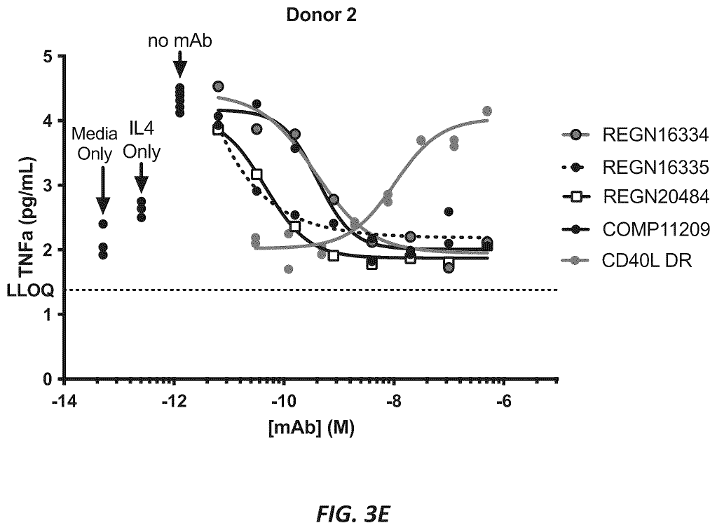

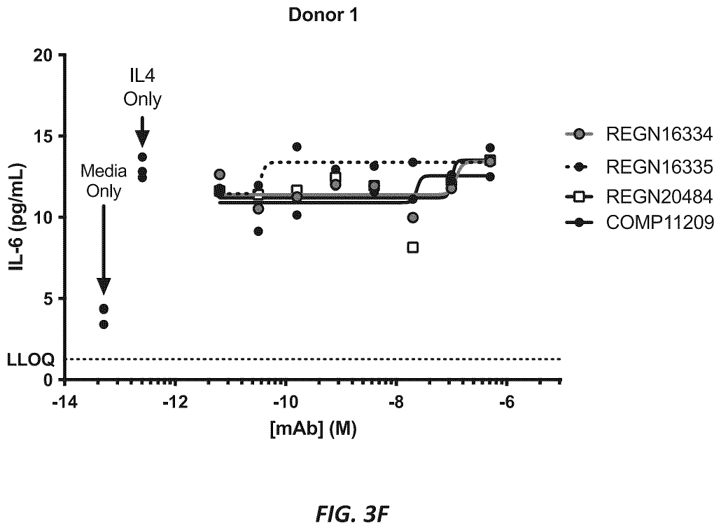

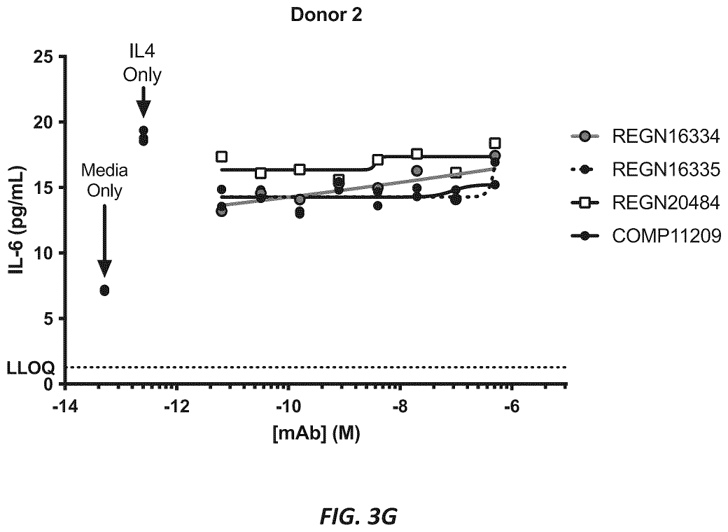

All four antibodies tested (REGN16334, REGN16335, REGN16431, and REGN16432) blocked IL-6 (FIGS. 1A, 1B, and 1C), IL-10 (FIGS. 2A, 2B, and 2C), and TNFa (FIGS. 3A, 3B, and 3C) production from primary human B cells from three donors in response to stimulation with CD40L. IC₅₀ values of antibodies and COMP11209 (a comparator anti-CD40 antibody) are shown in Tables 25 (IL-6), 26 (IL-10), and 27 (TNFα). The percent max blockade was calculated with the highest dose of REGN16334, REGN16335, REGN16431, and REGN16432, relative to the average cytokine levels in cells treated with no antibody.

TABLE 25
Effect of anti-CD40 × CD40 bispecific antibodies on
human B cell IL-6 production in the presence of constant CD40L.

| | | Donor 1% | | Donor 2% | | Donor 3% |
|---|---|---|---|---|---|---|
| | | max | | max | | max |
| | | blockade | | blockade | | blockade |
| | | relative to | | relative to | | relative to |
| Antibody | IC₅₀ Donor 1 | no mAb | IC₅₀ Donor 2 | no mAb | IC₅₀ Donor 3 | no mAb |
| REGN16334 | 4.400e−010 | 84.49% | 2.474e−010 | 92.00% | 2.474e−010 | 92.00% |
| REGN16335 | 1.281e−010 | 83.22% | 4.579e−011 | 91.36% | 4.579e−011 | 91.36% |
| REGN16431 | 4.488e−010 | 84.05% | 2.051e−010 | 92.66% | 2.051e−010 | 92.66% |
| REGN16432 | 1.081e−010 | 83.54% | 5.866e−011 | 92.04% | 5.866e−011 | 92.04% |
| COMP11209 | 4.341e−010 | 84.20% | 2.444e−010 | 91.32% | 2.444e−010 | 91.32% |

TABLE 26
Effect of anti-CD40 × CD40 bispecific antibodies on human
B cell IL-10 production in the presence of constant CD40L.

| | | Donor 1% | | Donor 2% | | Donor 3% |
|---|---|---|---|---|---|---|
| | | max | | max | | max |
| | | blockade | | blockade | | blockade |
| | | relative to | | relative to | | relative to |
| Antibody | IC₅₀ Donor 1 | no mAb | IC₅₀ Donor 2 | no mAb | IC₅₀ Donor 3 | no mAb |
| REGN16334 | 6.721e−010 | 74.23% | 2.759e−010 | 91.42% | 6.466e−010 | 92.42% |
| REGN16335 | 1.971e−010 | 78.54% | 6.116e−011 | 93.60% | 1.104e−010 | 89.80% |
| REGN16431 | 8.224e−010 | 80.68% | 2.242e−010 | 86.87% | ~5.481e−010 | 89.29% |
| REGN16432 | 1.767e−010 | 81.55% | 9.896e−011 | 94.47% | 9.371e−011 | 90.62% |
| COMP11209 | 3.894e−010 | 84.09% | 2.392e−010 | 91.67% | 3.399e−010 | 91.43% |

TABLE 27
Effect of anti-CD40 × CD40 bispecific antibodies on human B cell
TNFα production in the presence of constant CD40L.

| | | Donor 1% | | Donor 2% | | Donor 3% |
|---|---|---|---|---|---|---|
| | | max | | max | | max |
| | | blockade | | blockade | | blockade |
| | | relative to | | relative to | | relative to |
| Antibody | IC₅₀ Donor 1 | no mAb | IC₅₀ Donor 2 | no mAb | IC₅₀ Donor 3 | no mAb |
| REGN16334 | 7.374e−010 | 44.05% | 3.207e−010 | 63.52% | 3.047e−010 | 68.51% |
| REGN16335 | 2.888e−011 | 44.98% | 2.906e−011 | 64.66% | 5.743e−011 | 61.62% |
| REGN16431 | 3.989e−010 | 44.70% | 4.395e−010 | 74.13% | 4.711e−010 | 70.43% |
| REGN16432 | 3.291e−011 | 53.54% | 5.596e−011 | 68.40% | 8.796e−011 | 69.14% |
| COMP11209 | 3.667e−010 | 53.86% | 2.799e−010 | 71.40% | 2.838e−010 | 73.91% |

### B Cell Assay #2

To determine the efficacy of anti-CD40×CD40 bispecific antibodies to block stimulation by CD40L, IL-6, IL-10, and TNFa cytokine production was quantified in primary human B cell cultures treated with antibodies in the presence of soluble CD40L. B cells were plated at 2×10⁵ cells per well in a 96 U-bottom plate in RPMI 1640 media with 15% FBS and 1× penicillin-streptomycin and incubated with anti-CD40×CD40 bispecific antibodies for 30 minutes at 37° C. Following antibody incubation, a constant dose of IL-4 (10 pM) and CD40L (500 nM) was added to the B cell cultures in the presence of the anti-CD40×CD40 bispecific antibodies. A dose response of CD40L with a constant dose of IL-4 (10 pM) was included as a control. In parallel, to quantify the agonistic activity of anti-CD40×CD40 bispecific antibodies, B cells were incubated with antibodies in the presence of a constant dose of IL4 (10 pM) without CD40L. Cells were cultured for 3 days at 37° C., and cell culture supernatant was collected for analysis.

Cytokine levels in supernatant were analyzed using the MSD V-PLEX proinflammatory panel 1. MSD V-PELX assay was performed according to the manufacturer's instruction using cell culture supernatant diluted 1:2. MSD plates were read using an MESO QuickPlex Sq 120MM instrument and MSD Discovery Workbench software. Data analysis was performed using Graphpad Prism software. The data points obtained were transformed using an X=Log(X) equation, and the transformed data were subjected to a linear regression analysis and fitted into a sigmoidal dose response curve. IC₅₀s were derived from this analysis.

### Results

All three anti-CD40×CD40 bispecific antibodies tested (REGN16334, REGN16335, and REGN20484) blocked IL6 (FIGS. 1D and 1E), IL10 (FIGS. 2D and 2E), and TNFα (FIGS. 3D and 3E) cytokine production from primary human B cells from two donors in response to stimulation with CD40L. IC₅₀ values of antibodies and COMP11209 are shown in Tables 28-30. The percent max blockade was calculated with the highest dose of REGN16334, REGN16335, and REGN20484, relative to the average cytokine levels in cells treated with no antibody. Analysis of agonistic activity of antibodies REGN16334, REGN16335, and REGN20484 showed induction of the cytokine IL6 within a range comparable to cells treated with IL4 only (no antibody) in both donors (FIGS. 3F and 3G).

TABLE 28
Effect of anti-CD40 × CD40 bispecific antibodies on
human B cell IL-6 production in the presence of constant CD40L.

| | | Donor 1% max | | |
|---|---|---|---|---|
| | | blockade | | Donor 2% max |
| | | relative to no | | blockade relative |
| Antibody | IC₅₀ Donor 1 | mAb | IC₅₀ Donor 2 | to no mAb |
| REGN16334 | 3.505e−010 | 88.89% | 3.753e−010 | 89.15% |
| REGN16335 | 5.645e−012 | 88.41% | 4.403e−011 | 87.04% |
| REGN20484 | 6.741e−011 | 88.96% | 6.758e−011 | 88.10% |
| COMP11209 | 2.534e−010 | 88.59% | 3.806e−010 | 88.30% |

TABLE 29
Effect of anti-CD40xCD40 bispecific antibodies on human B cell IL-10
production in the presence of constant CD40L.

| | | Donor 1% max | | Donor 2% max |
|---|---|---|---|---|
| | | blockade | | blockade |
| | | relative to no | IC₅₀ | relative |
| Antibody | IC₅₀ Donor 1 | mAb | Donor 2 | to no mAb |
| REGN16334 | 3.874e−010 | 87.68% | 3.747e−010 | 89.92% |
| REGN16335 | 1.413e−011 | 88.54% | 5.451e−011 | 87.75% |
| REGN20484 | 9.206e−011 | 87.07% | 8.723e−011 | 90.92% |
| COMP11209 | 2.767e−010 | 88.08% | 3.360e−010 | 90.48% |

TABLE 30
Effect of anti-CD40xCD40 bispecific antibodies on human B
cell TNFα production in the presence of constant CD40L.

| | | Donor | | Donor |
|---|---|---|---|---|
| | | 1% max | | 2% max |
| | | blockade | | blockade |
| | | relative to no | IC₅₀ | relative |
| Antibody | IC₅₀ Donor 1 | mAb | Donor 2 | to no mAb |
| REGN16334 | 2.176e−010 | 62.82% | 3.776e−010 | 51.15% |
| REGN16335 | 4.396e−011 | 62.40% | ~2.967e−016 | 52.52% |
| REGN20484 | 6.632e−011 | 61.87% | 4.573e−011 | 52.27% |
| COMP11209 | 2.364e−010 | 62.03% | 4.054e−010 | 50.57% |

### Dendritic Cell Assay

To determine the efficacy of anti-CD40×CD40 bispecific antibodies to block stimulation by CD40L, IL-12/IL-23p40 cytokine production was quantified in primary human monocyte derived dendritic cell (MDDCs) cultures treated with antibodies in the presence of soluble CD40L. To generate MDDCs, peripheral blood mononuclear cells (PBMCs) from healthy human donors were isolated from leukopacks obtained from the New York Blood Center by Ficoll-Paque density gradient centrifugation. CD14+ cells were purified from PBMCs by positive selection using CD14 human microbeads. Purified CD14+ cells were plated at 3×10⁶ cells per well in a 6-well plate in RPMI 1640 media with 10% FBS, 1× penicillin-streptomycin, 800 U/mL GM-CSF, and 500 U/mL of IL-4. Media was replenished and the complete amount of GM-CSF and IL-4 was added back to cells on day 3 and day 5. On day 6 MDDCs were collected and plated at 1×10⁶ cells per well in a 96-U bottom plate. Anti-CD40×CD40 bispecific antibodies were added to cells simultaneously with a constant dose of CD40L (20 nM). A dose response of CD40L was included as a control. Cells were cultured for 4 days at 37° C., and cell culture supernatant was collected for analysis.

Cytokine levels in supernatant were analyzed using the MSD V-PLEX cytokine panel 1. MSD V-PLEX assay was performed according to the manufacturer's instruction using cell culture supernatant diluted at 1:2 or 1:20. MSD plates were read using an MESO QuickPlex Sq 120MM instrument and MSD Discovery Workbench software. Data analysis was performed using Graphpad Prism software. The data points obtained were transformed using an X=Log(X) equation, and the transformed data were subjected to a linear regression analysis and fitted into a sigmoidal dose response curve. IC₅₀s were derived from this analysis.

### Results

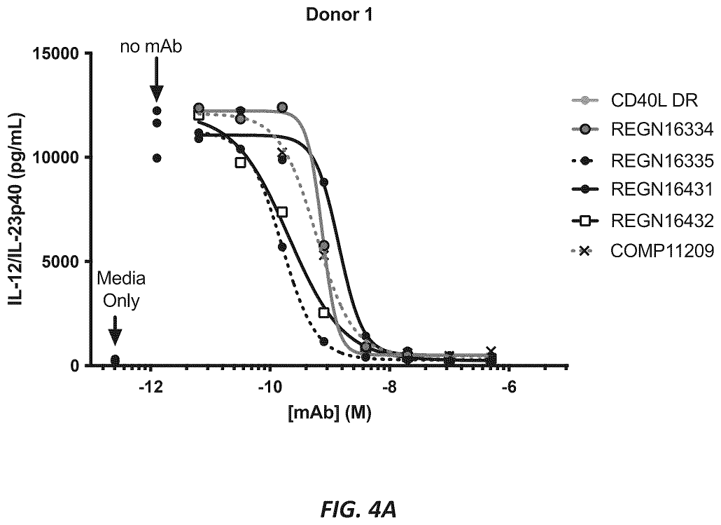

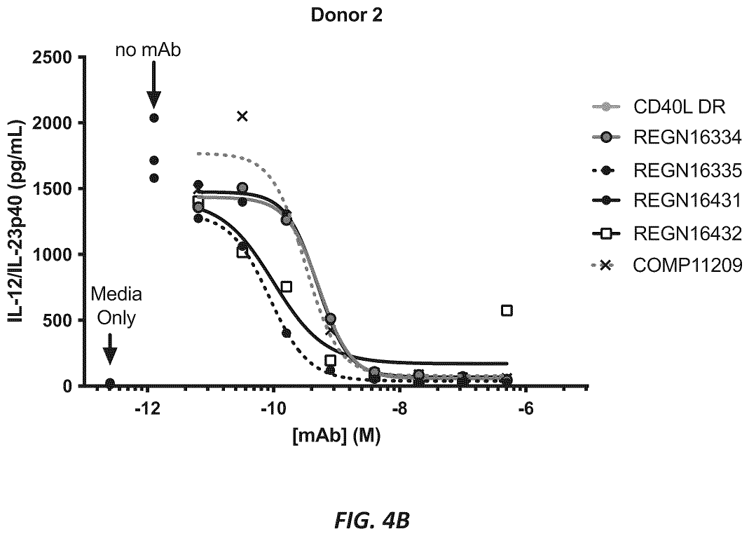

All four antibodies tested (REGN16334, REGN16335, REGN16431, and REGN16432) blocked IL-12/IL-23p40 production from primary human monocyte derived dendritic cells from two donors in response to stimulation with CD40L (FIGS. 4A-4B). IC₅₀ values of antibodies and COMP11209 are shown in Table 31.

TABLE 31
Effect of anti-CD40xCD40 bispecific antibodies on human dendritic
cell IL-12/IL-23p40 production in the presence of constant CD40L.

| Antibody | IC₅₀ Donor 1 | IC₅₀ Donor 2 |
|---|---|---|
| REGN16334 | 7.533e−010 | 5.275e−010 |
| REGN16335 | 1.576e−010 | 8.547e−011 |
| REGN16431 | 1.422e−009 | 4.972e−010 |
| REGN16432 | 2.064e−010 | 9.919e−011 |
| COMP11209 | 5.938e−010 | 3.191e−010 |

### Example 8. In Vitro Agonist Activity in Human B Cells by Anti-CD40×CD40 Bispecific Antibodies

To quantify the agonistic activity of anti-CD40×CD40 bispecific antibodies, B cells were incubated with antibodies in the presence of a constant dose of IL-4 (10 pm) without CD40L. Cells were cultured for 3 days at 37° C., and cell culture supernatant was collected for analysis. Cytokine levels in supernatant were analyzed using the MSD V-PLEX proinflammatory panel 1. MSD V-PELX assay was performed according to the manufacturer's instruction using cell culture supernatant diluted 1:2. MSD plates were read using an MESO QuickPlex Sq 120MM instrument and MSD Discovery Workbench software. Data analysis was performed using Graphpad Prism software. The data points obtained were transformed using an X=Log(X) equation, and the transformed data were subjected to a linear regression analysis and fitted into a sigmoidal dose response curve. IC₅₀s were derived from this analysis.

### Results

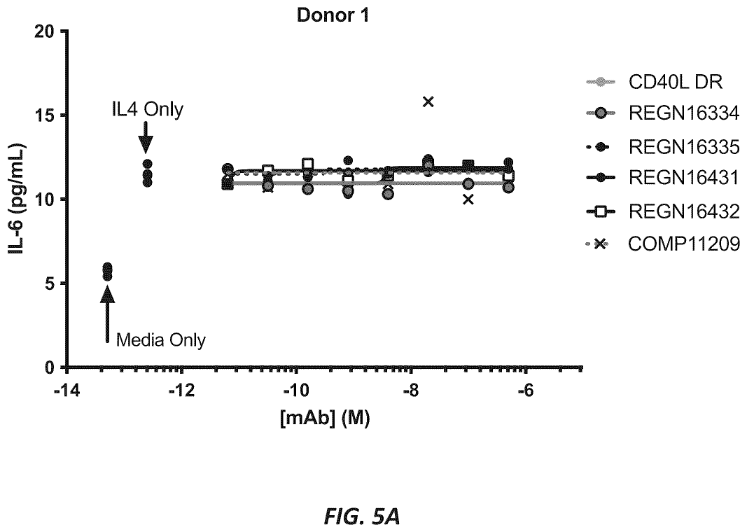

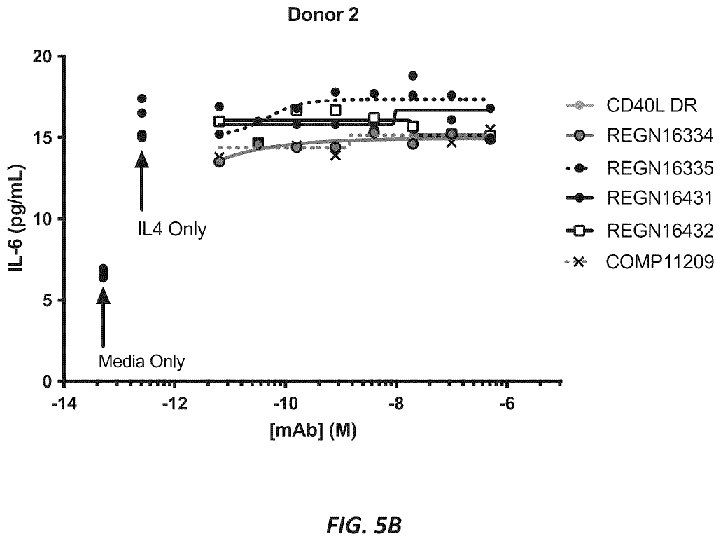

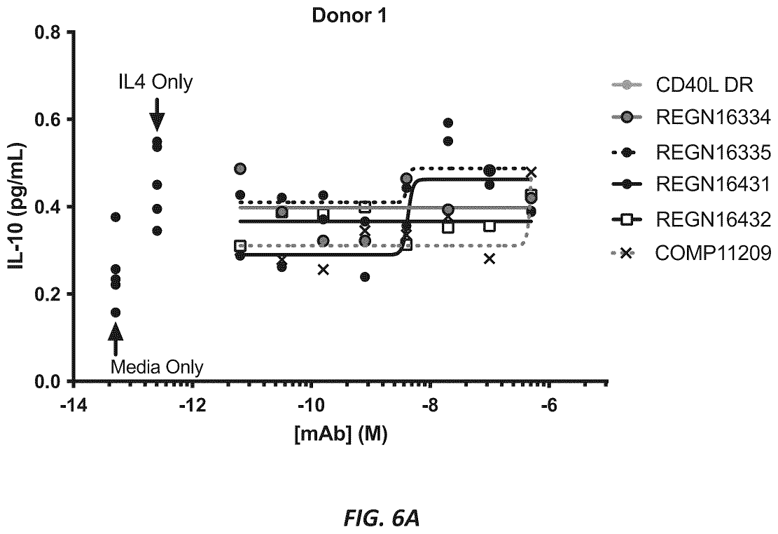

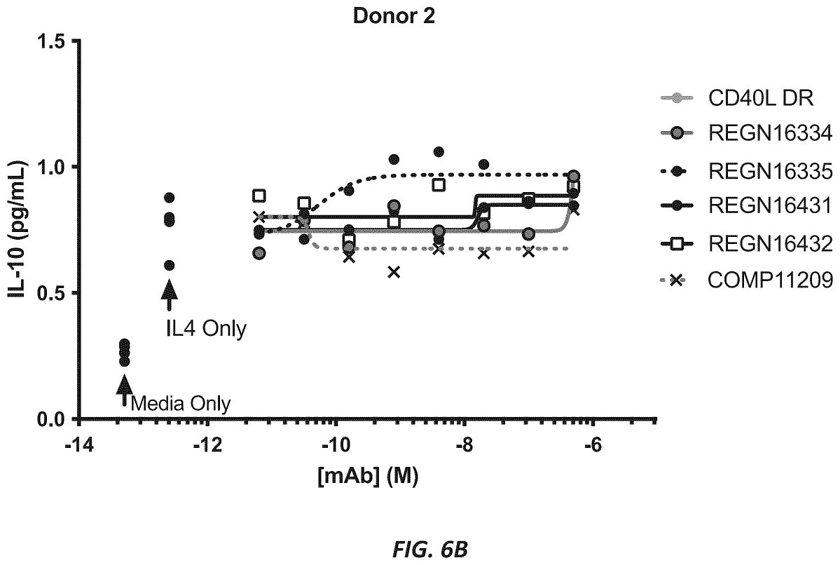

Analysis of the agonistic activity of antibodies REGN16334, REGN16335, REGN16431, and REGN16432 showed induction of the cytokines IL-6 (FIGS. 5A-5B) and IL-10 (FIGS. 6A-6B) within a range comparable to cells treated with no antibody (IL-4 only) in both donors.

### Example 9. Impact of Anti-CD40×CD40 Bispecific Antibodies in a Mouse NP-KLH Immunization Model

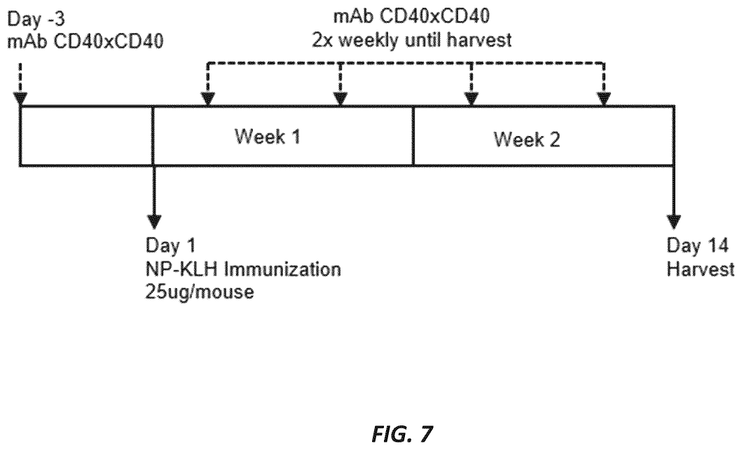

To determine the impact of CD40 blockade on an antigen specific antibody response, a widely used T-dependent immunization model, the NP-KLH immunization model, was utilized in mice homozygous for human CD40 in the place of mouse CD40. Mice were immunized with 25 μg of NP-KLH by subcutaneous injection on the flank with 100 μL of a 1:1 mixture of NP-KLH and alum emulsified by shaking for 30 minutes. For the group receiving alum alone, 100 μL of a 1:1 mixture of PBS and alum was administered. As shown in FIG. 7, anti-CD40×CD40 bispecific antibody (REGN16334, REGN16335, REGN16431, or REGN16432) or isotype control (REGN4439 or REGN4460) was administered to mice 3 days before immunization, and twice per week following immunization for a total of two weeks at a dose of 1 mg/kg. Following immunization and the antibody treatment protocol, the mice were sacrificed, and blood and inguinal lymph nodes were collected. The blood was collected from all groups of mice by cardiac puncture and transferred into BD microtainer tubes (cat #365967) for serum isolation.

For tissue processing, lymph nodes were mashed on a 74-micron cell strainer in 2 mL RPMI media+10% FBS using the back end of a 3 mL syringe, and single cell suspensions were filtered through Millipore plate filter (100 μm) into a 2 mL deep well plate. Cells were centrifuged at 400 g for 4 minutes and resuspended in in 200 μL of PBS. Cells were transferred to a 96 well u-bottom plate, centrifuged at 400 g for 4 minutes and stained with a live/dead cell marker for 15 minutes at room temperature. Cells were washed and incubated in Fc block for 15 minutes at 4° C. followed by antibody staining with antibody mixes as shown in Table 32 for 30 minutes at 4° C. After staining, the cells were washed twice with MACS buffer, fixed with BD Cytofix (cat #554655) diluted 1:4 in PBS for 15 minutes, then resuspended in MACS buffer and stored at 4° C. On the day of acquisition, the cells were washed with BD Perm/wash, incubated in BD Perm/wash buffer (cat #554723) for 20 minutes and stained with intracellular antibodies (Table 32) for 30 minutes. Cells were washed twice and fixed with BD Cytofix, then resuspended in MACS buffer. The cells were then acquired in an FACSymphony A5 instrument and analyzed using OMIQ software. NP+ germinal center B cells were identified as Live→Dump- (dump includes TCRb, CD200R3, Ly6G, CD49b and CD11b)→non-marginal zone B cells (CD1d−)→CD19+1B220+→CD38-IgD−→GC positive (GL7+CD95+)→NP+. Statistical significance was determined by the Shaprio-Wilk test to assess normality and the Kruskal-Wallis test with Dunn's post-hoc multi-comparison test in GraphPad Prism.

TABLE 32
Flow Cytometry Panel.

| | Name/Antigen | Conjugate | Laser | Reactivity | Host | Clone | Isotype | Supplier |
|---|---|---|---|---|---|---|---|---|
| Pre-Stain | Live/Dead | Blue | UV 355 nm | N/A | N/A | N/A | N/A | Invitrogen |
| | Fc Block | None | N/A | Human | Rat | 2.4G2 | N/A | TONGO |
| | (CD16/32) | | | | | | | biosciences |

| | Antigen | Conjugate | Laser | Reactivity | Host | Clone | Isotype | Supplier |
|---|---|---|---|---|---|---|---|---|
| Extracellular | CD38 | BUV395 | UV 355 nm | Mouse/Human | Rat | 90/CD38 | IgG2a, k | BD |
| | CD138 | BV711 | Violet 405 nm | Mouse | Rat | 281-2 | IgG2a, κ | BD |
| | NP | PE | Yellow-Green | | | | | Biosearch |
| | | | 561 nm | | | | | Tech |
| | CD95 | BV421 | Violet 405 nm | Mouse | Hamster | Jo2 | IgG2, λ | BD |
| | GL-7 | PerCP- | Blue 488 nm | Mouse/Human | Rat | GL7 | IgM, k | Biolegend |
| | | Cy5.5 | | | | | | |
| | IgD | BV786 | Violet 405 nm | Mouse | Rat | 11-26c.2a | IgG2a, k | BD |
| | IgA | FITC | Blue 488 nm | Mouse | Rat | C10-3 | IgG1, κ | BD |
| | IgG1 | BV510 | Violet 405 nm | Mouse | Rat | A85-1 | IgG1, κ | BD |
| | CD19 | BUV737 | UV 355 nm | Mouse | Rat | 1D3 | IgG2a, κ | BD |
| | B220 | PE-Cy7 | Yellow-Green | Mouse | Rat | RA3-6B2 | IgG2a, κ | BD |
| | | | 561 nm | | | | | |
| | CD98 | BV605 | Violet 405 nm | Mouse | Hamster | H202-141 | IgG2a, κ | BD |
| | CD1d | BUV563 | UV 355 nm | Mouse | Rat | WTH2 | IgG2a, κ | BD |
| | Dump | | | | | | | BD |
| | TCRβ | APC | Red 640 nm | Mouse | Hamster | H57-597 | IgG₂, λ1 | BD |
| | CD200R3 | APC | Red 640 nm | Mouse | Rat | Ba13 | | Biolegend |
| | Ly6G | APC | Red 640 nm | Mouse | Rat | 1A8-Ly6G | IgG2a, κ | eBioscience |
| | CD49b | APC | Red 640 nm | Mouse | Rat | DX5 | IgM, κ | Biolegend |
| | CD11b | APC | Red 640 nm | Mouse | Rat | M1/70 | IgG2b, k | Biolegend |
| Intracellular | Light Chain k | BV650 | Violet 405 nm | Mouse | Rat | 187.1 | IgG1, k | BD |
| | Light Chain 1 | BV650 | Violet 405 nm | Mouse | Rat | R26-46 | IgG2a, κ | BD |
| | IgA | FITC | Blue 488 nm | Mouse | Rat | C10-3 | IgG1, κ | BD |
| | IgG1 | BV510 | Violet 405 nm | Mouse | Rat | A85-1 | IgG1, k | BD |
| | NP | PE | Yellow-Green | | | | | Biosearch |
| | | | 561 nm | | | | | Tech |

### Experiment #1—Methods and Results

In one experiment, levels of NP-specific IgG1 were qualified in terminal serum by ELISA. NP-2-B3SA was diluted into 4 μg/mL in PBS and 384 well plates were coated with 25 μL/well of solution overnight at 4° C. Plates were washed 4× with wash buffer and blocked with 50 μL/well of 0.5% BSA in PBS for 1 hour at room temperature. Plates were washed 4× with wash buffer and the mouse serum was diluted 1:100 for group A and 1:10,000 for groups B-G. Serum was further serially diluted 3-fold using 0.5% BSA in PBS 8 times and added to plates at 12.5 μL/well. Diluted serum was incubated on plates for 1 hour at room temperature followed by 4× wash. For detection, 25 μL of rat anti-mouse IgG1 HRP conjugated antibody diluted 1:1000 in 0.5% BSA in PBS was added to plates for 1 hour at room temperature. Plates were washed 7× and developed by adding 25 μL of using OptEIA™ TMB Substrate solution. After 20 minutes the reaction was stopped by adding 25 μL 2N sulfuric acid. Absorbance at 450 nm (OD450) was measured on a Molecular Devices SpectraMax M5 plate reader. Relative levels of circulating NP-specific IgG1 in serum were represented as titer units which were defined as dilution factor required to achieve an OD450 reading that was equal to two times background OD450. Graphical analyses were performed using GraphPad Prism software (version 7.0). Statistical significance was determined by the Shaprio-Wilk test to assess normality and the Kruskal-Wallis test with Dunn's post-hoc multi-comparison test in GraphPad Prism.

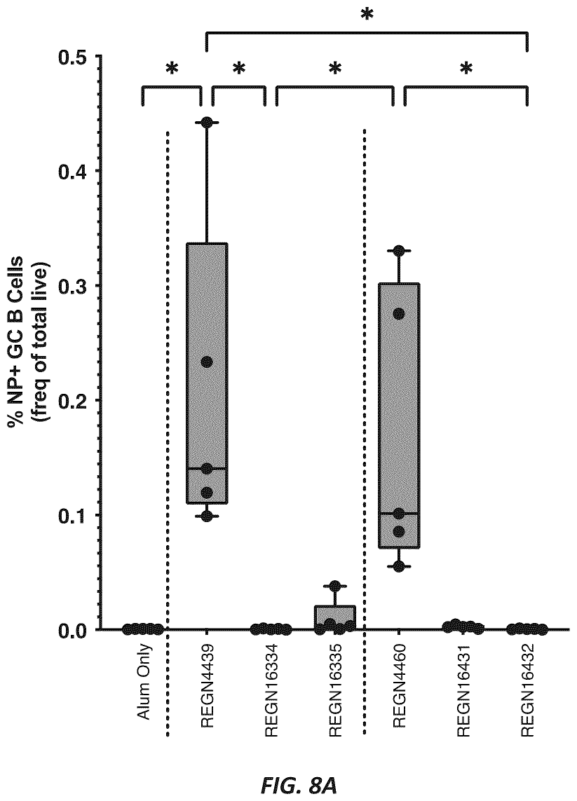

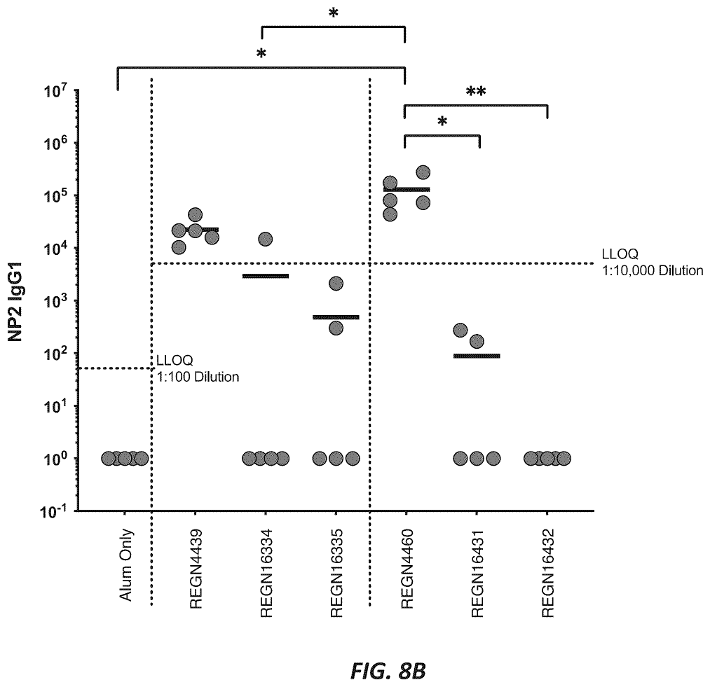

In this NP-KLH immunization model, prophylactic treatment with anti-CD40×CD40 bispecific antibody blocked antigen specific germinal center B cell formation in the draining lymph node as quantified by flow cytometry. All of the anti-CD40×CD40 bispecific antibodies reduced the frequency of NP-specific germinal center B cells at 1 mg/kg compared to their relevant isotype controls (FIG. 8A and Table 33). Prophylactic treatment with anti-CD40×CD40 bispecific antibody at 1 mg/kg also blocked the generation of high-affinity NP-IgG1 antibody responses as measured by NP-specific IgG1 titers in serum, with most samples falling below the lower limit of quantification for the assay (FIG. 8B and Table 34).

TABLE 33
Effect of anti-CD40xCD40 bispecific antibodies on frequency of NP+
germinal center B cells.

| | | | Mean % NP+ | Mean rank |
|---|---|---|---|---|
| | | | Germinal | difference |
| | | | center B | compared |
| | | Antibody | cells (Freq. of | to isotype |
| Group | Treatment | Dose | Live) ± SD | control |
| A | Alum (no mAb) | No mAb | 0.0006019 ± 0.0002945 | N/A |
| B | REGN4439 | 1 mg/kg | 0.2066 ± 0.1413 | N/A |
| | (isotype) | | | |
| C | REGN16334 | 1 mg/kg | 0.0005531 ± 0.0005146 | 21.9 |
| | | | | (* p = 0.0153) |
| D | REGN16335 | 1 mg/kg | 0.009392 ± 0.01611 | 14.4 (ns) |
| E | REGN4460 | 1 mg/kg | 0.1693 ± 0.1243 | N/A |
| | (isotype) | | | |
| F | REGN16431 | 1 mg/kg | 0.002636 ± 0.00141 | 10.6 (ns) |
| G | REGN16432 | 1 mg/kg | 0.0005721 ± 0.0004048 | 20.3 |
| | | | | (* p = 0.0364) |

SD = standard deviation; N/A = not available; ns = not statistically significant.
* p < 0.05.

TABLE 34
Effect of anti-CD40xCD40 bispecific antibodies on frequency of NP
IgG1 titers in mouse serum.

| | | | | Mean rank |
|---|---|---|---|---|
| | | | | difference |
| | | | | compared |
| | | Antibody | Mean NP | to isotype |
| Group | Treatment | Dose | Titers ± SD | control |
| A | Alum (no mAb) | No mAb | *no values detected | N/A |
| B | REGN4439 (isotype) | 1 mg/kg | 22349 ± 12298 | N/A |
| C | REGN16334 | 1 mg/kg | 2942 ± 6576 | 14.2 (ns) |
| D | REGN16335 | 1 mg/kg | 485.3 ± 925.3 | 12.1 (ns) |
| E | REGN4460 (isotype) | 1 mg/kg | 129249 ± 95137 | N/A |
| F | REGN16431 | 1 mg/kg | 88.9 ± 126.0 | 18.1 |
| | | | | (* p = 0.0412) |
| G | REGN16432 | 1 mg/kg | *no values detected | 22.5 |
| | | | | (** p = 0.0025) |

SD = standard deviation; N/A = not available; ns = not statistically significant.
* p < 0.05; ** p < 0.005.

### Experiment #2—Methods and Results

In another experiment, levels of NP-specific IgG1 were qualified in terminal serum by ELISA. NP-2-BSA was diluted to 4 μg/mL in PBS and 384 well plates were coated with 25 μL/well of solution overnight at 4° C. Plates were washed 5× with wash buffer and blocked with 50 μL/well of 0.5% BSA in PBS for 1 hour at room temperature. Plates were washed 5× with wash buffer and the mouse serum was diluted 1:100 for group A-B and 1:10,000 for groups C-L. Serum was further serially diluted 3-fold using 0.5% BSA in PBS 8 times and added to plates at 12.5 μL/well. Diluted serum was incubated on plates for 1 hour at room temperature followed by 4× wash. For detection, 25 μL of rat anti-mouse IgG1 HRP conjugated antibody diluted 1:1,000 in 0.5% BSA in PBS was added to plates for 1 hour at room temperature. Plates were washed 7× and developed by adding 25 μL of using OptEIA™ TMB Substrate solution. After 20 minutes the reaction was stopped by adding 25 μL 2N sulfuric acid. Absorbance at 450 nm (OD450) was measured on a Molecular Devices SpectraMax M5 plate reader. Relative levels of circulating NP-specific IgG1 in serum were represented as titer units which were defined as dilution factor required to achieve an OD450 reading that was equal to two times background OD450. Data analysis was performed using GraphPad Prism. Statistical significance was determined by the Shaprio-Wilk test to assess normality and the Kruskal-Wallis test with Dunn's post-hoc multi-comparison test in GraphPad Prism.

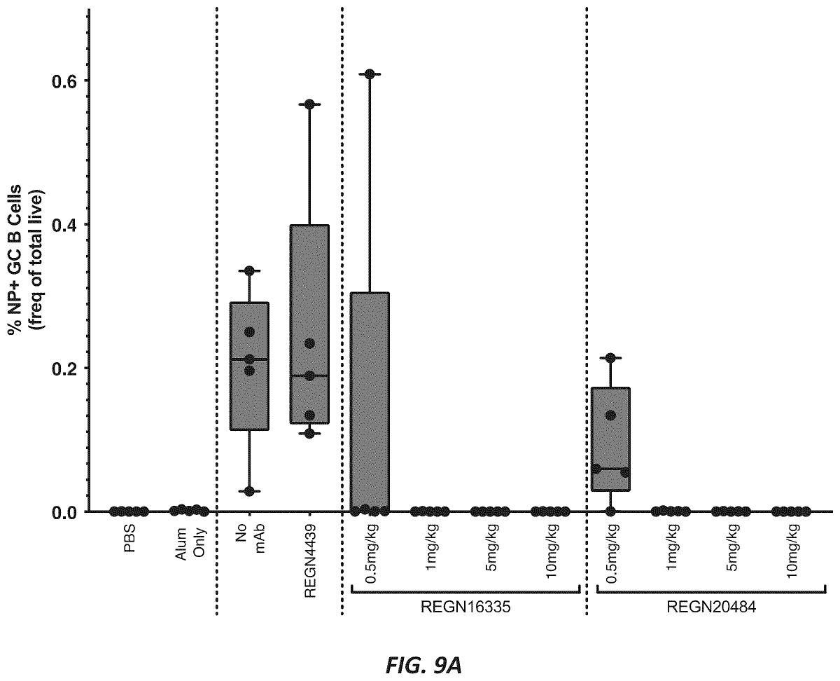

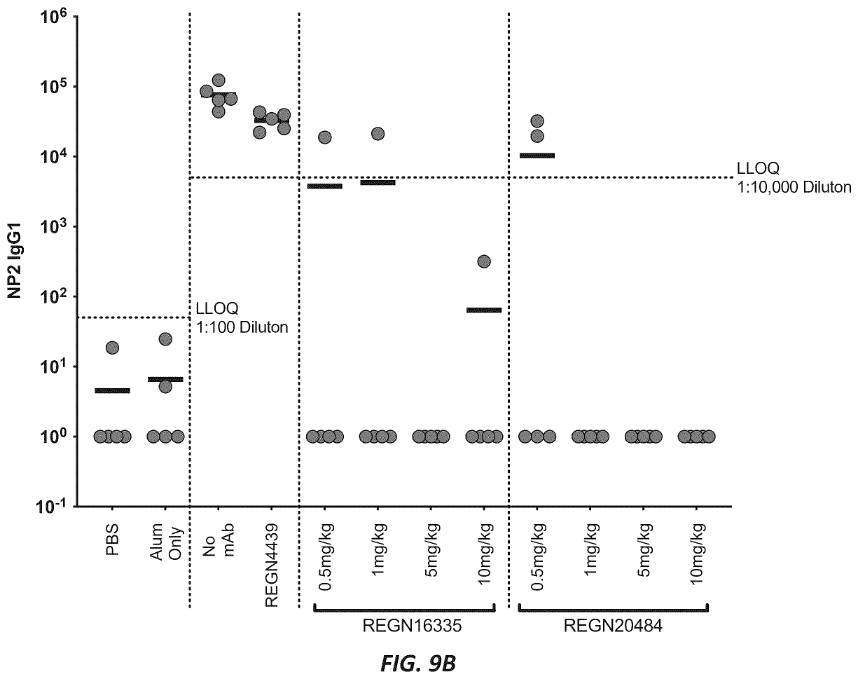

In this experiment, prophylactic treatment with anti-CD40×CD40 bispecific antibody blocked antigen specific germinal center B cell formation in the draining lymph node as quantified by flow cytometry. All of the tested anti-CD40×CD40 bispecific antibodies reduced the frequency of NP-specific germinal center B cells at 1 mg/kg and higher compared to isotype control (FIG. 9A and Table 35). Prophylactic treatment with anti-CD40×CD40 bispecific antibody at 1 mg/kg also blocked the generation of high-affinity NP-IgG1 antibody responses as measured by NP-specific IgG1 titers in serum, with most samples falling below the lower limit of quantification for the assay (FIG. 9B and Table 36).

TABLE 35
Effect of anti-CD40xCD40 bispecific antibodies on frequency of NP+
germinal center B cells.

| | | | | Mean rank |
|---|---|---|---|---|
| | | | | difference |
| | | | Mean % NP+ Germinal | compared |
| | Antibody | | center B cells (Freq. of | to isotype |
| Treatment | Dose | Group | Live) +/− SD | control |
| PBS Only | No mAb | A | 0.00007863 ± 0.0001758 | N/A |
| Alum | No mAb | B | 0.00168 ± 0.001403 | N/A |
| (no mAb) | | | | |
| NP-KLH | No mAb | C | 0.2042 ± 0.1121 | N/A |
| Only | | | | |
| REGN4439 | 10 mg/kg | D | 0.2466 ± 0.1856 | N/A |
| (Isotype | | | | |
| control) | | | | |
| REGN16335 | 0.5 mg/kg | E | 0.1228 ± 0.2718 | 11.9 (ns) |
| | 1 mg/kg | F | 0.0002173 ± 0.0003573 | 32.9 (ns) |
| | 5 mg/kg | G | 0.00006046 ± 0.0001352 | 40.1 |
| | | | | (* p = 0.0154) |
| | 10 mg/kg | H | 0.0001546 ± 0.000254 | 35.8 (ns) |
| REGN20484 | 0.5 mg/kg | I | 0.09259 ± 0.08283 | 7 (ns) |
| | 1 mg/kg | J | 0.000671 ± 0.0006068 | 21.1 (ns) |
| | 5 mg/kg | K | 0.0002724 ± 0.0003113 | 31.1 (ns) |
| | 10 mg/kg | L | 0.00005632 ± 0.00008618 | 38.2 |
| | | | | (* p = 0.03) |

SD = standard deviation; N/A = not available; ns = not statistically significant.
* p < 0.05.

TABLE 36
Effect of anti-CD40xCD40 bispecific antibodies on frequency
of NP IgG1 titers in mouse serum.

| | | | | Mean rank |
|---|---|---|---|---|
| | | | | difference |
| | Antibody | | Mean NP | compared to |
| Treatment | Dose | Group | Titers +/− SD | isotype control |
| PBS Only | No mAb | A | 4.513 ± 7.855 | N/A |
| Alum (no mAb) | No mAb | B | 6.582 ± 10.3 | N/A |
| NP-KLH Only | No mAb | C | 76584 ± 29915 | N/A |
| REGN4439 | 10 mg/kg | D | 32900 ± 9061 | N/A |
| (Isotype | | | | |
| control) | | | | |
| REGN16335 | 0.5 mg/kg | E | 3762 ± 8409 | 26 (ns) |
| | 1 mg/kg | F | 4238 ± 9474 | 25.6 (ns) |
| | 5 mg/kg | G | *no values detected | 31.1 (* p = 0.0339) |
| | 10 mg/kg | H | 64.18 ± 141.3 | 26.2 (ns) |
| REGN20484 | 0.5 mg/kg | I | 10352 ± 14860 | 19.7 (ns) |
| | 1 mg/kg | J | *no values detected | 31.1 (* p = 0.0339) |
| | 5 mg/kg | K | *no values detected | 31.1 (* p = 0.0339) |
| | 10 mg/kg | L | *no values detected | 31.1 (* p = 0.0339) |

SD = standard deviation; N/A = not available; ns = not statistically significant.
*p < 0.05.

### Example 10. Impact of Prophylactic Dosing with Anti-CD40×CD40 Bispecific Antibodies in a Mouse Model of Experimental Autoimmune Encephalomyelitis (EAE)

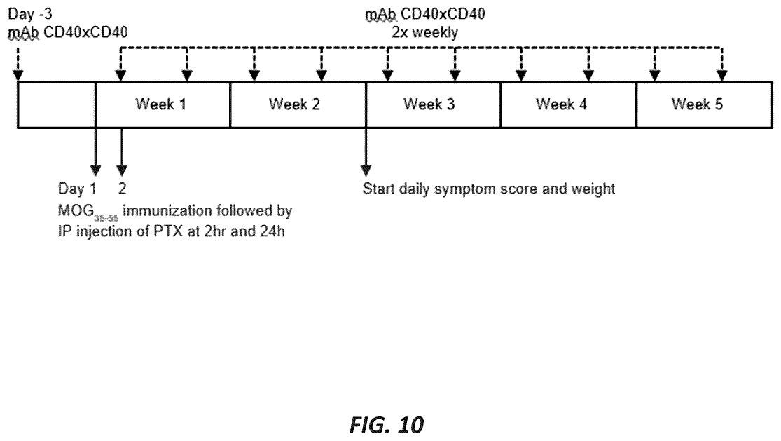

To determine the impact of CD40 blockade in an autoimmune disease, a model of experimental autoimmune encephalomyelitis (EAE) model was utilized in mice where the entire mouse CD40 gene was replaced by full-length human CD40 (CD40^{hu/hu} mice). Mice were immunized by subcutaneous injection of 0.1 mL MOG35-55 in emulsion with complete Freund's adjuvant on the upper back and lower back (0.2 mL/mouse total). Two hours following immunization, mice were administered 150 ng pertussis toxin (PTX) intraperitoneally at 0.1 mL/dose. The administration of PTX was repeated 24 hours later. Anti-CD40×CD40 bispecific antibodies or isotype controls were administered to mice 3 days before immunization at 25 mg/kg, and twice a week following immunization for a total of five weeks. See, FIG. 10. Mice were monitored and weighed twice a week for the first two weeks following immunization and daily for weeks 3-5. Mice were assigned a score following the scoring guidelines outlined in Table 37. Mice were euthanized after receiving a symptom score of 4 for two consecutive days or upon a score of 4.5 or 5 or when they had dropped 30% of their starting body weight.

TABLE 37
Mouse EAE Scoring Guidelines.

| Score | Description |
|---|---|
| 0 | No symptoms |
| 0.5 | Tip of tail is limp. When picked up by base of tail, |
| | the tail has tension except for the tip. Muscle straining |
| | is felt in the tail, while the tail continues to move. |
| 1 | Wholly limp tail. When picked up by base of tail, |
| | instead of being erect, the whole tail drapes |
| | over finger. Hind legs are usually spread apart. |
| | No signs of tail movement are observed. |
| 1.5 | Limp tail and hind leg inhibition. When picked |
| | up by base of tail, the whole tail drapes over |
| | finger. When the mouse is dropped on a wire |
| | rack, at least one hind leg falls through |
| | consistently. Walking is very slightly wobbly. |
| 2 | Limp tail and weakness of hind legs. When |
| | picked up by base of tail, the legs are not spread |
| | apart, but held closer together. When the mouse |
| | is observed walking, it has a clearly apparent |
| | wobbly walk. One foot may have toes dragging, |
| | but the other leg has no apparent inhibitions of |
| | movement. -OR- Mouse appears to be at score 0.0, |
| | but there are obvious signs of head tilting |
| | when the walk is observed. The balance is poor. |
| 2.5 | Limp tail and dragging of hind legs. Both |
| | hind legs have some movement, but both are |
| | dragging at the feet (mouse trips on hind feet). |
| | -OR- No movement in one leg/completely |
| | dragging one leg, but movement in the other leg. |
| | -OR- EAE severity appears mild when picked |
| | up (as score 0.0-1.5), but there is a strong head |
| | tilt that causes the mouse to occasionally fall |
| | over. |
| 3 | Limp tail and complete paralysis of hind legs |
| | (most common). -OR- Limp tail and almost |
| | complete paralysis of hind legs. One or both hind |
| | legs are able to paddle, but neither hind leg is |
| | able to move forward of the hind hip. -OR- |
| | Limp tail with paralysis of one front and one |
| | hind leg. -OR- ALL of: Severe head tilting, |
| | Walking only along the edges of the cage, |
| | Pushing against the cage wall, Spinning |
| | when picked up by base of tail. |
| 3.5 | Limp tail and complete paralysis of hind legs. |
| | In addition to: Mouse is moving around the |
| | cage, but when placed on its side, is unable to |
| | right itself. Hind legs are together on one side |
| | of body. -OR- Mouse is moving around the |
| | cage, but the hind quarters are flat like a |
| | pancake, giving the appearance of a hump in |
| | the front quarters of the mouse. |
| 4 | Limp tail, complete hind leg and partial front |
| | leg paralysis. Mouse is minimally moving |
| | around the cage but appears alert and feeding. |
| | Mouse will be euthanized if it scores 4.0 for 2 |
| | days. |
| 4.5 | Complete hind and partial front leg paralysis, |
| | no movement around the cage. Mouse is not |
| | alert. Mouse has minimal movement in the |
| | front legs. The mouse barely responds to contact. |
| | Mice will be euthanized if this score is registered. |
| | When the mouse is euthanized because of |
| | severe paralysis, a score of 5.0 is entered for that |
| | mouse for the rest of the experiment. |
| 5 | Mouse is spontaneously rolling in the cage |
| | -OR- Mouse is found dead due to paralysis. |
| | -OR- Mouse is euthanized due to severe paralysis. |

### Experiment #1—Results

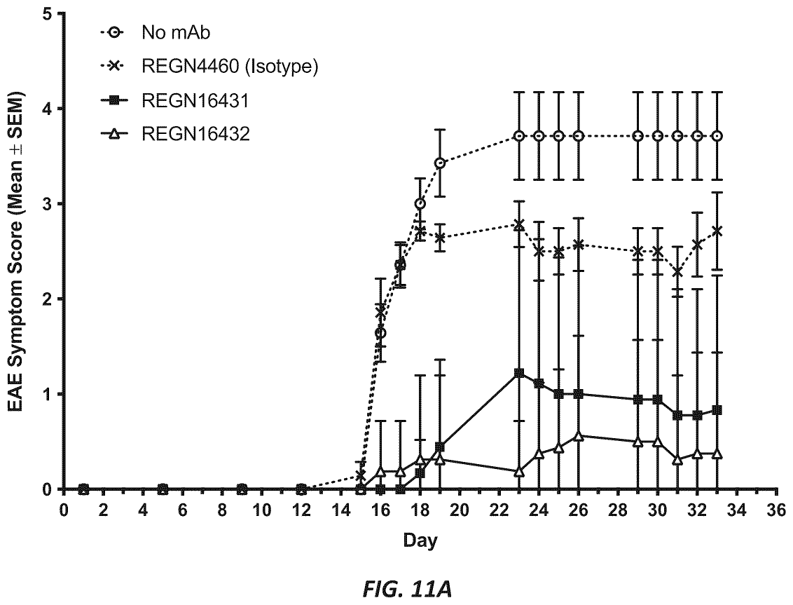

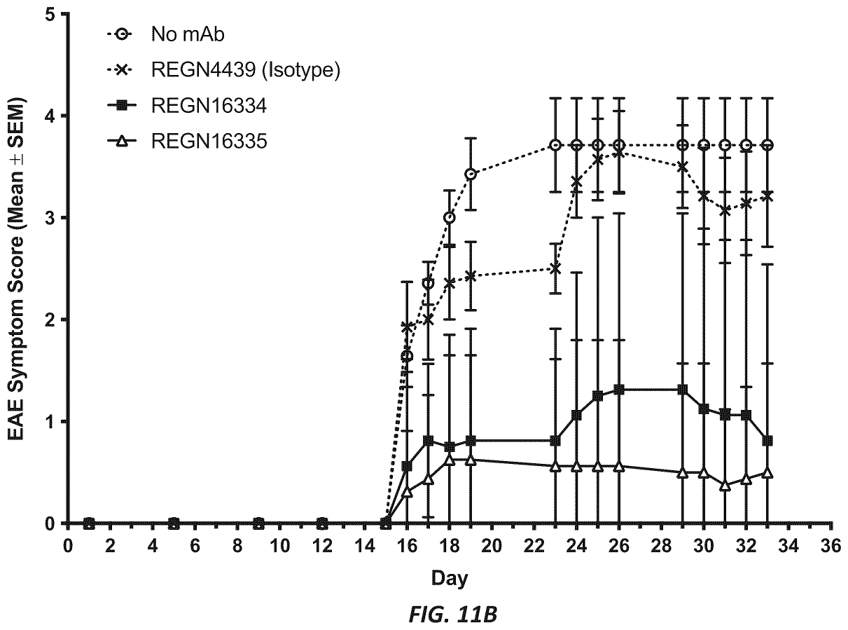

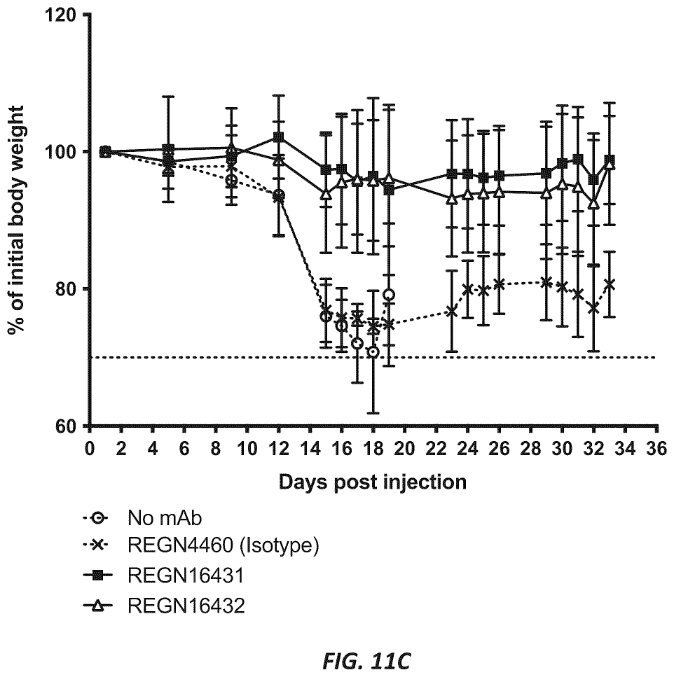

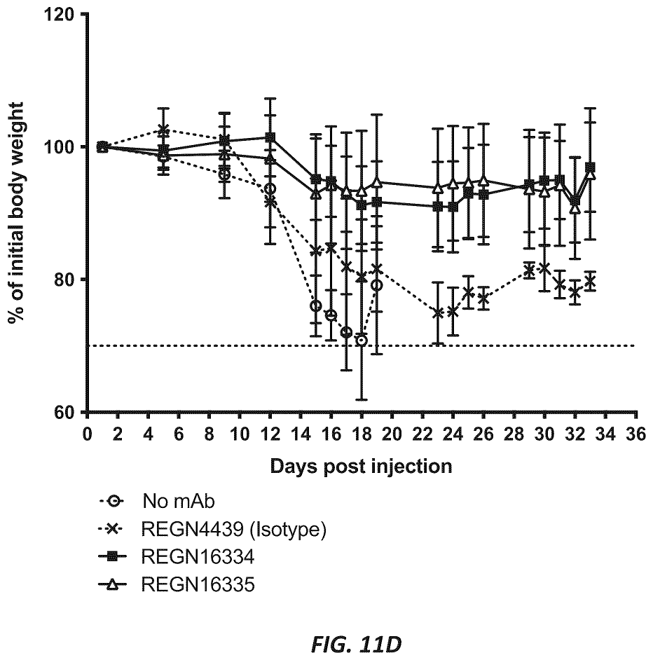

In one experiment using a model of experimental autoimmune encephalomyelitis (EAE), prophylactic blockade of CD40 by anti-CD40×CD40 bispecific antibodies REGN16334, REGN16335, REGN16431, and REGN16432 reduced the severity of EAE disease symptom score relative to no antibody or isotype control treated groups (FIGS. 11A-11B and Table 38). In addition to reduction of symptom severity, all CD40 blocking antibodies reduced the percentage of mouse weight loss when compared to no antibody or isotype control treated groups (FIGS. 11C-11D and Table 39). The frequency of mice with disease score development was also reduced in groups treated with CD40 blocking antibodies (Table 40).

TABLE 38
Mean EAE Symptom Score.

| | Terminal Mean | Total Mean |
|---|---|---|
| Treatment | EAE score ± SD | EAE score ± SD |
| Alum (no mAb) | 3.71 ± 1.22 | 2.44 ± 1.83 |
| REGN4439 (Isotype control) | 3.21 ± 1.32 | 2.11 ± 1.65 |
| REGN16334 | 0.81 ± 1.73 | 0.71 ± 1.27 |
| REGN16335 | 0.50 ± 1.07 | 0.36 ± 0.85 |
| REGN4460 (Isotype control) | 2.71 ± 1.07 | 1.81 ± 1.27 |
| REGN16431 | 0.83 ± 1.41 | 0.51 ± 1.10 |
| REGN16432 | 0.38 ± 1.06 | 0.26 ± 0.73 |

TABLE 39
Mean Weight Loss.

| | Terminal % of initial | Total % of initial |
|---|---|---|
| Treatment | body weight ± SD | body weight ± SD |
| Alum (no mAb) | 69.99 ± 8.18 | 86.5 ± 12.52 |
| REGN4439 (Isotype control) | 76.4 ± 5.72 | 85.68 ± 10.93 |
| REGN16334 | 94.8 ± 8.65 | 94.94 ± 6.56 |
| REGN16335 | 95.89 ± 9.89 | 95 ± 7.93 |
| REGN4460 (Isotype control) | 77.28 ± 6.96 | 83.06 ± 9.80 |
| REGN16431 | 95.37 ± 11.89 | 97.6 ± 7.41 |
| REGN16432 | 98.21 ± 8.91 | 95.95 ± 8.35 |

TABLE 40
Number of Mice with Development of EAE Symptoms.

| Antibody (2x weekly) | No. of mice with EAE symptom score |
|---|---|
| No antibody | 7 out of 7 |
| REGN4439 (Isotype control) | 7 out of 7 |
| REGN16334 | 7 out of 8 |
| REGN16335 | 3 out of 8 |
| REGN4460 (Isotype control) | 7 out of 7 |
| REGN16431 | 4 out of 8 |
| REGN16432 | 2 out of 8 |

### Experiment #2—Results

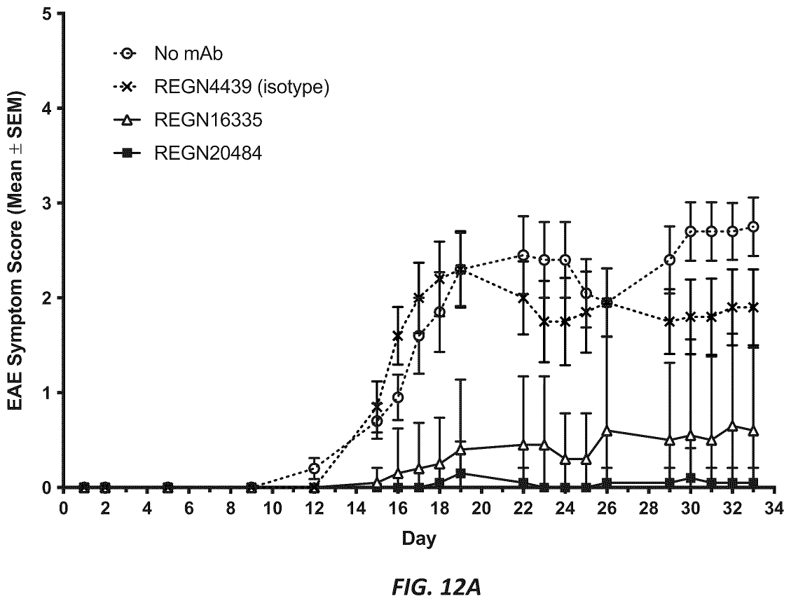

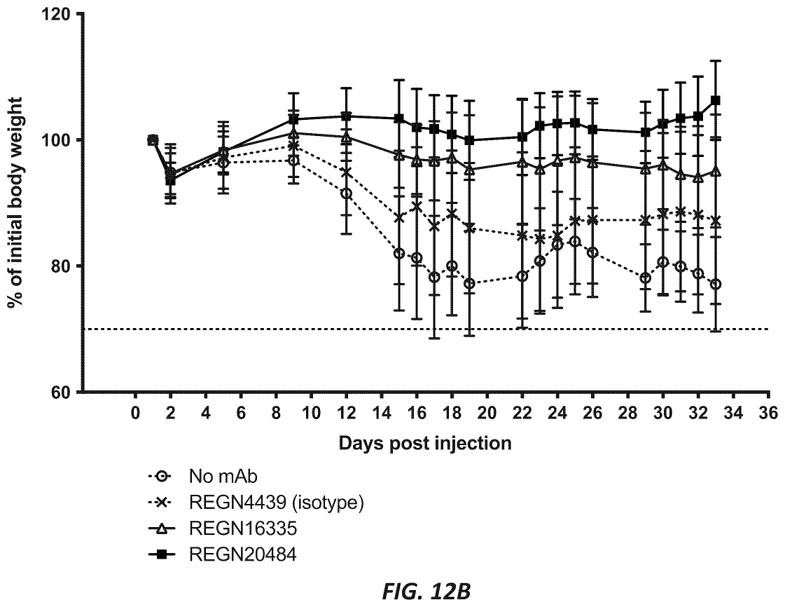

In another experiment, prophylactic blockade of CD40 by anti-CD40×CD40 bispecific antibodies REGN16335 or REGN20484 reduced the severity of EAE disease symptom score relative to no antibody or isotype control treated groups (FIG. 12A and Table 41). In addition to reduction of symptom severity, all CD40 blocking antibodies reduced the percentage of mouse weight loss when compared to no antibody or isotype control treated groups (FIG. 12B and Table 42). The frequency of mice with disease score development was also reduced in groups treated with CD40 blocking antibodies (Table 43).

TABLE 41
Mean EAE Symptom Score.

| | Terminal Mean EAE | Total Mean EAE |
|---|---|---|
| Treatment | score ± SD | score ± SD |
| Alum (no mAb) | 2.75 ± 0.98 | 1.61 ± 1.39 |
| REGN4439 (Isotype control) | 1.9 ± 1.26 | 1.37 ± 1.31 |
| REGN16335 | 0.6 ± 0.88 | 0.30 ± 0.64 |
| REGN20484 | 0.05 ± 0.16 | 0.03 ± 0.14 |

TABLE 42
Mean Weight Loss.

| | Terminal % of initial | Total % of initial |
|---|---|---|
| | body weight ± | body weight ± |
| Treatment | SD | SD |
| Alum (no mAb) | 69.99 ± 8.18 | 86.5 ± 12.52 |
| REGN4439 (Isotype control) | 76.4 ± 5.72 | 85.68 ± 10.93 |
| REGN16334 | 94.8 ± 8.65 | 94.94 ± 6.56 |
| REGN16335 | 95.89 ± 9.89 | 95 ± 7.93 |
| REGN4460 (Isotype control) | 77.28 ± 6.96 | 83.06 ± 9.80 |
| REGN16431 | 95.37 ± 11.89 | 97.6 ± 7.41 |
| REGN16432 | 98.21 ± 8.91 | 95.95 ± 8.35 |

TABLE 43
Number of Mice with Development of EAE Symptoms.

| Group | Treatment | No. of mice with EAE symptom score |
|---|---|---|
| A | Alum (no mAb) | 10 out of 10 |
| B | REGN4439 (Isotype control) | 10 out of 10 |
| C | REGN16335 | 5 out of 10 |
| D | REGN20484 | 3 out of 10 |

### Example 11. CD40L Blockade Reduces the Antibody Response to AAV, but Enables Only Partial Transduction Upon Vector Re-Administration

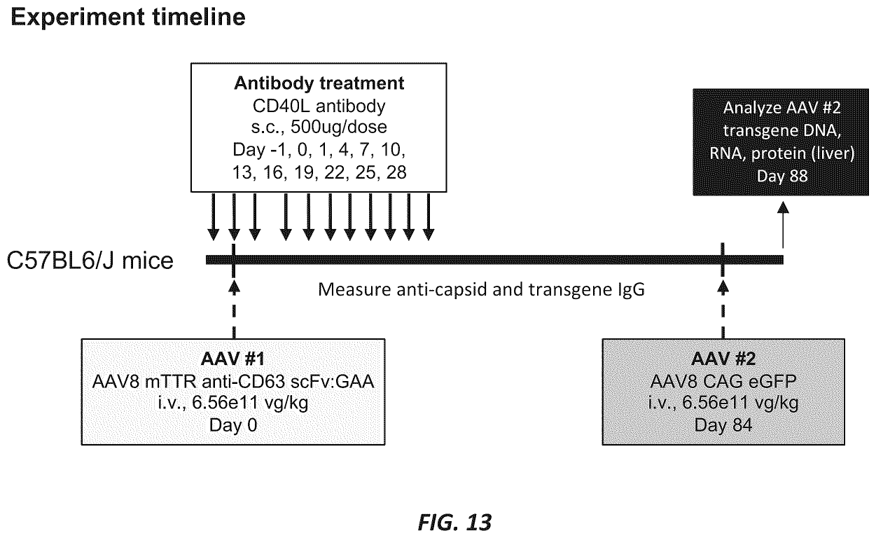

To test the impact of CD40L blockade to enable vector re-administration, a study was conducted in mice, outlined in FIG. 13. C57BL/6 mice (n=4-5 mice per group) were dosed prophylactically two times with 500 g of anti-mouse CD40L blocking antibody (InVivoMAb anti-mouse CD40L (CD154), clone MR-1, bioxcell.com/invivomab-anti-mouse-cd40l-cd154-be0017-1, BioXcell, Lebanon, NH), subcutaneously under the skin on the back. Control mice were left untreated. Anti-CD40L antibody-injected and control mice were then dosed with a recombinant AAV8 vector (“AAV #1,” encoding human lysosomal acid alpha glucosidase with an N-terminal fusion to a human single-chain variable fragment specific to human CD63; hereafter, “anti-CD63 scFv:hGAA”) intravenously via retroorbital injection at 6.56e11 vector genomes per kilogram body weight (vg/kg). After AAV #1 injection, the treatment group continued to receive subcutaneous administration of 500 g of anti-CD40L antibody every three days, until day 28 of the study. Mice were bled at defined intervals for serum anti-AAV antibody analyses.

To assess whether CD40L-mediated blockade impacted the development of antibody responses to AAV, serum levels of anti-AAV capsid IgG were quantified at defined timepoints by ELISA. Specifically, 96-well flat-bottom plates were coated with 1e9 vg/well recombinant AAV8 vector in DPBS overnight. The next day, plates were washed and blocked prior to addition of serum samples at serial 3× dilutions, beginning at an initial dilution of 1:300. Serum was incubated overnight at 4° C. The next day, plates were repeatedly washed prior to incubation with an anti-mouse-IgG Fcg Fragment-HRP-conjugated polyclonal secondary antibody (Jackson Immunoresearch, West Grove, PA). Plates were again repeatedly washed prior to development with TMB Substrate solution. After 20 minutes, the reaction was stopped by addition of 2N phosphoric acid. Absorbance at 450 nm (OD450) was measured on a SpectraMax i3 plate reader (Molecular Devices, San Jose, CA). Relative levels of serum anti-AAV8 IgG were determined and plotted as titer values using Prism v.9 software (GraphPad, Boston, MA). Titer was defined as the dilution factor required to achieve an OD450 reading equal to 2-fold higher than background values.

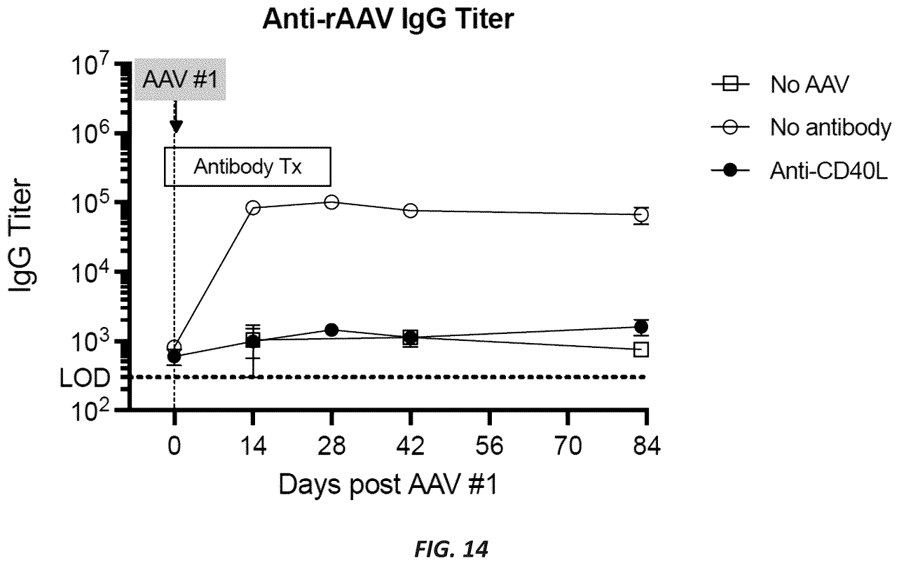

As shown in FIG. 14, antibody-mediated CD40L blockade led to suppression of anti-AAV IgG antibody responses compared to mice that were not treated with antibody, which mounted strong antibody titers.

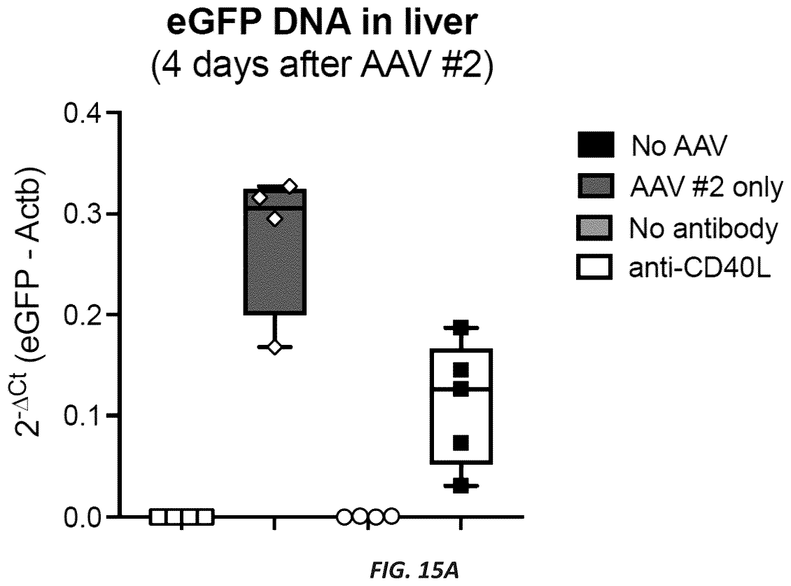

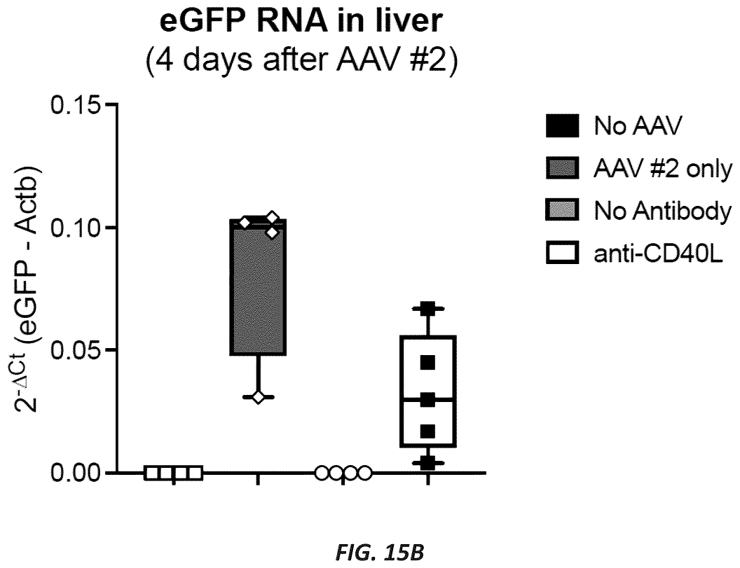

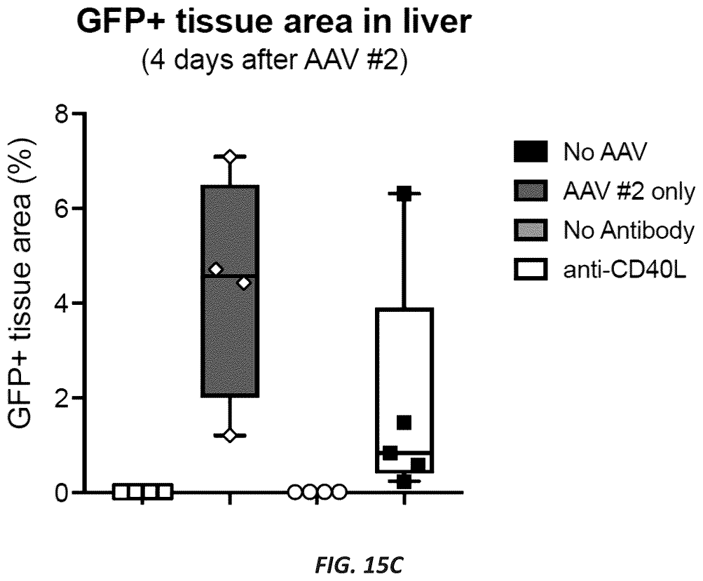

To test whether reduced antibody titers observed in anti-CD40L-treated mice could enable AAV vector re-administration, mice were dosed with a second AAV vector (“AAV #2”) encoding eGFP, 84 days after receiving AAV #1 (see, e.g., FIG. 13). Mice were sacrificed 4 days later for analysis of eGFP transgene DNA and RNA in the liver by quantitative polymerase chain reaction (qPCR). As shown in FIGS. 15A-15B, mice that received anti-CD40L antibody blockade exhibited detectable levels of eGFP transgene DNA and mRNA versus mice that were not treated with antibody. However, these DNA and mRNA levels were on average lower than that of mice that were previously naive to AAV. To further assess eGFP transduction in liver, immunohistochemical analysis of eGFP protein expression was performed on formalin-fixed liver tissue sections, and eGFP+ area was quantified using HALO software (Indica Labs). Consistent with eGFP transgene DNA and mRNA, eGFP protein expression was significantly higher in anti-CD40L antibody treated mice versus mice that received no antibody treatment (FIG. 15C). However, also consistent with eGFP transgene DNA and mRNA findings, the level of eGFP protein expression was again on average lower than that of mice that were previously naive to AAV.

Collectively, these data show that while antibody-mediated blockade of CD40L can suppress antibody responses to AAV vectors, targeting CD40L alone was not sufficient to enable vector re-transduction at levels equivalent to that observed in animals receiving the vector for the first time. These findings are supported by findings in previous reports, which found that CD40L blockade enabled only partial transduction upon vector re-administration, and that treatment with additional immunomodulatory agents was needed to achieve higher levels of transduction (Manning et al. (1998) Hum. Gene Ther. 9(4):477-485).

### Example 12. Impact of Prophylactic Short-Term CD40 Blockade Using Anti-CD40 Antibodies on Development of Anti-AAV Antibody Responses

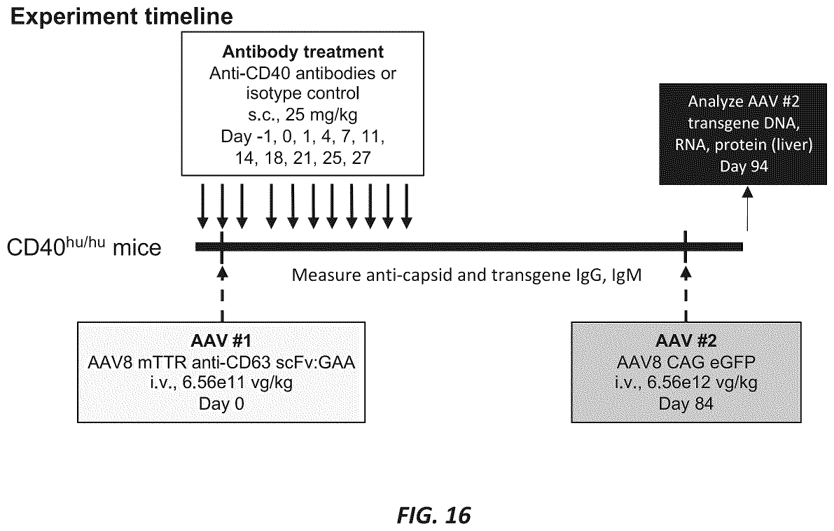

CD40 is constitutively expressed by both B cells and professional antigen presenting cells, whereas expression of its ligand (CD40L) is induced on T cells following activation. Because of this, the inventors reasoned that CD40 blockade may be more effective than CD40L blockade at suppressing anti-AAV antibody responses, since complete CD40 receptor occupancy (and thus more complete blockade of CD40-CD40L interactions that facilitate B cell activation) could be achieved prior to immune activation, rather than concurrent with immune activation. To test this, a study using anti-CD40×CD40 bispecific antibodies in CD40-humanized mice was conducted as outlined in FIG. 16. CD40-humanized mice are mice in which the entire mouse CD40 gene has been replaced with the gene encoding the full-length human CD40 protein; homozygous CD40-humanized mice (CD40hu/hu) are therefore functionally knocked-out for mouse CD40, and instead possess two copies of the human CD40 gene. These mice mount antibody responses at levels similar to wild-type CD40 mice as described herein. As shown in FIG. 16, mice (n=4-5 mice per group) were first administered two prophylactic doses of anti-CD40×CD40 bispecific antagonist antibody (REGN16335 or REGN16334) or equivalent human IgG4 with Fc mutation isotype control (REGN4439), subcutaneously under the neck or back skin at 25 milligrams per kilogram body weight (25 mg/kg). In the same manner, separate groups of mice received a conventional anti-CD40 antagonist monoclonal human IgG1 with Fc mutation (COMP11209, having the CDRs set forth in SEQ ID NOS: 135-140) or human IgG1 isotype control (H1H10126P) at 25 mg/kg. Mice were then administered an AAV8 vector (“AAV #1,” encoding human lysosomal acid alpha glucosidase with an N-terminal fusion to a human single-chain variable fragment specific to human CD63; hereafter, “anti-CD63 scFv:GAA” or “anti-CD63 scFv:hGAA” or “anti-CD63:GAA” or “anti-CD63:hGAA”) intravenously via retroorbital injection at 6.56e11 vg/kg. Mice continued to receive injections of anti-CD40 bispecific or monoclonal antibody, or isotype controls, twice per week for four weeks, and were bled at defined intervals for serum anti-AAV antibody analyses, as described in Example 11. For anti-IgM AAV antibody analysis, an anti-mouse-IgM-chain-specific polyclonal HRP-conjugated antibody was used in place of the anti-IgG HRP-conjugated secondary antibody.

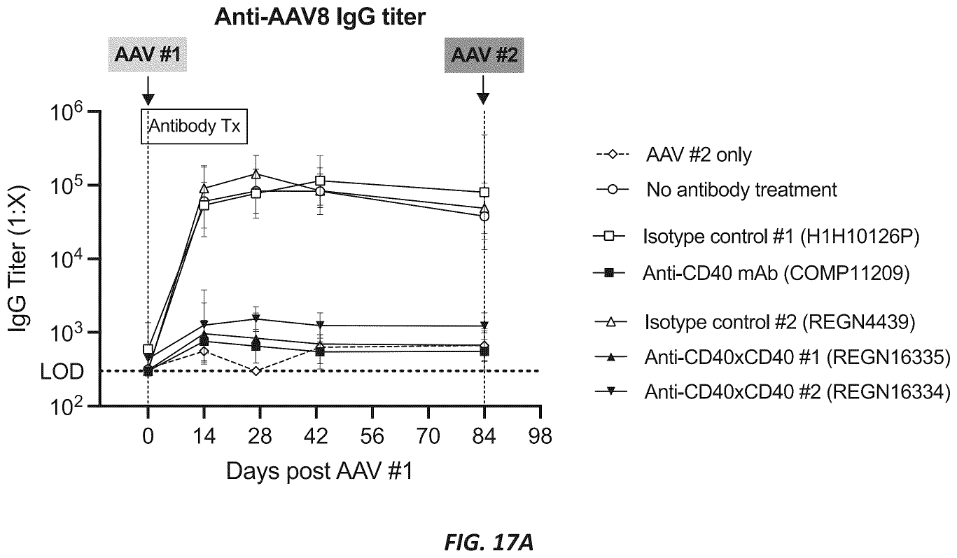

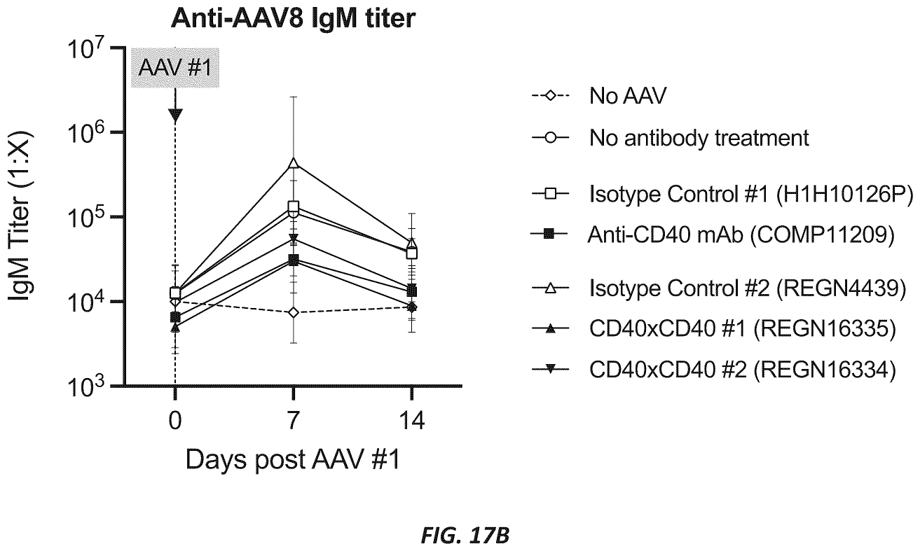

As shown in FIG. 17A, in vivo CD40 blockade with anti-CD40×CD40 bispecific antibodies (REGN16335 or REGN16334) or anti-CD40 mAb (COMP11209) resulted in marked suppression of anti-AAV8 IgG antibody titers to levels similar to AAV-naive animals that did not receive AAV #1 (“AAV #2 only”). By contrast, isotype-control-treated mice, or mice treated with no antibody, showed robust and persistent AAV IgG responses. Evaluation of anti-AAV8 IgM titers also revealed a partial inhibitory effect of anti-CD40 antibodies (FIG. 17B). Thus, these data demonstrate that CD40 blockade with anti-CD40 antibodies can potently suppress de novo IgG responses following AAV vector administration.

### Example 13. Impact of Prophylactic CD40 Blockade on Ability to Systemically Re-Administer AAV Vector

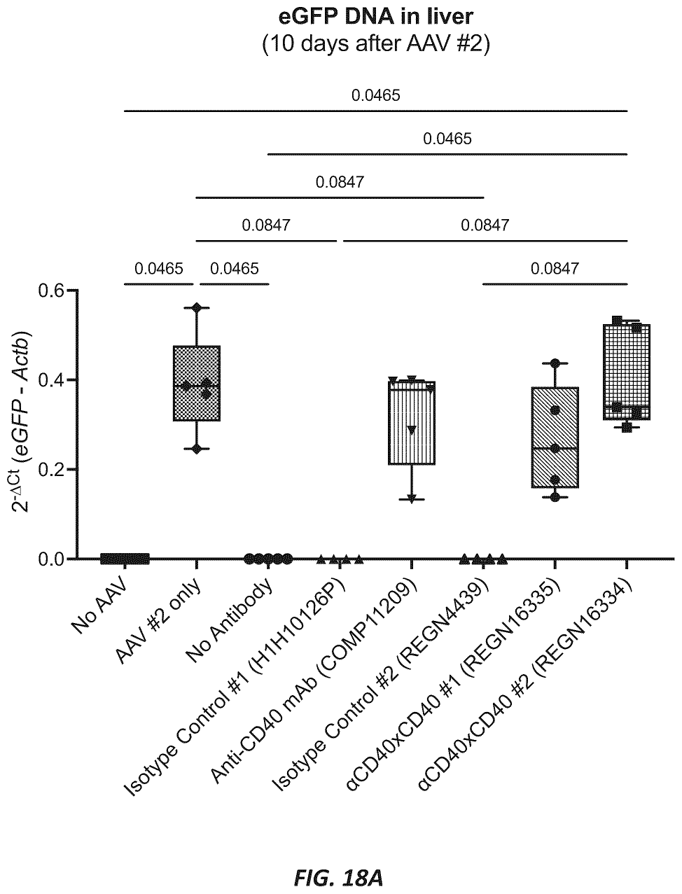

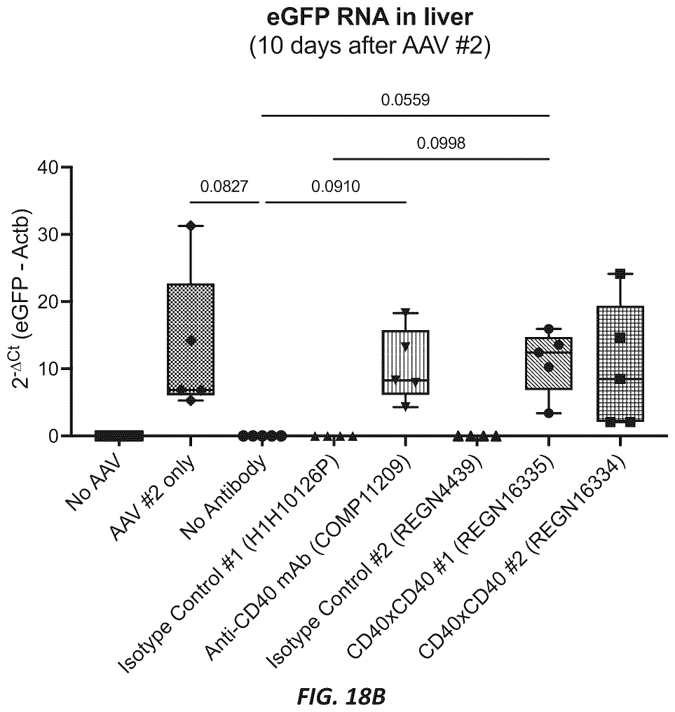

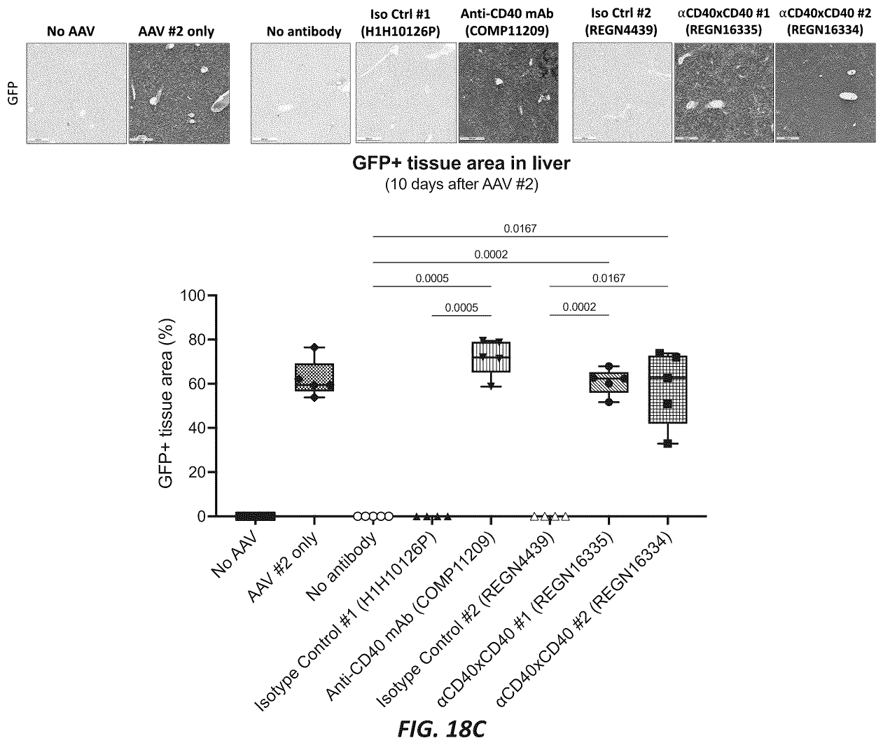

To determine whether the lower anti-AAV antibody titers observed in anti-CD40 antibody-treated mice were sufficient to enable AAV8 vector re-administration, mice were treated ˜3 months later with a second AAV8 vector (“AAV #2,” AAV8 CAG eGFP) via intravenous retroorbital injection at 6.56e12 vg/kg. A higher dose was used relative to the first dose in order to achieve more even liver transduction due to the relatively low levels of transduction for this vector in Example 11 at the lower 6.56e11 vg/kg dose (even in the positive control group). Ten days after treatment, mice were sacrificed, and eGFP transgene DNA and mRNA levels were evaluated in liver by quantitative real-time PCR (qPCR). As shown in FIGS. 18A-18B, mice treated with anti-CD40×CD40 bispecific Abs or anti-CD40 mAb showed detectable GFP transgene DNA and mRNA at levels comparable to transduction control mice receiving only AAV #2 (previously AAV-naive). To evaluate eGFP protein expression, eGFP immunohistochemical (IHC) analysis was performed on formalin-fixed, paraffin-embedded liver tissue, and eGFP+ area was quantified using HALO software (Indica Labs). IHC analysis showed that eGFP protein expression was significantly higher in anti-CD40×CD40 antibody and anti-CD40 mAb-treated mice versus mice that received the corresponding isotype controls, and comparable to levels in mice that received the eGFP vector only (FIG. 18C). These data show that antibody-mediated CD40 blockade can enable systemic AAV re-transduction at levels equivalent to previously AAV-naive animals.

### Example 14. Impact of Prophylactic CD40 Blockade on Formation of Germinal Center B Cell Responses

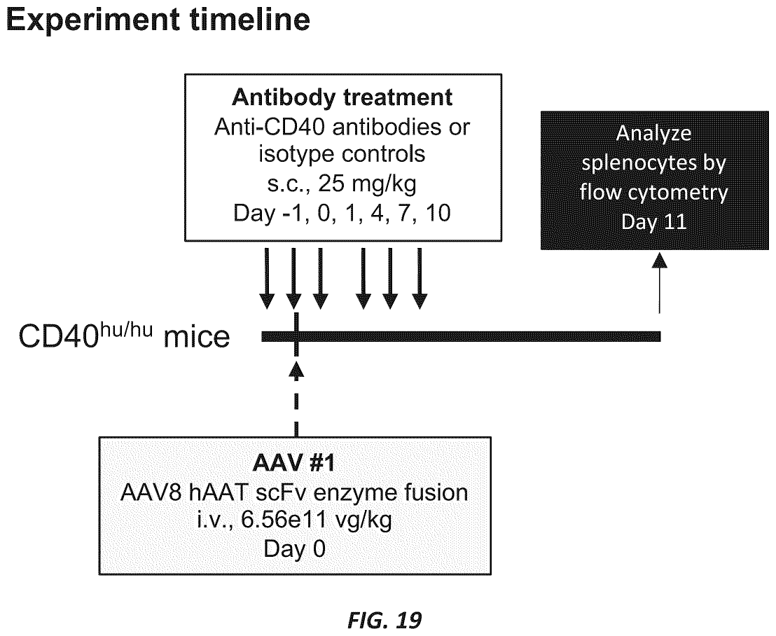

Productive antibody responses to T cell-dependent antigens, including formation of long-lived B cell immunological memory and plasma cells, depend on repeated CD40-CD40L interactions between a subset of activated B cells, known as germinal center (GC) B cells, and specialized CD4+ helper T cells known as follicular T helper cells (TFH). In this Example, whether antibody-mediated CD40 blockade can mechanistically ablate AAV-induced GC B cell responses was evaluated. Specifically, a study was conducted in CD40hu/hu mice in which mice were treated with AAV8 vector intravenously (6.56e11 vg/kg, i.v.) in the presence of anti-CD40×CD40 antibody, anti-CD40 mAb, or respective isotype control (outlined in FIG. 19; n=6 mice per group). Eleven days after treatment with AAV, mice were sacrificed for flow cytometry analysis of total and AAV-specific GC B cells in spleen. Specifically, single-cell splenocyte suspensions were prepared by mechanical disruption. Cells were transferred to a 96-well U-bottom plate, centrifuged at 400×g for 4 minutes and stained with a fixable viability dye for 15 minutes at room temperature. Cells were washed and incubated in Fc block for 15 minutes at 4° C. For detection of AAV-specific B cells, splenocytes were incubated with AAV8 for 1 hour on ice, (MOI 10,000) to facilitate interactions between AAV particle and antigen-specific B cell receptors (BCRs), followed by detection with anti-AAV8 biotinylated antibody (Progen) and Streptavidin-PE conjugate. Additional antibody staining was conducted with one of two antibody mixes as shown in Table 44 for 30 minutes at 4° C. After staining, the cells were washed twice with a BSA- and EDTA-containing wash buffer, fixed with BD Cytofix (cat #554655) diluted 1:4 in PBS for 15 minutes, then resuspended in wash buffer and stored at 4° C. The cells were then acquired on a FACSymphony A5 instrument and analyzed using OMIQ software. GC B cells were gated as follows: Live cells→Dump−(TCRb, CD200R3, Ly6G, CD49b and CD11b)→non-marginal zone B cells (CD1d−)→CD19+B220+→CD38-IgD−→GC positive (GL7+CD95+)→AAV+/−.

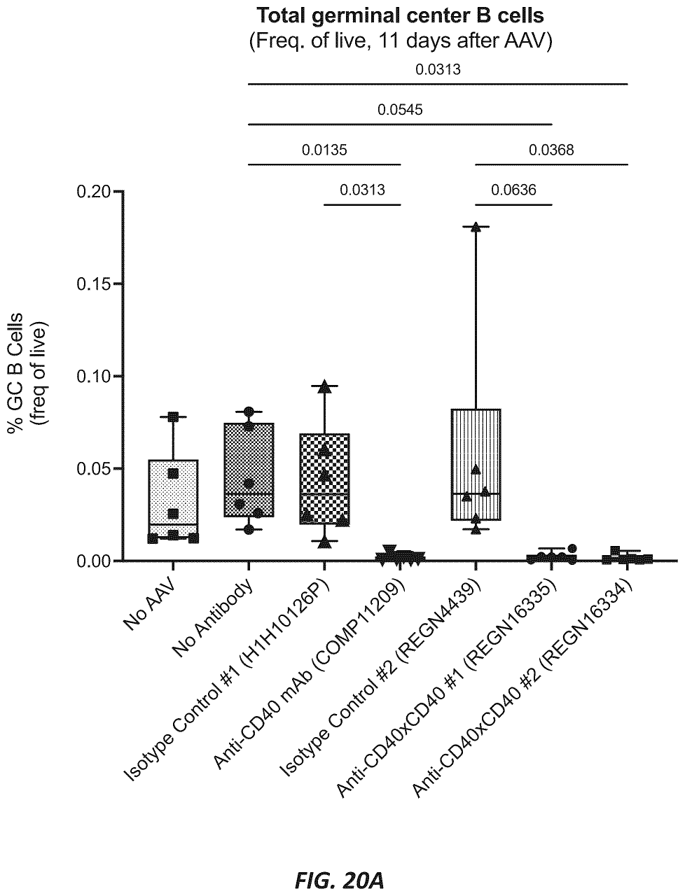

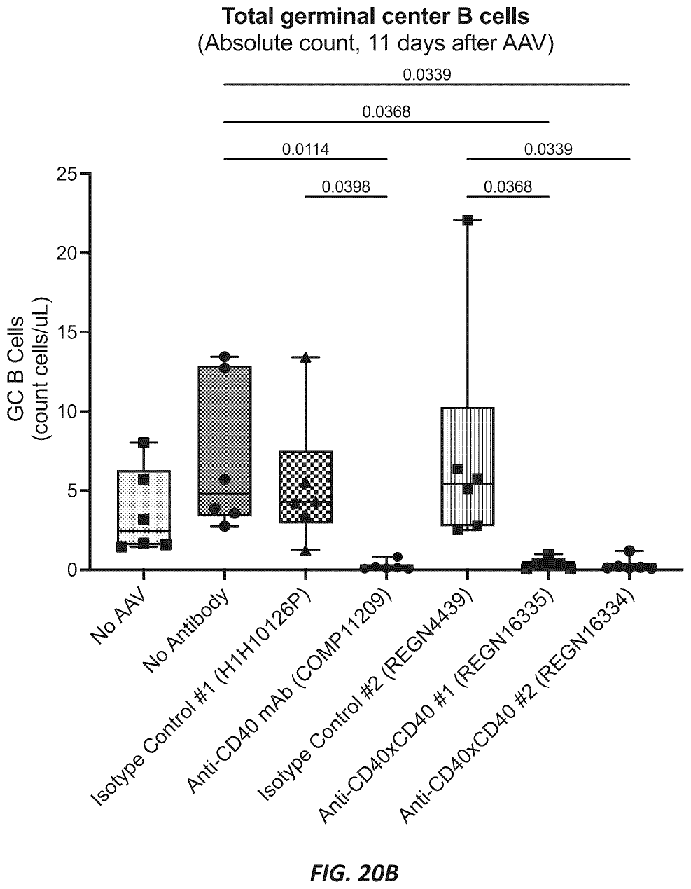

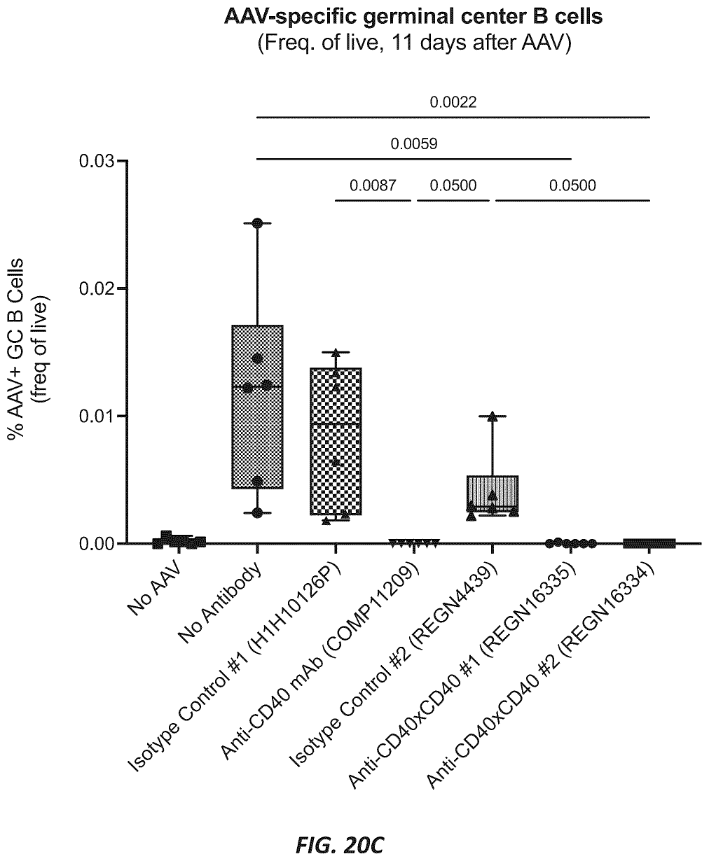

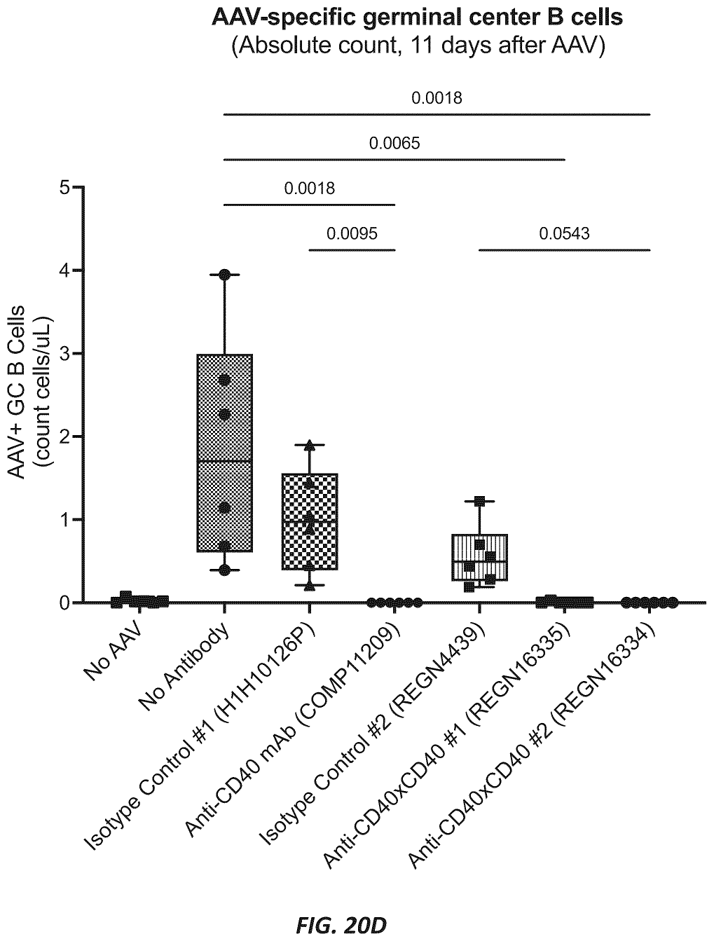

Analysis of total and AAV-specific GC B cells revealed that mice treated with anti-CD40×CD40 bispecific antibody or anti-CD40 mAb showed substantially reduced total GC and AAV-specific GC B cells relative to mice treated with relevant isotype controls or no antibody (FIGS. 20A-20D). Therefore, antibody-mediated CD40 blockade can prevent the formation of AAV-specific GCs, and suppress existing GC reactions.

TABLE 44
Description of Antibodies Used for Cell Staining.

| | | | | | | |
|---|---|---|---|---|---|---|
| Name/Antigen | Conjugate | Reactivity | Host | Clone | Isotype | Supplier |

Pre-Stain

| | | | | | | |
|---|---|---|---|---|---|---|
| Live/Dead | Blue | N/A | N/A | N/A | N/A | Invitrogen |
| Fc Block (CD16/32) | None | Human | Rat | 2.4G2 | N/A | TONGO biosciences |

Surface stain

| | | | | | | |
|---|---|---|---|---|---|---|
| B220 | BUV395 | Mouse | Rat | RA3-6B2 | IgG2a, κ | BD |
| CD3 | BUV805 | Mouse | Rat | 17A2 | IgG2b, κ | BD |
| CD40L | BUV737 | Mouse | Armenian Hamster | MR1 | IgG3, κ | BD |
| OX40 | BV786 | Mouse | Rat | OX-86 | AO IgG1, κ | BD |
| CD25 | BV510 | Mouse | Rat | PC61 | IgG1, λ | Biolegend |
| PD-1 | PE-Cy7 | Mouse | Rat | RPM1-30 | IgG2b, κ | Biolegend |
| CD44 | APC-Cy7 | Mouse | Rat | IM7 | IgG2b, κ | Biolegend |
| ICOS | APC | Mouse | Syrian Hamster | 15F9 | IgG | Biolegend |
| CD4 | BUV496 | Mouse | Rat | GK1.5 | IgG2b, κ | BD |
| CXCR5 | BV605 | Mouse | Rat | L138D7 | IgG2b, κ | Biolegend |
| CD8 | Alexa Fluor 488 | Mouse | Rat | 53-6.7 | IgG2a, κ | BD |
| CD19 | BV711 | Mouse | Rat anti-Mouse | 1D3 | IgG2a κ | BD |

Intracellular stain

| | | | | | | |
|---|---|---|---|---|---|---|
| FOXP3 | PerCP Cy 5.5 | Mouse | Rat | FJK-16s | IgG2a κ | eBioscience |
| Ki-67 | PE | Human/Mouse | Mouse | B56 | IgG1, κ | BD |

### Example 15. Impact of Prophylactic CD40 Blockade Using Anti-CD40 Antibodies on Generation of Anti-AAV T Cell Responses

In addition to its roles in regulating B cell immune responses, CD40-CD40L interactions are also known to participate in T cell immune responses via licensing of professional antigen presenting cells, which constitutively express CD40. In the context of AAV, mice deficient for CD40L, or mice administered CD40L blocking antibodies, show reduced T cell responses to AAV (Mays et al 2009, Shirley et al 2020). In this Example, the impact of antibody-mediated blockade of CD40 receptor on the T cell response immune response to AAV is evaluated. Specifically, CD40^{hu/hu} mice were treated prophylactically with anti-CD40×CD40-bispecific antibody, anti-CD40 mAb, or corresponding isotype control. Mice were subsequently treated with an AAV8 vector (as outlined in Example 14 and FIG. 19) and systemic T cell responses were evaluated in spleen by flow cytometry and IFNg ELISpot assay 11 days later. Flow cytometry analysis was conducted essentially as in Example 14, except that cells were stained with the antibodies indicated in Table 45. Additionally, for analysis of proliferating T cells, on the day of acquisition, the cells were washed with BD Perm/wash, incubated in BD Perm/wash buffer (cat #554723) for 20 minutes and stained with antibody against the proliferation marker Ki-67, for 30 minutes. Cells were subsequently washed twice and fixed with BD Cytofix. The cells were then acquired on a FACSymphony A5 instrument and analyzed using OMIQ software. TFH cells were gated as follows: Live→CD19−B220−→CD8−CD4+→CD44+→PD-1hi CXCR5hi. Proliferating CD4+ T cells were gated as follows: Live→CD19−B220−→CD8−CD4+→Ki-67+. IFNg ELISpot analysis was carried out essentially as described in the manufacturer's protocol (Mabtech), except that 5e5 splenocytes were plated per well in duplicate, and cells were stimulated with a peptide pool containing overlapping 15-mer peptides spanning the entire VP1 sequence of AAV8 (Pepmix AAV8 JPT Peptide Technologies GmbH, Berlin, Germany), 5 g/peptide.

TABLE 45
Description of Antibodies Used for Cell Staining.

| | | | | | | |
|---|---|---|---|---|---|---|
| Name/Antigen | Conjugate | Reactivity | Host | Clone | Isotype | Supplier |

Pre-Stain

| | | | | | | |
|---|---|---|---|---|---|---|
| Live/Dead | Blue | N/A | N/A | N/A | N/A | Invitrogen |
| Fc Block (CD16/32) | None | Human | Rat | 2.4G2 | N/A | TONGO biosciences |

Surface stain

| | | | | | | |
|---|---|---|---|---|---|---|
| B220 | BUV395 | Mouse | Rat | RA3-6B2 | IgG2a, κ | BD |
| CD3 | BUV805 | Mouse | Rat | 17A2 | IgG2b, κ | BD |
| CD40L | BUV737 | Mouse | Armenian Hamster | MR1 | IgG3, κ | BD |
| OX40 | BV786 | Mouse | Rat | OX-86 | AO IgG1, κ | BD |
| CD25 | BV510 | Mouse | Rat | PC61 | IgG1, λ | Biolegend |
| PD-1 | PE-Cy7 | Mouse | Rat | RPM1-30 | IgG2b, κ | Biolegend |
| CD44 | APC-Cy7 | Mouse | Rat | IM7 | IgG2b, κ | Biolegend |
| ICOS | APC | Mouse | Syrian Hamster | 15F9 | IgG | Biolegend |
| CD4 | BUV496 | Mouse | Rat | GK1.5 | IgG2b, κ | BD |
| CXCR5 | BV605 | Mouse | Rat | L138D7 | IgG2b, κ | Biolegend |
| CD8 | Alexa Fluor 488 | Mouse | Rat | 53-6.7 | IgG2a, κ | BD |
| CD19 | BV711 | Mouse | Rat anti-Mouse | 1D3 | IgG2a κ | BD |

Intracellular stain

| | | | | | | |
|---|---|---|---|---|---|---|
| FOXP3 | PerCP Cy 5.5 | Mouse | Rat | FJK-16s | IgG2a κ | eBioscience |
| Ki-67 | PE | Human/Mouse | Mouse | B56 | IgG1, κ | BD |

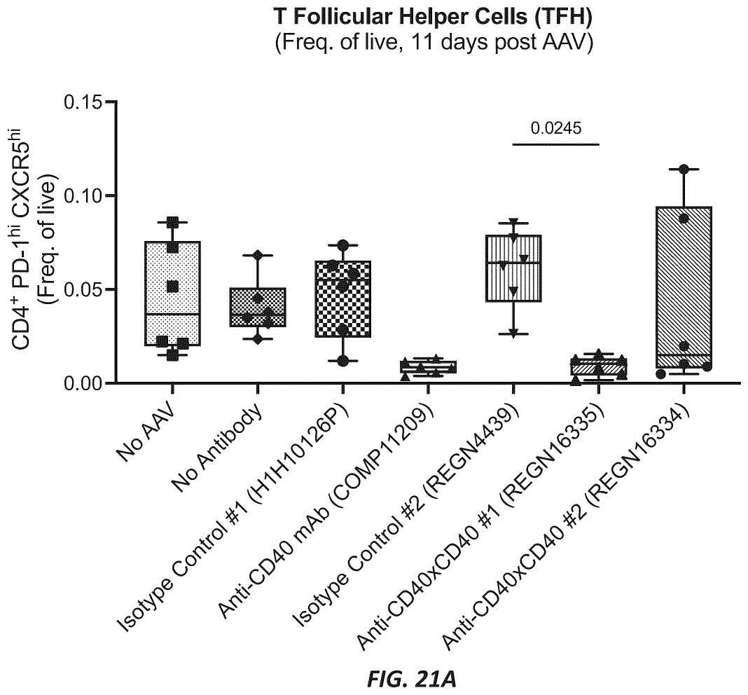

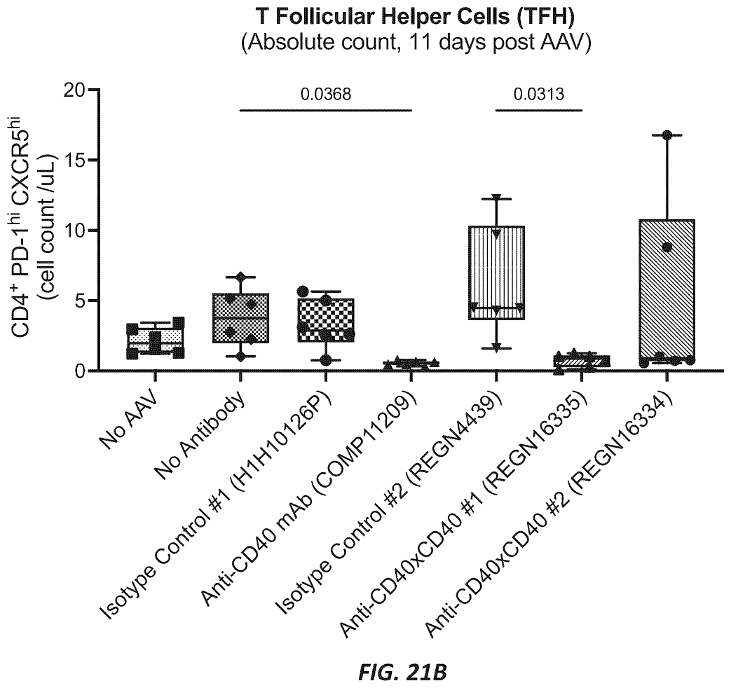

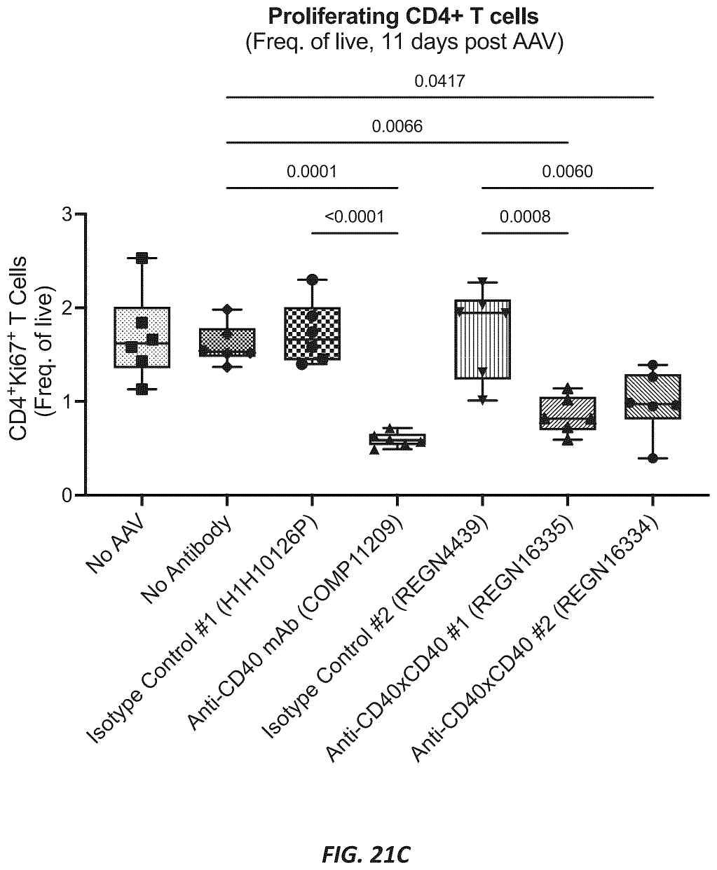

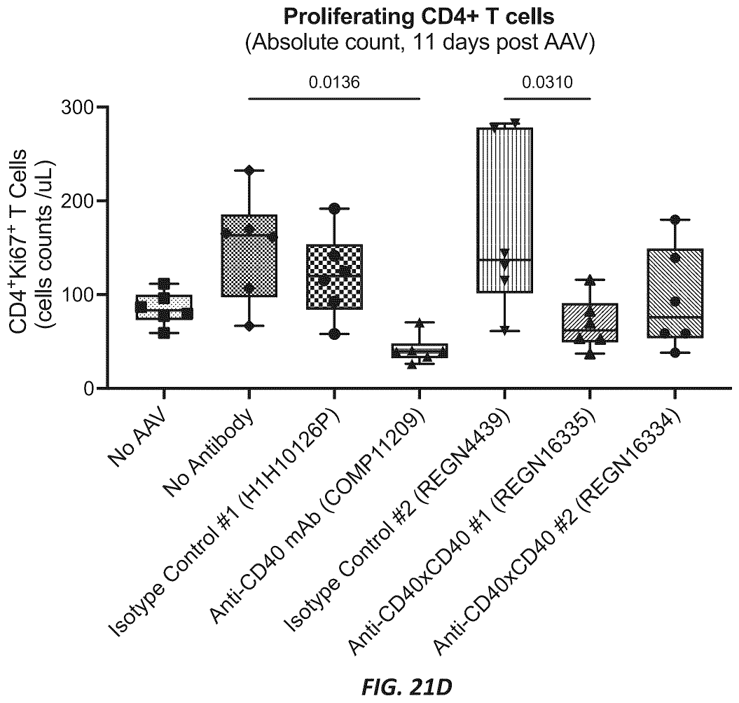

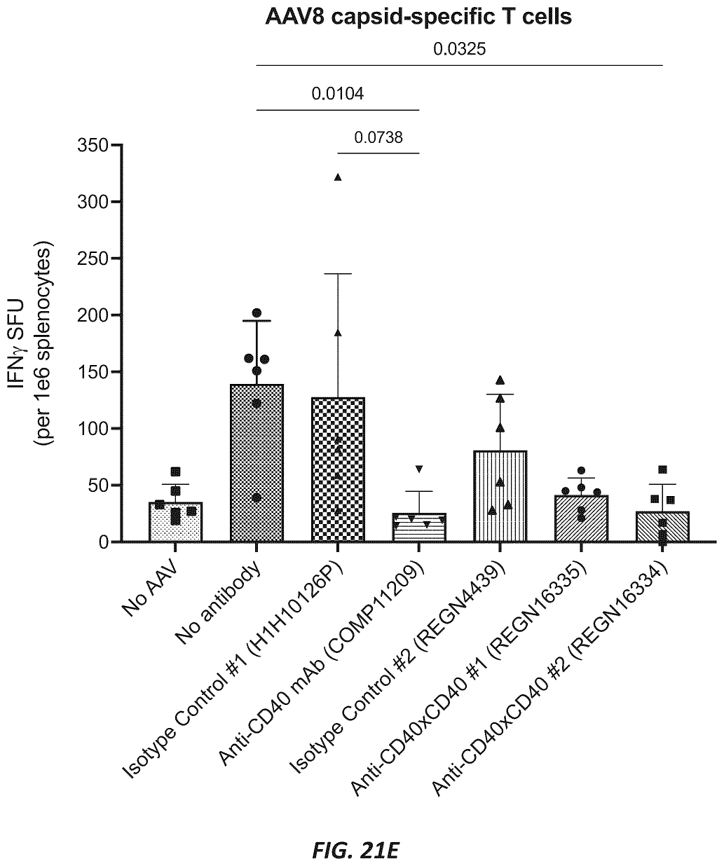

Flow cytometry analysis of splenic T cells from mice treated with anti-CD40×CD40 bispecific antibody or anti-CD40 mAb revealed strong suppression of total numbers and frequencies of follicular helper T cells (TFH), essential regulators of B cell germinal center responses (FIGS. 21A-21B), relative to mice treated with isotype controls or no antibody. Moreover, a more general reduction in number and frequency of proliferating CD4+ T cells (Ki-67+; FIGS. 21C-21D) was also observed. Analysis of antigen-specific T cell responses by IFNg ELISpot revealed suppression of anti-capsid T cell responses in anti-CD40×CD40 bispecific antibody and anti-CD40 mAb-treated animals versus isotype control mice or mice that were not treated with antibody (FIG. 21E). Thus, prophylactic antibody-mediated CD40 blockade can also be effectively utilized to suppress T cell responses to AAV, and may therefore represent an effective strategy to prevent elimination of transduced cells, or related pathology, resulting from T cell immune responses to transgene or capsid.

### Example 16. Impact of Prophylactic CD40 Blockade Using Anti-CD40 Antibodies on Development of Anti-Transgene Antibody Responses

Antibody responses to transgene products may develop following AAV gene therapy, and reduce therapeutic efficacy by impacting transgene product biodistribution or activity. To assess whether antibody-mediated CD40 blockade also prevents development of anti-transgene antibody responses, mice were treated with an AAV8 vector encoding a human lysosomal enzyme fused to an scFv antibody fragment (anti-CD63:GAA), in the presence or absence of anti-CD40×CD40 bispecific antibodies, anti-CD40 mAb, or corresponding isotype control antibodies, as described in Example 12 and FIG. 13. Anti-transgene antibody titers were then monitored in serum samples collected in the weeks following AAV treatment, essentially as described in Example 12, except that the ELISA plates were coated with the relevant recombinant human lysosomal enzyme scFv fusion protein (anti-CD63:GAA).

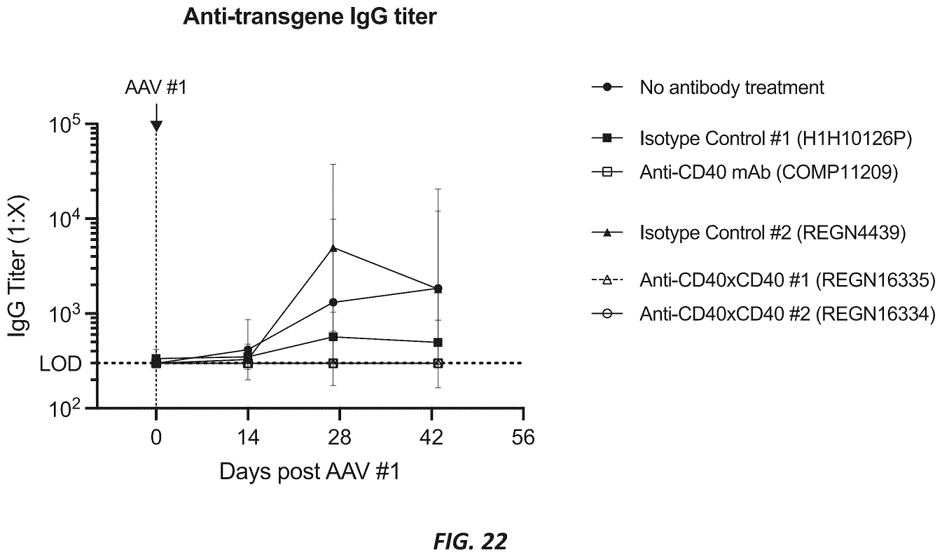

While mice treated with either no antibody or isotype control developed mild to moderate IgG responses against the human lysosomal enzyme scFv fusion transgene protein (anti-CD63:GAA), anti-GAA IgG responses were fully suppressed in mice that received anti-CD40×CD40 bispecific or anti-CD40 mAb blocking antibodies (FIG. 22). Thus, these data suggest that antibody-mediated CD40 blockade may also be utilized to prevent undesired antibody responses to transgene product.

Examples herein provide support for use of anti-CD40 receptor blocking antibodies to enable administration of a second AAV vector (“vector re-administration”). While there is some evidence to suggest that partial transduction with a second AAV vector may be achieved with antibody blockade of the ligand (CD40L), that the use of CD40L deficient mice may improve vector re-administration, and that anti-CD40L antibodies may suppress antibody responses to other vectors, in each instance, CD40L deficiency/blockade either failed to adequately suppress antibody responses to the vector, or failed to achieve transgene levels on second vector administration that were comparable to that of naive animals receiving AAV for the first time. By contrast, the data with anti-CD40×CD40 bispecific and anti-CD40 mAb blocking antibodies described herein shows that antibody responses to AAV capsid are strongly attenuated and that transgene expression is achieved at levels similar to that of naive animals on vector re-administration. Antibody responses to the transgene are also completely attenuated.

Examples herein further provide support for use of anti-CD40 receptor blocking antibodies to attenuate T cell immune responses to AAV.

Based on studies described herein, without wishing to be bound by theory, the mechanism of action of T cell inhibition can be through inhibition of antigen presenting cell (APC) licensing, including licensing of dendritic cells, macrophages, and B cells.

### Example 17. CryoEM Analysis of CD40×CD40 Bispecific Antibodies

To better understand the binding of REGN16335 and REGN16334 to CD40, structural analysis was performed via cryo-electron microscopy (cryo-EM). Fab fragments were prepared enzymatically. The Fab fragment 30027P2 corresponds to arm 1 of REGN16335, the Fab fragment 21519P2 corresponds to arm 1 of REGN16634, and the Fab fragment 21520P2 corresponds to arm 2 for both REGN16335 and REGN16334.

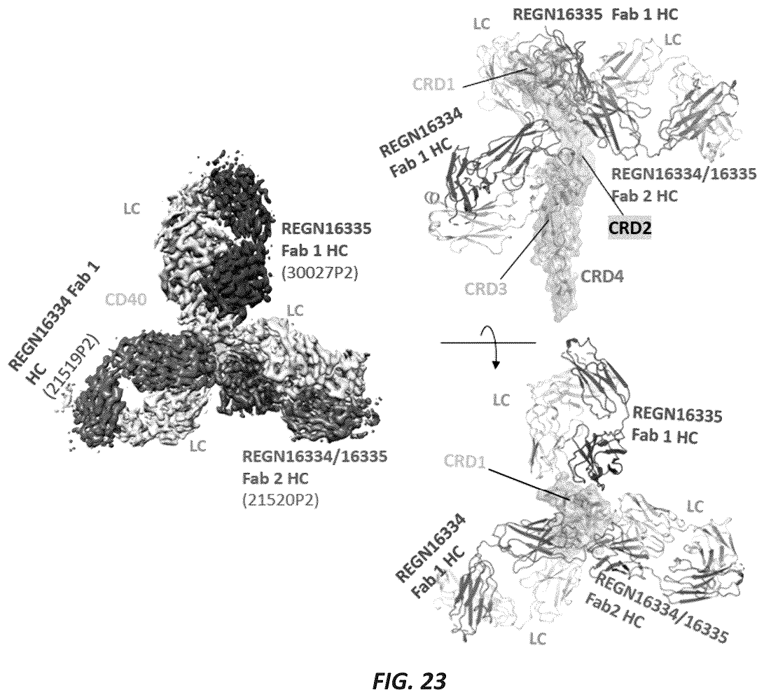

A 3D reconstructed map of the complex of CD40 with the three Fab arms (30027P2, 21519P2, and 21520P2) with resolution of 3 A shows that the three arms bind non-overlapping epitopes spanning the CRD1, CRD2, and CRD3 domains (FIG. 23). The 30027P2 Fab mostly binds the CRD1 domain and a small portion of CRD2, the 21520P2 Fab binds across CRD1 and CDR2 domains, and the 21519P2 Fab binds across CRD2 and CRD3 domains.

### Example 18. CD40 Antibody Sequences

TABLE 46
CD40 Antibody Sequences.

| SEQ ID NO | Description |
|---|---|
| 1 | 30027P2 HCVR nucleic acid sequence |
| 2 | 30027P2 HCVR amino acid sequence |
| 3 | 30027P2 HCDR1 nucleic acid sequence |
| 4 | 30027P2 HCDR1 amino acid sequence |
| 5 | 30027P2 HCDR2 nucleic acid sequence |
| 6 | 30027P2 HCDR2 amino acid sequence |
| 7 | 30027P2 HCDR3 nucleic acid sequence |
| 8 | 30027P2 HCDR3 amino acid sequence |
| 9 | Common LCVR nucleic acid sequence |
| 10 | Common LCVR amino acid sequence |
| 11 | Common LCDR1 nucleic acid sequence |
| 12 | Common LCDR1 amino acid sequence |
| 13 | Common LCDR2 nucleic acid sequence |
| | (GCTGCATCC) |
| 14 | Common LCDR2 amino acid sequence (AAS) |
| 15 | Common LCDR3 nucleic acid sequence |
| 16 | Common LCDR3 amino acid sequence |
| 17 | 30027P2 heavy chain nucleic acid sequence |
| 18 | 30027P2 heavy chain amino acid sequence |
| 19 | Common light chain nucleic acid sequence |
| 20 | Common light chain amino acid sequence |
| 21 | 21519P2 HCVR nucleic acid sequence |
| 22 | 21519P2 HCVR amino acid sequence |
| 23 | 21519P2 HCDR1 nucleic acid sequence |
| 24 | 21519P2 HCDR1 amino acid sequence |
| 25 | 21519P2 HCDR2 nucleic acid sequence |
| 26 | 21519P2 HCDR2 amino acid sequence |
| 27 | 21519P2 HCDR3 nucleic acid sequence |
| 28 | 21519P2 HCDR3 amino acid sequence |
| 29 | 21519P2 heavy chain nucleic acid sequence |
| 30 | 21519P2 heavy chain amino acid sequence |
| 31 | 21520P2 HCVR nucleic acid sequence |
| 32 | 21520P2 HCVR amino acid sequence |
| 33 | 21520P2 HCDR1 nucleic acid sequence |
| 34 | 21520P2 HCDR1 amino acid sequence |
| 35 | 21520P2 HCDR2 nucleic acid sequence |
| 36 | 21520P2 HCDR2 amino acid sequence |
| 37 | 21520P2 HCDR3 nucleic acid sequence |
| 38 | 21520P2 HCDR3 amino acid sequence |
| 39 | 21520P2 heavy chain nucleic acid sequence |
| 40 | 21520P2 heavy chain amino acid sequence |
| 41 | REGN16334 D1 heavy chain nucleic acid sequence |
| 42 | REGN16334 D1 heavy chain amino acid sequence |
| 43 | REGN16334 and REGN16335 D2 heavy |
| | chain nucleic acid sequence |
| 44 | REGN16334 and REGN16335 D2 heavy |
| | chain amino acid sequence |
| 45 | REGN16335 D1 heavy chain nucleic acid sequence |
| 46 | REGN16335 D1 heavy chain amino acid sequence |
| 47 | REGN16431 D1 heavy chain nucleic acid sequence |
| 48 | REGN16431 D1 heavy chain amino acid sequence |
| 49 | REGN16431 and REGN16432 D2 heavy |
| | chain nucleic acid sequence |
| 50 | REGN16431 and REGN16432 D2 heavy |
| | chain amino acid sequence |
| 51 | REGN16432 D1 heavy chain nucleic acid sequence |
| 52 | REGN16432 D1 heavy chain amino acid sequence |
| 53 | REGN3094 (human CD40 extracellular domain |
| | with C-terminal MMH tag; “hCD40-MMH”) |
| 54 | REGN3097 (monkey CD40 extracellular domain |
| | with C-terminal MMH tag; “mfCD40-MMH”) |
| 55 | REGN3098 (mouse CD40 extracellular domain |
| | with C-terminal MMH tag; “mCD40-MMH”) |
| 56 | REGN3095 (hCD40-mFc) |
| 282 | REGN20484 D1 heavy chain nucleic acid sequence |
| 283 | REGN20484 D1 heavy chain amino acid sequence |
| 284 | REGN20484 D2 heavy chain nucleic acid sequence |
| 285 | REGN20484 D2 heavy chain amino acid sequence |

### Example 19. Step-Wise Increase in FIX Blood Levels by Repeated Gene Insertion of a FIX Transgene Template into a Single Locus in the Presence of Anti-CD40-Based Immunomodulation of Anti-AAV Immune Response

Using anti-CD40 antibodies or other CD40 inhibitors to mitigate an anti-AAV antibody response can allow for repeated dosing of an identical gene insertion therapeutic cargo. In this example, an AAV8 encoding a promoterless, bidirectional DNA template of hFIX and an LNP containing mRNA encoding SpCas9 and a G666 gRNA specific for creating a specific double stranded break in mouse albumin intron 1 are dosed multiple times while mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep), allowing for titrated, increasing insertion of a hFIX transgene template into the albumin locus of hepatocytes in mice. See, e.g., WO 2023/077012 and US 2023-0149563, each of which is herein incorporated by reference in its entirety for all purposes. This gene insertion allows hepatocytes to use the albumin locus to produce secreted hFIX in a step-wise, increasing fashion. Briefly, mice with native Alb^{+/+} are dosed with anti-CD40 antibody or other CD40 inhibitor before (e.g., 1 week before) and after (e.g., up to 6 months after) the 1^{st} AAV+LNP dosing (e.g., 3.33E11 vg/kg to 5E13 vg/kg AAV). This results in production of FIX from hepatocytes, and a stable level of FIX in blood. The anti-drug antibody titers are measured 4 weeks after dosing for AAV, and the titers are not significantly different from pre-treatment titers. The anti-CD40 antibody or other CD40 inhibitor prevents the production of neutralizing antibody titers against the AAV, and the AAV+LNP therapeutic is dosed again 4-12 weeks after the 1^{st} AAV+LNP dose. This results in a step-wise increase in blood FIX levels due to increased gene insertion in additional hepatocytes. Additional dosings of AAV+LNP for increased gene insertion are possible as long as the anti-CD40 antibody or other CD40 inhibitor is suppressing the host anti-AAV antibody immune response and there are hepatocytes containing a cut site for the gRNA for the hFIX transgene to be inserted.

### Example 20. Step-Wise Increase in FIX Blood Levels by Gene Insertion of FIX Transgene Template into Distinct Locations in Albumin 11t Intron Using Anti-CD40 Antibody Immunomodulation

Using anti-CD40 antibodies or other CD40 inhibitors to mitigate an anti-AAV antibody response can allow for gene insertion of an AAV template into two separate genomic locations from two discrete dosings of AAV and LNP. In this example, an AAV8 encoding a promoterless, bidirectional DNA template of hFIX and two distinct LNPs are dosed sequentially times while mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep), allowing for step-wise increasing hFIX levels in blood. This illustrates using non-overlapping, different sites to insert a FIX transgene to increase blood levels of a protein FIX.

The 1^{st} LNP (LNP G666) contains mRNA encoding SpCas9 and G666 gRNA for creating a double-stranded break in a specific location in mouse albumin intron 1. The 2^{nd} LNP contains mRNA encoding SpCas9 and a second gRNA for creating a double-stranded break in a specific location (non-overlapping with G666) in mouse albumin intron 1. Briefly, mice with native Alb^{+/+} are treated with anti-CD40 antibody or other CD40 inhibitor before and after the AAV+LNP G666 dosing. This results in production of FIX from hepatocytes, and a stable, detectable level of FIX in blood. The anti-drug antibody titers are measured 4 weeks after dosing for AAV after dosing, and they are not significantly different from pre-treatment titers. As the anti-CD40 antibody or other CD40 inhibitor prevents the production of neutralizing antibody titers against the AAV, AAV+LNP G555 therapeutic is dosed 4-12 weeks after. This results in a step-wise increase in blood FIX levels due to gene insertion of the hFIX template into the 1^{st} albumin intron in hepatocytes. This method ensures that if the CRISPR recognition sequence for the G666 insertion site for has been destroyed by an indel and thus prevents insertion, the 2^{nd} LNP can be used to insert in a different CRISPR recognition sequence to allow for additional FIX transgene insertion.

### Example 21. Sequential Gene Insertion of Two Different Gene Templates for Increasing FIX Levels Over Time Using Anti-CD40 Antibody Immunomodulation

Using anti-CD40 antibodies or other CD40 inhibitors to mitigate an anti-AAV antibody response can allow for gene insertion of two different AAV templates from two discrete dosings of AAV and LNP. In this example, an AAV (AAV #1) encoding a promoterless, bidirectional DNA template of hFIX and an LNP (LNP G666) that contains mRNA encoding SpCas9 and G666 gRNA for creating a double-stranded break in a specific location in mouse albumin intron 1 are used for the first dose in mice. Mice with native Alb^{+/+} are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep) before and after the AAV+LNP G666 dosing. FIX levels are detected in blood after dosing with the 1^{st} AAV+LNP. The anti-drug antibody titers are measured 4 weeks after dosing for AAV after dosing, and they are not significantly different from pre-treatment titers. 4-12 weeks later, a 2^{nd} AAV (AAV #2) encoding a differently codon-modified promoterless, bidirectional DNA template of hFIX and LNP G666 are dosed into mice. FIX levels are increased after dosing, indicating a different FIX template is inserted into mice hepatocytes but still yields increased FIX levels. PCR based quantitation methods are used to differentiate hepatocytes that contain the FIX template from AAV #1 and AAV #2, demonstrating that an additional AAV can be dosed for increasing gene insertion when the anti-AAV antibody response is suppressed.

### Example 22. Sequential Gene Insertion for Expression of Two Different Protein Products Using Anti-CD40 Antibody Immunomodulation

In this example, an AAV (AAV #1) encoding a promoterless, bidirectional DNA template of hFIX and an LNP (LNP G666) that contains mRNA encoding SpCas9 and G666 gRNA for creating a double-stranded break in a specific location in mouse albumin intron 1 are used for the first dose in mice. Mice with native Alb^{+/+} are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep) before and after the AAV+LNP G666 dosing. FIX levels are detected in blood after dosing with the 1^{st} AAV+LNP. 4-12 weeks later, a 2^{nd} AAV (AAV #2) encoding a promoterless DNA template for a secreted GAA fusion protein is dosed with LNP G666. FIX levels remain constant, but a new GAA fusion protein is detected in blood. PCR based quantitation methods are used to differentiate hepatocytes that contain the templates from AAV #1 and AAV #2, demonstrating that an additional AAV can be dosed for inserting different transgenes at different times into cells.

Alternatively, after the 1^{st} AAV+LNP insertion event, the 2^{nd} insertion event used AAV #2 with an LNP #2. The LNP #2 contains mRNA encoding SpCas9 and a gRNA specific for creating a double stranded break in intron 1 of a second target gene that is not Alb. This allows two insertions of two different transgenes controlled by different promoters.

### Example 23. Sequential Gene Insertion of a Gene Template for Increasing Therapeutic Antibody Transgene Levels Over Time Using Anti-CD40 Antibody Immunomodulation in a Target Tissue

In this example, an AAV encoding a promoterless, bidirectional DNA template of a therapeutic antibody and an LNP that contains mRNA encoding SpCas9 and a gRNA is used. The LNP creates a double stranded break in a specific location in an intron of a transgene downstream of a promoter with activity in the target tissue. The AAV and LNP combination is co-administered to the target tissue, and mice are concurrently dosed subcutaneously with anti-CD40 antibody (e.g., REGN16335, iscalimab, ravagalimab, BI-655064, or abiprubart) or other CD40 inhibitor (e.g., CD40L binding protein such as tegoprubart of dazodalibep). Therapeutic antibody is detected in blood within 1 week of AAV+LNP dosing. Anti-AAV capsid and anti-antibody titers are measured, and they are not significantly different than pre-treatment titers. An additional AAV+LNP dose is administered 4-12 weeks after the 1^{st} AAV+LNP dose in another target tissue location. The therapeutic antibody levels are measured in blood, and they are greater than before the 2^{nd} AAV+LNP administration. This demonstrates that the therapeutic antibody is able to be increased in a step-wise fashion due to repeatable gene insertion administration.

### Example 24. Anti-Transgene Antibody and Cytotoxic T Cell Responses are Reduced Following a Gene Insertion Using Anti-CD40 Antibody Immunomodulation

In this example, an AAV8 encoding a promoterless, bidirectional DNA template of a secreted GAA fusion protein and an LNP containing mRNA encoding SpCas9 and a G666 gRNA specific for creating a specific double stranded break in mouse albumin intron 1 are dosed into mice. Prior to and during the time after AAV+LNP dosing, mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep). GAA fusion protein is detected in serum after 1-2 weeks of dosing and remains stable throughout the 12 week study. Anti-GAA antibodies are measured 4 weeks and 12 weeks after the gene insertion dosing, and they are not significantly different from negative control mice that do not receive AAV, showing a lack of anti-transgene antibody development. Additionally, at necropsy, IFNg ELISpots conducted on splenocytes show significantly reduced T cell immune responses against both AAV capsid and GAA fusion protein. Thus, CD40 blockade can mitigate anti-transgene antibody responses, as well as T cell immune responses against AAV capsid and transgene, following AAV gene insertion.

### Example 25. Combination of Anti-BCMA×CD3 to Eliminate Pre-Existing Immunity to AAV and CD40 Blockade to Prevent New Antibody Response to AAV on Subsequent AAV Exposure

In this example, anti-BCMA×CD3 is used to eliminate pre-existing immunity to AAV, while anti-CD40 antibody (e.g., REGN16335, iscalimab, ravagalimab, BI-655064, or abiprubart) or other CD40 inhibitor (e.g., CD40L binding protein such as tegoprubart of dazodalibep) is utilized to prevent any new antibody response to AAV on subsequent AAV exposure. Specifically, in individuals with pre-existing antibody responses to AAV, anti-BCMA×CD3, given either alone or in combination with FcRn blockade (or similar IgG depletion strategy) and/or B cell depletion, is initiated to reduce an anti-AAV antibody response. Once anti-AAV antibodies reach sub-neutralizing levels, anti-CD40 antibody (e.g., REGN16335, iscalimab, ravagalimab, BI-655064, or abiprubart) or other CD40 inhibitor (e.g., CD40L binding protein such as tegoprubart of dazodalibep) treatment is started, and a therapeutic AAV (such as an AAV8 hFIX gene insertion therapy) is dosed in the days or weeks thereafter. A new antibody response against AAV is thereby prevented. Thus, anti-BCMA×CD3 returns an individual with pre-existing antibody immunity to an effectively immunologically naive status, while CD40 blockade preserves naive status upon therapeutic AAV exposure, therefore allowing for AAV re-dosing in the future.

### Example 26. Combination of Anti-BCMA×CD3 to Eliminate Potential Residual Antibody Responses Following AAV Exposure in Presence of CD40 Blockade

In this example, anti-BCMA×CD3 is used to eliminate potential residual antibody responses generated following AAV exposure in the presence of CD40 blockade, thereby rescuing the ability to re-dose AAV. Specifically, CD40 blockade (e.g., with anti-CD40 antibody (e.g., REGN16335, iscalimab, ravagalimab, BI-655064, or abiprubart) or other CD40 inhibitor (e.g., CD40L binding protein such as tegoprubart of dazodalibep)) is initiated prior to AAV treatment (such as an AAV8 hFIX gene insertion therapy). After AAV exposure, reduced but measurable antibody titers are generated to the AAV capsid. Treatment with anti-BCMA×CD3, either alone or in combination with FcRn blockade (or similar IgG depletion strategy) and/or B cell depletion, is then initiated to bring residual antibody titers to sub-neutralizing levels. A second AAV therapeutic (either the same or different) is then administered, and transduction is successful. Thus, in scenarios where CD40 blockade attenuates but does not fully eliminate antibody titers to AAV, anti-BCMA×CD3 can be used to reduce titers to levels that enable AAV re-dosing.

### Example 27. Combination of CD40 Blockade Administered Concomitantly with Anti-BCMA×CD3 to Block Ongoing Antibody Responses to AAV from Recent Exposure

In this example, CD40 blockade (e.g., with anti-CD40 antibody (e.g., REGN16335, iscalimab, ravagalimab, BI-655064, or abiprubart) or other CD40 inhibitor (e.g., CD40L binding protein such as tegoprubart of dazodalibep)) is administered concomitantly with anti-BCMA×CD3 to block ongoing antibody responses to AAV from recent exposure. Specifically, in individuals with pre-existing antibody responses to AAV, anti-BCMA×CD3 is administered with anti-CD40 antibody in a single or repeated dose regimen. FcRn blockers (or similar IgG depletion strategy) may also be used. Whereas anti-BCMA×CD3 depletes antibody-secreting cell populations, CD40 blockade prevents new B cell activation and terminates ongoing B cell responses that would otherwise not be impacted by anti-BCMA×CD3 treatment alone. Thus, CD40 blockade enhances the therapeutic effect of anti-BCMA×CD3.

### Example 28. Plasma Cell Depletion in Combination with Neonatal Fe Receptor (FcRn) Blockade Elicits Rapid and Deep Suppression of Pre-Existing Anti-AAV Antibody Titers

For adeno-associated virus gene therapy, the generation of neutralizing antibodies after exposure precludes the ability to re-administer AAV vectors of the same or related serotypes, despite therapeutic need. Furthermore, due to natural exposure to wild-type AAVs, roughly 30-60% of individuals harbor pre-existing antibodies to AAV that prevent administration of even a single AAV vector. Therefore, strategies that can attenuate pre-existing anti-AAV antibody responses induced by either recombinant or wild type AAVs have the potential to greatly expand the versatility and accessibility of AAV gene therapies to a broader patient population. In some embodiments, the subsequently administered AAV vector has a capsid derived from the same AAV serotype as the originally administered AAV vector.

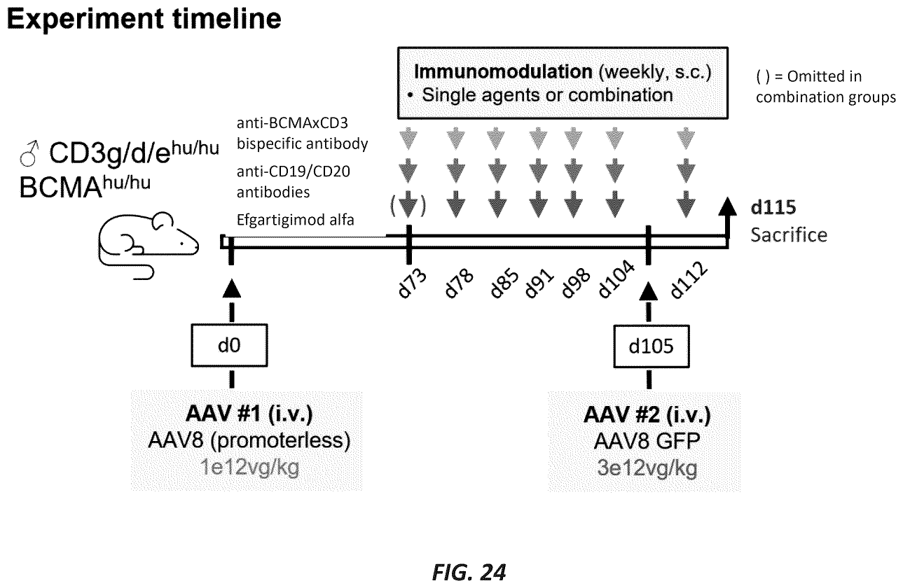

Because plasma cells are the source of long-lived antibody responses, it was reasoned that antibody-mediated plasma cell depletion may suppress pre-existing antibody responses to AAV sufficiently to enable AAV vector transduction or re-transduction in seropositive animals. To test this, B cell maturation antigen (BCMA)− and CD3 gamma−, CD3 delta−, and CD3 epsilon-humanized mice (n=6 per group) were treated with 1e12 vector genomes (vg) per kilogram (kg) recombinant AAV8 (encoding a promoterless transgene) to induce a strong anti-capsid antibody response (e.g., high-titer nAbs). 73 days later, a timepoint deemed sufficient to account for long-lived plasma cell differentiation, mice were injected subcutaneously weekly for five weeks with 25 milligrams (mg) per kg linvoseltamab, also known as REGN5458, a fully-human T cell-bridging bispecific antibody targeting B cell maturation antigen and CD3 (referred to herein as “anti-BCMA×CD3 bispecific antibody”) to induce plasma cell depletion. Additionally, because the half-life of immunoglobulin G is relatively long (˜6-8 days in mice and ˜21 days in humans) due to the action of neonatal Fc receptor (“FcRn”), it was also evaluated whether additional blockade of FcRn with efgartigimod alfa, administered subcutaneously weekly at 20 mg/kg, could further accelerate and improve titer reductions elicited by plasma cell depletion. Finally, to capture a wider range of AAV-specific B cells that may not express high levels of BCMA, such as committed memory B cells and early plasmablasts, it was also tested whether additional B cell depletion with a cocktail of anti-CD19/CD20 antibodies, administered subcutaneously weekly at 25 mg/kg each, may further improve the therapeutic effect of plasma cell depletion with anti-BCMA×CD3 bispecific antibody. Mice were bled at defined intervals for serum anti-AAV antibody analysis. A schematic of the full experimental design is presented in FIG. 24. To prevent any initial impact of efgartigimod alfa on the therapeutic effect of anti-BCMA×CD3 bispecific antibody or anti-CD19/CD20 antibodies, which are themselves immunoglobulins, efgartigimod alfa was omitted from the first week's treatment cocktails.

To evaluate the impact of plasma cell depletion, FcRn blockade, B cell depletion, or combinations thereof on anti-AAV8 IgG titers, anti-capsid IgG levels were measured in serum of mice over time. Specifically, 96 well flat-bottom plates were coated with 1e9 vg/well recombinant AAV8 vector in DPBS overnight. The next day, plates were washed and blocked with 0.5% bovine serum albumin in DPBS for 1 hr. Serum samples were then diluted 3×, beginning at an initial dilution of 1:300 and ending at a dilution of 53,144,100. Diluted serum was then transferred to the assay plate and incubated overnight at 4° C. The next day, the assay plates were repeatedly washed prior to incubation with an anti-mouse-IgG Fc-gamma Fragment-HRP-conjugated polyclonal secondary antibody (Jackson Immunoresearch, West Grove, PA). Plates were again repeatedly washed prior to development with TMB substrate solution. After 20 minutes, the reaction was stopped by addition of 2N phosphoric acid. Absorbance at 450 nm (OD450) was measured on a SpectraMax i3 plate reader (Molecular Devices, San Jose, CA). Relative levels of serum anti-AAV8 IgG were determined and plotted as titer values using Prism v.9 software (GraphPad, Boston, MA). Titer was defined as the dilution factor required to achieve an OD450 reading equal to 2-fold higher than background values.

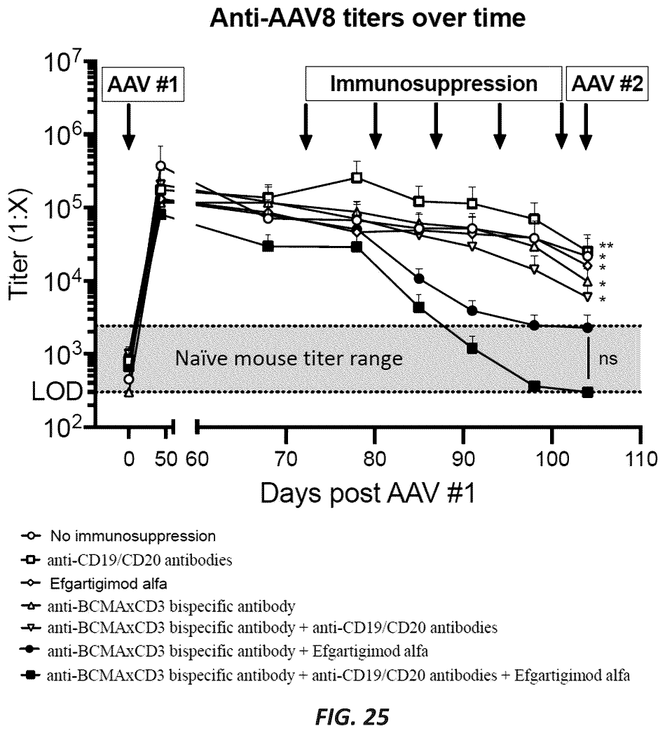

It was found that while anti-AAV8 antibody titers showed minor declines over time in mice administered either anti-BCMA×CD3 bispecific antibody, efgartigimod alfa, or anti-CD19/CD20 antibodies individually, the titer reductions were minor and not statistically different from AAV-treated mice that received no immunomodulation. By contrast, mice receiving a cocktail of anti-BCMA×CD3 bispecific antibody and efgartigimod alfa showed rapid titer declines to naive or near-naive levels, and mice additionally treated with anti-CD19/CD20 antibodies showed even more rapid and complete titer declines, with all 6/6 mice exhibiting titers below the limit of detection by the cessation of the five-week treatment period (FIG. 25). Thus, these data demonstrate that anti-AAV8 titers can be suppressed by therapeutic plasma cell depletion, with the timeframe required for anti-AAV8 titer depletion reduced by FcRn blockade. Additionally, the depth of titer reduction can be further enhanced with B cell depletion in mice.

### Example 29. Plasma Cell Depletion in Combination with FcRn Blockade Enables AAV Vector Re-Administration

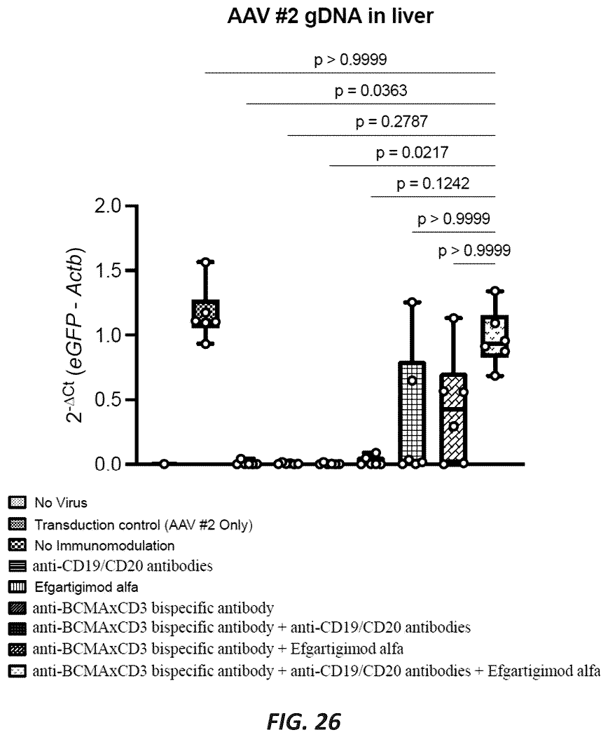

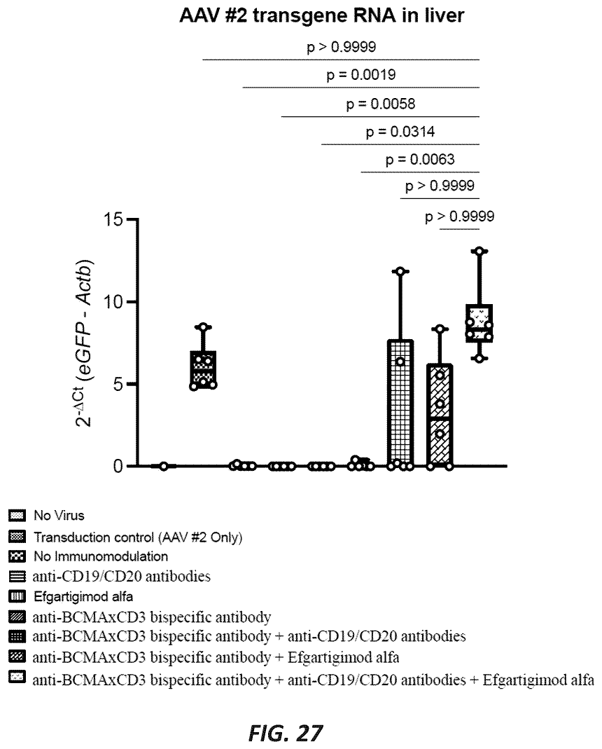

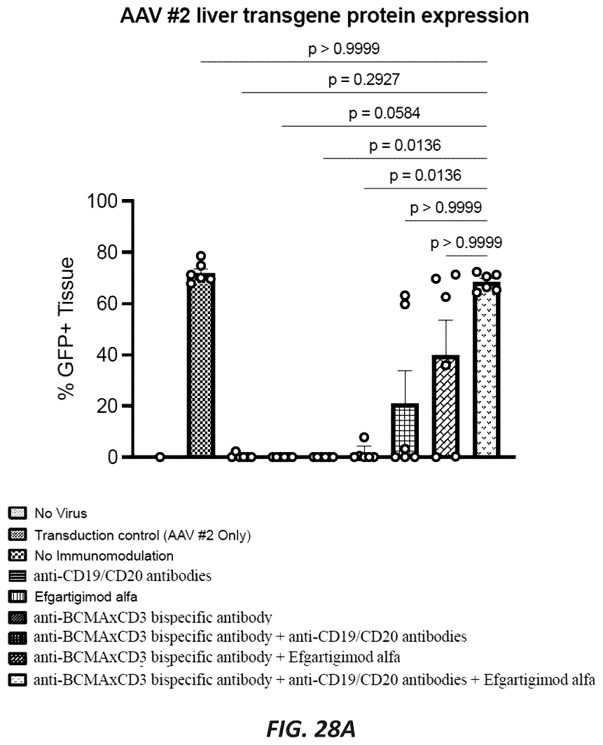

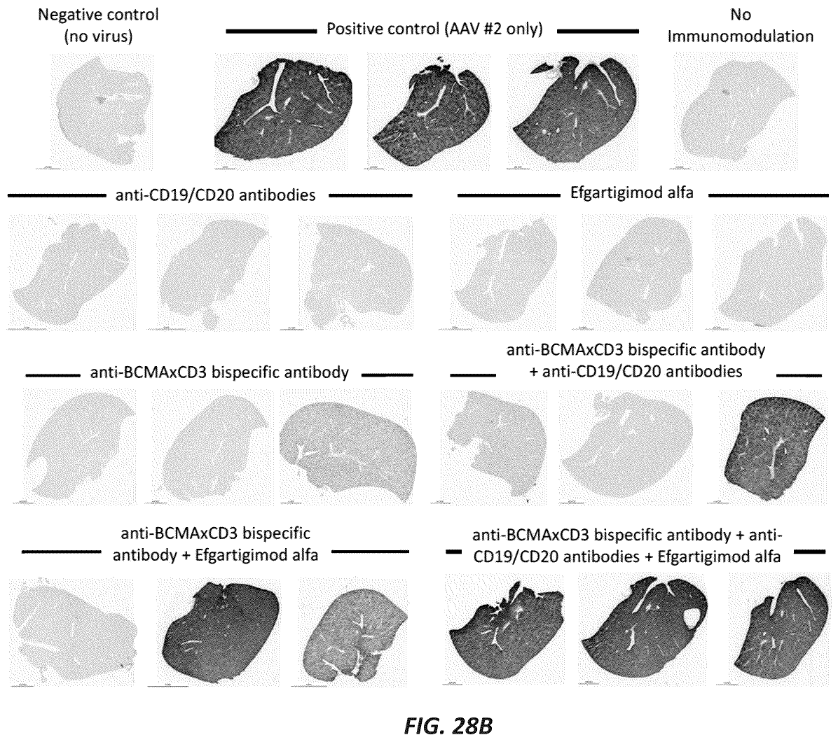

Next was evaluated whether the deep titer reductions observed in mice following combination treatment of anti-BCMA×CD3 bispecific antibody and efgartigimod alfa, and following combination treatment of anti-BCMA×CD3 bispecific antibody, efgartigimod alfa, and anti-CD19/CD20 antibodies, could enable re-transduction with a second AAV vector. To this end, the mice from Example 28 were treated intravenously with 3e12 vg/kg AAV8 GFP, then sacrificed 10 days later to evaluate transgene expression in liver (FIG. 24). While nearly all mice receiving no immunomodulation or single-agent immunomodulation failed to achieve any re-transduction in liver, due to presence of anti-AAV8 antibodies from previous AAV8 exposure, significant levels of GFP transgene DNA (FIG. 26) and RNA (FIG. 27) were observed in mice receiving anti-BCMA×CD3 bispecific antibody+FcRn blocker, and less frequently in mice receiving anti-BCMA×CD3 bispecific antibody+anti-CD19/CD20 antibodies, as measured by quantitative real-time PCR and quantitative real-time reverse transcription PCR, respectively. 3/6 mice receiving anti-BCMA×CD3 bispecific antibody+FcRn blocker achieved re-transduction equivalent to seronegative control mice. The greatest level of re-transduction was observed in the triple combination group, with 6/6 mice achieving transgene levels equivalent to that of previously naive mice. Immunohistochemical staining of formalin-fixed, paraffin-embedded liver sections for GFP transgene protein corroborated these findings (FIGS. 28A-28B). Together, these data indicate that anti-BCMA×CD3 bispecific antibody-mediated plasma cell depletion, particularly in combination with FcRn blockade, can enable AAV re-dosing, or other immunogenic gene therapy vectors, regardless of serostatus, and that the success rate for re-dosing may be further enhanced by B cell depletion in mice.

### Example 30. Analysis of Plasma Cell Frequencies and Counts in Spleen and Bone Marrow Following Anti-BCMA×CD3 Bispecific Antibody Treatment

To confirm on-target activity of anti-BCMA×CD3 bispecific antibody, plasma cell numbers in bone marrow and spleen were evaluated by flow cytometry at the time of sacrifice (FIG. 24). Specifically, single-cell splenocyte suspensions were prepared by mechanical disruption of spleen. For bone marrow extraction, femurs were cut at both ends, placed in a PCR plate with holes punched at the bottom, and spun down for 3 minutes at 500 g. Red blood cells were lysed using ACK lysis buffer. Cells were transferred to a 96 well U-bottom plate, centrifuged at 400 g for 4 minutes and stained with LIVE/DEAD Fixable Blue Dead Cell dye (ThermoFisher) for 15 minutes at room temperature. Cells were washed and incubated in Fc block (Tonbo Biosciences) for 15 to 30 minutes at 4° C. For detection of AAV-specific B cells, splenocytes were incubated with recombinant AAV8 for 1 hr on ice, (multiplicity of infection [MOI] of 10,000) to facilitate interactions between AAV particles and antigen-specific BCRs, followed by washing and labeling with anti-AAV8 biotinylated antibody (clone ADK8, Progen) and surface stain antibody cocktail (Table 47) for 30 minutes at 4° C. in Brilliant Stain buffer (BD Biosciences). Cells were again washed and then stained with Streptavidin-PE conjugate (Biolegend) for an additional 20 minutes at 4° C., followed by washing and fixation with BD Cytofix (BD Biosciences). For intracellular staining, samples were washed and incubated in 1× Perm/Wash buffer (BD Biosciences) for 20 minutes and resuspended in intracellular stain (Table 47) for 30 minutes at 4° C. followed by washing and fixation with BD Cytofix (BD Biosciences). CountBright Absolute counting Beads (ThermoFisher) were used according to the manufacturer's protocol to enumerate absolute cell counts. Acquisition was performed on a BD FACSymphony A5 using FACSDiva software. Analysis was performed using FlowJo or OMIQ software. All B cells were first gated according to light scatter properties, then negatively gated to exclude viability dye positive and non-B cell lineage marker positive cells. Specific B cell populations were then gated as follows: Naive B cells, CD19+B220+CD1d−IgD+CD38+; Memory B cells, CD19+B220+CD1d−IgD−CD38+AAV+/−; Plasma cells: B220−IgD−CD138+ Light Chain+.

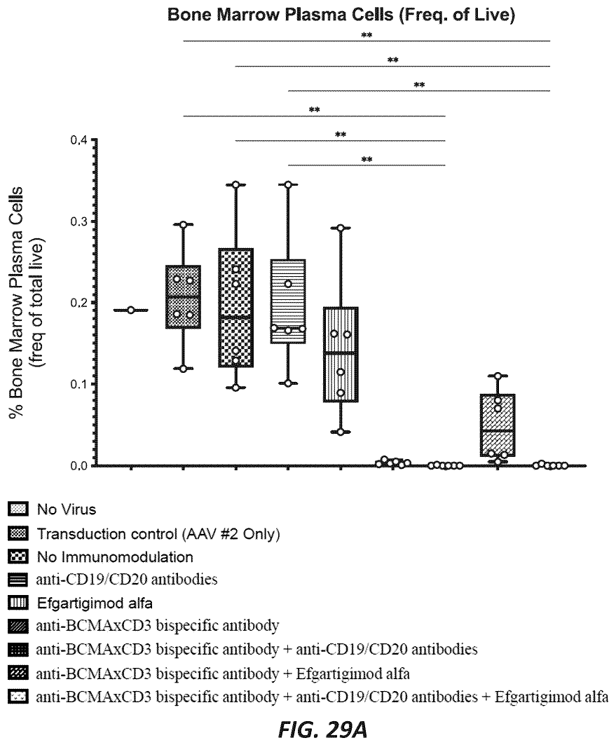

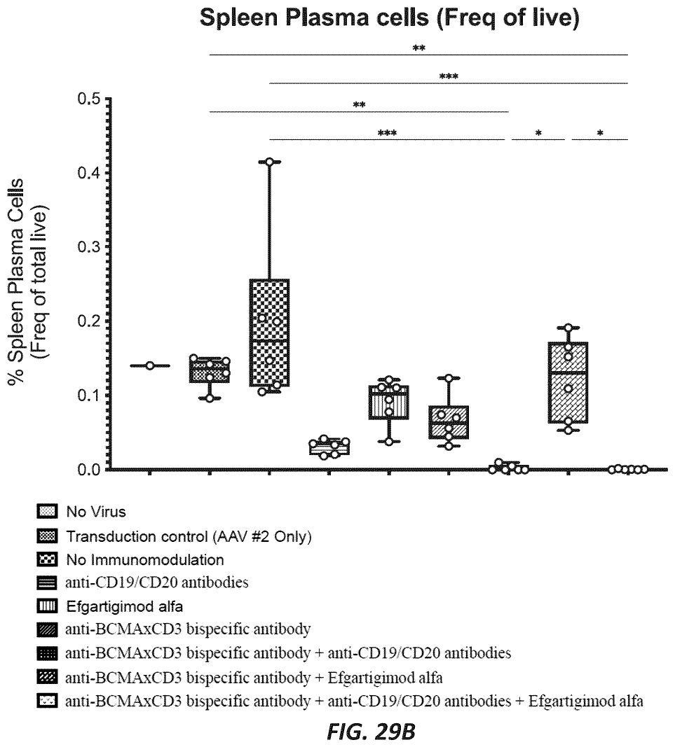

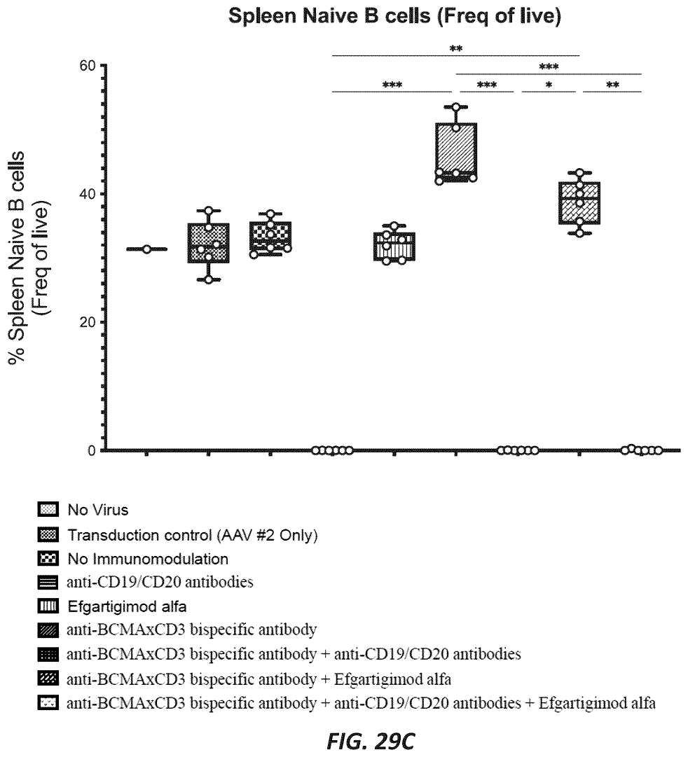

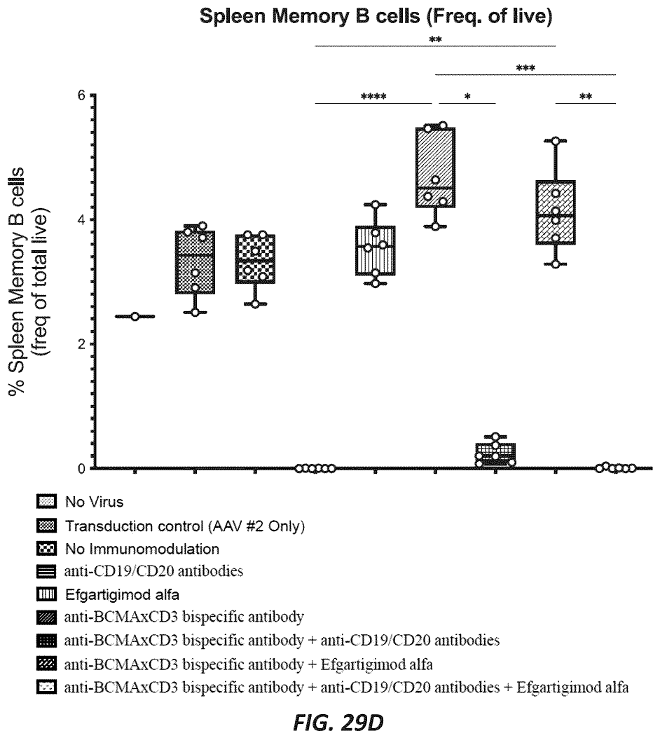

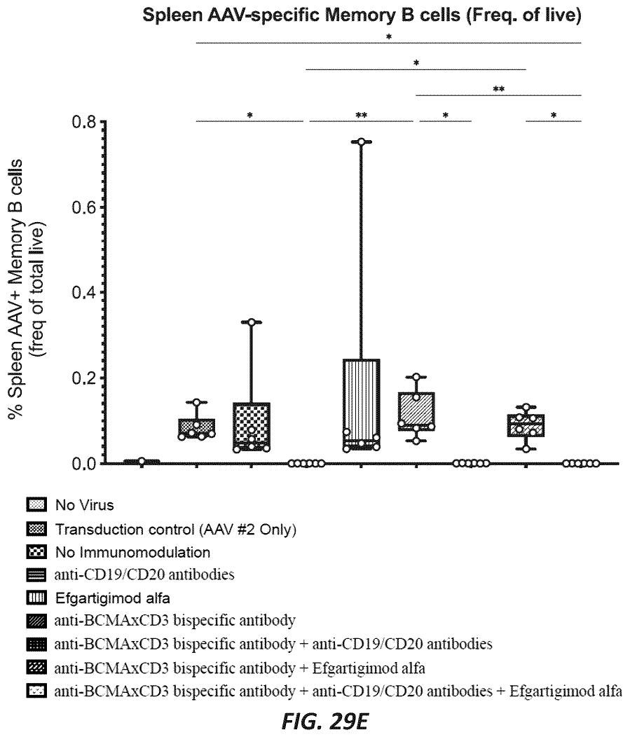

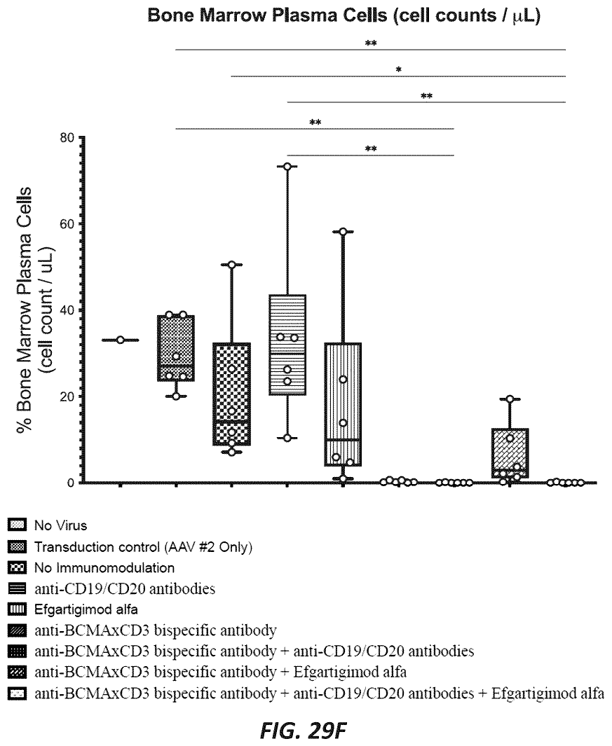

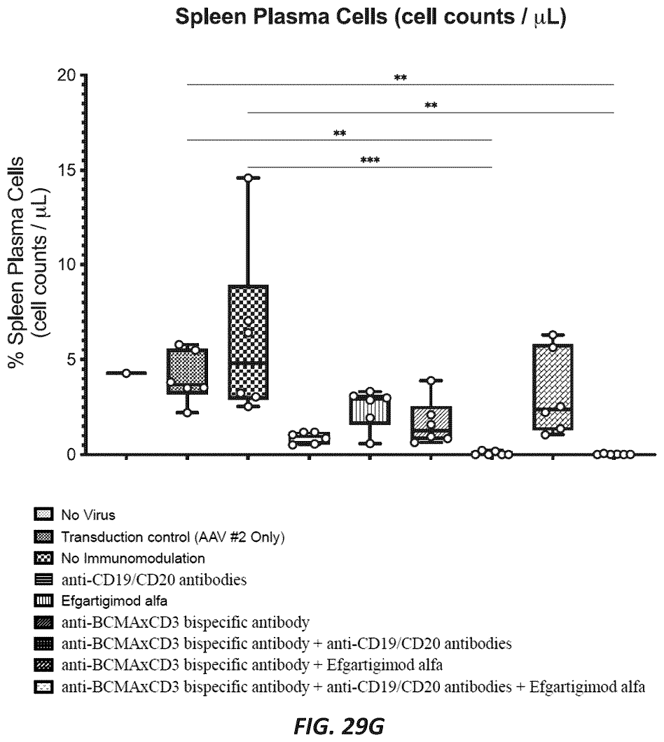

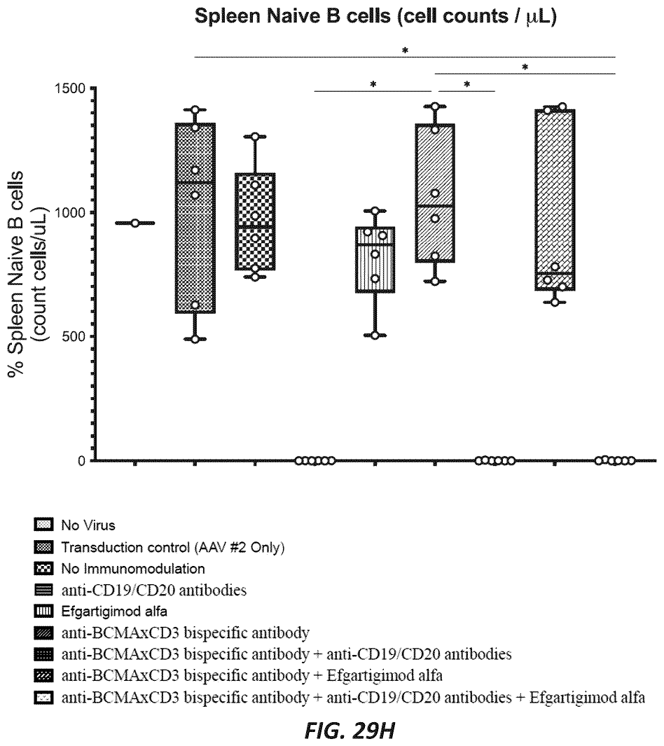

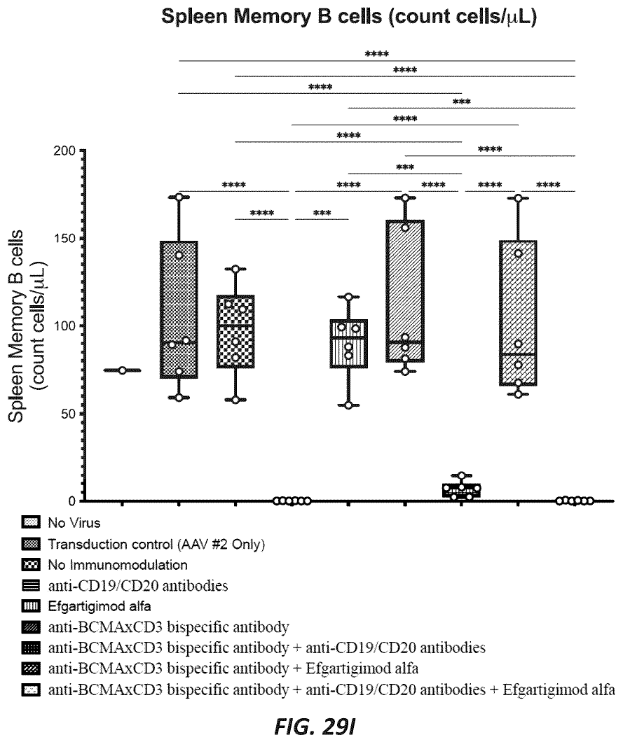

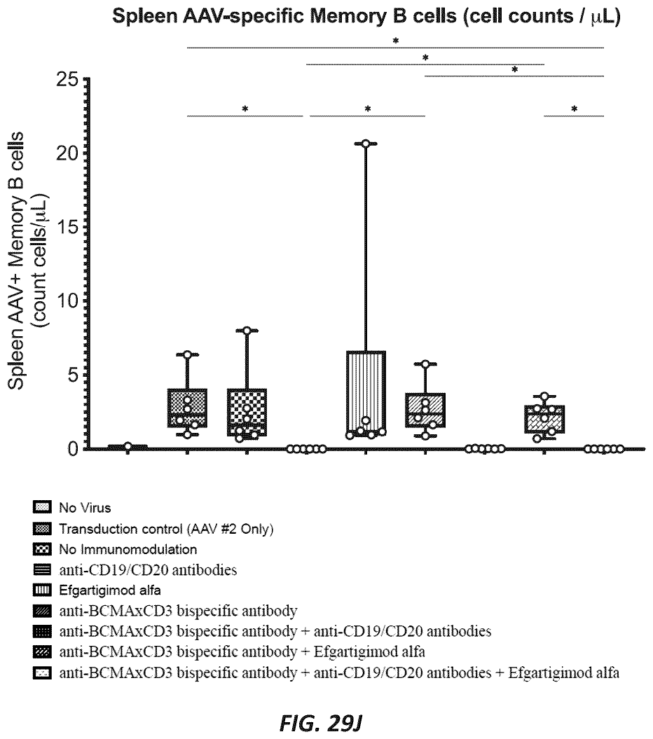

TABLE 47
The flow cytometry antibody staining panel used in FIGS. 29A-29J.
Pre-Stain

| Reagent/Antigen | Conjugate | Reactivity | Host | Clone | Isotype | Supplier |
|---|---|---|---|---|---|---|
| Live/Dead | Blue | N/A | N/A | N/A | N/A | Invitrogen |
| AAV8 | None | N/A | N/A | N/A | N/A | N/A |
| Fc Block (CD16/32) | None | Human | Rat | 2.4G2 | N/A | TONGO |
| | | | | | | biosciences |

Surface stain

| Antigen | Conjugate | Reactivity | Host | Clone | Isotype | Supplier |
|---|---|---|---|---|---|---|
| CD38 | BUV395 | Mouse/Human | Rat | 90/CD38 | IgG2a, k | BD |
| CD138 | BV711 | Mouse | Rat | 281-2 | IgG2a, κ | BD |
| CD95 | BV421 | Mouse | Hamster | Jo2 | IgG2, λ | BD |
| GL-7 | PerCP- | Mouse/Human | Rat | GL7 | IgM, κ | Biolegend |
| | Cy5.5 | | | | | |
| IgD | BV786 | Mouse | Rat | 11-26c.2a | IgG2a, k | BD |
| IgA | FITC | Mouse | Rat | C10-3 | IgG1, κ | BD |
| IgG1 | BV510 | Mouse | Rat | A85-1 | IgG1, κ | BD |
| CD19 | BUV737 | Mouse | Rat | 1D3 | IgG2a, κ | BD |
| B220 | PE-Cy7 | Mouse | Rat | RA3-6B2 | IgG2a, κ | BD |
| CD98 | BV605 | Mouse | Hamster | H202-141 | IgG2a, κ | BD |
| CD1d | BUV563 | Mouse | Rat | WTH2 | IgG2a, κ | BD |
| Biotinylated anti-AAV8 | N/A | | Mouse | ADK8 | IgG2a | Progen |
| IgG | | | | | | |
| TCRβ | APC | Mouse | Hamster | H57-597 | IgG2, λ1 | BD |
| CD200R3 | APC | Mouse | Rat | Ba13 | IgG2a, κ | Biolegend |
| Ly6G | APC | Mouse | Rat | 1A8-Ly6g | IgG2a, κ | eBioscience |
| CD49b | APC | Mouse | Rat | DX5 | IgM, κ | Biolegend |
| CD11b | APC | Mouse | Rat | M1/70 | IgG2b, κ | Biolegend |

| | | | | | | |
|---|---|---|---|---|---|---|
| Reagent | Conjugate | Reactivity | Host | Clone | Isotype | Supplier |

Secondary Stain

| | | | | | | |
|---|---|---|---|---|---|---|
| Streptavidin | PE | N/A | N/A | N/A | N/A | Biolegend |

Intracellular stain

| | | | | | | |
|---|---|---|---|---|---|---|
| Light Chain k | BV650 | Mouse | Rat | 187.1 | IgG1, k | BD |
| Light Chain I | BV650 | Mouse | Rat | R26-46 | IgG2a, κ | BD |
| IgG1 | BV510 | Mouse | Rat | A85-1 | IgG1, k | BD |
| IgA | FITC | Mouse | Rat | C10-3 | IgG1, k | BD |

Analysis of B cell and plasma cell frequencies (FIGS. 29A-29E) and cell counts (FIGS. 29F-29J) in the bone marrow and spleen revealed that plasma cells were fully depleted in groups receiving anti-BCMA×CD3 bispecific antibody+anti-CD19/CD20 antibodies and the triple combination of anti-BCMA×CD3 bispecific antibody+efgartigimod alfa+anti-CD19/CD20 antibodies, consistent with reductions in anti-AAV8 titers seen in these groups. However, plasma cells were incompletely depleted in anti-BCMA×CD3 bispecific antibody groups that did not also receive anti-CD19/CD20 antibodies, suggesting either that ongoing plasma cell formation is a significant contributor to the anti-AAV8 IgG antibody pool in this model, or that mice developed anti-drug antibodies that react with anti-BCMA×CD3 bispecific antibody (a human IgG) that may have limited its therapeutic effect in the absence of B cell depletion (which would also deplete anti-human IgG-specific B cells). The effectiveness of anti-CD19/CD20 antibodies-mediated B cell depletion was also confirmed in analyses of naive, memory, and AAV-specific B cells, with all subsets fully depleted except for a small subset of total memory B cells that were not depleted in the anti-BCMA×CD3 bispecific antibody+anti-CD19/CD20 antibodies combination group. Collectively, these data show that the titer reductions observed in the anti-BCMA×CD3 bispecific antibody+efgartigimod alfa+anti-CD19/CD20 antibodies are consistent with the expected mechanism of action, and that, by comparison, incomplete titer reductions observed in the anti-BCMA×CD3 bispecific antibody+efgartigimod alfa group may be explained by incomplete plasma cell depletion, possibly due to development of anti-drug antibodies reactive with anti-BCMA×CD3 bispecific antibody, efgartigimod, or both.

### Example 31. Negative Impact of Efgartigimod on Anti-BCMA×CD3 Serum Drug Concentration, Which Is Partially Preventable with B Cell Depletion, Is Consistent with Cross-Reactive Anti-Drug Antibody Formation

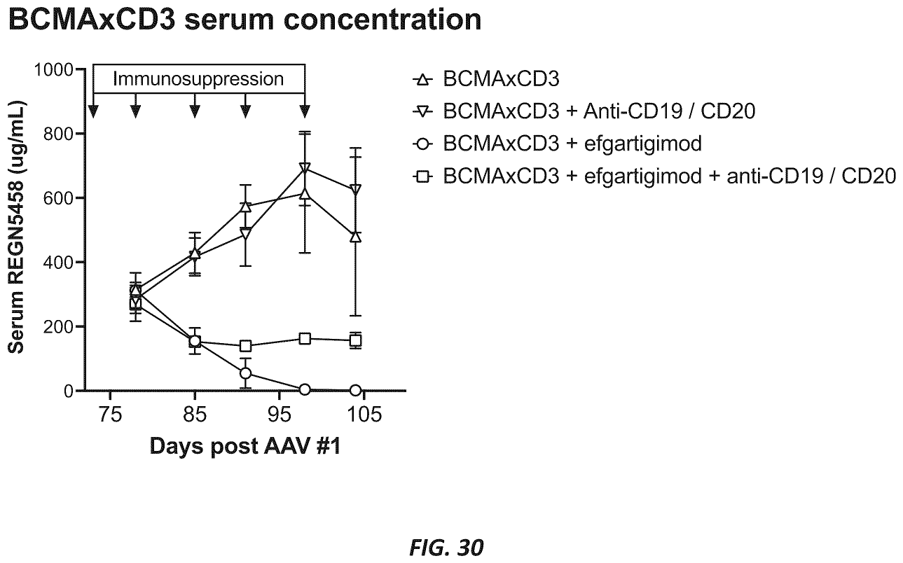

As described in Example 30, robust bone marrow plasma cell depletion was observed in anti-BCMA×CD3, anti-BCMA×CD3+anti-CD19/CD20, and anti-BCMA×CD3+anti-CD19/CD20+efgartigimod treatment groups that was consistent with the expected mechanism of action of anti-BCMA×CD3 observed in previous studies (Limnander et al. (2023) Sci. Transl. Med. 15(726):eadf9561, herein incorporated by reference in its entirety for all purposes). However, mice treated with anti-BCMA×CD3+efgartigimod, but not anti-CD19/CD20, unexpectedly showed incomplete bone marrow plasma cell depletion (FIG. 29A and FIG. 29F). To better understand the impact of efgartigimod on the efficacy of anti-BCMA×CD3, serum anti-BCMA×CD3 concentrations were evaluated during the immunomodulation treatment period. Repeated injections of anti-BCMA×CD3 in anti-BCMA×CD3 and anti-BCMA×CD3+anti-CD19/20 treatment groups resulted in expected increases in serum concentrations of BCMA×CD3 (FIG. 30). Moreover, mice treated with anti-BCMA×CD3+anti-CD19/20+efgartigimod showed significantly lower levels of serum anti-BCMA×CD3, as expected from the mechanism of action of efgartigimod, which blocks FcRn-mediated recycling of IgG, including recycling of therapeutic IgGs such as anti-BCMA×CD3. However, mice receiving anti-BCMA×CD3+efgartigimod without anti-CD19/20 exhibited an even greater rate of anti-BCMA×CD3 antibody clearance that resulted in complete loss of anti-BCMA×CD3 in serum starting 13 days after the initial efgartigimod dose (FIG. 30).

A faster clearance rate, reversible with B cell depletion, suggests that a humoral immune response may be contributing to drug clearance via development of anti-drug antibodies. Efgartigimod is a human IgG1 antibody fragment and is known to be immunogenic in mice. Anti-BCMA×CD3 is a human IgG4, which possesses >90% sequence identity to hIgG1. Therefore, it was concluded that efgartigimod, in the absence of additional B cell depletion, induced human IgG1/IgG4 cross-reactive anti-drug antibodies that accelerated anti-BCMA×CD3 clearance. It was further concluded that this anti-drug antibody response, in the absence of additional B cell depletion, negatively impacted BCMA-mediated bone marrow plasma cell depletion and consequently AAV titer reductions. Thus, the contribution of anti-CD19/20 antibodies to the efficacy of anti-BCMA×CD3 in non-human systems, in the presence of efgartigimod, may be model-specific through prevention of a xenogeneic anti-drug antibody response.

### Example 32. Plasma Cell Depletion in Combination with Neonatal Fe Receptor (FcRn) Blockade Potently Reduces Naturally-Occurring AAV Titers in AAV-Seropositive Cynomolgus Macaques

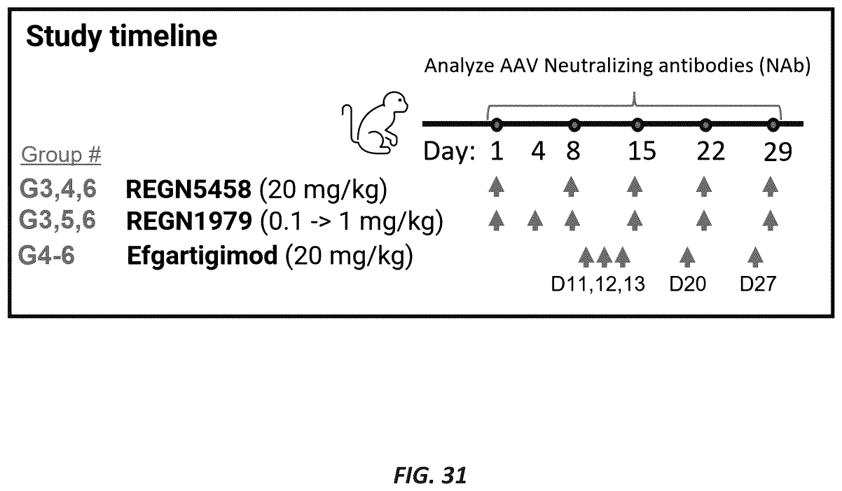

To evaluate whether plasma cell depletion with anti-BCMA×CD3 could similarly suppress naturally-occurring AAV titers arising from exposure to wild-type AAVs, as is common in humans, a non-human primate study was initiated with AAV8-seropositive macaques. Macaques were divided by AAV neutralizing antibody (nAb) titer into five treatment groups (n=3-5 each), each containing animals of high nAb titer (>1:450), as well as one group of seronegative control animals (n=3). Subsequently, animals were treated with various combinations of a plasma cell-depleting bispecific anti-BCMA×CD3 antibody (REGN5458, 20 mg/kg weekly), a B cell-depleting bispecific anti-CD20×CD3 antibody (REGN1979, 0.1 mg/kg on Study Day 1, 1 mg/kg on Study Days 4, 8, and weekly thereafter), and/or an FcRn blocker (efgartigimod, 20 mg/kg on Study Days 11, 12, 13, 20, and 27). Efgartigimod dosing was delayed relative to REGN5458 and REGN1979 dosing to minimize the impact of efgartigimod on REGN5458 and REGN1979 drug half-life due to FcRn blockade and/or cross-reactive anti-drug antibody development. NAb titers were analyzed weekly by cell-based neutralization assay, conducted by VRL Diagnostics (San Antonio, Texas). A schematic of the study design is shown in FIG. 31.

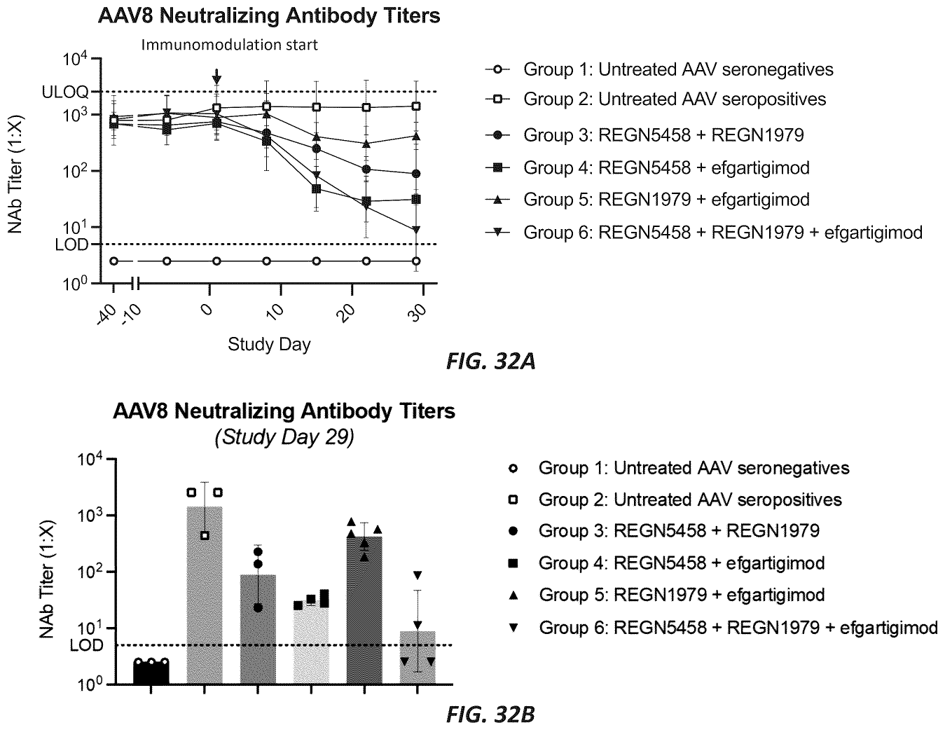

Longitudinal analysis of NAb titers revealed that only groups treated with immunomodulation cocktails containing REGN5458 showed substantive geometric mean titer reductions by ˜4 weeks after the start of immunomodulation. Whereas macaques receiving REGN5458-containing cocktails showed titer reductions of >10-fold, macaques receiving a cocktail containing efgartigimod and REGN1979, but not REGN5458, showed only marginal geometric mean nAb titer reduction of ˜2-fold (FIG. 32A). Similar to findings in mice, cocktails containing both a plasma cell depleter (REGN5458) and an FcRn blocker (efgartigimod), or all three immunomodulators, elicited the greatest titer reductions, with the triple combination of REGN5458, REGN1979, and efgartigimod inducing a >100-fold reduction in nAb titer (FIG. 32A). On Study Day 29, two animals in the triple combination group exhibited nAb titers below the limit of detection of the assay (FIG. 32B), suggesting that these animals could be successfully dosed with an AAV vector. Thus, plasma cell depletion is an effective strategy for suppressing naturally-occurring anti-AAV antibody titers, even of high-titer, to a level that is compatible with AAV dosing.

### Example 33. Titratable Increase in Transgene Expression by Repeated Gene Insertion of a Transgene into a Single Genomic Locus in the Presence of Anti-CD40 Immunomodulation of Anti-AAV Immune Response

Anti-CD40 antibodies or other CD40 inhibitors can be used to prevent or mitigate an anti-AAV antibody response and allow for repeated, titratable dosing of an AAV gene insertion therapeutic. This approach enables a single larger dose to be divided over two or more smaller doses to optimize for therapeutic levels of transgene expression, while also minimizing risk for dose-related toxicities.

In this example, an AAV8 encoding a promoterless, bidirectional DNA template of hFIX and an LNP containing mRNA encoding SpCas9 and a G666 gRNA designed to create a specific double stranded break in mouse albumin intron 1 are dosed two times. The LNP formulation that is used is designed to be minimally immunogenic (e.g., includes an ionizable lipid that does not elicit a strong innate immune response), so as to prevent an adjuvant effect on the immune response to AAV. Prior to and during a time after each AAV+LNP dosing, mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep), which suppresses the AAV antibody response and allows for step-wise, increasing insertion of a hFIX transgene template into the albumin locus of hepatocytes in mice. See, e.g., WO 2023/077012 and US 2023-0149563, each of which is herein incorporated by reference in its entirety for all purposes.

Specifically, mice with native Alb^{+/+} (e.g., CD40-humanized mice) are dosed with anti-CD40 antibody or other CD40 inhibitor before (e.g., 1 week before, on Study Days −7, −3, and −1) and after (e.g., weekly, up to 6 months) the 1^{st} AAV+LNP dosing. The AAV and LNP dose achieves therapeutic levels of gene insertion and hFIX expression, but is low enough that not all hepatocytes or Alb cut sites are modified (e.g., AAV dose between 3.33e11 to 5e13 vg/kg, and LNP dose between 0.3 to 3 mg/kg). After AAV+LNP administration on Study Day 1, splicing of the hFIX coding sequence to albumin exon 1 in hepatocytes results in stable hFIX expression and secretion into the blood, which is detectable by hFIX ELISA conducted two weeks later. When anti-AAV antibody titers are measured in the weeks following AAV+LNP dosing, they are found to be significantly suppressed in mice receiving CD40 blockade (in some cases, completely suppressed) compared to mice dosed in the absence of CD40 blockade, and return to pre-dose (naive) levels by <6 months post treatment (due to CD40 blockade preventing the formation of long-lived memory B cells and plasma cells). One to six months after the initial dose of the AAV+LNP therapeutic (at a time when anti-AAV IgM and anti-AAV IgG titers have returned to below neutralizing levels) the AAV+LNP therapeutic is dosed again at the same dose (or higher or lower, depending on initial levels of gene insertion and desired therapeutic level of expression). Four weeks later, mice are necropsied for analysis of hFIX gene insertion in liver, transgene expression in liver, and hFIX protein levels in blood. Digital PCR analysis of gene insertion at the albumin locus shows that mice treated with CD40 blockade, but not control mice, show a step-wise increase in hFIX transgene insertion (approximately two-fold for mice receiving the same AAV+LNP dose as the first dose). Similarly, RT-qPCR analysis of transgene expression, and ELISA of plasma hFIX, shows approximately double the levels of RNA and protein expression in mice receiving CD40 blockade vs. control mice that did not receive CD40 blockade or mice receiving only a single AAV+LNP dose. Additional dosings of AAV+LNP for increased gene insertion are possible as long as anti-CD40 antibody or other CD40 inhibitor is dosed prior to re-exposure to AAV+LNP therapeutic, and as long there are hepatocytes containing a cut site for the gRNA for the hFIX transgene to be inserted. Thus, this shows that by suppressing both the anti-AAV antibody response and the formation of long-lived immunological memory, CD40 blockade can enable repeat AAV gene insertion of the same transgene at the same genetic locus, allowing for a titratable increase in transgene expression until the desired therapeutic dose is achieved.

### Example 34. Titratable Increase in Transgene Expression by Gene Insertion of the Same Transgene into Distinct Genomic Loci Using Anti-CD40 Antibody Immunomodulation

Using anti-CD40 antibodies or other CD40 inhibitors to mitigate an anti-AAV antibody response can allow for repeated gene insertion of an AAV template into two distinct genomic locations, thus allowing for titratable dosing of an AAV gene insertion therapeutic until the desired therapeutic level of expression is reached.

In this example, an AAV8 template and LNP are co-dosed in two separate instances to achieve repeated gene insertion. In the first instance, the AAV8 template encodes a promoterless, bidirectional DNA template of hFIX, and the LNP contains mRNA encoding SpCas9 and a G666 gRNA designed to create a specific double stranded break in mouse albumin intron 1. In the second instance, the AAV8 template is the same, but the LNP encodes for SpCas9 and a distinct gRNA that targets a separate site, such as another safe harbor site. In both cases, the LNP formulation that is used is designed to be minimally immunogenic (e.g., includes an ionizable lipid that does not elicit a strong innate immune response), so as to prevent an adjuvant effect on the immune response to AAV. Prior to and during the time after each AAV+LNP dosing, mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep), which suppresses the AAV antibody response and allows for step-wise, increasing insertion of a hFIX transgene template into the different target loci in hepatocytes in mice. See, e.g., WO 2023/077012 and US 2023-0149563, each of which is herein incorporated by reference in its entirety for all purposes.

Specifically, mice with native Alb^{+/+} (e.g., CD40 humanized mice) are dosed with anti-CD40 antibody or other CD40 inhibitor before (e.g., 1 week before, on Study Days −7, −3, and −1) and after (e.g., weekly, up to 6 months) the 1^{st} AAV+LNP dosing. The AAV and LNP doses achieve therapeutic levels of gene insertion and hFIX expression (e.g., AAV dose between 3.33e11 to 5e13 vg/kg, and LNP dose between 0.3 to 3 mg/kg). After AAV+LNP administration on Study Day 1, splicing of the hFIX coding sequence to albumin exon 1 in hepatocytes leads to stable hFIX expression and secretion into the blood, which is detectable by hFIX ELISA conducted two weeks later. When anti-AAV antibody titers are measured in the weeks following AAV+LNP dosing, they are found to be significantly suppressed in mice receiving CD40 blockade (in some cases, completely suppressed) compared to mice dosed in the absence of CD40 blockade, and return to pre-dose (naive) levels by <6 months post treatment (due to CD40 blockade preventing the formation of long-lived memory B cells and plasma cells). One to six months after initial dose of the AAV+LNP therapeutic (at a time when anti-AAV IgM and anti-AAV IgG titers have returned to below neutralizing levels) the second AAV+LNP therapeutic is dosed at the same dose (or higher or lower, depending on initial levels of gene insertion and desired therapeutic level of expression). This creates another double strand break and facilitates a second gene insertion event at a distinct genomic location in hepatocytes (e.g., other safe harbor site). Expression of the second (identical) hFIX transgene occurs via splicing of the hFIX coding sequence to the exon containing the protein secretion signal. As positive controls for gene insertion, separate groups of mice receive either the first AAV+LNP vector or the second AAV+LNP vector only. Four weeks later, mice are necropsied for analysis of hFIX gene insertion in liver, transgene expression in liver, and hFIX protein levels in blood. Digital PCR analysis of gene insertion shows successful insertion of hFIX at both the albumin locus and the second insertion site (e.g., other safe harbor locus) in mice treated with CD40 blockade, but not control mice. RT-qPCR analysis of transgene expression, and ELISA of plasma hFIX, shows approximately double the levels of RNA and protein expression in CD40 blockade-treated mice versus control mice that did not receive CD40 blockade, or versus mice receiving only a single AAV+LNP dose. Additional dosings of AAV+LNP for increased gene insertion are possible at other genomic sites as long as anti-CD40 antibody or other CD40 inhibitor is dosed prior to re-exposure to AAV+LNP therapeutic. Thus, this shows that by suppressing both the anti-AAV antibody response and the formation of long-lived immunological memory, CD40 blockade can enable repeat AAV gene insertion of the same transgene at distinct genomic loci, allowing for a titratable increase in transgene expression until the desired therapeutic dose is achieved.

### Example 35. Sequential Gene Insertion for Expression of Two Different Protein Products Using Anti-CD40 Antibody Immunomodulation

Using anti-CD40 antibodies or other CD40 inhibitors to mitigate an anti-AAV antibody response can allow for sequential gene insertion of two distinct AAV templates, encoding two distinct transgene protein proteins, into distinct genomic locations.

In this example, an AAV8 template and LNP are co-dosed in two separate instances to achieve repeated gene insertion at two distinct genomic locations. In the first instance, the AAV8 template encodes a promoterless, bidirectional DNA template of hFIX, and the LNP contains mRNA encoding SpCas9 and a G666 gRNA specific for creating a specific double stranded break in mouse albumin intron 1. In the second instance, the AAV8 template encodes a promoterless DNA template encoding a human IgG1 monoclonal antibody, and the LNP contains mRNA encoding SpCas9 and a distinct gRNA that targets a separate site (e.g., another safe harbor site). In both cases, the LNP formulation that is used is designed to be minimally immunogenic (e.g., includes an ionizable lipid that does not elicit a strong innate immune response), so as to prevent an adjuvant effect on the immune response to AAV. Prior to and during the time after each AAV+LNP dosing, mice are treated with anti-CD40 antibody (e.g., REGN16335, iscalimab, ravagalimab, BI-655064, or abiprubart) or other CD40 inhibitor (e.g., CD40L binding protein such as tegoprubart of dazodalibep), which suppresses the AAV antibody response.

Specifically, mice with native Alb^{+/+} (e.g., CD40 humanized mice) are dosed with anti-CD40 antibody or other CD40 inhibitor before (e.g., 1 week before, on Study Days −7, −3, and −1) and after (e.g., weekly, up to 6 months) the 1^{st} AAV+LNP dosing. The AAV and LNP doses achieve therapeutic levels of gene insertion and hFIX expression (e.g., AAV dose between 3.33e11 to 5e13 vg/kg, and LNP dose between 0.3 to 3 mg/kg). After AAV+LNP administration on Study Day 1, splicing of the hFIX coding sequence to albumin exon 1 in hepatocytes leads to stable hFIX expression and secretion into the blood, which is detectable by hFIX ELISA two weeks later. When anti-AAV antibody titers are measured in the weeks following AAV+LNP dosing, they are found to be significantly suppressed in mice receiving CD40 blockade (in some cases, completely suppressed) compared to mice dosed in the absence of CD40 blockade, and return to pre-dose (naive) levels by <6 months post treatment (due to CD40 blockade preventing the formation of long-lived memory B cells and plasma cells). One to six months after initial dose of the AAV+LNP therapeutic (at a time when anti-AAV IgM and anti-AAV IgG titers have returned to below neutralizing levels) the second AAV+LNP therapeutic is dosed at the same dose (or alternative dose appropriate for desired expression level of the second transgene). This creates another double strand break and facilitates the second gene insertion event of the therapeutic human IgG1 antibody. Expression of human IgG1 transgene occurs via splicing of the coding sequence to the exon containing the protein secretion signal. As positive controls for gene insertion, separate groups of mice receive either the first AAV+LNP combination or the second AAV+LNP combination only. Four weeks later, mice are necropsied for analysis of hFIX and human IgG1 gene insertion in liver, transgene RNA expression in liver, and protein levels in blood. Digital PCR analysis of gene insertion at the first locus (albumin) and second locus (e.g., other safe harbor site) shows that mice treated with CD40 blockade, but not control mice, exhibit successful insertion of the two transgenes at their respective loci (e.g., hFIX at albumin, and human IgG1 at another safe harbor site). Similarly, RT-qPCR analysis of hFIX and human IgG1 transgene expression, and hFIX and human IgG1 ELISA of plasma, shows that mice that receive CD40 blockade exhibit co-expression of both transgenes. By contrast, mice that receive both AAV+LNP dosings, but do not receive CD40 blockade, show expression of only the first transgene (hFIX). Alternatively, control mice that receive either one of the AAV+LNP dosing (but not both) exhibit expression of either transgene alone. Additional dosings of AAV+LNP to achieve gene insertion of additional transgenes at other genomic sites are possible as long as anti-CD40 antibody or other CD40 inhibitor is dosed prior to re-exposure to AAV+LNP therapeutic. Thus, this shows that by suppressing both the anti-AAV antibody response and the formation of long-lived immunological memory, CD40 blockade can enable repeat AAV gene insertion of distinct transgenes at distinct genomic loci.

### Example 36. Prophylactic CD40 Blockade with Anti-CD40 mAb Suppresses the IgM, IgG, and Neutralizing Antibody (NAb) Response to AAV in Non-Human Primates

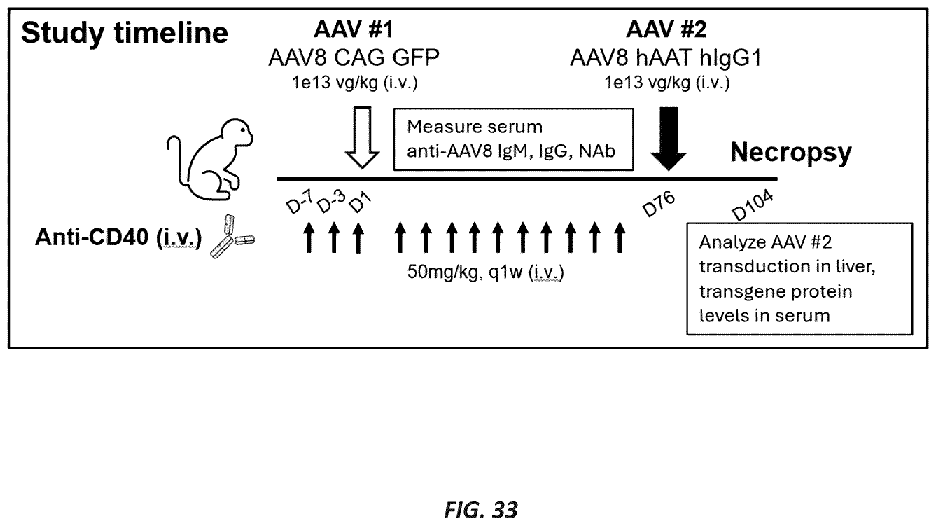

As demonstrated in Examples 12-13, prophylactic CD40 blockade with anti-CD40 mAb potently suppresses anti-AAV antibody responses and enables systemic AAV vector re-administration in mice. To evaluate the ability of CD40 blockade to suppress antibody responses to AAV vectors in larger mammals, a similar study was conducted in cynomolgus macaques. Specifically, AAV8-seronegative cynomolgus macaques were divided into groups of 4-6 animals receiving either no immunomodulation (Groups 1 and 2) or the anti-CD40 antagonist mAb (Group 3; COMP11209, having the CDRs set forth in SEQ ID NOS: 135-140, 50 mg/kg i.v.). After three initial loading doses of anti-CD40 mAb on Study Days −7, −3, and 1, animals in Groups 2 and 3 were dosed intravenously on Study Day 1 with an AAV8 vector (“AAV #1”) encoding eGFP at 1e13 vector genomes per kilogram (vg/kg). Animals in Group 3 continued to receive anti-CD40 mAb weekly at the same dose until Study Day 76. Blood was sampled periodically for longitudinal antibody titer analysis. A schematic of the study design is shown in FIG. 33.

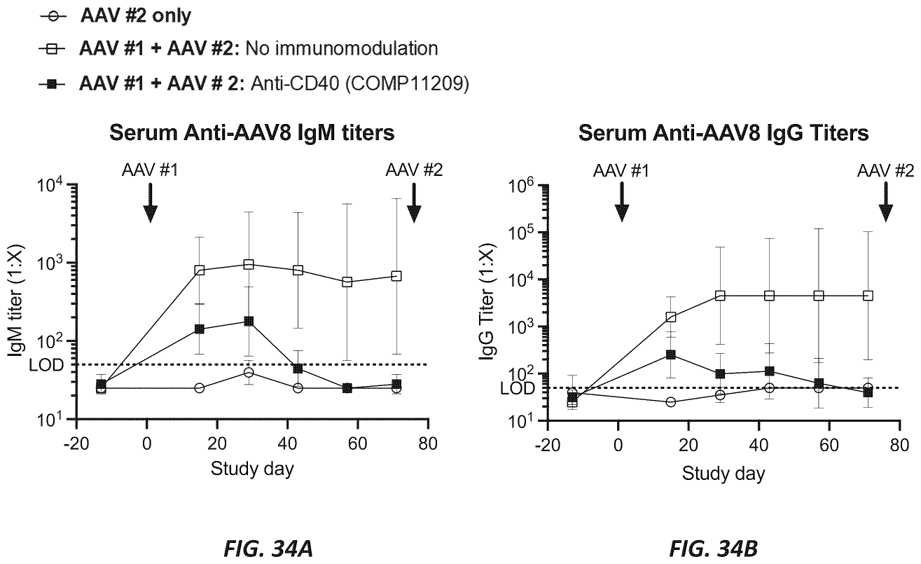

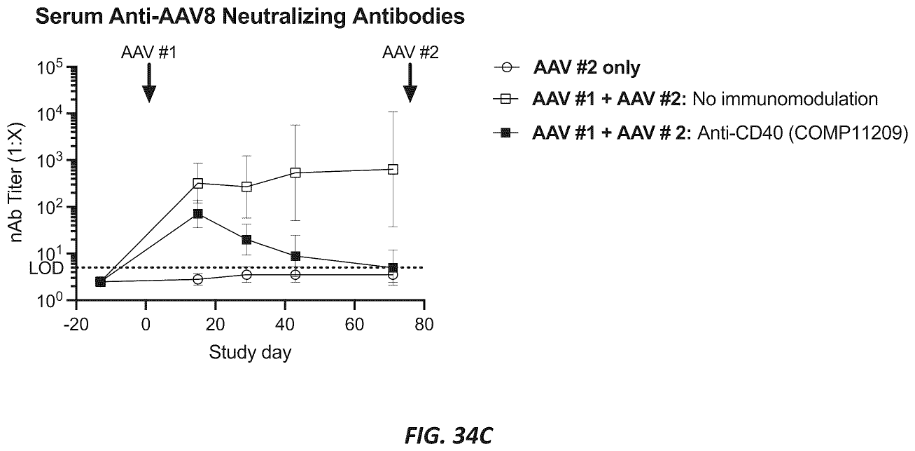

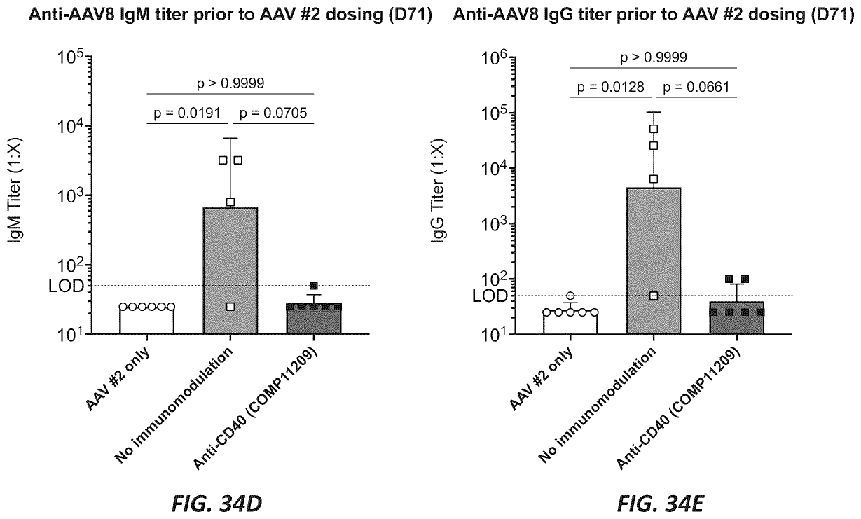

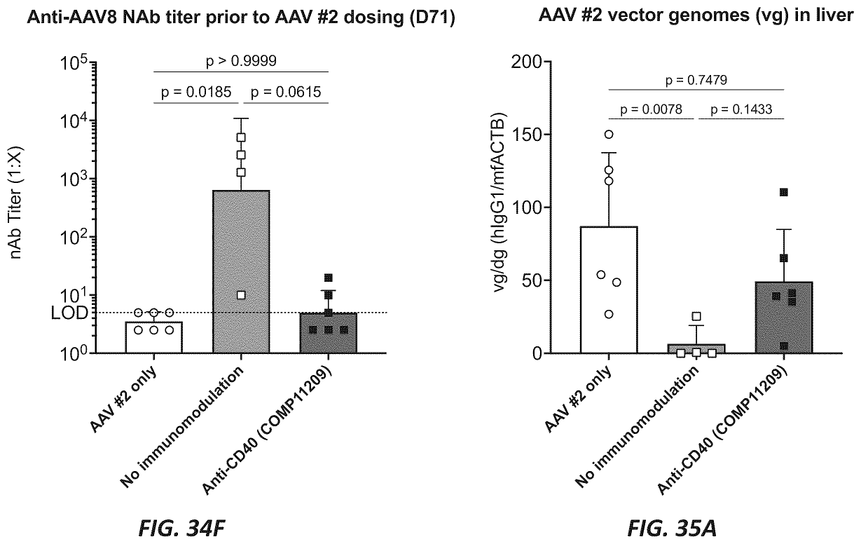

Analysis of serum anti-AAV IgM, IgG, and neutralizing antibody (NAb) titers was conducted by anti-AAV IgM/IgG titer ELISA and cell-based neutralization assay, according to standard methods utilized by the University of Pennsylvania Gene Therapy Program Immunology Core. Results indicated that, as expected, macaques receiving no antibody (Group 2, “No immunomodulation”) mounted robust anti-AAV8 IgM, IgG, and NAb responses in the weeks following AAV exposure that were sustained in most animals for the entire ˜10 week sampling period (FIGS. 34A-34C). By contrast, anti-CD40 mAb-treated animals showed significantly reduced antibody responses, exhibiting only transient IgM, IgG, and NAb responses that were lower in magnitude and waned to undetectable or near-undetectable levels over the course of the ˜10 week sampling period. By Study Day 71 anti-CD40 mAb-treated animals, but not control animals, recorded geometric mean titers below the limit of detection (FIGS. 34D-34F). Thus, these data show that antibody-mediated CD40 blockade is an effective strategy to suppress the magnitude and duration of anti-AAV8 antibody responses following AAV vector treatment in non-human primates.

### Example 37. Prophylactic CD40 Blockade with Anti-CD40 mAb Enables Systemic AAV Re-Dosing in Non-Human Primates

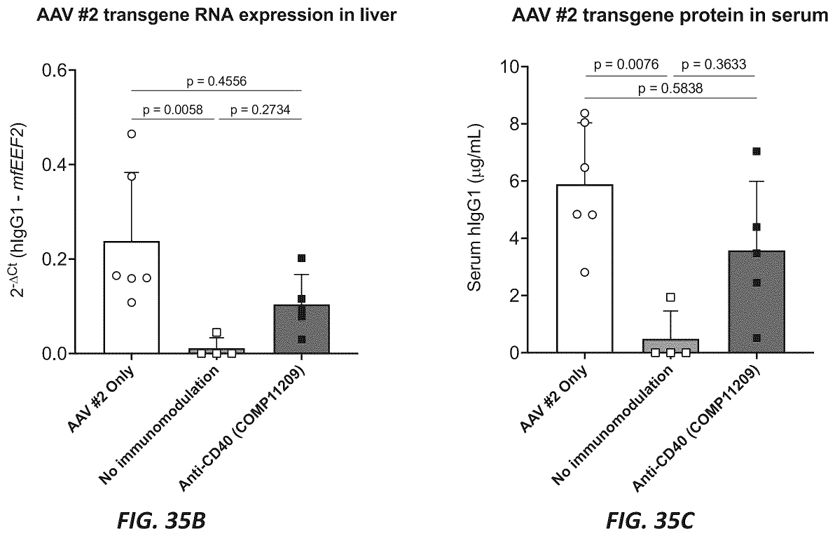

In this example, the ability of anti-CD40 mAb-treatment to enable systemic AAV vector re-administration was evaluated. All animals from Example 36, including those that had not previously received AAV #1 (Group 1), were intravenously administered a second AAV8 vector, (“AAV #2”) encoding a secreted human IgG1 monoclonal antibody transgene from a liver-specific promoter, at 1e13 vg/kg on Study Day 76. Four weeks later (Study Day 104), animals were necropsied, and liver transduction was evaluated by digital PCR. As expected, few vector genomes were detected in control animals that previously received AAV #1 but no immunomodulation (FIG. 35A). By contrast, anti-CD40 mAb-treated animals showed evidence of strong transduction with the second AAV vector, exhibiting vector genome levels approaching that of Group 1 transduction control animals that received AAV #2 only. Analysis of transgene RNA (by Tagman RT-qPCR) and serum transgene protein (by hIgG1 ELISA) showed similar findings, with anti-CD40 mAb-treated animals, but not re-dosed animals receiving no antibody, exhibiting readily detectable levels of transgene RNA and serum hIgG1 protein (FIGS. 35B-35C). Thus, these findings show that systemic AAV vector re-administration is achievable via prophylactic antibody-mediated CD40 blockade in non-human primates.

### Example 38. Prophylactic CD40 Blockade with Anti-CD40 mAb Reduces Liver Injury Associated with Anti-Transgene T Cell Responses, and Results in Increased Transgene Expression in Non-Human Primates

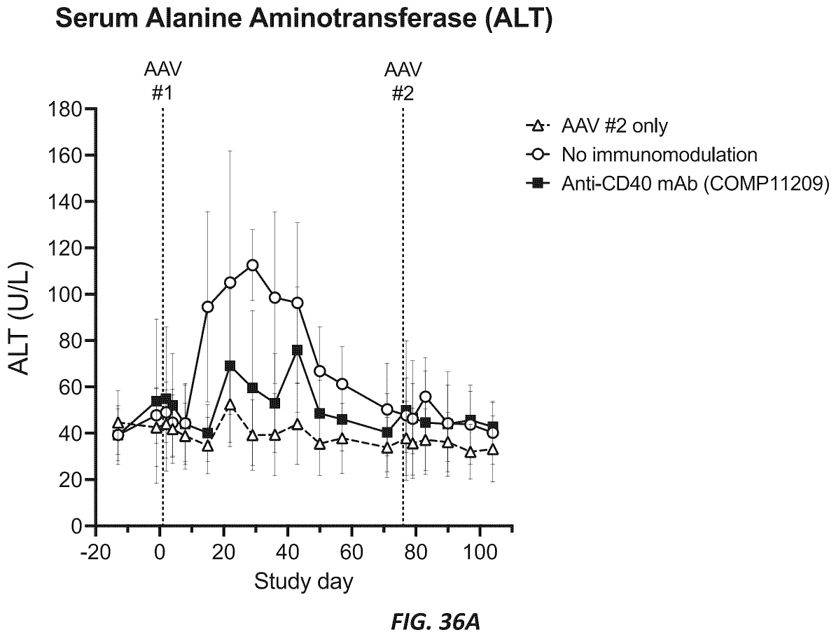

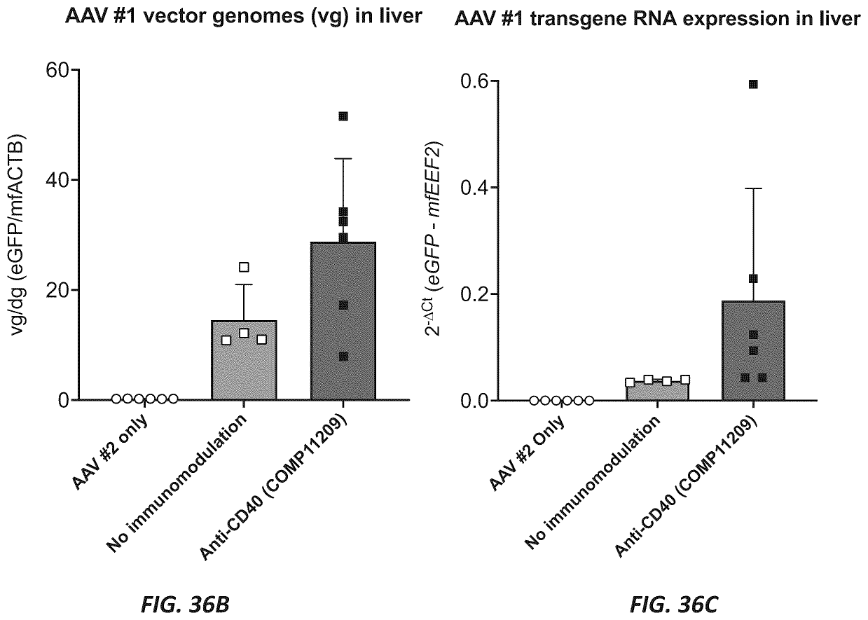

T cell responses to transgene and AAV capsid have previously been shown to be associated with liver injury and loss of transgene expression in liver-directed AAV gene therapies (Gao et al. (2009) Hum. Gene Ther. 20(9):930-942 and Manno et al. (2006) Nat. Med. 12(3):342-347). To evaluate whether CD40 blockade can mitigate T cell-mediated liver injury post AAV treatment, cynomolgus macaques from Examples 36-37 were longitudinally evaluated for alanine aminotransferase activity (ALT) in serum, a common clinical measurement of liver injury. As expected, AAV #1, which encoded the immunogenic transgene eGFP, resulted in transient ALT elevations in control animals treated with AAV but not antibody (FIG. 36A), as has previously been observed (Gao et al. (2009) Hum. Gene Ther. 20(9):930-942). In contrast, anti-CD40 mAb-treated animals showed lower average ALT levels following AAV #1 dosing. Moreover, consistent with a reduced T cell response, at the time of necropsy on Study Day 104, anti-CD40 mAb-treated animals exhibited higher mean levels of AAV #1 vector genomes and eGFP RNA compared to control animals that did not receive anti-CD40 mAb (FIGS. 36B-36C). Thus, these data suggest that anti-CD40 mAb blockade is an effective strategy to attenuate liver injury and transgene loss associated with anti-transgene T cell immune responses post AAV dosing.

### Example 39. Use of Anti-CD40 Antagonist Antibodies to Prevent an Immune Response to IgG Degrading Enzyme (IdeS) and Therefore Enable Repeated IdeS Administration and AAV Gene Insertion

The IgG degrading enzyme IdeS has previously been shown to transiently reduce anti-AAV IgG through IgG cleavage (Leborgne et al. (2020) Nat. Med. 26(7):1096-1101). Treatment of animals with IdeS in vivo has emerged as a promising strategy to transiently deplete anti-AAV IgGs in circulation, thereby in some cases enabling AAV transduction, and in some cases, re-transduction. However, because IdeS is of bacterial origin and therefore immunogenic, the ability to re-dose IdeS to enable repeated rounds of AAV gene insertion in seropositive subjects remains unclear.

In this example, anti-CD40 antagonist antibodies are administered to AAV8-seropositive cynomolgus macaques prior to IdeS administration to prevent an antibody response to IdeS. The CD40 antibodies are resistant to IdeS cleavage (e.g. through use of an engineered antibody isotype that carries mutations within the known cleavage site of IdeS (e.g. within the C_{H}2 hinge sequence (CPAPELLGGPSVF (SEQ ID NO: 342)) or by swapping this sequence with the homologous sequence in another IdeS-resistant species such as mouse) and therefore maintain efficacy in the presence of IdeS. The administration of IdeS results in cleavage of circulating IgGs, including anti-AAV8 IgGs. Analysis of anti-AAV8 IgGs and neutralizing antibodies 2-4 days later confirms that IdeS effectively reduced anti-AAV8 IgGs to levels that are below neutralizing levels. Subsequently, an AAV8 gene insertion template vector (e.g., encoding a promoterless human FIX transgene)+LNP containing mRNA encoding SpCas9+ relevant gRNA (e.g., targeting a safe harbor site, such as albumin intron 1) is subsequently dosed, enabling gene insertion. Successful gene insertion and expression is evaluated at some time post dosing (e.g., for human FIX, 1-2 weeks later in blood by ELISA). Analysis of anti-IdeS titers 2-4 weeks after IdeS dosing confirms suppression of antibody response to IdeS. At some time after the first AAV (e.g., 2 months later), at a time when the animals are confirmed to be AAV seropositive again (due to the transient nature of IdeS activity), anti-CD40 antibodies are again administered, followed by IdeS. Once again, 2-4 days later, analysis of anti-AAV8 IgGs and neutralizing antibodies confirms that IdeS effectively reduces anti-AAV8 IgGs to below neutralizing levels. A second AAV8 gene insertion template vector (e.g., encoding a promoterless therapeutic human IgG1)+LNP containing mRNA encoding SpCas9+ relevant gRNA (e.g., targeting a second safe harbor site) is subsequently dosed, enabling gene insertion for a second time. Successful gene insertion and expression is evaluated at some time post dosing (e.g., for human IgG1 transgene, 1-4 weeks later in blood by ELISA). Analysis of anti-IdeS titers 2-4 weeks after the second IdeS dosing confirms suppression of antibody response to IdeS. Additional AAVs can be re-administered, as long as IdeS is administered prior to dosing, and as long as anti-CD40 antibodies are administered prior to IdeS administration to prevent an antibody response to IdeS.

1. A method of inserting a nucleic acid encoding a polypeptide of interest into a target genomic locus in a cell or a population of cells in a subject, comprising administering to the subject:
(a) a nucleic acid construct comprising a coding sequence for the polypeptide of interest;
(b) a nuclease agent or one or more nucleic acids encoding the nuclease agent, wherein the nuclease agent targets a nuclease target site in the target genomic locus; and
(c) a CD40 inhibitor,
wherein the nuclease agent cleaves the nuclease target site, and the nucleic acid construct is inserted into the target genomic locus.

2. A method of expressing a polypeptide of interest from a target genomic locus in a cell or a population of cells in a subject, comprising administering to the subject:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
(c) a CD40 inhibitor,
wherein the nuclease agent cleaves the nuclease target site, the nucleic acid construct is inserted into the target genomic locus to create a modified target genomic locus, and the polypeptide of interest is expressed from the modified target genomic locus.

3. A method of treating an enzyme deficiency in a subject in need thereof, comprising administering to the subject:
(a) a nucleic acid construct comprising a coding sequence for a polypeptide of interest, wherein the polypeptide of interest comprises an enzyme to treat the enzyme deficiency;
(b) a nuclease agent or one or more nucleic acids encoding the nuclease agent, wherein the nuclease agent targets a nuclease target site in a target genomic locus; and
(c) a CD40 inhibitor,
wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

4. A method of preventing or reducing the onset of a sign or symptom of an enzyme deficiency in a subject in need thereof, comprising administering to the subject:
(a) a nucleic acid construct comprising a coding sequence for a polypeptide of interest, wherein the enzyme deficiency is characterized by a loss-of-function of the polypeptide of interest;
(b) a nuclease agent or one or more nucleic acids encoding the nuclease agent, wherein the nuclease agent targets a nuclease target site in a target genomic locus; and
(c) a CD40 inhibitor,
wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

5.-156. (canceled)

157. A composition or combination comprising a CD40 inhibitor in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

158.-269. (canceled)

270. A kit comprising the composition or combination of claim 157.

271. (canceled)
